(19) United States
(12) Patent Application Publication
(10) Pub. No.: US 2025/0019377 A1
(43) Pub. Date: Jan. 16, 2025

(54) 1, 4-OXAZEPANE DERIVATIVES AND USES THEREOF

(71) Applicant: JACOBIO PHARMACEUTICALS CO., LTD, Beijing (CN)

(72) Inventors: Hongwei YANG, Beijing (CN); Cunbo MA, Beijing (CN); Panliang GAO, Beijing (CN); Huifeng HAN, Beijing (CN); Peng WANG, Beijing (CN); Runze LI, Beijing (CN); Xiaoyu LIU, Beijing (CN); Yanping WANG, Beijing (CN); Wei LONG, Beijing (CN); Wei ZHANG, Beijing (CN)

(21) Appl. No.: 18/684,205

(22) PCT Filed: Aug. 17, 2022
(86) PCT No.: PCT/CN2022/112919

§ 371 Date: Feb. 16, 2024

(30) Foreign Application Priority Data
Aug. 18, 2021 (WO) PCT/CN2021/113365
Oct. 12, 2021 (WO) PCT/CN2021/123382
Oct. 13, 2021 (WO) PCT/CN2021/123604
Oct. 14, 2021 (WO) PCT/CN2021/123884
Nov. 22, 2021 (WO) PCT/CN2021/132070
Dec. 10, 2021 (WO) PCT/CN2021/137092
Feb. 24, 2022 (WO) PCT/CN2022/077678
Mar. 15, 2022 (WO) PCT/CN2022/081022
Mar. 31, 2022 (WO) PCT/CN2022/084273
Mar. 31, 2022 (WO) PCT/CN2022/084321
Apr. 14, 2022 (WO) PCT/CN2022/086755
Apr. 18, 2022 (WO) PCT/CN2022/087382
Apr. 18, 2022 (WO) PCT/CN2022/087383

(51) Int. Cl. C07D 471/04; A61K 31/553; A61P 35/00; C07D 413/14; C07D 487/04; C07D 519/00

(52) U.S. Cl. CPC C07D 471/04; A61K 31/553; A61P 35/00; C07D 413/14; C07D 487/04; C07D 519/00

(57) ABSTRACT

A Compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof is disclosed:

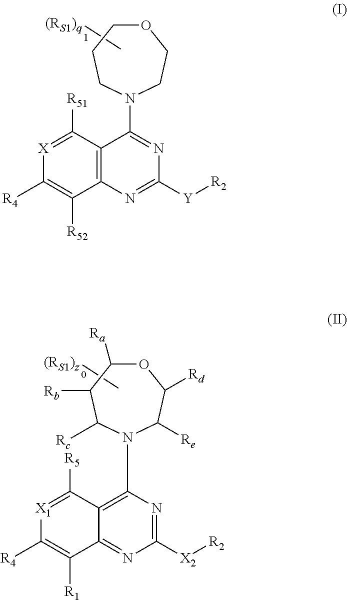

### CROSS-REFERENCES TO RELATED APPLICATIONS

This application claims the benefit of priority to PCT/CN2021/113365, filed on Aug. 18, 2021; PCT/CN2021/123382, filed on Oct. 12, 2021; PCT/CN2021/123604, filed on Oct. 13, 2021; PCT/CN2021/123884, filed on Oct. 14, 2021; PCT/CN2021/132070, filed on Nov. 22, 2021; PCT/CN2022/077678, filed on Feb. 24, 2022; PCT/CN2022/084321, filed on Mar. 31, 2022; PCT/CN2021/137092, filed on Dec. 10, 2021; PCT/CN2022/087382, filed on Apr. 18, 2022; PCT/CN2022/081022, filed on Mar. 15, 2022; PCT/CN2022/084273, filed on Mar. 31, 2022; PCT/CN2022/086755, filed on Apr. 14, 2022; PCT/CN2022/087383, filed on Apr. 18, 2022, and all of which are hereby incorporated herein by reference in their entireties.

### TECHNICAL FIELD

The invention relates to compounds that inhibit the activity of multiple forms of K-Ras protein including K-Ras wild type and K-Ras mutant types, compositions comprising the same, and the methods of using the same.

### BACKGROUND ART

Kirsten Rat Sarcoma 2 Viral Oncogene Homolog (“K-Ras”) is a small GTPase and a member of the RAS family of oncogenes. K-Ras serves as a molecular switch cycling between inactive (GDP-bound) and active (GTP-bound) states to transduce upstream cellular signals received from multiple tyrosine kinases to downstream effectors to regulate a wide variety of processes, including cellular proliferation. Aberrant expression of K-Ras accounts for up to ˜20% of all cancers and oncogenic K-Ras mutations that stabilize GTP binding and lead to constitutive activation of K-Ras. K-Ras mutations at codons 12, 13, 61 and other positions of the K-Ras primary amino acid sequence are present in 88% of all pancreatic adenocarcinoma patients, 50% of all colorectal adenocarcinoma patients, and 32% lung adenocarcinoma patients. A recent publication also suggested wild type K-Ras inhibition could be a viable therapeutic strategy to treat K-Ras wild type dependent cancers.

Allele-specific K-Ras G12C inhibitors, such as sotorasib (AMG510) or adagrasib (MRTX849), are currently changing the treatment paradigm for patients with K-Ras G12C mutated non-small cell lung cancer and colorectal cancer. The success of addressing a previously elusive K-Ras allele has fueled drug discovery efforts for all K-Ras mutants. Multiple K-Ras inhibitors have the potential to address broad patient populations, including K-Ras G12C, K-Ras G12D, K-Ras G12V, K-Ras G13D, K-Ras G12R, K-Ras G12S, K-Ras G12A, K-Ras Q61H mutant and K-Ras wild type amplified cancers.

Therefore, there are unmet needs to develop new multiple K-Ras inhibitors for treating K-Ras mediated cancers.

### SUMMARY OF THE INVENTION

Provided herein is a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof:

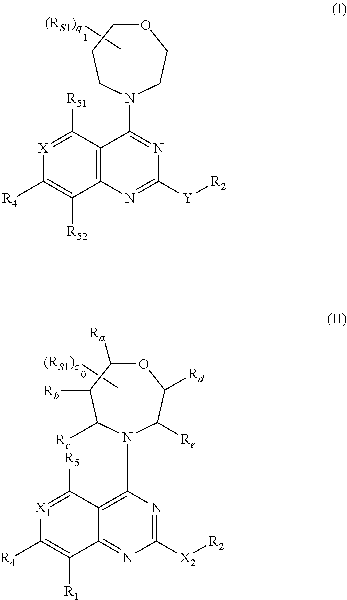

Wherein, the definition of each of variables is as below.

Also provided herein is a pharmaceutical composition comprising a therapeutically effective amount of a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof as defined herein; and a pharmaceutically acceptable excipient.

Also provided herein is a method for treating cancer in a subject comprising administering a therapeutically effective amount of a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof as defined herein to a subject in need thereof.

Also provided herein is a method for treating cancer in a subject in need thereof, the method comprising (a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and (b) if so, administering a therapeutically effective amount of a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof, or a pharmaceutical composition as defined herein to the subject in need thereof.

Also provided herein is a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof, or a pharmaceutical composition as defined herein for use in therapy.

Also provided herein is a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof, or a pharmaceutical composition as defined herein for use as a medicament.

Also provided herein is a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof, or a pharmaceutical composition as defined herein for use in a method for the treatment of cancer.

Also provided herein is a use of a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof, or a pharmaceutical composition as defined herein for the treatment of cancer.

Also provided herein a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof, or a pharmaceutical composition as defined herein for the manufacture of a medicament for the treatment of cancer.

Also provided herein is a process for preparing a compound of formula (I) or (II) as defined herein.

Also provided herein is an intermediate for preparing a compound of formula (I) or (II) as defined herein.

### DETAILED DESCRIPTION

Provided herein are the following disclosures:

[1]. A compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof:

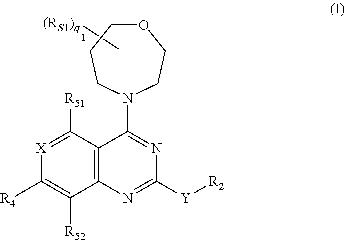

### Wherein,

X is selected from N or CR₅₃;
R_{S1} at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein, said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S1} together with the carbon atom to which they are both attached form

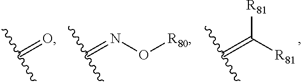

a 3-20 membered carbocyclic ring or a 3-20 heterocyclic ring; wherein, said 3-20 membered carbocylic ring or 3-20 heterocyclic ring is optionally substituted with one or more R₁₆ₐ;
Optionally, two adjacent R_{S1} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R_{16b};
Optionally, two nonadjacent R_{S1} are connected together to form a C₀₋₆ alkylene bridge, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R_{16c};
q₁ is independently selected from 0, 1, 2, 3, 4, 5, 6, 7, 8, 9 or 10;
R₂ is selected from -L₅-(3-12 membered heterocyclyl), -L₅-(3-12 membered cycloalkyl), -L₅-(6-12 member aryl), -L₅-(5-12 membered heteroaryl), -L₅-N(R₇₅)₂,

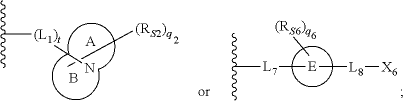

Each of L₅ at each occurrence is independently selected from a bond or C₁₋₁₀ alkylene optionally substituted with one or more R₁₆ₙ;
Said 3-12 membered heterocyclyl in -L₅-(3-12 membered heterocyclyl) is optionally substituted with one or more R₁₆ₒ;
Said 3-12 membered cycloalkyl in -L₅-(3-12 membered cycloalkyl) is optionally substituted with one or more R₁₆ₒ;
Said 6-12 member aryl in -L₅-(6-12 member aryl) is optionally substituted with one or more R₁₆ₒ;
Said 5-12 membered heteroaryl in -L₅-(5-12 membered heteroaryl) is optionally substituted with one or more R₁₆ₒ;
L₇ is selected from a bond or C₁₋₁₀ alkylene optionally substituted with one or more R_{16q};
L₈ is selected from a bond or C₁₋₁₀ alkylene optionally substituted with one or more R₁₆ᵣ;
Ring E is selected from a 3-10 membered carbocyclic ring or a 3-10 membered heterocyclic ring; wherein the moiety of -L₇- and -L₈-X₆ are attached to the same atom or different atoms of the ring E;
X₆ is selected from —N(R₆₅)₂, —OR₆₅, —SR₆₅, 3-10 membered heterocyclyl, or 5-10 membered heteroaryl, wherein said 3-10 membered heterocyclyl or 5-10 membered heteroaryl is optionally independently substituted with one or more R₁₆ₛ;
Y is selected from a bond, —C(R₆₃)₂—, —R₆₃C═CR₆₃—, —C≡C—, —C(═O)—, —O—, —NR₆₃—, —S—, —S(═O)—, —S(═O)₂—, —PR₆₃—, —P(═O)R₆₄—, —C(═O)O—, —OC(═O)—, —C(═O)NR₆₃—, —NR₆₃C(═O)—, —S(═O)O—, —OS(═O)—, —S(═O)₂O—, —OS(═O)₂—, —S(═O)NR₆₃—, —NR₆₃S(═O)—, —S(═O)₂NR₆₃—, —NR₆₃S(═O)₂—, —OC(═O)O—, —NR₆₃C(═O)O—, —OC(═O)NR₆₃— or —NR₆₃C(═O)NR₆₃—;
L₁ is selected from a bond, —C(R₆₅)₂—, —R₆₅C═CR₆₅—, —C≡C—, —C(═O)—, —O—, —NR₆₅—, —S—, —S(═O)—, —S(═O)₂—, —PR₆₅—, —P(═O)R₆₆—, —C(═O)O—, —OC(═O)—, —C(═O)NR₆₅—, —NR₆₅C(═O)—, —S(═O)O—, —OS(═O)—, —S(═O)₂O—, —OS(═O)₂—, —S(═O)NR₆₅—, —NR₆₅S(═O)—, —S(═O)₂NR₆₅—, —NR₆₅S(═O)₂—, —OC(═O)O—, —NR₆₅C(═O)O—, —OC(═O)NR₆₅— or —NR₆₅C(═O)NR₆₅—;
t is selected from 0, 1, 2, 3, 4, 5, 6, 7, 8, 9 or 10;
Ring A or ring B is a 3-20 membered heterocyclic ring which is optionally further contains 1, 2, 3, 4, 5, 6, 7, 8, 9, or 10 ring members selected from —N—, —O—, —S—, —C(═O)—, —S(═O)—, —S(═O)₂—, —S(═O)O—, —OS(═O)—, —S(═O)₂O—, —OS(═O)₂—, —S(═O)NH—, —NHS(═O)—, —S(═O)₂NH—, —NHS(═O)₂—; or a 3-20 membered heteroaryl ring which is optionally further contains 1, 2, 3, 4, 5, 6, 7, 8, 9, or 10 ring members, selected from —N—, —O— or —S—;
Each of R_{S2} at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₇)₂, —OR₆₇, —SR₆₇, —S(═O)R₆₈, —S(═O)₂R₆₈, —C(═O)R₆₈, —C(═O)OR₆₇, —OC(═O)R₆₈, —C(═O)N(R₆₇)₂, —NR₆₇C(═O)R₆₈, —OC(═O)OR₆₇, —NR₆₇C(═O)OR₆₇, —NR₆₇C(═S)OR₆₇, —OC(═O)N(R₆₇)₂, —NR₆₇C(═O)N(R₆₇)₂, —S(═O)OR₆₇, —OS(═O)R₆₇, —S(═O)N(R₆₇)₂, —NR₆₇S(═O)R₆₈, —S(═O)₂OR₆₇, —OS(═O)₂R₆₈, —S(═O)₂N(R₆₇)₂, —NR₆₇S(═O)₂R₆₈, —OS(═O)₂OR₆₇, —NR₆₇S(═O)₂OR₆₇, —OS(═O)₂N(R₆₇)₂, —NR₆₇S(═O)₂N(R₆₇)₂, —P(R₆₇)₂, —P(═O)(R₆₈)₂,

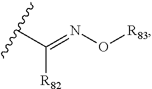

3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₇)₂, —OR₆₇, —SR₆₇, —S(═O)R₆₈, —S(═O)₂R₆₈, —C(═O)R₆₈, —C(═O)OR₆₇, —OC(═O)R₆₈, —C(═O)N(R₆₇)₂, —NR₆₇C(═O)R₆₈, —OC(═O)OR₆₇, —NR₆₇C(═O)OR₆₇, —NR₆₇C(═S)OR₆₇, —OC(═O)N(R₆₇)₂, —NR₆₇C(═O)N(R₆₇)₂, —S(═O)OR₆₇, —OS(═O)R₆₇, —S(═O)N(R₆₇)₂, —NR₆₇S(═O)R₆₈, —S(═O)₂OR₆₇, —OS(═O)₂R₆₈, —S(═O)₂N(R₆₇)₂, —NR₆₇S(═O)₂R₆₈, —OS(═O)₂OR₆₇, —NR₆₇S(═O)₂OR₆₇, —OS(═O)₂N(R₆₇)₂, —NR₆₇S(═O)₂N(R₆₇)₂, —P(R₆₇)₂, —P(═O)(R₆₈)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S2} together with the carbon atom to which they are both attached form

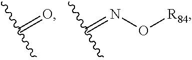

a 3-10 membered carbocyclic ring or a 3-10 heterocyclic ring; wherein, sais 3-10 membered carbocylic ring or 3-10 heterocyclic ring is optionally substituted with one or more R_{16d};
Optionally, two adjacent R_{S2} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₁₆ₑ;
Optionally, two nonadjacent R_{S2} are connected together to form a C₀₋₆ alkylene bridge, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R_{16f};
q₂ is selected from 0, 1, 2, 3, 4, 5, 6, 7, 8, 9 or 10;
Each of R_{S6} at each occurrence is independently selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₃)₂, —OR₇₃, —SR₇₃, —S(═O)R₇₄, —S(═O)₂R₇₄, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₃, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₃)₂, —OR₇₃, —SR₇₃, —S(═O)R₇₄, —S(═O)₂R₇₃, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₄, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
q₆ is selected from 0, 1, 2, 3, 4, 5 or 6;
R₄ is selected from 6-20 membered aryl, 5-20 membered heteroaryl,

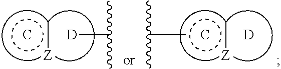

wherein said 6-20 membered aryl, 5-20 membered heteroaryl,

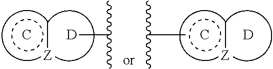

is optionally independently substituted with one or more R₄₁;
Z at each occurrence is independently selected from C or N;
Ring C at each occurrence is independently selected from a 6 membered aryl ring or a 5-6 membered heteroaryl ring and ring D at each occurrence is a 3-10 membered carbocyclic ring or a 3-10 membered heterocyclic ring when Z is selected from C;
Ring C at each occurrence is selected from a 5-6 membered heteroaryl ring and ring D at each occurrence is a 3-10 membered heterocyclic ring when Z is selected from N;
R₄₁ at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₉)₂, —OR₆₉, —SR₆₉, —S(═O)R₇₀, —S(═O)₂R₇₀, —C(═O)R₇₀, —C(═O)OR₆₉, —OC(═O)R₇₀, —C(═O)N(R₆₉)₂, —NR₆₉C(═O)R₇₀, —OC(═O)OR₆₉, —NR₆₉C(═O)OR₆₉, —OC(═O)N(R₆₉)₂, —NR₆₉C(═O)N(R₆₉)₂, —S(═O)OR₆₉, —OS(═O)R₇₀, —S(═O)N(R₆₉)₂, —NR₆₉S(═O)R₇₀, —S(═O)₂OR₆₉, —OS(═O)₂R₇₀, —S(═O)₂N(R₆₉)₂, —NR₆₉S(═O)₂R₇₀, —OS(═O)₂OR₆₉, —NR₆₉S(═O)₂OR₆₉, —OS(═O)₂N(R₆₉)₂, —NR₆₉S(═O)₂N(R₆₉)₂, —P(R₆₉)₂, —P(═O)(R₇₀)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₉)₂, —OR₆₉, —SR₆₉, —S(═O)R₇₀, —S(═O)₂R₇₀, —C(═O)R₇₀, —C(═O)OR₆₉, —OC(═O)R₇₀, —C(═O)N(R₆₉)₂, —NR₆₉C(═O)R₇₀, —OC(═O)OR₆₉, —NR₆₉C(═O)OR₆₉, —OC(═O)N(R₆₉)₂, —NR₆₉C(═O)N(R₆₉)₂, —S(═O)OR₆₉, —OS(═O)R₇₀, —S(═O)N(R₆₉)₂, —NR₆₉S(═O)R₇₀, —S(═O)₂OR₆₉, —OS(═O)₂R₇₀, —S(═O)₂N(R₆₉)₂, —NR₆₉S(═O)₂R₇₀, —OS(═O)₂OR₆₉, —NR₆₉S(═O)₂OR₆₉, —OS(═O)₂N(R₆₉)₂, —NR₆₉S(═O)₂N(R₆₉)₂, —P(R₆₉)₂, —P(═O)(R₇₀)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Each of (R₅₁, R₅₂ and R₅₃) is independently selected from hydrogen, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, —N(R₇₁)₂, —OR₇₁, —SR₇₁, —S(═O)R₇₂, —S(═O)₂R₇₂, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₁, —OS(═O)₂N(R₇₁)₂, —NR₇₁S(═O)₂N(R₇₁)₂, —P(R₇₁)₂, —P(═O)(R₇₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₇₁)₂, —OR₇₁, —SR₇₁, —S(═O)R₇₂, —S(═O)₂R₇₂, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₁, —OS(═O)₂N(R₇₁)₂, —NR₇₁S(═O)₂N(R₇₁)₂, —P(R₇₁)₂, —P(═O)(R₇₂)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₆₁, R₆₂, R₆₃, R₆₄, R₆₅, R₆₆, R₆₇, R₆₈, R₆₉, R₇₀, R₇₁, R₇₂, R₇₃, R₇₄, R₇₅, R₈₀, R₈₁, R₈₂, R₈₃, and R₈₄) at each occurrence is independently selected from hydrogen, —C₁₋₁₀alkyl, haloC₁₋₁₀alkyl, haloC₁₋₁₀alkoxy, —C₂₋₁₀alkenyl, —C₂₋₁₀alkynyl, —CN, —NO₂, —N₃, oxo, —N(Rₐ)₂, —ORₐ, —SRₐ, —S(═O)R_{b}, —S(═O)₂R_{b}, —C(═O)R_{b}, —C(═O)ORₐ, —OC(═O)R_{b}, —C(═O)N(Rₐ)₂, —NRₐC(═O)R_{b}, —OC(═O)ORₐ, —NR_{c}C(═O)ORₐ, —OC(═O)N(Rₐ)₂, —NRₐC(═O)N(Rₐ)₂, —S(═O)ORₐ, —OS(═O)R_{b}, —S(═O)N(Rₐ)₂, —NRₐS(═O)R_{b}, —S(═O)₂ORₐ, —OS(═O)₂R_{b}, —S(═O)₂N(Rₐ)₂, —NRₐS(═O)₂R_{b}, —OS(═O)₂ORₐ, —NRₐS(═O)₂ORₐ, —OS(═O)₂NRₐ, —NRₐS(═O)₂N(Rₐ)₂, —P(Rₐ)₂, —P(═O)(R_{b})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR_{c}, —SR_{c}, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, each of (two R₆₁, two R₆₇, two R₆₉, two R₇₁, two R₇₃, two R₇₅, two Rₐ and two R_{c}) independently together with the nitrogen atom to which they are both attached forms a 3-20 membered heterocyclic ring or a 5-10 membered heteroaryl ring, wherein, said 3-20 membered heterocyclic ring or 5-10 membered heteroaryl ring is optionally independently substituted with one or more R_{16g};
Optionally, each of (two R₆₃ and two R₆₅) independently together with the carbon atom to which they are both or independently attached forms a 3-20 membered carbocyclic ring or 3-20 membered heterocyclic ring, wherein, said 3-20 membered carbocyclic ring or 3-20 membered heterocyclic ring is optionally independently substituted with one or more R₁₆ₕ;
Each of (Rₐ, R_{b}, R_{c} and R_{d}) at each occurrence is independently selected from hydrogen, —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₁₆;
Each of (R₁₆ₐ, R_{16b}, R_{16c}, R_{16d}, R₁₆ₑ, R_{16f}, R_{16g}, R₁₆ₕ, R₁₆ᵢ, R₁₆ₙ, R₁₆ₒ, R_{16q}, R₁₆ᵣ and R₁₆ₛ) at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

[2]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1], wherein, the compound is of formula (I-1):

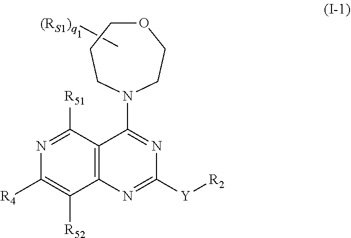

Wherein,
R₂ is selected from

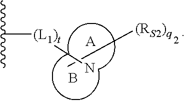

[3]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1] or [2], wherein,
R_{S1} at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, —NH₂, —NH(C₁₋₆alkyl), —N(C₁₋₆alkyl)₂, —NH(3-10 membered cycloalkyl), —N(C₁₋₆alkyl)(3-10 membered cycloalkyl), —OH, —O(C₁₋₆alkyl), —O-(3-10 membered cycloalkyl), —SH, —S(C₁₋₆alkyl), —S(3-10 membered cycloalkyl), —S(═O)(C₁₋₆alkyl), —S(═O)(3-10 membered cycloalkyl), —S(═O)₂(C₁₋₆alkyl), —S(═O)₂(3-10 membered cycloalkyl), —C(═O)(C₁₋₆alkyl), —C(═O)-(3-10 membered cycloalkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —OS(═O)O(C₁₋₆alkyl), —NHS(═O)O(C₁₋₆alkyl), —N(C₁₋₆alkyl)S(═O)O(C₁₋₆alkyl), —OS(═O)NH₂, —OS(═O)NH(C₁₋₆alkyl), —OS(═O)N(C₁₋₆alkyl)₂, —NHS(═O)NH₂, —NHS(═O)NH(C₁₋₆alkyl), —NHS(═O)N(C₁₋₆alkyl)₂, —N(C₁₋₆alkyl)S(═O)NH₂, —N(C₁₋₆alkyl)S(═O)NH(C₁₋₆alkyl), —N(C₁₋₆alkyl)S(═O)N(C₁₋₆alkyl)₂, —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered heterocyclyl, 6-10 membered aryl or 5-10 membered heteroaryl, wherein said —C₁₋₆alkyl, —C₁₋₆haloalkyl, —C₁₋₆haloalkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —NH(3-10 membered cycloalkyl), —N(C₁₋₆alkyl)(3-10 membered cycloalkyl), —OH, —O(C₁₋₆alkyl), —O(3-10 membered cycloalkyl), —SH, —S(C₁₋₆alkyl), —S(3-10 membered cycloalkyl), —S(═O)(C₁₋₆-alkyl), —S(═O)(3-10 membered cycloalkyl), —S(═O)₂(C₁₋₆alkyl), —S(═O)₂(3-10 membered cycloalkyl), —C(═O)(C₁₋₆alkyl), —C(═O)-(3-10 membered cycloalkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —OS(═O)O(C₁₋₆alkyl), —NHS(═O)O(C₁₋₆alkyl), —N(C₁₋₆alkyl)S(═O)O(C₁₋₆alkyl), —OS(═O)NH₂, —OS(═O)NH(C₁₋₆alkyl), —OS(═O)N(C₁₋₆alkyl)₂, —NHS(═O)NH₂, —NHS(═O)NH(C₁₋₆alkyl), —NHS(═O)N(C₁₋₆alkyl)₂, —N(C₁₋₆alkyl)S(═O)NH₂, —N(C₁₋₆alkyl)S(═O)NH(C₁₋₆alkyl), —N(C₁₋₆alkyl)S(═O)N(C₁₋₆alkyl)₂, —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered heterocyclyl, 6-10 membered aryl or 5-10 membered heteroaryl;

[4]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [3], wherein,
R_{S1} at each occurrence is independently selected from —F, —Cl, —Br, —C₁₋₃alkyl, —C₁₋₃haloalkyl, —C₁₋₃haloalkoxy, —C₂₋₃alkenyl, —C₂₋₃alkynyl, —CN, —NH₂, —NH(C₁₋₃alkyl), —N(C₁₋₃alkyl)₂, —NH(3-6 membered cycloalkyl), —N(C₁₋₃alkyl)(3-6 membered cycloalkyl), —OH, —O(C₁₋₃alkyl), —O-(3-6 membered cycloalkyl), —SH, —S(C₁₋₃alkyl), —S-(3-6 membered cycloalkyl), —S(═O)(C₁₋₃alkyl), —S(═O)(3-6 membered cycloalkyl), —S(═O)₂(C₁₋₃alkyl), —S(═O)₂-(3-6 membered cycloalkyl), —C(═O)(C₁₋₃alkyl), —C(═O)-(3-6 membered cycloalkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered heterocyclyl, 6-10 membered aryl or 5-10 membered heteroaryl, wherein said —C₁₋₃alkyl, —C₁₋₃haloalkyl, —C₁₋₃haloalkoxy, —C₂₋₆alkenyl, —C₂₋₆alkynyl, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —C₁₋₃alkyl, —C₁₋₃haloalkyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂ or 3-6 membered cycloalkyl.

[5]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomcr, a prodrug thereof, a deuterated molecule thereof or a conjugated form thereof of any one of [1] to [4], wherein,
R_{S1} at each occurrence is independently selected from —Cl, —F, —Br, —CH₃, —CH₂CH₃, —CH₂CH₂CH₃, —CH(CH₃)₂, —C(CH₃)₃,

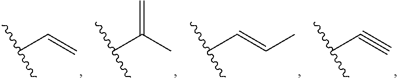

—CH₂F, —CHF₂, —CF₃, —CH₂CH₂F, —CH₂CHF₂, —CH₂CF₃, —CHFCH₃, —CF₂CH₃, —CN, —NH₂, —NH(CH₃), —N(CH₃)₂, —NH(CH₂CH₃), —OH, —O—CH₃, —O—CH₂CH₃, —O—CH₂CH₂CH₃, —O—CH(CH₃)₂, —O—CF₃, —SH, —S—CH₃, —S—CH₂CH₃, —S—CH₂CH₂CH₃, —S—CH(CH₃)₂, —S—CF₃, —S(═O)CH₃, —S(═O)(CH₂CH₃), —S(═O)(CH₂CH₂CH₃), —S(═O)(CH(CH₃)₂), —S(═O)₂CH₃, —S(═O)₂(CH₂CH₃), —S(═O)₂(CH₂CH₂CH₃), —S(═O)₂(CH(CH₃)₂), —COOH, —C(═O)(CH₃), —C(═O)(CH₂CH₃), —C(═O)(CH(CH₃)₂), —C(═O)(CF₃), —C(═O)(OCH₃), —C(═O)(OCH₂CH₃), —C(═O)(OCH₂CH₂CH₃), —C(═O)(OCH(CH₃)₂), —OC(═O)(CH₃), —OC(═O)(CH₂CH₃), —OC(═O)(CH₂CH₂CH₃), —OC(═O)(CH(CH₃)₂), —C(═O)NH₂, —C(═O)NH(CH₃), —C(═O)NH(CH₂CH₃), —C(═O)NH(CH₂CH₂CH₃), —C(═O)NH(CH(CH₃)₂), —C(═O)N(CH₃)₂, —C(═O)N(CH₂CH₃)₂, —NHC(═O)(CH₃), —NHC(═O)(CH₂CH₃), —NHC(═O)(CH₂CH₂CH₃), —NHC(═O)(CH(CH₃)₂), —N(CH₃)C(═O)(CH₃), —S(═O)(OCH₃), —S(═O)(OCH₂CH₃), —S(═O)(OCH₂CH₂CH₃), —S(═O)(OCH(CH₃)₂), —OS(═O)(CH₃), —OS(═O)(CH₂CH₃), —OS(═O)(CH₂CH₂CH₃), —OS(═O)(CH(CH₃)₂), —S(═O)NH₂, —S(═O)NH(CH₃), —S(═O)NH(CH₂CH₃), —S(═O)NH(CH₂CH₂CH₃), —S(═O)NH(CH(CH₃)₂), —S(═O)N(CH₃)₂, —S(═O)N(CH₃)(CH₂CH₃), —NHS(═O)(CH₃), —NHS(═O)(CH₂CH₃), —NHS(═O)(CH₂CH₂CH₃), —NHS(═O)(CH(CH₃)₂), —N(CH₃)S(═O)(CH₃), —S(═O)₂(OCH₃), —S(═O)₂(OCH₂CH₃), —S(═O)₂(OCH₂CH₂CH₃), —S(═O)₂(OCH(CH₃)₂), —OS(═O)₂(CH₃), —OS(═O)₂(CH₂CH₃), —OS(═O)₂(CH₂CH₂CH₃), —OS(═O)₂(CH(CH₃)₂), —S(═O)₂NH₂, —S(═O)₂NH(CH₃), —S(═O)₂NH(CH₂CH₃), —S(═O)₂NH(CH₂CH₂CH₃), —S(═O)₂NH(CH(CH₃)₂), —S(═O)₂N(CH₃)₂, —S(═O)₂N(CH₃)(CH₂CH₃), —NHS(═O)₂(CH₃), —NHS(═O)₂(CH₂CH₃), —NHS(═O)₂(CH₂CH₂CH₃), —NHS(═O)₂(CH(CH₃)₂), —N(CH₃)S(═O)₂(CH₃), —P(═O)H(CH₃), —P(═O)H(CH₂CH₃), —P(═O)H(CH₂CH₂CH₃), —P(═O)H(CH(CH₃)₂), —P(═O)(CH₃)₂, —P(═O)(CH₃)(CH₂CH₃), —CH₂—OH, —CH₂CH₂—OH, —CH(CH₃)—OH, —CH₂—SH, —CH₂CH₂—SH, —CH(CH₃)—SH, —CH₂—NH₂, —CH₂CH₂—NH₂, —CH(CH₃)—NH₂, —CH₂—CN, —CH₂CH₂—CN, —CH(CH₃)—CN, —O—CH₂—O—CH₃, —O—CH₂CH₃—O—CH₃, —O—CH(CH₃)—O—CH₃, —O—CH₂CH₂CH₃—O—CH₃, —O—CH₂CH(CH₃)—O—CH₃, —O—CH(CH₃)CH₂—O—CH₃, —NH—O—CH₃, —N(CH₃)—O—CH₃, —N(CH₂CH₃)—O—CH₃,

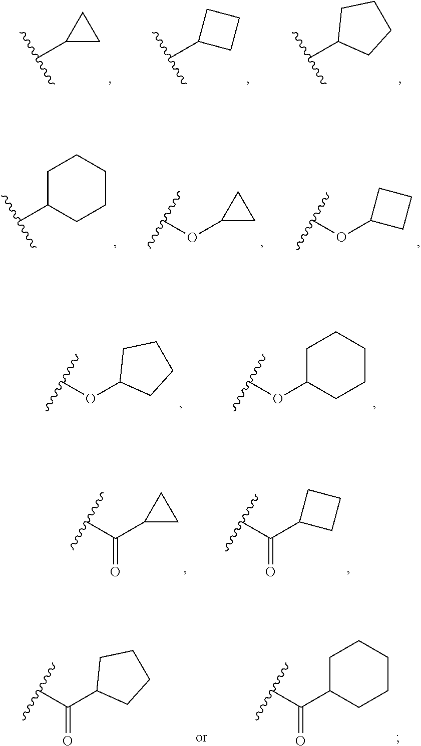

Optionally, two R_{S1} together with the carbon atom to which they are both attached form

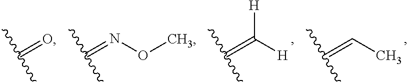

a 3 membered carbocyclic ring, or a 4 membered carbocyclic ring.

[6]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [5], wherein,
R_{S1} at each occurrence is independently selected from —Cl, —F, —Br, —CH₃, —CH₂CH₃, —CH(CH₃)₂, —C(CH₃)₃,

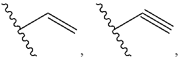

—CH₂F, —CHF₂, —CF₃, —CHFCH₃, —CF₂CH₃, —CN, —NH₂, —NH(CH₃), —N(CH₃)₂, —OH, —O—CH₃, —O—CH₂CH₃, —O—CH(CH₃)₂, —C(═O)CH₃, —C(═O)(CH₂CH₃), —C(═O)—CH(CH₃)₂, —C(═O)(CF₃), —C(═O)NH₂, —C(═O)NH(CH₃), —CH₂—OH, —CH₂CH₂—OH, —CH(CH₃)—OH, —CH₂—CN, —CH₂CH₂—CN, —CH(CH₃—CN, —O—CH₃—O—CH₃, —O—CH₂CH₃—O—CH₃, —N(CH₃)—O—CH₃, —N(CH₂CH₃)—O—CH₃,

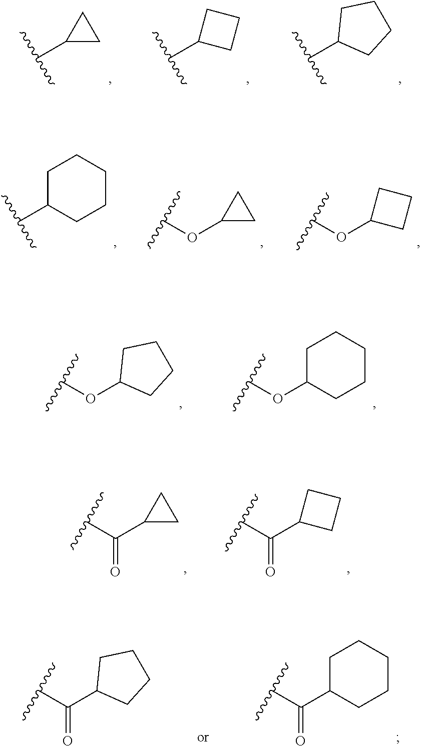

Optionally two R_{S1} together with the carbon atom to which they are both attached form

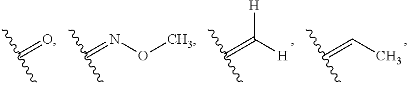

a 3 membered carbocyclic ring, or a 4 membered carbocyclic ring. [7]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [6], wherein,
R_{S1} at each occurrence is independently selected from —Cl, —F, —Br, —CH₃, —CH₂CH₃, —CH(CH₃)₂, —C(CH₃)₃, —CN, —CH₂—CN, —CH₂CH₂—CN or —CH(CH₃)—CN.

[8]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1] or [2], wherein, two adjacent R_{S1} together with the carbon atoms to which they are respectively attached form a 3-7 membered carbocyclic ring; a 3-7 membered heterocyclic ring; a 6-12 membered aryl ring or a 5-10 membered heteroaryl ring; said heterocyclic ring and heteroaryl ring are independently contains 0, 1, 2, 3, 4, 5 or 6 ring members selected from N, O or S.

[9]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [8], wherein, two adjacent R_{S1} together with the carbon atoms to which they are respectively attached form a 3 membered carbocyclic ring; 4 membered carbocyclic ring; 5 membered carbocyclic ring; 6 membered carbocyclic ring; 7 membered carbocyclic ring; 3 membered heterocyclic ring; 4 membered heterocyclic ring; 5 membered heterocyclic ring; 6 membered heterocyclic ring; 7 membered heterocyclic ring; benzene ring; naphthalene ring; 5 membered heteroaryl ring or 6 membered heteroaryl ring; said heterocyclic ring and heteroaryl ring are independently contains 0, 1, 2, or 3 ring members selected from N, O or S.

[10]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [9], wherein, two adjacent R_{S1} together with the carbon atoms to which they are respectively attached form a 3 membered carbocyclic ring; 4 membered carbocyclic ring or 6 membered heterocyclic ring containing 2 ring members selected from O.

[11]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [10], wherein, the moiety of

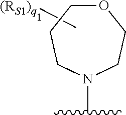

is selected from

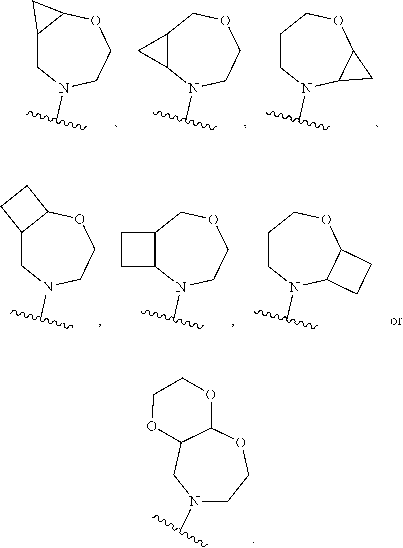

[12]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [11], wherein, the moiety of

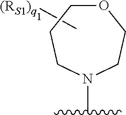

is selected from any one in the Table 1:

TABLE 1

| 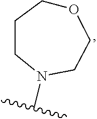 [SMILES: *N1CCCOCC1] | 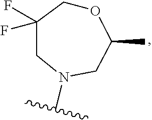 [SMILES: *N1C[C@H](C)OCC(F)(F)C1] |
|---|---|
| 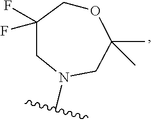 [SMILES: *N1CC(F)(F)COC(C)(C)C1] | 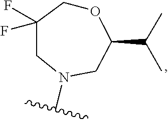 [SMILES: *N1C[C@H](C(C)C)OCC(F)(F)C1] |
| 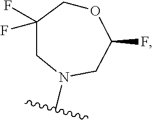 [SMILES: *N1C[C@H](F)OCC(F)(F)C1] | 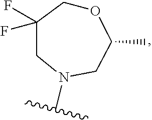 [SMILES: *N1C[C@@H](C)OCC(F)(F)C1] |
| 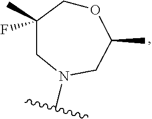 [SMILES: *N1C[C@H](C)OC[C@@](C)(F)C1] | 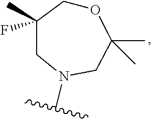 [SMILES: *N1CC(C)(C)OC[C@@](C)(F)C1] |
| 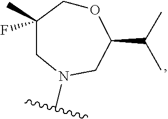 [SMILES: *N1C[C@H](C(C)C)OC[C@@](C)(F)C1] | 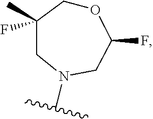 [SMILES: *N1C[C@H](F)OC[C@@](C)(F)C1] |
| 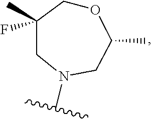 [SMILES: *N1C[C@@H](C)OC[C@@](C)(F)C1] | 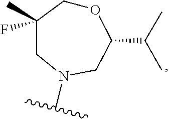 [SMILES: *N1C[C@@H](C(C)C)OC[C@@](C)(F)C1] |
| 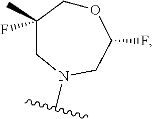 [SMILES: *N1C[C@@H](F)OC[C@@](C)(F)C1] | 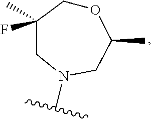 [SMILES: *N1C[C@H](C)OC[C@](C)(F)C1] |
| 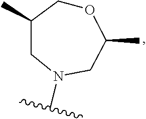 | 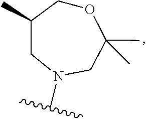 |
| 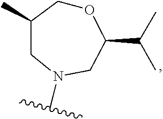 [SMILES: *N1C[C@@H](C)CO[C@@H](C(C)C)C1] | 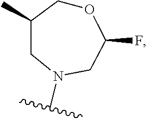 [SMILES: *N1C[C@@H](C)CO[C@@H](F)C1] |
| 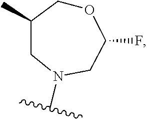 [SMILES: *N1C[C@@H](C)CO[C@H](F)C1] | 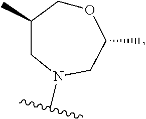 |
| 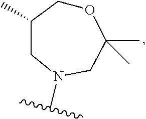 | 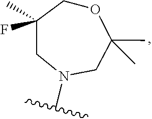 [SMILES: *N1CC(C)(C)OC[C@](C)(F)C1] |
| 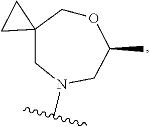 [SMILES: *N1C[C@H](C)OCC2(CC2)C1] | 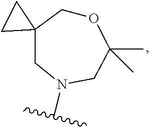 [SMILES: *N1CC2(CC2)COC(C)(C)C1] |
| 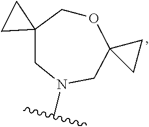 [SMILES: *N1CC2(CC2)COC2(CC2)C1] | 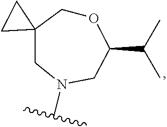 [SMILES: *N1C[C@H](C(C)C)OCC2(CC2)C1] |
| 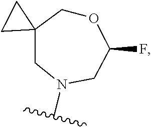 [SMILES: *N1C[C@H](F)OCC2(CC2)C1] | 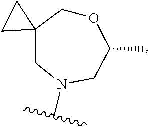 [SMILES: *N1C[C@@H](C)OCC2(CC2)C1] |
| 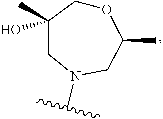 [SMILES: *N1C[C@H](C)OC[C@@](C)(O)C1] | 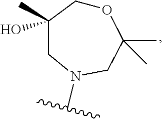 [SMILES: *N1CC(C)(C)OC[C@@](C)(O)C1] |
| 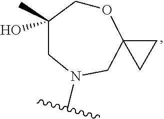 [SMILES: *N1CC2(CC2)OC[C@@](C)(O)C1] | 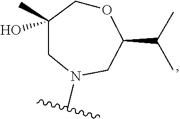 [SMILES: *N1C[C@H](C(C)C)OC[C@@](C)(O)C1] |
| 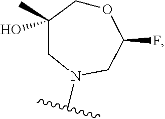 [SMILES: *N1C[C@H](F)OC[C@@](C)(O)C1] | 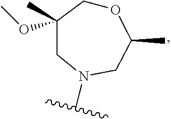 |
| 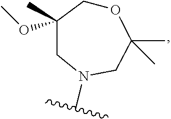 | 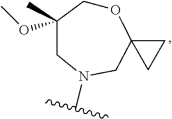 [SMILES: *N1CC2(CC2)OC[C@@](C)(OC)C1] |
| 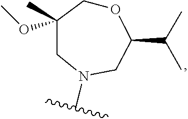 [SMILES: *N1C[C@H](C(C)C)OC[C@@](C)(OC)C1] | |
| 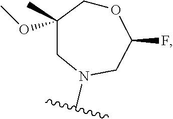 [SMILES: *N1C[C@H](F)OC[C@@](C)(OC)C1] | 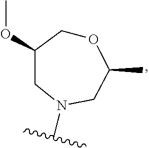 [SMILES: *N1C[C@@H](OC)CO[C@@H](C)C1] |
| 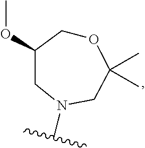 [SMILES: *N1C[C@@H](OC)COC(C)(C)C1] | 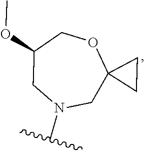 [SMILES: *N1C[C@@H](OC)COC2(CC2)C1] |
| 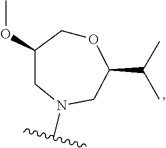 [SMILES: *N1C[C@@H](OC)CO[C@@H](C(C)C)C1] | 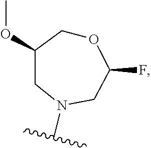 [SMILES: *N1C[C@@H](OC)CO[C@@H](F)C1] |
| 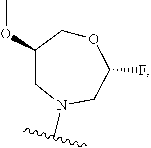 [SMILES: *N1C[C@@H](OC)CO[C@H](F)C1] | 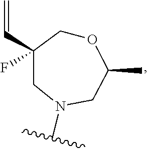 [SMILES: *N1C[C@H](C)OC[C@](F)(C=C)C1] |
| 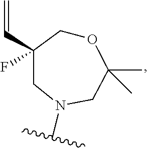 [SMILES: *N1CC(C)(C)OC[C@](F)(C=C)C1] | 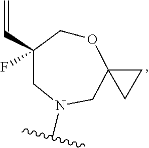 [SMILES: *N1CC2(CC2)OC[C@](F)(C=C)C1] |
| 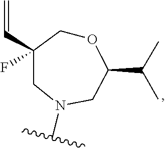 [SMILES: *N1C[C@H](C(C)C)OC[C@](F)(C=C)C1] | 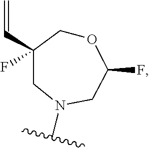 [SMILES: *N1C[C@H](F)OC[C@](F)(C=C)C1] |
| 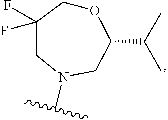 [SMILES: *N1C[C@@H](C(C)C)OCC(F)(F)C1] | 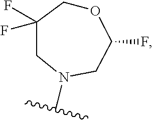 [SMILES: *N1C[C@@H](F)OCC(F)(F)C1] |
| 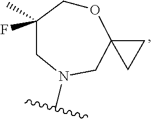 [SMILES: *N1CC2(CC2)OC[C@](C)(F)C1] | 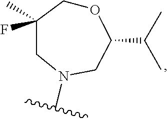 [SMILES: *N1C[C@@H](C(C)C)OC[C@](C)(F)C1] |
| 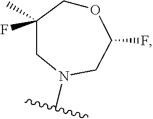 [SMILES: *N1C[C@@H](F)OC[C@](C)(F)C1] | 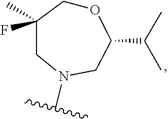 [SMILES: *N1C[C@@H](C(C)C)OC[C@](C)(F)C1] |
| 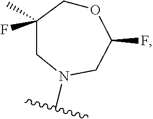 [SMILES: *N1C[C@H](F)OC[C@](C)(F)C1] | 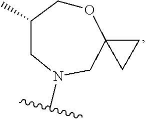 [SMILES: *N1C[C@H](C)COC2(CC2)C1] |
| 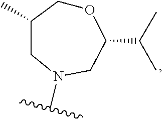 [SMILES: *N1C[C@H](C)CO[C@H](C(C)C)C1] | 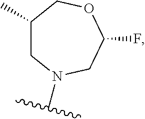 [SMILES: *N1C[C@H](C)CO[C@H](F)C1] |
| 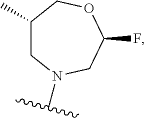 [SMILES: *N1C[C@H](C)CO[C@@H](F)C1] | 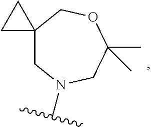 [SMILES: *N1CC2(CC2)COC(C)(C)C1] |
| 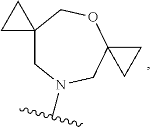 [SMILES: *N1CC2(CC2)COC2(CC2)C1] | 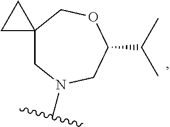 [SMILES: *N1C[C@@H](C(C)C)OCC2(CC2)C1] |
| 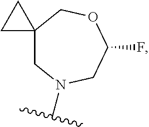 [SMILES: *N1C[C@@H](F)OCC2(CC2)C1] | 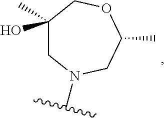 [SMILES: *N1C[C@@H](C)OC[C@](C)(O)C1] |
| 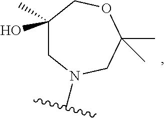 [SMILES: *N1CC(C)(C)OC[C@](C)(O)C1] | 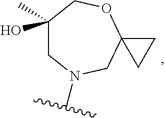 [SMILES: *N1CC2(CC2)OC[C@](C)(O)C1] |
| 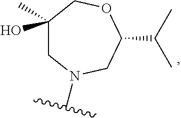 [SMILES: *N1C[C@@H](C(C)C)OC[C@](C)(O)C1] | 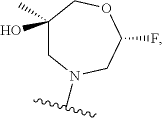 [SMILES: *N1C[C@@H](F)OC[C@](C)(O)C1] |
| 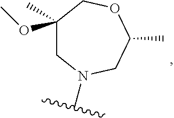 | 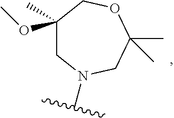 |
| 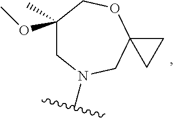 [SMILES: *N1CC2(CC2)OC[C@](C)(OC)C1] | 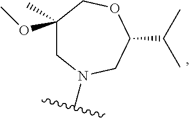 [SMILES: *N1C[C@@H](C(C)C)OC[C@](C)(OC)C1] |
| 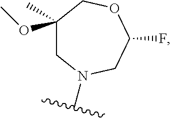 [SMILES: *N1C[C@@H](F)OC[C@](C)(OC)C1] | 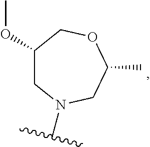 [SMILES: *N1C[C@H](OC)CO[C@H](C)C1] |
| 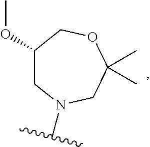 [SMILES: *N1C[C@H](OC)COC(C)(C)C1] | 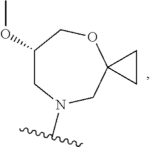 [SMILES: *N1C[C@H](OC)COC2(CC2)C1] |
| 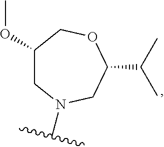 [SMILES: *N1C[C@H](OC)CO[C@H](C(C)C)C1] | 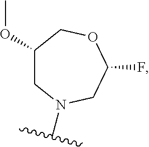 [SMILES: *N1C[C@H](OC)CO[C@H](F)C1] |
| 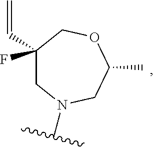 [SMILES: *N1C[C@@H](C)OC[C@@](F)(C=C)C1] | 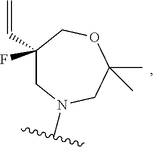 [SMILES: *N1CC(C)(C)OC[C@@](F)(C=C)C1] |
| 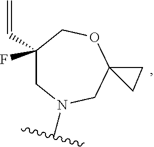 [SMILES: *N1CC2(CC2)OC[C@@](F)(C=C)C1] | 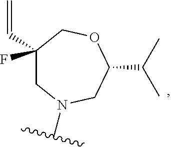 [SMILES: *N1C[C@@H](C(C)C)OC[C@@](F)(C=C)C1] |
| 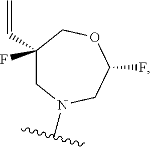 [SMILES: *N1C[C@@H](F)OC[C@@](F)(C=C)C1] | 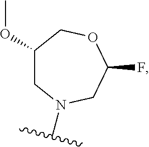 [SMILES: *N1C[C@H](OC)CO[C@@H](F)C1] |
| 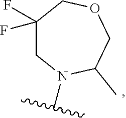 [SMILES: *N1CC(F)(F)COCC1C] | 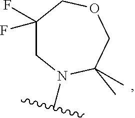 [SMILES: *N1CC(F)(F)COCC1(C)C] |
| 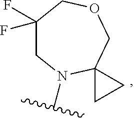 [SMILES: *N1CC(F)(F)COCC12CC2] | 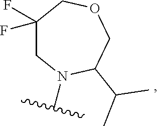 [SMILES: *N1CC(F)(F)COCC1C(C)C] |
| 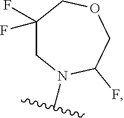 [SMILES: *N1CC(F)(F)COCC1F] | 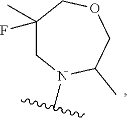 [SMILES: *N1CC(C)(F)COCC1C] |
| 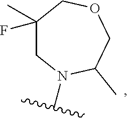 [SMILES: *N1CC(C)(F)COCC1C] | 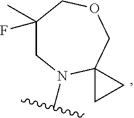 [SMILES: *N1CC(C)(F)COCC12CC2] |
| 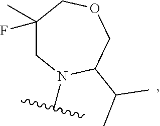 [SMILES: *N1CC(C)(F)COCC1C(C)C] | 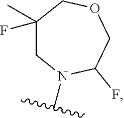 [SMILES: *N1CC(C)(F)COCC1F] |
| 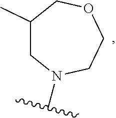 [SMILES: *N1CCOCC(C)C1] | 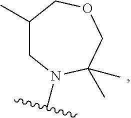 |
| 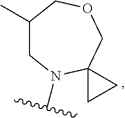 [SMILES: *N1CC(C)COCC12CC2] | 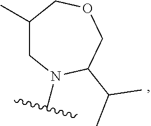 [SMILES: *N1CC(C)COCC1C(C)C] |
| 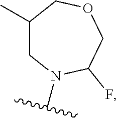 [SMILES: *N1CC(C)COCC1F] | 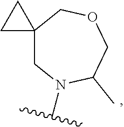 [SMILES: *N1CC2(CC2)COCC1C] |
| 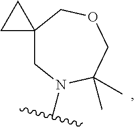 [SMILES: *N1CC2(CC2)COCC1(C)C] | 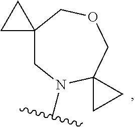 [SMILES: *N1CC2(CC2)COCC12CC2] |
| 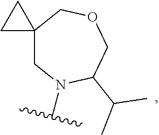 [SMILES: *N1CC2(CC2)COCC1C(C)C] | 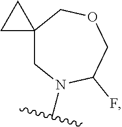 [SMILES: *N1CC2(CC2)COCC1F] |
| 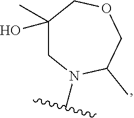 [SMILES: *N1CC(C)(O)COCC1C] | 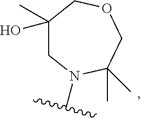 [SMILES: *N1CC(C)(O)COCC1(C)C] |
| 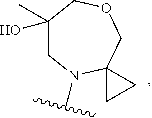 [SMILES: *N1CC(C)(O)COCC12CC2] | 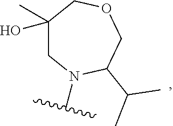 [SMILES: *N1CC(C)(O)COCC1C(C)C] |
| 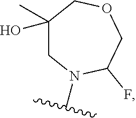 [SMILES: *N1CC(C)(O)COCC1F] | 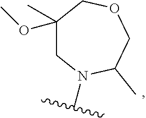 |
| 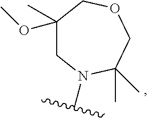 | 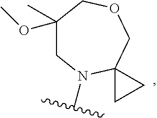 [SMILES: *N1CC(C)(OC)COCC12CC2] |
| 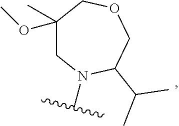 [SMILES: *N1CC(C)(OC)COCC1C(C)C] | 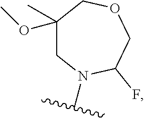 [SMILES: *N1CC(C)(OC)COCC1F] |
| 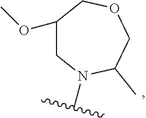 | 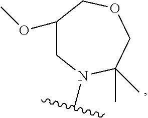 |
| 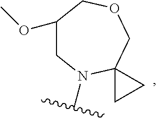 [SMILES: *N1CC(OC)COCC12CC2] | 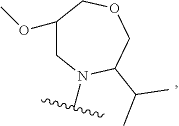 [SMILES: *N1CC(OC)COCC1C(C)C] |
| 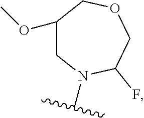 [SMILES: *N1CC(OC)COCC1F] | 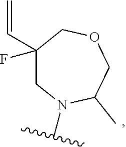 [SMILES: *N1CC(F)(C=C)COCC1C] |
| 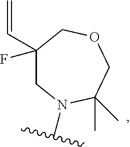 [SMILES: *N1CC(F)(C=C)COCC1(C)C] | 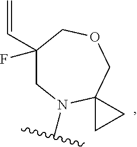 [SMILES: *N1CC(F)(C=C)COCC12CC2] |
| 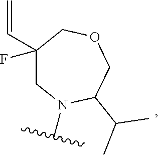 [SMILES: *N1CC(F)(C=C)COCC1C(C)C] | 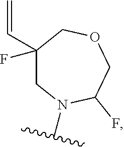 [SMILES: *N1CC(F)(C=C)COCC1F] |
| 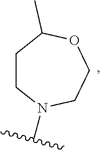 [SMILES: *N1CCOC(C)CC1] | 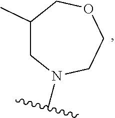 [SMILES: *N1CCOCC(C)C1] |
| 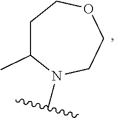 [SMILES: *N1CCOCCC1C] | 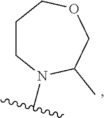 [SMILES: *N1CCCOCC1C] |
| 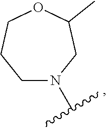 [SMILES: *N1CCCOC(C)C1] | 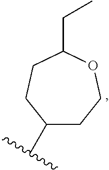 [SMILES: *C1CCOC(CC)CC1] |
| 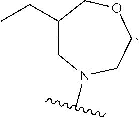 [SMILES: *N1CCOCC(CC)C1] | 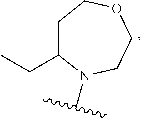 [SMILES: *N1CCOCCC1CC] |
| 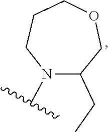 [SMILES: *N1CCCOCC1CC] | 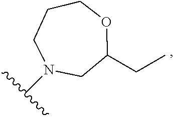 [SMILES: *N1CCCOC(CC)C1] |
| 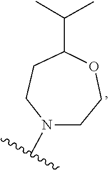 [SMILES: *N1CCOC(C(C)C)CC1] | 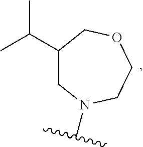 [SMILES: *N1CCOCC(C(C)C)C1] |
| 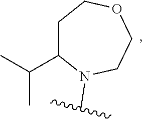 [SMILES: *N1CCOCCC1C(C)C] | 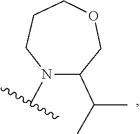 [SMILES: *N1CCCOCC1C(C)C] |
| 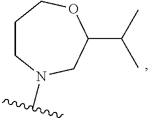 [SMILES: *N1CCCOC(C(C)C)C1] | 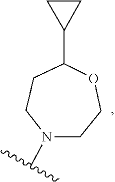 [SMILES: *N1CCOC(C2CC2)CC1] |
| 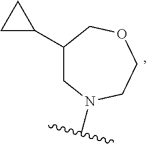 [SMILES: *N1CCOCC(C2CC2)C1] | 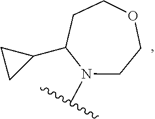 [SMILES: *N1CCOCCC1C1CC1] |
| 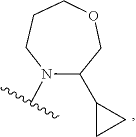 [SMILES: *N1CCCOCC1C1CC1] | 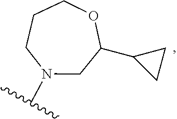 [SMILES: *N1CCCOC(C2CC2)C1] |
| 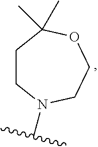 [SMILES: *N1CCOC(C)(C)CC1] | 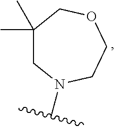 [SMILES: *N1CCOCC(C)(C)C1] |
| 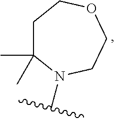 [SMILES: *N1CCOCCC1(C)C] | 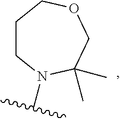 [SMILES: *N1CCCOCC1(C)C] |
| 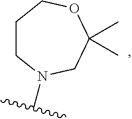 [SMILES: *N1CCCOC(C)(C)C1] | 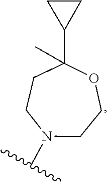 [SMILES: *N1CCOC(C)(C2CC2)CC1] |
| 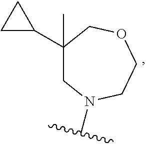 [SMILES: *N1CCOCC(C)(C2CC2)C1] | 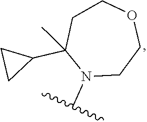 [SMILES: *N1CCOCCC1(C)C1CC1] |
| 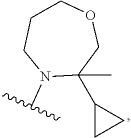 [SMILES: *N1CCCOCC1(C)C1CC1] | 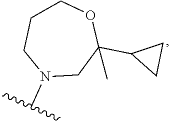 [SMILES: *N1CCCOC(C)(C2CC2)C1] |
| 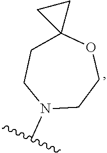 [SMILES: *N1CCOC2(CC1)CC2] | 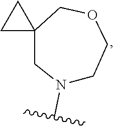 [SMILES: *N1CCOCC2(CC2)C1] |
| 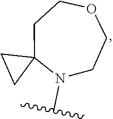 [SMILES: *N1CCOCCC12CC2] | 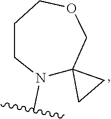 [SMILES: *N1CCCOCC12CC2] |
| 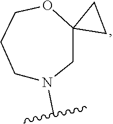 [SMILES: *N1CCCOC2(CC2)C1] | 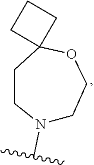 [SMILES: *N1CCOC2(CCC2)CC1] |
| 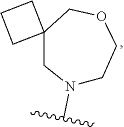 [SMILES: *N1CCOCC2(CCC2)C1] | 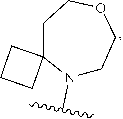 [SMILES: *N1CCOCCC12CCC2] |
| 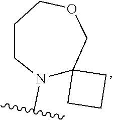 [SMILES: *N1CCCOCC12CCC2] | 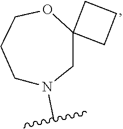 [SMILES: *N1CCCOC2(CCC2)C1] |
| 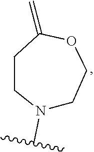 [SMILES: *N1CCOC(=C)CC1] | 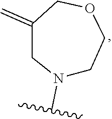 [SMILES: *N1CCOCC(=C)C1] |
| 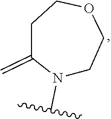 [SMILES: *N1CCOCCC1=C] | 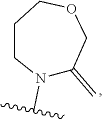 [SMILES: *N1CCCOCC1=C] |
| 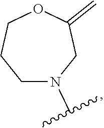 [SMILES: *N1CCCOC(=C)C1] | 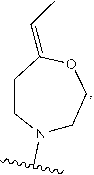 [SMILES: *N1CCO/C(=C\C)CC1] |
| 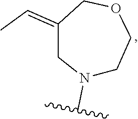 [SMILES: *N1CCOC/C(=C/C)C1] | 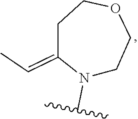 [SMILES: *N1CCOCC/C1=C\C] |
| 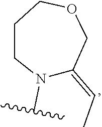 [SMILES: *N1CCCOC/C1=C/C] | 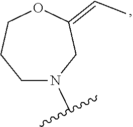 [SMILES: *N1CCCO/C(=C/C)C1] |
| 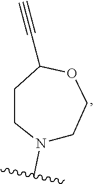 [SMILES: *N1CCOC(C#C)CC1] | 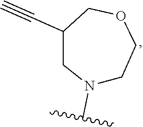 [SMILES: *N1CCOCC(C#C)C1] |
| 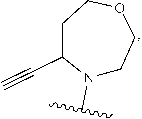 [SMILES: *N1CCOCCC1C#C] | 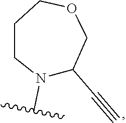 [SMILES: *N1CCCOCC1C#C] |
| 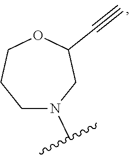 [SMILES: *N1CCCOC(C#C)C1] | 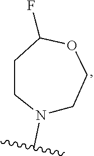 [SMILES: *N1CCOC(F)CC1] |
| 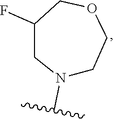 [SMILES: *N1CCOCC(F)C1] | 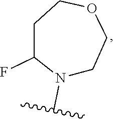 [SMILES: *N1CCOCCC1F] |
| 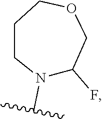 [SMILES: *N1CCCOCC1F] | 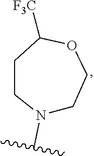 [SMILES: *N1CCOC(C(F)(F)F)CC1] |
| 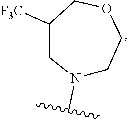 [SMILES: *N1CCOCC(C(F)(F)F)C1] | 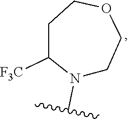 [SMILES: *N1CCOCCC1C(F)(F)F] |
| 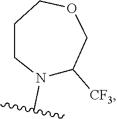 [SMILES: *N1CCCOCC1C(F)(F)F] | 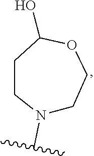 [SMILES: *N1CCOC(O)CC1] |
| 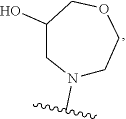 [SMILES: *N1CCOCC(O)C1] | 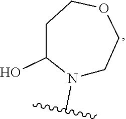 [SMILES: *N1CCOCCC1O] |
| 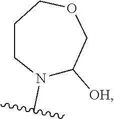 [SMILES: *N1CCCOCC1O] | 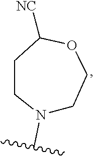 [SMILES: *N1CCOC(C#N)CC1] |
| 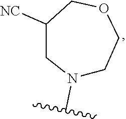 [SMILES: *N1CCOCC(C#N)C1] | 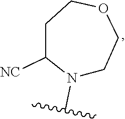 [SMILES: *N1CCOCCC1C#N] |
| 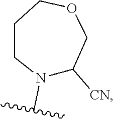 [SMILES: *N1CCCOCC1C#N] | 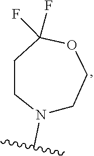 [SMILES: *N1CCOC(F)(F)CC1] |
| 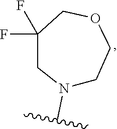 [SMILES: *N1CCOCC(F)(F)C1] | 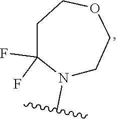 [SMILES: *N1CCOCCC1(F)F] |
| 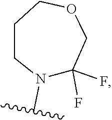 [SMILES: *N1CCCOCC1(F)F] | 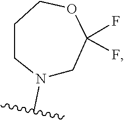 [SMILES: *N1CCCOC(F)(F)C1] |
| 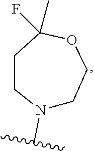 [SMILES: *N1CCOC(C)(F)CC1] | 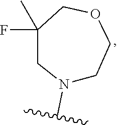 [SMILES: *N1CCOCC(C)(F)C1] |
| 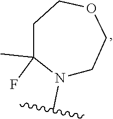 [SMILES: *N1CCOCCC1(C)F] | 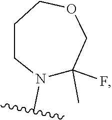 [SMILES: *N1CCCOCC1(C)F] |
| 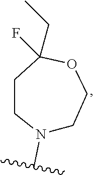 [SMILES: *N1CCOC(F)(CC)CC1] | 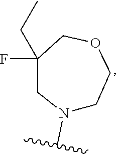 [SMILES: *N1CCOCC(F)(CC)C1] |
| 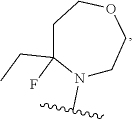 [SMILES: *N1CCOCCC1(F)CC] | 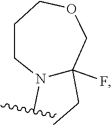 [SMILES: *N1CCCOCC1(F)CC] |
| 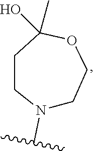 [SMILES: *N1CCOC(C)(O)CC1] | 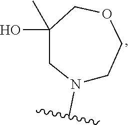 [SMILES: *N1CCOCC(C)(O)C1] |
| 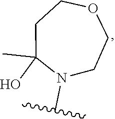 [SMILES: *N1CCOCCC1(C)O] | 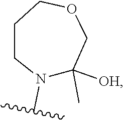 [SMILES: *N1CCCOCC1(C)O] |
| 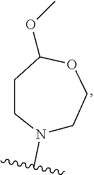 [SMILES: *N1CCOC(OC)CC1] | 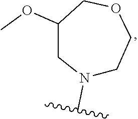 [SMILES: *N1CCOCC(OC)C1] |
| 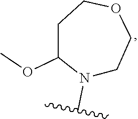 [SMILES: *N1CCOCCC1OC] | 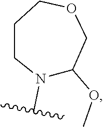 [SMILES: *N1CCCOCC1OC] |
| 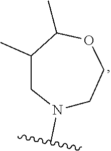 [SMILES: *N1CCOC(C)C(C)C1] | 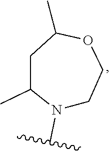 [SMILES: *N1CCOC(C)CC1C] |
| 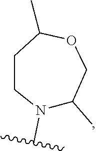 | 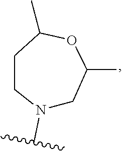 |
| 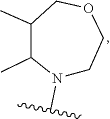 [SMILES: *N1CCOCC(C)C1C] | 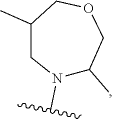 |
| 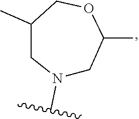 | 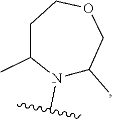 |
| 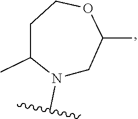 | 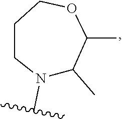 |
| 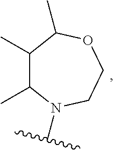 [SMILES: *N1CCOC(C)C(C)C1C] | 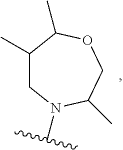 |
| 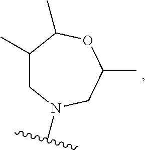 | 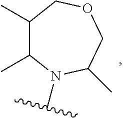 |
| 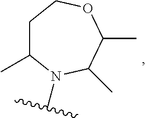 | 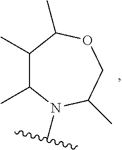 |
| 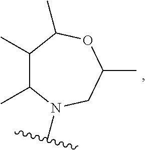 | 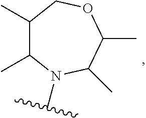 |
| 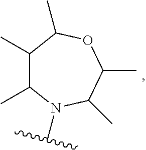 | 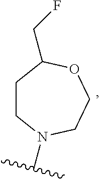 [SMILES: *N1CCOC(CF)CC1] |
| 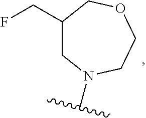 [SMILES: *N1CCOCC(CF)C1] | 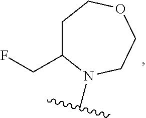 [SMILES: *N1CCOCCC1CF] |
| 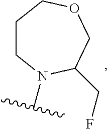 [SMILES: *N1CCCOCC1CF] | 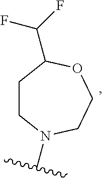 [SMILES: *N1CCOC(C(F)F)CC1] |
| 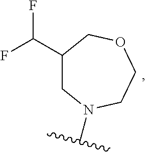 [SMILES: *N1CCOCC(C(F)F)C1] | 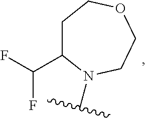 [SMILES: *N1CCOCCC1C(F)F] |
| 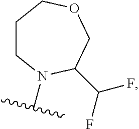 [SMILES: *N1CCCOCC1C(F)F] | 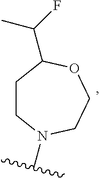 [SMILES: *N1CCOC(C(C)F)CC1] |
| 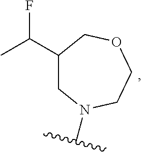 [SMILES: *N1CCOCC(C(C)F)C1] | 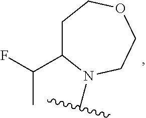 [SMILES: *N1CCOCCC1C(C)F] |
| 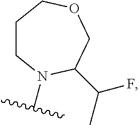 [SMILES: *N1CCCOCC1C(C)F] | 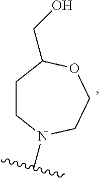 [SMILES: *N1CCOC(CO)CC1] |
| 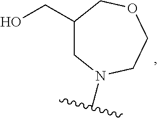 [SMILES: *N1CCOCC(CO)C1] | 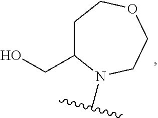 [SMILES: *N1CCOCCC1CO] |
| 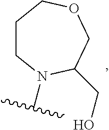 [SMILES: *N1CCCOCC1CO] | 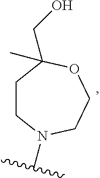 [SMILES: *N1CCOC(C)(CO)CC1] |
| 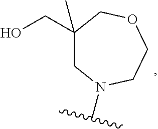 [SMILES: *N1CCOCC(C)(CO)C1] | 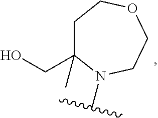 [SMILES: *N1CCOCCC1(C)CO] |
| 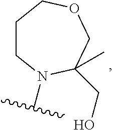 [SMILES: *N1CCCOCC1(C)CO] | 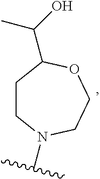 [SMILES: *N1CCOC(C(C)O)CC1] |
| 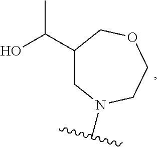 [SMILES: *N1CCOCC(C(C)O)C1] | 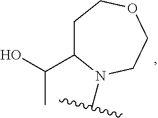 [SMILES: *N1CCOCCC1C(C)O] |
| 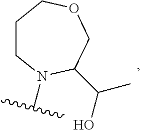 [SMILES: *N1CCCOCC1C(C)O] | 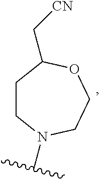 [SMILES: *N1CCOC(CC#N)CC1] |
| 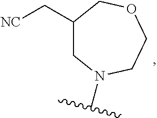 [SMILES: *N1CCOCC(CC#N)C1] | 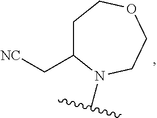 [SMILES: *N1CCOCCC1CC#N] |
| 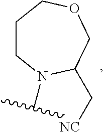 [SMILES: *N1CCCOCC1CC#N] | 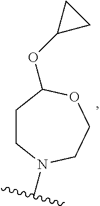 [SMILES: *N1CCOC(OC2CC2)CC1] |
| 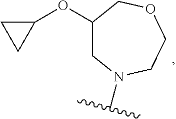 [SMILES: *N1CCOCC(OC2CC2)C1] | 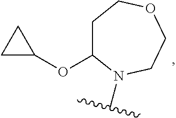 [SMILES: *N1CCOCCC1OC1CC1] |
| 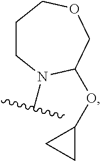 [SMILES: *N1CCCOCC1OC1CC1] | 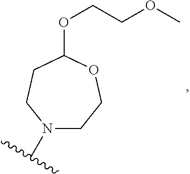 [SMILES: *N1CCOC(OCCOC)CC1] |
| 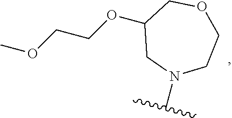 [SMILES: *N1CCOCC(OCCOC)C1] | |
| 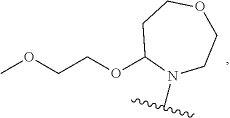 [SMILES: *N1CCOCCC1OCCOC] | |
| 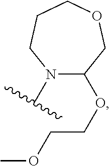 [SMILES: *N1CCCOCC1OCCOC] | 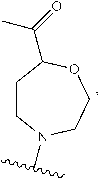 [SMILES: *N1CCOC(C(C)=O)CC1] |
| 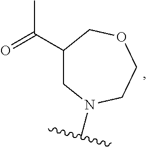 [SMILES: *N1CCOCC(C(C)=O)C1] | 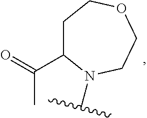 [SMILES: *N1CCOCCC1C(C)=O] |
| 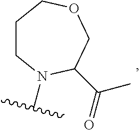 [SMILES: *N1CCCOCC1C(C)=O] | 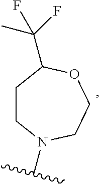 [SMILES: *N1CCOC(C(C)(F)F)CC1] |
| 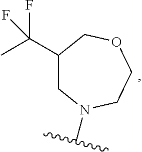 [SMILES: *N1CCOCC(C(C)(F)F)C1] | 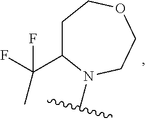 [SMILES: *N1CCOCCC1C(C)(F)F] |
| 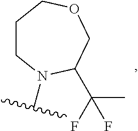 [SMILES: *N1CCCOCC1C(C)(F)F] | 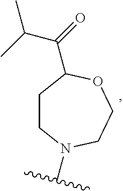 [SMILES: *N1CCOC(C(=O)C(C)C)CC1] |
| 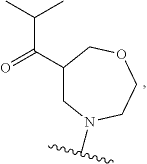 [SMILES: *N1CCOCC(C(=O)C(C)C)C1] | 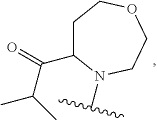 [SMILES: *N1CCOCCC1C(=O)C(C)C] |
| 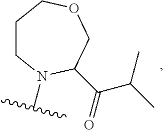 [SMILES: *N1CCCOCC1C(=O)C(C)C] | 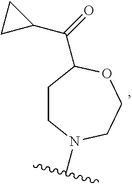 [SMILES: *N1CCOC(C(=O)C2CC2)CC1] |
| 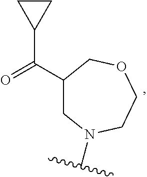 [SMILES: *N1CCOCC(C(=O)C2CC2)C1] | 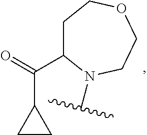 [SMILES: *N1CCOCCC1C(=O)C1CC1] |
| 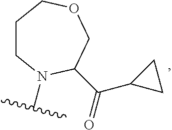 [SMILES: *N1CCCOCC1C(=O)C1CC1] | 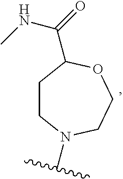 [SMILES: *N1CCOC(C(=O)NC)CC1] |
| 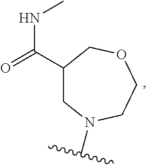 [SMILES: *N1CCOCC(C(=O)NC)C1] | 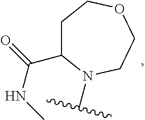 [SMILES: *N1CCOCCC1C(=O)NC] |
| 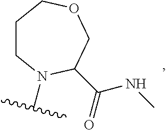 [SMILES: *N1CCCOCC1C(=O)NC] | 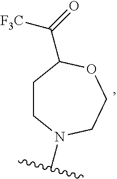 [SMILES: *N1CCOC(C(=O)C(F)(F)F)CC1] |
| 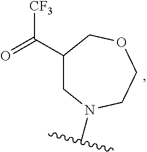 [SMILES: *N1CCOCC(C(=O)C(F)(F)F)C1] | 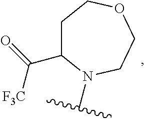 [SMILES: *N1CCOCCC1C(=O)C(F)(F)F] |
| 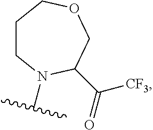 [SMILES: *N1CCCOCC1C(=O)C(F)(F)F] | 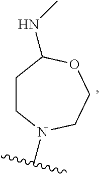 [SMILES: *N1CCOC(NC)CC1] |
| 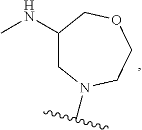 [SMILES: *N1CCOCC(NC)C1] | 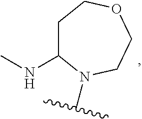 [SMILES: *N1CCOCCC1NC] |
| 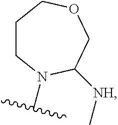 [SMILES: *N1CCCOCC1NC] | 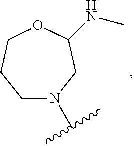 [SMILES: *N1CCCOC(NC)C1] |
| 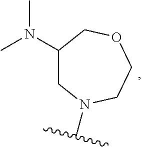 [SMILES: *N1CCOCC(N(C)C)C1] | |
| 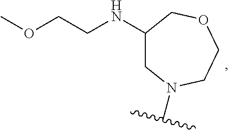 [SMILES: *N1CCOCC(NCCOC)C1] | |
| 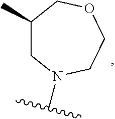 [SMILES: *N1CCOC[C@H](C)C1] | 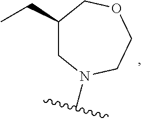 [SMILES: *N1CCOC[C@H](CC)C1] |
| 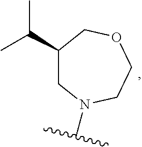 [SMILES: *N1CCOC[C@H](C(C)C)C1] | 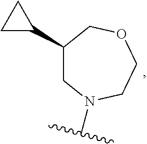 [SMILES: *N1CCOC[C@H](C2CC2)C1] |
| 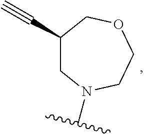 [SMILES: *N1CCOC[C@H](C#C)C1] | 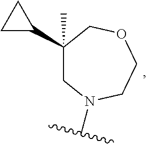 [SMILES: *N1CCOC[C@](C)(C2CC2)C1] |
| 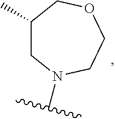 [SMILES: *N1CCOC[C@@H](C)C1] | 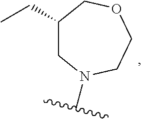 [SMILES: *N1CCOC[C@@H](CC)C1] |
| 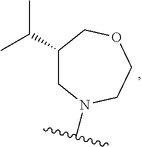 [SMILES: *N1CCOC[C@@H](C(C)C)C1] | 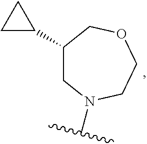 [SMILES: *N1CCOC[C@@H](C2CC2)C1] |
| 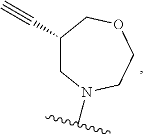 [SMILES: *N1CCOC[C@@H](C#C)C1] | 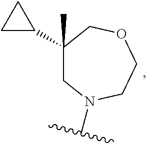 [SMILES: *N1CCOC[C@@](C)(C2CC2)C1] |
| 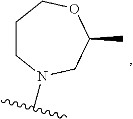 [SMILES: *N1CCCO[C@@H](C)C1] | 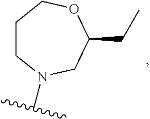 [SMILES: *N1CCCO[C@@H](CC)C1] |
| 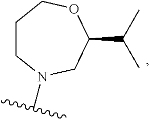 [SMILES: *N1CCCO[C@@H](C(C)C)C1] | 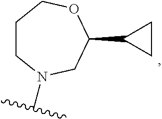 [SMILES: *N1CCCO[C@@H](C2CC2)C1] |
| 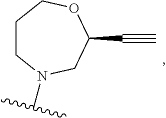 [SMILES: *N1CCCO[C@@H](C#C)C1] | 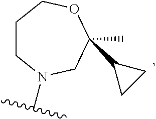 [SMILES: *N1CCCO[C@@](C)(C2CC2)C1] |
| 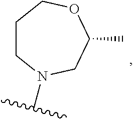 [SMILES: *N1CCCO[C@H](C)C1] | 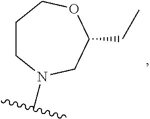 [SMILES: *N1CCCO[C@H](CC)C1] |
| 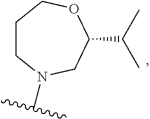 [SMILES: *N1CCCO[C@H](C(C)C)C1] | 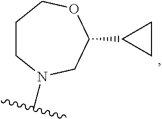 [SMILES: *N1CCCO[C@H](C2CC2)C1] |
| 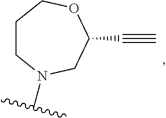 [SMILES: *N1CCCO[C@H](C#C)C1] | 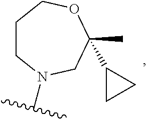 [SMILES: *N1CCCO[C@](C)(C2CC2)C1] |
| 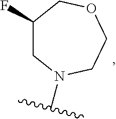 [SMILES: *N1CCOC[C@H](F)C1] | 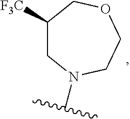 [SMILES: *N1CCOC[C@H](C(F)(F)F)C1] |
| 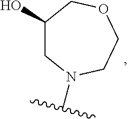 [SMILES: *N1CCOC[C@H](O)C1] | 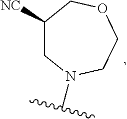 [SMILES: *N1CCOC[C@H](C#N)C1] |
| 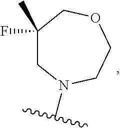 [SMILES: *N1CCOC[C@@](C)(F)C1] | 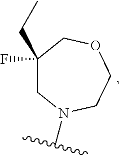 [SMILES: *N1CCOC[C@](F)(CC)C1] |
| 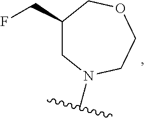 [SMILES: *N1CCOC[C@H](CF)C1] | 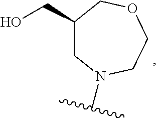 [SMILES: *N1CCOC[C@H](CO)C1] |
| 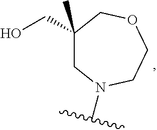 [SMILES: *N1CCOC[C@@](C)(CO)C1] | 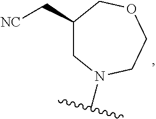 [SMILES: *N1CCOC[C@H](CC#N)C1] |
| 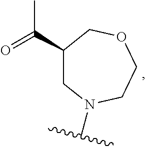 [SMILES: *N1CCOC[C@H](C(C)=O)C1] | 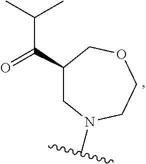 [SMILES: *N1CCOC[C@H](C(=O)C(C)C)C1] |
| 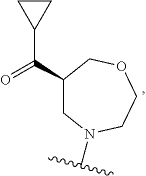 [SMILES: *N1CCOC[C@H](C(=O)C2CC2)C1] | 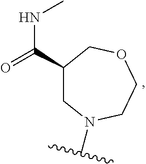 [SMILES: *N1CCOC[C@H](C(=O)NC)C1] |
| 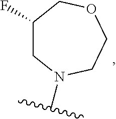 [SMILES: *N1CCOC[C@@H](F)C1] | 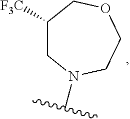 [SMILES: *N1CCOC[C@@H](C(F)(F)F)C1] |
| 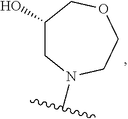 [SMILES: *N1CCOC[C@@H](O)C1] | 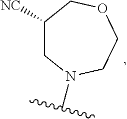 [SMILES: *N1CCOC[C@@H](C#N)C1] |
| 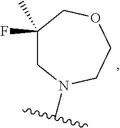 [SMILES: *N1CCOC[C@](C)(F)C1] | 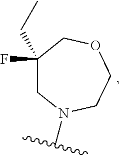 [SMILES: *N1CCOC[C@@](F)(CC)C1] |
| 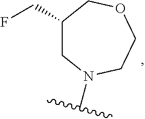 [SMILES: *N1CCOC[C@@H](CF)C1] | 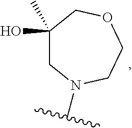 [SMILES: *N1CCOC[C@](C)(O)C1] |
| 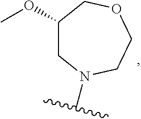 [SMILES: *N1CCOC[C@@H](OC)C1] | 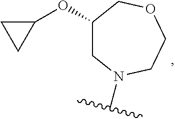 [SMILES: *N1CCOC[C@@H](OC2CC2)C1] |
| 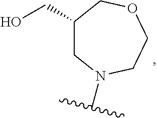 [SMILES: *N1CCOC[C@@H](CO)C1] | 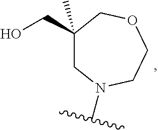 [SMILES: *N1CCOC[C@](C)(CO)C1] |
| 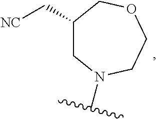 [SMILES: *N1CCOC[C@@H](CC#N)C1] | |
| 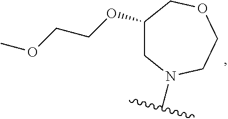 [SMILES: *N1CCOC[C@@H](OCCOC)C1] | |
| 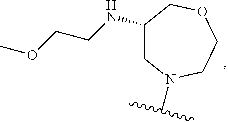 [SMILES: *N1CCOC[C@@H](NCCOC)C1] | |
| 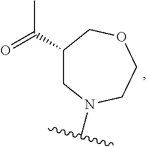 [SMILES: *N1CCOC[C@@H](C(C)=O)C1] | 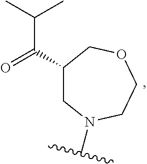 [SMILES: *N1CCOC[C@@H](C(=O)C(C)C)C1] |
| 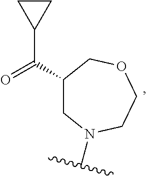 [SMILES: *N1CCOC[C@@H](C(=O)C2CC2)C1] | 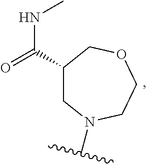 [SMILES: *N1CCOC[C@@H](C(=O)NC)C1] |
| 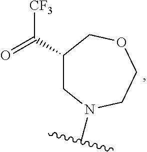 [SMILES: *N1CCOC[C@@H](C(=O)C(F)(F)F)C1] | 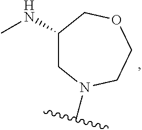 [SMILES: *N1CCOC[C@@H](NC)C1] |
| 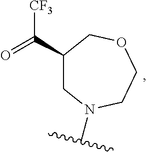 [SMILES: *N1CCOC[C@H](C(=O)C(F)(F)F)C1] | 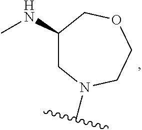 [SMILES: *N1CCOC[C@H](NC)C1] |
| 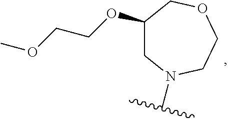 [SMILES: *N1CCOC[C@H](OCCOC)C1] | |
| 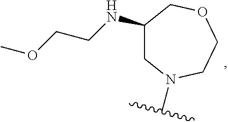 [SMILES: *N1CCOC[C@H](NCCOC)C1] | |
| 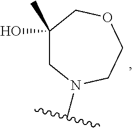 [SMILES: *N1CCOC[C@@](C)(O)C1] | 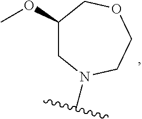 [SMILES: *N1CCOC[C@H](OC)C1] |
| 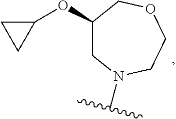 [SMILES: *N1CCOC[C@H](OC2CC2)C1] | 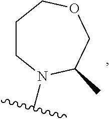 [SMILES: *N1CCCOC[C@H]1C] |
| 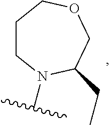 [SMILES: *N1CCCOC[C@H]1CC] | 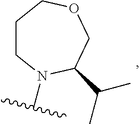 [SMILES: *N1CCCOC[C@H]1C(C)C] |
| 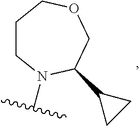 [SMILES: *N1CCCOC[C@H]1C1CC1] | 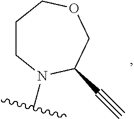 [SMILES: *N1CCCOC[C@H]1C#C] |
| 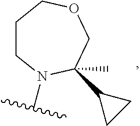 [SMILES: *N1CCCOC[C@@]1(C)C1CC1] | 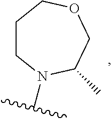 [SMILES: *N1CCCOC[C@@H]1C] |
| 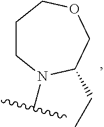 [SMILES: *N1CCCOC[C@@H]1CC] | 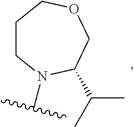 [SMILES: *N1CCCOC[C@@H]1C(C)C] |
| 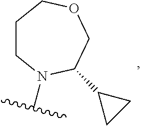 [SMILES: *N1CCCOC[C@@H]1C1CC1] | 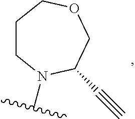 [SMILES: *N1CCCOC[C@@H]1C#C] |
| 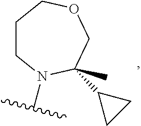 [SMILES: *N1CCCOC[C@]1(C)C1CC1] | 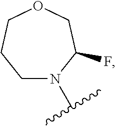 [SMILES: *N1CCCOC[C@H]1F] |
| 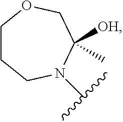 [SMILES: *N1CCCOC[C@@]1(C)O] | 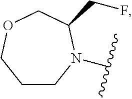 [SMILES: *N1CCCOC[C@H]1CF] |
| 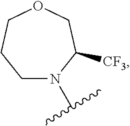 [SMILES: *N1CCCOC[C@H]1C(F)(F)F] | 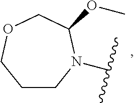 [SMILES: *N1CCCOC[C@H]1OC] |
| 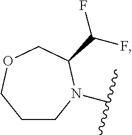 [SMILES: *N1CCCOC[C@H]1C(F)F] | 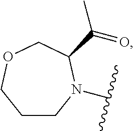 [SMILES: *N1CCCOC[C@H]1C(C)=O] |
| 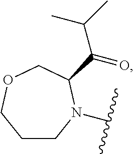 [SMILES: *N1CCCOC[C@H]1C(=O)C(C)C] | 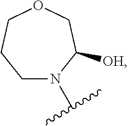 [SMILES: *N1CCCOC[C@H]1O] |
| 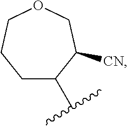 [SMILES: *C1CCCOC[C@H]1C#N] | 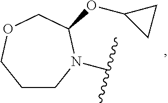 [SMILES: *N1CCCOC[C@H]1OC1CC1] |
| 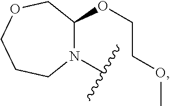 [SMILES: *N1CCCOC[C@H]1OCCOC] | 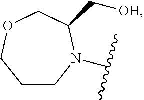 [SMILES: *N1CCCOC[C@H]1CO] |
| 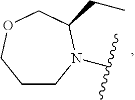 [SMILES: *N1CCCOC[C@H]1CC] | 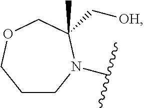 [SMILES: *N1CCCOC[C@]1(C)CO] |
| 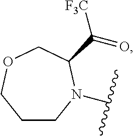 [SMILES: *N1CCCOC[C@H]1C(=O)C(F)(F)F] | 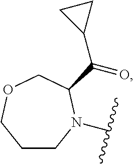 [SMILES: *N1CCCOC[C@H]1C(=O)C1CC1] |
| 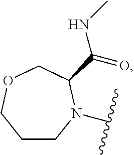 [SMILES: *N1CCCOC[C@H]1C(=O)NC] | 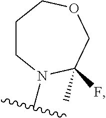 [SMILES: *N1CCCOC[C@@]1(C)F] |
| 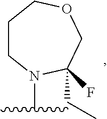 [SMILES: *N1CCCOC[C@]1(F)CC] | 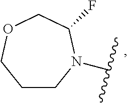 [SMILES: *N1CCCOC[C@@H]1F] |
| 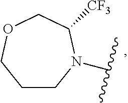 [SMILES: *N1CCCOC[C@@H]1C(F)(F)F] | 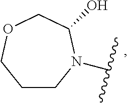 [SMILES: *N1CCCOC[C@@H]1O] |
| 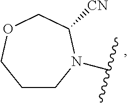 [SMILES: *N1CCCOC[C@@H]1C#N] | 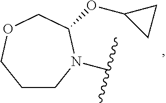 [SMILES: *N1CCCOC[C@@H]1OC1CC1] |
| 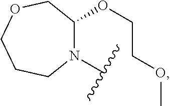 [SMILES: *N1CCCOC[C@@H]1OCCOC] | 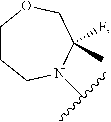 [SMILES: *N1CCCOC[C@]1(C)F] |
| 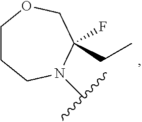 [SMILES: *N1CCCOC[C@@]1(F)CC] | 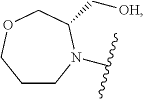 [SMILES: *N1CCCOC[C@@H]1CO] |
| 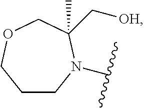 [SMILES: *N1CCCOC[C@@]1(C)CO] | 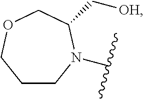 [SMILES: *N1CCCOC[C@@H]1CO] |
| 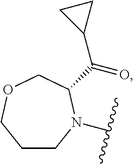 [SMILES: *N1CCCOC[C@@H]1C(=O)C1CC1] | 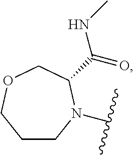 [SMILES: *N1CCCOC[C@@H]1C(=O)NC] |
| 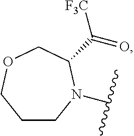 [SMILES: *N1CCCOC[C@@H]1C(=O)C(F)(F)F] | 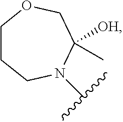 [SMILES: *N1CCCOC[C@]1(C)O] |
| 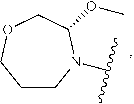 [SMILES: *N1CCCOC[C@@H]1OC] | 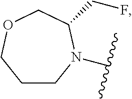 [SMILES: *N1CCCOC[C@@H]1CF] |
| 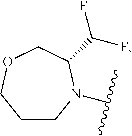 [SMILES: *N1CCCOC[C@@H]1C(F)F] | 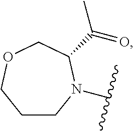 [SMILES: *N1CCCOC[C@@H]1C(C)=O] |
| 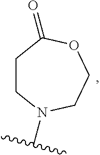 [SMILES: *N1CCOC(=O)CC1] | 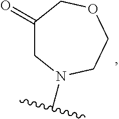 [SMILES: *N1CCOCC(=O)C1] |
| 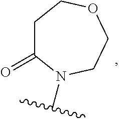 [SMILES: *N1CCOCCC1=O] | 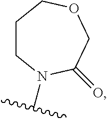 [SMILES: *N1CCCOCC1=O] |
| 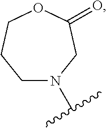 [SMILES: *N1CCCOC(=O)C1] | 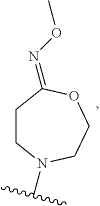 [SMILES: *N1CCO/C(=N\OC)CC1] |
| 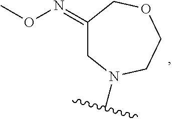 [SMILES: *N1CCOC/C(=N/OC)C1] | 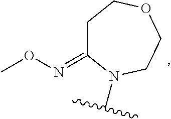 [SMILES: *N1CCOCC/C1=N\OC] |
| 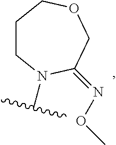 [SMILES: *N1CCCOC/C1=N/OC] | 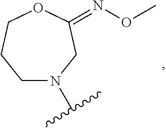 [SMILES: *N1CCCO/C(=N/OC)C1] |
| 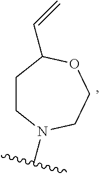 [SMILES: *N1CCOC(C=C)CC1] | 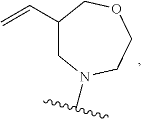 [SMILES: *N1CCOCC(C=C)C1] |
| 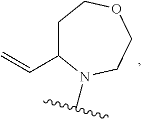 [SMILES: *N1CCOCCC1C=C] | 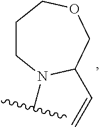 [SMILES: *N1CCCOCC1C=C] |
| 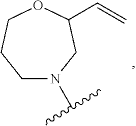 [SMILES: *N1CCCOC(C=C)C1] | 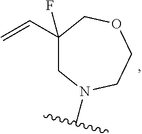 [SMILES: *N1CCOCC(F)(C=C)C1] |
| 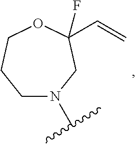 [SMILES: *N1CCCOC(F)(C=C)C1] | 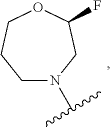 [SMILES: *N1CCCO[C@@H](F)C1] |
| 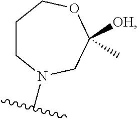 [SMILES: *N1CCCO[C@@](C)(O)C1] | 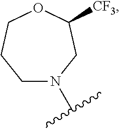 [SMILES: *N1CCCO[C@@H](C(F)(F)F)C1] |
| 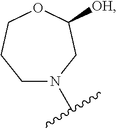 [SMILES: *N1CCCO[C@@H](O)C1] | 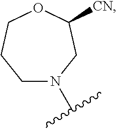 [SMILES: *N1CCCO[C@@H](C#N)C1] |
| 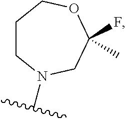 [SMILES: *N1CCCO[C@@](C)(F)C1] | 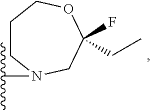 [SMILES: *N1CCCO[C@](F)(CC)C1] |
| 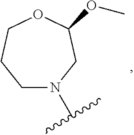 [SMILES: *N1CCCO[C@@H](OC)C1] | 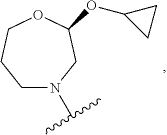 [SMILES: *N1CCCO[C@@H](OC2CC2)C1] |
| 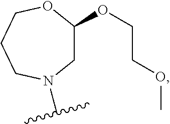 [SMILES: *N1CCCO[C@@H](OCCOC)C1] | 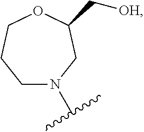 [SMILES: *N1CCCO[C@@H](CO)C1] |
| 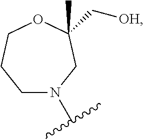 [SMILES: *N1CCCO[C@](C)(CO)C1] | 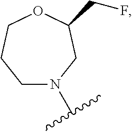 [SMILES: *N1CCCO[C@@H](CF)C1] |
| 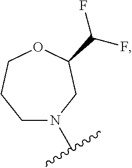 [SMILES: *N1CCCO[C@@H](C(F)F)C1] | 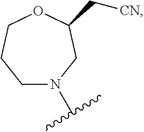 [SMILES: *N1CCCO[C@@H](CC#N)C1] |
| 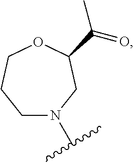 [SMILES: *N1CCCO[C@@H](C(C)=O)C1] | 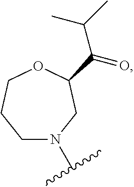 [SMILES: *N1CCCO[C@@H](C(=O)C(C)C)C1] |
| 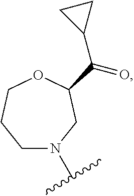 [SMILES: *N1CCCO[C@@H](C(=O)C2CC2)C1] | 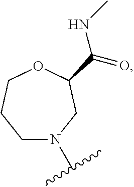 [SMILES: *N1CCCO[C@@H](C(=O)NC)C1] |
| 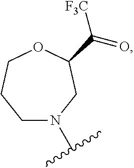 [SMILES: *N1CCCO[C@@H](C(=O)C(F)(F)F)C1] | 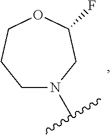 [SMILES: *N1CCCO[C@H](F)C1] |
| 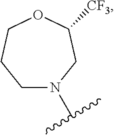 [SMILES: *N1CCCO[C@H](C(F)(F)F)C1] | 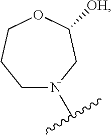 [SMILES: *N1CCCO[C@H](O)C1] |
| 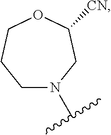 [SMILES: *N1CCCO[C@H](C#N)C1] | 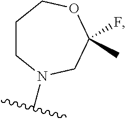 [SMILES: *N1CCCO[C@](C)(F)C1] |
| 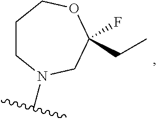 [SMILES: *N1CCCO[C@@](F)(CC)C1] | 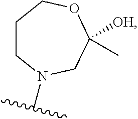 [SMILES: *N1CCCO[C@](C)(O)C1] |
| 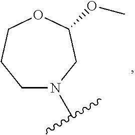 [SMILES: *N1CCCO[C@H](OC)C1] | 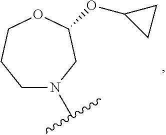 [SMILES: *N1CCCO[C@H](OC2CC2)C1] |
| 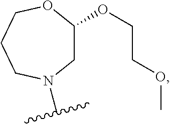 [SMILES: *N1CCCO[C@H](OCCOC)C1] | 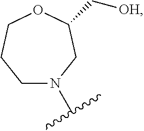 [SMILES: *N1CCCO[C@H](CO)C1] |
| 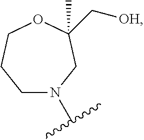 [SMILES: *N1CCCO[C@@](C)(CO)C1] | 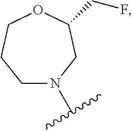 [SMILES: *N1CCCO[C@H](CF)C1] |
| 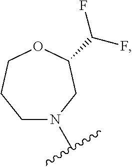 [SMILES: *N1CCCO[C@H](C(F)F)C1] | 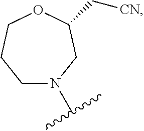 [SMILES: *N1CCCO[C@H](CC#N)C1] |
| 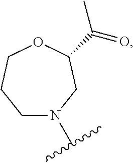 [SMILES: *N1CCCO[C@H](C(C)=O)C1] | 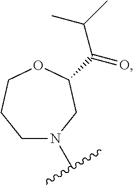 [SMILES: *N1CCCO[C@H](C(=O)C(C)C)C1] |
| 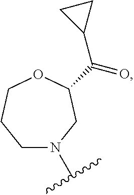 [SMILES: *N1CCCO[C@H](C(=O)C2CC2)C1] | 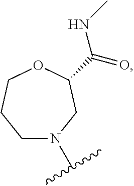 [SMILES: *N1CCCO[C@H](C(=O)NC)C1] |
| 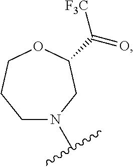 [SMILES: *N1CCCO[C@H](C(=O)C(F)(F)F)C1] | 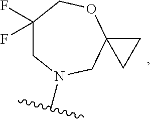 [SMILES: *N1CC(F)(F)COC2(CC2)C1] |
| 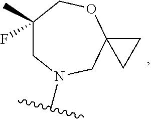 [SMILES: *N1CC2(CC2)OC[C@@](C)(F)C1] | 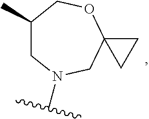 [SMILES: *N1C[C@@H](C)COC2(CC2)C1] |
| 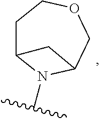 [SMILES: *N1C2CCOCC1C2] | 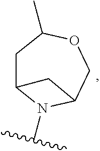 [SMILES: *N1C2COC(C)CC1C2] |
| 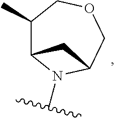 [SMILES: *N1[C@H]2COC[C@H](C)[C@@H]1C2] | 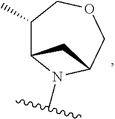 [SMILES: *N1[C@H]2COC[C@@H](C)[C@@H]1C2] |
| 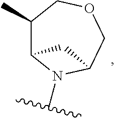 [SMILES: *N1[C@@H]2COC[C@H](C)[C@H]1C2] | 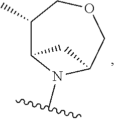 [SMILES: *N1[C@@H]2COC[C@@H](C)[C@H]1C2] |
| 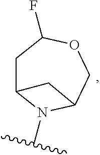 [SMILES: *N1C2COC(F)CC1C2] | 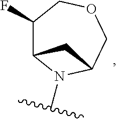 [SMILES: *N1[C@H]2COC[C@H](F)[C@@H]1C2] |
| 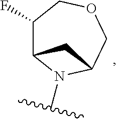 [SMILES: *N1[C@H]2COC[C@@H](F)[C@@H]1C2] | 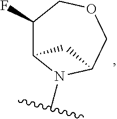 [SMILES: *N1[C@@H]2COC[C@H](F)[C@H]1C2] |
| 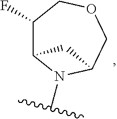 [SMILES: *N1[C@@H]2COC[C@@H](F)[C@H]1C2] | 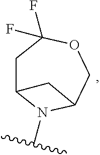 [SMILES: *N1C2COC(F)(F)CC1C2] |
| 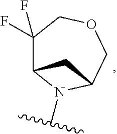 [SMILES: *N1[C@H]2COCC(F)(F)[C@@H]1C2] | 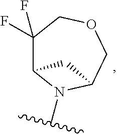 [SMILES: *N1[C@@H]2COCC(F)(F)[C@H]1C2] |
| 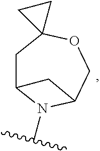 [SMILES: *N1C2COC3(CC3)CC1C2] | 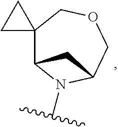 [SMILES: *N1[C@H]2COCC3(CC3)[C@@H]1C2] |
| 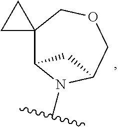 [SMILES: *N1[C@@H]2COCC3(CC3)[C@H]1C2] | 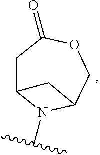 [SMILES: *N1C2COC(=O)CC1C2] |
| 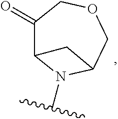 [SMILES: *N1C2COCC(=O)C1C2] | 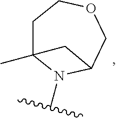 [SMILES: *N1C2COCCC1(C)C2] |
| 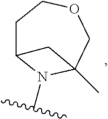 [SMILES: *N1C2CCOCC1(C)C2] | 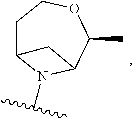 [SMILES: *N1C2CCO[C@@H](C)C1C2] |
| 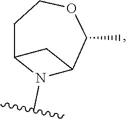 [SMILES: *N1C2CCO[C@H](C)C1C2] | 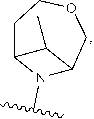 [SMILES: *N1C2CCOCC1C2C] |
| 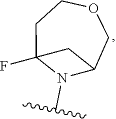 [SMILES: *N1C2COCCC1(F)C2] | 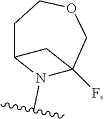 [SMILES: *N1C2CCOCC1(F)C2] |
| 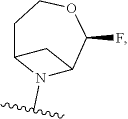 [SMILES: *N1C2CCO[C@@H](F)C1C2] | 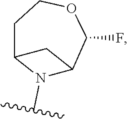 [SMILES: *N1C2CCO[C@H](F)C1C2] |
| 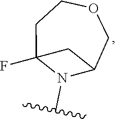 [SMILES: *N1C2COCCC1(F)C2] | 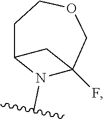 [SMILES: *N1C2CCOCC1(F)C2] |
| 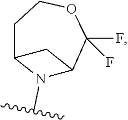 [SMILES: *N1C2CCOC(F)(F)C1C2] | 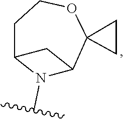 [SMILES: *N1C2CCOC3(CC3)C1C2] |
| 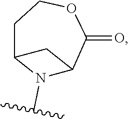 [SMILES: *N1C2CCOC(=O)C1C2] | 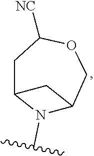 [SMILES: *N1C2COC(C#N)CC1C2] |
| 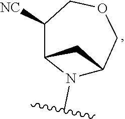 [SMILES: *N1[C@H]2COC[C@H](C#N)[C@@H]1C2] | 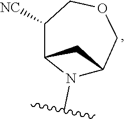 [SMILES: *N1[C@H]2COC[C@@H](C#N)[C@@H]1C2] |
| 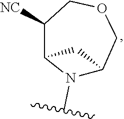 [SMILES: *N1[C@@H]2COC[C@H](C#N)[C@H]1C2] | 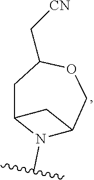 [SMILES: *N1C2COC(CC#N)CC1C2] |
| 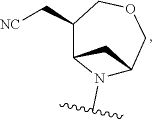 [SMILES: *N1[C@H]2COC[C@H](CC#N)[C@@H]1C2] | 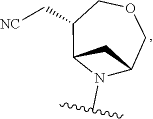 [SMILES: *N1[C@H]2COC[C@@H](CC#N)[C@@H]1C2] |
| 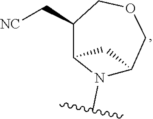 [SMILES: *N1[C@@H]2COC[C@H](CC#N)[C@H]1C2] | 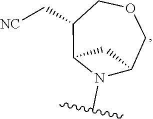 [SMILES: *N1[C@@H]2COC[C@@H](CC#N)[C@H]1C2] |
| 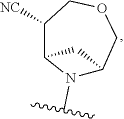 [SMILES: *N1[C@@H]2COC[C@@H](C#N)[C@H]1C2] | 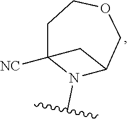 [SMILES: *N1C2COCCC1(C#N)C2] |
| 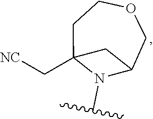 [SMILES: *N1C2COCCC1(CC#N)C2] | 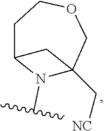 [SMILES: *N1C2CCOCC1(CC#N)C2] |
| 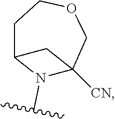 [SMILES: *N1C2CCOCC1(C#N)C2] | 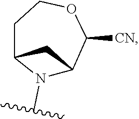 [SMILES: *N1[C@@H]2CCO[C@@H](C#N)[C@H]1C2] |
| 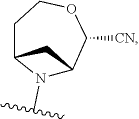 [SMILES: *N1[C@@H]2CCO[C@H](C#N)[C@H]1C2] | 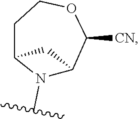 [SMILES: *N1[C@H]2CCO[C@@H](C#N)[C@@H]1C2] |
| 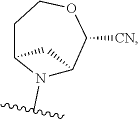 [SMILES: *N1[C@H]2CCO[C@H](C#N)[C@@H]1C2] | 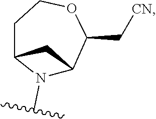 [SMILES: *N1[C@@H]2CCO[C@@H](CC#N)[C@H]1C2] |
| 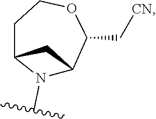 [SMILES: *N1[C@@H]2CCO[C@H](CC#N)[C@H]1C2] | 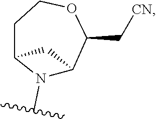 [SMILES: *N1[C@H]2CCO[C@@H](CC#N)[C@@H]1C2] |
| 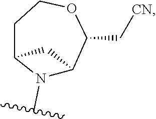 [SMILES: *N1[C@H]2CCO[C@H](CC#N)[C@@H]1C2] | 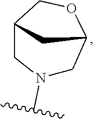 [SMILES: *N1C[C@@H]2CO[C@@H](C2)C1] |
| 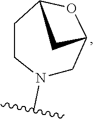 [SMILES: *N1CC[C@H]2C[C@@H](C1)O2] | 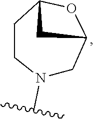 [SMILES: *N1CC[C@H]2C[C@@H](C1)O2] |
| 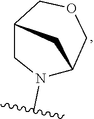 [SMILES: *N1C[C@@H]2COC[C@H]1C2] | 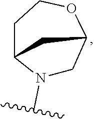 [SMILES: *N1C[C@@H]2C[C@H]1CCO2] |
| 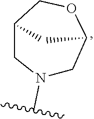 [SMILES: *N1C[C@H]2CO[C@H](C2)C1] | 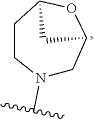 [SMILES: *N1CC[C@@H]2C[C@H](C1)O2] |
| 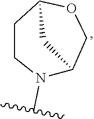 [SMILES: *N1CC[C@@H]2C[C@H]1CO2] | 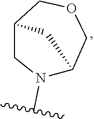 [SMILES: *N1C[C@H]2COC[C@@H]1C2] |
| 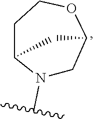 [SMILES: *N1C[C@H]2C[C@@H]1CCO2] | 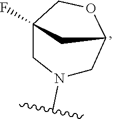 |
| 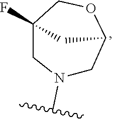 | 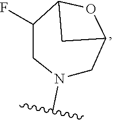 [SMILES: *N1CC2CC(O2)C(F)C1] |
| 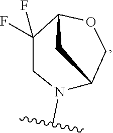 [SMILES: *N1CC(F)(F)[C@H]2C[C@@H]1CO2] | 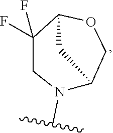 [SMILES: *N1CC(F)(F)[C@@H]2C[C@H]1CO2] |
| 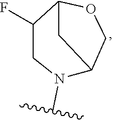 [SMILES: *N1CC(F)C2CC1CO2] | 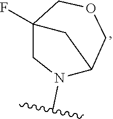 [SMILES: *N1CC2(F)COCC1C2] |
| 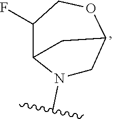 [SMILES: *N1CC2CC1C(F)CO2] | 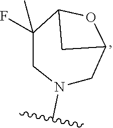 [SMILES: *N1CC2CC(O2)C(C)(F)C1] |
| 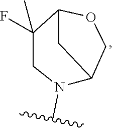 [SMILES: *N1CC(C)(F)C2CC1CO2] | 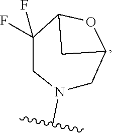 [SMILES: *N1CC2CC(O2)C(F)(F)C1] |
| 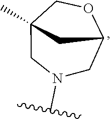 | 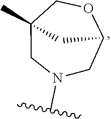 |
| 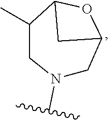 [SMILES: *N1CC2CC(O2)C(C)C1] | 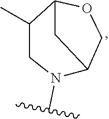 [SMILES: *N1CC(C)C2CC1CO2] |
| 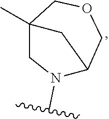 [SMILES: *N1CC2(C)COCC1C2] | 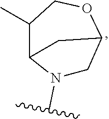 [SMILES: *N1CC2CC1C(C)CO2] |
| 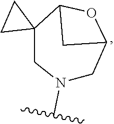 [SMILES: *N1CC2CC(O2)C2(CC2)C1] | 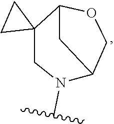 [SMILES: *N1CC2(CC2)C2CC1CO2] |
| 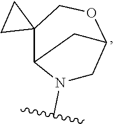 [SMILES: *N1CC2CC1C1(CC1)CO2] | 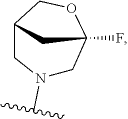 |
| 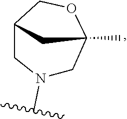 | 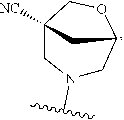 |
| 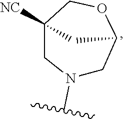 | 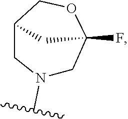 |
| 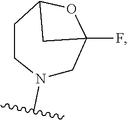 [SMILES: *N1CCC2CC(F)(C1)O2] | 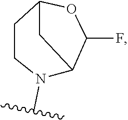 [SMILES: *N1CCC2CC1C(F)O2] |
| 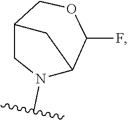 [SMILES: *N1CC2COC(F)C1C2] | 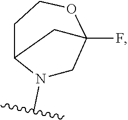 [SMILES: *N1CC2(F)CC1CCO2] |
| 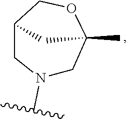 | 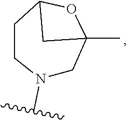 [SMILES: *N1CCC2CC(C)(C1)O2] |
| 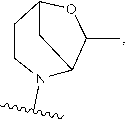 [SMILES: *N1CCC2CC1C(C)O2] | 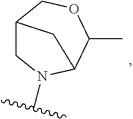 [SMILES: *N1CC2COC(C)C1C2] |
| 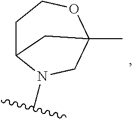 [SMILES: *N1CC2(C)CC1CCO2] | 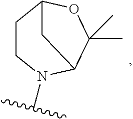 [SMILES: *N1CCC2CC1C(C)(C)O2] |
| 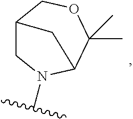 [SMILES: *N1CC2COC(C)(C)C1C2] | 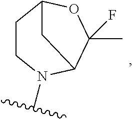 [SMILES: *N1CCC2CC1C(C)(F)O2] |
| 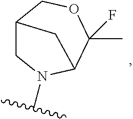 [SMILES: *N1CC2COC(C)(F)C1C2] | 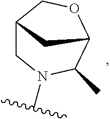 [SMILES: *N1C[C@@H]2CO[C@@H](C2)[C@H]1C] |
| 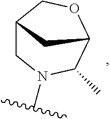 [SMILES: *N1C[C@@H]2CO[C@@H](C2)[C@@H]1C] | 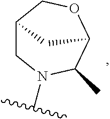 [SMILES: *N1C[C@H]2CO[C@H](C2)[C@H]1C] |
| 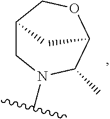 [SMILES: *N1C[C@H]2CO[C@H](C2)[C@@H]1C] | 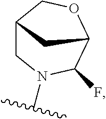 [SMILES: *N1C[C@@H]2CO[C@@H](C2)[C@H]1F] |
| 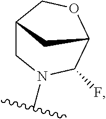 [SMILES: *N1C[C@@H]2CO[C@@H](C2)[C@@H]1F] | 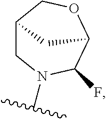 [SMILES: *N1C[C@H]2CO[C@H](C2)[C@H]1F] |
| 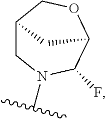 [SMILES: *N1C[C@H]2CO[C@H](C2)[C@@H]1F] | 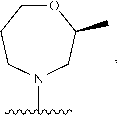 [SMILES: *N1CCCO[C@@H](C)C1] |
| 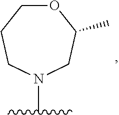 [SMILES: *N1CCCO[C@H](C)C1] | 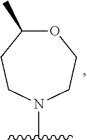 [SMILES: *N1CCO[C@H](C)CC1] |
| 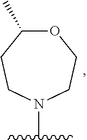 [SMILES: *N1CCO[C@@H](C)CC1] | 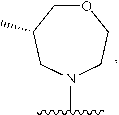 [SMILES: *N1CCOC[C@@H](C)C1] |
| 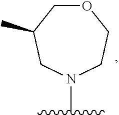 [SMILES: *N1CCOC[C@H](C)C1] | 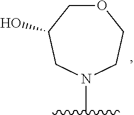 [SMILES: *N1CCOC[C@@H](O)C1] |
| 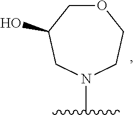 [SMILES: *N1CCOC[C@H](O)C1] | 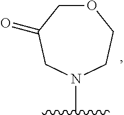 [SMILES: *N1CCOCC(=O)C1] |
| 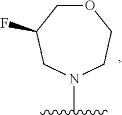 [SMILES: *N1CCOC[C@H](F)C1] | 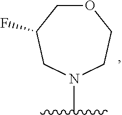 [SMILES: *N1CCOC[C@@H](F)C1] |
| 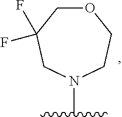 [SMILES: *N1CCOCC(F)(F)C1] | 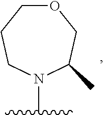 [SMILES: *N1CCCOC[C@H]1C] |
| 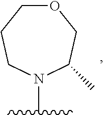 [SMILES: *N1CCCOC[C@@H]1C] | 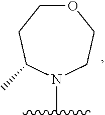 [SMILES: *N1CCOCC[C@H]1C] |
| 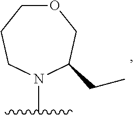 [SMILES: *N1CCCOC[C@H]1CC] | 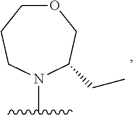 [SMILES: *N1CCCOC[C@@H]1CC] |
| 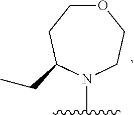 [SMILES: *N1CCOCC[C@@H]1CC] | 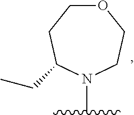 [SMILES: *N1CCOCC[C@H]1CC] |
| 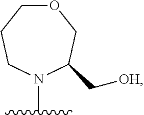 [SMILES: *N1CCCOC[C@H]1CO] | 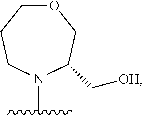 [SMILES: *N1CCCOC[C@@H]1CO] |
| 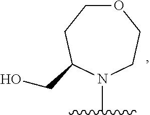 [SMILES: *N1CCOCC[C@@H]1CO] | 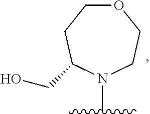 [SMILES: *N1CCOCC[C@H]1CO] |
| 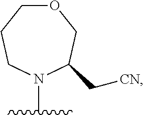 [SMILES: *N1CCCOC[C@H]1CC#N] | 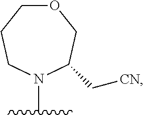 [SMILES: *N1CCCOC[C@@H]1CC#N] |
| 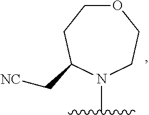 [SMILES: *N1CCOCC[C@@H]1CC#N] | 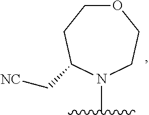 [SMILES: *N1CCOCC[C@H]1CC#N] |
| 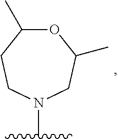 [SMILES: *N1CCC(C)OC(C)C1] | 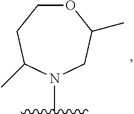 |
| 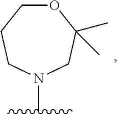 [SMILES: *N1CCCOC(C)(C)C1] | 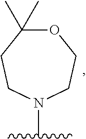 [SMILES: *N1CCOC(C)(C)CC1] |
| 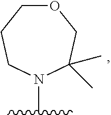 [SMILES: *N1CCCOCC1(C)C] | 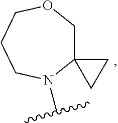 [SMILES: *N1CCCOCC12CC2] |
| 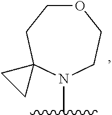 [SMILES: *N1CCOCCC12CC2] | 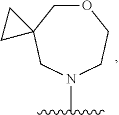 [SMILES: *N1CCOCC2(CC2)C1] |
| 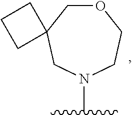 [SMILES: *N1CCOCC2(CCC2)C1] | 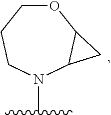 [SMILES: *N1CCCOC2CC21] |
| 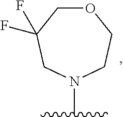 [SMILES: *N1CCOCC(F)(F)C1] | 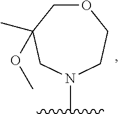 [SMILES: *N1CCOCC(C)(OC)C1] |
| 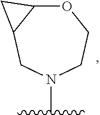 [SMILES: *N1CCOC2CC2C1] | 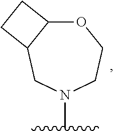 [SMILES: *N1CCOC2CCC2C1] |
| 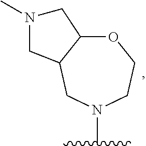 [SMILES: *N1CCOC2CN(C)CC2C1] | 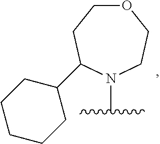 [SMILES: *N1CCOCCC1C1CCCCC1] |
| 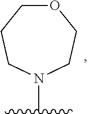 [SMILES: *N1CCCOCC1] | 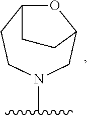 [SMILES: *N1CCC2CCC(C1)O2] |
| 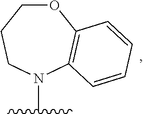 [SMILES: *N1CCCOc2ccccc21] | 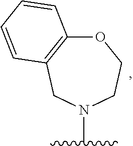 [SMILES: *N1CCOc2ccccc2C1] |
| 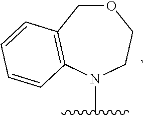 [SMILES: *N1CCOCc2ccccc21] | 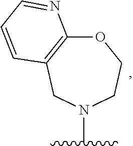 [SMILES: *N1CCOc2ncccc2C1] |
| 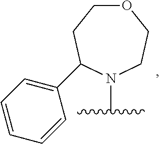 [SMILES: *N1CCOCCC1c1ccccc1] | 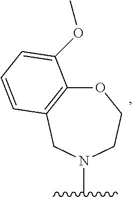 [SMILES: *N1CCOc2c(cccc2OC)C1] |
| 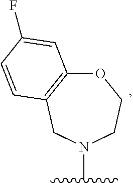 [SMILES: *N1CCOc2cc(F)ccc2C1] | 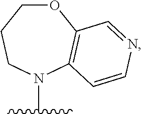 [SMILES: *N1CCCOc2cnccc21] |
| 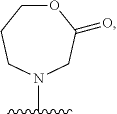 [SMILES: *N1CCCOC(=O)C1] | 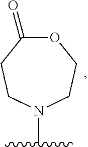 [SMILES: *N1CCOC(=O)CC1] |
| 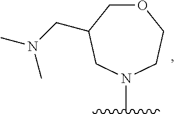 [SMILES: *N1CCOCC(CN(C)C)C1] | 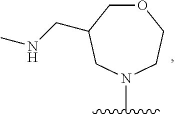 [SMILES: *N1CCOCC(CNC)C1] |
| 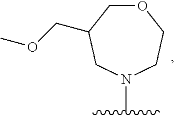 [SMILES: *N1CCOCC(COC)C1] | 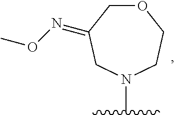 [SMILES: *N1CCOC/C(=N/OC)C1] |
| 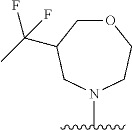 [SMILES: *N1CCOCC(C(C)(F)F)C1] | 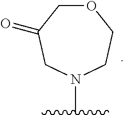 [SMILES: *N1CCOCC(=O)C1] |

[13]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [11], wherein, the moiety of

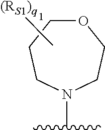

is selected from any one in the Table 2:

TABLE 2

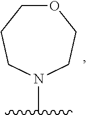 [SMILES: *N1CCCOCC1]

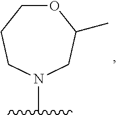 [SMILES: *N1CCCOC(C)C1]

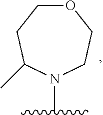 [SMILES: *N1CCOCCC1C]

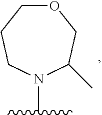 [SMILES: *N1CCCOCC1C]

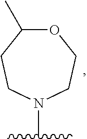 [SMILES: *N1CCOC(C)CC1]

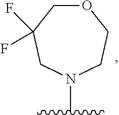 [SMILES: *N1CCOCC(F)(F)C1]

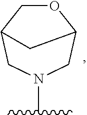 [SMILES: *N1CC2COC(C2)C1]

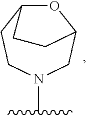 [SMILES: *N1CCC2CCC(C1)O2]

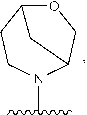 [SMILES: *N1CCC2CC1CO2]

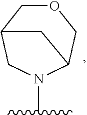 [SMILES: *N1CC2COCC1C2]

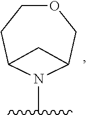 [SMILES: *N1C2CCOCC1C2]

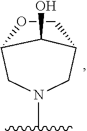 [SMILES: *N1C[C@H]2CO[C@@H](C1)[C@H]2O]

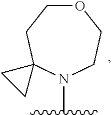 [SMILES: *N1CCOCCC12CC2]

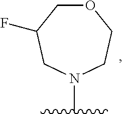 [SMILES: *N1CCOCC(F)C1]

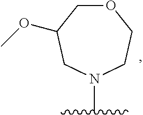 [SMILES: *N1CCOCC(OC)C1]

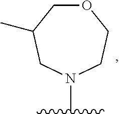 [SMILES: *N1CCOCC(C)C1]

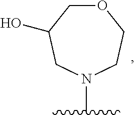 [SMILES: *N1CCOCC(O)C1]

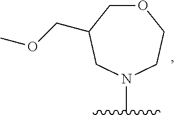 [SMILES: *N1CCOCC(COC)C1]

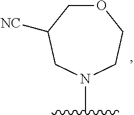 [SMILES: *N1CCOCC(C#N)C1]

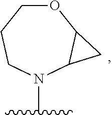 [SMILES: *N1CCCOC2CC21]

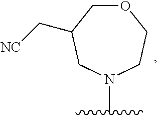 [SMILES: *N1CCOCC(CC#N)C1]

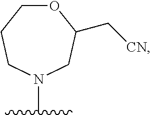 [SMILES: *N1CCCOC(CC#N)C1]

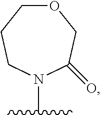 [SMILES: *N1CCCOCC1=O]

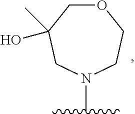 [SMILES: *N1CCOCC(C)(O)C1]

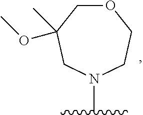 [SMILES: *N1CCOCC(C)(OC)C1]

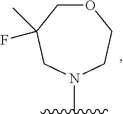 [SMILES: *N1CCOCC(C)(F)C1]

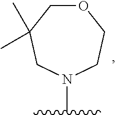 [SMILES: *N1CCOCC(C)(C)C1]

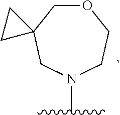 [SMILES: *N1CCOCC2(CC2)C1]

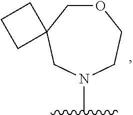 [SMILES: *N1CCOCC2(CCC2)C1]

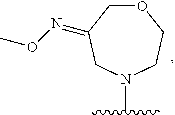 [SMILES: *N1CCOC/C(=N/OC)C1]

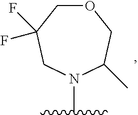 [SMILES: *N1CC(F)(F)COCC1C]

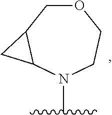 [SMILES: *N1CCOCC2CC21]

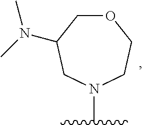 [SMILES: *N1CCOCC(N(C)C)C1]

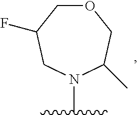 [SMILES: *N1CC(F)COCC1C]

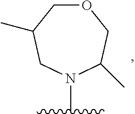

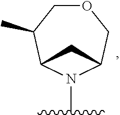 [SMILES: *N1[C@H]2COC[C@H](C)[C@@H]1C2]

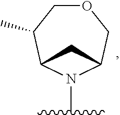 [SMILES: *N1[C@H]2COC[C@@H](C)[C@@H]1C2]

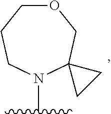 [SMILES: *N1CCCOCC12CC2]

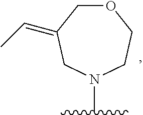 [SMILES: *N1CCOC/C(=C/C)C1]

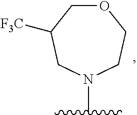 [SMILES: *N1CCOCC(C(F)(F)F)C1]

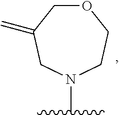 [SMILES: *N1CCOCC(=C)C1]

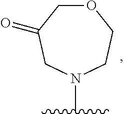 [SMILES: *N1CCOCC(=O)C1]

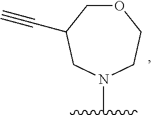 [SMILES: *N1CCOCC(C#C)C1]

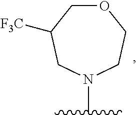 [SMILES: *N1CCOCC(C(F)(F)F)C1]

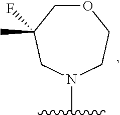 [SMILES: *N1CCOC[C@@](C)(F)C1]

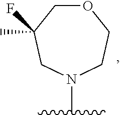 [SMILES: *N1CCOC[C@](C)(F)C1]

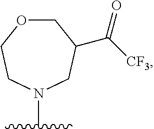 [SMILES: *N1CCOCC(C(=O)C(F)(F)F)C1]

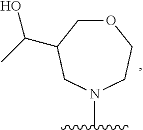 [SMILES: *N1CCOCC(C(C)O)C1]

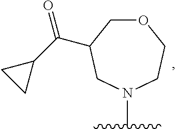 [SMILES: *N1CCOCC(C(=O)C2CC2)C1]

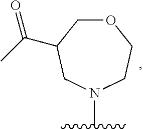 [SMILES: *N1CCOCC(C(C)=O)C1]

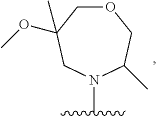

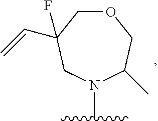 [SMILES: *N1CC(F)(C=C)COCC1C]

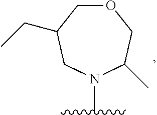 [SMILES: *N1CC(CC)COCC1C]

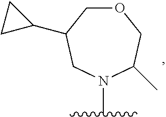 [SMILES: *N1CC(C2CC2)COCC1C]

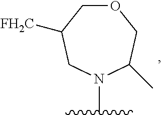

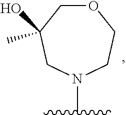 [SMILES: *N1CCOC[C@](C)(O)C1]

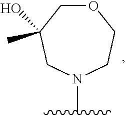 [SMILES: *N1CCOC[C@@](C)(O)C1]

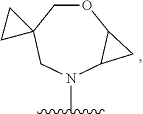 [SMILES: *N1CC2(CC2)COC2CC21]

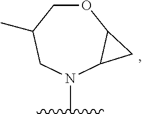 [SMILES: *N1CC(C)COC2CC21]

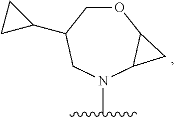 [SMILES: *N1CC(C2CC2)COC2CC21]

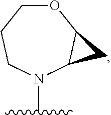 [SMILES: *N1CCCO[C@H]2C[C@H]21]

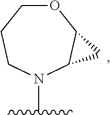 [SMILES: *N1CCCO[C@@H]2C[C@@H]21]

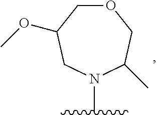

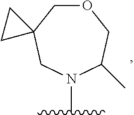 [SMILES: *N1CC2(CC2)COCC1C]

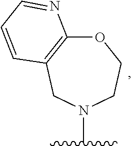 [SMILES: *N1CCOc2ncccc2C1]

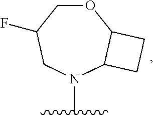 [SMILES: *N1CC(F)COC2CCC21]

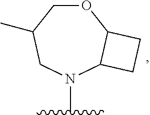 [SMILES: *N1CC(C)COC2CCC21]

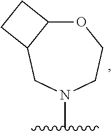 [SMILES: *N1CCOC2CCC2C1]

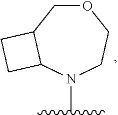 [SMILES: *N1CCOCC2CCC21]

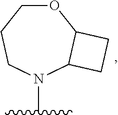 [SMILES: *N1CCCOC2CCC21]

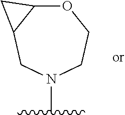 [SMILES: *N1CCOC2CC2C1]

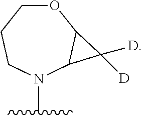

[14]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [13], wherein, the moiety of

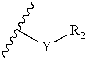

is selected from

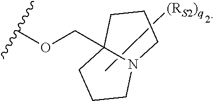

[15]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [14], wherein, the compound is selected from any one of the following formulas:

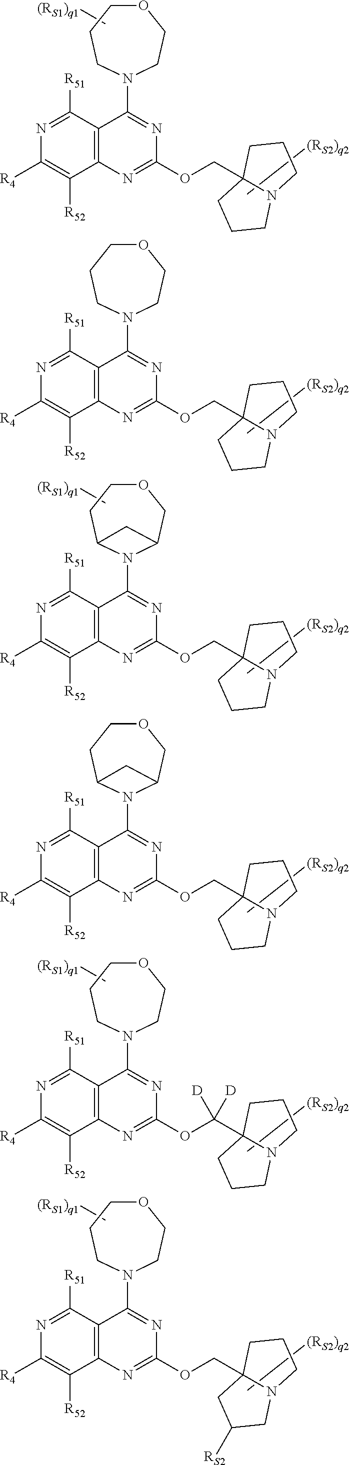

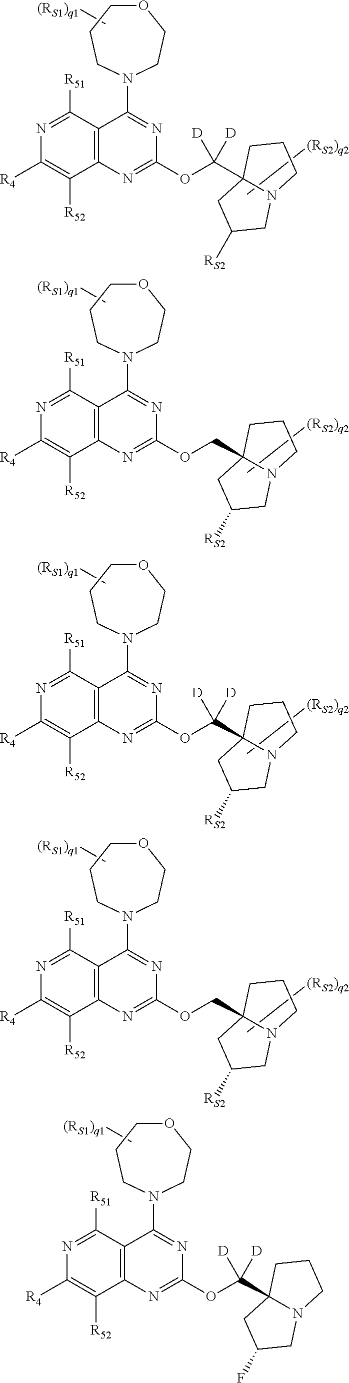

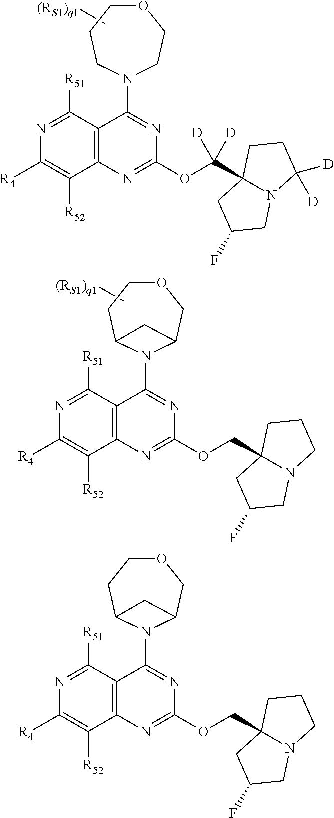

p is selected from 0, 1, 2 or 3.

[16]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [15], wherein,
R_{S2} at each occurrence is independently selected from halogen, —C₁₋₆alkyl, haloC₁₋₆alkyl, haloC₁₋₆alkoxy, —C₂₋₃alkenyl, —CN, —N(R₆₇)₂, —OR₆₇, —SR₆₇, —C(═O)R₆₈, —C(═O)OR₆₇, —OC(═O)R₆₈, —C(═O)N(R₆₇)₂, —NR₆₇C(═O)R₆₈, —OC(═O)OR₆₇, —NR₆₇C(═O)OR₆₇, —OC(═O)N(R₆₇)₂, —NR₆₇C(═O)N(R₆₇)₂, 3-8 membered cycloalkyl, 4-8 membered heterocyclyl containing 1, 2 or 3 heteroatoms selected from N, O or S or

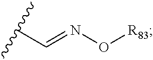

wherein, said —C₁₋₆alkyl is substituted with 1, 2 or 3 substituents selected from halogen, —C₁₋₆alkyl, haloC₁₋₆alkyl, haloC₁₋₆alkoxy, —CN, oxo, —N(R₆₇)₂, —OR₆₇, —C(═O)R₆₈, —C(═O)OR₆₇, —OC(═O)R₆₇, —C(═O)N(R₆₇)₂, —NR₆₇C(═O)R₆₈, —OC(═O)OR₆₇, —NR₆₇C(═O)OR₆₇, —OC(═O)N(R₆₇)₂, —OC(═S)N(R₆₇)₂, —NR₆₇C(═O)N(R₆₇)₂, —NR₆₇S(═O)₂R₆₈, 3-6 membered cycloalkyl or 4-6 membered heterocyclyl; said 4-8 membered heterocyclyl is substituted with 1, 2 or 3 substituents selected from —OR₆₇; said haloC₁₋₆alkyl is substituted with 1, 2 or 3 substituents selected from —OR₆₇ or —C(═O)OR₆₇; said —C₂₋₃alkenyl is substituted with 1 substituents selected from —C(═O)(R₆₇)₂;
Optionally, two R_{S2} together with the carbon atom to which they are both attached form

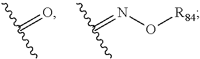

Each of (R₆₇ or R₆₈) is independently selected from hydrogen; —C₁₋₆alkyl; haloC₁₋₆alkyl; 5 membered heteroaryl; cyclopropyl; cyclopentyl; cyclohexyl; 5 membered heterocyclyl; 6 membered heterocyclyl; 5-membered heteroaryl; 6 membered heteroaryl; or —C₁₋₆alkyl substituted with 1, or 2 substituents selected from —OC₁₋₆alkyl, —NHC₁₋₆alkyl, —N(C₁₋₆alkyl)₂ or —C(═O)N(C₁₋₆alkyl)₂; wherein said 5 membered heteroaryl, cyclopropyl, cyclopentyl, cyclohexyl, 5 membered heterocyclyl, 6 membered heterocyclyl, 5-membered heteroaryl or 6 membered heteroaryl is optionally substituted with 1 or 2 substitutents selected from —C₁₋₃alkyl, —OH, —CN, —NH₂, —NH(C₁₋₃alkyl), —N(C₁₋₃alkyl)₂, —OC₁₋₃alkyl or cyclopropyl;
Optionally, two R₆₇ together with the nitrogen atom to which they are both attached form a 3-6 membered heterocyclic ring;
q₂ is selected from 0, 1, 2, 3, 4, 5 or 6; preferably, q₂ is selected from 0, 1, 2, or 3; more preferably, q₂ is selected from 1.

[17]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [16], wherein, R_{S2} at each occurrence is independently selected from —F, —Cl, —CH₃, —CH₂CH₃, —CH₂CH₂CH₃, —CH(CH₃)₂, —CH(CH₃)CH₂CH₃, —CH═CH₂, —C≡CH, —C≡CCH₃, —C≡CD, —CH₂C≡CH, —CHF₂, —CF₃, —CH₂CF₃, —CH₂CHF₂, —CH₂CH₂F, —CH₂CH₂CH₂F, —OH, —CH₂OH, —CH₂CH₂OH, —OCH₃, —OC(CH₃)₂, —OCH₂CH₃, —OCH(CH₃)₂, —OCF₃, —SH, —SCH₃, —SCF₃, —C(═O)CF₃, —C(═O)OCH₂CH₂N(CH₃), —C(═O)NHCH₂CH₂N(CH₃), —CN, —NH₂, —N(CH₃)₂, —NHCH₂CH₃, —CH₂—N(CH₃)₂, —N(CH₃)CH₂CH₂OCH₃, —NHC(═O)CH₃, —NHC(═O)OCH₃, —SCH₂C(═O)N(CH₃)₂, —OC(═O)N(CH₃)₂, —NHC(═O)N(CH₃)₂, —CH₂CH₂CN, —CH₂CH(CH₃)₂, —CH₂OCH₃, —OCHF₂, —CH(CF₃)OCH₃, —C(CH₃)₂OH, —CF(CH₃)₂ or cyclopropyl.

[18]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a drug thereof, a deuterated molecule thereof or a conjugated form thereof of any one of [1] to [17], wherein, the moiety of

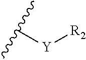

is selected from any moiety in the Table 3:

TABLE 3

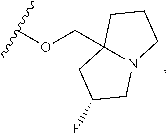 [SMILES: *OCC12CCCN1C[C@H](F)C2]

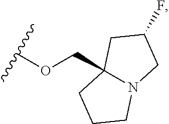 [SMILES: *OC[C@]12CCCN1C[C@@H](F)C2]

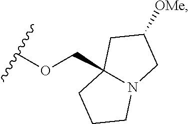 [SMILES: *OC[C@]12CCCN1C[C@@H](OC)C2]

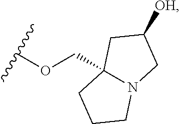 [SMILES: *OC[C@@]12CCCN1C[C@H](O)C2]

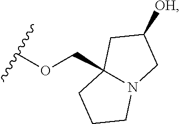 [SMILES: *OC[C@]12CCCN1C[C@H](O)C2]

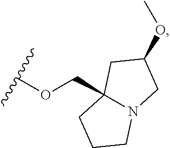 [SMILES: *OC[C@]12CCCN1C[C@H](OC)C2]

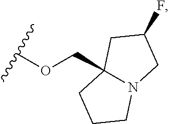 [SMILES: *OC[C@]12CCCN1C[C@H](F)C2]

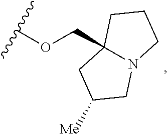 [SMILES: *OC[C@@]12CCCN1C[C@H](C)C2]

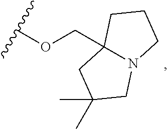 [SMILES: *OCC12CCCN1CC(C)(C)C2]

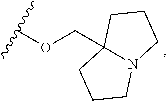 [SMILES: *OCC12CCCN1CCC2]

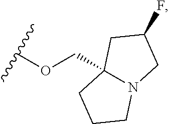 [SMILES: *OC[C@@]12CCCN1C[C@H](F)C2]

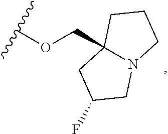 [SMILES: *OC[C@@]12CCCN1C[C@H](F)C2]

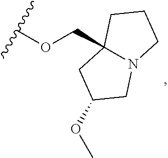 [SMILES: *OC[C@@]12CCCN1C[C@H](OC)C2]

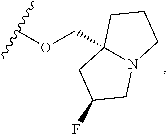 [SMILES: *OC[C@]12CCCN1C[C@@H](F)C2]

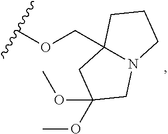 [SMILES: *OCC12CCCN1CC(OC)(OC)C2]

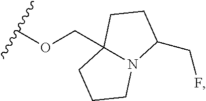 [SMILES: *OCC12CCCN1C(CF)CC2]

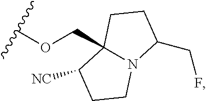 [SMILES: *OC[C@@]12CCC(CF)N1CC[C@@H]2C#N]

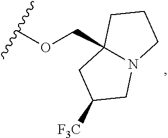 [SMILES: *OC[C@@]12CCCN1C[C@@H](C(F)(F)F)C2]

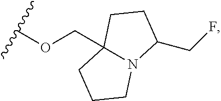 [SMILES: *OCC12CCCN1C(CF)CC2]

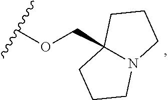

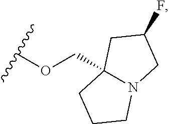 [SMILES: *OC[C@@]12CCCN1C[C@H](F)C2]

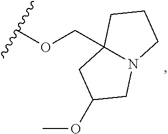 [SMILES: *OCC12CCCN1CC(OC)C2]

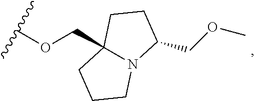 [SMILES: *OC[C@]12CCCN1[C@@H](COC)CC2]

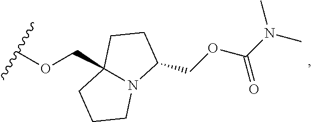 [SMILES: *OC[C@]12CCCN1[C@@H](COC(=O)N(C)C)CC2]

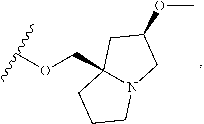 [SMILES: *OC[C@]12CCCN1C[C@H](OC)C2]

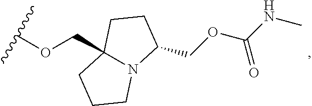 [SMILES: *OC[C@]12CCCN1[C@@H](COC(=O)NC)CC2]

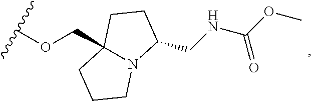 [SMILES: *OC[C@]12CCCN1[C@@H](CNC(=O)OC)CC2]

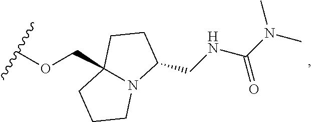 [SMILES: *OC[C@]12CCCN1[C@@H](CNC(=O)N(C)C)CC2]

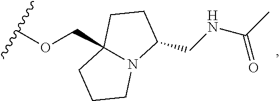 [SMILES: *OC[C@]12CCCN1[C@@H](CNC(C)=O)CC2]

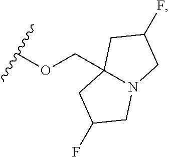 [SMILES: *OCC12CC(F)CN1CC(F)C2]

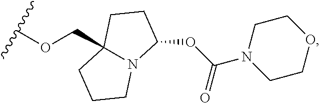 [SMILES: *OC[C@]12CCCN1[C@@H](OC(=O)N1CCOCC1)CC2]

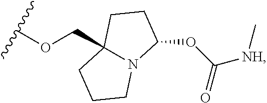 [SMILES: *OC[C@]12CCCN1[C@@H](OC(=O)NC)CC2]

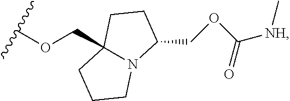 [SMILES: *OC[C@]12CCCN1[C@@H](COC(=O)NC)CC2]

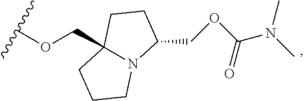 [SMILES: *OC[C@]12CCCN1[C@@H](COC(=O)N(C)C)CC2]

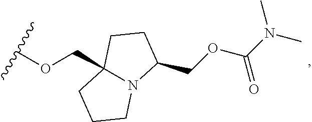 [SMILES: *OC[C@]12CCCN1[C@H](COC(=O)N(C)C)CC2]

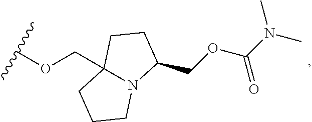 [SMILES: *OCC12CCCN1[C@H](COC(=O)N(C)C)CC2]

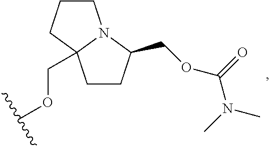 [SMILES: *OCC12CCCN1[C@@H](COC(=O)N(C)C)CC2]

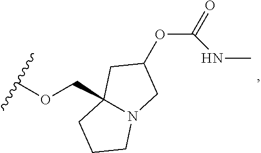 [SMILES: *OC[C@]12CCCN1CC(OC(=O)NC)C2]

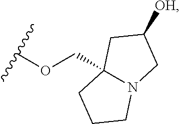 [SMILES: *OC[C@@]12CCCN1C[C@H](O)C2]

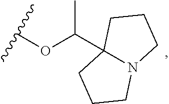 [SMILES: *OC(C)C12CCCN1CCC2]

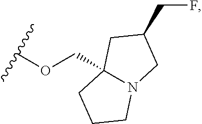 [SMILES: *OC[C@@]12CCCN1C[C@H](CF)C2]

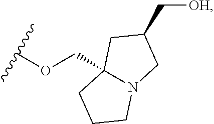 [SMILES: *OC[C@@]12CCCN1C[C@H](CO)C2]

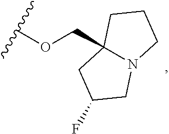 [SMILES: *OC[C@@]12CCCN1C[C@H](F)C2]

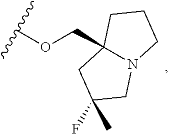 [SMILES: *OC[C@@]12CCCN1C[C@](C)(F)C2]

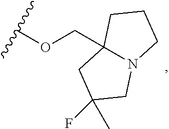 [SMILES: *OCC12CCCN1CC(C)(F)C2]

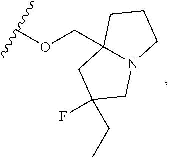 [SMILES: *OCC12CCCN1CC(F)(CC)C2]

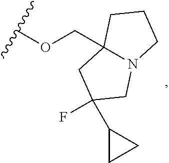 [SMILES: *OCC12CCCN1CC(F)(C1CC1)C2]

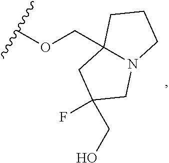 [SMILES: *OCC12CCCN1CC(F)(CO)C2]

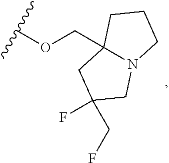 [SMILES: *OCC12CCCN1CC(F)(CF)C2]

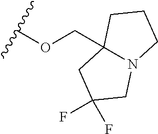 [SMILES: *OCC12CCCN1CC(F)(F)C2]

or

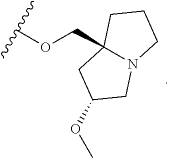 [SMILES: *OC[C@@]12CCCN1C[C@H](OC)C2]

[19]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a drug thereof, a deuterated molecule thereof or a conjugated form thereof of any one of [1] to [17], wherein, the moiety of

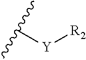

is selected from any moiety in the Table 4:

TABLE 4

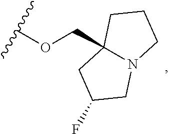 [SMILES: *OC[C@@]12CCCN1C[C@H](F)C2]

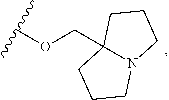 [SMILES: *OCC12CCCN1CCC2]

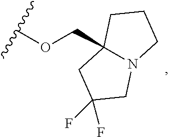 [SMILES: *OC[C@@]12CCCN1CC(F)(F)C2]

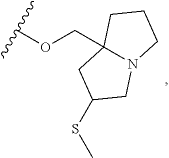 [SMILES: *OCC12CCCN1CC(SC)C2]

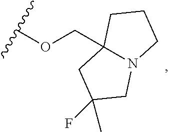 [SMILES: *OCC12CCCN1CC(C)(F)C2]

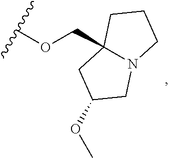 [SMILES: *OC[C@@]12CCCN1C[C@H](OC)C2]

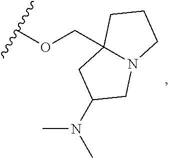 [SMILES: *OCC12CCCN1CC(N(C)C)C2]

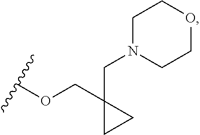 [SMILES: *OCC1(CN2CCOCC2)CC1]

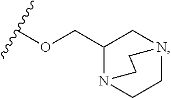 [SMILES: *OCC1CN2CCN1CC2]

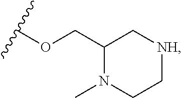 [SMILES: *OCC1CNCCN1C]

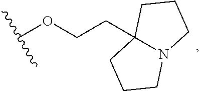 [SMILES: *OCCC12CCCN1CCC2]

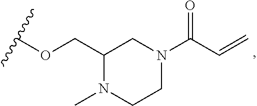 [SMILES: *OCC1CN(C(=O)C=C)CCN1C]

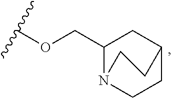 [SMILES: *OCC1CC2CCN1CC2]

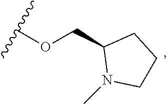 [SMILES: *OC[C@H]1CCCN1C]

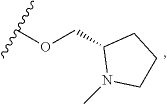 [SMILES: *OC[C@@H]1CCCN1C]

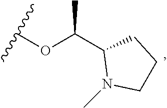 [SMILES: *O[C@@H](C)[C@@H]1CCCN1C]

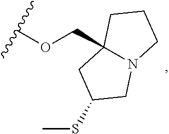 [SMILES: *OC[C@@]12CCCN1C[C@H](SC)C2]

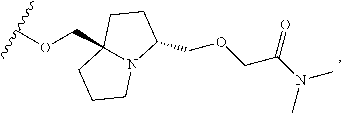 [SMILES: *OC[C@]12CCCN1[C@@H](COCC(=O)N(C)C)CC2]

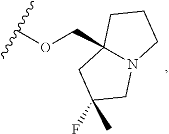 [SMILES: *OC[C@@]12CCCN1C[C@](C)(F)C2]

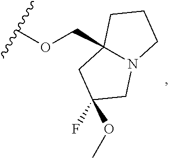 [SMILES: *OC[C@@]12CCCN1C[C@@](F)(OC)C2]

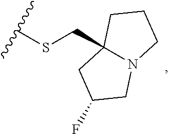 [SMILES: *SC[C@@]12CCCN1C[C@H](F)C2]

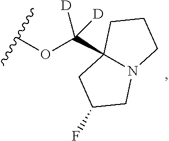 [SMILES: *OC([2H])([2H])[C@@]12CCCN1C[C@H](F)C2]

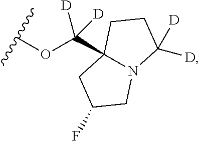

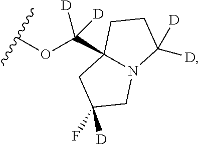

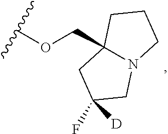 [SMILES: *OC[C@@]12CCCN1C[C@]([2H])(F)C2]

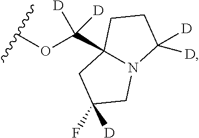

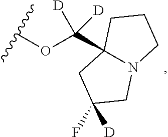 [SMILES: *OC([2H])([2H])[C@@]12CCCN1C[C@]([2H])(F)C2]

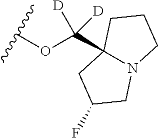 [SMILES: *OC([2H])([2H])[C@@]12CCCN1C[C@H](F)C2]

or

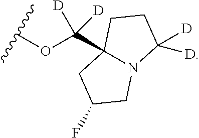

[20]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a drug thereof, a deuterated molecule thereof or a conjugated form thereof of any one of [15] to [17], wherein, the moiety of

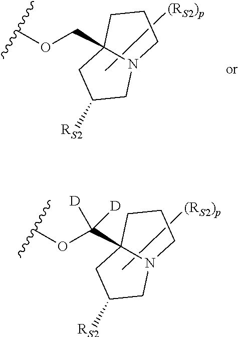

is selected from any moiety in the Table 5:

TABLE 5

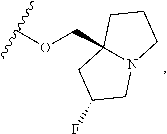 [SMILES: *OC[C@@]12CCCN1C[C@H](F)C2]

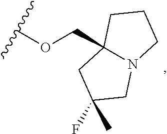 [SMILES: *OC[C@@]12CCCN1C[C@](C)(F)C2]

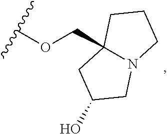 [SMILES: *OC[C@@]12CCCN1C[C@H](O)C2]

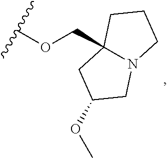 [SMILES: *OC[C@@]12CCCN1C[C@H](OC)C2]

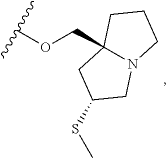 [SMILES: *OC[C@@]12CCCN1C[C@H](SC)C2]

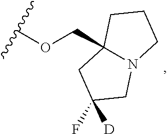 [SMILES: *OC[C@@]12CCCN1C[C@]([2H])(F)C2]

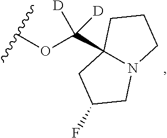 [SMILES: *OC([2H])([2H])[C@@]12CCCN1C[C@H](F)C2]

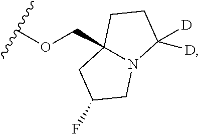

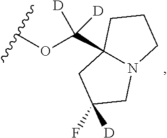 [SMILES: *OC([2H])([2H])[C@@]12CCCN1C[C@]([2H])(F)C2]

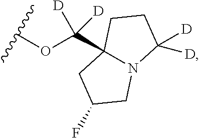

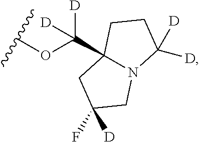

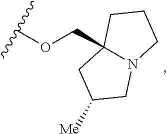 [SMILES: *OC[C@@]12CCCN1C[C@H](C)C2]

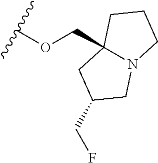 [SMILES: *OC[C@@]12CCCN1C[C@H](CF)C2]

or

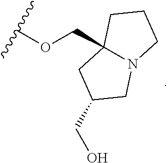 [SMILES: *OC[C@@]12CCCN1C[C@H](CO)C2]

[31]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a drug thereof, a deuterated molecule thereof or a conjugated form thereof of any one of [1] to [20], wherein, R₄ is selected from any moiety in the Table 6:

TABLE 6

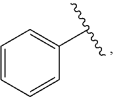 [SMILES: *c1ccccc1]

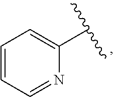 [SMILES: *c1ccccn1]

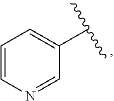 [SMILES: *c1cccnc1]

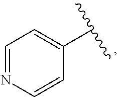 [SMILES: *c1ccncc1]

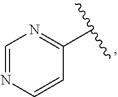 [SMILES: *c1ccncn1]

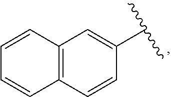 [SMILES: *c1ccc2ccccc2c1]

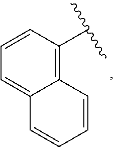 [SMILES: *c1cccc2ccccc12]

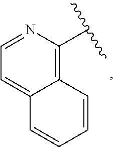 [SMILES: *c1nccc2ccccc12]

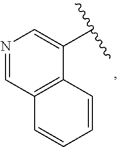 [SMILES: *c1cncc2ccccc12]

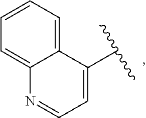 [SMILES: *c1ccnc2ccccc12]

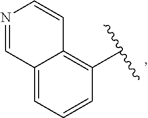 [SMILES: *c1cccc2cnccc12]

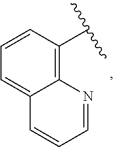 [SMILES: *c1cccc2cccnc12]

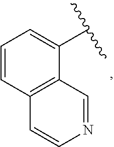 [SMILES: *c1cccc2ccncc12]

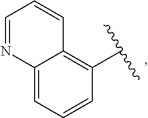 [SMILES: *c1cccc2ncccc12]

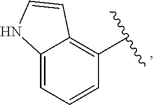 [SMILES: *c1cccc2[nH]ccc12]

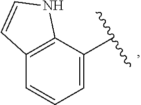 [SMILES: *c1cccc2cc[nH]c12]

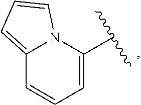 [SMILES: *c1cccc2cccn12]

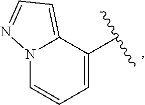 [SMILES: *c1cccn2nccc12]

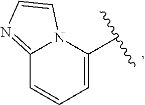 [SMILES: *c1cccc2nccn12]

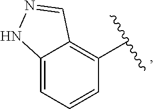 [SMILES: *c1cccc2[nH]ncc12]

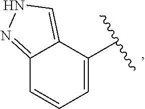 [SMILES: *c1cccc2n[nH]cc12]

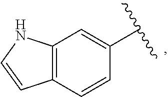 [SMILES: *c1ccc2cc[nH]c2c1]

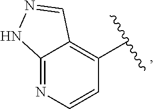 [SMILES: *c1ccnc2[nH]ncc12]

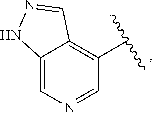 [SMILES: *c1cncc2[nH]ncc12]

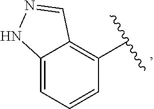 [SMILES: *c1cccc2[nH]ncc12]

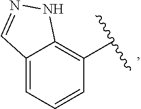 [SMILES: *c1cccc2cn[nH]c12]

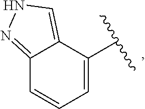 [SMILES: *c1cccc2n[nH]cc12]

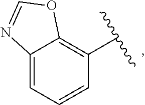 [SMILES: *c1cccc2ncoc12]

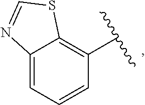 [SMILES: *c1cccc2ncsc12]

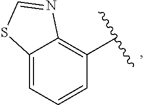 [SMILES: *c1cccc2scnc12]

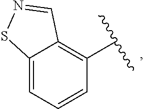 [SMILES: *c1cccc2sncc12]

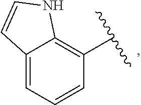 [SMILES: *c1cccc2cc[nH]c12]

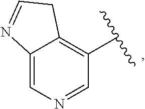 [SMILES: *c1cncc2c1CC=N2]

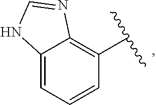 [SMILES: *c1cccc2[nH]cnc12]

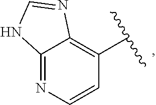 [SMILES: *c1ccnc2[nH]cnc12]

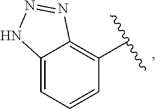 [SMILES: *c1cccc2[nH]nnc12]

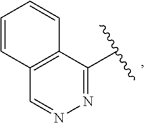 [SMILES: *c1nncc2ccccc12]

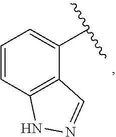 [SMILES: *c1cccc2[nH]ncc12]

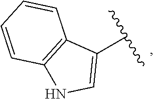 [SMILES: *c1c[nH]c2ccccc12]

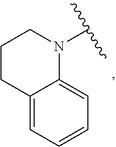 [SMILES: *N1CCCc2ccccc21]

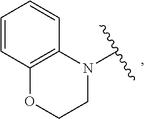 [SMILES: *N1CCOc2ccccc21]

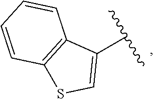 [SMILES: *c1csc2ccccc12]

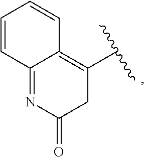 [SMILES: *C1=c2ccccc2=NC(=O)C1]

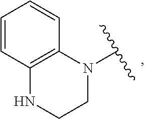 [SMILES: *N1CCNc2ccccc21]

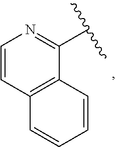 [SMILES: *c1nccc2ccccc12]

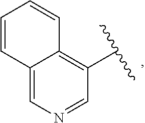 [SMILES: *c1cncc2ccccc12]

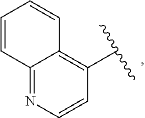 [SMILES: *c1ccnc2ccccc12]

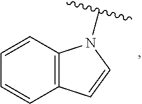 [SMILES: *n1ccc2ccccc21]

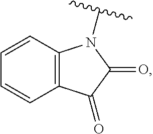 [SMILES: *N1C(=O)C(=O)c2ccccc21]

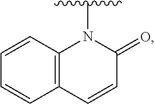 [SMILES: *n1c(=O)ccc2ccccc21]

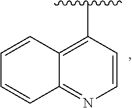 [SMILES: *c1ccnc2ccccc12]

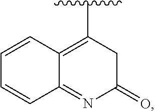 [SMILES: *C1=c2ccccc2=NC(=O)C1]

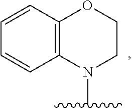 [SMILES: *N1CCOc2ccccc21]

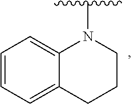 [SMILES: *N1CCCc2ccccc21]

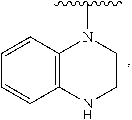 [SMILES: *N1CCNc2ccccc21]

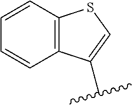 [SMILES: *c1csc2ccccc12]

or

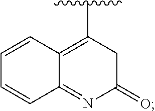

Wherein, each moiety in the Table 6 is independently optionally substituted with 1, 2, 3, 4, 5 or 6 R₄₁.

[22]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [21], wherein, the compound is selected from any one of the following formulas:

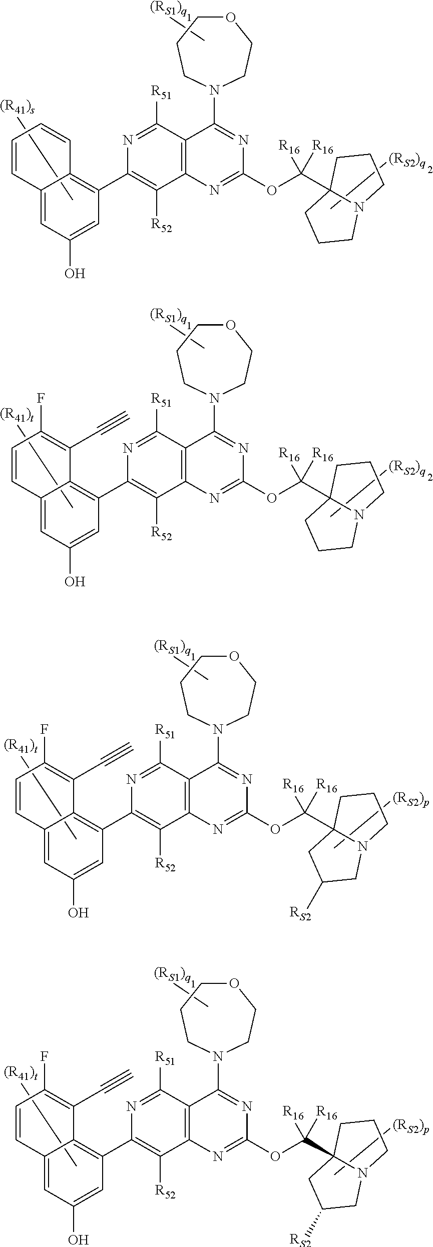

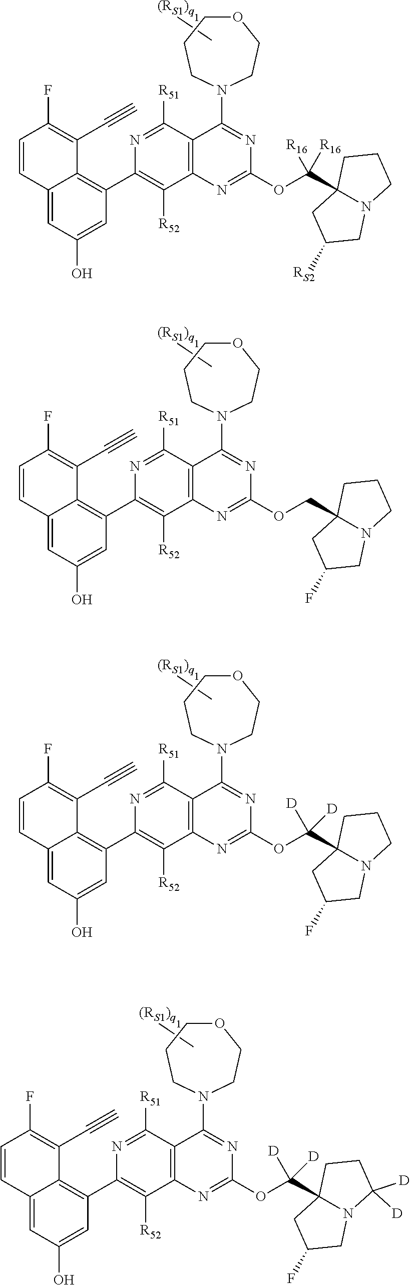

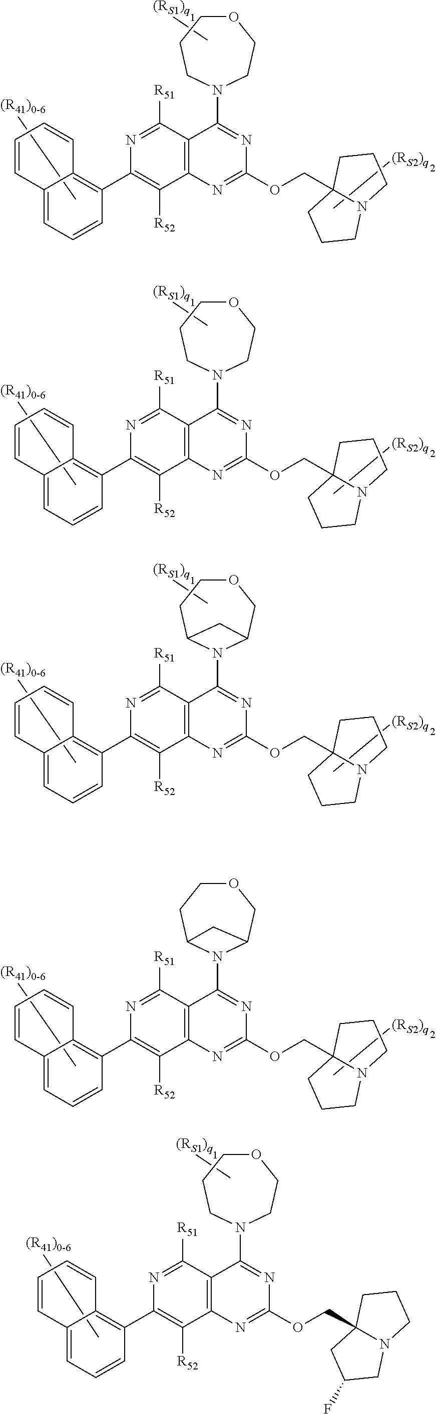

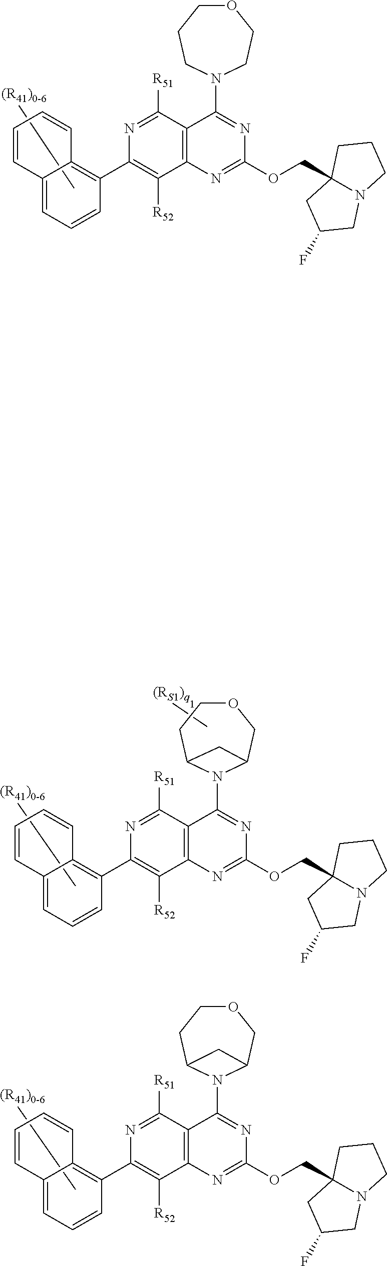

R₁₆ is selected from hydrogen or deuterium;
s is selected from 0, 1, 2, 3, 4, 5 or 6;
t is selected from 0, 1, 2, 3 or 4.

[23]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [22], wherein, R₄₁ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)H, —S(═O)(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, —NH₂, —SH, 3-6 membered cycloalkyl or 3-6 membered heterocyclyl is independently optionally substituted with 1, 2 or 3 R₄₂;
Each of R₄₂ is independently selected from —F; —C₁₋₃alkyl; haloC₁₋₃alkyl; —CN; —OH; —NH₂; —NH(C₁₋₃alkyl); —N(C₁₋₃alkyl)₂; —OC₁₋₃alkyl; 3-6 membered cycloalkyl; or —C₁₋₃alkyl substituted with 1, 2 or 3 substituents selected from —F, haloC₁₋₃alkyl, —CN, —OH, —NH₂, —NH(C₁₋₃alkyl), —N(C₁₋₃alkyl)₂ or —OC₁₋₃alkyl.

[24]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [23], wherein, R₄ is selected from any moiety in the Table 7:

TABLE 7

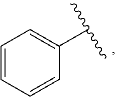 [SMILES: *c1ccccc1]

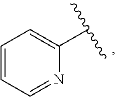 [SMILES: *c1ccccn1]

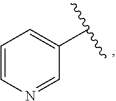 [SMILES: *c1cccnc1]

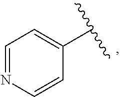 [SMILES: *c1ccncc1]

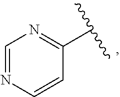 [SMILES: *c1ccncn1]

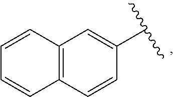 [SMILES: *c1ccc2ccccc2c1]

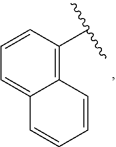 [SMILES: *c1cccc2ccccc12]

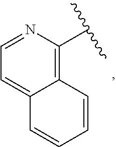 [SMILES: *c1nccc2ccccc12]

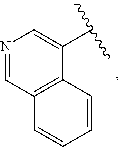 [SMILES: *c1cncc2ccccc12]

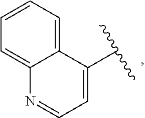 [SMILES: *c1ccnc2ccccc12]

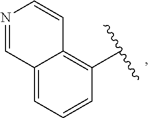 [SMILES: *c1cccc2cnccc12]

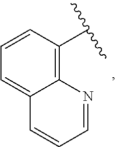 [SMILES: *c1cccc2cccnc12]

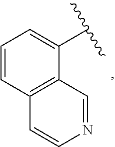 [SMILES: *c1cccc2ccncc12]

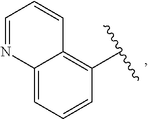 [SMILES: *c1cccc2ncccc12]

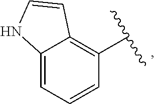 [SMILES: *c1cccc2[nH]ccc12]

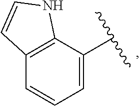 [SMILES: *c1cccc2cc[nH]c12]

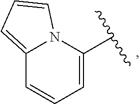 [SMILES: *c1cccc2cccn12]

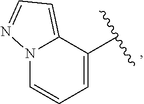 [SMILES: *c1cccn2nccc12]

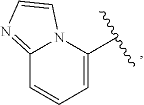 [SMILES: *c1cccc2nccn12]

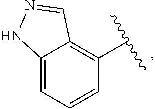 [SMILES: *c1cccc2[nH]ncc12]

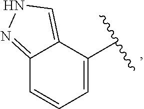 [SMILES: *c1cccc2n[nH]cc12]

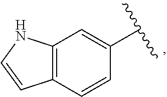 [SMILES: *c1ccc2cc[nH]c2c1]

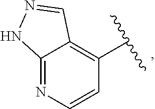 [SMILES: *c1ccnc2[nH]ncc12]

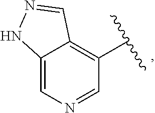 [SMILES: *c1cncc2[nH]ncc12]

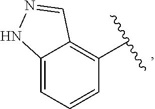 [SMILES: *c1cccc2[nH]ncc12]

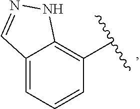 [SMILES: *c1cccc2cn[nH]c12]

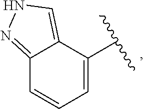 [SMILES: *c1cccc2n[nH]cc12]

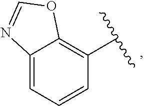 [SMILES: *c1cccc2ncoc12]

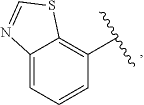 [SMILES: *c1cccc2ncsc12]

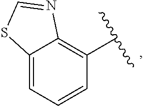 [SMILES: *c1cccc2scnc12]

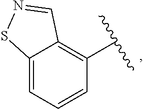 [SMILES: *c1cccc2sncc12]

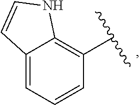 [SMILES: *c1cccc2cc[nH]c12]

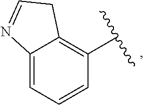 [SMILES: *c1cccc2c1CC=N2]

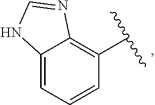 [SMILES: *c1cccc2[nH]cnc12]

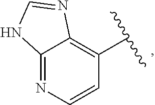 [SMILES: *c1ccnc2[nH]cnc12]

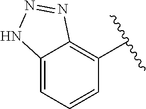 [SMILES: *c1cccc2[nH]nnc12]

or

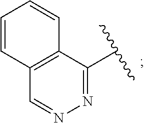

Wherein, said R₄ is independently optionally substituted with 1, 2, 3 or 4 R₄₁;
Each of R₄₁ is independently selected from any moiety in the Table 8:

TABLE 8
—F, —Cl, methyl, ethyl, isopropyl, —CH═CH₂, —C≡CH, —C≡CCH₃, —C≡CD, —CH₂C≡CH, —CHF₂, —CF₃,
—CH₂CF₃, —CH₂CHF₂, —CH₂CH₂F, —CH₂CH₂CH₂F, —OCF₃, —CN, —CH₂CH₂CN, —NH₂, —N(CH₃)₂,
—NHCH₂CH₃, —CH₂—N(CH₃)₂, —OH, —CH₂OH, —CH₂CH₂OH, -OCH₃, —OC(CH₃)₂, —CH₂CH(CH₃)₂,
—CH(CH₃)CH₂CH₃, —CH₂OCH₃, —SH, —SCH₃, —SCF₃, —OCHF₂, —CH(CF₃)OCH₃, —C(CH₃)₂OH, —CF(CH₃)₂,
—OCH(CH₃)₂, cyclopropyl,

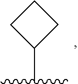 [SMILES: *C1CCC1]

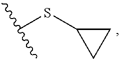 [SMILES: *SC1CC1]

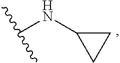 [SMILES: *NC1CC1]

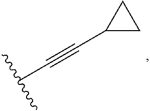 [SMILES: *C#CC1CC1]

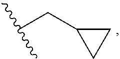 [SMILES: *CC1CC1]

or

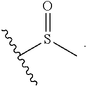 [SMILES: *S(C)=O]

[25]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [24], wherein, R₄ is selected from any moiety in the Table 9:

TABLE 9

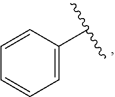 [SMILES: *c1ccccc1]

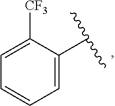 [SMILES: *c1ccccc1C(F)(F)F]

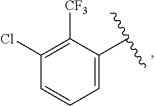 [SMILES: *c1cccc(Cl)c1C(F)(F)F]

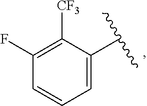 [SMILES: *c1cccc(F)c1C(F)(F)F]

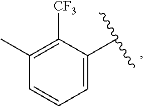 [SMILES: *c1cccc(C)c1C(F)(F)F]

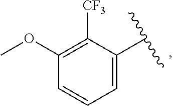 [SMILES: *c1cccc(OC)c1C(F)(F)F]

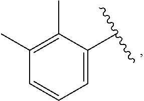 [SMILES: *c1cccc(C)c1C]

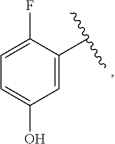 [SMILES: *c1cc(O)ccc1F]

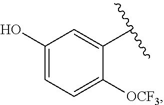 [SMILES: *c1cc(O)ccc1OC(F)(F)F]

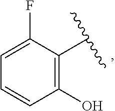 [SMILES: *c1c(O)cccc1F]

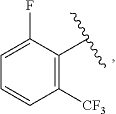 [SMILES: *c1c(F)cccc1C(F)(F)F]

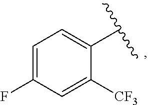 [SMILES: *c1ccc(F)cc1C(F)(F)F]

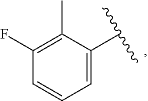 [SMILES: *c1cccc(F)c1C]

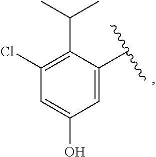 [SMILES: *c1cc(O)cc(Cl)c1C(C)C]

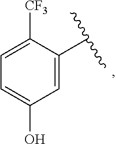 [SMILES: *c1cc(O)ccc1C(F)(F)F]

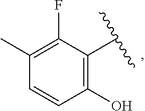 [SMILES: *c1c(O)ccc(C)c1F]

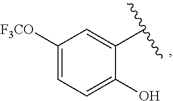 [SMILES: *c1cc(OC(F)(F)F)ccc1O]

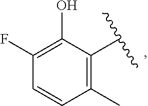 [SMILES: *c1c(C)ccc(F)c1O]

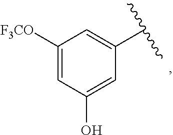 [SMILES: *c1cc(O)cc(OC(F)(F)F)c1]

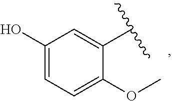 [SMILES: *c1cc(O)ccc1OC]

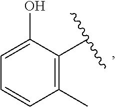 [SMILES: *c1c(C)cccc1O]

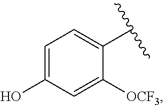 [SMILES: *c1ccc(O)cc1OC(F)(F)F]

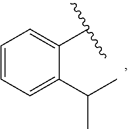 [SMILES: *c1ccccc1C(C)C]

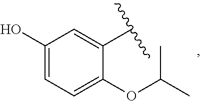 [SMILES: *c1cc(O)ccc1OC(C)C]

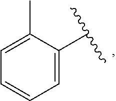 [SMILES: *c1ccccc1C]

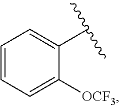 [SMILES: *c1ccccc1OC(F)(F)F]

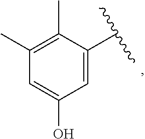 [SMILES: *c1cc(O)cc(C)c1C]

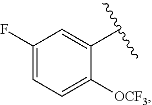 [SMILES: *c1cc(F)ccc1OC(F)(F)F]

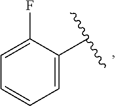 [SMILES: *c1ccccc1F]

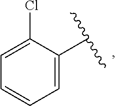 [SMILES: *c1ccccc1Cl]

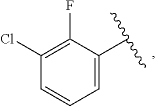 [SMILES: *c1cccc(Cl)c1F]

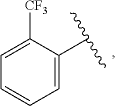 [SMILES: *c1ccccc1C(F)(F)F]

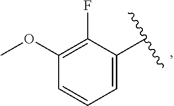 [SMILES: *c1cccc(OC)c1F]

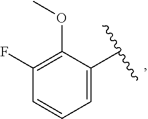 [SMILES: *c1cccc(F)c1OC]

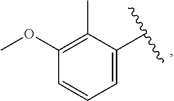 [SMILES: *c1cccc(OC)c1C]

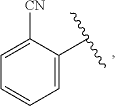 [SMILES: *c1ccccc1C#N]

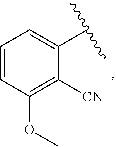 [SMILES: *c1cccc(OC)c1C#N]

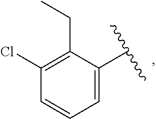 [SMILES: *c1cccc(Cl)c1CC]

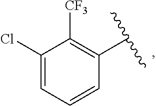 [SMILES: *c1cccc(Cl)c1C(F)(F)F]

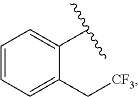 [SMILES: *c1ccccc1CC(F)(F)F]

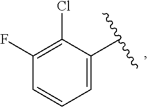 [SMILES: *c1cccc(F)c1Cl]

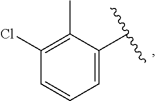 [SMILES: *c1cccc(Cl)c1C]

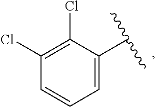 [SMILES: *c1cccc(Cl)c1Cl]

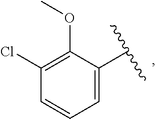 [SMILES: *c1cccc(Cl)c1OC]

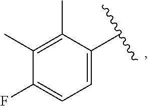 [SMILES: *c1ccc(F)c(C)c1C]

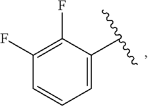 [SMILES: *c1cccc(F)c1F]

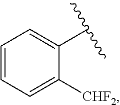 [SMILES: *c1ccccc1C(F)F]

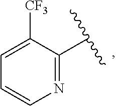 [SMILES: *c1ncccc1C(F)(F)F]

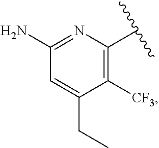 [SMILES: *c1nc(N)cc(CC)c1C(F)(F)F]

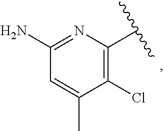 [SMILES: *c1nc(N)cc(C)c1Cl]

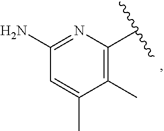 [SMILES: *c1nc(N)cc(C)c1C]

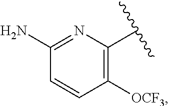 [SMILES: *c1nc(N)ccc1OC(F)(F)F]

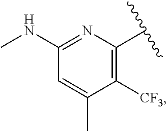 [SMILES: *c1nc(NC)cc(C)c1C(F)(F)F]

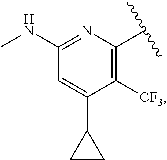 [SMILES: *c1nc(NC)cc(C2CC2)c1C(F)(F)F]

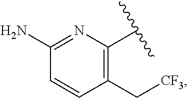 [SMILES: *c1nc(N)ccc1CC(F)(F)F]

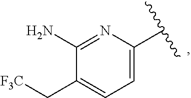 [SMILES: *c1ccc(CC(F)(F)F)c(N)n1]

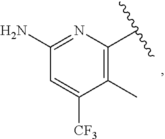 [SMILES: *c1nc(N)cc(C(F)(F)F)c1C]

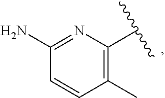 [SMILES: *c1nc(N)ccc1C]

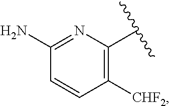 [SMILES: *c1nc(N)ccc1C(F)F]

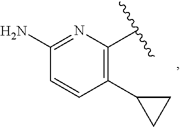 [SMILES: *c1nc(N)ccc1C1CC1]

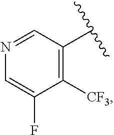 [SMILES: *c1cncc(F)c1C(F)(F)F]

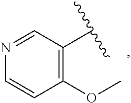 [SMILES: *c1cnccc1OC]

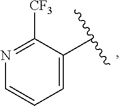 [SMILES: *c1cccnc1C(F)(F)F]

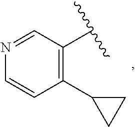 [SMILES: *c1cnccc1C1CC1]

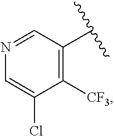 [SMILES: *c1cncc(Cl)c1C(F)(F)F]

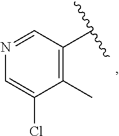 [SMILES: *c1cncc(Cl)c1C]

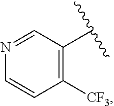 [SMILES: *c1cnccc1C(F)(F)F]

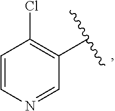 [SMILES: *c1cnccc1Cl]

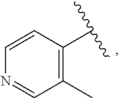 [SMILES: *c1ccncc1C]

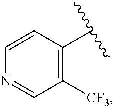 [SMILES: *c1ccncc1C(F)(F)F]

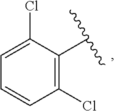 [SMILES: *c1c(Cl)cccc1Cl]

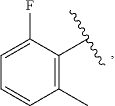 [SMILES: *c1c(C)cccc1F]

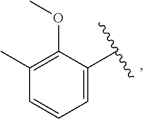 [SMILES: *c1cccc(C)c1OC]

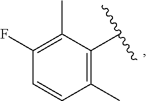 [SMILES: *c1c(C)ccc(F)c1C]

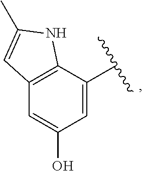 [SMILES: *c1cc(O)cc2cc(C)[nH]c12]

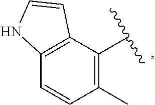 [SMILES: *c1c(C)ccc2[nH]ccc12]

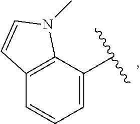 [SMILES: *c1cccc2ccn(C)c12]

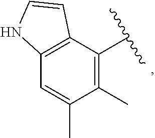 [SMILES: *c1c(C)c(C)cc2[nH]ccc12]

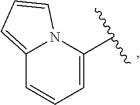 [SMILES: *c1cccc2cccn12]

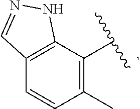 [SMILES: *c1c(C)ccc2cn[nH]c12]

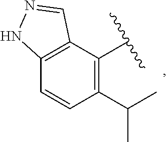 [SMILES: *c1c(C(C)C)ccc2[nH]ncc12]

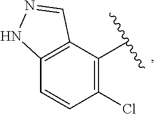 [SMILES: *c1c(Cl)ccc2[nH]ncc12]

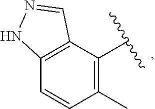 [SMILES: *c1c(C)ccc2[nH]ncc12]

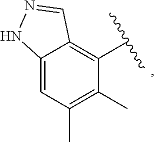 [SMILES: *c1c(C)c(C)cc2[nH]ncc12]

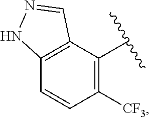 [SMILES: *c1c(C(F)(F)F)ccc2[nH]ncc12]

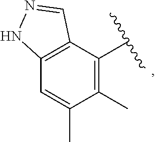 [SMILES: *c1c(C)c(C)cc2[nH]ncc12]

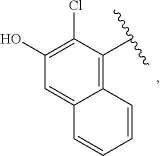 [SMILES: *c1c(Cl)c(O)cc2ccccc12]

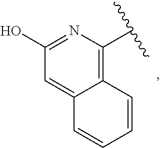 [SMILES: *c1nc(O)cc2ccccc12]

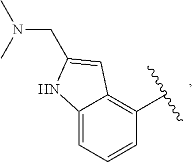 [SMILES: *c1cccc2[nH]c(CN(C)C)cc12]

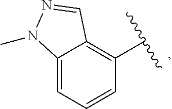 [SMILES: *c1cccc2c1cnn2C]

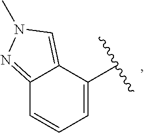 [SMILES: *c1cccc2nn(C)cc12]

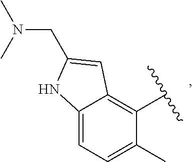

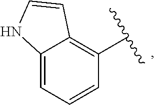 [SMILES: *c1cccc2[nH]ccc12]

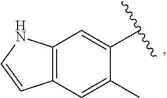 [SMILES: *c1cc2[nH]ccc2cc1C]

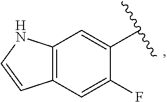 [SMILES: *c1cc2[nH]ccc2cc1F]

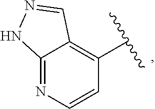 [SMILES: *c1ccnc2[nH]ncc12]

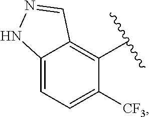 [SMILES: *c1c(C(F)(F)F)ccc2[nH]ncc12]

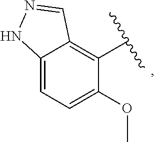 [SMILES: *c1c(OC)ccc2[nH]ncc12]

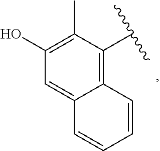 [SMILES: *c1c(C)c(O)cc2ccccc12]

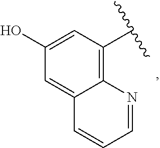 [SMILES: *c1cc(O)cc2cccnc12]

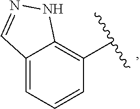 [SMILES: *c1cccc2cn[nH]c12]

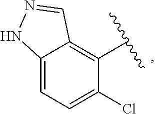 [SMILES: *c1c(Cl)ccc2[nH]ncc12]

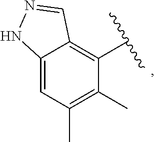 [SMILES: *c1c(C)c(C)cc2[nH]ncc12]

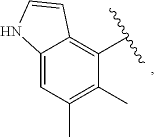 [SMILES: *c1c(C)c(C)cc2[nH]ccc12]

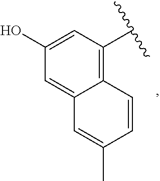 [SMILES: *c1cc(O)cc2cc(C)ccc12]

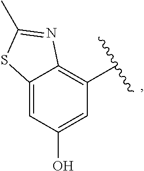 [SMILES: *c1cc(O)cc2sc(C)nc12]

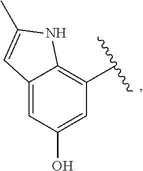 [SMILES: *c1cc(O)cc2cc(C)[nH]c12]

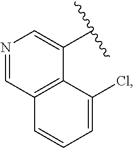 [SMILES: *c1cncc2cccc(Cl)c12]

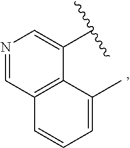 [SMILES: *c1cncc2cccc(C)c12]

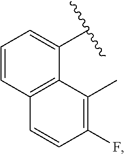 [SMILES: *c1cccc2ccc(F)c(C)c12]

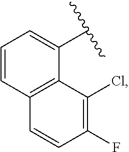 [SMILES: *c1cccc2ccc(F)c(Cl)c12]

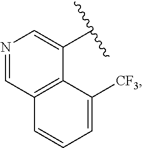 [SMILES: *c1cncc2cccc(C(F)(F)F)c12]

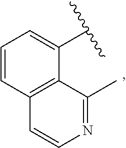 [SMILES: *c1cccc2ccnc(C)c12]

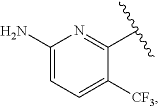 [SMILES: *c1nc(N)ccc1C(F)(F)F]

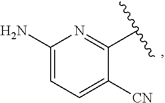 [SMILES: *c1nc(N)ccc1C#N]

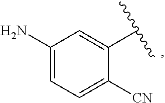 [SMILES: *c1cc(N)ccc1C#N]

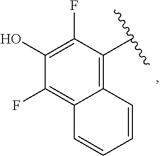 [SMILES: *c1c(F)c(O)c(F)c2ccccc12]

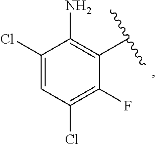 [SMILES: *c1c(N)c(Cl)cc(Cl)c1F]

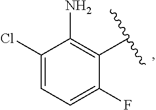 [SMILES: *c1c(F)ccc(Cl)c1N]

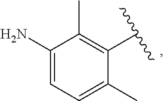 [SMILES: *c1c(C)ccc(N)c1C]

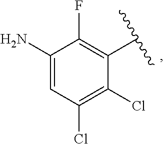 [SMILES: *c1c(F)c(N)cc(Cl)c1Cl]

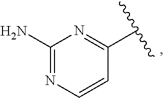 [SMILES: *c1ccnc(N)n1]

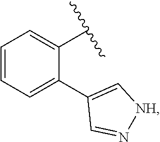 [SMILES: *c1ccccc1-c1cn[nH]c1]

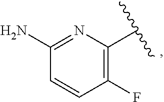 [SMILES: *c1nc(N)ccc1F]

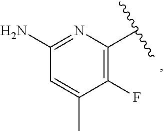 [SMILES: *c1nc(N)cc(C)c1F]

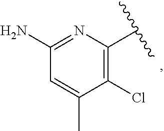 [SMILES: *c1nc(N)cc(C)c1Cl]

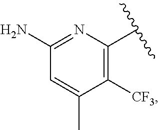 [SMILES: *c1nc(N)cc(C)c1C(F)(F)F]

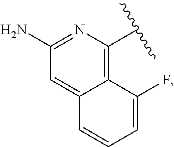 [SMILES: *c1nc(N)cc2cccc(F)c12]

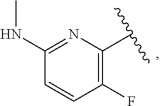 [SMILES: *c1nc(NC)ccc1F]

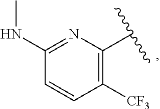 [SMILES: *c1nc(NC)ccc1C(F)(F)F]

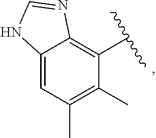 [SMILES: *c1c(C)c(C)cc2[nH]cnc12]

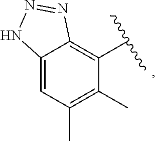 [SMILES: *c1c(C)c(C)cc2[nH]nnc12]

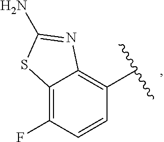 [SMILES: *c1ccc(F)c2sc(N)nc12]

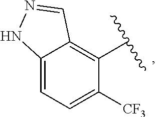 [SMILES: *c1c(C(F)(F)F)ccc2[nH]ncc12]

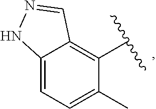 [SMILES: *c1c(C)ccc2[nH]ncc12]

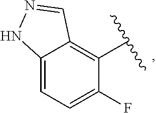 [SMILES: *c1c(F)ccc2[nH]ncc12]

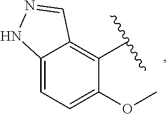 [SMILES: *c1c(OC)ccc2[nH]ncc12]

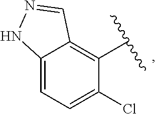 [SMILES: *c1c(Cl)ccc2[nH]ncc12]

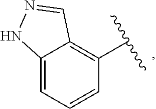 [SMILES: *c1cccc2[nH]ncc12]

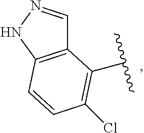 [SMILES: *c1c(Cl)ccc2[nH]ncc12]

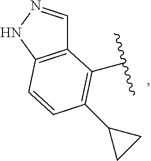 [SMILES: *c1c(C2CC2)ccc2[nH]ncc12]

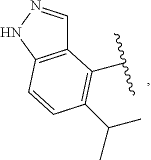 [SMILES: *c1c(C(C)C)ccc2[nH]ncc12]

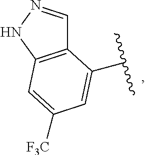 [SMILES: *c1cc(C(F)(F)F)cc2[nH]ncc12]

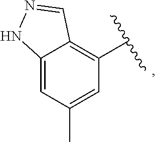 [SMILES: *c1cc(C)cc2[nH]ncc12]

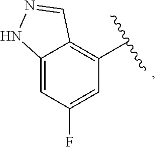 [SMILES: *c1cc(F)cc2[nH]ncc12]

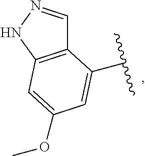 [SMILES: *c1cc(OC)cc2[nH]ncc12]

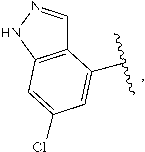 [SMILES: *c1cc(Cl)cc2[nH]ncc12]

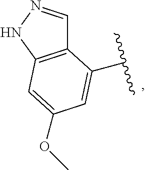 [SMILES: *c1cc(OC)cc2[nH]ncc12]

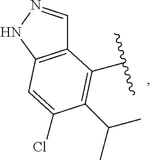 [SMILES: *c1c(C(C)C)c(Cl)cc2[nH]ncc12]

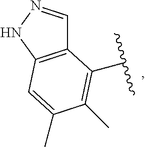 [SMILES: *c1c(C)c(C)cc2[nH]ncc12]

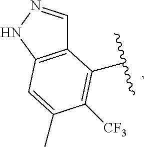 [SMILES: *c1c(C(F)(F)F)c(C)cc2[nH]ncc12]

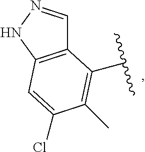 [SMILES: *c1c(C)c(Cl)cc2[nH]ncc12]

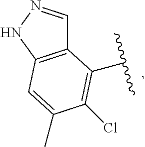 [SMILES: *c1c(Cl)c(C)cc2[nH]ncc12]

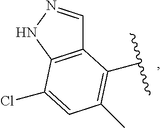 [SMILES: *c1c(C)cc(Cl)c2[nH]ncc12]

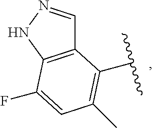 [SMILES: *c1c(C)cc(F)c2[nH]ncc12]

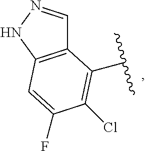 [SMILES: *c1c(Cl)c(F)cc2[nH]ncc12]

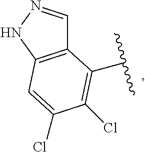 [SMILES: *c1c(Cl)c(Cl)cc2[nH]ncc12]

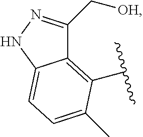 [SMILES: *c1c(C)ccc2[nH]nc(CO)c12]

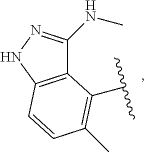 [SMILES: *c1c(C)ccc2[nH]nc(NC)c12]

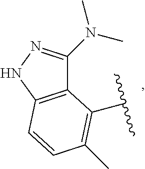 [SMILES: *c1c(C)ccc2[nH]nc(N(C)C)c12]

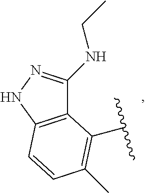 [SMILES: *c1c(C)ccc2[nH]nc(NCC)c12]

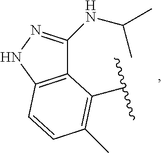 [SMILES: *c1c(C)ccc2[nH]nc(NC(C)C)c12]

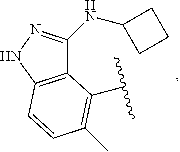 [SMILES: *c1c(C)ccc2[nH]nc(NC3CCC3)c12]

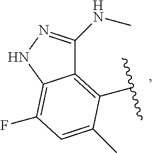 [SMILES: *c1c(C)cc(F)c2[nH]nc(NC)c12]

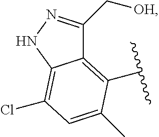 [SMILES: *c1c(C)cc(Cl)c2[nH]nc(CO)c12]

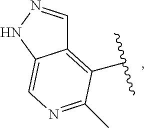 [SMILES: *c1c(C)ncc2[nH]ncc12]

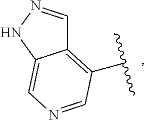 [SMILES: *c1cncc2[nH]ncc12]

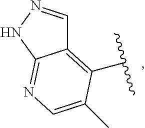 [SMILES: *c1c(C)cnc2[nH]ncc12]

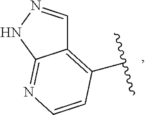 [SMILES: *c1ccnc2[nH]ncc12]

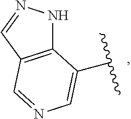 [SMILES: *c1cncc2cn[nH]c12]

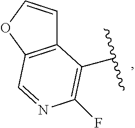 [SMILES: *c1c(F)ncc2occc12]

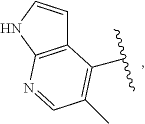 [SMILES: *c1c(C)cnc2[nH]ccc12]

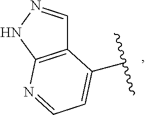 [SMILES: *c1ccnc2[nH]ncc12]

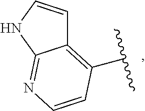 [SMILES: *c1ccnc2[nH]ccc12]

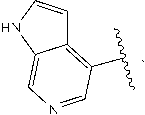 [SMILES: *c1cncc2[nH]ccc12]

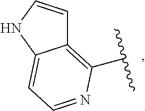 [SMILES: *c1nccc2[nH]ccc12]

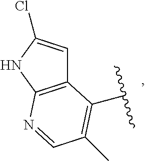 [SMILES: *c1c(C)cnc2[nH]c(Cl)cc12]

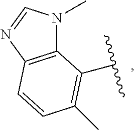 [SMILES: *c1c(C)ccc2ncn(C)c12]

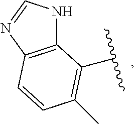 [SMILES: *c1c(C)ccc2nc[nH]c12]

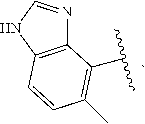 [SMILES: *c1c(C)ccc2[nH]cnc12]

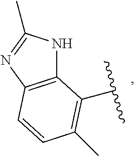 [SMILES: *c1c(C)ccc2nc(C)[nH]c12]

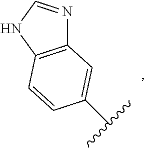 [SMILES: *c1ccc2[nH]cnc2c1]

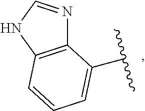 [SMILES: *c1cccc2[nH]cnc12]

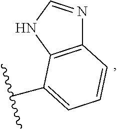 [SMILES: *c1cccc2nc[nH]c12]

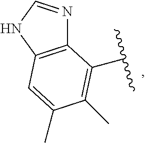 [SMILES: *c1c(C)c(C)cc2[nH]cnc12]

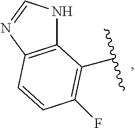 [SMILES: *c1c(F)ccc2nc[nH]c12]

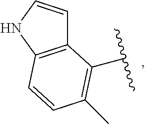 [SMILES: *c1c(C)ccc2[nH]ccc12]

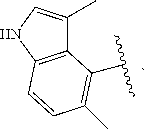 [SMILES: *c1c(C)ccc2[nH]cc(C)c12]

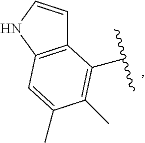 [SMILES: *c1c(C)c(C)cc2[nH]ccc12]

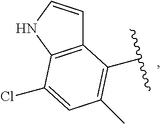 [SMILES: *c1c(C)cc(Cl)c2[nH]ccc12]

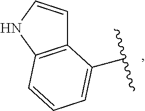 [SMILES: *c1cccc2[nH]ccc12]

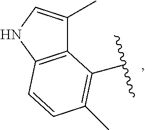 [SMILES: *c1c(C)ccc2[nH]cc(C)c12]

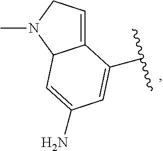 [SMILES: *C1=CC(N)=CC2C1=CCN2C]

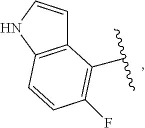 [SMILES: *c1c(F)ccc2[nH]ccc12]

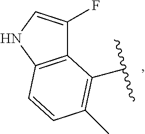 [SMILES: *c1c(C)ccc2[nH]cc(F)c12]

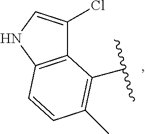 [SMILES: *c1c(C)ccc2[nH]cc(Cl)c12]

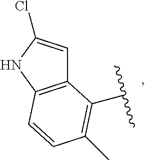 [SMILES: *c1c(C)ccc2[nH]c(Cl)cc12]

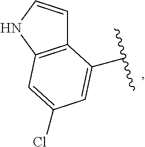 [SMILES: *c1cc(Cl)cc2[nH]ccc12]

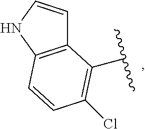 [SMILES: *c1c(Cl)ccc2[nH]ccc12]

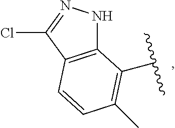 [SMILES: *c1c(C)ccc2c(Cl)n[nH]c12]

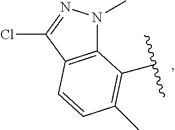 [SMILES: *c1c(C)ccc2c(Cl)nn(C)c12]

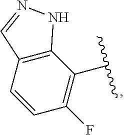 [SMILES: *c1c(F)ccc2cn[nH]c12]

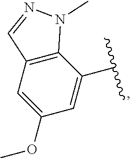 [SMILES: *c1cc(OC)cc2cnn(C)c12]

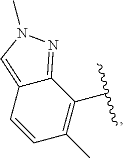 [SMILES: *c1c(C)ccc2cn(C)nc12]

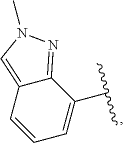 [SMILES: *c1cccc2cn(C)nc12]

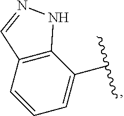 [SMILES: *c1cccc2cn[nH]c12]

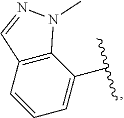 [SMILES: *c1cccc2cnn(C)c12]

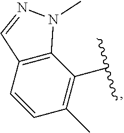 [SMILES: *c1c(C)ccc2cnn(C)c12]

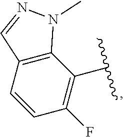 [SMILES: *c1c(F)ccc2cnn(C)c12]

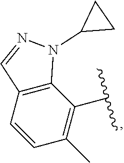 [SMILES: *c1c(C)ccc2cnn(C3CC3)c12]

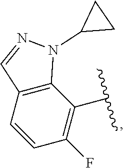 [SMILES: *c1c(F)ccc2cnn(C3CC3)c12]

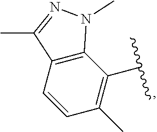 [SMILES: *c1c(C)ccc2c(C)nn(C)c12]

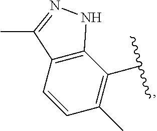 [SMILES: *c1c(C)ccc2c(C)n[nH]c12]

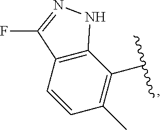 [SMILES: *c1c(C)ccc2c(F)n[nH]c12]

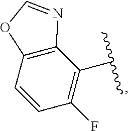 [SMILES: *c1c(F)ccc2ocnc12]

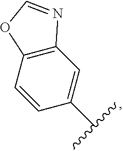 [SMILES: *c1ccc2ocnc2c1]

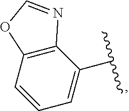 [SMILES: *c1cccc2ocnc12]

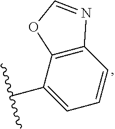 [SMILES: *c1cccc2ncoc12]

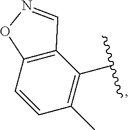 [SMILES: *c1c(C)ccc2oncc12]

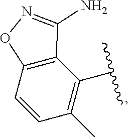 [SMILES: *c1c(C)ccc2onc(N)c12]

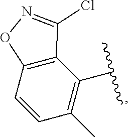 [SMILES: *c1c(C)ccc2onc(Cl)c12]

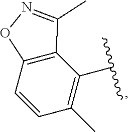 [SMILES: *c1c(C)ccc2onc(C)c12]

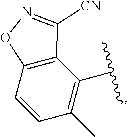 [SMILES: *c1c(C)ccc2onc(C#N)c12]

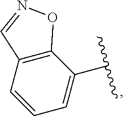 [SMILES: *c1cccc2cnoc12]

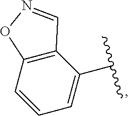 [SMILES: *c1cccc2oncc12]

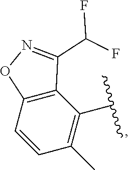 [SMILES: *c1c(C)ccc2onc(C(F)F)c12]

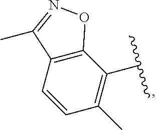 [SMILES: *c1c(C)ccc2c(C)noc12]

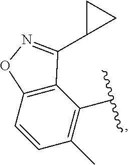 [SMILES: *c1c(C)ccc2onc(C3CC3)c12]

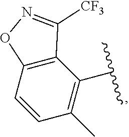 [SMILES: *c1c(C)ccc2onc(C(F)(F)F)c12]

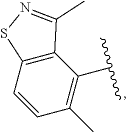 [SMILES: *c1c(C)ccc2snc(C)c12]

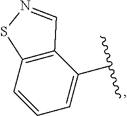 [SMILES: *c1cccc2sncc12]

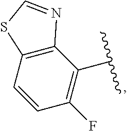 [SMILES: *c1c(F)ccc2scnc12]

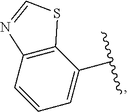 [SMILES: *c1cccc2ncsc12]

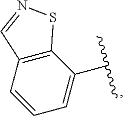 [SMILES: *c1cccc2cnsc12]

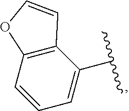 [SMILES: *c1cccc2occc12]

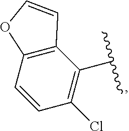 [SMILES: *c1c(Cl)ccc2occc12]

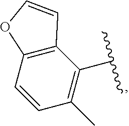 [SMILES: *c1c(C)ccc2occc12]

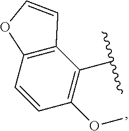

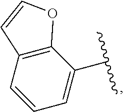 [SMILES: *c1cccc2ccoc12]

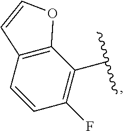 [SMILES: *c1c(F)ccc2ccoc12]

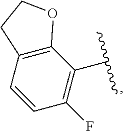 [SMILES: *c1c(F)ccc2c1OCC2]

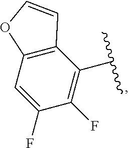 [SMILES: *c1c(F)c(F)cc2occc12]

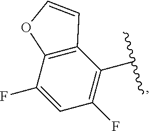 [SMILES: *c1c(F)cc(F)c2occc12]

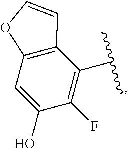 [SMILES: *c1c(F)c(O)cc2occc12]

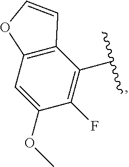 [SMILES: *c1c(F)c(OC)cc2occc12]

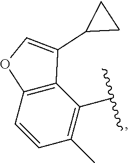 [SMILES: *c1c(C)ccc2occ(C3CC3)c12]

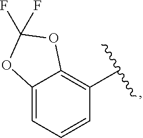 [SMILES: *c1cccc2c1OC(F)(F)O2]

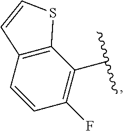 [SMILES: *c1c(F)ccc2ccsc12]

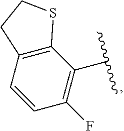 [SMILES: *c1c(F)ccc2c1SCC2]

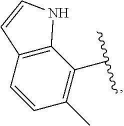 [SMILES: *c1c(C)ccc2cc[nH]c12]

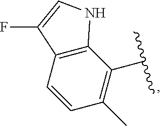 [SMILES: *c1c(C)ccc2c(F)c[nH]c12]

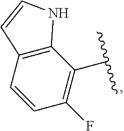 [SMILES: *c1c(F)ccc2cc[nH]c12]

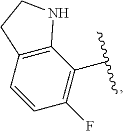 [SMILES: *c1c(F)ccc2c1NCC2]

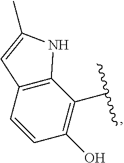 [SMILES: *c1c(O)ccc2cc(C)[nH]c12]

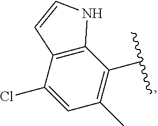 [SMILES: *c1c(C)cc(Cl)c2cc[nH]c12]

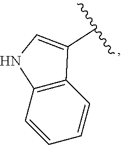 [SMILES: *c1c[nH]c2ccccc12]

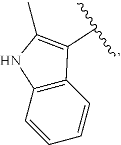 [SMILES: *c1c(C)[nH]c2ccccc12]

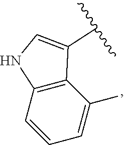

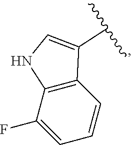 [SMILES: *c1c[nH]c2c(F)cccc12]

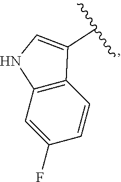 [SMILES: *c1c[nH]c2cc(F)ccc12]

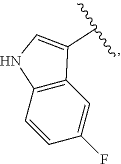 [SMILES: *c1c[nH]c2ccc(F)cc12]

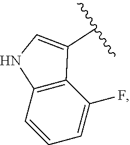 [SMILES: *c1c[nH]c2cccc(F)c12]

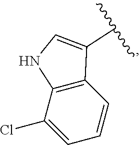 [SMILES: *c1c[nH]c2c(Cl)cccc12]

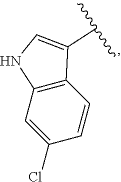 [SMILES: *c1c[nH]c2cc(Cl)ccc12]

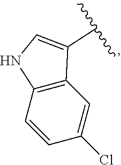 [SMILES: *c1c[nH]c2ccc(Cl)cc12]

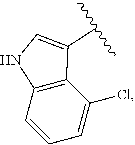 [SMILES: *c1c[nH]c2cccc(Cl)c12]

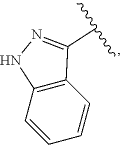 [SMILES: *c1n[nH]c2ccccc12]

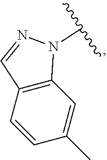 [SMILES: *n1ncc2ccc(C)cc21]

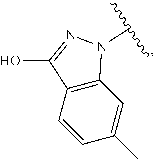 [SMILES: *n1nc(O)c2ccc(C)cc21]

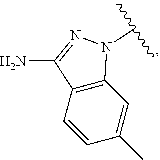 [SMILES: *n1nc(N)c2ccc(C)cc21]

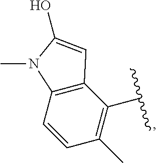 [SMILES: *c1c(C)ccc2c1cc(O)n2C]

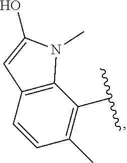 [SMILES: *c1c(C)ccc2cc(O)n(C)c12]

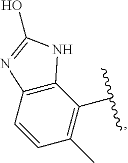 [SMILES: *c1c(C)ccc2nc(O)[nH]c12]

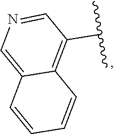 [SMILES: *c1cncc2ccccc12]

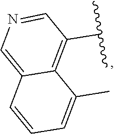 [SMILES: *c1cncc2cccc(C)c12]

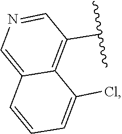 [SMILES: *c1cncc2cccc(Cl)c12]

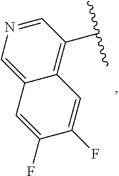 [SMILES: *c1cncc2cc(F)c(F)cc12]

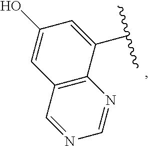 [SMILES: *c1cc(O)cc2cncnc12]

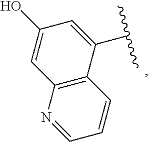 [SMILES: *c1cc(O)cc2ncccc12]

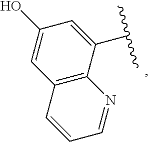 [SMILES: *c1cc(O)cc2cccnc12]

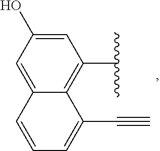 [SMILES: *c1cc(O)cc2cccc(C#C)c12]

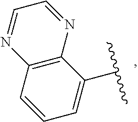 [SMILES: *c1cccc2nccnc12]

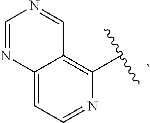 [SMILES: *c1nccc2ncncc12]

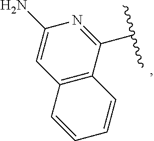 [SMILES: *c1nc(N)cc2ccccc12]

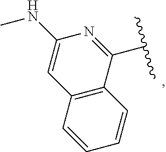 [SMILES: *c1nc(NC)cc2ccccc12]

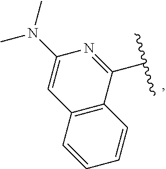 [SMILES: *c1nc(N(C)C)cc2ccccc12]

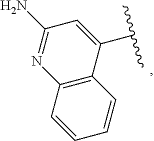 [SMILES: *c1cc(N)nc2ccccc12]

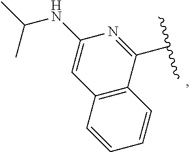 [SMILES: *c1nc(NC(C)C)cc2ccccc12]

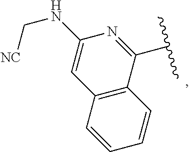 [SMILES: *c1nc(NCC#N)cc2ccccc12]

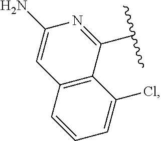 [SMILES: *c1nc(N)cc2cccc(Cl)c12]

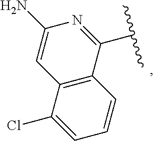 [SMILES: *c1nc(N)cc2c(Cl)cccc12]

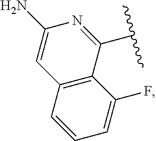 [SMILES: *c1nc(N)cc2cccc(F)c12]

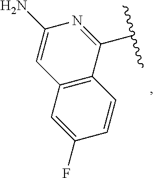 [SMILES: *c1nc(N)cc2cc(F)ccc12]

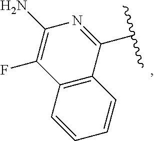 [SMILES: *c1nc(N)c(F)c2ccccc12]

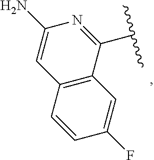 [SMILES: *c1nc(N)cc2ccc(F)cc12]

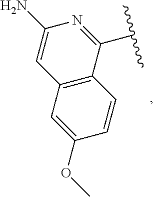 [SMILES: *c1nc(N)cc2cc(OC)ccc12]

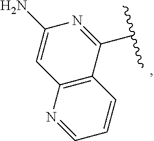 [SMILES: *c1nc(N)cc2ncccc12]

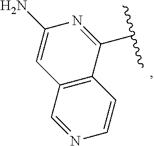 [SMILES: *c1nc(N)cc2cnccc12]

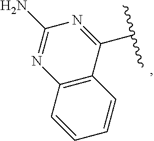 [SMILES: *c1nc(N)nc2ccccc12]

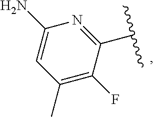 [SMILES: *c1nc(N)cc(C)c1F]

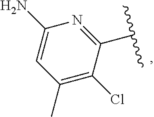 [SMILES: *c1nc(N)cc(C)c1Cl]

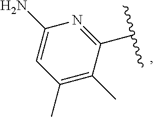 [SMILES: *c1nc(N)cc(C)c1C]

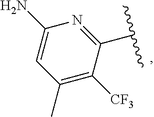 [SMILES: *c1nc(N)cc(C)c1C(F)(F)F]

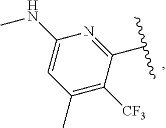 [SMILES: *c1nc(NC)cc(C)c1C(F)(F)F]

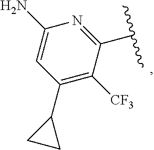 [SMILES: *c1nc(N)cc(C2CC2)c1C(F)(F)F]

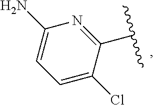 [SMILES: *c1nc(N)ccc1Cl]

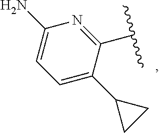 [SMILES: *c1nc(N)ccc1C1CC1]

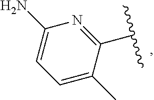 [SMILES: *c1nc(N)ccc1C]

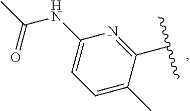 [SMILES: *c1nc(NC(C)=O)ccc1C]

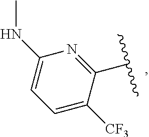 [SMILES: *c1nc(NC)ccc1C(F)(F)F]

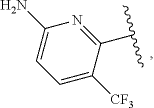 [SMILES: *c1nc(N)ccc1C(F)(F)F]

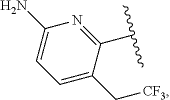 [SMILES: *c1nc(N)ccc1CC(F)(F)F]

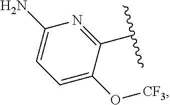 [SMILES: *c1nc(N)ccc1OC(F)(F)F]

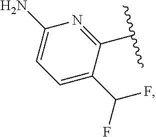 [SMILES: *c1nc(N)ccc1C(F)F]

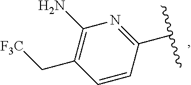 [SMILES: *c1ccc(CC(F)(F)F)c(N)n1]

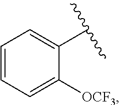 [SMILES: *c1ccccc1OC(F)(F)F]

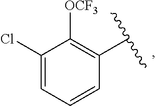 [SMILES: *c1cccc(Cl)c1OC(F)(F)F]

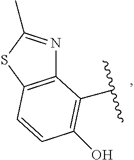 [SMILES: *c1c(O)ccc2sc(C)nc12]

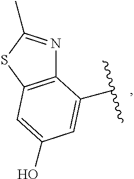 [SMILES: *c1cc(O)cc2sc(C)nc12]

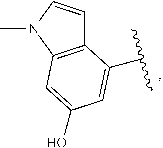 [SMILES: *c1cc(O)cc2c1ccn2C]

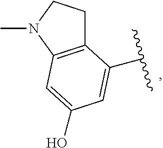 [SMILES: *c1cc(O)cc2c1CCN2C]

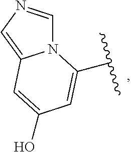 [SMILES: *c1cc(O)cc2cncn12]

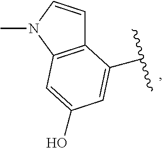 [SMILES: *c1cc(O)cc2c1ccn2C]

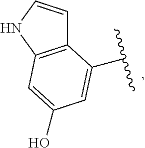 [SMILES: *c1cc(O)cc2[nH]ccc12]

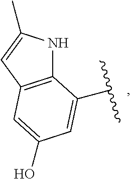 [SMILES: *c1cc(O)cc2cc(C)[nH]c12]

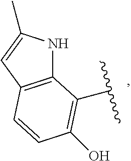 [SMILES: *c1c(O)ccc2cc(C)[nH]c12]

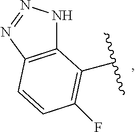 [SMILES: *c1c(F)ccc2nn[nH]c12]

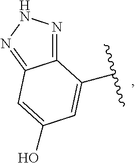 [SMILES: *c1cc(O)cc2n[nH]nc12]

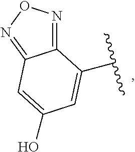 [SMILES: *c1cc(O)cc2nonc12]

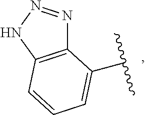 [SMILES: *c1cccc2[nH]nnc12]

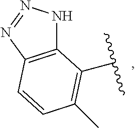 [SMILES: *c1c(C)ccc2nn[nH]c12]

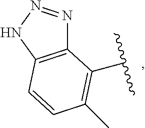 [SMILES: *c1c(C)ccc2[nH]nnc12]

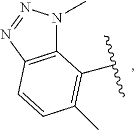 [SMILES: *c1c(C)ccc2nnn(C)c12]

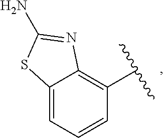 [SMILES: *c1cccc2sc(N)nc12]

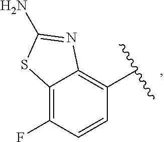 [SMILES: *c1ccc(F)c2sc(N)nc12]

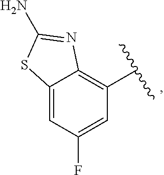 [SMILES: *c1cc(F)cc2sc(N)nc12]

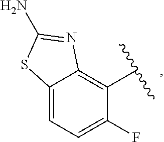 [SMILES: *c1c(F)ccc2sc(N)nc12]

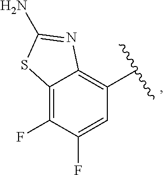 [SMILES: *c1cc(F)c(F)c2sc(N)nc12]

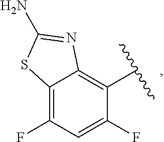 [SMILES: *c1c(F)cc(F)c2sc(N)nc12]

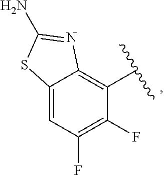 [SMILES: *c1c(F)c(F)cc2sc(N)nc12]

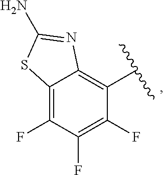 [SMILES: *c1c(F)c(F)c(F)c2sc(N)nc12]

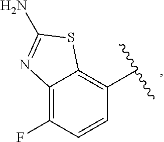 [SMILES: *c1ccc(F)c2nc(N)sc12]

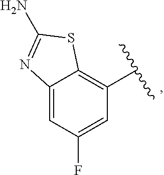 [SMILES: *c1cc(F)cc2nc(N)sc12]

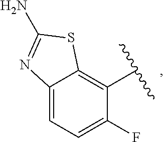 [SMILES: *c1c(F)ccc2nc(N)sc12]

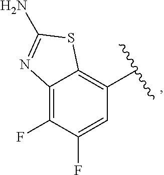 [SMILES: *c1cc(F)c(F)c2nc(N)sc12]

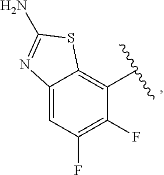 [SMILES: *c1c(F)c(F)cc2nc(N)sc12]

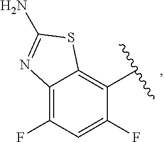 [SMILES: *c1c(F)cc(F)c2nc(N)sc12]

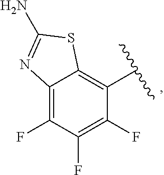 [SMILES: *c1c(F)c(F)c(F)c2nc(N)sc12]

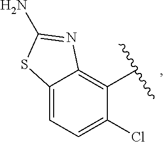 [SMILES: *c1c(Cl)ccc2sc(N)nc12]

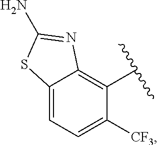 [SMILES: *c1c(C(F)(F)F)ccc2sc(N)nc12]

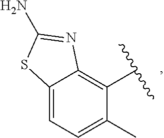 [SMILES: *c1c(C)ccc2sc(N)nc12]

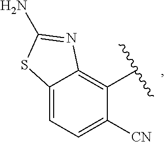 [SMILES: *c1c(C#N)ccc2sc(N)nc12]

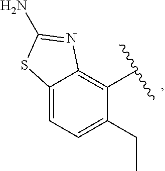 [SMILES: *c1c(CC)ccc2sc(N)nc12]

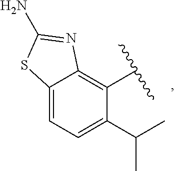 [SMILES: *c1c(C(C)C)ccc2sc(N)nc12]

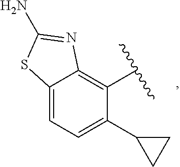 [SMILES: *c1c(C2CC2)ccc2sc(N)nc12]

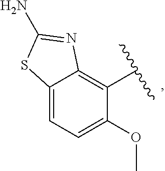 [SMILES: *c1c(OC)ccc2sc(N)nc12]

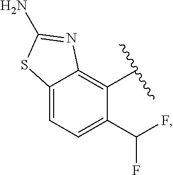 [SMILES: *c1c(C(F)F)ccc2sc(N)nc12]

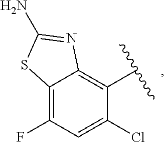 [SMILES: *c1c(Cl)cc(F)c2sc(N)nc12]

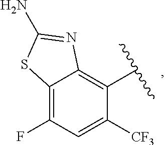 [SMILES: *c1c(C(F)(F)F)cc(F)c2sc(N)nc12]

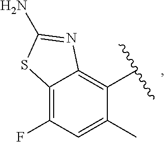 [SMILES: *c1c(C)cc(F)c2sc(N)nc12]

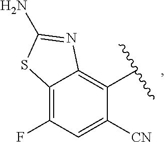 [SMILES: *c1c(C#N)cc(F)c2sc(N)nc12]

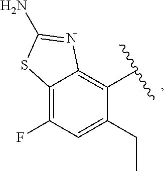 [SMILES: *c1c(CC)cc(F)c2sc(N)nc12]

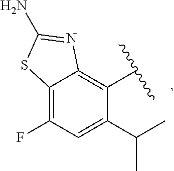 [SMILES: *c1c(C(C)C)cc(F)c2sc(N)nc12]

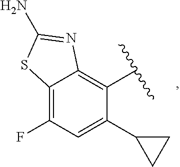 [SMILES: *c1c(C2CC2)cc(F)c2sc(N)nc12]

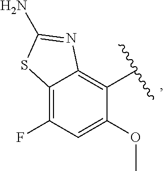 [SMILES: *c1c(OC)cc(F)c2sc(N)nc12]

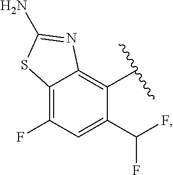 [SMILES: *c1c(C(F)F)cc(F)c2sc(N)nc12]

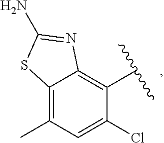 [SMILES: *c1c(Cl)cc(C)c2sc(N)nc12]

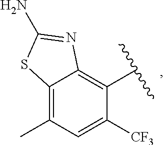 [SMILES: *c1c(C(F)(F)F)cc(C)c2sc(N)nc12]

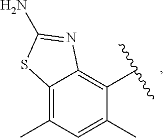

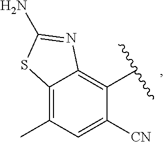 [SMILES: *c1c(C#N)cc(C)c2sc(N)nc12]

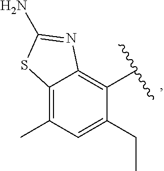 [SMILES: *c1c(CC)cc(C)c2sc(N)nc12]

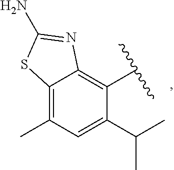 [SMILES: *c1c(C(C)C)cc(C)c2sc(N)nc12]

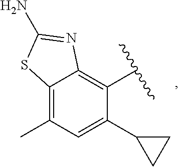 [SMILES: *c1c(C2CC2)cc(C)c2sc(N)nc12]

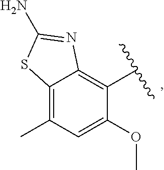

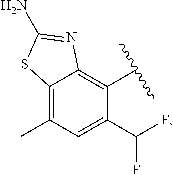 [SMILES: *c1c(C(F)F)cc(C)c2sc(N)nc12]

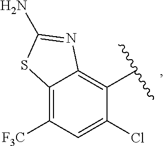 [SMILES: *c1c(Cl)cc(C(F)(F)F)c2sc(N)nc12]

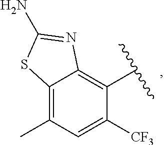 [SMILES: *c1c(C(F)(F)F)cc(C)c2sc(N)nc12]

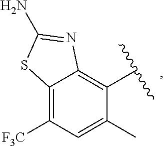 [SMILES: *c1c(C)cc(C(F)(F)F)c2sc(N)nc12]

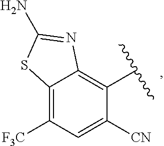 [SMILES: *c1c(C#N)cc(C(F)(F)F)c2sc(N)nc12]

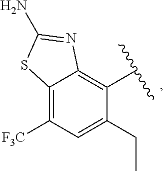 [SMILES: *c1c(CC)cc(C(F)(F)F)c2sc(N)nc12]

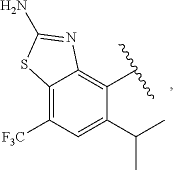 [SMILES: *c1c(C(C)C)cc(C(F)(F)F)c2sc(N)nc12]

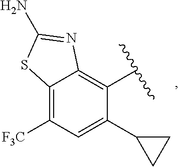 [SMILES: *c1c(C2CC2)cc(C(F)(F)F)c2sc(N)nc12]

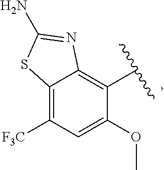 [SMILES: *c1c(OC)cc(C(F)(F)F)c2sc(N)nc12]

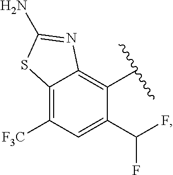 [SMILES: *c1c(C(F)F)cc(C(F)(F)F)c2sc(N)nc12]

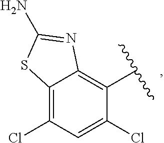 [SMILES: *c1c(Cl)cc(Cl)c2sc(N)nc12]

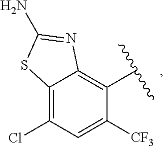 [SMILES: *c1c(C(F)(F)F)cc(Cl)c2sc(N)nc12]

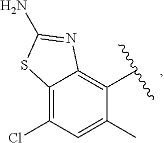 [SMILES: *c1c(C)cc(Cl)c2sc(N)nc12]

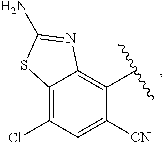 [SMILES: *c1c(C#N)cc(Cl)c2sc(N)nc12]

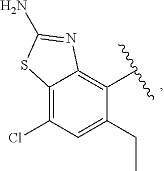 [SMILES: *c1c(CC)cc(Cl)c2sc(N)nc12]

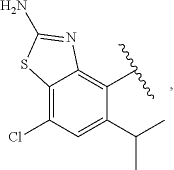 [SMILES: *c1c(C(C)C)cc(Cl)c2sc(N)nc12]

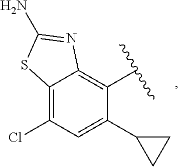 [SMILES: *c1c(C2CC2)cc(Cl)c2sc(N)nc12]

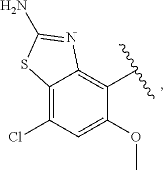 [SMILES: *c1c(OC)cc(Cl)c2sc(N)nc12]

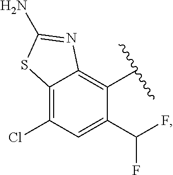 [SMILES: *c1c(C(F)F)cc(Cl)c2sc(N)nc12]

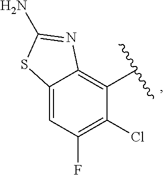 [SMILES: *c1c(Cl)c(F)cc2sc(N)nc12]

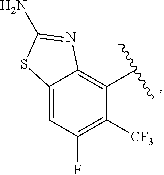 [SMILES: *c1c(C(F)(F)F)c(F)cc2sc(N)nc12]

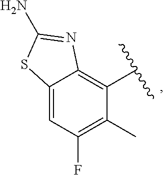 [SMILES: *c1c(C)c(F)cc2sc(N)nc12]

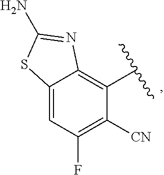 [SMILES: *c1c(C#N)c(F)cc2sc(N)nc12]

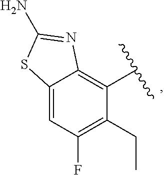 [SMILES: *c1c(CC)c(F)cc2sc(N)nc12]

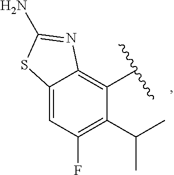 [SMILES: *c1c(C(C)C)c(F)cc2sc(N)nc12]

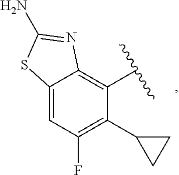 [SMILES: *c1c(C2CC2)c(F)cc2sc(N)nc12]

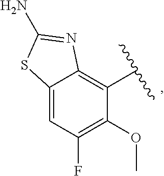 [SMILES: *c1c(OC)c(F)cc2sc(N)nc12]

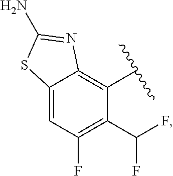 [SMILES: *c1c(C(F)F)c(F)cc2sc(N)nc12]

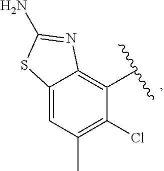 [SMILES: *c1c(Cl)c(C)cc2sc(N)nc12]

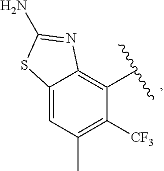 [SMILES: *c1c(C(F)(F)F)c(C)cc2sc(N)nc12]

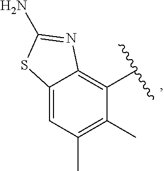 [SMILES: *c1c(C)c(C)cc2sc(N)nc12]

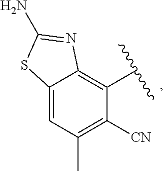 [SMILES: *c1c(C#N)c(C)cc2sc(N)nc12]

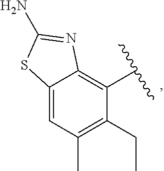 [SMILES: *c1c(CC)c(C)cc2sc(N)nc12]

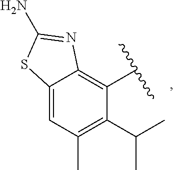 [SMILES: *c1c(C(C)C)c(C)cc2sc(N)nc12]

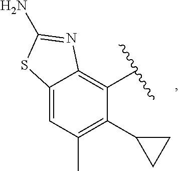 [SMILES: *c1c(C2CC2)c(C)cc2sc(N)nc12]

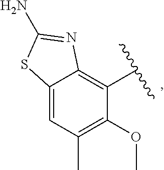 [SMILES: *c1c(OC)c(C)cc2sc(N)nc12]

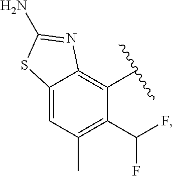 [SMILES: *c1c(C(F)F)c(C)cc2sc(N)nc12]

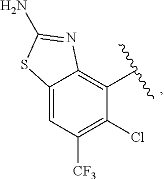 [SMILES: *c1c(Cl)c(C(F)(F)F)cc2sc(N)nc12]

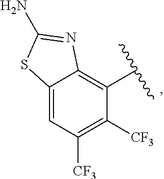 [SMILES: *c1c(C(F)(F)F)c(C(F)(F)F)cc2sc(N)nc12]

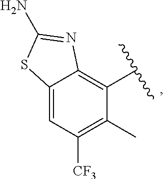 [SMILES: *c1c(C)c(C(F)(F)F)cc2sc(N)nc12]

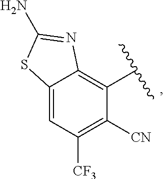 [SMILES: *c1c(C#N)c(C(F)(F)F)cc2sc(N)nc12]

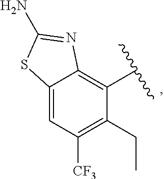 [SMILES: *c1c(CC)c(C(F)(F)F)cc2sc(N)nc12]

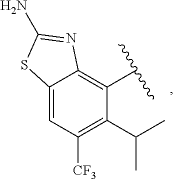 [SMILES: *c1c(C(C)C)c(C(F)(F)F)cc2sc(N)nc12]

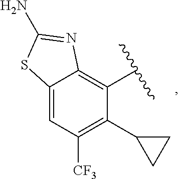 [SMILES: *c1c(C2CC2)c(C(F)(F)F)cc2sc(N)nc12]

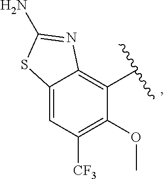 [SMILES: *c1c(OC)c(C(F)(F)F)cc2sc(N)nc12]

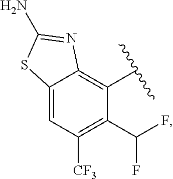 [SMILES: *c1c(C(F)F)c(C(F)(F)F)cc2sc(N)nc12]

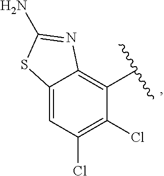 [SMILES: *c1c(Cl)c(Cl)cc2sc(N)nc12]

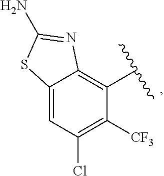 [SMILES: *c1c(C(F)(F)F)c(Cl)cc2sc(N)nc12]

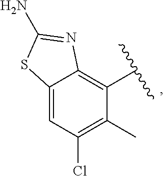 [SMILES: *c1c(C)c(Cl)cc2sc(N)nc12]

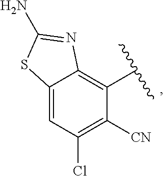 [SMILES: *c1c(C#N)c(Cl)cc2sc(N)nc12]

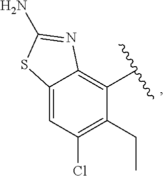 [SMILES: *c1c(CC)c(Cl)cc2sc(N)nc12]

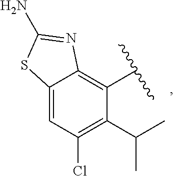 [SMILES: *c1c(C(C)C)c(Cl)cc2sc(N)nc12]

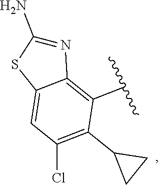 [SMILES: *c1c(C2CC2)c(Cl)cc2sc(N)nc12]

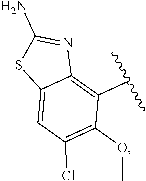 [SMILES: *c1c(OC)c(Cl)cc2sc(N)nc12]

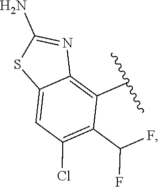 [SMILES: *c1c(C(F)F)c(Cl)cc2sc(N)nc12]

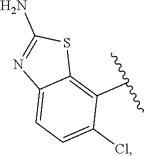 [SMILES: *c1c(Cl)ccc2nc(N)sc12]

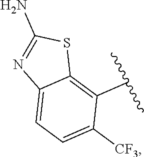 [SMILES: *c1c(C(F)(F)F)ccc2nc(N)sc12]

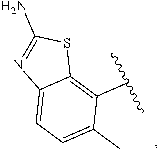 [SMILES: *c1c(C)ccc2nc(N)sc12]

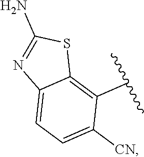 [SMILES: *c1c(C#N)ccc2nc(N)sc12]

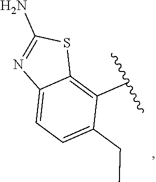 [SMILES: *c1c(CC)ccc2nc(N)sc12]

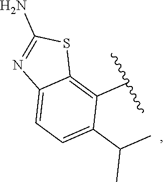 [SMILES: *c1c(C(C)C)ccc2nc(N)sc12]

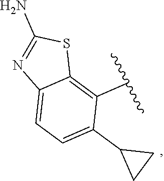 [SMILES: *c1c(C2CC2)ccc2nc(N)sc12]

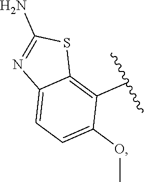 [SMILES: *c1c(OC)ccc2nc(N)sc12]

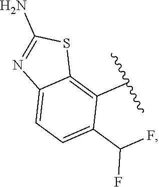 [SMILES: *c1c(C(F)F)ccc2nc(N)sc12]

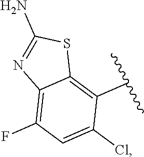 [SMILES: *c1c(Cl)cc(F)c2nc(N)sc12]

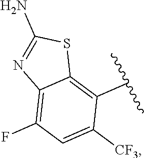 [SMILES: *c1c(C(F)(F)F)cc(F)c2nc(N)sc12]

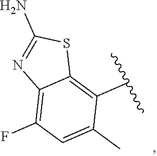 [SMILES: *c1c(C)cc(F)c2nc(N)sc12]

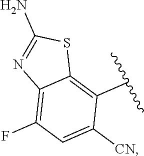 [SMILES: *c1c(C#N)cc(F)c2nc(N)sc12]

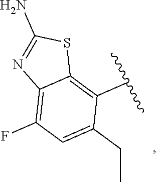 [SMILES: *c1c(CC)cc(F)c2nc(N)sc12]

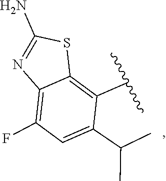 [SMILES: *c1c(C(C)C)cc(F)c2nc(N)sc12]

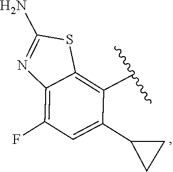 [SMILES: *c1c(C2CC2)cc(F)c2nc(N)sc12]

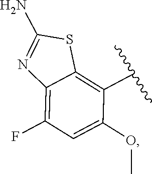 [SMILES: *c1c(OC)cc(F)c2nc(N)sc12]

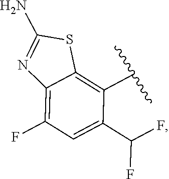 [SMILES: *c1c(C(F)F)cc(F)c2nc(N)sc12]

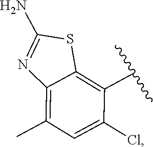 [SMILES: *c1c(Cl)cc(C)c2nc(N)sc12]

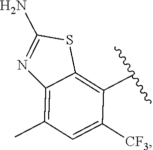 [SMILES: *c1c(C(F)(F)F)cc(C)c2nc(N)sc12]

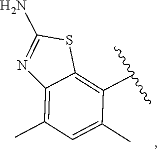 [SMILES: *c1c(C)cc(C)c2nc(N)sc12]

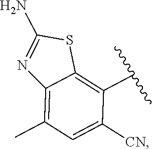 [SMILES: *c1c(C#N)cc(C)c2nc(N)sc12]

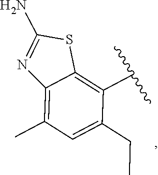 [SMILES: *c1c(CC)cc(C)c2nc(N)sc12]

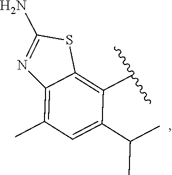 [SMILES: *c1c(C(C)C)cc(C)c2nc(N)sc12]

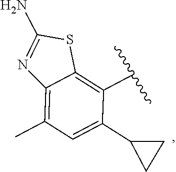 [SMILES: *c1c(C2CC2)cc(C)c2nc(N)sc12]

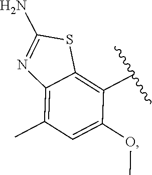 [SMILES: *c1c(OC)cc(C)c2nc(N)sc12]

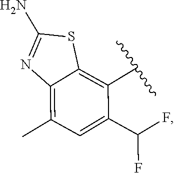 [SMILES: *c1c(C(F)F)cc(C)c2nc(N)sc12]

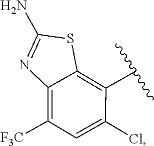 [SMILES: *c1c(Cl)cc(C(F)(F)F)c2nc(N)sc12]

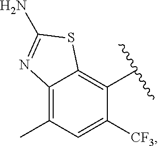 [SMILES: *c1c(C(F)(F)F)cc(C)c2nc(N)sc12]

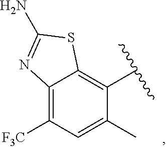 [SMILES: *c1c(C)cc(C(F)(F)F)c2nc(N)sc12]

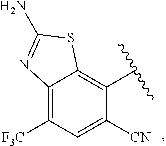 [SMILES: *c1c(C#N)cc(C(F)(F)F)c2nc(N)sc12]

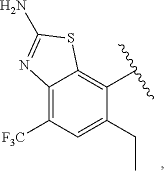 [SMILES: *c1c(CC)cc(C(F)(F)F)c2nc(N)sc12]

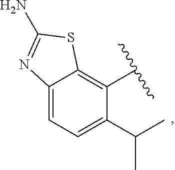 [SMILES: *c1c(C(C)C)ccc2nc(N)sc12]

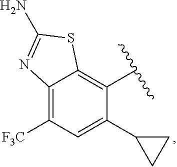 [SMILES: *c1c(C2CC2)cc(C(F)(F)F)c2nc(N)sc12]

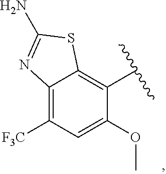 [SMILES: *c1c(OC)cc(C(F)(F)F)c2nc(N)sc12]

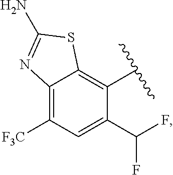 [SMILES: *c1c(C(F)F)cc(C(F)(F)F)c2nc(N)sc12]

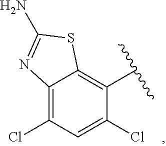 [SMILES: *c1c(Cl)cc(Cl)c2nc(N)sc12]

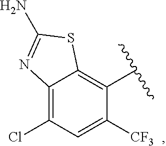 [SMILES: *c1c(C(F)(F)F)cc(Cl)c2nc(N)sc12]

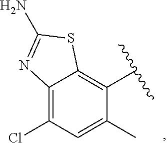 [SMILES: *c1c(C)cc(Cl)c2nc(N)sc12]

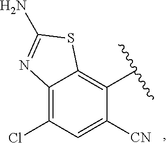 [SMILES: *c1c(C#N)cc(Cl)c2nc(N)sc12]

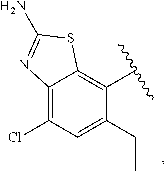 [SMILES: *c1c(CC)cc(Cl)c2nc(N)sc12]

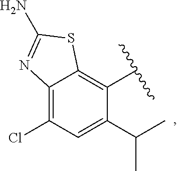 [SMILES: *c1c(C(C)C)cc(Cl)c2nc(N)sc12]

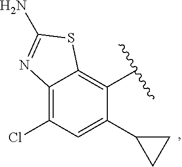 [SMILES: *c1c(C2CC2)cc(Cl)c2nc(N)sc12]

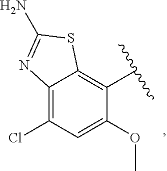 [SMILES: *c1c(OC)cc(Cl)c2nc(N)sc12]

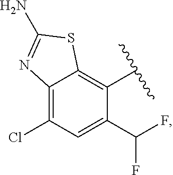 [SMILES: *c1c(C(F)F)cc(Cl)c2nc(N)sc12]

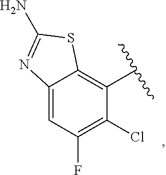 [SMILES: *c1c(Cl)c(F)cc2nc(N)sc12]

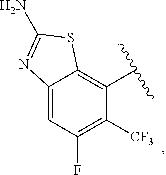 [SMILES: *c1c(C(F)(F)F)c(F)cc2nc(N)sc12]

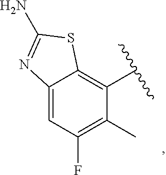 [SMILES: *c1c(C)c(F)cc2nc(N)sc12]

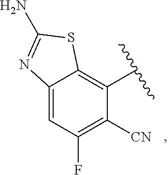 [SMILES: *c1c(C#N)c(F)cc2nc(N)sc12]

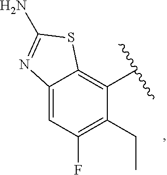 [SMILES: *c1c(CC)c(F)cc2nc(N)sc12]

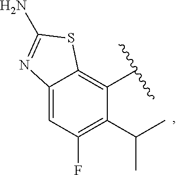 [SMILES: *c1c(C(C)C)c(F)cc2nc(N)sc12]

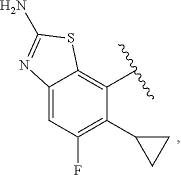 [SMILES: *c1c(C2CC2)c(F)cc2nc(N)sc12]

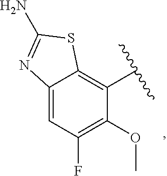 [SMILES: *c1c(OC)c(F)cc2nc(N)sc12]

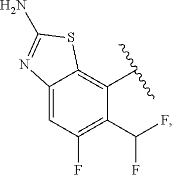 [SMILES: *c1c(C(F)F)c(F)cc2nc(N)sc12]

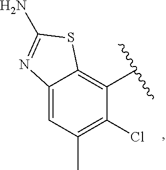 [SMILES: *c1c(Cl)c(C)cc2nc(N)sc12]

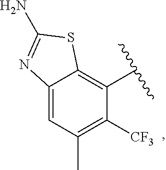 [SMILES: *c1c(C(F)(F)F)c(C)cc2nc(N)sc12]

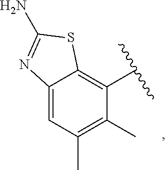 [SMILES: *c1c(C)c(C)cc2nc(N)sc12]

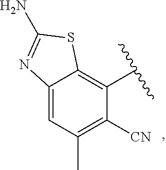 [SMILES: *c1c(C#N)c(C)cc2nc(N)sc12]

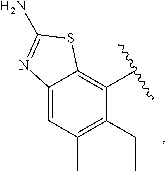 [SMILES: *c1c(CC)c(C)cc2nc(N)sc12]

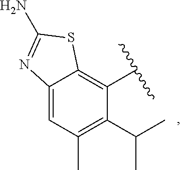 [SMILES: *c1c(C(C)C)c(C)cc2nc(N)sc12]

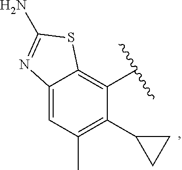 [SMILES: *c1c(C2CC2)c(C)cc2nc(N)sc12]

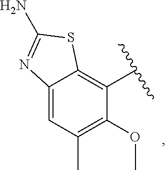 [SMILES: *c1c(OC)c(C)cc2nc(N)sc12]

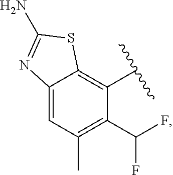 [SMILES: *c1c(C(F)F)c(C)cc2nc(N)sc12]

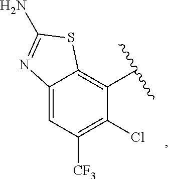 [SMILES: *c1c(Cl)c(C(F)(F)F)cc2nc(N)sc12]

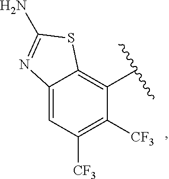 [SMILES: *c1c(C(F)(F)F)c(C(F)(F)F)cc2nc(N)sc12]

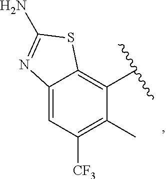 [SMILES: *c1c(C)c(C(F)(F)F)cc2nc(N)sc12]

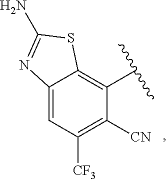 [SMILES: *c1c(C#N)c(C(F)(F)F)cc2nc(N)sc12]

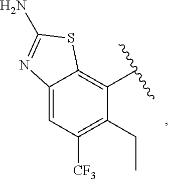 [SMILES: *c1c(CC)c(C(F)(F)F)cc2nc(N)sc12]

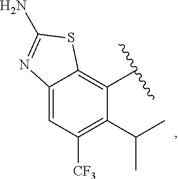 [SMILES: *c1c(C(C)C)c(C(F)(F)F)cc2nc(N)sc12]

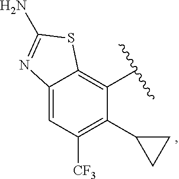 [SMILES: *c1c(C2CC2)c(C(F)(F)F)cc2nc(N)sc12]

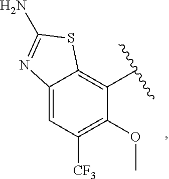 [SMILES: *c1c(OC)c(C(F)(F)F)cc2nc(N)sc12]

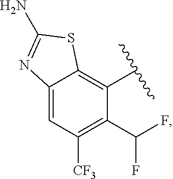 [SMILES: *c1c(C(F)F)c(C(F)(F)F)cc2nc(N)sc12]

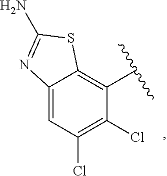 [SMILES: *c1c(Cl)c(Cl)cc2nc(N)sc12]

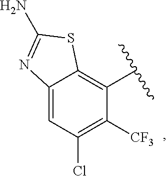 [SMILES: *c1c(C(F)(F)F)c(Cl)cc2nc(N)sc12]

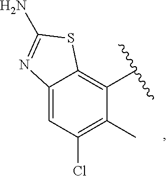 [SMILES: *c1c(C)c(Cl)cc2nc(N)sc12]

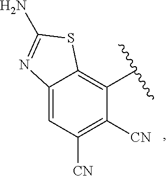 [SMILES: *c1c(C#N)c(C#N)cc2nc(N)sc12]

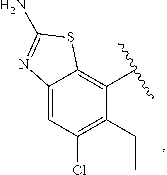 [SMILES: *c1c(CC)c(Cl)cc2nc(N)sc12]

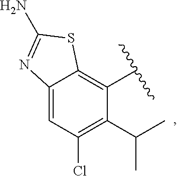 [SMILES: *c1c(C(C)C)c(Cl)cc2nc(N)sc12]

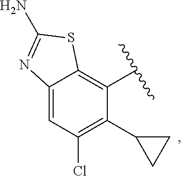 [SMILES: *c1c(C2CC2)c(Cl)cc2nc(N)sc12]

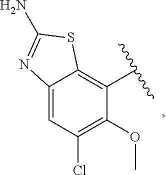 [SMILES: *c1c(OC)c(Cl)cc2nc(N)sc12]

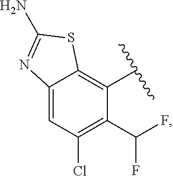 [SMILES: *c1c(C(F)F)c(Cl)cc2nc(N)sc12]

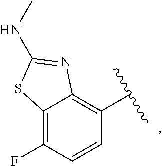 [SMILES: *c1ccc(F)c2sc(NC)nc12]

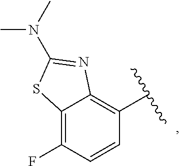 [SMILES: *c1ccc(F)c2sc(N(C)C)nc12]

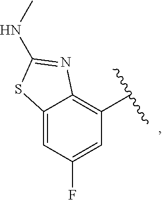 [SMILES: *c1cc(F)cc2sc(NC)nc12]

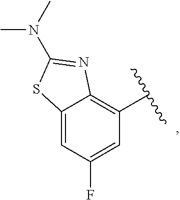 [SMILES: *c1cc(F)cc2sc(N(C)C)nc12]

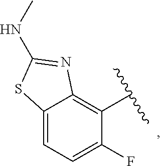 [SMILES: *c1c(F)ccc2sc(NC)nc12]

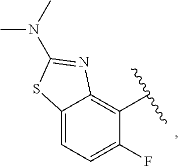 [SMILES: *c1c(F)ccc2sc(N(C)C)nc12]

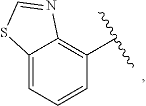 [SMILES: *c1cccc2scnc12]

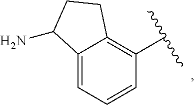 [SMILES: *c1cccc2c1CCC2N]

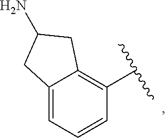 [SMILES: *c1cccc2c1CC(N)C2]

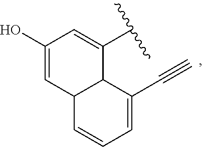 [SMILES: *C1=CC(O)=CC2C=CC=C(C#C)C12]

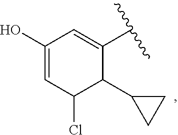 [SMILES: *C1=CC(O)=CC(Cl)C1C1CC1]

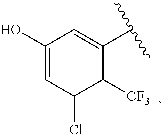 [SMILES: *C1=CC(O)=CC(Cl)C1C(F)(F)F]

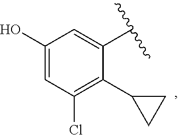 [SMILES: *c1cc(O)cc(Cl)c1C1CC1]

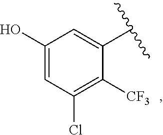 [SMILES: *c1cc(O)cc(Cl)c1C(F)(F)F]

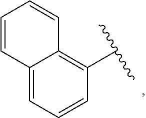 [SMILES: *c1cccc2ccccc12]

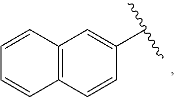 [SMILES: *c1ccc2ccccc2c1]

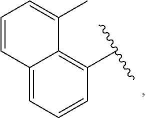 [SMILES: *c1cccc2cccc(C)c12]

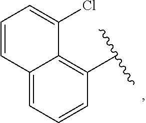 [SMILES: *c1cccc2cccc(Cl)c12]

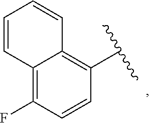 [SMILES: *c1ccc(F)c2ccccc12]

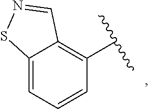 [SMILES: *c1cccc2sncc12]

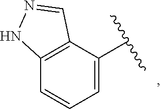 [SMILES: *c1cccc2[nH]ncc12]

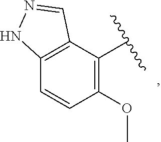 [SMILES: *c1c(OC)ccc2[nH]ncc12]

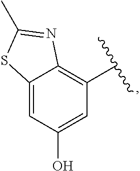 [SMILES: *c1cc(O)cc2sc(C)nc12]

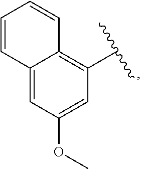 [SMILES: *c1cc(OC)cc2ccccc12]

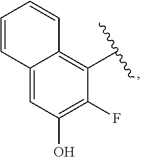 [SMILES: *c1c(F)c(O)cc2ccccc12]

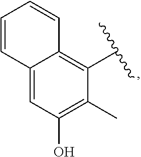 [SMILES: *c1c(C)c(O)cc2ccccc12]

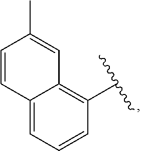 [SMILES: *c1cccc2ccc(C)cc12]

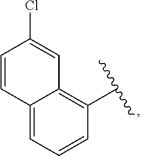 [SMILES: *c1cccc2ccc(Cl)cc12]

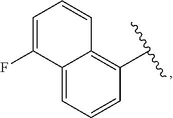 [SMILES: *c1cccc2c(F)cccc12]

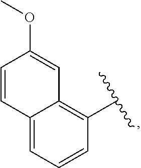 [SMILES: *c1cccc2ccc(OC)cc12]

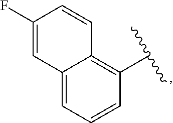 [SMILES: *c1cccc2cc(F)ccc12]

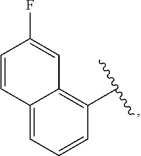 [SMILES: *c1cccc2ccc(F)cc12]

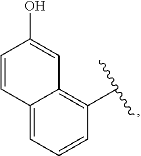 [SMILES: *c1cccc2ccc(O)cc12]

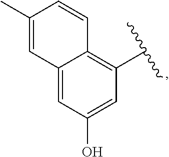 [SMILES: *c1cc(O)cc2cc(C)ccc12]

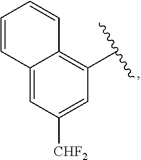 [SMILES: *c1cc(C(F)F)cc2ccccc12]

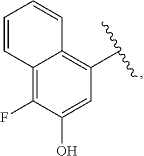 [SMILES: *c1cc(O)c(F)c2ccccc12]

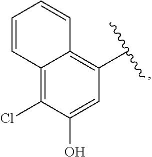 [SMILES: *c1cc(O)c(Cl)c2ccccc12]

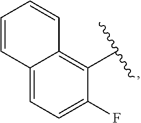 [SMILES: *c1c(F)ccc2ccccc12]

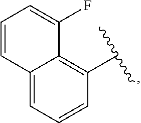 [SMILES: *c1cccc2cccc(F)c12]

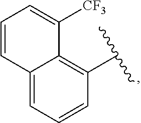 [SMILES: *c1cccc2cccc(C(F)(F)F)c12]

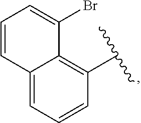 [SMILES: *c1cccc2cccc(Br)c12]

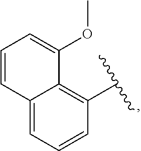 [SMILES: *c1cccc2cccc(OC)c12]

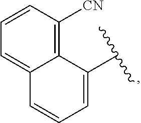 [SMILES: *c1cccc2cccc(C#N)c12]

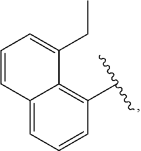 [SMILES: *c1cccc2cccc(CC)c12]

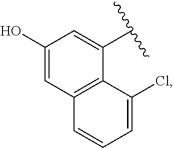 [SMILES: *c1cc(O)cc2cccc(Cl)c12]

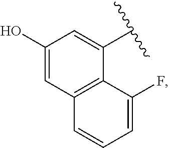 [SMILES: *c1cc(O)cc2cccc(F)c12]

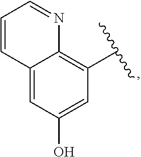 [SMILES: *c1cc(O)cc2cccnc12]

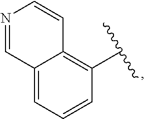 [SMILES: *c1cccc2cnccc12]

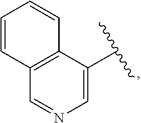 [SMILES: *c1cncc2ccccc12]

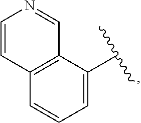 [SMILES: *c1cccc2ccncc12]

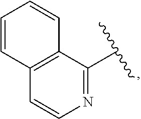 [SMILES: *c1nccc2ccccc12]

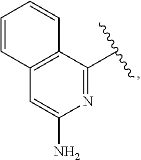 [SMILES: *c1nc(N)cc2ccccc12]

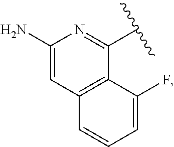 [SMILES: *c1nc(N)cc2cccc(F)c12]

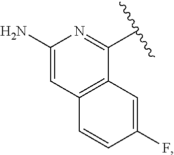 [SMILES: *c1nc(N)cc2ccc(F)cc12]

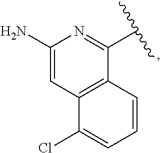 [SMILES: *c1nc(N)cc2c(Cl)cccc12]

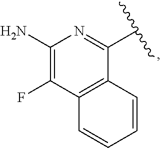 [SMILES: *c1nc(N)c(F)c2ccccc12]

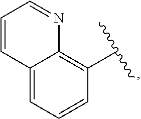 [SMILES: *c1cccc2cccnc12]

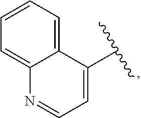 [SMILES: *c1ccnc2ccccc12]

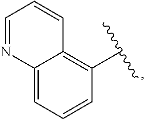 [SMILES: *c1cccc2ncccc12]

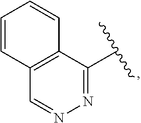 [SMILES: *c1nncc2ccccc12]

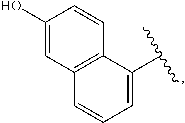 [SMILES: *c1cccc2cc(O)ccc12]

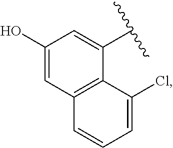 [SMILES: *c1cc(O)cc2cccc(Cl)c12]

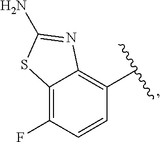 [SMILES: *c1ccc(F)c2sc(N)nc12]

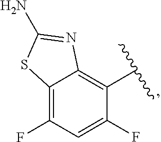 [SMILES: *c1c(F)cc(F)c2sc(N)nc12]

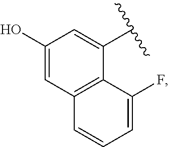 [SMILES: *c1cc(O)cc2cccc(F)c12]

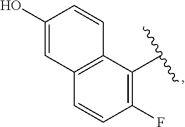 [SMILES: *c1c(F)ccc2cc(O)ccc12]

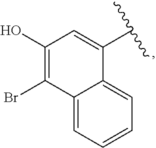 [SMILES: *c1cc(O)c(Br)c2ccccc12]

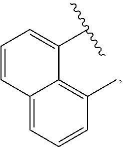 [SMILES: *c1cccc2cccc(C)c12]

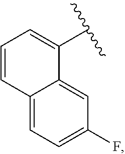 [SMILES: *c1cccc2ccc(F)cc12]

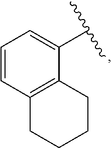 [SMILES: *c1cccc2c1CCCC2]

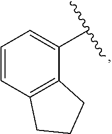 [SMILES: *c1cccc2c1CCC2]

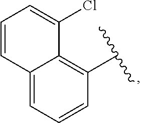 [SMILES: *c1cccc2cccc(Cl)c12]

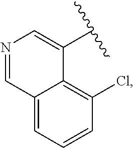 [SMILES: *c1cncc2cccc(Cl)c12]

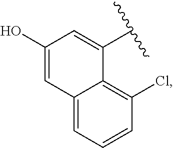 [SMILES: *c1cc(O)cc2cccc(Cl)c12]

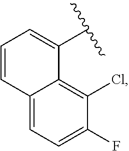 [SMILES: *c1cccc2ccc(F)c(Cl)c12]

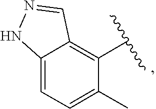 [SMILES: *c1c(C)ccc2[nH]ncc12]

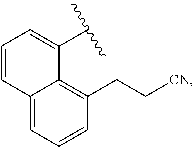 [SMILES: *c1cccc2cccc(CCC#N)c12]

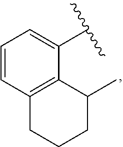 [SMILES: *c1cccc2c1C(C)CCC2]

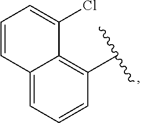 [SMILES: *c1cccc2cccc(Cl)c12]

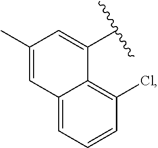 [SMILES: *c1cc(C)cc2cccc(Cl)c12]

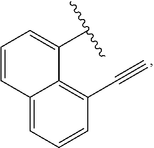 [SMILES: *c1cccc2cccc(C#C)c12]

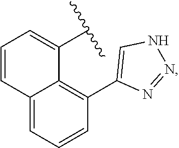 [SMILES: *c1cccc2cccc(-c3c[nH]nn3)c12]

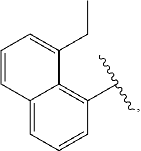 [SMILES: *c1cccc2cccc(CC)c12]

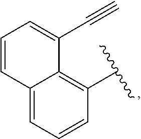 [SMILES: *c1cccc2cccc(C#C)c12]

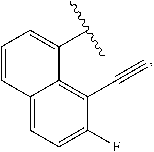 [SMILES: *c1cccc2ccc(F)c(C#C)c12]

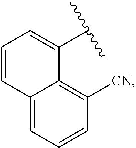 [SMILES: *c1cccc2cccc(C#N)c12]

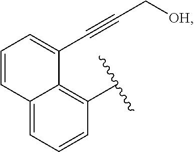 [SMILES: *c1cccc2cccc(C#CCO)c12]

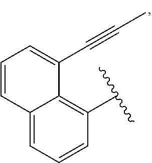 [SMILES: *c1cccc2cccc(C#CC)c12]

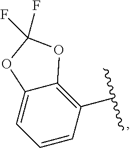 [SMILES: *c1cccc2c1OC(F)(F)O2]

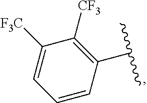 [SMILES: *c1cccc(C(F)(F)F)c1C(F)(F)F]

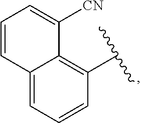 [SMILES: *c1cccc2cccc(C#N)c12]

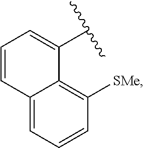 [SMILES: *c1cccc2cccc(SC)c12]

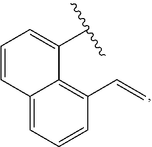 [SMILES: *c1cccc2cccc(C=C)c12]

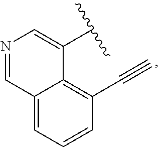 [SMILES: *c1cncc2cccc(C#C)c12]

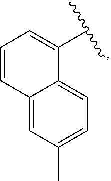 [SMILES: *c1cccc2cc(C)ccc12]

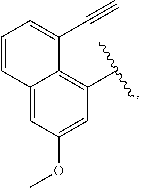 [SMILES: *c1cc(OC)cc2cccc(C#C)c12]

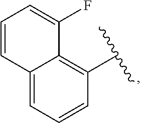 [SMILES: *c1cccc2cccc(F)c12]

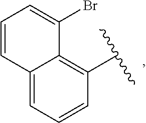 [SMILES: *c1cccc2cccc(Br)c12]

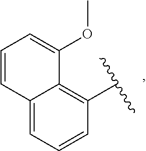 [SMILES: *c1cccc2cccc(OC)c12]

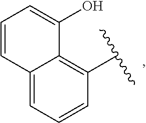 [SMILES: *c1cccc2cccc(O)c12]

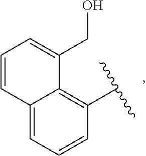 [SMILES: *c1cccc2cccc(CO)c12]

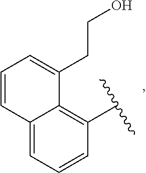 [SMILES: *c1cccc2cccc(CCO)c12]

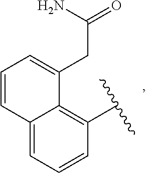 [SMILES: *c1cccc2cccc(CC(N)=O)c12]

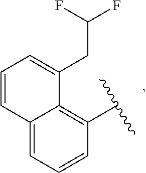 [SMILES: *c1cccc2cccc(CC(F)F)c12]

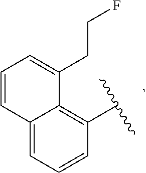 [SMILES: *c1cccc2cccc(CCF)c12]

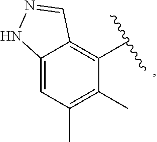 [SMILES: *c1c(C)c(C)cc2[nH]ncc12]

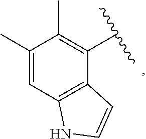 [SMILES: *c1c(C)c(C)cc2[nH]ccc12]

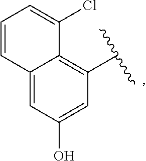 [SMILES: *c1cc(O)cc2cccc(Cl)c12]

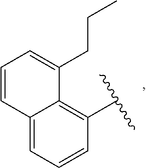 [SMILES: *c1cccc2cccc(CCC)c12]

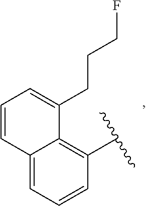 [SMILES: *c1cccc2cccc(CCCF)c12]

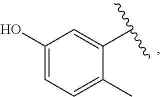 [SMILES: *c1cc(O)ccc1C]

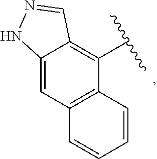 [SMILES: *c1c2ccccc2cc2[nH]ncc12]

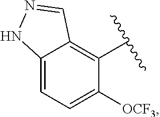 [SMILES: *c1c(OC(F)(F)F)ccc2[nH]ncc12]

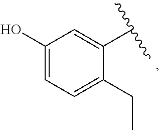 [SMILES: *c1cc(O)ccc1CC]

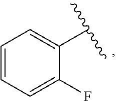 [SMILES: *c1ccccc1F]

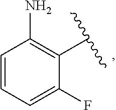 [SMILES: *c1c(N)cccc1F]

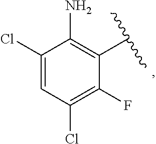 [SMILES: *c1c(N)c(Cl)cc(Cl)c1F]

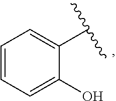 [SMILES: *c1ccccc1O]

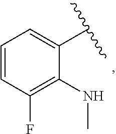 [SMILES: *c1cccc(F)c1NC]

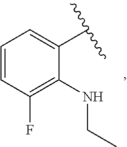 [SMILES: *c1cccc(F)c1NCC]

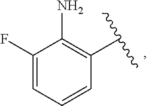 [SMILES: *c1cccc(F)c1N]

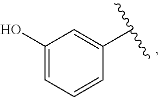 [SMILES: *c1cccc(O)c1]

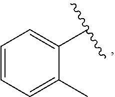 [SMILES: *c1ccccc1C]

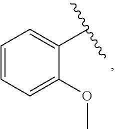 [SMILES: *c1ccccc1OC]

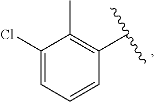 [SMILES: *c1cccc(Cl)c1C]

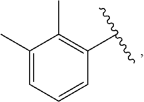 [SMILES: *c1cccc(C)c1C]

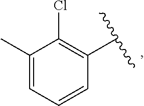 [SMILES: *c1cccc(C)c1Cl]

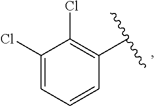 [SMILES: *c1cccc(Cl)c1Cl]

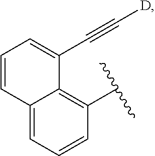

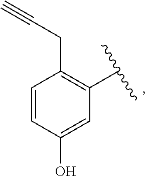 [SMILES: *c1cc(O)ccc1CC#C]

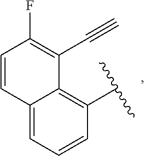 [SMILES: *c1cccc2ccc(F)c(C#C)c12]

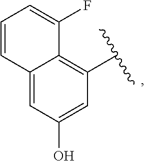 [SMILES: *c1cc(O)cc2cccc(F)c12]

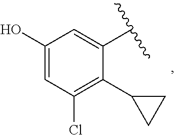 [SMILES: *c1cc(O)cc(Cl)c1C1CC1]

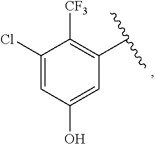 [SMILES: *c1cc(O)cc(Cl)c1C(F)(F)F]

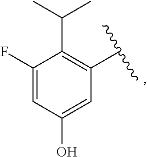 [SMILES: *c1cc(O)cc(F)c1C(C)C]

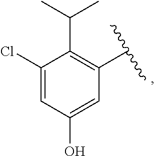 [SMILES: *c1cc(O)cc(Cl)c1C(C)C]

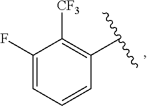 [SMILES: *c1cccc(F)c1C(F)(F)F]

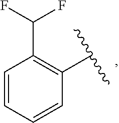 [SMILES: *c1ccccc1C(F)F]

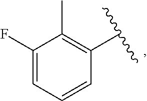 [SMILES: *c1cccc(F)c1C]

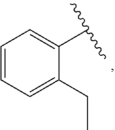 [SMILES: *c1ccccc1CC]

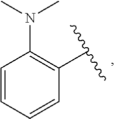 [SMILES: *c1ccccc1N(C)C]

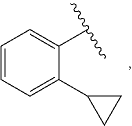 [SMILES: *c1ccccc1C1CC1]

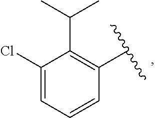 [SMILES: *c1cccc(Cl)c1C(C)C]

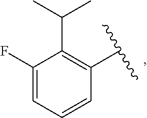 [SMILES: *c1cccc(F)c1C(C)C]

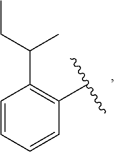 [SMILES: *c1ccccc1C(C)CC]

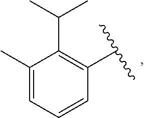 [SMILES: *c1cccc(C)c1C(C)C]

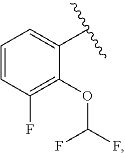 [SMILES: *c1cccc(F)c1OC(F)F]

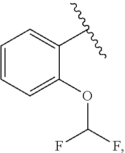 [SMILES: *c1ccccc1OC(F)F]

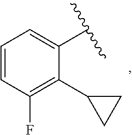 [SMILES: *c1cccc(F)c1C1CC1]

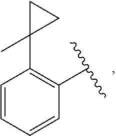 [SMILES: *c1ccccc1C1(C)CC1]

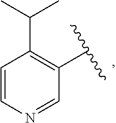 [SMILES: *c1cnccc1C(C)C]

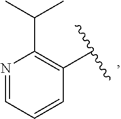 [SMILES: *c1cccnc1C(C)C]

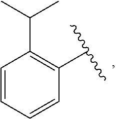 [SMILES: *c1ccccc1C(C)C]

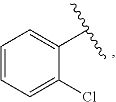 [SMILES: *c1ccccc1Cl]

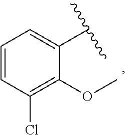 [SMILES: *c1cccc(Cl)c1OC]

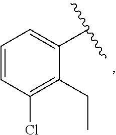 [SMILES: *c1cccc(Cl)c1CC]

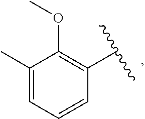 [SMILES: *c1cccc(C)c1OC]

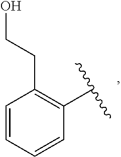 [SMILES: *c1ccccc1CCO]

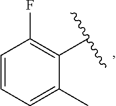 [SMILES: *c1c(C)cccc1F]

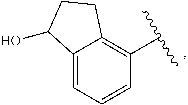 [SMILES: *c1cccc2c1CCC2O]

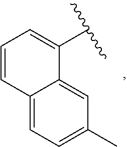 [SMILES: *c1cccc2ccc(C)cc12]

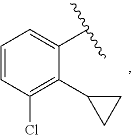 [SMILES: *c1cccc(Cl)c1C1CC1]

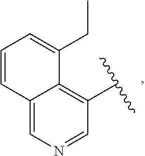 [SMILES: *c1cncc2cccc(CC)c12]

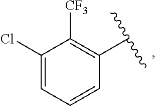 [SMILES: *c1cccc(Cl)c1C(F)(F)F]

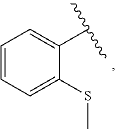 [SMILES: *c1ccccc1SC]

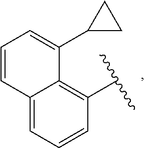 [SMILES: *c1cccc2cccc(C3CC3)c12]

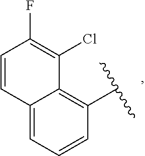 [SMILES: *c1cccc2ccc(F)c(Cl)c12]

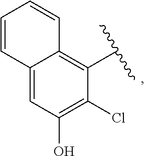 [SMILES: *c1c(Cl)c(O)cc2ccccc12]

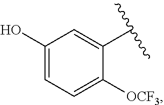 [SMILES: *c1cc(O)ccc1OC(F)(F)F]

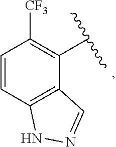 [SMILES: *c1c(C(F)(F)F)ccc2[nH]ncc12]

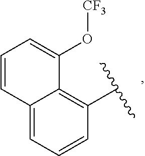 [SMILES: *c1cccc2cccc(OC(F)(F)F)c12]

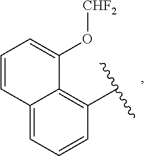 [SMILES: *c1cccc2cccc(OC(F)F)c12]

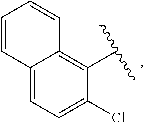 [SMILES: *c1c(Cl)ccc2ccccc12]

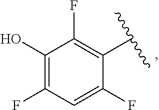 [SMILES: *c1c(F)cc(F)c(O)c1F]

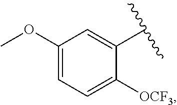 [SMILES: *c1cc(OC)ccc1OC(F)(F)F]

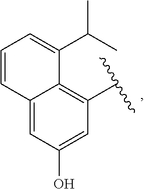 [SMILES: *c1cc(O)cc2cccc(C(C)C)c12]

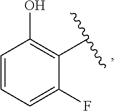 [SMILES: *c1c(O)cccc1F]

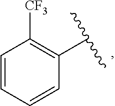 [SMILES: *c1ccccc1C(F)(F)F]

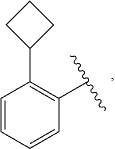 [SMILES: *c1ccccc1C1CCC1]

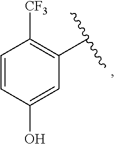 [SMILES: *c1cc(O)ccc1C(F)(F)F]

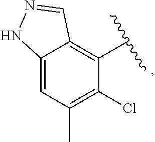 [SMILES: *c1c(Cl)c(C)cc2[nH]ncc12]

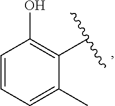 [SMILES: *c1c(C)cccc1O]

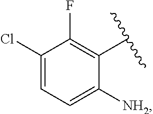 [SMILES: *c1c(N)ccc(Cl)c1F]

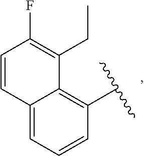 [SMILES: *c1cccc2ccc(F)c(CC)c12]

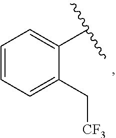 [SMILES: *c1ccccc1CC(F)(F)F]

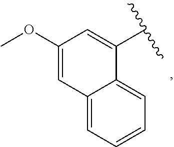 [SMILES: *c1cc(OC)cc2ccccc12]

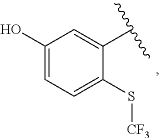 [SMILES: *c1cc(O)ccc1SC(F)(F)F]

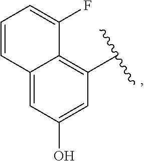 [SMILES: *c1cc(O)cc2cccc(F)c12]

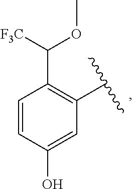 [SMILES: *c1cc(O)ccc1C(OC)C(F)(F)F]

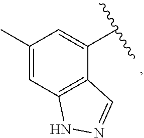 [SMILES: *c1cc(C)cc2[nH]ncc12]

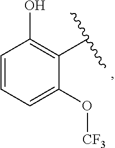 [SMILES: *c1c(O)cccc1OC(F)(F)F]

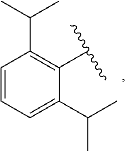 [SMILES: *c1c(C(C)C)cccc1C(C)C]

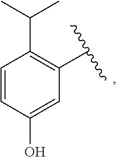 [SMILES: *c1cc(O)ccc1C(C)C]

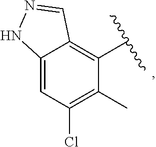 [SMILES: *c1c(C)c(Cl)cc2[nH]ncc12]

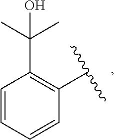 [SMILES: *c1ccccc1C(C)(C)O]

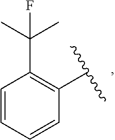 [SMILES: *c1ccccc1C(C)(C)F]

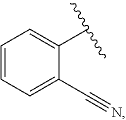 [SMILES: *c1ccccc1C#N]

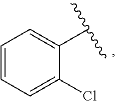 [SMILES: *c1ccccc1Cl]

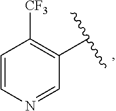 [SMILES: *c1cnccc1C(F)(F)F]

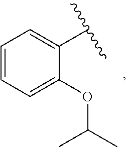 [SMILES: *c1ccccc1OC(C)C]

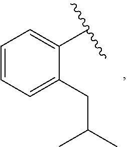 [SMILES: *c1ccccc1CC(C)C]

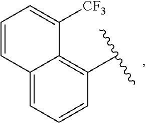 [SMILES: *c1cccc2cccc(C(F)(F)F)c12]

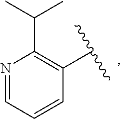 [SMILES: *c1cccnc1C(C)C]

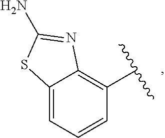 [SMILES: *c1cccc2sc(N)nc12]

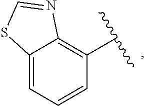 [SMILES: *c1cccc2scnc12]

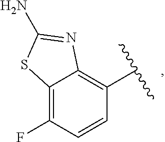 [SMILES: *c1ccc(F)c2sc(N)nc12]

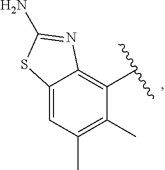 [SMILES: *c1c(C)c(C)cc2sc(N)nc12]

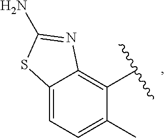 [SMILES: *c1c(C)ccc2sc(N)nc12]

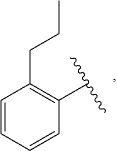 [SMILES: *c1ccccc1CCC]

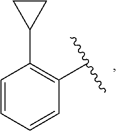 [SMILES: *c1ccccc1C1CC1]

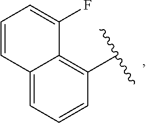 [SMILES: *c1cccc2cccc(F)c12]

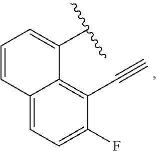 [SMILES: *c1cccc2ccc(F)c(C#C)c12]

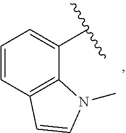 [SMILES: *c1cccc2ccn(C)c12]

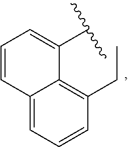 [SMILES: *c1cccc2cccc(CC)c12]

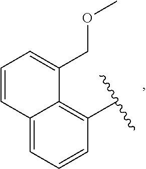 [SMILES: *c1cccc2cccc(COC)c12]

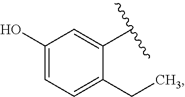 [SMILES: *c1cc(O)ccc1CC]

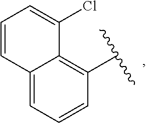 [SMILES: *c1cccc2cccc(Cl)c12]

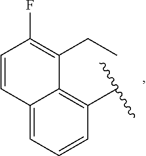 [SMILES: *c1cccc2ccc(F)c(CC)c12]

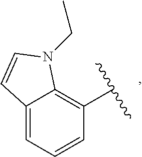 [SMILES: *c1cccc2ccn(CC)c12]

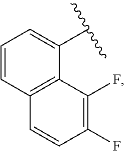 [SMILES: *c1cccc2ccc(F)c(F)c12]

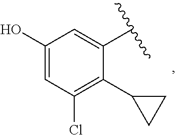 [SMILES: *c1cc(O)cc(Cl)c1C1CC1]

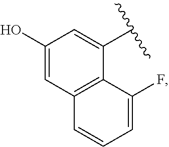 [SMILES: *c1cc(O)cc2cccc(F)c12]

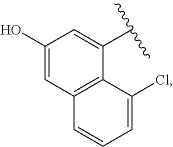 [SMILES: *c1cc(O)cc2cccc(Cl)c12]

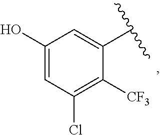 [SMILES: *c1cc(O)cc(Cl)c1C(F)(F)F]

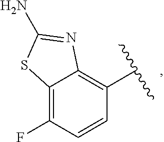 [SMILES: *c1ccc(F)c2sc(N)nc12]

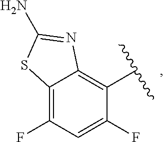 [SMILES: *c1c(F)cc(F)c2sc(N)nc12]

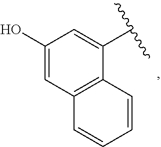 [SMILES: *c1cc(O)cc2ccccc12]

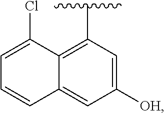 [SMILES: *c1cc(O)cc2cccc(Cl)c12]

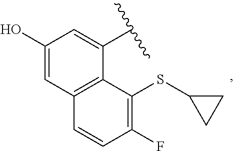 [SMILES: *c1cc(O)cc2ccc(F)c(SC3CC3)c12]

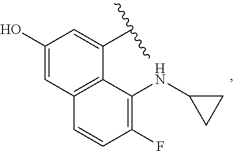 [SMILES: *c1cc(O)cc2ccc(F)c(NC3CC3)c12]

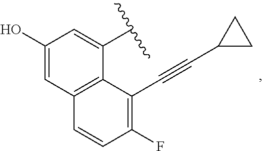 [SMILES: *c1cc(O)cc2ccc(F)c(C#CC3CC3)c12]

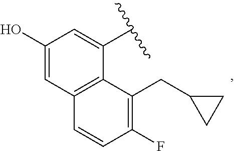 [SMILES: *c1cc(O)cc2ccc(F)c(CC3CC3)c12]

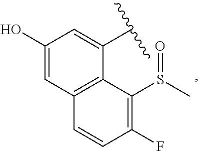 [SMILES: *c1cc(O)cc2ccc(F)c(S(C)=O)c12]

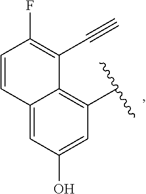 [SMILES: *c1cc(O)cc2ccc(F)c(C#C)c12]

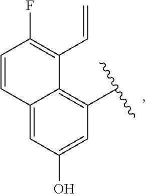 [SMILES: *c1cc(O)cc2ccc(F)c(C=C)c12]

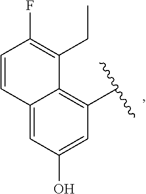 [SMILES: *c1cc(O)cc2ccc(F)c(CC)c12]

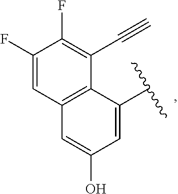 [SMILES: *c1cc(O)cc2cc(F)c(F)c(C#C)c12]

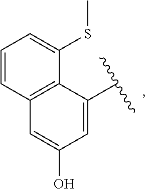 [SMILES: *c1cc(O)cc2cccc(SC)c12]

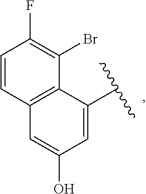 [SMILES: *c1cc(O)cc2ccc(F)c(Br)c12]

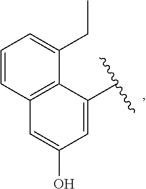 [SMILES: *c1cc(O)cc2cccc(CC)c12]

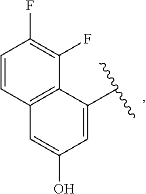 [SMILES: *c1cc(O)cc2ccc(F)c(F)c12]

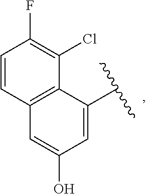 [SMILES: *c1cc(O)cc2ccc(F)c(Cl)c12]

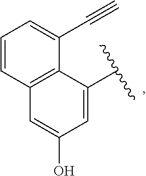 [SMILES: *c1cc(O)cc2cccc(C#C)c12]

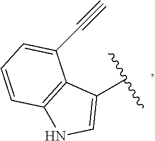 [SMILES: *c1c[nH]c2cccc(C#C)c12]

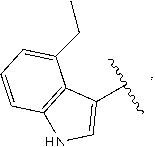 [SMILES: *c1c[nH]c2cccc(CC)c12]

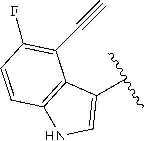 [SMILES: *c1c[nH]c2ccc(F)c(C#C)c12]

or

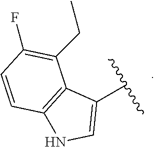 [SMILES: *c1c[nH]c2ccc(F)c(CC)c12]

[26]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [25], wherein, R₄ is selected from any moiety in the Table 10:

TABLE 10

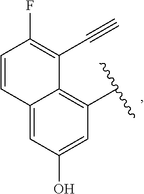 [SMILES: *c1cc(O)cc2ccc(F)c(C#C)c12]

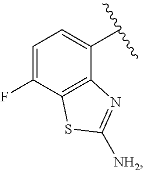 [SMILES: *c1ccc(F)c2sc(N)nc12]

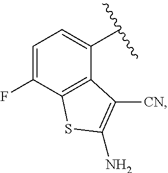 [SMILES: *c1ccc(F)c2sc(N)c(C#N)c12]

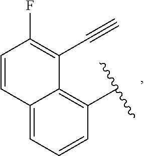 [SMILES: *c1cccc2ccc(F)c(C#C)c12]

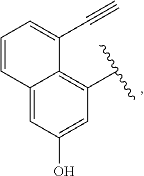 [SMILES: *c1cc(O)cc2cccc(C#C)c12]

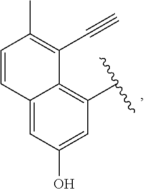 [SMILES: *c1cc(O)cc2ccc(C)c(C#C)c12]

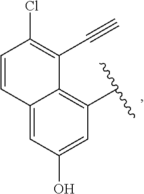 [SMILES: *c1cc(O)cc2ccc(Cl)c(C#C)c12]

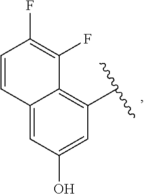 [SMILES: *c1cc(O)cc2ccc(F)c(F)c12]

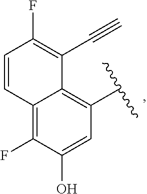 [SMILES: *c1cc(O)c(F)c2ccc(F)c(C#C)c12]

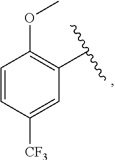 [SMILES: *c1cc(C(F)(F)F)ccc1OC]

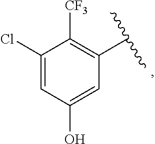 [SMILES: *c1cc(O)cc(Cl)c1C(F)(F)F]

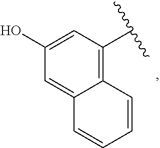 [SMILES: *c1cc(O)cc2ccccc12]

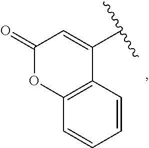 [SMILES: *c1cc(=O)oc2ccccc12]

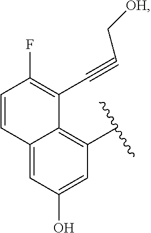 [SMILES: *c1cc(O)cc2ccc(F)c(C#CCO)c12]

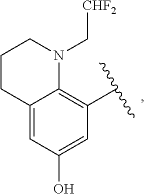 [SMILES: *c1cc(O)cc2c1N(CC(F)F)CCC2]

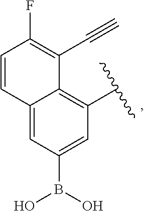 [SMILES: *c1cc(B(O)O)cc2ccc(F)c(C#C)c12]

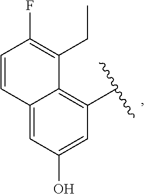 [SMILES: *c1cc(O)cc2ccc(F)c(CC)c12]

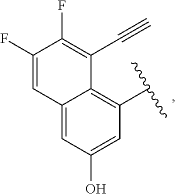 [SMILES: *c1cc(O)cc2cc(F)c(F)c(C#C)c12]

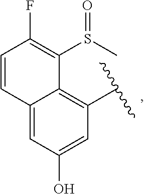 [SMILES: *c1cc(O)cc2ccc(F)c(S(C)=O)c12]

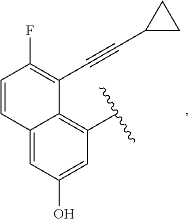 [SMILES: *c1cc(O)cc2ccc(F)c(C#CC3CC3)c12]

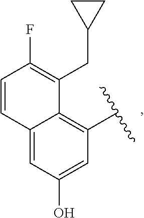 [SMILES: *c1cc(O)cc2ccc(F)c(CC3CC3)c12]

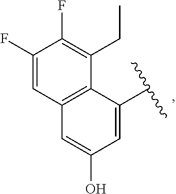 [SMILES: *c1cc(O)cc2cc(F)c(F)c(CC)c12]

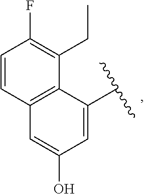 [SMILES: *c1cc(O)cc2ccc(F)c(CC)c12]

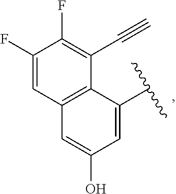 [SMILES: *c1cc(O)cc2cc(F)c(F)c(C#C)c12]

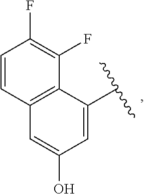 [SMILES: *c1cc(O)cc2ccc(F)c(F)c12]

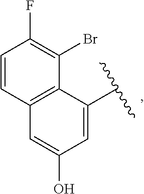 [SMILES: *c1cc(O)cc2ccc(F)c(Br)c12]

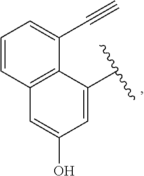 [SMILES: *c1cc(O)cc2cccc(C#C)c12]

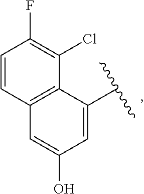 [SMILES: *c1cc(O)cc2ccc(F)c(Cl)c12]

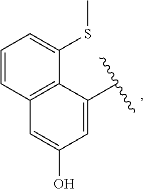 [SMILES: *c1cc(O)cc2cccc(SC)c12]

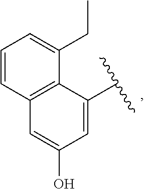 [SMILES: *c1cc(O)cc2cccc(CC)c12]

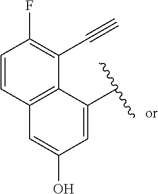 [SMILES: *c1cc(O)cc2ccc(F)c(C#C)c12]

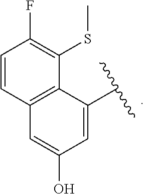 [SMILES: *c1cc(O)cc2ccc(F)c(SC)c12]

[27]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [26], wherein, R₅₁ is selected from hydrogen, deuterium, —F, —Cl, —Br, —C₁₋₃alkyl, haloC₁₋₃alkyl, haloC₁₋₃alkoxy, —CN, —NHC₁₋₃alkyl, —N(C₁₋₃alkyl)₂, —OC₁₋₃alkyl, —O-(3-6 membered cycloalkyl), —SC₁₋₃alkyl, —S(haloC₁₋₃alkyl) or 3-6 membered cycloalkyl; wherein, said —C₁₋₃alkyl or 3-6 membered cycloalkyl is optionally substituted with 1, 2 or 3 substituents selected from halogen, —C₁₋₃alkyl, haloC₁₋₃alkyl, haloC₁₋₃alkoxy, —CN, —NH₂, —NH(C₁₋₃alkyl), —N(C₁₋₃alkyl)₂, —OH, —OC₁₋₃alkyl, —SH, —SC₁₋₃alkyl or —S(haloC₁₋₃alkyl).

[28]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [27], wherein, R₅₁ is selected from hydrogen, deuterium, —Cl, —CN, —CH₃, —CHF₂, —CH₂F, —CF₃, —OH, —CH₂OH, —CH₂CH₃, —OCH₃, —OCH₂CH₃, —OCH(CH₃)₂, —SCH₃, —NHCH₃, —N(CH₃)₂, —OCF₃, —CN, —CH₂CN, —COOH, —CONH₂, —S(═O)CH₃, —S(═O)₂CH₃, —COOCH₃,

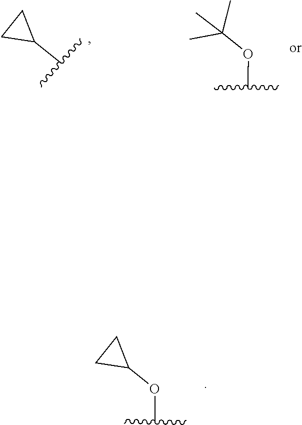

[29]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [28], wherein, R₅₁ is selected from hydrogen.

[30]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [29], wherein, R₅₂ is selected from halogen.

[31]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [30], wherein, R₅₂ is selected from —F.

[32]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [31], wherein, the prodrug comprises a prodrug moiety attached to R₄ and the prodrug moiety is capable of being converted to —OH.

[33]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [32], wherein, the prodrug is selected from any one of the following formulas:

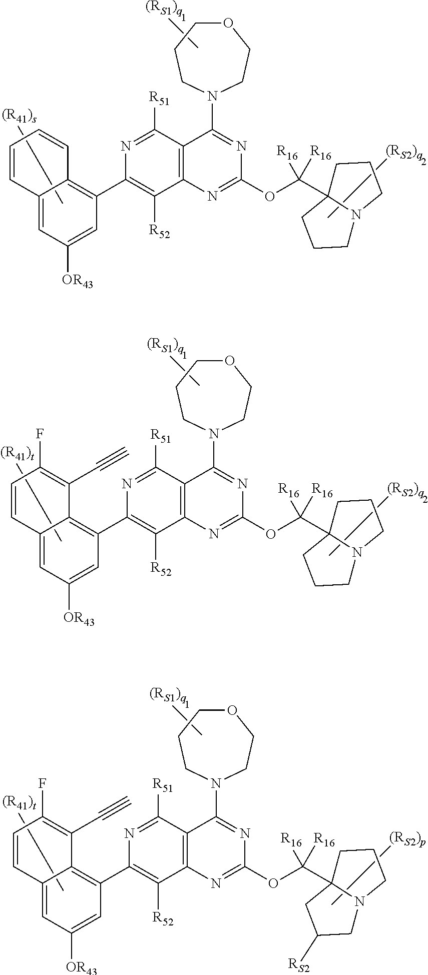

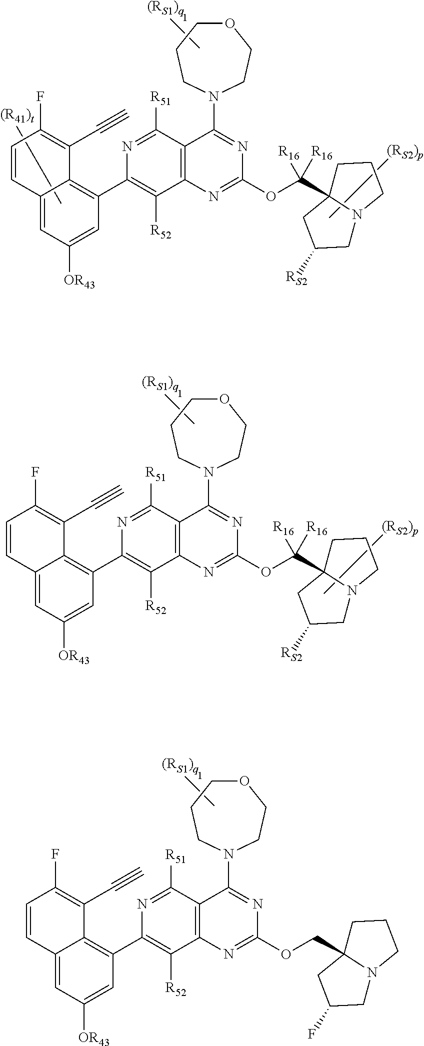

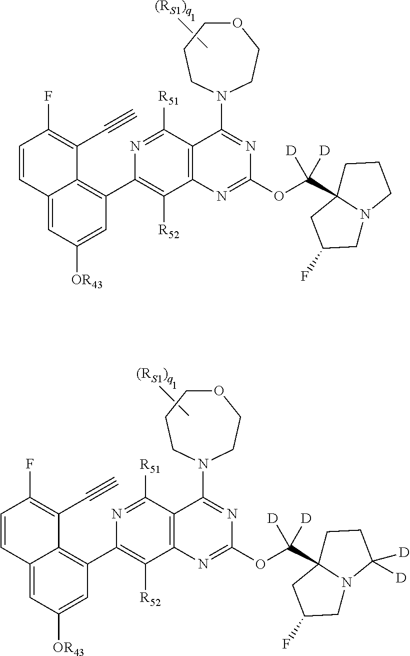

R₄₃ at each occurrence is independently selected from

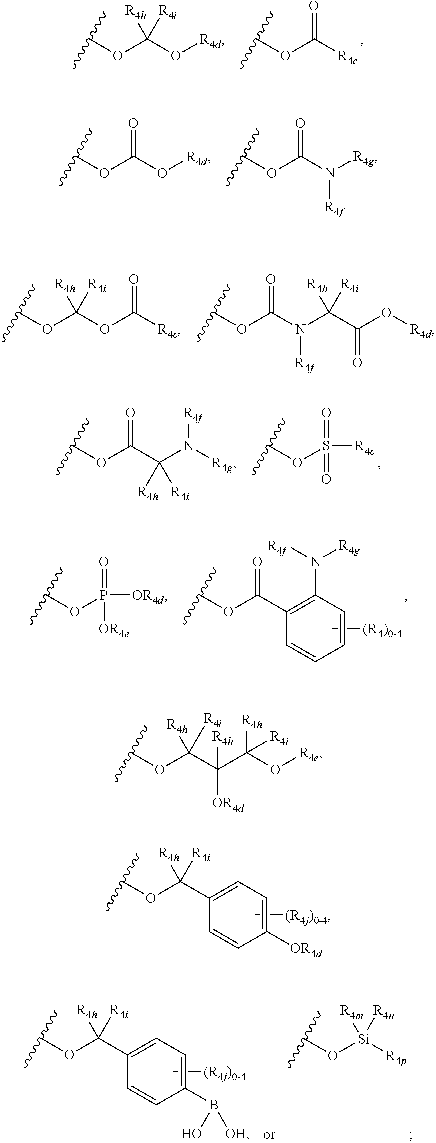

R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄₁;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄₁;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄₁;
Optionally, R_{4f} and R₄ₕ together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄₁;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂ or —OC₁₋₆alkyl;
Each of (heterocyclyl and heteroaryl) at each occurrence is independently contain 1, 2, 3 or 4 heteroatoms selected from N, O, S, S(═O) or S(═O)₂.

[34]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [33], —OR₄₃ is selected from any moiety in the Table 11:

TABLE 11

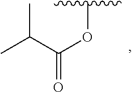 [SMILES: *OC(=O)C(C)C]

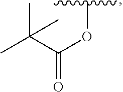

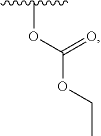 [SMILES: *OC(=O)OCC]

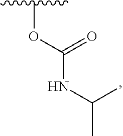

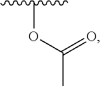 [SMILES: *OC(C)=O]

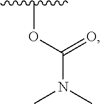 [SMILES: *OC(=O)N(C)C]

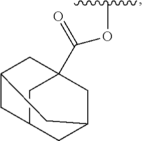

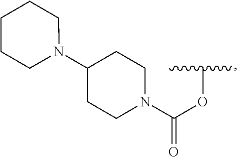

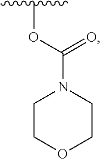 [SMILES: *OC(=O)N1CCOCC1]

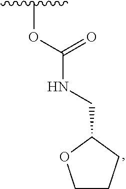 [SMILES: *OC(=O)NC[C@@H]1CCCO1]

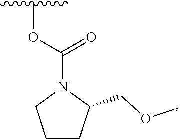

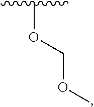

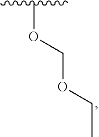 [SMILES: *OCOCC]

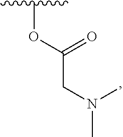

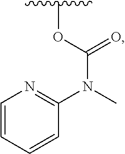 [SMILES: *OC(=O)N(C)c1ccccn1]

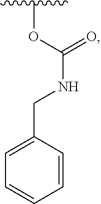 [SMILES: *OC(=O)NCc1ccccc1]

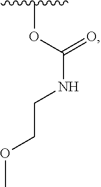 [SMILES: *OC(=O)NCCOC]

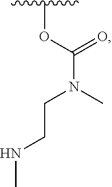 [SMILES: *OC(=O)N(C)CCNC]

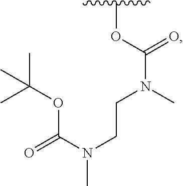 [SMILES: *OC(=O)N(C)CCN(C)C(=O)OC(C)(C)C]

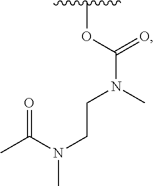 [SMILES: *OC(=O)N(C)CCN(C)C(C)=O]

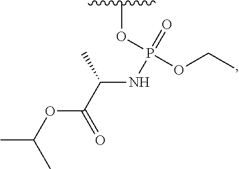

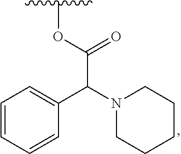 [SMILES: *OC(=O)C(c1ccccc1)N1CCCCC1]

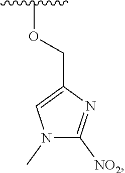 [SMILES: *OCc1cn(C)c([N+](=O)[O-])n1]

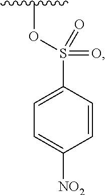 [SMILES: *OS(=O)(=O)c1ccc([N+](=O)[O-])cc1]

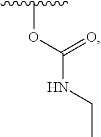 [SMILES: *OC(=O)NCC]

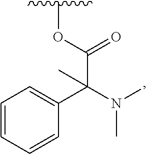

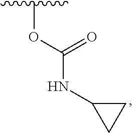 [SMILES: *OC(=O)NC1CC1]

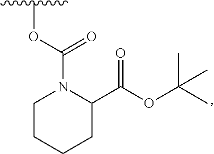 [SMILES: *OC(=O)N1CCCCC1C(=O)OC(C)(C)C]

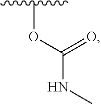 [SMILES: *OC(=O)NC]

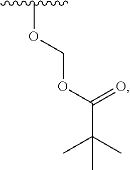 [SMILES: *OCOC(=O)C(C)(C)C]

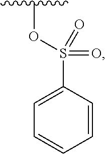 [SMILES: *OS(=O)(=O)c1ccccc1]

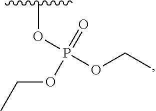

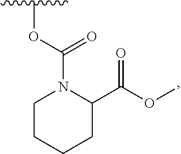

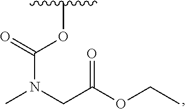 [SMILES: *OC(=O)N(C)CC(=O)OCC]

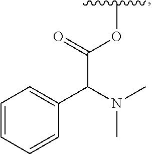

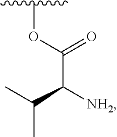 [SMILES: *OC(=O)[C@@H](N)C(C)C]

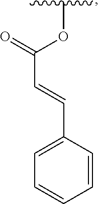

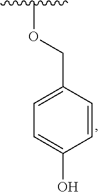 [SMILES: *OCc1ccc(O)cc1]

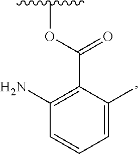

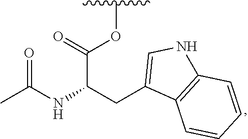 [SMILES: *OC(=O)[C@H](Cc1c[nH]c2ccccc12)NC(C)=O]

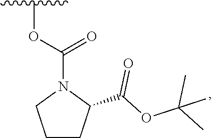

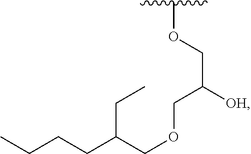 [SMILES: *OCC(O)COCC(CC)CCCC]

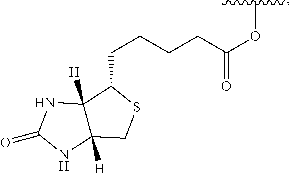

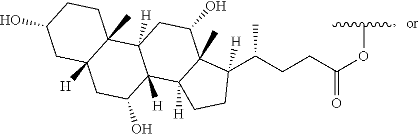

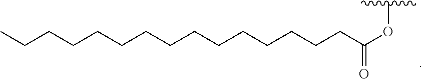 [SMILES: *OC(=O)CCCCCCCCCCCCCCC]

[35]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a drug thereof, a deuterated molecule thereof or a conjugated form thereof of [34] wherein, the moiety of

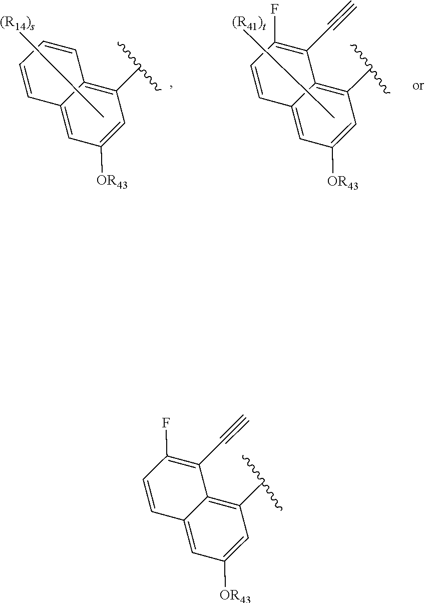

is selected from any moiety in the Table 12:

TABLE 12

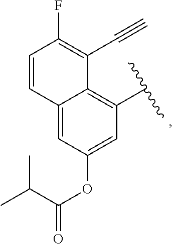 [SMILES: *c1cc(OC(=O)C(C)C)cc2ccc(F)c(C#C)c12]

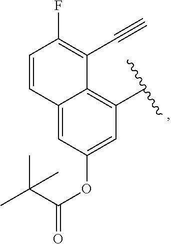 [SMILES: *c1cc(OC(=O)C(C)(C)C)cc2ccc(F)c(C#C)c12]

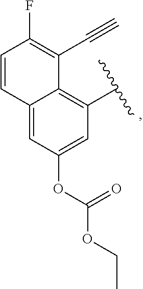 [SMILES: *c1cc(OC(=O)OCC)cc2ccc(F)c(C#C)c12]

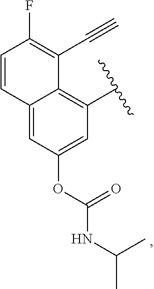 [SMILES: *c1cc(OC(=O)NC(C)C)cc2ccc(F)c(C#C)c12]

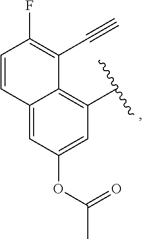 [SMILES: *c1cc(OC(C)=O)cc2ccc(F)c(C#C)c12]

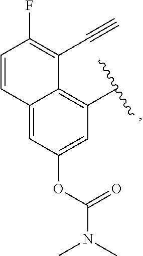 [SMILES: *c1cc(OC(=O)N(C)C)cc2ccc(F)c(C#C)c12]

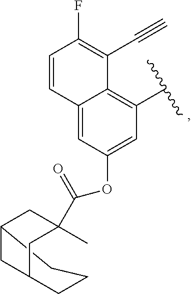 [SMILES: *c1cc(OC(=O)C2(C)CC3CCCC(C3)C2)cc2ccc(F)c(C#C)c12]

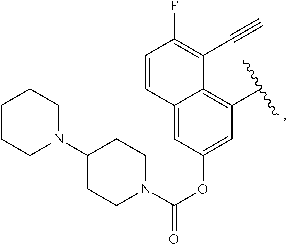 [SMILES: *c1cc(OC(=O)N2CCC(N3CCCCC3)CC2)cc2ccc(F)c(C#C)c12]

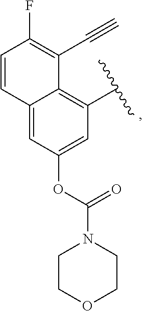 [SMILES: *c1cc(OC(=O)N2CCOCC2)cc2ccc(F)c(C#C)c12]

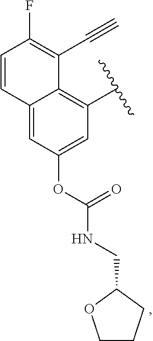 [SMILES: *c1cc(OC(=O)NC[C@@H]2CCCO2)cc2ccc(F)c(C#C)c12]

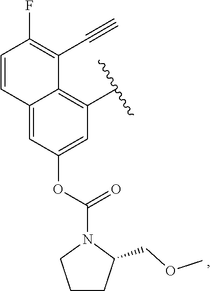 [SMILES: *c1cc(OC(=O)N2CCC[C@H]2COC)cc2ccc(F)c(C#C)c12]

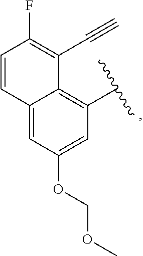 [SMILES: *c1cc(OCOC)cc2ccc(F)c(C#C)c12]

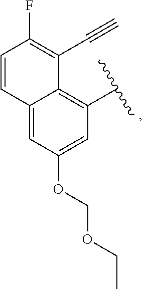 [SMILES: *c1cc(OCOCC)cc2ccc(F)c(C#C)c12]

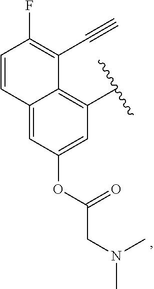 [SMILES: *c1cc(OC(=O)CN(C)C)cc2ccc(F)c(C#C)c12]

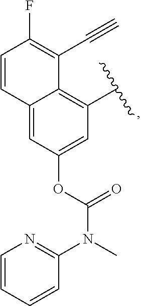 [SMILES: *c1cc(OC(=O)N(C)c2ccccn2)cc2ccc(F)c(C#C)c12]

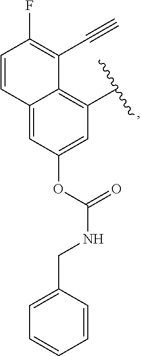 [SMILES: *c1cc(OC(=O)NCc2ccccc2)cc2ccc(F)c(C#C)c12]

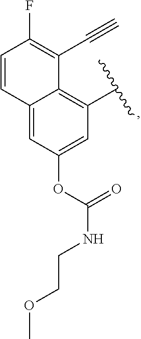 [SMILES: *c1cc(OC(=O)NCCOC)cc2ccc(F)c(C#C)c12]

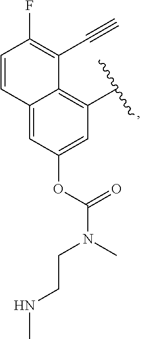 [SMILES: *c1cc(OC(=O)N(C)CCNC)cc2ccc(F)c(C#C)c12]

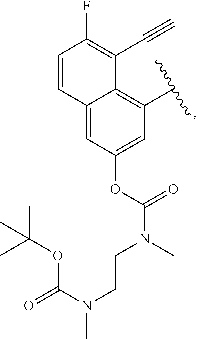 [SMILES: *c1cc(OC(=O)N(C)CCN(C)C(=O)OC(C)(C)C)cc2ccc(F)c(C#C)c12]

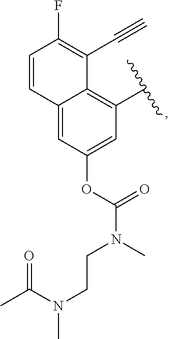 [SMILES: *c1cc(OC(=O)N(C)CCN(C)C(C)=O)cc2ccc(F)c(C#C)c12]

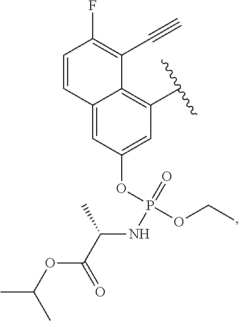

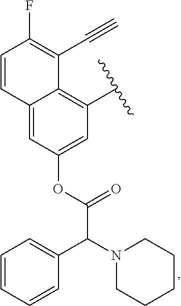 [SMILES: *c1cc(OC(=O)C(c2ccccc2)N2CCCCC2)cc2ccc(F)c(C#C)c12]

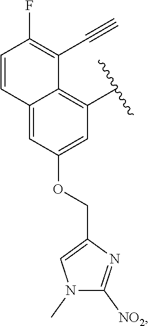 [SMILES: *c1cc(OCc2cn(C)c([N+](=O)[O-])n2)cc2ccc(F)c(C#C)c12]

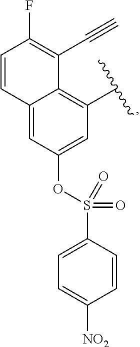 [SMILES: *c1cc(OS(=O)(=O)c2ccc([N+](=O)[O-])cc2)cc2ccc(F)c(C#C)c12]

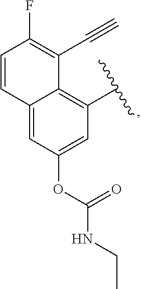 [SMILES: *c1cc(OC(=O)NCC)cc2ccc(F)c(C#C)c12]

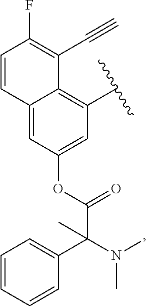

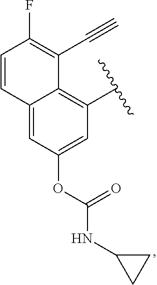 [SMILES: *c1cc(OC(=O)NC2CC2)cc2ccc(F)c(C#C)c12]

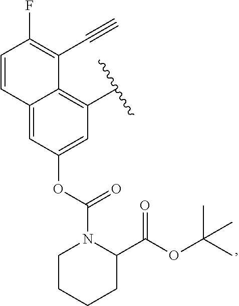 [SMILES: *c1cc(OC(=O)N2CCCCC2C(=O)OC(C)(C)C)cc2ccc(F)c(C#C)c12]

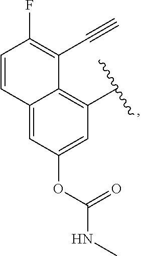 [SMILES: *c1cc(OC(=O)NC)cc2ccc(F)c(C#C)c12]

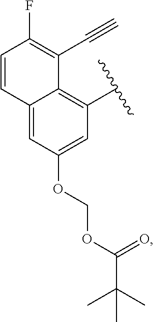 [SMILES: *c1cc(OCOC(=O)C(C)(C)C)cc2ccc(F)c(C#C)c12]

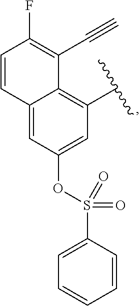 [SMILES: *c1cc(OS(=O)(=O)c2ccccc2)cc2ccc(F)c(C#C)c12]

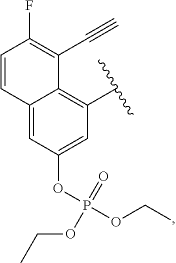

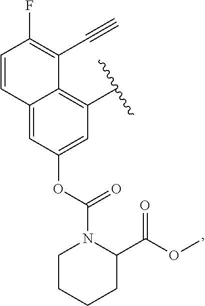

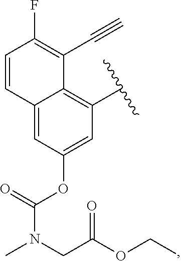

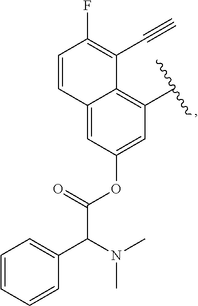 [SMILES: *c1cc(OC(=O)C(c2ccccc2)N(C)C)cc2ccc(F)c(C#C)c12]

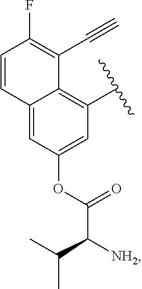 [SMILES: *c1cc(OC(=O)[C@@H](N)C(C)C)cc2ccc(F)c(C#C)c12]

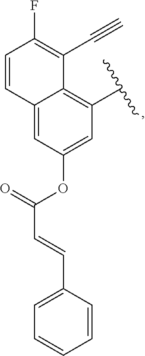 [SMILES: *c1cc(OC(=O)/C=C/c2ccccc2)cc2ccc(F)c(C#C)c12]

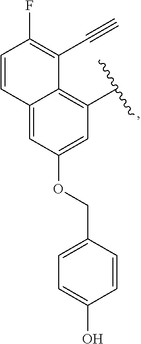 [SMILES: *c1cc(OCc2ccc(O)cc2)cc2ccc(F)c(C#C)c12]

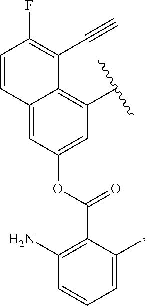

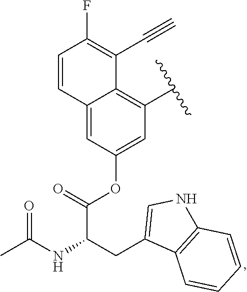 [SMILES: *c1cc(OC(=O)[C@H](Cc2c[nH]c3ccccc23)NC(C)=O)cc2ccc(F)c(C#C)c12]

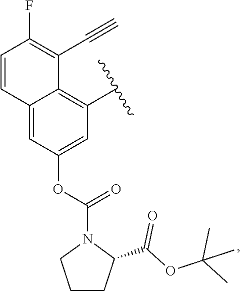 [SMILES: *c1cc(OC(=O)N2CCC[C@H]2C(=O)OC(C)(C)C)cc2ccc(F)c(C#C)c12]

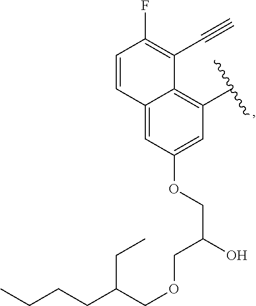 [SMILES: *c1cc(OCC(O)COCC(CC)CCCC)cc2ccc(F)c(C#C)c12]

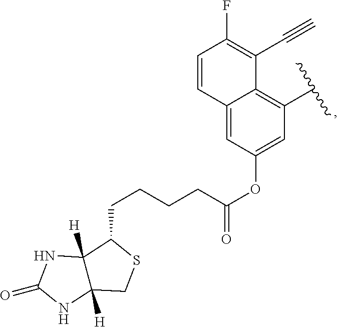 [SMILES: *c1cc(OC(=O)CCCC[C@@H]2SC[C@@H]3NC(=O)N[C@@H]32)cc2ccc(F)c(C#C)c12]

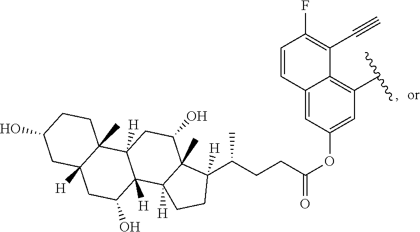 [SMILES: *c1cc(OC(=O)CC[C@@H](C)[C@H]2CC[C@H]3[C@@H]4[C@H](O)C[C@@H]5C[C@H](O)CC[C@]5(C)[C@H]4C[C@H](O)[C@]23C)cc2ccc(F)c(C#C)c12]

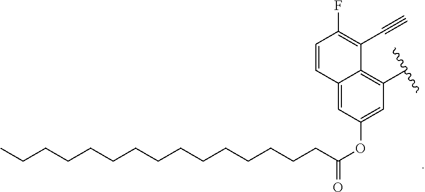 [SMILES: *c1cc(OC(=O)CCCCCCCCCCCCCCC)cc2ccc(F)c(C#C)c12]

[36]. A compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof:

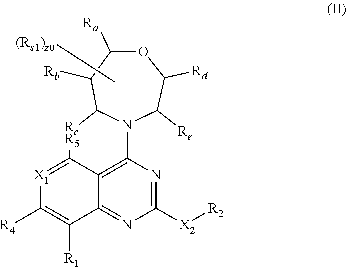

Wherein,
Rₐ and R_{b} together with the carbon atoms to which they are respectively attached form ring D, wherein, ring D is independently optionally substituted with one or more R_{S1}; R_{c}, R_{d}, and Rₑ, are hydrogen; or
R_{b} and R_{c} together with the carbon atoms to which they are respectively attached form ring E, wherein, ring E is independently optionally substituted with one or more R_{S1}; Rₐ, R_{d}, and Rₑ, are hydrogen; or
R_{d} and Rₑ together with the carbon atoms to which they are respectively attached form ring J, wherein, ring J is independently optionally substituted with one or more R_{S1}; Rₐ, R_{b}, and R_{c}, are hydrogen;
ring D, ring E, or ring J is a 3-10 membered carbocyclic ring, a 3-10 membered heterocyclic ring, a 6-10 membered aryl ring or a 5-10 membered heteroaryl ring;
R₁ is selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
X₁ is selected from CR₃ or N;
R₃ is selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
X₂ is selected from CR₂₁R₂₂, NR₂₃, O, S, SO or SO₂;
R₂₁ and R₂₂ are each independently selected from hydrogen, halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
R₂₃ is selected from hydrogen or —C₁₋₆alkyl;
R₂ is selected from

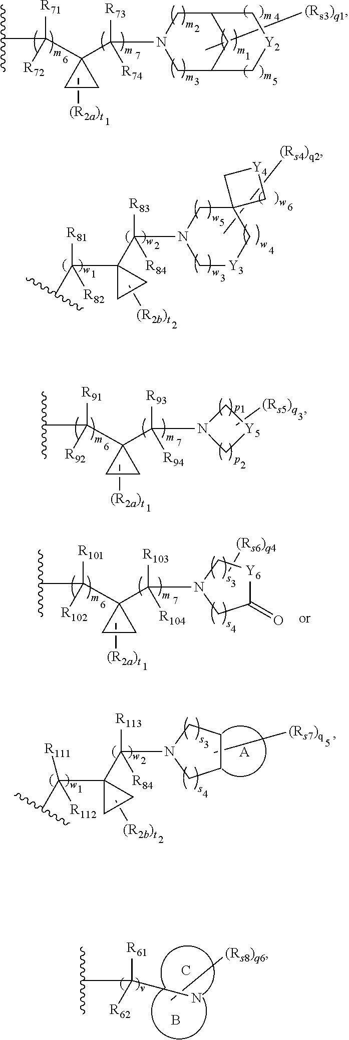

-L-(3-12 membered heterocyclyl), -L-(3-12 membered cycloalkyl), -L-(6-12 member aryl), -L-(5-12 membered heteroaryl) or -L-NR₂₄R₂₅;
Each L is independently selected from a bond or C₁₋₁₀ alkylene optionally substituted with one or more R_{S9};
R₂₄ and R₂₅ are each independently selected from hydrogen or —C₁₋₁₀alkyl optionally substituted with one or more R_{S10};
Said 3-12 membered heterocyclyl in -L-(3-12 membered heterocyclyl) is optionally substituted with one or more R_{S11};
Said 3-12 membered cycloalkyl in -L-(3-12 membered cycloalkyl) is optionally substituted with one or more R_{S12};
Said 6-12 member aryl in -L-(6-12 member aryl) is optionally substituted with one or more R_{S13};
Said 5-12 membered heteroaryl in -L-(5-12 membered heteroaryl) is optionally substituted with one or more R_{S14};
Y₂ is selected from O, S, SO, SO₂, C═O, NH or CH₂, when Y₂ is selected from NH or CH₂, the NH or CH₂ is optionally substituted with 1 or 2 R_{S3};
m₁, m₂, m₃, m₄ or m₅ is independently selected from 0, 1, 2, 3, 4, 5 or 6;
m₆ or m₇ is independently selected from 0, 1, 2, 3, 4, 5 or 6;
Y₃ and Y₄ are each independently selected from O, S, SO, SO₂, C═O, NH or CH₂, when Y₃ and Y₄ is selected from NH or CH₂, the NH or CH₂ is optionally substituted with 1 or 2 R_{S4};
w₁ and w₂ are each independently selected from 0, 1, 2, 3, 4, 5 or 6;
w₃, w₄, w₅, w₆ and w₇ are each independently selected from 0, 1, 2, 3, 4, 5 or 6; provided that w₆ and w₇ are not 0 at the same time;
Y₅ is selected from O, S, SO, SO₂, C═O, NH or CH₂, when Y₅ is selected from NH or CH₂, the NH or CH₂ is optionally substituted with 1 or 2 Rₛ₅;
p₁ and p₂ are each independently selected from 0, 1, 2, 3, 4, 5, or 6, provided that p₁ and p₂ are not 0 at the same time;
p₃ and p₄ are each independently selected from 0, 1, 2, 3, 4, 5 or 6;
Y₆ is selected from O, S, NH or CH₂, when Y₆ is selected from NH or CH₂, the NH or CH₂ is optionally substituted with 1 or 2 R_{S6};
s₁ and s₂ are each independently selected from 0, 1, 2, 3, 4, 5 or 6;
s₃ and s₄ are each independently selected from 0, 1, 2, 3, 4, 5, or 6, provided that s₃ and s₄ are not 0 at the same time;
r₁ and r₂ are each independently selected from 0, 1, 2, 3, 4, 5 or 6;
r₃ and r₄ are each independently selected from 0, 1, 2, 3, 4, 5, or 6;
v is selected from 0, 1, 2, 3, 4, 5 or 6;
ring A is selected from a 3-10 membered carbocyclic ring, a 3-10 membered heterocyclic ring, a 6-10 membered aryl ring or a 5-10 membered heteroaryl ring, said heterocyclic or heteroaryl ring at each occurrence is independently contains 1, 2, 3 or 4 heteroatoms selected from N, O or S;
Ring B and ring C are each independently selected from a 3-10 membered heterocyclic ring which is optionally further contains 1, 2, or 3 heteroatoms selected from N, O or S except the fused N atom;
R_{S1}, R_{S3}, R_{S4}, R_{S5}, R_{S6}, R_{S7}, R_{S8}, R_{S9}, R_{S10}, R_{S11}, R_{S12}, R_{S13} and R_{S14} are each independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more R_{Sa};
Each of R_{Sa} is independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, phenyl or 5-6 membered heteroaryl; wherein said 3-6 membered cycloalkyl, 3-6 membered heterocyclyl, phenyl or 5-6 membered heteroaryl is independently optionally substituted with 1, 2, or 3 R_{Sb};
Each of R_{Sb} is independently selected from halogen; —C₁₋₆alkyl; haloC₁₋₆alkyl; —CN; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂ or —OC₁₋₆alkyl;
q₁, q₂, q₃, q₄, q₅ and q₆ are each independently selected from 0, 1, 2, 3, 4, 5 or 6;
R₂ₐ, R_{2b}, R_{2c}, R_{2d} and R₂ₑ are each independently selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
t₁, t₂, t₃, t₄ and t₅ are each independently selected from 0, 1, 2, 3, 4, 5 or 6;
R₆₁, R₆₂, R₇₁, R₇₂, R₇₃, R₇₄, R₈₁, R₈₂, R₈₃, R₈₄, R₉₁, R₉₂, R₉₃, R₉₄, R₁₀₁, R₁₀₂, R₁₀₃, R₁₀₄, R₁₁₁, R₁₁₂, R₁₁₃ and R₁₁₄ are each independently selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
R₄ is selected from 6-10 membered aryl, 5-10 membered heteroaryl,

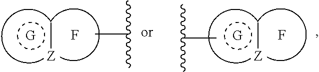

wherein said 6-10 membered aryl, 5-10 membered heteroaryl,

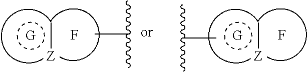

is optionally independently substituted with one or more R₄ₐ;
Z at each occurrence is independently selected from C or N;
Ring G at each occurrence is independently selected from a 6 membered aryl ring or a 5-6 membered heteroaryl ring and ring F at each occurrence is a 3-10 membered carbocyclic ring or a 3-10 membered heterocyclic ring when Z is selected from C;
Ring G at each occurrence is selected from a 5-6 membered heteroaryl ring and ring F at each occurrence is a 3-10 membered heterocyclic ring when Z is selected from N;
Each of R₄ₐ is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)H, —C(═O)(C₁₋₆alkyl), —C(═O)OH, —C(═O)(OC₁₋₆alkyl), —OC(═O)(C₁₋₆alkyl), —C(═O)NH₂, —NO₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, 5-10 membered heteroaryl or R₄₁, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 R_{4b};
Each of R_{1b} is independently selected from halogen; —C₁₋₆alkyl; haloC₁₋₆alkyl; —CN; oxo; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂, —OC₁₋₆alkyl or cyclopropyl;
R₄₁ is selected from

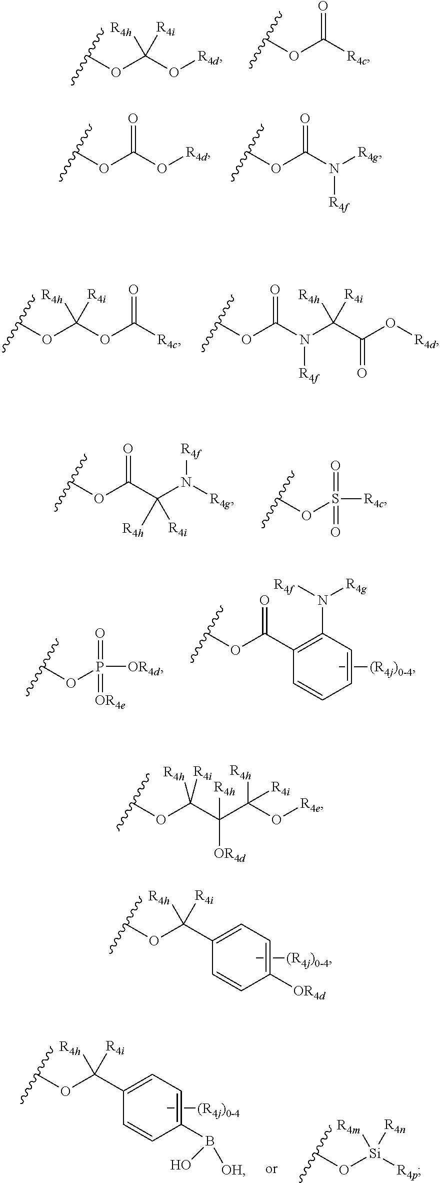

R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄ⱼ;
Optionally, R_{4f} and R₄ₕ together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄ⱼ;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂ or —OC₁₋₆alkyl;
R₅ is selected from hydrogen, halogen, —C₁₋₆alkyl, haloC₁₋₆alkyl, haloC₁₋₆alkoxy, —C₂₋₆alkenyl, —C₂₋₆alkynyl, —CN, —NR₅₁R₅₂, —OR₅₃, —SR₅₄, —S(haloC₁₋₆alkyl), —S(═O)R₅₅, —S(═O)₂R₅₅, —C(═O)R₅₅, —C(═O)(OR₅₃), —OC(═O)(R₅₅), —C(═O)NR₅₁R₅₂, —NR₅₁C(═O)(R₅₅), —S(═O)₂NR₅₁R₅₂, —NR₅₁S(═O)₂R₅₅, 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NR₅₁R₅₂, —OR₅₃, —SR₅₄, —S(haloC₁₋₆alkyl), —S(═O)R₅₅, —S(═O)₂R₅₅, —C(═O)R₅₅, —C(═O)(OR₅₃), —OC(═O)(R₅₅), —C(═O)NR₅₁R₅₂, —NR₅₁C(═O)(R₅₅), —S(═O)₂NR₅₁R₅₂, —NR₅₁S(═O)₂R₅₅, 3-10 membered cycloalkyl, 3-10 membered heterocyclyl, 6-10 membered aryl or 5-10 membered heteroaryl;
R₅₁, R₅₂, R₅₃ and R₅₄ are each independently selected from hydrogen, —C₁₋₆alkyl, haloC₁₋₆alkyl, —NH₂, —S(═O)(C₁₋₆alkyl), —S(═O)₂(C₁₋₆alkyl), —C(═O)(C₁₋₆alkyl), —C(═O)OH, —C(═O)(OC₁₋₆alkyl), —C(═O)NH₂, —C(═O)NH(C₁₋₆alkyl), —C(═O)N(C₁₋₆alkyl)₂, —S(═O)₂NH₂, —S(═O)₂NH(C₁₋₆alkyl), —S(═O)₂N(C₁₋₆alkyl)₂, 3-10 membered cycloalkyl, 3-10 membered heterocyclyl, 6-10 membered aryl, or 5-10 membered heteroaryl, wherein, said —C₁₋₆alkyl, 3-10 membered cycloalkyl, 3-10 membered heterocyclyl, 6-10 membered aryl, or 5-10 membered heteroaryl is independently optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl;
R₅₅ is selected from hydrogen, —C₁₋₆alkyl, haloC₁₋₆alkyl, haloC₁₋₆alkoxy, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —OC(═O)(C₁₋₆alkyl), —NHC(═O)(C₁₋₆alkyl), —N(C₁₋₆alkyl)C(═O)(C₁₋₆alkyl), —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein, said —C₁₋₆alkyl, 3-10 membered cycloalkyl, 3-10 membered heterocyclyl, 6-10 membered aryl, or 5-10 membered heteroaryl is independently optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl;
z₀ is independently selected from 0, 1, 2, 3, 4, 5 or 6;
Each of (heterocyclyl and heteroaryl) at each occurrence is independently contain 1, 2, 3 or 4 heteroatoms selected from N, O, S, S(═O) or S(═O)₂.

[37]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of [36], wherein, the compound is any one of the following formulas:

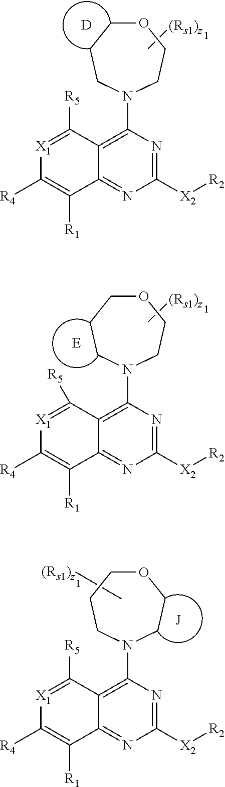

Wherein, ring D, ring E, or ring J is a 3-6 membered carbocyclic ring, a 3-6 membered heterocyclic ring containing 1, 2, or 3 ring members selected from —N—, —O—, or —S—, a phenyl or a 5-6 membered heteroaryl ring containing 1, 2, or 3 ring members selected from —N—, —O—, —S—;
z₁ at each occurrence is independently selected from 0, 1, 2, 3, 4, 5 or 6.

[38]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of [36] or [37], wherein the compound is any one of the following formulas in the Table 13:

TABLE 13

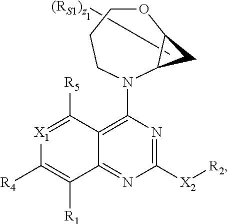

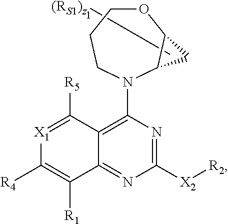

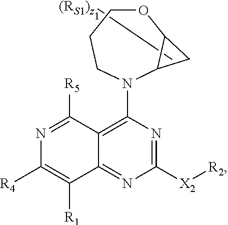

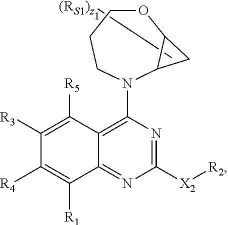

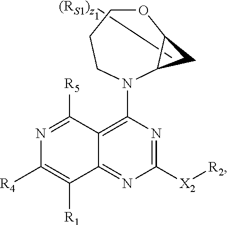

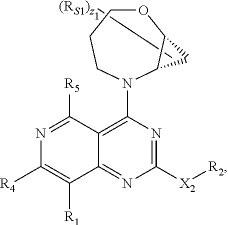

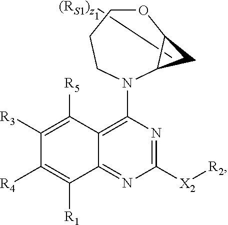

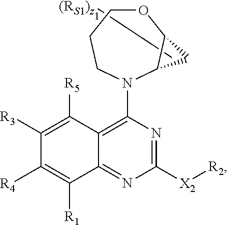

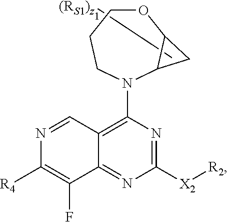

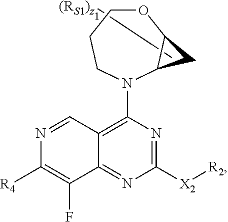

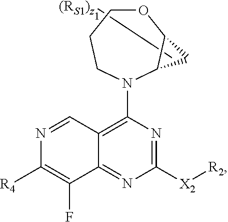

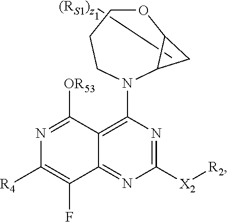

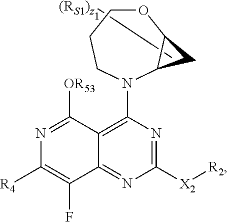

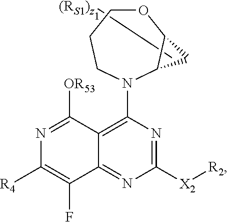

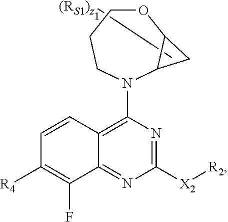

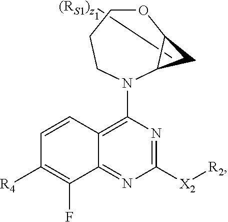

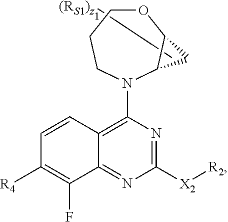

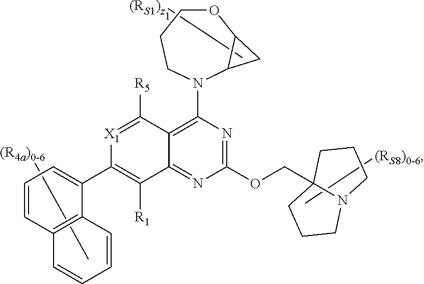

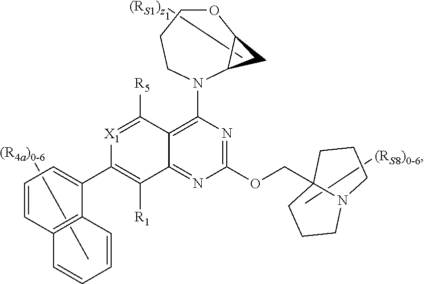

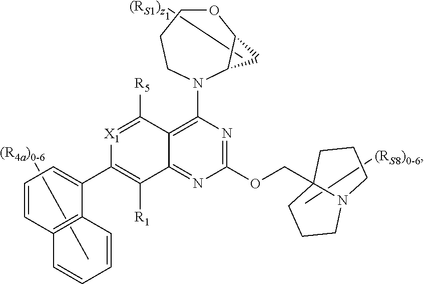

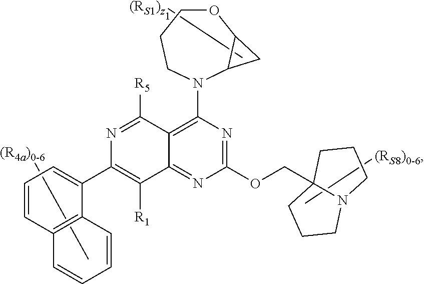

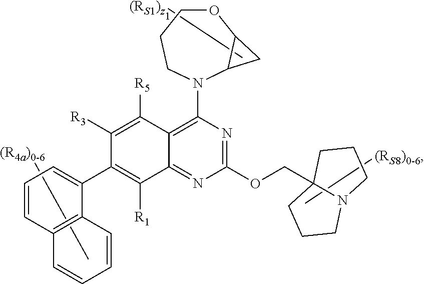

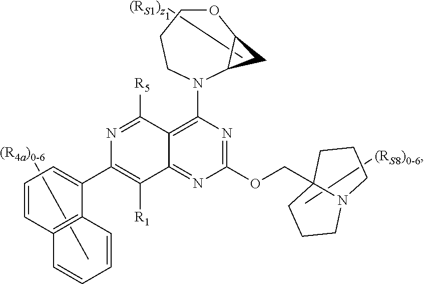

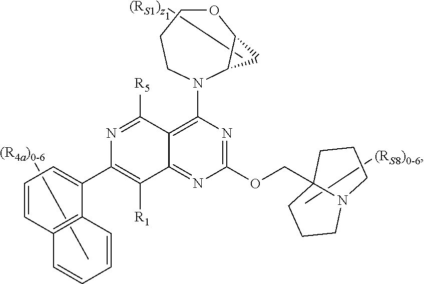

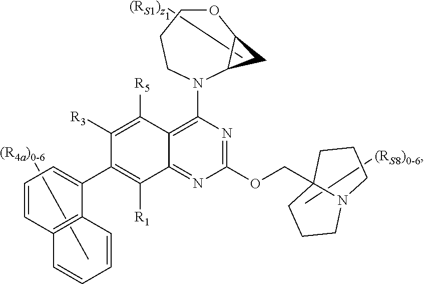

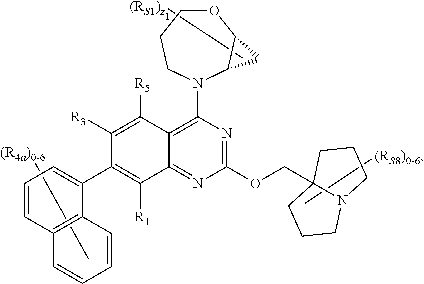

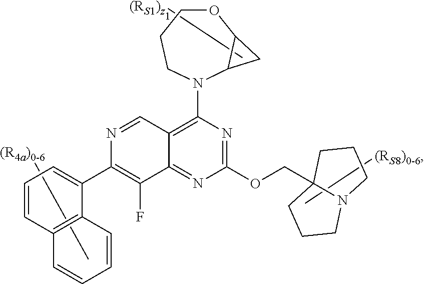

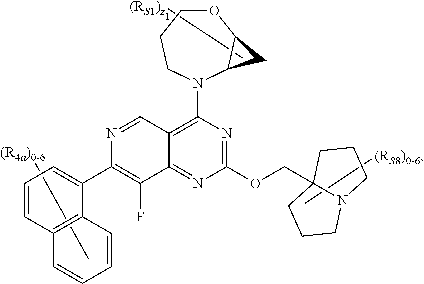

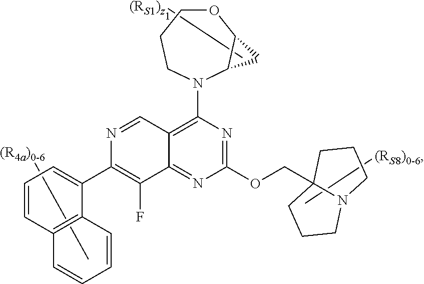

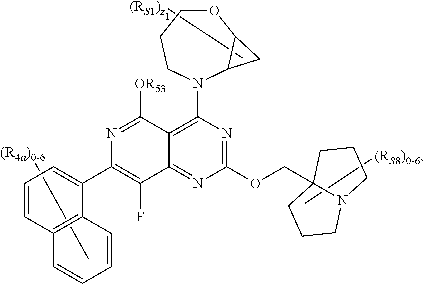

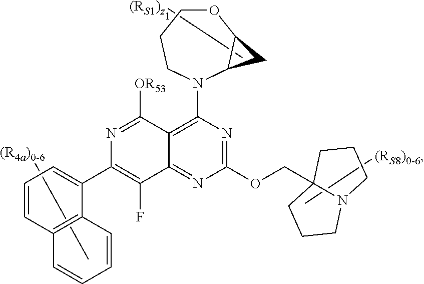

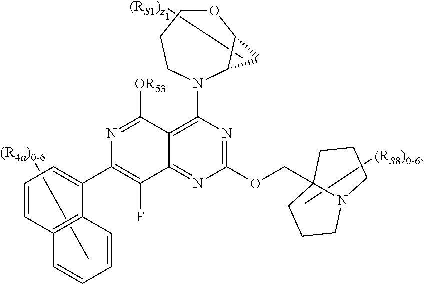

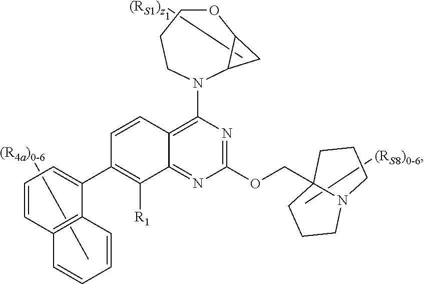

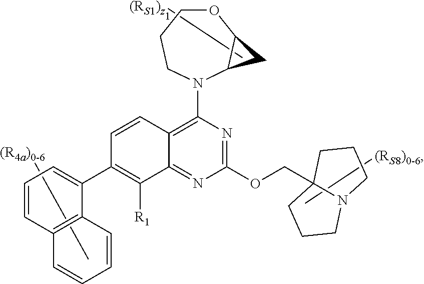

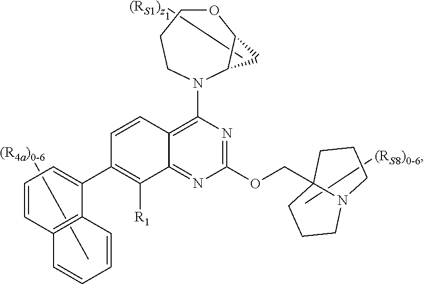

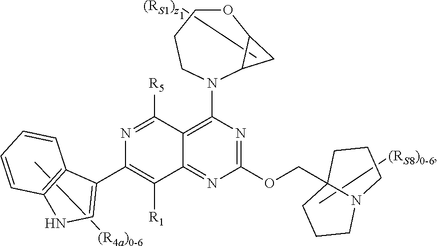

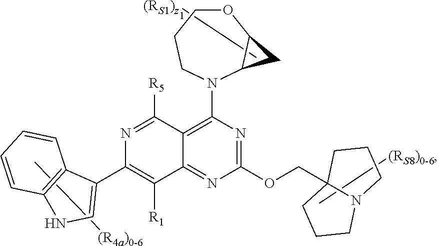

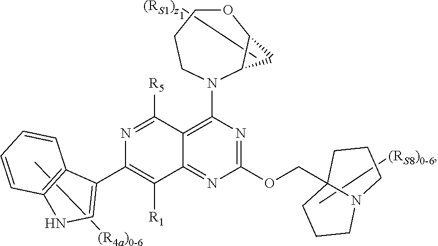

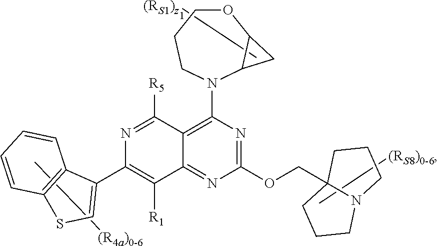

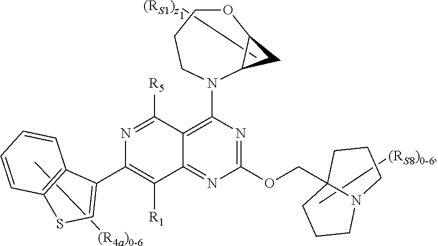

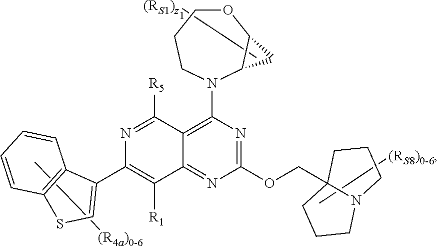

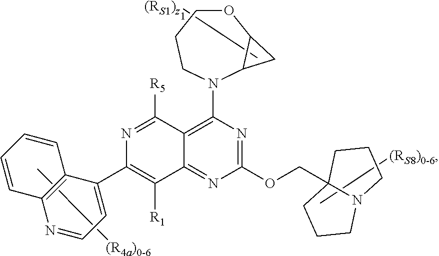

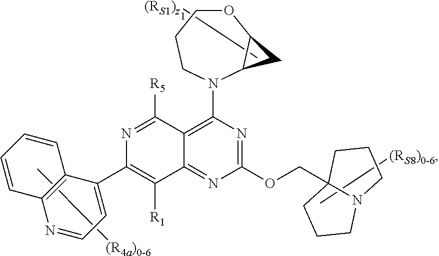

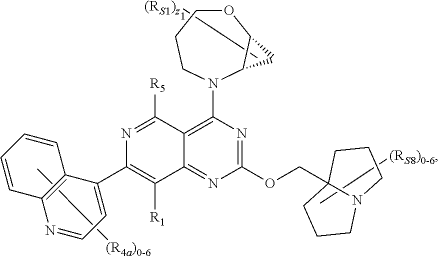

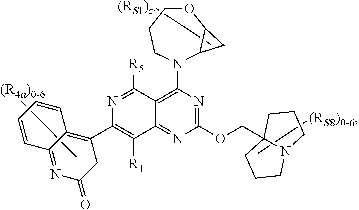

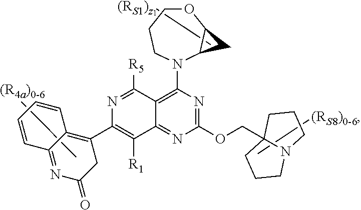

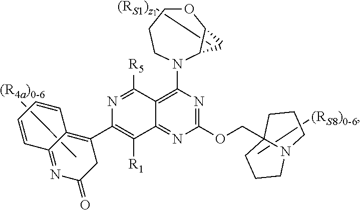

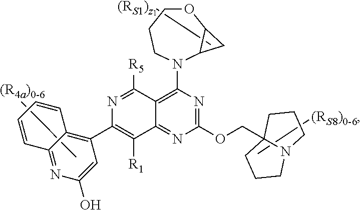

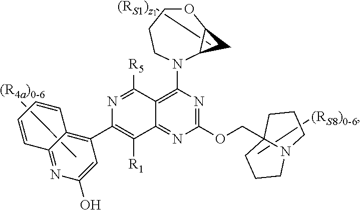

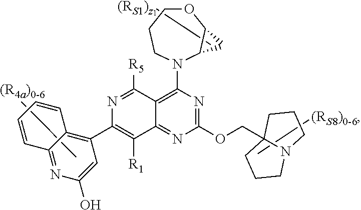

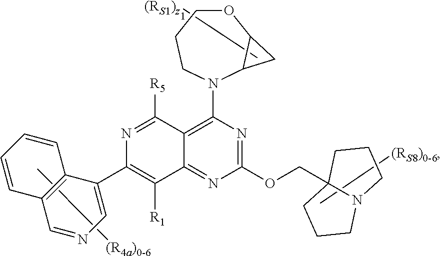

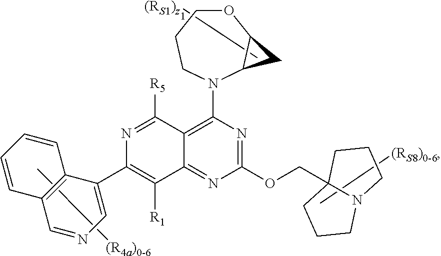

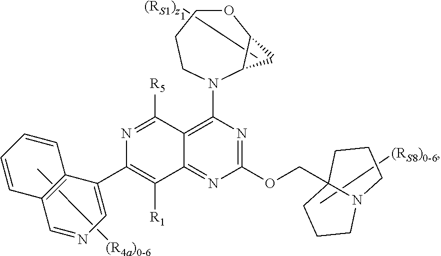

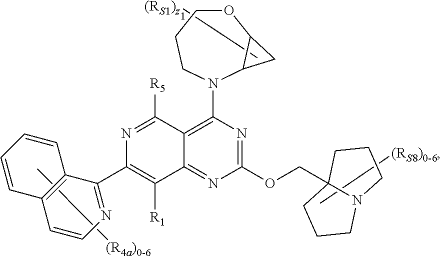

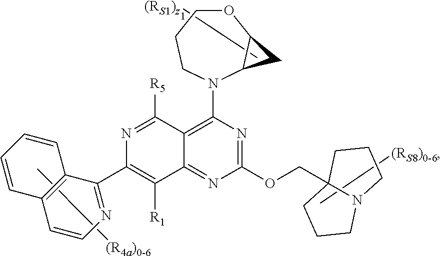

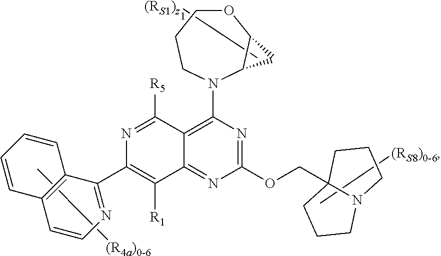

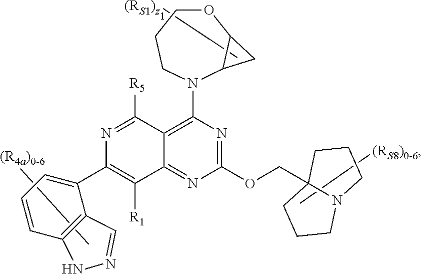

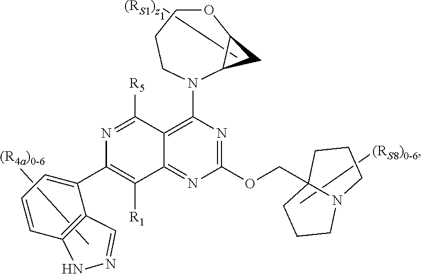

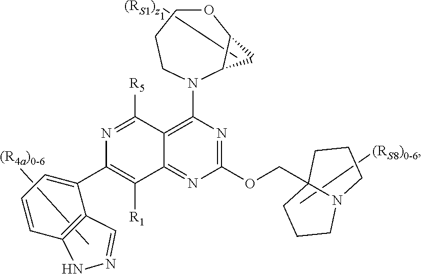

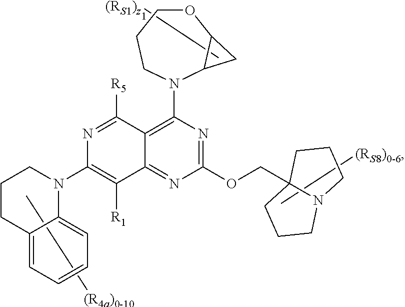

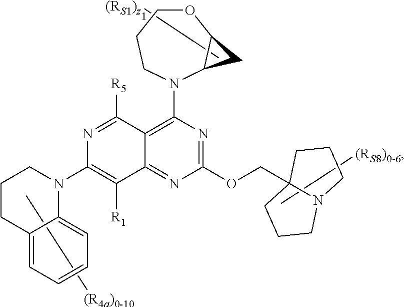

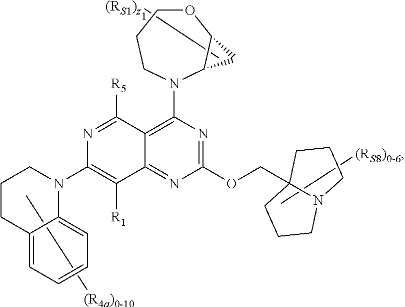

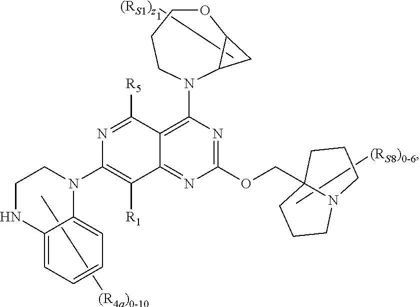

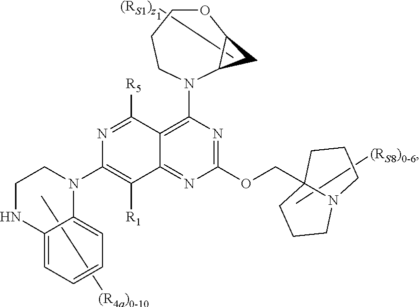

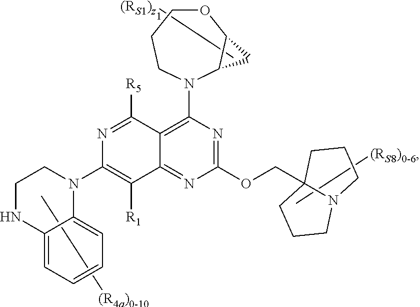

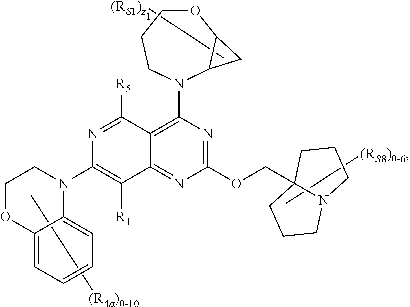

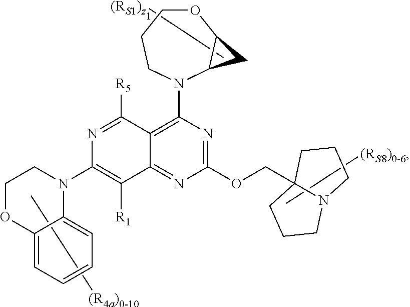

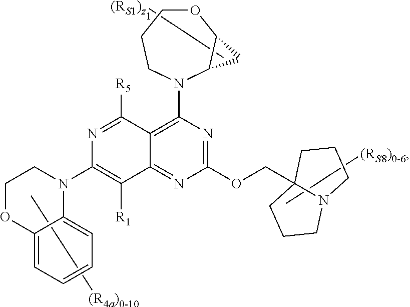

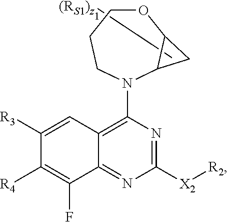

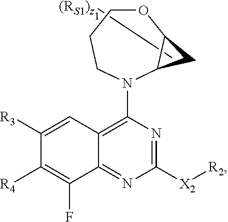

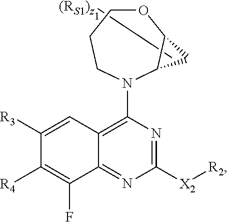

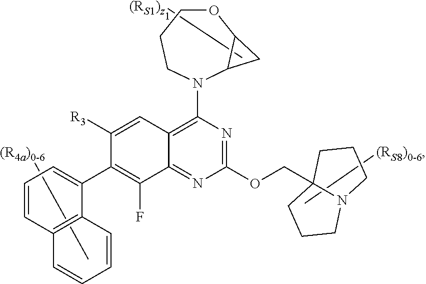

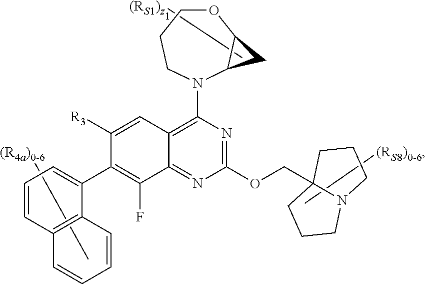

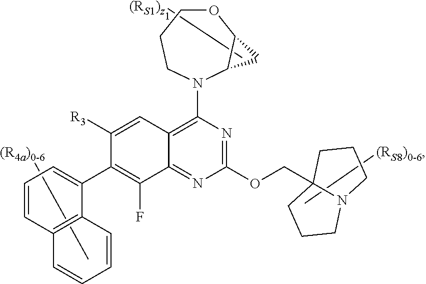

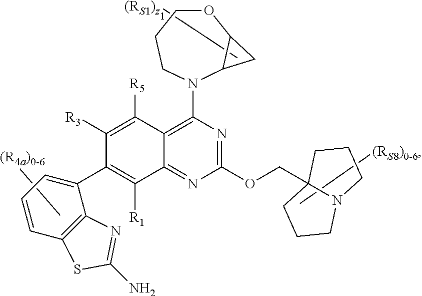

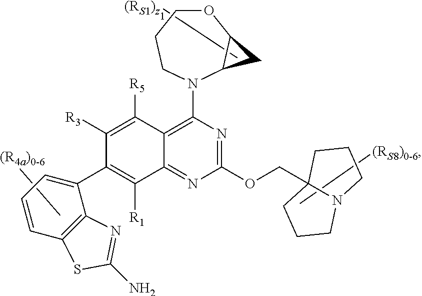

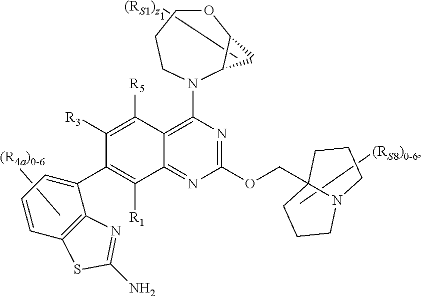

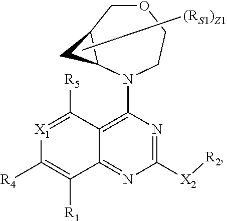

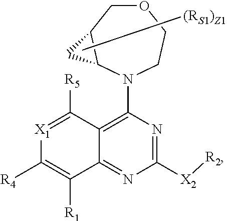

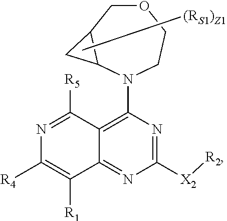

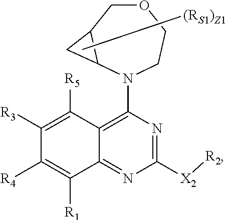

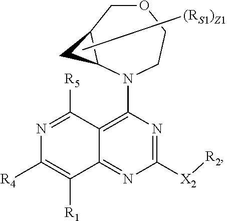

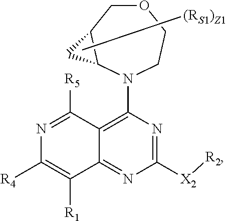

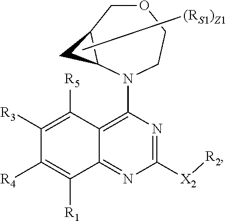

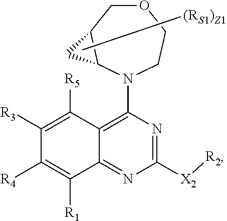

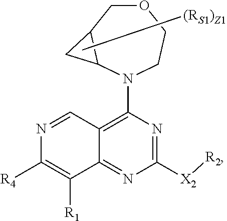

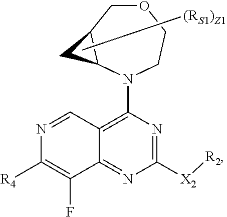

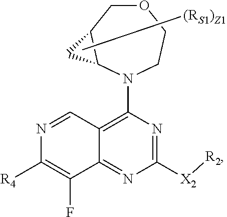

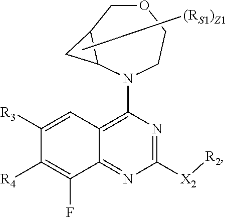

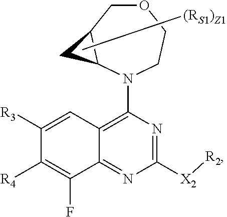

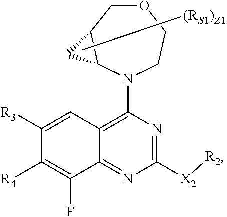

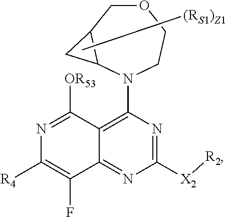

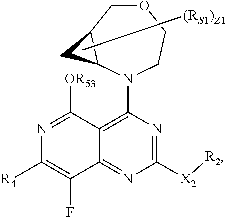

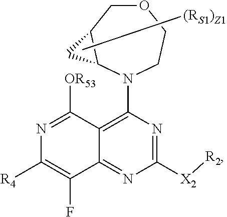

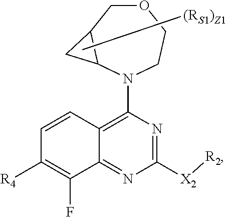

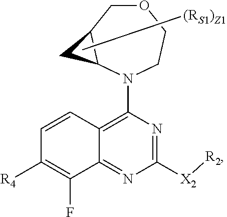

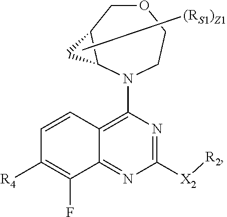

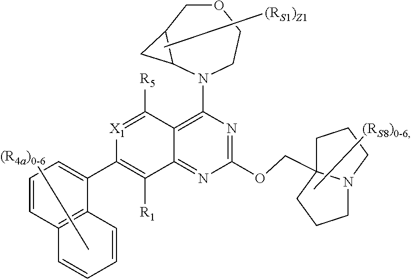

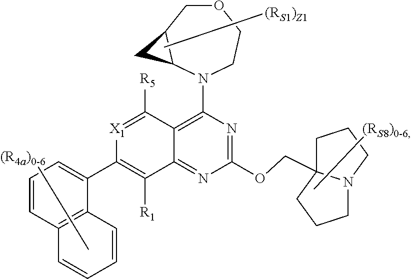

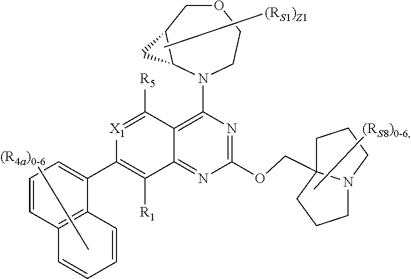

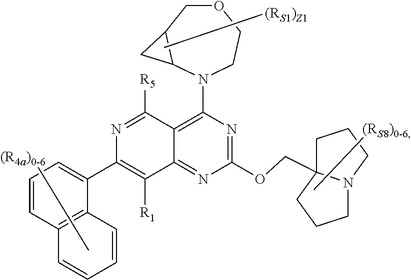

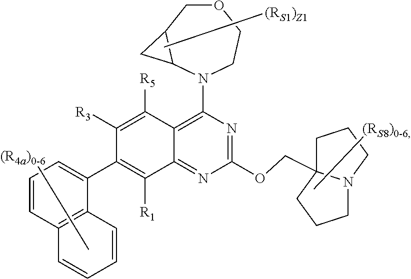

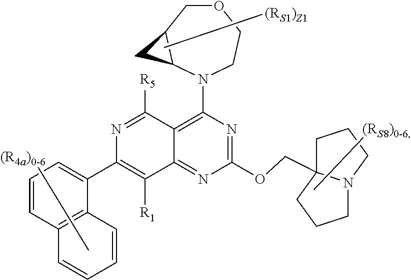

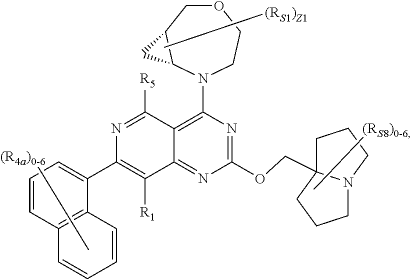

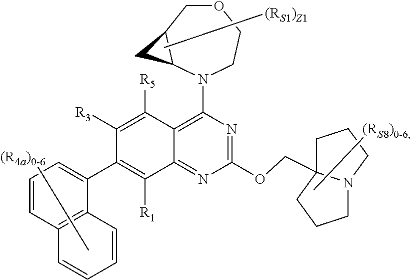

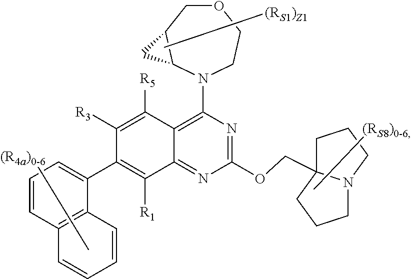

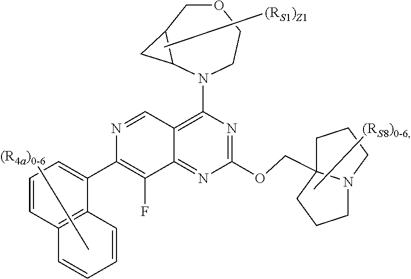

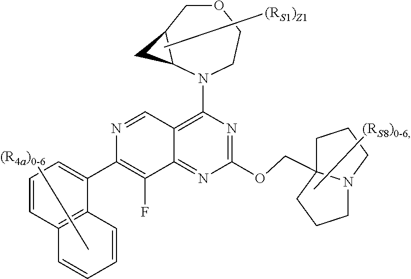

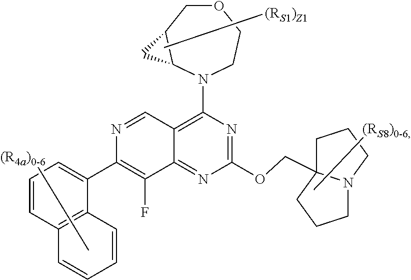

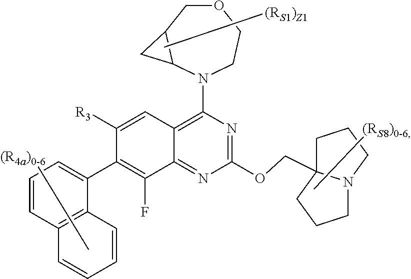

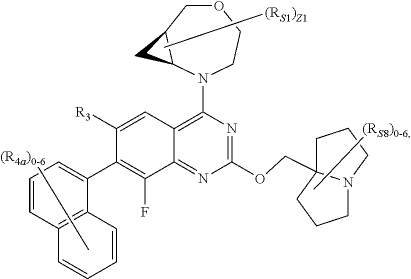

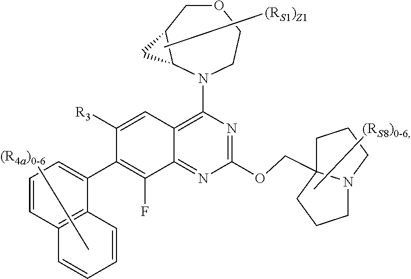

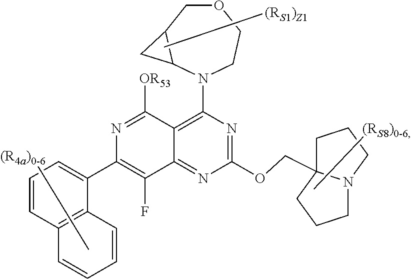

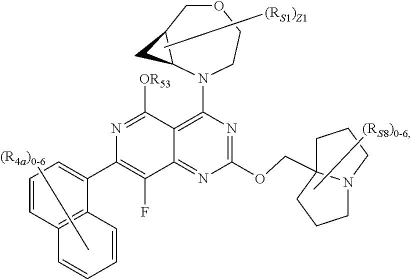

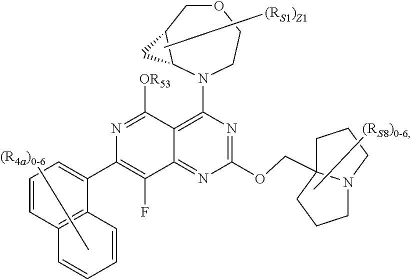

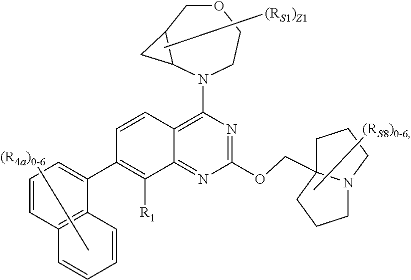

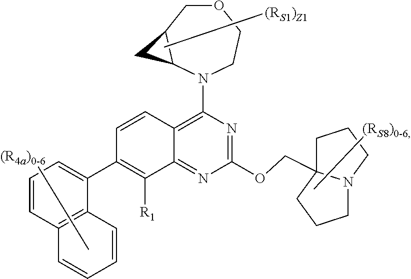

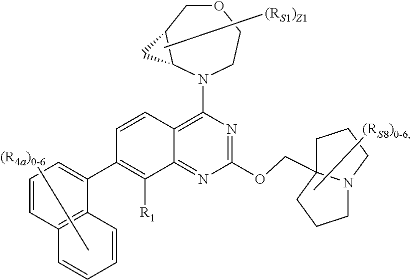

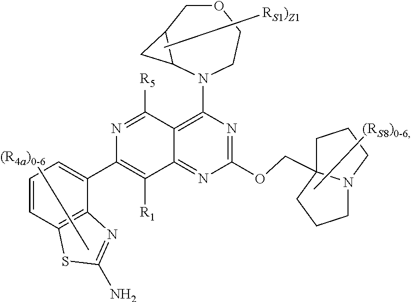

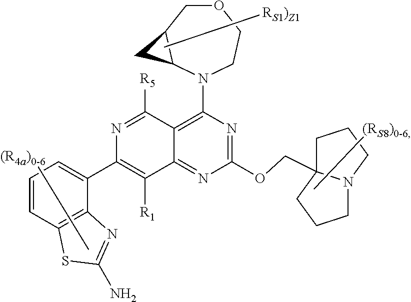

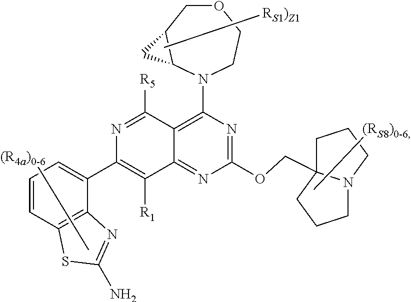

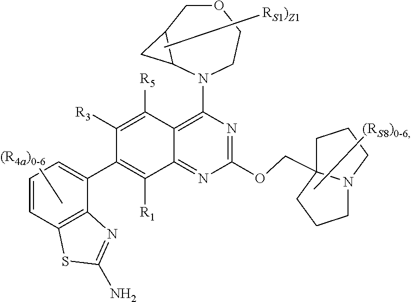

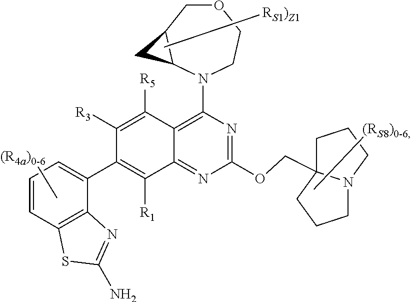

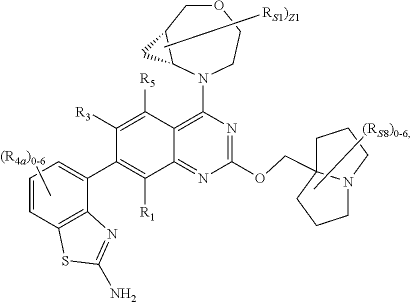

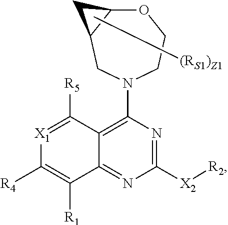

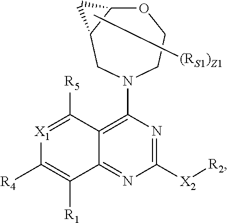

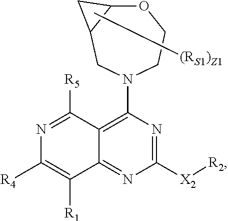

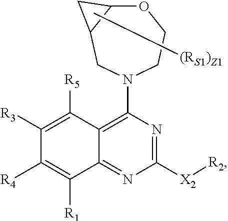

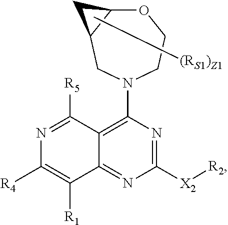

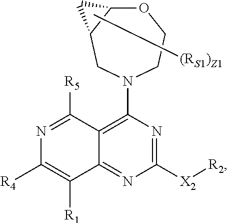

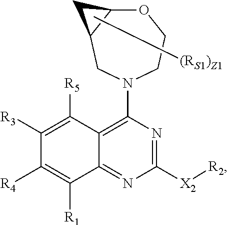

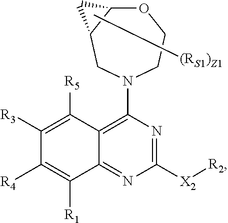

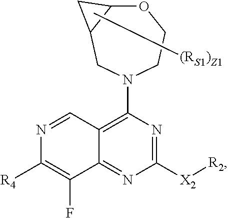

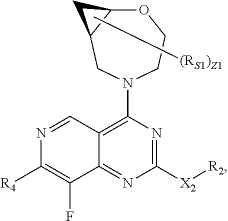

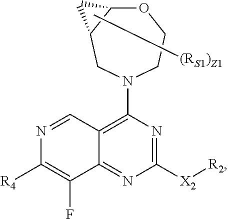

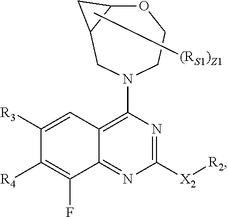

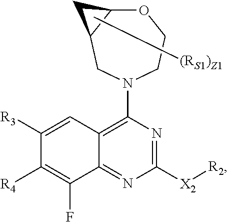

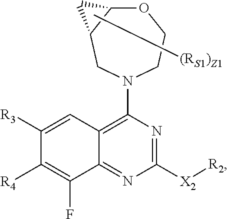

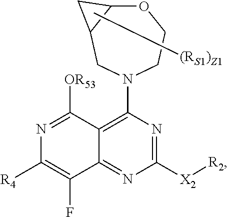

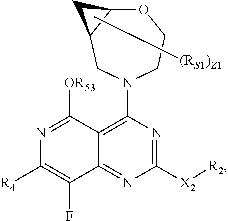

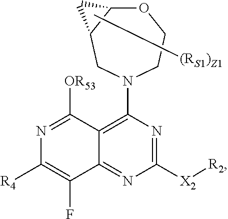

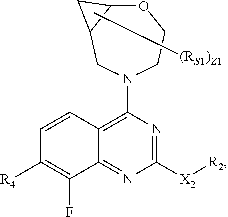

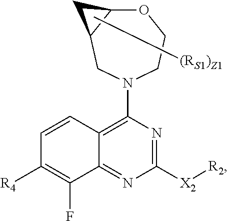

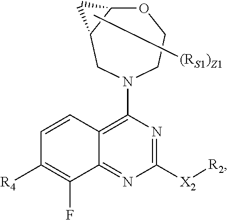

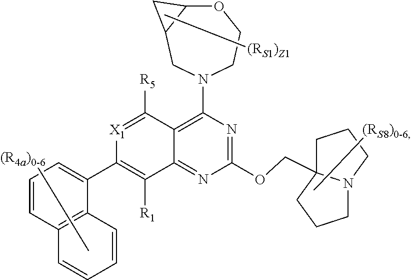

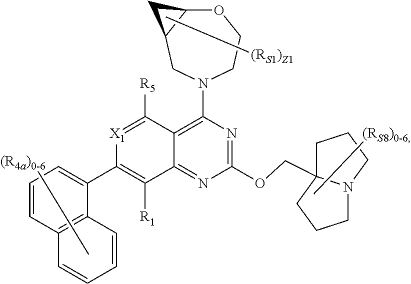

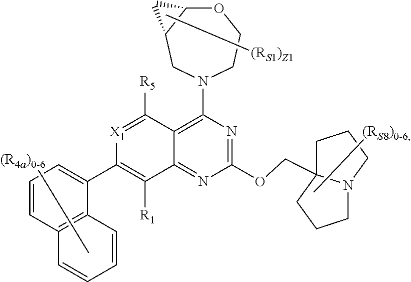

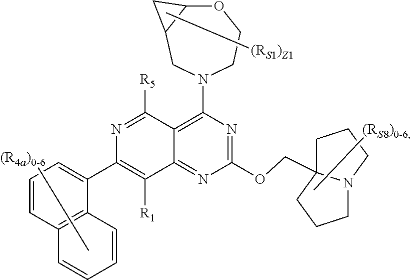

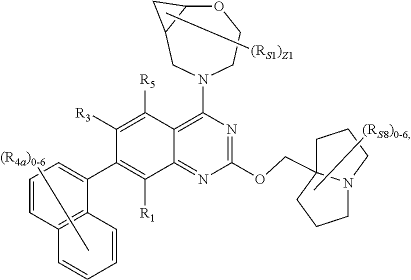

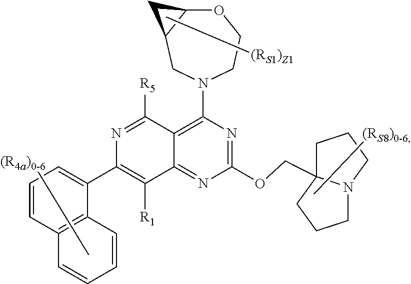

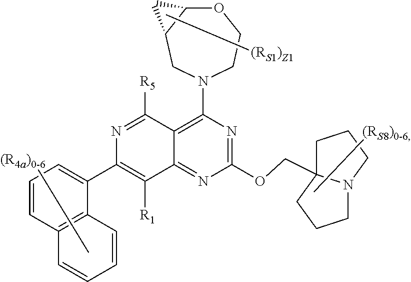

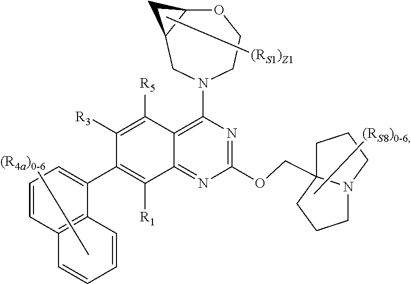

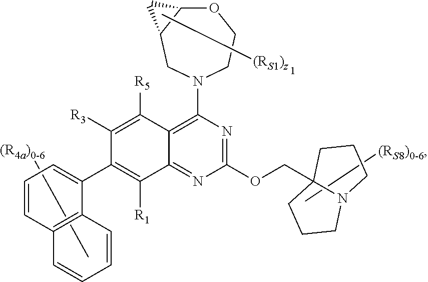

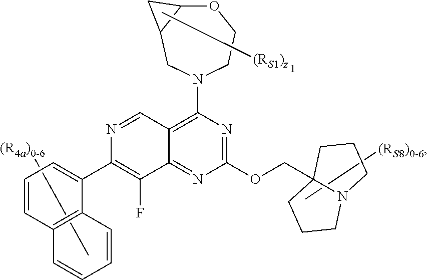

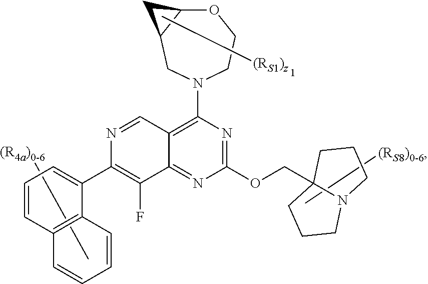

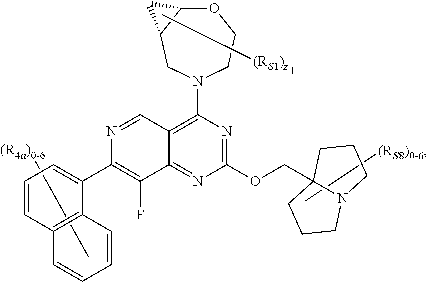

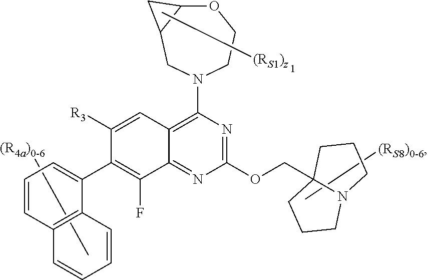

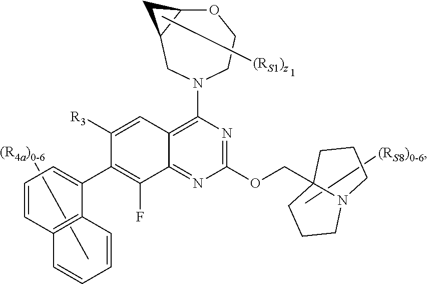

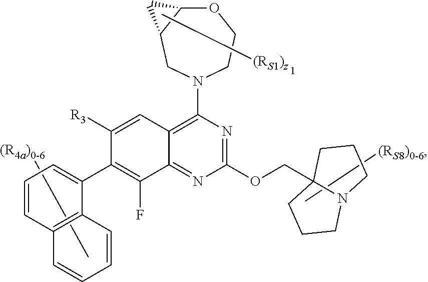

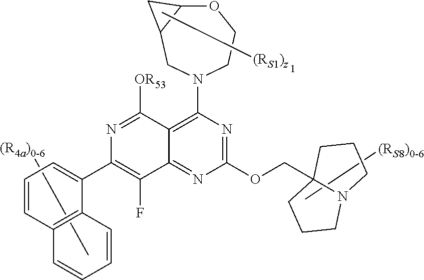

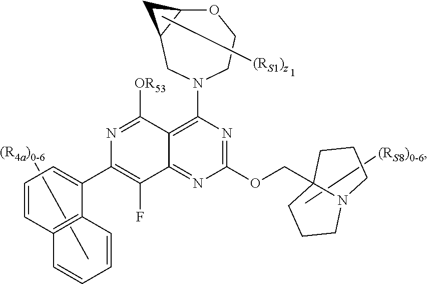

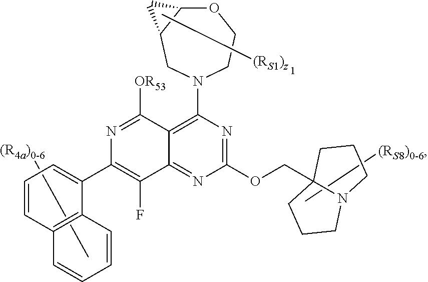

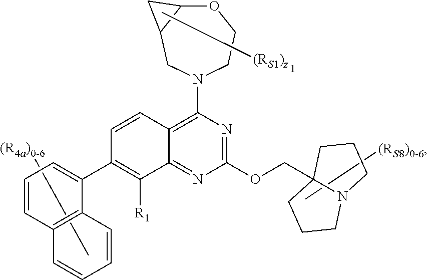

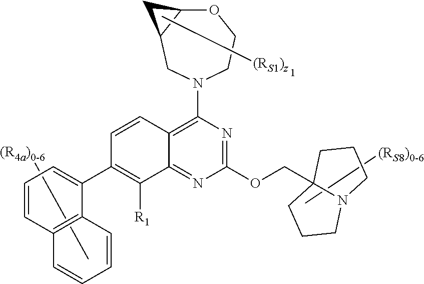

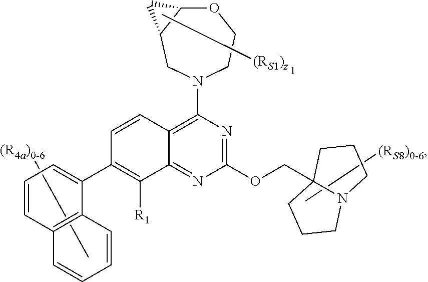

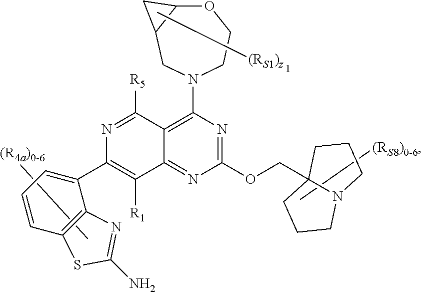

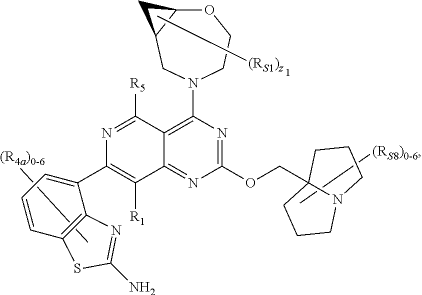

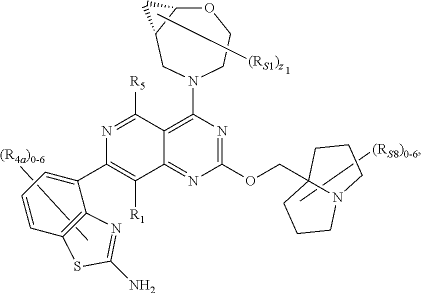

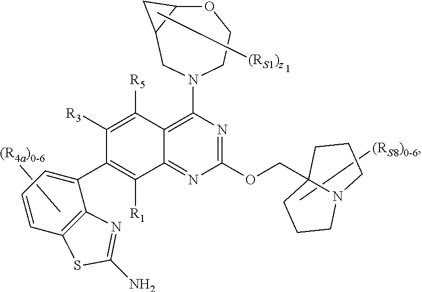

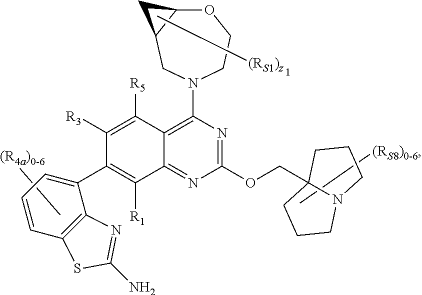

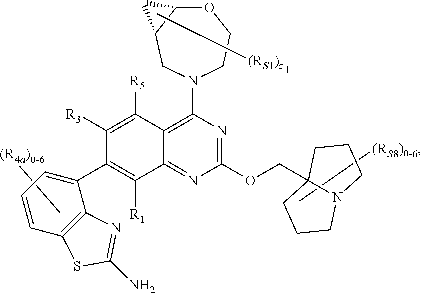

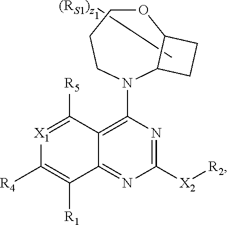

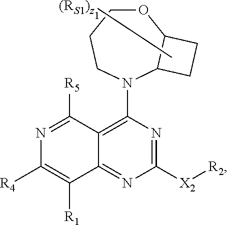

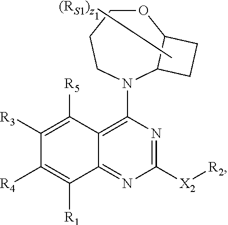

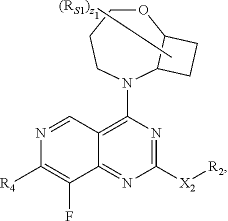

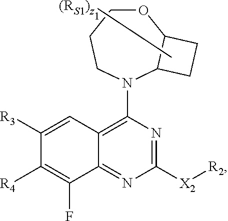

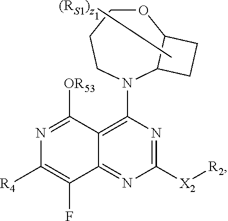

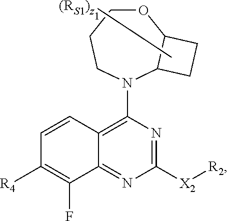

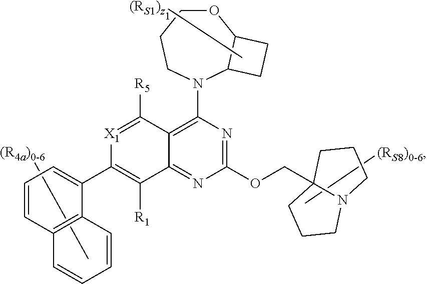

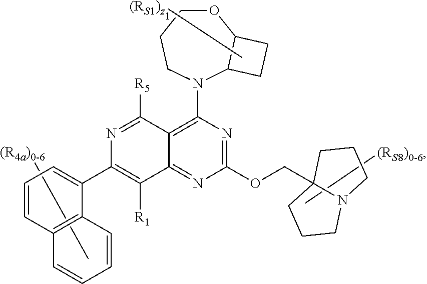

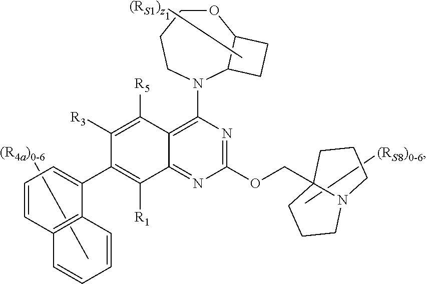

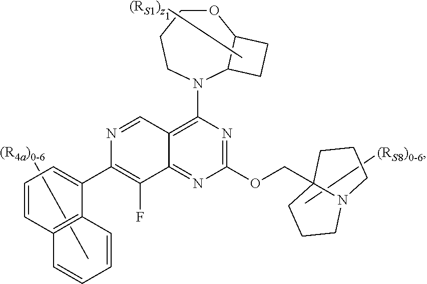

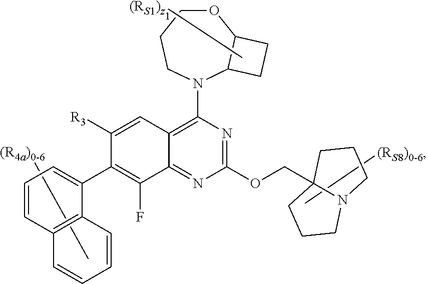

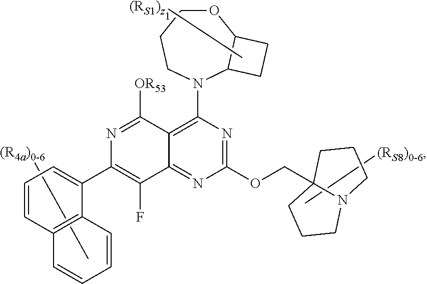

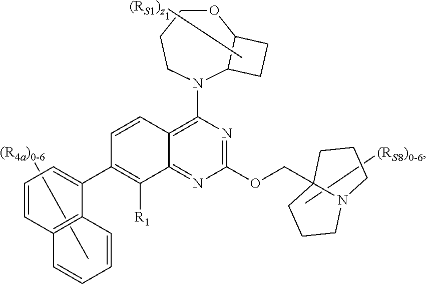

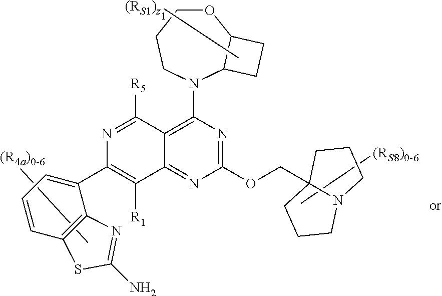

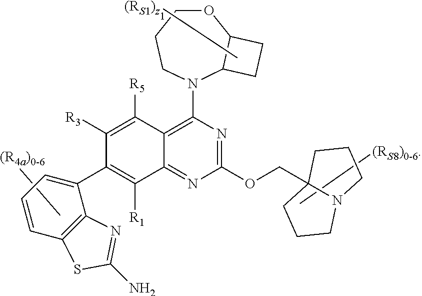

[39]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [38], wherein, R₁ is selected from hydrogen, —F, —Cl, —Br, —C₁₋₃alkyl, haloC₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 6-10 membered cycloalkyl, 6-10 membered heterocyclyl, 6-8 membered aryl or 5-8 membered heteroaryl.

[40]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [39], wherein, R₁ is selected from —H, —F, —Cl, —CH₃, —CH₂CH₃, —CN, —COOH, —CH₂OH, —OH, —OCH₃, —OCH₂CH₃, —CF₃, —CHF₂, —NH₂, —NHCH₃, —N(CH₃)₂, —CH₂NH₂, —CH₂CH₂NH₂, —CH₂OH, —CH₂CH₂OH, —SH, —S—CH₃, —CH₂SH, —CH₂CH₂SH, —CH═CH₂, —C═CH, —CHCH═CH₂, —OCF₃, —OCHF₂, —C(═O)NH₂, —C(═O)OCH₃,

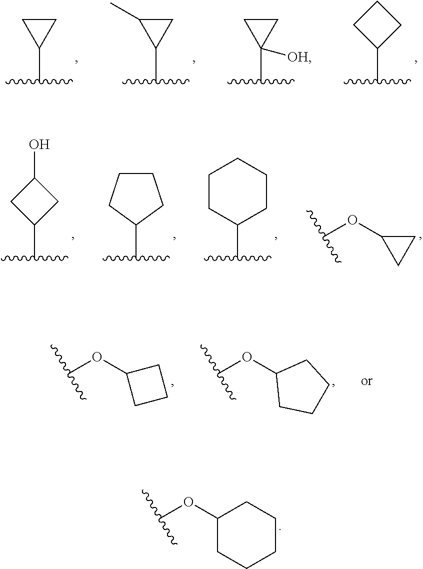

[41]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [40], wherein, R₁ is —H or —F.

[42]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [41], wherein, R₃ is selected from hydrogen, —F, —Cl, —Br, —C₁₋₃alkyl, haloC₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(haloC₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 3-10 membered cycloalkyl, 3-10 membered heterocyclyl, 6-10 membered aryl or 5-8 membered heteroaryl.

[43]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [42], wherein, R₃ is selected from —H, —F, —Cl, —CH₃, —CH₂CH₃, —CH(CH₃)₂, —CN, —COOH, —CH₂OH, —OH, —OCH₃, —OCH₂CH₃, —CF₃, —CHF₂, —NH₂, —NHCH₃, —N(CH₃)₂, —CH₂NH₂, —CH₂CH₂NH₂, —CH₂OH, —CH₂CH₂OH, —SH, —S—CH₃, —S—CF₃, —CH₂SH, —CH₂CH₂SH, —CH═CH₂, —C≡CH, —CHCH═CH₂, —OCF₃, —OCHF₂, —C(═O)NH₂, —C(═O)OCH₃,

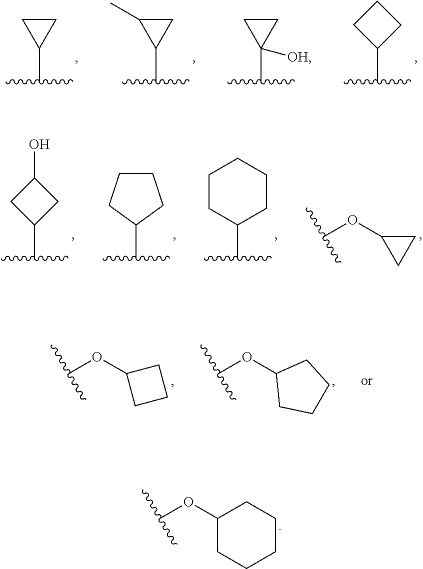

[44]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [43], wherein, R₃ is selected from —H, —F, —Cl, —CH₃, —CH(CH₃)₂, —CF₃, —S—CF₃ or

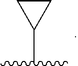 [SMILES: *C1CC1]

[45]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [44], wherein, R₃ is selected from —H.

[46]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [45], wherein, the moiety of —X₂—R₂ or —O—R₂ is selected from

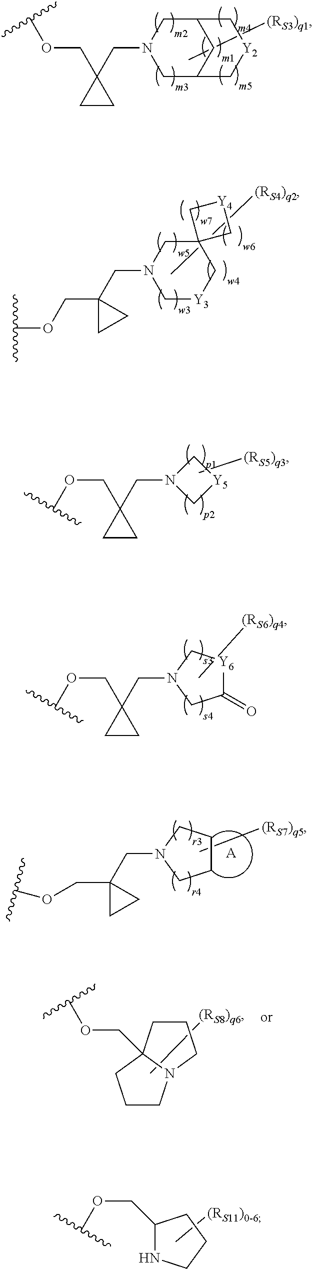

Y₂ is selected from O, S, SO, SO₂, C═O, NH or CH₂, when Y₂ is selected from NH or CH₂, the NH or CH₂ is optionally substituted with 1 or 2 R_{S3};
m₁, m₂, m₃, m₄ or m₅ is independently selected from 0, 1, 2, 3, 4, 5 or 6;
Y₃ and Y₄ are each independently selected from O, S, SO, SO₂, C═O, NH or CH₂, when Y₃ and Y₄ is selected from NH or CH₂, the NH or CH₂ is optionally substituted with 1 or 2 R_{S4};
w₃, w₄, w₅, w₆ and w₇ are each independently selected from 0, 1, 2, 3, 4, 5 or 6; provided that w₆ and w₇ are not 0 at the same time;
Y₅ is selected from O, S, SO, SO₂, C═O, NH or CH₂, when Y₅ is selected from NH or CH₂, the NH or CH₂ is optionally substituted with 1 or 2 R_{S5};
p₁ and p₂ are each independently selected from 0, 1, 2, 3, 4, 5, or 6, provided that p₁ and p₂ are not 0 at the same time;
Y₆ is selected from O, S, NH or CH₂, when Y₆ is selected from NH or CH₂, the NH or CH₂ is optionally substituted with 1 or 2 R_{S6};
s₃ and s₄ are each independently selected from 0, 1, 2, 3, 4, 5, or 6, provided that s₃ and s₄ are not 0 at the same time;
r₃ and r₄ are each independently selected from 0, 1, 2, 3, 4, 5, or 6;
ring A at each occurrence is independently selected from a 4 membered carbocyclic ring, a 5 membered carbocyclic ring, a 6 membered carbocyclic ring, a 4 membered heterocyclic ring including 1 ring member selected from N, a 5 membered heterocyclic ring including 1 to 2 ring members selected from N, or O, a 6 membered heterocyclic ring including 1 to 2 ring members selected from N, O or S, a phenyl ring, a 5 membered heteroaryl ring including 1 to 2 ring members selected from N, O, or S, or a 6 membered heteroaryl ring including 1 ring member selected from N.

[47]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [46], wherein:
Y₂ is selected from 0, C═O, NH or CH₂, when Y₂ is selected from NH or CH₂, the NH or CH₂ is optionally substituted with 1 or 2 R_{S3};
m₁ is selected from 0, 1, 2 or 3; m₂ is selected from 0, 1, 2 or 3; m₃ is selected from 0, 1, 2 or 3; m₄ is selected from 0, 1, 2 or 3; m₅ is selected from 0, 1, 2 or 3;
Y₃ and Y₄ are each independently selected from O, S, SO₂, NH or CH₂, when Y₃ and Y₄ is selected from NH or CH₂, the NH or CH₂ is optionally substituted with 1 or 2 R_{S4};
w₃ is selected from 0, 1, 2 or 3; w₄ is selected from 0, 1, 2 or 3; w₅ is selected from 0, 1, 2 or 3; w₆ is selected from 0, 1, 2 or 3; w₇ is selected from 0, 1, 2 or 3; provided that w₆ and w₇ is not 0 at the same time;
Y₅ is selected from O, S, SO₂, NH, CH₂ or C═O, when Y₅ is selected from NH or CH₂, the NH or CH₂ is optionally substituted with 1 or 2 R_{S5};
p₁ is selected from 1, 2, 3 or 4; p₂ is selected from 1, 2, 3 or 4;
Y₆ is selected from 0, S or NH, when Y₆ is selected from NH, the NH or CH₂ is optionally substituted with R_{S6};
s₁ is selected from 1, 2, 3 or 4; s₂ is selected from 1, 2, 3 or 4;
r₃ is selected from 1, 2 or 3; r₄ is selected from 1, 2 or 3;
ring A at each occurrence is independently selected from a 4 membered carbocyclic ring, a 5 membered carbocyclic ring, a 6 membered carbocyclic ring, a 4 membered heterocyclic ring including 1 ring member selected from N, a 5 membered heterocyclic ring including 1 to 2 ring members selected from N, or O, a 6 membered heterocyclic ring including 1 to 2 ring members selected from N, O or S, a phenyl ring, a 5 membered heteroaryl ring including 1 to 2 ring members selected from N, O, or S, or a 6 membered heteroaryl ring including 1 ring member selected from N.

[48]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [47], wherein:
Each of R_{S3} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂; preferably, each of R_{S3} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₁ is selected from 0, 1, or 2;
Each of R_{S4} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂; preferably, each of R_{S4} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₂ is selected from 0, 1, or 2;
Each of R_{S5} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S5} is independently selected from —F, —CH₃, —CH₂CH₃, —CF₃, —CN, oxo, —NH₂, —OH, —O—CH₃, —OCH₂—COO(CH₂CH₃), —COOH, —COO(CH₃), —CH₂OH, or —CH₂NH₂;
q₃ is selected from 0, 1, or 2;
Each of R_{S6} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S6} is independently selected from —F, —CH₃, —CH₂CH₃, —CF₃, —CN, oxo, —NH₂, —OH, —O—CH₃, —OCH₂—COO(CH₂CH₃), —COOH, —COO(CH₃), —CH₂OH, or —CH₂NH₂;
q₄ is selected from 0, 1, or 2; Each of R_{S7} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of Rₛ₇ is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₅ is selected from 0, 1, or 2;
Each of R_{S8} at each occurrence is independently selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-8 membered cycloalkyl, 4-8 membered heterocyclyl containing 1, 2 or 3 heteroatoms selected from N, O or S, phenyl or 5-6 membered heteroaryl containing 1, 2 or 3 heteroatoms selected from N, O or S, wherein said —C₁₋₆alkyl, haloC₁₋₆alkyl, haloC₁₋₆alkoxy, 3-8 membered cycloalkyl, 4-8 membered heterocyclyl, phenyl or 5-6 membered heteroaryl is optionally independently substituted with 1, 2 or 3 substituents selected from halogen, —C₁₋₆-alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-6 membered cycloalkyl, 4-6 membered heterocyclyl, phenyl or 5-6 membered heteroaryl;
Each of (R_{N1} or R_{N2}) in R_{S8} is independently selected from hydrogen or —C₁₋₆alkyl;
Optionally, (R_{N1} and R_{N2}) in R_{S8} together with the nitrogen atom to which they are both attached form a 3-6 membered heterocyclic ring which is optionally substituted with one or more substituents selected from halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
q₆ is selected from 0, 1, 2 or 3;
Each of R_{S11} is selected from —C₁₋₃alkyl.

[49]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [48], wherein, the moiety of —X₂—R₂ or —O—R₂ is selected from any one of the structures in the Table 14:

TABLE 14

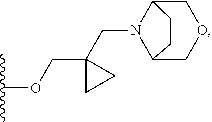 [SMILES: *OCC1(CN2C3CCC2COC3)CC1]

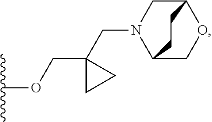 [SMILES: *OCC1(CN2C[C@H]3CC[C@@H]2CO3)CC1]

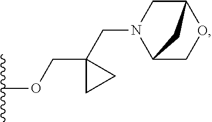 [SMILES: *OCC1(CN2C[C@H]3C[C@@H]2CO3)CC1]

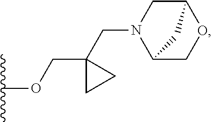 [SMILES: *OCC1(CN2C[C@@H]3C[C@H]2CO3)CC1]

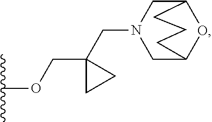 [SMILES: *OCC1(CN2CC3CCCC(C2)O3)CC1]

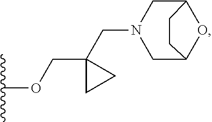 [SMILES: *OCC1(CN2CC3CCC(C2)O3)CC1]

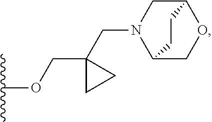 [SMILES: *OCC1(CN2C[C@@H]3CC[C@H]2CO3)CC1]

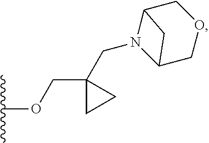 [SMILES: *OCC1(CN2C3COCC2C3)CC1]

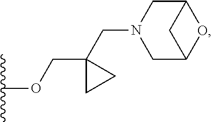 [SMILES: *OCC1(CN2CC3CC(C2)O3)CC1]

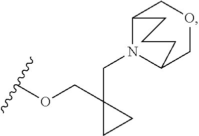 [SMILES: *OCC1(CN2C3CCCC2COC3)CC1]

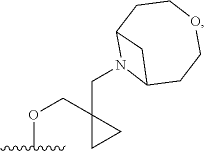 [SMILES: *OCC1(CN2C3CCOCCC2C3)CC1]

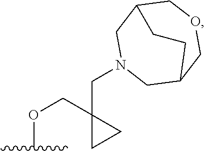 [SMILES: *OCC1(CN2CC3CCC(COC3)C2)CC1]

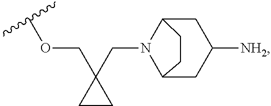 [SMILES: *OCC1(CN2C3CCC2CC(N)C3)CC1]

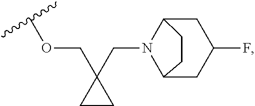 [SMILES: *OCC1(CN2C3CCC2CC(F)C3)CC1]

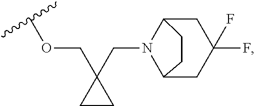 [SMILES: *OCC1(CN2C3CCC2CC(F)(F)C3)CC1]

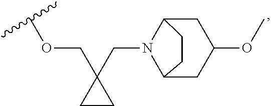 [SMILES: *OCC1(CN2C3CCC2CC(OC)C3)CC1]

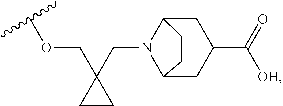 [SMILES: *OCC1(CN2C3CCC2CC(C(=O)O)C3)CC1]

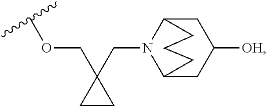 [SMILES: *OCC1(CN2C3CCCC2CC(O)C3)CC1]

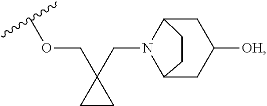 [SMILES: *OCC1(CN2C3CCC2CC(O)C3)CC1]

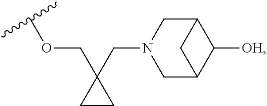 [SMILES: *OCC1(CN2CC3CC(C2)C3O)CC1]

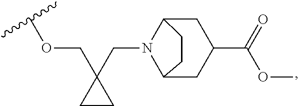

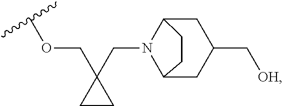 [SMILES: *OCC1(CN2C3CCC2CC(CO)C3)CC1]

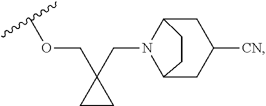 [SMILES: *OCC1(CN2C3CCC2CC(C#N)C3)CC1]

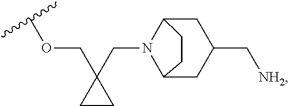 [SMILES: *OCC1(CN2C3CCC2CC(CN)C3)CC1]

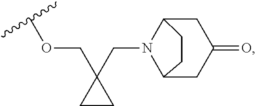 [SMILES: *OCC1(CN2C3CCC2CC(=O)C3)CC1]

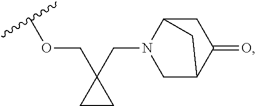 [SMILES: *OCC1(CN2CC3CC2CC3=O)CC1]

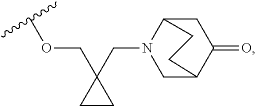 [SMILES: *OCC1(CN2CC3CCC2CC3=O)CC1]

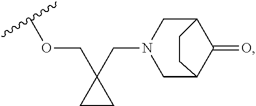 [SMILES: *OCC1(CN2CC3CCC(C2)C3=O)CC1]

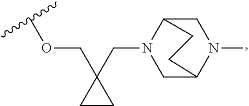

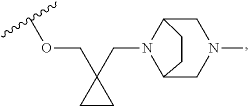

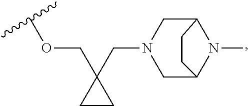

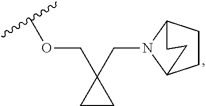 [SMILES: *OCC1(CN2C3CCC2CC3)CC1]

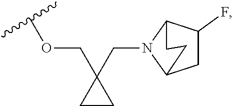 [SMILES: *OCC1(CN2C3CCC2C(F)C3)CC1]

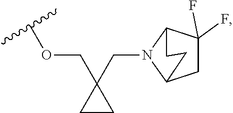 [SMILES: *OCC1(CN2C3CCC2C(F)(F)C3)CC1]

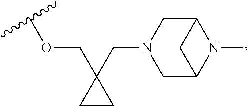

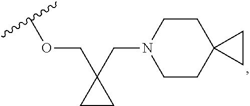 [SMILES: *OCC1(CN2CCC3(CC2)CC3)CC1]

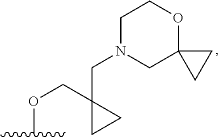 [SMILES: *OCC1(CN2CCOC3(CC3)C2)CC1]

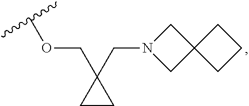 [SMILES: *OCC1(CN2CC3(CCC3)C2)CC1]

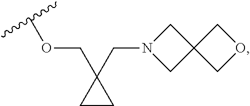 [SMILES: *OCC1(CN2CC3(COC3)C2)CC1]

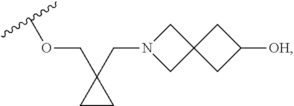 [SMILES: *OCC1(CN2CC3(CC(O)C3)C2)CC1]

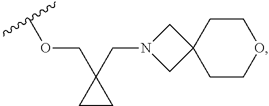 [SMILES: *OCC1(CN2CC3(CCOCC3)C2)CC1]

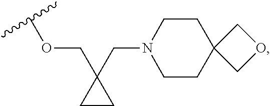 [SMILES: *OCC1(CN2CCC3(CC2)COC3)CC1]

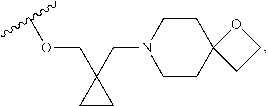 [SMILES: *OCC1(CN2CCC3(CCO3)CC2)CC1]

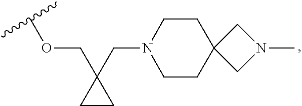 [SMILES: *OCC1(CN2CCC3(CC2)CN(C)C3)CC1]

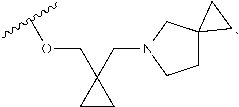 [SMILES: *OCC1(CN2CCC3(CC3)C2)CC1]

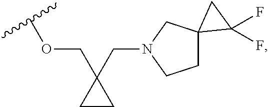 [SMILES: *OCC1(CN2CCC3(C2)CC3(F)F)CC1]

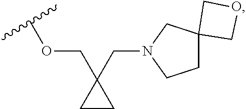 [SMILES: *OCC1(CN2CCC3(COC3)C2)CC1]

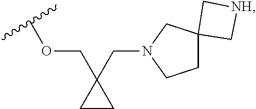 [SMILES: *OCC1(CN2CCC3(CNC3)C2)CC1]

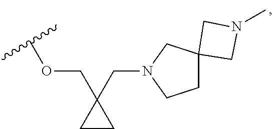 [SMILES: *OCC1(CN2CCC3(CN(C)C3)C2)CC1]

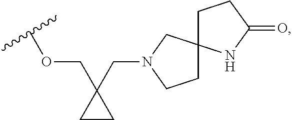 [SMILES: *OCC1(CN2CCC3(CCC(=O)N3)C2)CC1]

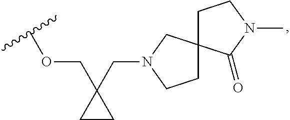 [SMILES: *OCC1(CN2CCC3(CCN(C)C3=O)C2)CC1]

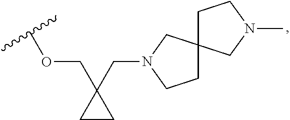 [SMILES: *OCC1(CN2CCC3(CCN(C)C3)C2)CC1]

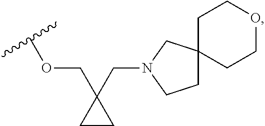 [SMILES: *OCC1(CN2CCC3(CCOCC3)C2)CC1]

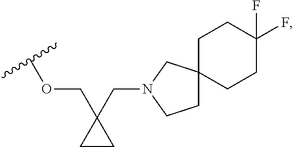 [SMILES: *OCC1(CN2CCC3(CCC(F)(F)CC3)C2)CC1]

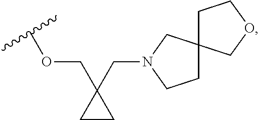 [SMILES: *OCC1(CN2CCC3(CCOC3)C2)CC1]

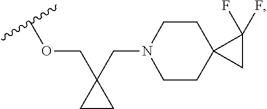 [SMILES: *OCC1(CN2CCC3(CC2)CC3(F)F)CC1]

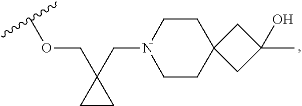 [SMILES: *OCC1(CN2CCC3(CC2)CC(C)(O)C3)CC1]

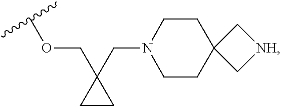 [SMILES: *OCC1(CN2CCC3(CC2)CNC3)CC1]

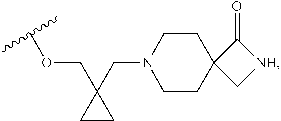 [SMILES: *OCC1(CN2CCC3(CC2)CNC3=O)CC1]

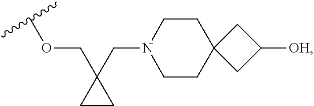 [SMILES: *OCC1(CN2CCC3(CC2)CC(O)C3)CC1]

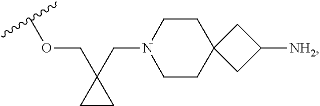 [SMILES: *OCC1(CN2CCC3(CC2)CC(N)C3)CC1]

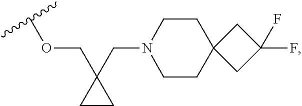 [SMILES: *OCC1(CN2CCC3(CC2)CC(F)(F)C3)CC1]

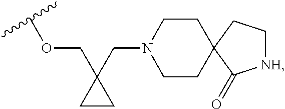 [SMILES: *OCC1(CN2CCC3(CCNC3=O)CC2)CC1]

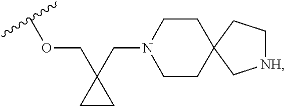 [SMILES: *OCC1(CN2CCC3(CCNC3)CC2)CC1]

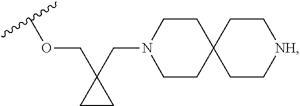 [SMILES: *OCC1(CN2CCC3(CCNCC3)CC2)CC1]

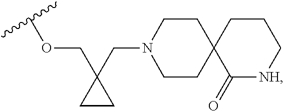 [SMILES: *OCC1(CN2CCC3(CCCNC3=O)CC2)CC1]

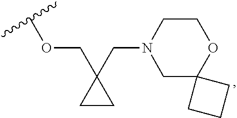 [SMILES: *OCC1(CN2CCOC3(CCC3)C2)CC1]

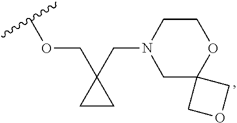 [SMILES: *OCC1(CN2CCOC3(COC3)C2)CC1]

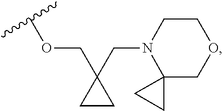 [SMILES: *OCC1(CN2CCOCC23CC3)CC1]

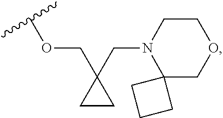 [SMILES: *OCC1(CN2CCOCC23CCC3)CC1]

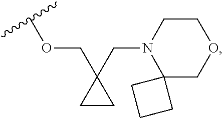 [SMILES: *OCC1(CN2CCOCC23CCC3)CC1]

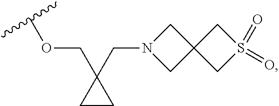 [SMILES: *OCC1(CN2CC3(C2)CS(=O)(=O)C3)CC1]

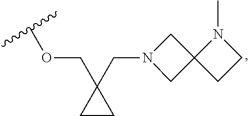 [SMILES: *OCC1(CN2CC3(CCN3C)C2)CC1]

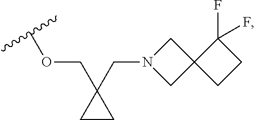 [SMILES: *OCC1(CN2CC3(CCC3(F)F)C2)CC1]

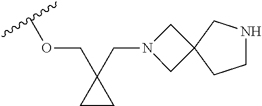 [SMILES: *OCC1(CN2CC3(CCNC3)C2)CC1]

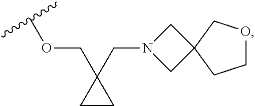 [SMILES: *OCC1(CN2CC3(CCOC3)C2)CC1]

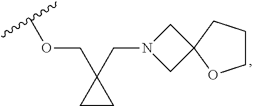 [SMILES: *OCC1(CN2CC3(CCCO3)C2)CC1]

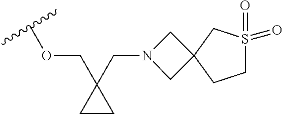 [SMILES: *OCC1(CN2CC3(CCS(=O)(=O)C3)C2)CC1]

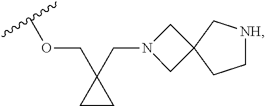 [SMILES: *OCC1(CN2CC3(CCNC3)C2)CC1]

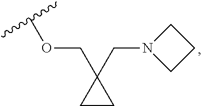 [SMILES: *OCC1(CN2CCC2)CC1]

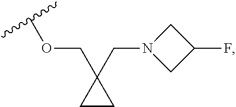 [SMILES: *OCC1(CN2CC(F)C2)CC1]

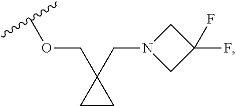 [SMILES: *OCC1(CN2CC(F)(F)C2)CC1]

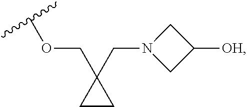 [SMILES: *OCC1(CN2CC(O)C2)CC1]

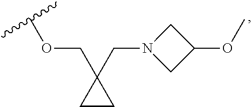 [SMILES: *OCC1(CN2CC(OC)C2)CC1]

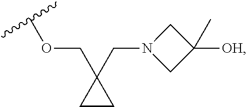 [SMILES: *OCC1(CN2CC(C)(O)C2)CC1]

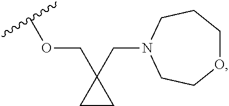 [SMILES: *OCC1(CN2CCCOCC2)CC1]

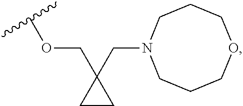 [SMILES: *OCC1(CN2CCCOCCC2)CC1]

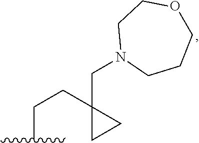 [SMILES: *CCC1(CN2CCCOCC2)CC1]

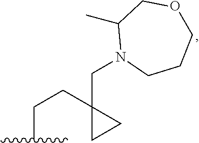 [SMILES: *CCC1(CN2CCCOCC2C)CC1]

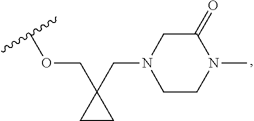 [SMILES: *OCC1(CN2CCN(C)C(=O)C2)CC1]

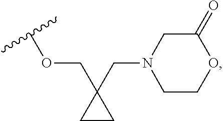 [SMILES: *OCC1(CN2CCOC(=O)C2)CC1]

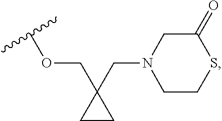 [SMILES: *OCC1(CN2CCSC(=O)C2)CC1]

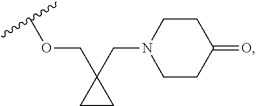 [SMILES: *OCC1(CN2CCC(=O)CC2)CC1]

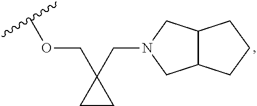 [SMILES: *OCC1(CN2CC3CCCC3C2)CC1]

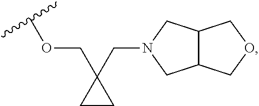 [SMILES: *OCC1(CN2CC3COCC3C2)CC1]

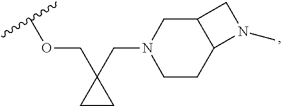 [SMILES: *OCC1(CN2CCC3C(C2)CN3C)CC1]

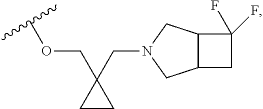 [SMILES: *OCC1(CN2CC3CC(F)(F)C3C2)CC1]

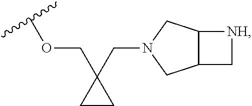 [SMILES: *OCC1(CN2CC3CNC3C2)CC1]

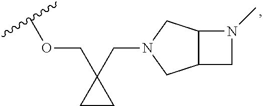 [SMILES: *OCC1(CN2CC3CN(C)C3C2)CC1]

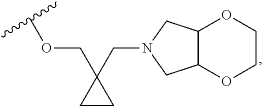 [SMILES: *OCC1(CN2CC3OCCOC3C2)CC1]

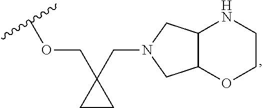 [SMILES: *OCC1(CN2CC3NCCOC3C2)CC1]

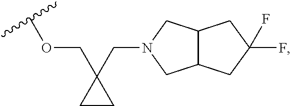 [SMILES: *OCC1(CN2CC3CC(F)(F)CC3C2)CC1]

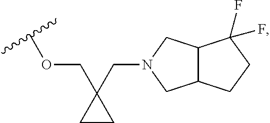 [SMILES: *OCC1(CN2CC3CCC(F)(F)C3C2)CC1]

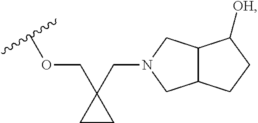 [SMILES: *OCC1(CN2CC3CCC(O)C3C2)CC1]

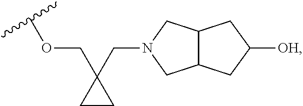 [SMILES: *OCC1(CN2CC3CC(O)CC3C2)CC1]

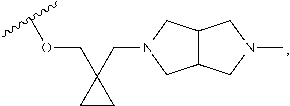 [SMILES: *OCC1(CN2CC3CN(C)CC3C2)CC1]

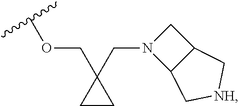 [SMILES: *OCC1(CN2CC3CNCC32)CC1]

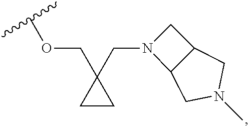 [SMILES: *OCC1(CN2CC3CN(C)CC32)CC1]

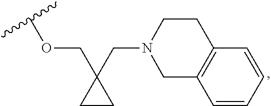 [SMILES: *OCC1(CN2CCc3ccccc3C2)CC1]

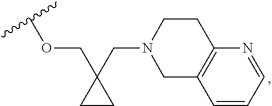 [SMILES: *OCC1(CN2CCc3ncccc3C2)CC1]

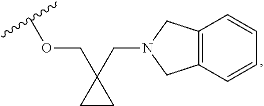 [SMILES: *OCC1(CN2Cc3ccccc3C2)CC1]

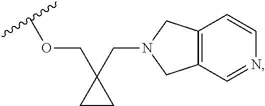 [SMILES: *OCC1(CN2Cc3ccncc3C2)CC1]

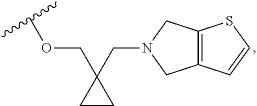 [SMILES: *OCC1(CN2Cc3ccsc3C2)CC1]

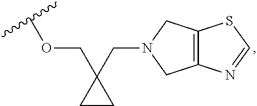 [SMILES: *OCC1(CN2Cc3ncsc3C2)CC1]

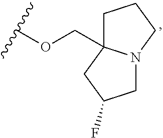 [SMILES: *OCC12CCCN1C[C@H](F)C2]

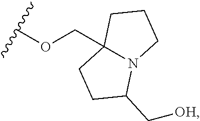 [SMILES: *OCC12CCCN1C(CO)CC2]

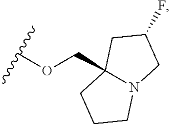 [SMILES: *OC[C@]12CCCN1C[C@@H](F)C2]

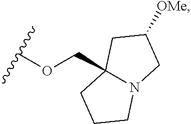 [SMILES: *OC[C@]12CCCN1C[C@@H](OC)C2]

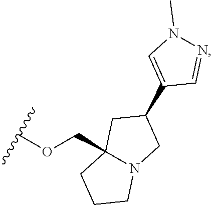 [SMILES: *OC[C@]12CCCN1C[C@H](c1cnn(C)c1)C2]

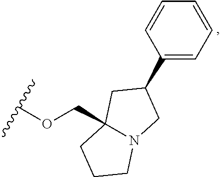 [SMILES: *OC[C@]12CCCN1C[C@H](c1ccccc1)C2]

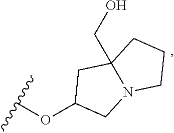 [SMILES: *OC1CN2CCCC2(CO)C1]

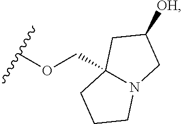 [SMILES: *OC[C@@]12CCCN1C[C@H](O)C2]

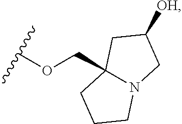 [SMILES: *OC[C@]12CCCN1C[C@H](O)C2]

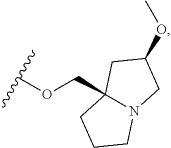 [SMILES: *OC[C@]12CCCN1C[C@H](OC)C2]

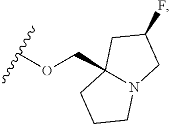 [SMILES: *OC[C@]12CCCN1C[C@H](F)C2]

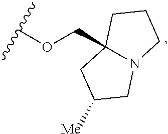 [SMILES: *OC[C@@]12CCCN1C[C@H](C)C2]

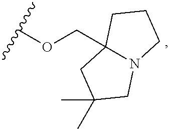 [SMILES: *OCC12CCCN1CC(C)(C)C2]

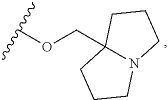 [SMILES: *OCC12CCCN1CCC2]

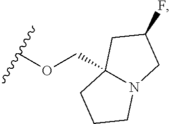 [SMILES: *OC[C@@]12CCCN1C[C@H](F)C2]

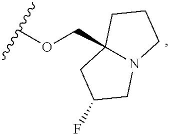 [SMILES: *OC[C@@]12CCCN1C[C@H](F)C2]

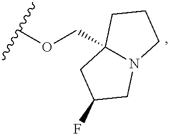 [SMILES: *OC[C@]12CCCN1C[C@@H](F)C2]

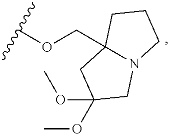 [SMILES: *OCC12CCCN1CC(OC)(OC)C2]

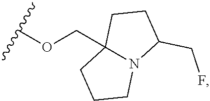 [SMILES: *OCC12CCCN1C(CF)CC2]

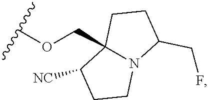 [SMILES: *OC[C@@]12CCC(CF)N1CC[C@@H]2C#N]

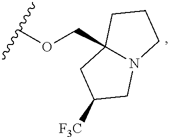 [SMILES: *OC[C@@]12CCCN1C[C@@H](C(F)(F)F)C2]

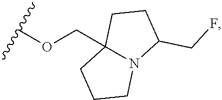 [SMILES: *OCC12CCCN1C(CF)CC2]

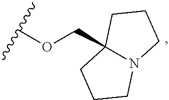

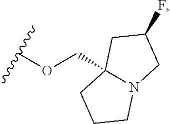 [SMILES: *OC[C@@]12CCCN1C[C@H](F)C2]

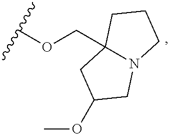 [SMILES: *OCC12CCCN1CC(OC)C2]

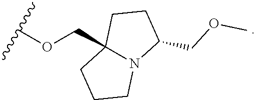 [SMILES: *OC[C@]12CCCN1[C@@H](COC)CC2]

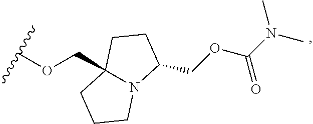 [SMILES: *OC[C@]12CCCN1[C@@H](COC(=O)N(C)C)CC2]

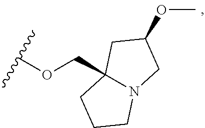 [SMILES: *OC[C@]12CCCN1C[C@H](OC)C2]

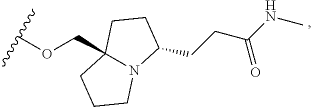 [SMILES: *OC[C@]12CCCN1[C@@H](CCC(=O)NC)CC2]

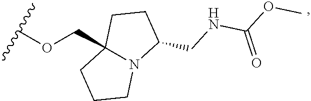 [SMILES: *OC[C@]12CCCN1[C@@H](CNC(=O)OC)CC2]

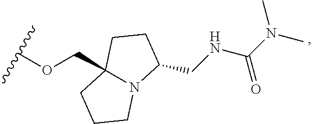 [SMILES: *OC[C@]12CCCN1[C@@H](CNC(=O)N(C)C)CC2]

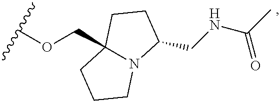 [SMILES: *OC[C@]12CCCN1[C@@H](CNC(C)=O)CC2]

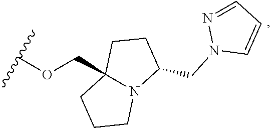 [SMILES: *OC[C@]12CCCN1[C@@H](Cn1cccn1)CC2]

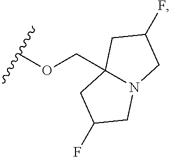 [SMILES: *OCC12CC(F)CN1CC(F)C2]

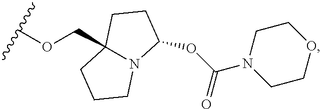 [SMILES: *OC[C@]12CCCN1[C@@H](OC(=O)N1CCOCC1)CC2]

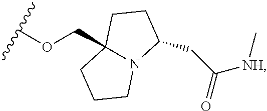 [SMILES: *OC[C@]12CCCN1[C@@H](CC(=O)NC)CC2]

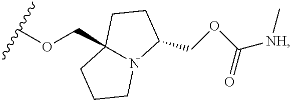 [SMILES: *OC[C@]12CCCN1[C@@H](COC(=O)NC)CC2]

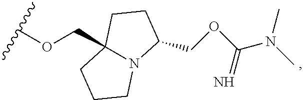 [SMILES: *OC[C@]12CCCN1[C@@H](COC(=N)N(C)C)CC2]

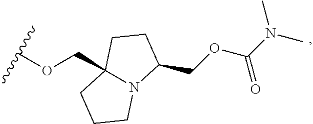 [SMILES: *OC[C@]12CCCN1[C@H](COC(=O)N(C)C)CC2]

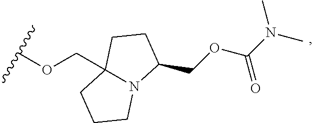 [SMILES: *OCC12CCCN1[C@H](COC(=O)N(C)C)CC2]

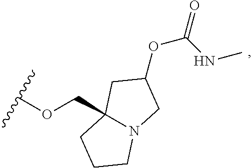 [SMILES: *OC[C@]12CCCN1CC(OC(=O)NC)C2]

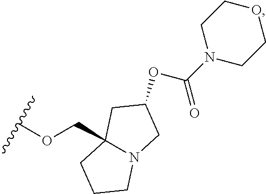 [SMILES: *OC[C@]12CCCN1C[C@@H](OC(=O)N1CCOCC1)C2]

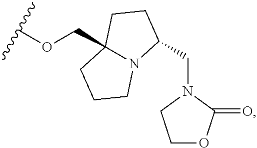 [SMILES: *OC[C@]12CCCN1[C@@H](CN1CCOC1=O)CC2]

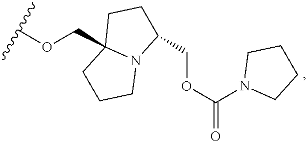 [SMILES: *OC[C@]12CCCN1[C@@H](COC(=O)N1CCCC1)CC2]

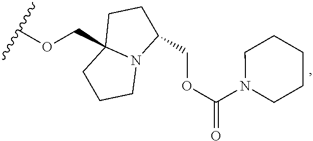 [SMILES: *OC[C@]12CCCN1[C@@H](COC(=O)N1CCCCC1)CC2]

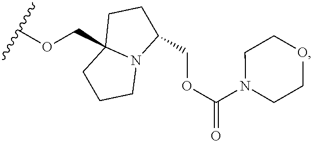 [SMILES: *OC[C@]12CCCN1[C@@H](COC(=O)N1CCOCC1)CC2]

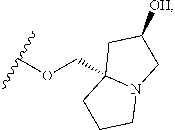 [SMILES: *OC[C@@]12CCCN1C[C@H](O)C2]

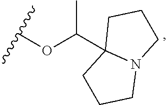 [SMILES: *OC(C)C12CCCN1CCC2]

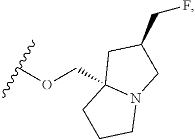 [SMILES: *OC[C@@]12CCCN1C[C@H](CF)C2]

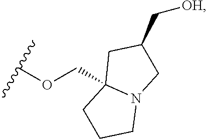 [SMILES: *OC[C@@]12CCCN1C[C@H](CO)C2]

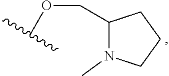 [SMILES: *OCC1CCCN1C]

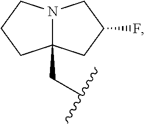 [SMILES: *C[C@@]12CCCN1C[C@H](F)C2]

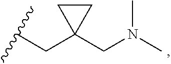 [SMILES: *CC1(CN(C)C)CC1]

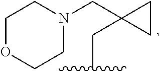 [SMILES: *CC1(CN2CCOCC2)CC1]

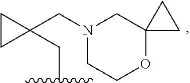 [SMILES: *CC1(CN2CCOC3(CC3)C2)CC1]

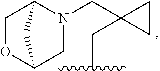 [SMILES: *CC1(CN2C[C@@H]3C[C@H]2CO3)CC1]

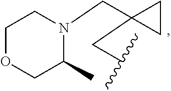 [SMILES: *CC1(CN2CCOC[C@@H]2C)CC1]

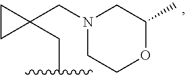 [SMILES: *CC1(CN2CCO[C@@H](C)C2)CC1]

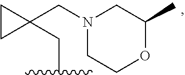 [SMILES: *CC1(CN2CCO[C@H](C)C2)CC1]

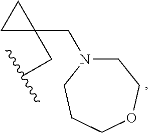 [SMILES: *CC1(CN2CCCOCC2)CC1]

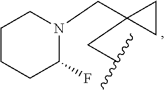 [SMILES: *CC1(CN2CCCC[C@H]2F)CC1]

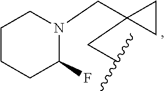 [SMILES: *CC1(CN2CCCC[C@@H]2F)CC1]

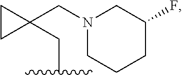 [SMILES: *CC1(CN2CCC[C@@H](F)C2)CC1]

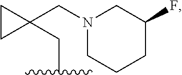 [SMILES: *CC1(CN2CCC[C@H](F)C2)CC1]

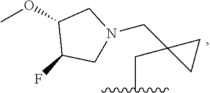 [SMILES: *CC1(CN2C[C@@H](F)[C@H](OC)C2)CC1]

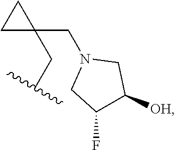 [SMILES: *CC1(CN2C[C@@H](O)[C@H](F)C2)CC1]

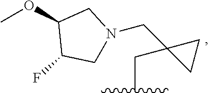 [SMILES: *CC1(CN2C[C@H](OC)[C@@H](F)C2)CC1]

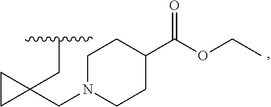 [SMILES: *CC1(CN2CCC(C(=O)OCC)CC2)CC1]

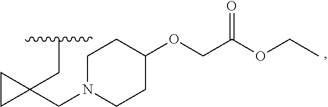 [SMILES: *CC1(CN2CCC(OCC(=O)OCC)CC2)CC1]

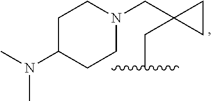 [SMILES: *CC1(CN2CCC(N(C)C)CC2)CC1]

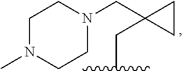 [SMILES: *CC1(CN2CCN(C)CC2)CC1]

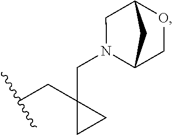 [SMILES: *CC1(CN2C[C@H]3C[C@@H]2CO3)CC1]

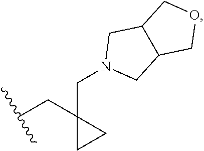 [SMILES: *CC1(CN2CC3COCC3C2)CC1]

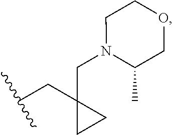 [SMILES: *CC1(CN2CCOC[C@@H]2C)CC1]

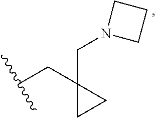 [SMILES: *CC1(CN2CCC2)CC1]

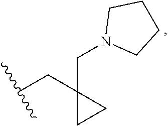 [SMILES: *CC1(CN2CCCC2)CC1]

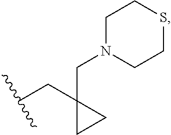 [SMILES: *CC1(CN2CCSCC2)CC1]

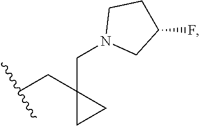 [SMILES: *CC1(CN2CC[C@H](F)C2)CC1]

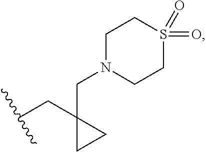 [SMILES: *CC1(CN2CCS(=O)(=O)CC2)CC1]

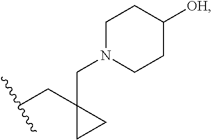 [SMILES: *CC1(CN2CCC(O)CC2)CC1]

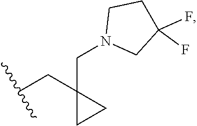 [SMILES: *CC1(CN2CCC(F)(F)C2)CC1]

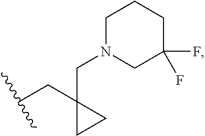 [SMILES: *CC1(CN2CCCC(F)(F)C2)CC1]

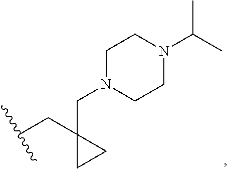 [SMILES: *CC1(CN2CCN(C(C)C)CC2)CC1]

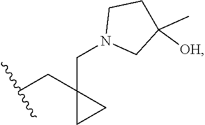 [SMILES: *CC1(CN2CCC(C)(O)C2)CC1]

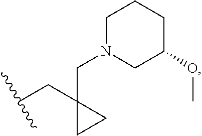 [SMILES: *CC1(CN2CCC[C@H](OC)C2)CC1]

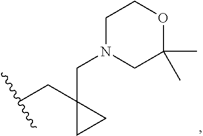 [SMILES: *CC1(CN2CCOC(C)(C)C2)CC1]

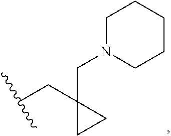 [SMILES: *CC1(CN2CCCCC2)CC1]

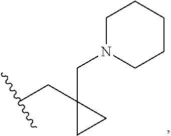 [SMILES: *CC1(CN2CCCCC2)CC1]

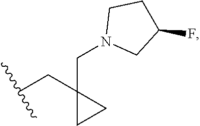 [SMILES: *CC1(CN2CC[C@@H](F)C2)CC1]

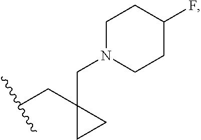 [SMILES: *CC1(CN2CCC(F)CC2)CC1]

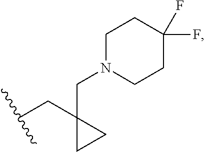 [SMILES: *CC1(CN2CCC(F)(F)CC2)CC1]

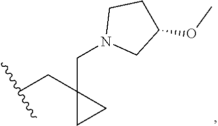 [SMILES: *CC1(CN2CC[C@H](OC)C2)CC1]

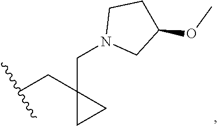 [SMILES: *CC1(CN2CC[C@@H](OC)C2)CC1]

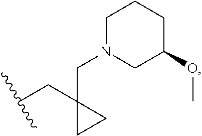 [SMILES: *CC1(CN2CCC[C@@H](OC)C2)CC1]

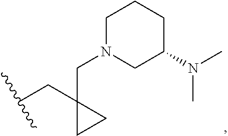 [SMILES: *CC1(CN2CCC[C@H](N(C)C)C2)CC1]

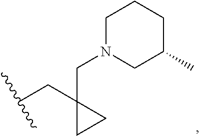 [SMILES: *CC1(CN2CCC[C@H](C)C2)CC1]

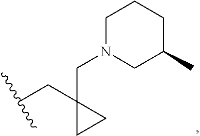 [SMILES: *CC1(CN2CCC[C@@H](C)C2)CC1]

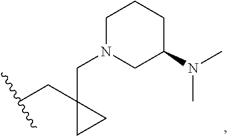 [SMILES: *CC1(CN2CCC[C@@H](N(C)C)C2)CC1]

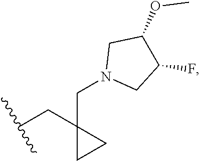 [SMILES: *CC1(CN2C[C@@H](F)[C@@H](OC)C2)CC1]

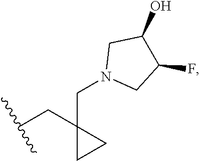 [SMILES: *CC1(CN2C[C@@H](O)[C@@H](F)C2)CC1]

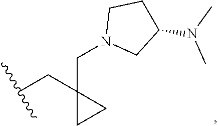 [SMILES: *CC1(CN2CC[C@H](N(C)C)C2)CC1]

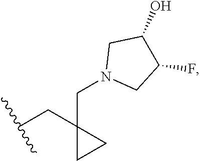 [SMILES: *CC1(CN2C[C@@H](F)[C@@H](O)C2)CC1]

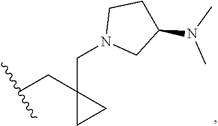 [SMILES: *CC1(CN2CC[C@@H](N(C)C)C2)CC1]

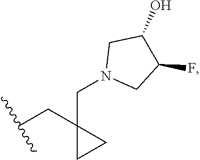 [SMILES: *CC1(CN2C[C@H](O)[C@@H](F)C2)CC1]

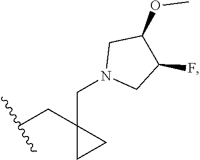 [SMILES: *CC1(CN2C[C@H](F)[C@H](OC)C2)CC1]

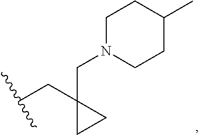 [SMILES: *CC1(CN2CCC(C)CC2)CC1]

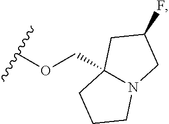 [SMILES: *OC[C@@]12CCCN1C[C@H](F)C2]

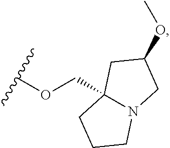 [SMILES: *OC[C@@]12CCCN1C[C@H](OC)C2]

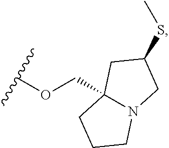 [SMILES: *OC[C@@]12CCCN1C[C@H](SC)C2]

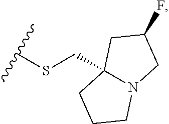 [SMILES: *SC[C@@]12CCCN1C[C@H](F)C2]

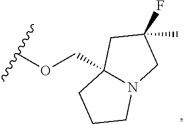 [SMILES: *OC[C@@]12CCCN1C[C@](C)(F)C2]

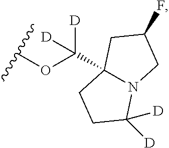 [SMILES: *OC([2H])([2H])[C@@]12CCC([2H])([2H])N1C[C@H](F)C2]

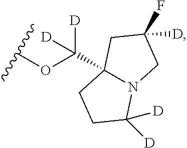

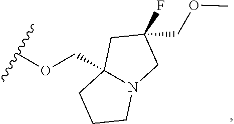 [SMILES: *OC[C@@]12CCCN1C[C@@](F)(COC)C2]

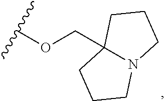 [SMILES: *OCC12CCCN1CCC2]

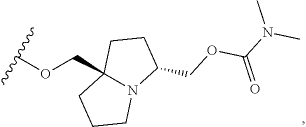 [SMILES: *OC[C@]12CCCN1[C@@H](COC(=O)N(C)C)CC2]

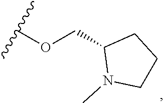 [SMILES: *OC[C@@H]1CCCN1C]

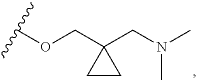 [SMILES: *OCC1(CN(C)C)CC1]

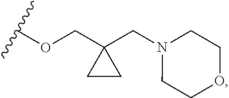 [SMILES: *OCC1(CN2CCOCC2)CC1]

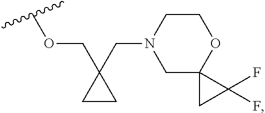 [SMILES: *OCC1(CN2CCOC3(C2)CC3(F)F)CC1]

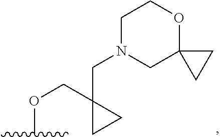 [SMILES: *OCC1(CN2CCOC3(CC3)C2)CC1]

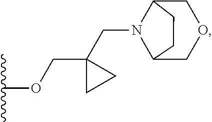 [SMILES: *OCC1(CN2C3CCC2COC3)CC1]

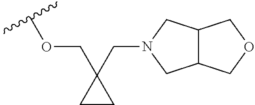 [SMILES: *OCC1(CN2CC3COCC3C2)CC1]

or

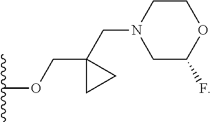 [SMILES: *OCC1(CN2CCO[C@H](F)C2)CC1]

[50]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [49], wherein, the moiety of —X₂—R₂ or —O—R₂ is selected from

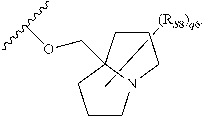

[51]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [50], wherein, each of R_{S8} at each occurrence is independently selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-8 membered cycloalkyl, 4-8 membered heterocyclyl containing 1, 2 or 3 heteroatoms selected from N, O or S, phenyl or 5-6 membered heteroaryl containing 1, 2 or 3 heteroatoms selected from N, O or S, wherein said —C₁₋₆alkyl, haloC₁₋₆alkyl, haloC₁₋₆alkoxy, 3-8 membered cycloalkyl, 4-8 membered heterocyclyl, phenyl or 5-6 membered heteroaryl is optionally independently substituted with 1, 2 or 3 substituents selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(O)NR_{N1}R_{N2}, —NR_{N1}C(O)NR_{N1}R_{N2}, 3-6 membered cycloalkyl, 4-6 membered heterocyclyl, phenyl or 5-6 membered heteroaryl;
Each of (R_{N1} or R_{N2}) in R_{S8} is independently selected from hydrogen or —C₁₋₆alkyl;
Optionally, (R_{N1} and R_{N2}) in R_{S8} together with the nitrogen atom to which they are both attached form a 3-6 membered heterocyclic ring which is optionally substituted with one or more substituents selected from halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
q₆ is selected from 0, 1, 2 or 3.

[52]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [51], wherein, each of R_{S8} at each occurrence is independently selected from —F; methyl; —CF₃; —CN; oxo; —OH; —NH₂; —OCH₃; —NHC(═O)CH₃; —NHC(═O)OCH₃; —OC(═O)N(CH₃)₂; —NHC(═O)(CH₃)₂;

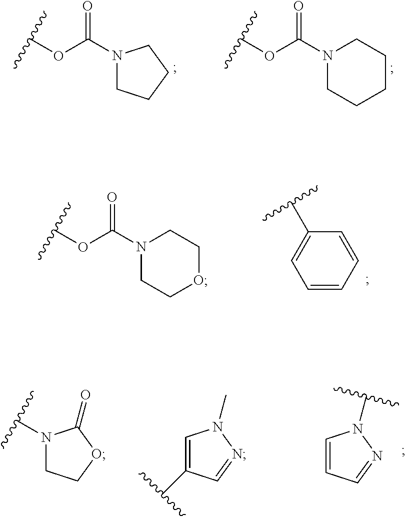

or methyl substituted with —F, —Cl, methyl, —CF₃, —CN, oxo, —OH, —NH₂, —OCH₃, —NHC(═O)CH₃, —NHC(═O)OCH₃, —OC(═O)N(CH₃)₂, —NHC(═O)N(CH₃)₂,

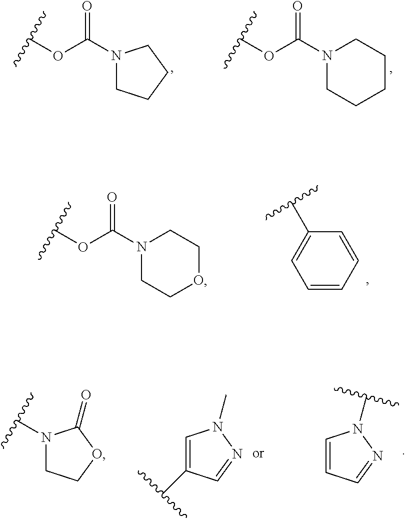

[53]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [52], wherein, each of R_{S8} at each occurrence is independently selected from —F, methyl, —CF₃, —CN, oxo, —OH, —NH₂, —OCH₃, —NHC(═O)CH₃, —NHC(═O)OCH₃, —OC(═O)N(CH₃)₂, —NHC(═O)N(CH₃)₂,

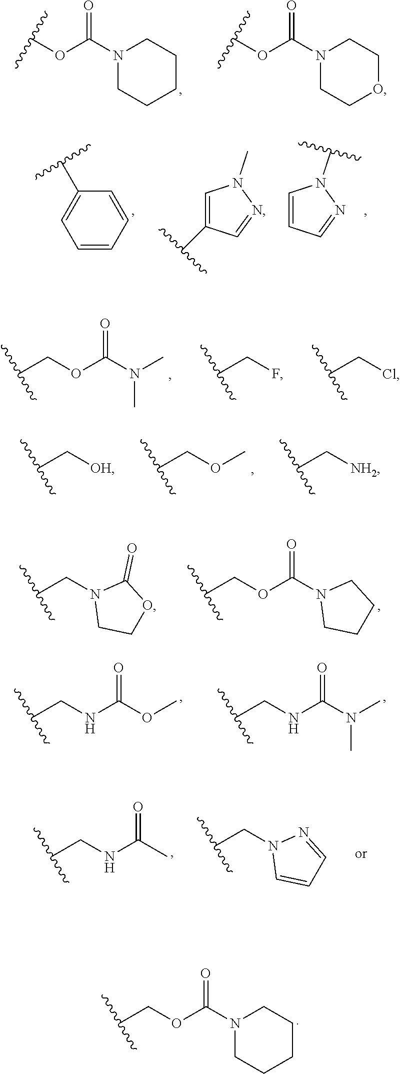

[54]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [53], wherein, the moiety of —X₂—R₂ or —O—R₂ is selected from any one in the Table 15:

TABLE 15

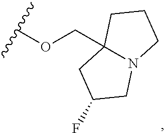 [SMILES: *OCC12CCCN1C[C@H](F)C2]

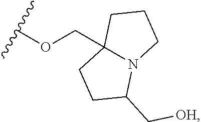 [SMILES: *OCC12CCCN1C(CO)CC2]

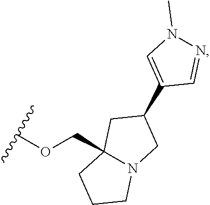 [SMILES: *OC[C@]12CCCN1C[C@H](c1cnn(C)c1)C2]

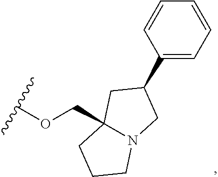 [SMILES: *OC[C@]12CCCN1C[C@H](c1ccccc1)C2]

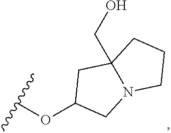 [SMILES: *OC1CN2CCCC2(CO)C1]

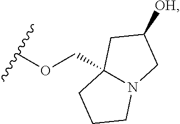 [SMILES: *OC[C@@]12CCCN1C[C@H](O)C2]

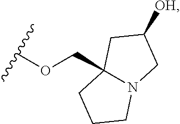 [SMILES: *OC[C@]12CCCN1C[C@H](O)C2]

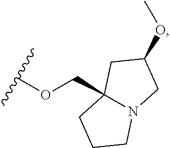 [SMILES: *OC[C@]12CCCN1C[C@H](OC)C2]

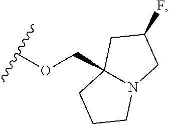 [SMILES: *OC[C@]12CCCN1C[C@H](F)C2]

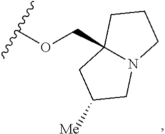 [SMILES: *OC[C@@]12CCCN1C[C@H](C)C2]

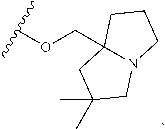 [SMILES: *OCC12CCCN1CC(C)(C)C2]

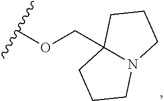 [SMILES: *OCC12CCCN1CCC2]

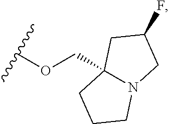 [SMILES: *OC[C@@]12CCCN1C[C@H](F)C2]

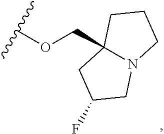 [SMILES: *OC[C@@]12CCCN1C[C@H](F)C2]

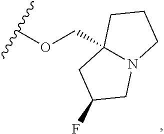 [SMILES: *OC[C@]12CCCN1C[C@@H](F)C2]

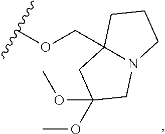 [SMILES: *OCC12CCCN1CC(OC)(OC)C2]

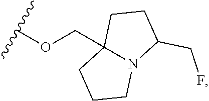 [SMILES: *OCC12CCCN1C(CF)CC2]

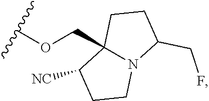 [SMILES: *OC[C@@]12CCC(CF)N1CC[C@@H]2C#N]

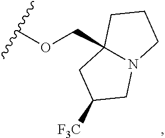 [SMILES: *OC[C@@]12CCCN1C[C@@H](C(F)(F)F)C2]

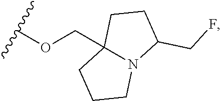 [SMILES: *OCC12CCCN1C(CF)CC2]

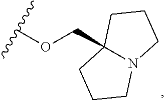

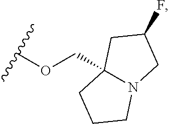 [SMILES: *OC[C@@]12CCCN1C[C@H](F)C2]

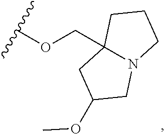 [SMILES: *OCC12CCCN1CC(OC)C2]

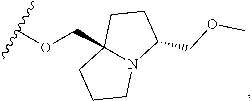 [SMILES: *OC[C@]12CCCN1[C@@H](COC)CC2]

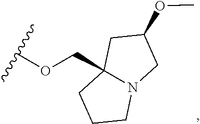 [SMILES: *OC[C@]12CCCN1C[C@H](OC)C2]

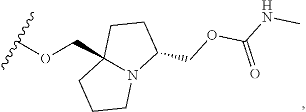 [SMILES: *OC[C@]12CCCN1[C@@H](COC(=O)NC)CC2]

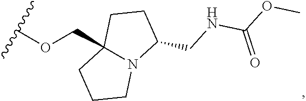 [SMILES: *OC[C@]12CCCN1[C@@H](CNC(=O)OC)CC2]

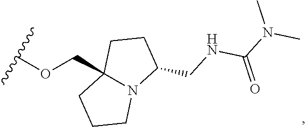 [SMILES: *OC[C@]12CCCN1[C@@H](CNC(=O)N(C)C)CC2]

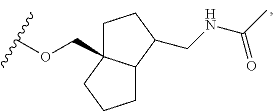 [SMILES: *OC[C@]12CCCC1C(CNC(C)=O)CC2]

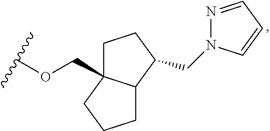 [SMILES: *OC[C@]12CCCC1[C@@H](Cn1cccn1)CC2]

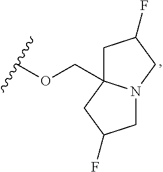 [SMILES: *OCC12CC(F)CN1CC(F)C2]

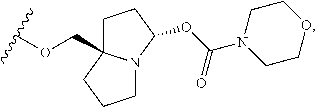 [SMILES: *OC[C@]12CCCN1[C@@H](OC(=O)N1CCOCC1)CC2]

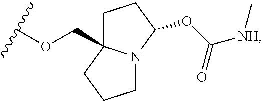 [SMILES: *OC[C@]12CCCN1[C@@H](OC(=O)NC)CC2]

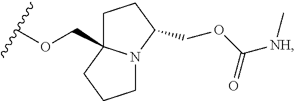 [SMILES: *OC[C@]12CCCN1[C@@H](COC(=O)NC)CC2]

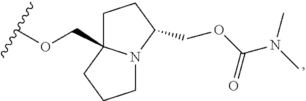 [SMILES: *OC[C@]12CCCN1[C@@H](COC(=O)N(C)C)CC2]

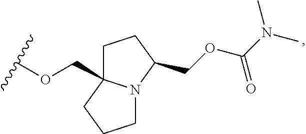 [SMILES: *OC[C@]12CCCN1[C@H](COC(=O)N(C)C)CC2]

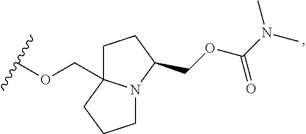 [SMILES: *OCC12CCCN1[C@H](COC(=O)N(C)C)CC2]

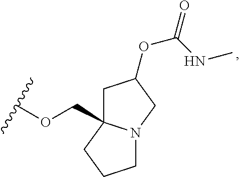 [SMILES: *OC[C@]12CCCN1CC(OC(=O)NC)C2]

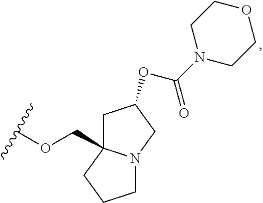 [SMILES: *OC[C@]12CCCN1C[C@@H](OC(=O)N1CCOCC1)C2]

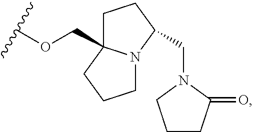 [SMILES: *OC[C@]12CCCN1[C@@H](CN1CCCC1=O)CC2]

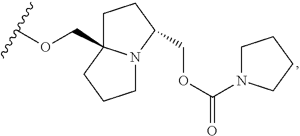 [SMILES: *OC[C@]12CCCN1[C@@H](COC(=O)N1CCCC1)CC2]

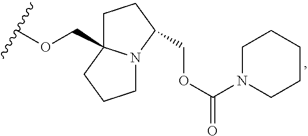 [SMILES: *OC[C@]12CCCN1[C@@H](COC(=O)N1CCCCC1)CC2]

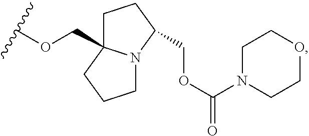 [SMILES: *OC[C@]12CCCN1[C@@H](COC(=O)N1CCOCC1)CC2]

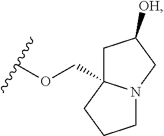 [SMILES: *OC[C@@]12CCCN1C[C@H](O)C2]

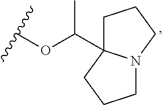 [SMILES: *OC(C)C12CCCN1CCC2]

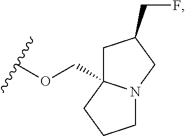 [SMILES: *OC[C@@]12CCCN1C[C@H](CF)C2]

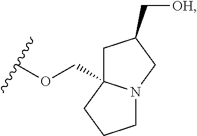 [SMILES: *OC[C@@]12CCCN1C[C@H](CO)C2]

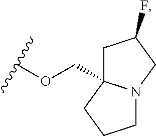 [SMILES: *OC[C@@]12CCCN1C[C@H](F)C2]

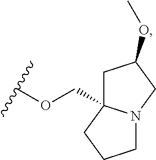 [SMILES: *OC[C@@]12CCCN1C[C@H](OC)C2]

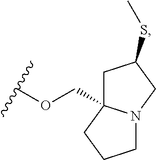 [SMILES: *OC[C@@]12CCCN1C[C@H](SC)C2]

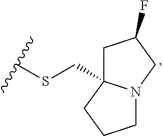 [SMILES: *SC[C@@]12CCCN1C[C@H](F)C2]

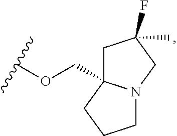 [SMILES: *OC[C@@]12CCCN1C[C@](C)(F)C2]

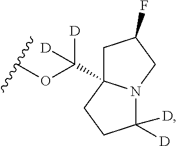

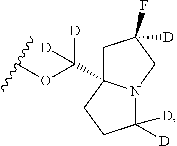

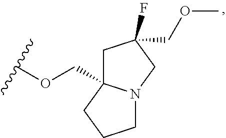 [SMILES: *OC[C@@]12CCCN1C[C@@](F)(COC)C2]

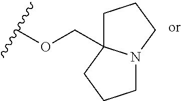 [SMILES: *OCC12CCCN1CCC2]

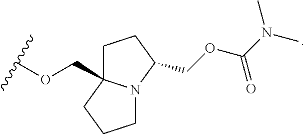 [SMILES: *OC[C@]12CCCN1[C@@H](COC(=O)N(C)C)CC2]

[55]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [54], wherein, the moiety of —X₂—R₂ or —O—R₂ is selected from

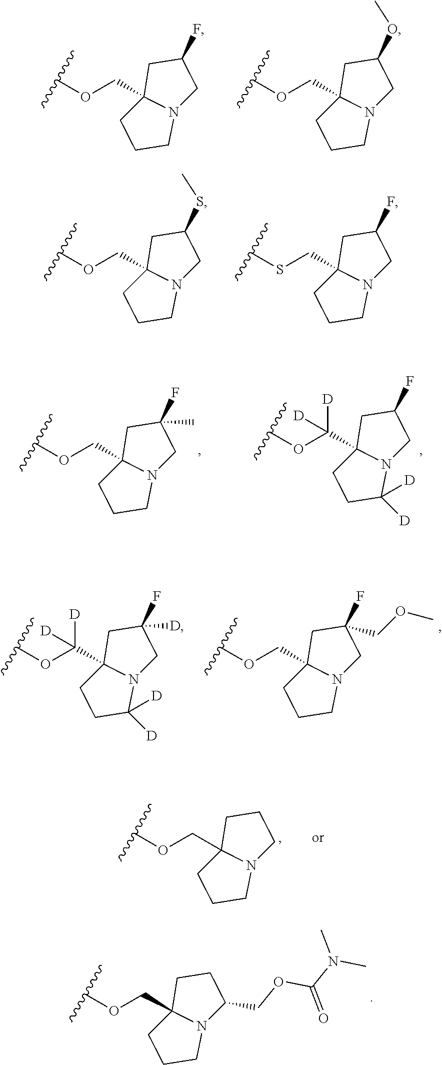

[56]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [55], wherein, the moiety of —X₂—R₂ or —O—R₂ is selected from

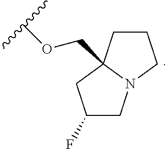 [SMILES: *OC[C@@]12CCCN1C[C@H](F)C2]

[57]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [56], wherein, R₄ is selected from phenyl, naphthyl, 5-10 membered heteroaryl or

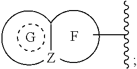

Said 5-10 membered heteroaryl is selected from 5 membered heteroaryl, 6 membered heteroaryl or

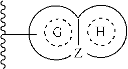

(the sum of ring members on Ring G and Ring H is selected from 9 or 10);
Z in the

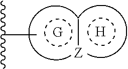

at each occurrence is independently selected from C;
Ring G in

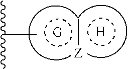

at each occurrence is independently selected from a phenyl or a 4-6 membered heteroaryl ring;

Ring H in

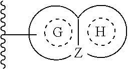

at each occurrence is independently selected from a phenyl or a 4-6 membered heteroaryl ring;

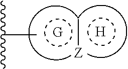

is optionally independently substituted with one or more R₄ₐ;
said heteroaryl ring containing 1, 2 or 3 ring members selected from N, O or S.

[58]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [57], wherein, R₄ is selected from

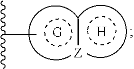

Ring G at each occurrence is independently selected from 6 membered aryl ring; 5 membered heteroaryl ring containing 1, 2 or 3 ring members selected from N, O or S; or 6 membered heteroaryl ring containing 1, 2 or 3 ring members selected from N, O or S; preferably, Ring G at each occurrence is independently selected from phenyl; 5 membered heteroaryl ring containing 1 ring member selected from N or S; or 6 membered heteroaryl ring containing 1 ring member selected from N;
Ring H at each occurrence is independently selected from 6 membered aryl ring; 5 membered heteroaryl ring containing 1, 2 or 3 ring members selected from N, O or S; or 6 membered heteroaryl ring containing 1, 2 or 3 ring members selected from N, O or S; preferably, Ring H at each occurrence is independently selected from phenyl or 5 membered heteroaryl ring containing 2 ring members selected from N.

[59]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [58], wherein, R₄ is selected from

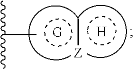

Ring G at each occurrence is independently selected from 5 membered heteroaryl ring containing 1 ring member selected from N or S; and Ring H at each occurrence is independently selected from phenyl;
Ring G at each occurrence is independently selected from 6 membered heteroaryl ring containing 1 ring member selected from N; and Ring H at each occurrence is independently selected from phenyl; or
Ring G at each occurrence is independently selected from phenyl; and Ring H at each occurrence is independently selected from 5 membered heteroaryl ring containing 2 ring members selected from N.

[60]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [59], wherein, R₄ is selected from any moiety in the Table 16:

TABLE 16

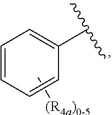

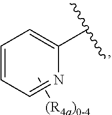

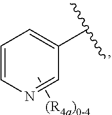

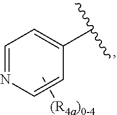

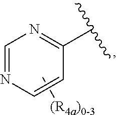

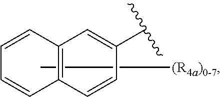

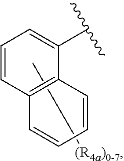

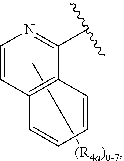

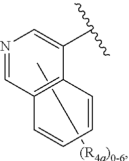

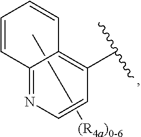

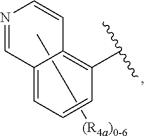

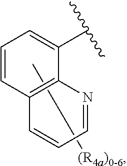

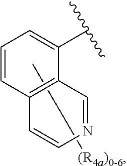

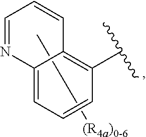

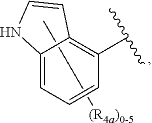

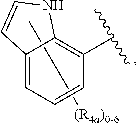

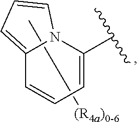

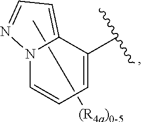

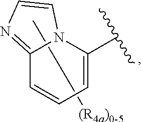

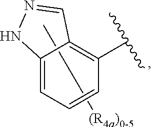

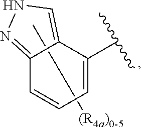

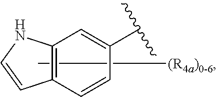

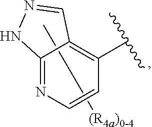

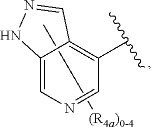

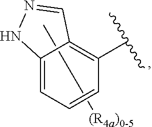

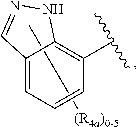

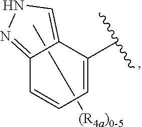

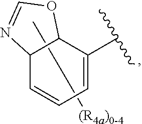

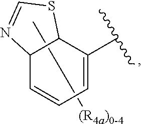

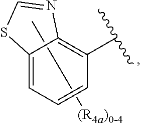

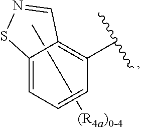

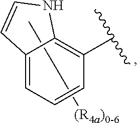

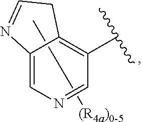

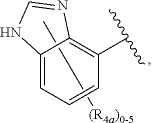

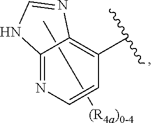

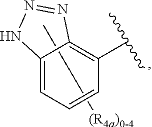

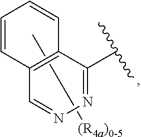

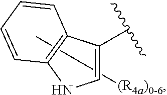

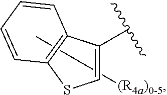

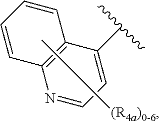

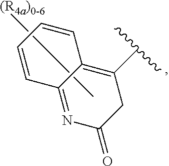

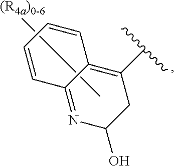

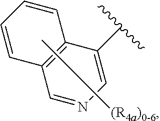

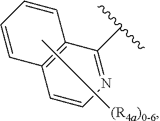

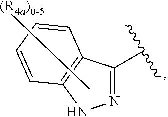

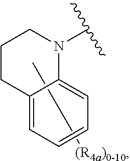

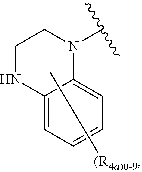

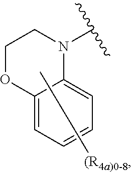

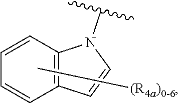

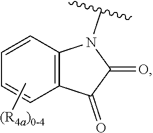

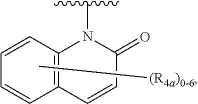

or

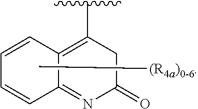

[61]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [60], wherein, R₄ is selected from

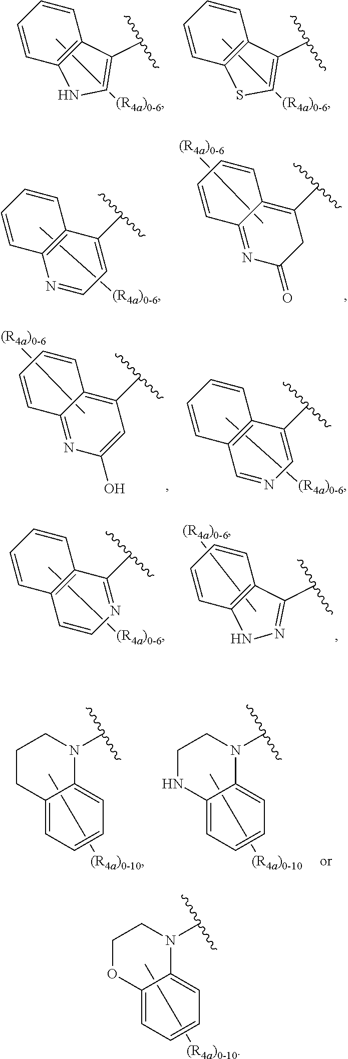

[62]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [61], wherein,
Each of R₄ₐ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NO₂, —NH(C₁₋₃alkyl), —N(C₁₋₃alkyl)₂, —OH, —O(C₁₋₃alkyl), —SH, —S(C₁₋₃alkyl), —C(═O)H, —C(═O)(C₁₋₃alkyl), —C(═O)OH, —C(═O)O(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, 3-6 membered cycloalkyl or 3-6 membered heterocyclyl is independently optionally substituted with 1, 2 or 3 R_{4b};
Each of R_{4b} is independently selected from —F; —C₁₋₃alkyl; haloC₁₋₃alkyl; —CN; —OH; —NH₂; —NH(C₁₋₃alkyl); —N(C₁₋₃alkyl)₂; —OC₁₋₃alkyl; or —C₁₋₃alkyl substituted with 1, 2 or 3 substituents selected from —F, haloC₁₋₃alkyl, —CN, —OH, —NH₂, —NH(C₁₋₃alkyl), —N(C₁₋₃alkyl)₂, —OC₁₋₃alkyl or cyclopropyl.

[63]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [62], wherein, R₄ₐ is independently selected from —F, —Cl, methyl, ethyl, propyl, isopropyl, —CH═CH₂, —C≡CH, —C≡CCH₃, —C≡CD, —CH₂C≡CH, —CHF₂, —CHF₂, —CF₃, —CH₂CF₃, —CH₂CHF₂, —CH₂CH₂F, —CH₂CH₂CH₂F, —OCF₃, —CN, —CH₂CN, —CH₂CH₂CN, —NH₂, —N(CH₃)₂, —NHCH₂CH₃, —CH₂—N(CH₃)₂, —C(═O)H, —C(═O)(CH₃), —OH, —CH₂OH, —CH₂CH₂OH, —CH₂CH₂CH₂OH, —OCH₃, —OC(CH₃)₂, —CH₂CH(CH₃)₂, —CH(CH₃)CH₂CH₃, —CH₂OCH₃, —CH₂CH₂OCH₃, —SH, —SCH₃, —SCF₃, —OCHF₂, —CH(CF)OCH₃, —C(CH₃)₂OH, —CF(CH₃)₂, —OCH(CH₃)₂, —C(═O)OH, —NO₂, cyclopropyl,

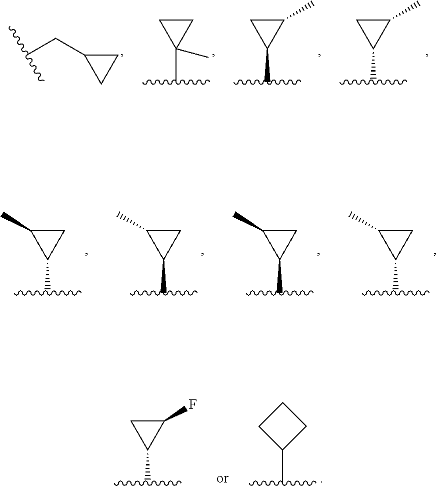

[64]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [63], wherein, R₄ is selected from any one in the Table 9.

[65]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [64], wherein, R₄ is selected from any one in the Table 17:

TABLE 17

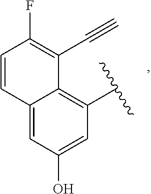 [SMILES: *c1cc(O)cc2ccc(F)c(C#C)c12]

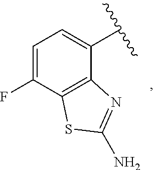 [SMILES: *c1ccc(F)c2sc(N)nc12]

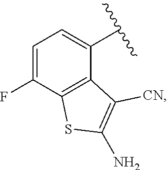 [SMILES: *c1ccc(F)c2sc(N)c(C#N)c12]

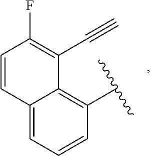 [SMILES: *c1cccc2ccc(F)c(C#C)c12]

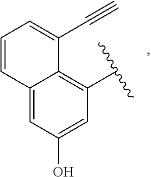 [SMILES: *c1cc(O)cc2cccc(C#C)c12]

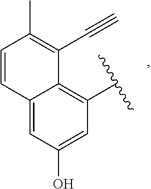 [SMILES: *c1cc(O)cc2ccc(C)c(C#C)c12]

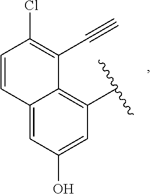 [SMILES: *c1cc(O)cc2ccc(Cl)c(C#C)c12]

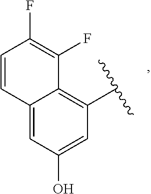 [SMILES: *c1cc(O)cc2ccc(F)c(F)c12]

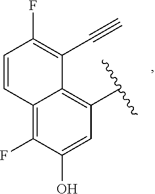 [SMILES: *c1cc(O)c(F)c2ccc(F)c(C#C)c12]

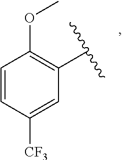 [SMILES: *c1cc(C(F)(F)F)ccc1OC]

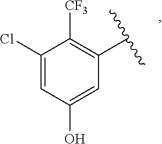 [SMILES: *c1cc(O)cc(Cl)c1C(F)(F)F]

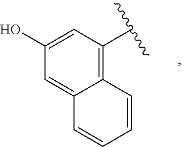 [SMILES: *c1cc(O)cc2ccccc12]

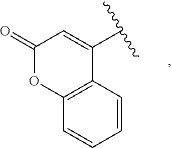 [SMILES: *c1cc(=O)oc2ccccc12]

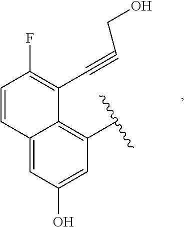 [SMILES: *c1cc(O)cc2ccc(F)c(C#CCO)c12]

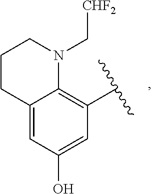 [SMILES: *c1cc(O)cc2c1N(CC(F)F)CCC2]

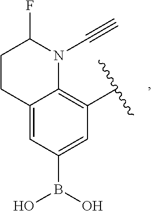 [SMILES: *c1cc(B(O)O)cc2c1N(C#C)C(F)CC2]

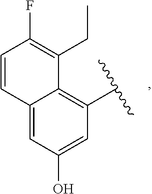 [SMILES: *c1cc(O)cc2ccc(F)c(CC)c12]

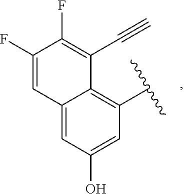 [SMILES: *c1cc(O)cc2cc(F)c(F)c(C#C)c12]

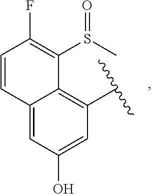 [SMILES: *c1cc(O)cc2ccc(F)c(S(C)=O)c12]

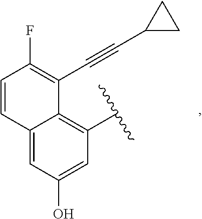 [SMILES: *c1cc(O)cc2ccc(F)c(C#CC3CC3)c12]

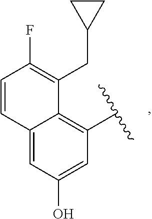 [SMILES: *c1cc(O)cc2ccc(F)c(CC3CC3)c12]

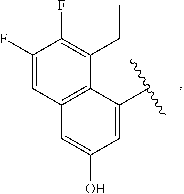 [SMILES: *c1cc(O)cc2cc(F)c(F)c(CC)c12]

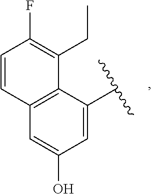 [SMILES: *c1cc(O)cc2ccc(F)c(CC)c12]

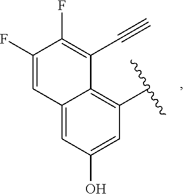 [SMILES: *c1cc(O)cc2cc(F)c(F)c(C#C)c12]

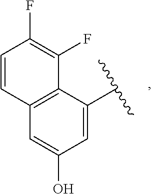 [SMILES: *c1cc(O)cc2ccc(F)c(F)c12]

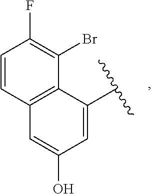 [SMILES: *c1cc(O)cc2ccc(F)c(Br)c12]

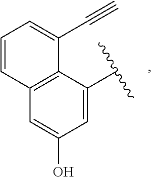 [SMILES: *c1cc(O)cc2cccc(C#C)c12]

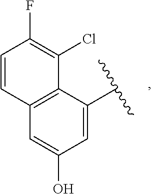 [SMILES: *c1cc(O)cc2ccc(F)c(Cl)c12]

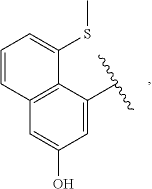 [SMILES: *c1cc(O)cc2cccc(SC)c12]

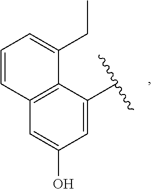 [SMILES: *c1cc(O)cc2cccc(CC)c12]

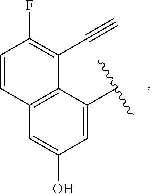 [SMILES: *c1cc(O)cc2ccc(F)c(C#C)c12]

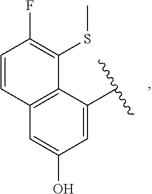 [SMILES: *c1cc(O)cc2ccc(F)c(SC)c12]

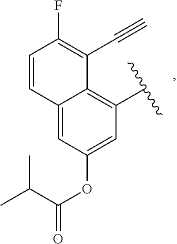 [SMILES: *c1cc(OC(=O)C(C)C)cc2ccc(F)c(C#C)c12]

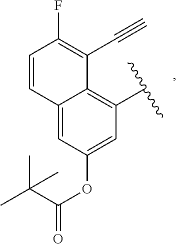 [SMILES: *c1cc(OC(=O)C(C)(C)C)cc2ccc(F)c(C#C)c12]

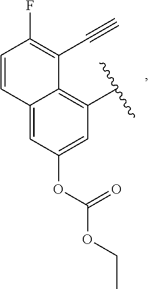 [SMILES: *c1cc(OC(=O)OCC)cc2ccc(F)c(C#C)c12]

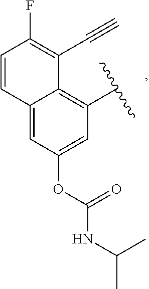 [SMILES: *c1cc(OC(=O)NC(C)C)cc2ccc(F)c(C#C)c12]

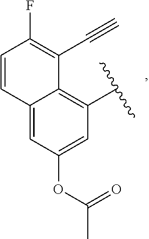 [SMILES: *c1cc(OC(C)=O)cc2ccc(F)c(C#C)c12]

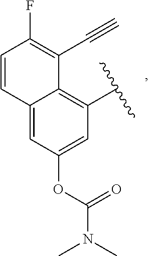 [SMILES: *c1cc(OC(=O)N(C)C)cc2ccc(F)c(C#C)c12]

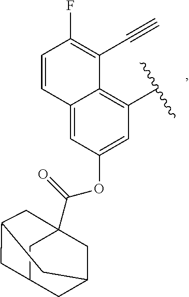 [SMILES: *c1cc(OC(=O)C23CC4CC(CC(C4)C2)C3)cc2ccc(F)c(C#C)c12]

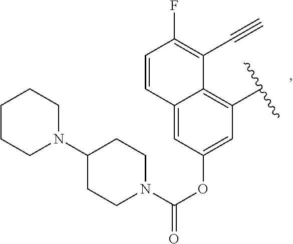 [SMILES: *c1cc(OC(=O)N2CCC(N3CCCCC3)CC2)cc2ccc(F)c(C#C)c12]

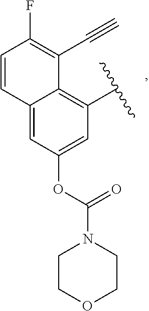 [SMILES: *c1cc(OC(=O)N2CCOCC2)cc2ccc(F)c(C#C)c12]

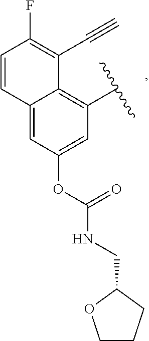 [SMILES: *c1cc(OC(=O)NC[C@@H]2CCCO2)cc2ccc(F)c(C#C)c12]

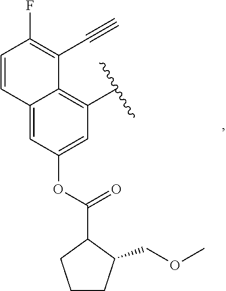 [SMILES: *c1cc(OC(=O)C2CCC[C@H]2COC)cc2ccc(F)c(C#C)c12]

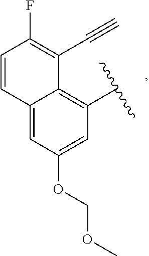 [SMILES: *c1cc(OCOC)cc2ccc(F)c(C#C)c12]

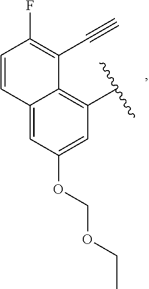 [SMILES: *c1cc(OCOCC)cc2ccc(F)c(C#C)c12]

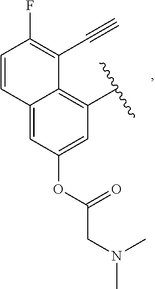 [SMILES: *c1cc(OC(=O)CN(C)C)cc2ccc(F)c(C#C)c12]

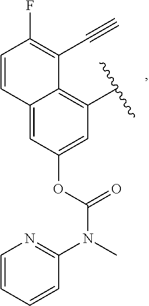 [SMILES: *c1cc(OC(=O)N(C)c2ccccn2)cc2ccc(F)c(C#C)c12]

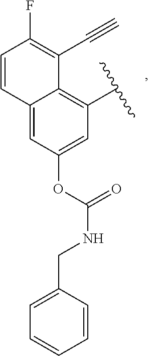 [SMILES: *c1cc(OC(=O)NCc2ccccc2)cc2ccc(F)c(C#C)c12]

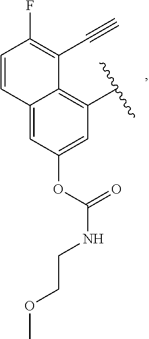 [SMILES: *c1cc(OC(=O)NCCOC)cc2ccc(F)c(C#C)c12]

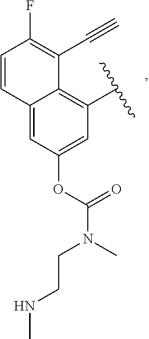 [SMILES: *c1cc(OC(=O)N(C)CCNC)cc2ccc(F)c(C#C)c12]

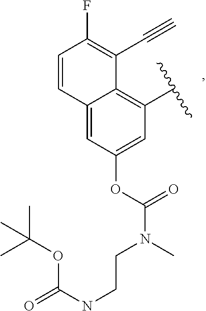 [SMILES: *c1cc(OC(=O)N(C)CCNC(=O)OC(C)(C)C)cc2ccc(F)c(C#C)c12]

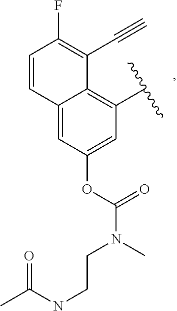 [SMILES: *c1cc(OC(=O)N(C)CCNC(C)=O)cc2ccc(F)c(C#C)c12]

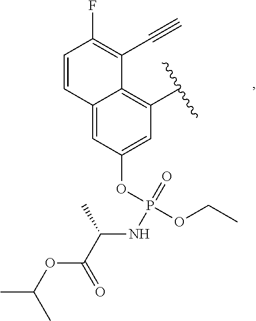 [SMILES: *c1cc(OP(=O)(N[C@@H](C)C(=O)OC(C)C)OCC)cc2ccc(F)c(C#C)c12]

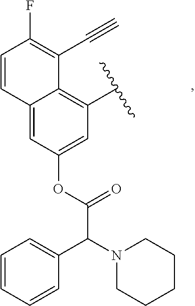 [SMILES: *c1cc(OC(=O)C(c2ccccc2)N2CCCCC2)cc2ccc(F)c(C#C)c12]

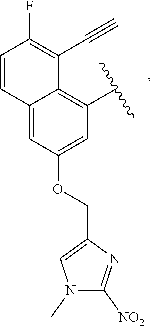 [SMILES: *c1cc(OCc2cn(C)c([N+](=O)[O-])n2)cc2ccc(F)c(C#C)c12]

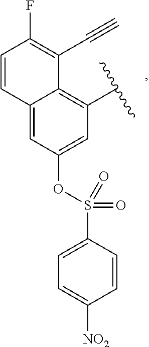 [SMILES: *c1cc(OS(=O)(=O)c2ccc([N+](=O)[O-])cc2)cc2ccc(F)c(C#C)c12]

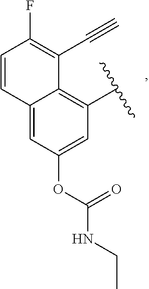 [SMILES: *c1cc(OC(=O)NCC)cc2ccc(F)c(C#C)c12]

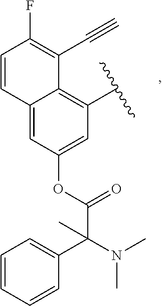 [SMILES: *c1cc(OC(=O)C(C)(c2ccccc2)N(C)C)cc2ccc(F)c(C#C)c12]

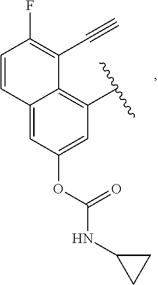 [SMILES: *c1cc(OC(=O)NC2CC2)cc2ccc(F)c(C#C)c12]

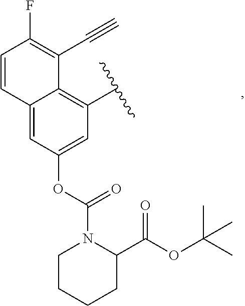 [SMILES: *c1cc(OC(=O)N2CCCCC2C(=O)OC(C)(C)C)cc2ccc(F)c(C#C)c12]

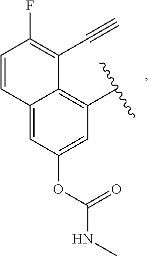 [SMILES: *c1cc(OC(=O)NC)cc2ccc(F)c(C#C)c12]

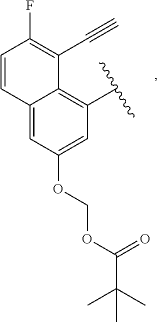 [SMILES: *c1cc(OCOC(=O)C(C)(C)C)cc2ccc(F)c(C#C)c12]

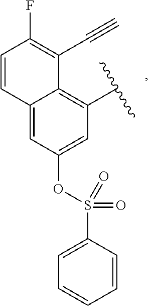 [SMILES: *c1cc(OS(=O)(=O)c2ccccc2)cc2ccc(F)c(C#C)c12]

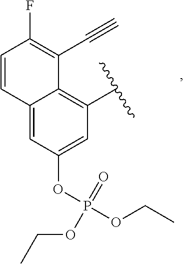 [SMILES: *c1cc(OP(=O)(OCC)OCC)cc2ccc(F)c(C#C)c12]

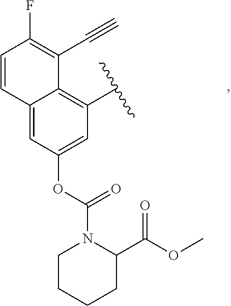 [SMILES: *c1cc(OC(=O)N2CCCCC2C(=O)OC)cc2ccc(F)c(C#C)c12]

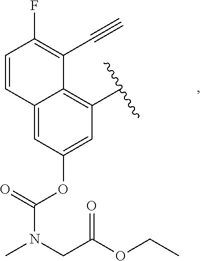 [SMILES: *c1cc(OC(=O)N(C)CC(=O)OCC)cc2ccc(F)c(C#C)c12]

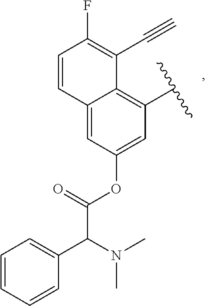 [SMILES: *c1cc(OC(=O)C(c2ccccc2)N(C)C)cc2ccc(F)c(C#C)c12]

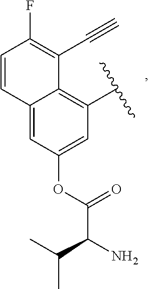 [SMILES: *c1cc(OC(=O)[C@@H](N)C(C)C)cc2ccc(F)c(C#C)c12]

or

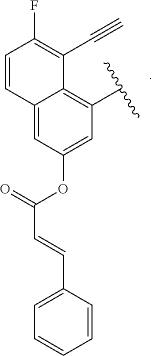 [SMILES: *c1cc(OC(=O)/C=C/c2ccccc2)cc2ccc(F)c(C#C)c12]

[66]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [65], wherein, R₅ is selected from hydrogen, halogen, —C₁₋₆alkyl, haloC₁₋₆alkyl, haloC₁₋₆alkoxy, —CN, —NR₅₁R₅₂, —OR₅₃, —SR₅₄, —S(haloC₁₋₆alkyl), 3-6 membered cycloalkyl or 3-6 membered heterocyclyl; wherein, said —C₁₋₆alkyl, 3-6 membered cycloalkyl or 3-6 membered heterocyclyl is optionally substituted with 1, 2 or 3 substituents selected from halogen, —C₁₋₆alkyl, haloC₁₋₆alkyl, haloC₁₋₆alkoxy, —C₂₋₆alkenyl, —C₂₋₆alkynyl, —CN, —NR₅₁R₅₂, —OR₅₃, —SR₅₄, —S(haloC₁₋₆alkyl), —S(═O)R₆₅, —S(═O)₂R₅₅, —C(═O)R₅₅, —C(═O)(OR₅₃), —OC(═O)(R₅₅), —C(═O)NR₅₁R₅₂, —NR₆₁C(═O)(R₅₅), —S(═O)₂NR₅₁R₅₂, —NR₅₁S(═O)₂R₅₅, 3-10 membered cycloalkyl, 3-10 membered heterocyclyl, 6-10 membered aryl or 5-10 membered heteroaryl;
R₅₁, R₅₂, R₅₃ and R₅₄ are each independently selected from hydrogen, —C₁₋₆alkyl or 3-6 membered cycloalkyl.

[67]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [36] to [66], wherein, R₅ is selected from hydrogen, halogen, —C₁₋₆alkyl, haloC₁₋₆alkyl, haloC₁₋₆alkoxy, —CN, —NHC₁₋₆alkyl, —N(C₁₋₆alkyl)₂, —OC₁₋₆alkyl, —O-(3-6 membered cycloalkyl), —SC₁₋₆alkyl, —S(haloC₁₋₆alkyl) or 3-6 membered cycloalkyl; wherein, said —C₁₋₆alkyl or 3-6 membered cycloalkyl is optionally substituted with 1, 2 or 3 substituents selected from halogen, —C₁₋₆alkyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl, —SH, —SC₁₋₆alkyl or —S(haloC₁₋₆alkyl).

[68]. The compound of formula (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of 36 to 67 wherein, 5 is selected from hydrogen,

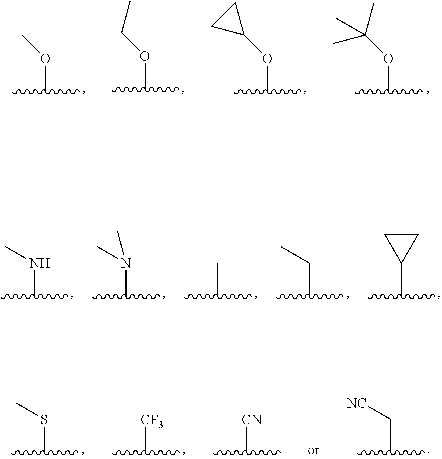

[69]. The compound of formula (I) or (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [1] to [68], wherein, the conjugated form is a PROTAC molecule.

[70]. The compound of formula (I) or (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [1] to [69] selected from any compound in the Table 18:

TABLE 18

| ID | IUPAC Name |
|---|---|
| 1 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-ol; |
| 2 | (R)-5-ethynyl-6-fluoro-4-(8-fluoro-2-((1-methylpyrrolidin- |
| | 2-yl)methoxy)-4-(1,4-oxazepan-4-yl)pyrido[4,3-d]pyrimidin- |
| | 7-yl)naphthalen-2-ol; |
| 3 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-yl acetate; |
| 4 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-yl dimethylcarbamate; |
| 5 | ethyl |
| | (5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-yl) carbonate; |
| 6 | 5-ethynyl-6-fluoro-4-(8-fluoro-2-((2R,7aS)-2-fluorotetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-yl palmitate; |
| 7 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido |
| | [4,3-d]pyrimidin-7-yl)-5-ethynyl-6-fluoronaphthalen-2-ol; |
| 8 | 4-(4-(1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl) |
| | methoxy)pyrido[4,3-d]pyrimidin-7-yl)-5-ethynyl-6- |
| | fluoronaphthalen-2-ol; |
| 9 | 4-(4-((1R,7S)-2-oxa-6-azabicyclo[5.1.0]octan-6-yl)-8-fluoro-2- |
| | ((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 10 | 5-ethynyl-6-fluoro-4-(8-fluoro-2-(((2R,7aS)-2-fluorotetrahydro- |
| | 1H-pyrrolizin-7a(5H)-yl)methoxy)-4-((S)-2-methyl-1,4- |
| | oxazepan-4-yl)pyrido[4,3-d]pyrimidin-7-yl)naphthalen-2-ol; |
| 11 | 5-ethynyl-6-fluoro-4-(8-fluoro-2-(((2R,7aS)-2-fluorotetrahydro- |
| | 1H-pyrrolizin-7a(5H)-yl)methoxy)-4-((R)-2-methyl-1,4- |
| | oxazepan-4-yl)pyrido[4,3-d]pyrimidin-7-yl)naphthalen-2-ol; |
| 12 | (S)-4-(2-((2,2-difluorotetrahydro-1H-pyrrolizin-7a(5H)-yl) |
| | methoxy)-8-fluoro-4-(1,4-oxazepan-4-yl)pyrido[4,3-d] |
| | pyrimidin-7-yl)-5-ethynyl-6-fluoronaphthalen-2-ol; |
| 13 | 5-ethynyl-6-fluoro-4-(8-fluoro-2-((2-fluoro-2-methyltetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-ol; |
| 14 | 5-ethynyl-6-fluoro-4-(8-fluoro-2-(((2R,7aS)-2-methoxytetrahydro- |
| | 1H-pyrrolizin-7a(5H)-yl)methoxy)-4-(1,4-oxazepan-4-yl) |
| | pyrido[4,3-d]pyrimidin-7-yl)naphthalen-2-ol; |
| 15 | 5-ethynyl-6-fluoro-4-(8-fluoro-4-(6-fluoro-1,4-oxazepan-4-yl)- |
| | 2-(((2R,7aS)-2-fluorotetrahydro-1H-pyrrolizin-7a(5H)-yl) |
| | methoxy)pyrido[4,3-d]pyrimidin-7-yl)naphthalen-2-ol; |
| 16 | 5-ethynyl-6-fluoro-4-(8-fluoro-4-((R)-6-fluoro-1,4-oxazepan- |
| | 4-yl)-2-(((2R,7aS)-2-fluorotetrahydro-1H-pyrrolizin-7a(5H)-yl) |
| | methoxy)pyrido[4,3-d]pyrimidin-7-yl)naphthalen-2-ol; |
| 17 | 5-ethynyl-6-fluoro-4-(8-fluoro-4-((S)-6-fluoro-1,4-oxazepan- |
| | 4-yl)-2-(((2R,7aS)-2-fluorotetrahydro-1H-pyrrolizin-7a(5H)-yl) |
| | methoxy)pyrido[4,3-d]pyrimidin-7-yl)naphthalen-2-ol; |
| 18 | 5-ethynyl-6-fluoro-4-(8-fluoro-2-(((2R,7aS)-2-fluorotetrahydro- |
| | 1H-pyrrolizin-7a(5H)-yl)methoxy)-4-(6-methyl-1,4-oxazepan- |
| | 4-yl)pyrido[4,3-d]pyrimidin-7-yl)naphthalen-2-ol; |
| 19 | 5-ethynyl-6-fluoro-4-(8-fluoro-2-(((2R,7aS)-2-fluorotetrahydro- |
| | 1H-pyrrolizin-7a(5H)-yl)methoxy)-4-((R)-6-methyl-1,4-oxazepan- |
| | 4-yl)pyrido[4,3-d]pyrimidin-7-yl)naphthalen-2-ol; |
| 20 | 5-ethynyl-6-fluoro-4-(8-fluoro-2-(((2R,7aS)-2-fluorotetrahydro- |
| | 1H-pyrrolizin-7a(5H)-yl)methoxy)-4-((S)-6-methyl-1,4-oxazepan- |
| | 4-yl)pyrido[4,3-d]pyrimidin-7-yl)naphthalen-2-ol; |
| 21 | 2-(4-(7-(8-ethynyl-7-fluoro-3-hydroxynaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepan-6-yl) |
| | acetonitrile; |
| 22 | 2-((R)-4-(7-(8-ethynyl-7-fluoro-3-hydroxynapththalen-1-yl)-8- |
| | fluoro-2-(((2R,7aS)-2-fluorotetrahydro-1H-pyrrolizin-7a(5H)- |
| | yl)methoxy)pyrido[4,3-d]pyrimidin-4-yl)-1,4-oxazepan-6-yl) |
| | acetonitrile; |
| 23 | 2-((S)-4-(7-(8-ethynyl-7-fluoro-3-hydroxynaphthalen-1-yl)-8- |
| | fluoro-2-(((2R,7aS)-2-fluorotetrahydro-1H-pyrrolizin-7a(5H)- |
| | yl)methoxy)pyrido[4,3-d]pyrimidin-4-yl)-1,4-oxazepan-6-yl) |
| | acetonitrile; |
| 24 | 2-(4-(7-(8-ethynyl-7-fluoro-3-hydroxynaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepan-2-yl) |
| | acetonitrile; |
| 25 | 2-((S)-4-(7-(8-ethynyl-7-fluoro-3-hydroxynaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepan-2-yl) |
| | acetonitrile; |
| 26 | 2-((R)-4-(7-(8-ethynyl-7-fluoro-3-hydroxynaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepan-2-yl) |
| | acetonitrile; |
| 27 | 5-ethynyl-6-fluoro-4-(8-fluoro-2-(((2R,7aS)-2-fluorotetrahydro- |
| | 1H-pyrrolizin-7a(5H)-yl)methoxy)-4-(3-methyl-1,4-oxazepan- |
| | 4-yl)pyrido[4,3-d]pyrimidin-7-yl)naphthalen-2-ol; |
| 28 | 5-ethynyl-6-fluoro-4-(8-fluoro-2-(((2R,7aS)-2-fluorotetrahydro- |
| | 1H-pyrrolizin-7a(5H)-yl)methoxy)-4-((R)-3-methyl-1,4- |
| | oxazepan-4-yl)pyrido[4,3-d]pyrimidin-7-yl)naphthalen-2-ol; |
| 29 | 5-ethynyl-6-fluoro-4-(8-fluoro-2-(((2R,7aS)-2-fluorotetrahydro- |
| | 1H-pyrrolizin-7a(5H)-yl)methoxy)-4-((S)-3-methyl-1,4-oxazepan- |
| | 4-yl)pyrido[4,3-d]pyrimidin-7-yl)naphthalen-2-ol; |
| 30 | 5-ethynyl-6-fluoro-4-(8-fluoro-2-(((2R,7aS)-2-fluorotetrahydro- |
| | 1H-pyrrolizin-7a(5H)-yl)methoxy)-4-(7-methyl-1,4-oxazepan- |
| | 4-yl)pyrido[4,3-d]pyrimidin-7-yl)naphthalen-2-ol; |
| 31 | 4-(4-(6,6-difluoro-1,4-oxazepan-4-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 32 | 4-(6-chloro-8-fluoro-2-(((2R,7aS)-2-fluorotetrahydro-1H- |
| | pyrrolizin-7a(5H)-yl)methoxy)-4-(1,4-oxazepan-4-yl) |
| | quinazolin-7-yl)-5-ethynyl-6-fluoronaphthalen-2-ol; |
| 33 | (P)-4-(6-chloro-8-fluoro-2-(((2R,7aS)-2-fluorotetrahydro-1H- |
| | pyrrolizin-7a(5H)-yl)methoxy)-4-(1,4-oxazepan-4-yl)quinazolin- |
| | 7-yl)-5-ethynyl-6-fluoronaphthalen-2-ol; |
| 34 | (M)-4-(6-chloro-8-fluoro-2-(((2R,7aS)-2-fluorotetrahydro-1H- |
| | pyrrolizin-7a(5H)-yl)methoxy)-4-(1,4-oxazepan-4-yl)quinazolin- |
| | 7-yl)-5-ethynyl-6-fluoronaphthalen-2-ol; |
| 35 | 5-ethynyl-6-fluoro-4-(8-fluoro-2-((1-(morpholinomethyl)cyclo- |
| | propyl)methoxy)-4-(1,4-oxazepan-4-yl)quinazolin-7-yl) |
| | naphthalen-2-ol; |
| 36 | 5-ethynyl-6-fluoro-4-(8-fluoro-4-(1,4-oxazepan-4-yl)-2- |
| | ((tetrahydro-1H-pyrrolizin-7a(5H)-yl)methoxy)pyrido[4,3-d] |
| | pyrimidin-7-yl)naphthalen-2-ol; |
| 37 | 5-ethynyl-6-fluoro-4-(8-fluoro-2-(((2R,7aS)-2-fluorotetrahydro- |
| | 1H-pyrrolizin-7a(5H)-yl)methoxy)-4-(5-methyl-1,4-oxazepan- |
| | 4-yl)pyrido[4,3-d]pyrimidin-7-yl)naphthalen-2-ol; |
| 38 | 4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-6-methyl-1,4-oxazepan-6-ol; |
| 39 | 5-ethynyl-6-fluoro-4-(8-fluoro-2-(((2R,7aS)-2-fluorotetrahydro- |
| | 1H-pyrrolizin-7a(5H)-yl)methoxy)-4-(6-methylene-1,4- |
| | oxazepan-4-yl)pyrido[4,3-d]pyrimidin-7-yl)naphthalen-2-ol; |
| 40 | (E)-4-(7-(8-ethynyl-7-fluoro-3-hydroxynaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepan-6-one |
| | O-methyl oxime; |
| 41 | 4-(4-(6-dimethylamino)-1,4-oxazepan-4-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 42 | 4-(4-(3,6-dimethyl-1,4-oxazepan-4-yl)-8-fluoro-2-(((2R,7aS)- |
| | 2-fluorotetrahydro-1H-pyrrolizin-7a(5H)-yl)methoxy)pyrido |
| | [4,3-d]pyrimidin-7-yl)-5-ethynyl-6-fluoronaphthalen-2-ol; |
| 43 | 4-(4-(2-oxa-6-azabicyclo[5.1.0]octan-6-yl)-8-fluoro-2-((1- |
| | (morpholinomethyl)cyclopropyl)methoxy)pyrido[4,3-d] |
| | pyrimidin-7-yl)-5-ethynyl-6-fluoronaphthalen-2-ol; |
| 44 | 4-(4-((1S,7R)-2-oxa-6-azabicyclo[5.1.0]octan-6-yl)-8-fluoro- |
| | 2-((1-morpholinomethyl)cyclopropyl)methoxy)pyrido[4,3-d] |
| | pyrimidin-7-yl)-5-ethynyl-6-fluoronaphthalen-2-ol; |
| 45 | 4-(4-((1R,7S)-2-oxa-6-azabicyclo[5.1.0]octan-6-yl)-8-fluoro-2- |
| | ((1-(morpholinomethyl)cyclopropyl)methoxy)pyrido[4,3-d] |
| | pyrimidin-7-yl)-5-ethynyl-6-fluoronaphthalen-2-ol; |
| 46 | 5-ethynyl-6,7-difluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 47 | 4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepan-6-one; |
| 48 | 5-ethynyl-6-fluoro-4-(8-fluoro-4-(6-fluoro-3-methyl-1,4-oxazepan- |
| | 4-yl)-2-(((2R,7aS)-2-fluorotetrahydro-1H-pyrrolizin-7a(5H)-yl) |
| | methoxy)pyrido[4,3-d]pyrimidin-7-yl)naphthalen-2-ol; |
| 49 | 5-ethynyl-6-fluoro-4-(8-fluoro-2-(((2R)-2-fluoro-2-methyltetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 50 | 5-ethynyl-6-fluoro-4-(8-fluoro-2-(((2S)-2-fluoro-2-methyltetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 51 | 4-(4-(5-oxa-2-azabicyclo[5.1.0]octan-2-yl)-8-fluoro-2-(((2R,7aS)- |
| | 2-fluorotetrahydro-1H-pyrrolizin-7a(5H)-yl)methoxy)pyrido |
| | [4,3-d]pyrimidin-7-yl)-5-ethynyl-6-fluoronaphthalen-2-ol; |
| 52 | 4-(5-(difluoromethyl)-8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)-5-ethynyl-6-fluoronaphthalen-2-ol; |
| 53 | 4-(5-cyclopropyl-8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)-5-ethynyl-6-fluoronaphthalen-2-ol; |
| 54 | 5-ethynyl-6-fluoro-4-(8-fluoro-2-(((2R,7aS)-2-fluorotetrahydro-1H- |
| | pyrrolizin-7a(5H)-yl)methoxy)-5-(hydroxymethyl)-4-(1,4-oxazepan- |
| | 4-yl)pyrido[4,3-d]pyrimidin-7-yl)naphthalen-2-ol; |
| 55 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)-6- |
| | (trifluoromethyl)quinazolin-7-yl)naphthalen-2-ol; |
| 56 | 5-ethynyl-4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)-6-methylnaphthalen-2-ol; |
| 57 | 6-chloro-5-ethynyl-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 58 | 5,6-difluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 59 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)-6- |
| | ((trifluoromethyl)thio)quinazolin-7-yl)naphthalen-2-ol; |
| 60 | 4-(4-(3-oxa-7-azabicyclo[4.1.1]octan-7-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 61 | 5-ethynyl-6-fluoro-4-(8-fluoro-2-(((2R,7aS)-2-fluorotetrahydro-1H- |
| | pyrrolizin-7a(5H)-yl)methoxy)-4-(7-oxa-4-azaspiro[2.6]nonan-4-yl) |
| | pyrido[4,3-d]pyrimidin-7-yl)naphthalen-2-ol; |
| 62 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl acetyl-L-tryptophanate; |
| 63 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl isobutyrate; |
| 64 | (R)-4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepan-6-ol; |
| 65 | (S)-4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepan-6-ol; |
| 66 | 5-ethynyl-6-fluoro-4-(8-fluoro-2-(((2R,7aS)-2-fluorotetrahydro-1H- |
| | pyrrolizin-7a(5H)-yl)methoxy)-5-(methylthio)-4-(1,4-oxazepan-4-yl) |
| | pyrido[4,3-d]pyrimidin-7-yl)naphthalen-2-ol; |
| 67 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl cinnamate; |
| 68 | 5-ethynyl-6-fluoro-4-(8-fluoro-2-(((2R,7aS)-2-fluorotetrahydro-1H- |
| | pyrrolizin-7a(5H)-yl)methoxy)-4-(6-methoxy-1,4-oxazepan-4-yl) |
| | pyrido[4,3-d]pyrimidin-7-yl)naphthalen-2-ol; |
| 69 | 5-ethynyl-6-fluoro-4-(8-fluoro-2-(((2R,7aS)-2-fluorotetrahydro-1H- |
| | pyrrolizin-7a(5H)-yl)methoxy)-4-((R)-6-methoxy-1,4-oxazepan-4-yl) |
| | pyrido[4,3-d]pyrimidin-7-yl)naphthalen-2-ol; |
| 70 | 5-ethynyl-6-fluoro-4-(8-fluoro-2-(((2R,7aS)-2-fluorotetrahydro-1H- |
| | pyrrolizin-7a(5H)-yl)methoxy)-4-((S)-6-methoxy-1,4-oxazepan-4-yl) |
| | pyrido[4,3-d]pyrimidin-7-yl)naphthalen-2-ol; |
| 71 | 5-ethynyl-6-fluoro-4-(8-fluoro-2-(((2R,7aS)-2-fluorotetrahydro-1H- |
| | pyrrolizin-7a(5H)-yl)methoxy)-4-(8-oxa-4-azaspiro[2.6]nonan-4-yl) |
| | pyrido[4,3-d]pyrimidin-7-yl)naphthalen-2-ol; |
| 72 | 4-(5-cyclopropoxy-8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)-5-ethynyl-6-fluoronaphthalen-2-ol; |
| 73 | 5-ethynyl-6-fluoro-4-(8-fluoro-2-(((2R,7aS)-2-fluorotetrahydro-1H- |
| | pyrrolizin-7a(5H)-yl)methoxy)-5-isopropoxy-4-(1,4-oxazepan-4-yl) |
| | pyrido[4,3-d]pyrimidin-7-yl)naphthalen-2-ol; |
| 74 | 5-ethynyl-6-fluoro-4-(8-fluoro-2-(((2R,7aS)-2-fluorotetrahydro-1H- |
| | pyrrolizin-7a(5H)-yl)methoxy)-5-(methylamino)-4-(1,4-oxazepan- |
| | 4-yl)pyrido[4,3-d]pyrimidin-7-yl)naphthalen-2-ol; |
| 75 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl |
| | (methyl(2-(methylamino)ethyl)carbamate; |
| 76 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl 2-phenyl-2-(piperidin-1-yl)acetate; |
| 77 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl 4-nitrobenzenesulfonate; |
| 78 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl ethylcarbamate; |
| 79 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl |
| | 2-(dimethylamino)-2-phenylpropanoate; |
| 80 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl 2-(dimethylamino)-2-phenylacetate; |
| 81 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl L-valinate; |
| 82 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl dimethylglycinate; |
| 83 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl |
| | (R)-4-((3R,5S,7R,8R,9S,10S,12S,13R,14S,17R)-3,7,12-trihydroxy- |
| | 10,13-dimethylhexadecahydro-1H-cyclopenta[a]phenanthren-17-yl) |
| | pentanoate; |
| 84 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl |
| | 5-((3aS,4S,6aR)-2-oxohexahydro-1H-thieno[3,4-d]imidazol-4-yl) |
| | pentanoate; |
| 85 | 5-ethynyl-6-fluoro-4-(8-fluoro-2-((1-(morpholinomethyl)cyclo- |
| | propyl)methoxy)-4-(1,4-oxazepan-4-yl)pyrido[4,3-d]pyrimidin-7-yl) |
| | naphthalen-2-ol; |
| 86 | 5-ethynyl-6-fluoro-4-(8-fluoro-2-(((2R,7aS)-2-fluorotetrahydro-1H- |
| | pyrrolizin-7a(5H)-yl)methoxy)-5-methoxy-4-(1,4-oxazepan-4-yl) |
| | pyrido[4,3-d]pyrimidin-7-yl)naphthalen-2-ol; |
| 87 | 4-(5-ethoxy-8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)-5-ethynyl-6-fluoronaphthalen-2-ol; |
| 88 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl methylcarbamate; |
| 89 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl benzenesulfonate; |
| 90 | 4-(7-(8-ethynyl-7-fluoro-3-(methoxymethoxy)naphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-3-methyl-1,4-oxazepane; |
| 91 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl 2-amino-6-methylbenzoate; |
| 92 | ethyl |
| | N-(((5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl)oxy)carbonyl)-N- |
| | methylglycinate; |
| 93 | 1-(5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl) 2-methyl-piperidine-1,2- |
| | dicarboxylate; |
| 94 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl pivalate; |
| 95 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl adamantane-1-carboxylate; |
| 96 | diethyl |
| | (5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl) phosphate; |
| 97 | ((5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl)oxy)methyl pivalate; |
| 98 | 4-(4-((1S,7R)-5-oxa-2-azabicyclo[5.1.0]octan-2-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 99 | 4-(4-((1R,7S)-5-oxa-2-azabicyclo[5.1.0]octan-2-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 100 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)-5- |
| | methoxypyrido[4,3-d]pyrimidin-7-yl)-5-ethynyl-6-fluoronaphthalen- |
| | 2-ol; |
| 101 | 4-(4-(2-oxa-6-azabicyclo[5.1.0]octan-6-yl)-5-ethoxy-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 102 | 2-amino-7-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)benzo[b]thiophene-3-carbonitrile; |
| 103 | 4-(7-(8-ethynyl-7-fluoronaphthalen-1-yl)-8-fluoro-2-(((2R,7aS)-2- |
| | fluorotetrahydro-1H-pyrrolizin-7a(5H)-yl)methoxy)pyrido[4,3-d] |
| | pyrimidin-4-yl)-1,4-oxazepane; |
| 104 | 5-ethynyl-4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)naphthalen-2-ol; |
| 105 | 4-(2-((2-(dimethylamino)tetrahydro-1H-pyrrolizin-7a(5H)-yl) |
| | methoxy)-8-fluoro-4-(1,4-oxazepan-4-yl)pyrido[4,3-d]pyrimidin- |
| | 7-yl)-5-ethynyl-6-fluoronaphthalen-2-ol; |
| 106 | 4-(4-(6-oxa-3-azabicyclo[3.2.1]octan-3-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 107 | 4-(4-((1R,5S)-6-oxa-3-azabicyclo[3.2.1]octan-3-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 108 | 4-(4-((1S,5R)-6-oxa-3-azabicyclo[3.2.1]octan-3-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 109 | 4-(4-(9-oxa-3-azabicyclo[4.2.1]nonan-3-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 110 | (1S,5S,8S)-3-(7-(8-ethynyl-7-fluoro-3-hydroxynaphthalen-1-yl)- |
| | 8-fluoro-2-(((2R,7aS)-2-fluorotetrahydro-1H-pyrrolizin-7a(5H)-yl) |
| | methoxy)pyrido[4,3-d]pyrimidin-4-yl)-6-oxa-3-azabicyclo[3.2.1] |
| | octan-8-ol; |
| 111 | 5-ethynyl-6-fluoro-4-(8-fluoro-2-(((2R,7aS)-2-fluorotetrahydro-1H- |
| | pyrrolizin-7a(5H)-yl)methoxy)-4-(6-(methoxymethyl)-1,4-oxazepan- |
| | 4-yl)pyrido[4,3-d]pyrimidin-7-yl)naphthalen-2-ol; |
| 112 | 4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepane-6-carbonitrile; |
| 113 | 4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl) |
| | methoxy)pyrido[4,3-d]pyrimidin-4-yl)-1,4-oxazepan-3-one; |
| 114 | (5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-7-yl)boronic acid; |
| 115 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl [1,4′-bipiperidine]-1′-carboxylate; |
| 116 | 4-(4-(6-oxa-2-azabicyclo[3.2.1]octan-2-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 117 | 4-(4-((1R,5S)-6-oxa-2-azabicyclo[3.2.1]octan-2-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 118 | 4-(4-((1S,5R)-6-oxa-2-azabicyclo[3.2.1]octan-2-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-7-yl)-5-ethynyl-6- |
| | fluoronaphthalen-2-ol; |
| 119 | 4-(4-(3-oxa-6-azabicyclo[3.2.1]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 120 | 4-(4-((1R,5R)-3-oxa-6-azabicyclo[3.2.1]octan-6-yl)-8-fluoro-2- |
| | ((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 121 | 4-(4-((1S,5S)-3-oxa-6-azabicyclo[3.2.1]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 122 | 4-(2-((1,4-diazabicyclo[2.2.2]octan-2-yl)methoxy)-8-fluoro-4- |
| | (1,4-oxazepan-4-yl)pyrido[4,3-d]pyrimidin-7-yl)-5-ethynyl-6- |
| | fluoronaphthalen-2-ol; |
| 123 | 5-ethynyl-6-fluoro-4-(8-fluoro-2-((1-methylpiperazin-2-yl) |
| | methoxy)-4-(1,4-oxazepan-4-yl)pyrido[4,3-d]pyrimidin-7-yl) |
| | naphthalen-2-ol; |
| 124 | 5-ethynyl-6-fluoro-4-(8-fluoro-4-(1,4-oxazepan-4-yl)-2-(2- |
| | (tetrahydro-1H-pyrrolizin-7a(5H)-yl)ethoxy)pyrido[4,3-d] |
| | pyrimidin-7-yl)naphthalen-2-ol; |
| 125 | 1-(3-(((7-(8-ethynyl-7-fluoro-3-hydroxynaphthalen-1-yl)-8-fluoro- |
| | 4-(1,4-oxazepan-4-yl)pyrido[4,3-d]pyrimidin-2-yl)oxy)methyl)- |
| | 4-methylpiperazin-1-yl)prop-2-en-1-one; |
| 126 | 5-ethynyl-6-fluoro-4-(8-fluoro-4-(1,4-oxazepan-4-yl)-2-(quinuclidin- |
| | 2-ylmethoxy)pyrido[4,3-d]pyrimidin-7-yl)naphthalen-2-ol; |
| 127 | 5-ethynyl-6-fluoro-4-(8-fluoro-2-((2-(methylthio)tetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 128 | 5-ethynyl-6-fluoro-4-(8-fluoro-4-(6-fluoro-6-methyl-1,4-oxazepan- |
| | 4-yl)-2-(((2R,7aS)-2-fluorotetrahydro-1H-pyrrolizin-7a(5H)-yl) |
| | methoxy)pyrido[4,3-d]pyrimidin-7-yl)naphthalen-2-ol; |
| 129 | 4-(4-(6,6-dimethyl-1,4-oxazepan-4-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 130 | 5-ethynyl-6-fluoro-4-(8-fluoro-2-(((2R,7aS)-2-fluorotetrahydro-1H- |
| | pyrrolizin-7a(5H)-yl)methoxy)-4-(5-oxa-8-azaspiro[2.6]nonan-8-yl) |
| | pyrido[4,3-d]pyrimidin-7-yl)naphthalen-2-ol; |
| 131 | 5-ethynyl-6-fluoro-4-(8-fluoro-2-(((2R,7aS)-2-fluorortetrahydro- |
| | 1H-pyrrolizin-7a(5H)-yl)methoxy)-4-(6-oxa-9-azaspiro[3.6]decan- |
| | 9-yl)pyrido[4,3-d]pyrimidin-7-yl)naphthalen-2-ol; |
| 132 | 4-(4-(6,6-difluoro-3-methyl-1,4-oxazepan-4-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 133 | 5-ethynyl-6-fluoro-4-(8-fluoro-2-(((2R,7aS)-2-fluorotetrahydro-1H- |
| | pyrrolizin-7a(5H)-yl)methoxy)-4-(6-methoxy-3-methyl-1,4- |
| | oxazepan-4-yl)pyrido[4,3-d]pyrimidin-7-yl)naphthalen-2-ol; |
| 134 | 5-ethynyl-1,6-difluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 135 | 4-(7-(8-ethynyl-7-fluoronaphthalen-1-yl)-8-fluoro-2-(((2R,7aS)-2- |
| | fluorotetrahydro-1H-pyrrolizin-7a(5H)-yl)methoxy)-5- |
| | methoxypyrido[4,3-d]pyrimidin-4-yl)-1,4-oxazepane; |
| 136 | 5-ethynyl-6-fluoro-4-(8-fluoro-2-(((2R,7aS)-2-fluorotetrahydro-1H- |
| | pyrrolizin-7a(5H)-yl)methoxy)-4-(6-methoxy-6-methyl-1,4- |
| | oxazepan-4-yl)pyrido[4,3-d]pyrimidin-7-yl)naphthalen-2-ol; |
| 137 | 5-ethynyl-6-fluoro-4-(8-fluoro-2-(((2R,7aS)-2-fluorotetrahydro-1H- |
| | pyrrolizin-7a(5H)-yl)methoxy)-4-((S)-6-methoxy-6-methyl-1,4- |
| | oxazepan-4-yl)pyrido[4,3-d]pyrimidin-7-yl)naphthalen-2-ol; |
| 138 | 5-ethynyl-6-fluoro-4-(8-fluoro-2-(((2R,7aS)-2-fluorotetrahydro-1H- |
| | pyrrolizin-7a(5H)-yl)methoxy)-4-((R)-6-methoxy-6-methyl-1,4- |
| | oxazepan-4-yl)pyrido[4,3-d]pyrimidin-7-yl)naphthalen-2-ol; |
| 139 | 5-ethynyl-6-fluoro-4-(8-fluoro-2-(((2R,7aS)-2-fluorotetrahydro-1H- |
| | pyrrolizin-7a(5H)-yl)methoxy)-4-((1R,5R,6S)-5-methyl-3-oxa-7- |
| | azabicyclo[4.1.1]octan-7-yl)pyrido[4,3-d]pyrimidin-7-yl) |
| | naphthalen-2-ol; |
| 140 | 5-ethynyl-6-fluoro-4-(8-fluoro-2-(((2R,7aS)-2-fluorotetrahydro-1H- |
| | pyrrolizin-7a(5H)-yl)methoxy)-4-((1R,5S,6S)-5-methyl-3-oxa-7- |
| | azabicyclo[4.1.1]octan-7-yl)pyrido[4,3-d]pyrimidin-7-yl) |
| | naphthalen-2-ol; |
| 141 | 4-(4-(2-oxa-6-azabicyclo[5.1.0]octan-6-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-7-fluorobenzo[d]thiazol-2-amine; |
| 142 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-7-fluorobenzo[d]thiazol-2-amine; |
| 143 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-7-fluorobenzo[d]thiazol-2-amine; |
| 144 | 4-(4-(2-oxa-6-azabicyclo[5.1.0]octan-6-yl)-8-fluoro-2-(((S)-1- |
| | methylpyrrolidin-2-yl)methoxy)pyrido[4,3-d]pyrimidin-7-yl)- |
| | 5-ethynyl-6-fluoronaphthalen-2-ol; |
| 145 | 4-(4-((1S,7R)-2-oxa-6-azabicyclo[5.1.0]octan-6-yl)-8-fluoro-2- |
| | (((S)-1-methylpyrrolidin-2-yl)methoxy)pyrido[4,3-d]pyrimidin-7-yl)- |
| | 5-ethynyl-6-fluoronaphthalen-2-ol; |
| 146 | 4-(4-((1R,7S)-2-oxa-6-azabicyclo[5.1.0]octan-6-yl)-8-fluoro-2- |
| | (((S)-1-methylpyrrolidin-2-yl)methoxy)pyrido[4,3-d]pyrimidin- |
| | 7-yl)-5-ethynyl-6-fluoronaphthalen-2-ol; |
| 147 | 4-(4-(2-oxa-6-azabicyclo[5.1.0]octan-6-yl)-8-fluoro-2-((1- |
| | (morpholinomethyl)cyclopropyl)methoxy)pyrido[4,3-d]pyrimidin- |
| | 7-yl)-7-fluorobenzo[d]thiazol-2-amine; |
| 148 | 6-(7-(8-ethynyl-7-fluoronaphthalen-1-yl)-8-fluoro-2-(((2R,7aS)- |
| | 2-fluorotetrahydro-1H-pyrrolizin-7a(5H)-yl)methoxy)pyrido[4,3-d] |
| | pyrimidin-4-yl)-2-oxa-6-azabicyclo[5.1.0]octane; |
| 149 | 4-(4-(2-oxa-6-azabicyclo[5.1.0]octan-6-yl)-6-chloro-8-fluoro-2- |
| | (((2R,7aS)-2-fluorotetrahydro-1H-pyrrolizin-7a(5H)-yl)methoxy) |
| | quinazolin-7-yl)-7-fluorobenzo[d]thiazol-2-amine; |
| 150 | 4-(4-(2-oxa-6-azabicyclo[5.1.0]octan-6-yl)-6-chloro-8-fluoro-2- |
| | ((1-(morpholinomethyl)cyclopropyl)methoxy)quinazolin-7-yl)-7- |
| | fluorobenzo[d]thiazol-2-amine; |
| 151 | 4-(8-fluoro-2-(((2R,7aS)-2-fluorotetrahydro-1H-pyrrolizin-7a(5H)- |
| | yl)methoxy)-4-(1,4-oxazepan-4-yl)pyrido[4,3-d]pyrimidin-7-yl) |
| | naphthalen-2-ol; |
| 152 | 4-(8-fluoro-2-(((2R,7aS)-2-fluorotetrahydro-1H-pyrrolizin-7a(5H)- |
| | yl)methoxy)-4-(1,4-oxazepan-4-yl)pyrido[4,3-d]pyrimidin-7-yl)- |
| | 2H-chromen-2-one; |
| 153 | 6-fluoro-4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)-5-(3-hydroxyprop-1-yn-1-yl)naphthalen-2-ol; |
| 154 | 5-ethynyl-6-fluoro-4-(8-fluoro-2-(((2R,7aS)-2-fluorotetrahydro-1H- |
| | pyrrolizin-7a(5H)-yl)methoxy)-5-methyl-4-(1,4-oxazepan-4-yl) |
| | pyrido[4,3-d]pyrimidin-7-yl)naphthalen-2-ol; |
| 155 | 4-(5-(dimethylamino)-8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)-5-ethynyl-6-fluoronaphthalen-2-ol; |
| 156 | 7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy)- |
| | 4-(1,4-oxazepan-4-yl)pyrido[4,3-d]pyrimidin-5-ol; |
| 157 | 4-(4-((E)-6-ethylidene-1,4-oxazepan-4-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 158 | 5-ethynyl-6-fluoro-4-(8-fluoro-2-(((2R,7aS)-2-fluorotetrahydro-1H- |
| | pyrrolizin-7a(5H)-yl)methoxy)-4-(6-(trifluoromethyl)-1,4-oxazepan- |
| | 4-yl)pyrido[4,3-d]pyrimidin-7-yl)naphthalen-2-ol; |
| 159 | 1-(2,2-difluoroethyl)-8-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)-1,2,3,4-tetrahydroquinolin-6-ol; |
| 160 | 5-ethynyl-6-fluoro-4-(8-fluoro-2-(((2R,7aS)-2-fluorotetrahydro-1H- |
| | pyrrolizin-7a(5H)-yl)methoxy)-5-(methylsulfinyl)-4-(1,4-oxazepan- |
| | 4-yl)pyrido[4,3-d]pyrimidin-7-yl)naphthalen-2-ol; |
| 161 | 5-ethynyl-6-fluoro-4-(8-fluoro-2-(((2R,7aS)-2-fluorotetrahydro-1H- |
| | pyrrolizin-7a(5H)-yl)methoxy)-5-(methylsulfonyl)-4-(1,4-oxazepan- |
| | 4-yl)pyrido[4,3-d]pyrimidin-7-yl)naphthalen-2-ol; |
| 162 | 7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy)- |
| | 4-(1,4-oxazepan-4-yl)pyrido[4,3-d]pyrimidine-5-carbonitrile; |
| 163 | 7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy)- |
| | 4-(1,4-oxazepan-4-yl)pyrido[4,3-d]pyrimidine-5-carboxylic acid; |
| 164 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl methyl(pyridin-2-yl)carbamate; |
| 165 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl benzylcarbamate; |
| 166 | 2-(tert-butyl) |
| | 1-(5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl) |
| | (S)-pyrrolidine-1,2-dicarboxylate; |
| 167 | 5-ethynyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-yl (2-methoxyethyl)carbamate; |
| 168 | tert-butyl |
| | (5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl) |
| | ethane-1,2-diylbis(methylcarbamate); |
| 169 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl |
| | methyl(2-(N-methylacetamido)ethyl)carbamate; |
| 170 | isopropyl |
| | (ethoxy((5-ethynyl-6-fluoro-4-(8-fluoro-2-(((2R,7aS)-2- |
| | fluorotetrahydro-1H-pyrrolizin-7a(5H)-yl)methoxy)-4-(1,4- |
| | oxazepan-4-yl)pyrido[4,3-d]pyrimidin-7-yl)naphthalen-2-yl)oxy) |
| | phosphoryl)-L-alaninate; |
| 171 | 1((2-ethylhexyl)oxy)-3-((5-ethynyl-6-fluoro-4-(8-fluoro-2- |
| | (((2R,7aS)-2-fluorotetrahydro-1H-pyrrolizin-7a(5H)-yl)methoxy)- |
| | 4-(1,4-oxazepan-4-yl)pyrido[4,3-d]pyrimidin-7-yl)naphthalen-2- |
| | yl)oxy)propan-2-ol; |
| 172 | 4-(7-(8-ethynyl-7-fluoro-3-((1-methyl-2-nitro-1H-imidazol-4-yl) |
| | methoxy)naphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)- |
| | 1,4-oxazepan; |
| 173 | 2-(tert-butyl) |
| | 1-(5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl) piperidine-1,2-dicarboxylate; |
| 174 | 4-(((5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl)oxy)methyl)phenol; |
| 175 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl isopropylcarbamate; |
| 176 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl morpholine-4-carboxylate; |
| 177 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl |
| | (((S)-tetrahydrofuran-2-yl)methyl)carbamate; |
| 178 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl |
| | (S)-2-(methoxymethyl)pyrrolidine-1-carboxylate; |
| 179 | 4-(7-(3-(ethoxymethoxy)-8-ethynyl-7-fluoronaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepane; |
| 180 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)quinazolin- |
| | 7-yl)naphthalen-2-ol; |
| 181 | 4-(6,8-difluoro-2-(((2R,7aS)-2-fluorotetrahydro-1H-pyrrolizin- |
| | 7a(5H)-yl)methoxy)-4-(1,4-oxazepan-4-yl)quinazolin-7-yl)- |
| | 5-ethynyl-6-fluoronaphthalen-2-ol; |
| 182 | (P)-4-(6,8-difluoro-2-(((2R,7aS)-2-fluorotetrahydro-1H-pyrrolizin- |
| | 7a(5H)-yl)methoxy)-4-(1,4-oxazepan-4-yl)quinazolin-7-yl)-5- |
| | ethynyl-6-fluoronaphthalen-2-ol; |
| 183 | (M)-4-(6,8-difluoro-2-(((2R,7aS)-2-fluorotetrahydro-1H-pyrrolizin- |
| | 7a(5H)-yl)methoxy)-4-(1,4-oxazepan-4-yl)quinazolin-7-yl)-5- |
| | ethynyl-6-fluoronaphthalen-2-ol; |
| 184 | 5-ethynyl-6-fluoro-4-(8-fluoro-2-(((2R,7aS)-2-fluorotetrahydro- |
| | 1H-pyrrolizin-7a(5H)-yl)methoxy)-4-(2-methyl-1,4-oxazepan-4- |
| | yl)pyrido[4,3-d]pyrimidin-7-yl)naphthalen-2-ol; |
| 185 | 4-(7-(benzo[b]thiophen-3-yl)-8-fluoro-2-(((2R,7aS)-2- |
| | fluorotetrahydro-1H-pyrrolizin-7a(5H)-yl)methoxy)pyrido |
| | [4,3-d]pyrimidin-4-yl)-1,4-oxazepane; |
| 186 | 4-(2-((4-acryloyl-1-methylpiperazin-2-yl)methoxy)-8-fluoro-4- |
| | (1,4-oxazepan-4-yl)pyrido[4,3-d]pyrimidin-7-yl)-5-ethynyl-6- |
| | fluoronaphthalen-2-yl acrylate; |
| 187 | 4-(7-(3-(difluoromethyl)-8-ethynyl-7-fluoronaphthalen-1-yl)-8- |
| | fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepane; |
| 188 | (4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d] |
| | pyrimidin-7-yl)naphthalen-2-yl)methanol; |
| 189 | 4-(8-fluoro-2-(((2R,7aS)-2-fluorotetrahydro-1H-pyrrolizin-7a(5H)- |
| | yl)methoxy)-7-(1H-indol-3-yl)pyrido[4,3-d]pyrimidin-4-yl)- |
| | 1,4-oxazepane; |
| 190 | 4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepan-6-ol; |
| 191 | 4-(8-fluoro-2-(((2R,7aS)-2-fluorotetrahydro-1H-pyrrolizin-7a(5H)- |
| | yl)methoxy)-7-(2-methoxy-5-(trifluoromethyl)phenyl)pyrido[4,3-d] |
| | pyrimidin-4-yl)-1,4-oxazepane; |
| 192 | 4-(4-(2-oxa-6-azabicyclo[5.1.0]octan-6-yl)-8-fluoro-2-((S)-1-((S)- |
| | 1-methylpyrrolidin-2-yl)ethoxy)pyrido[4,3-d]pyrimidin-7-yl)-5- |
| | ethynyl-6-fluoronaphthalen-2-ol; |
| 193 | 3-chloro-5-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)-4-(trifluoromethyl)phenol; |
| 194 | 5-ethynyl-6-fluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-yl cyclopropylcarbamate; |
| 195 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-ethynyl-6-fluoronaphthalen-2-ol; |
| 196 | 5-ethynyl-6-fluoro-4-(8-fluoro-4-(6-methylene-1,4-oxazepan-4-yl)- |
| | 2-(((2R,7aS)-2-(methylthio)tetrahydro-1H-pyrrolizin-7a(5H)-yl) |
| | methoxy)pyrido[4,3-d]pyrimidin-7-yl)naphthalen-2-ol; |
| 197 | 4-(4-(6,6-difluoro-1,4-oxazepan-4-yl)-8-fluoro-2-(((2R,7aS)-2- |
| | methoxytetrahydro-1H-pyrrolizin-7a(5H)-yl)methoxy)pyrido |
| | [4,3-d]pyrimidin-7-yl)-5-ethynyl-6-fluoronaphthalen-2-ol; |
| 198 | 4-(4-(2-oxa-6-azabicyclo[5.1.0]octan-6-yl)-8-fluoro-2-(((2R,7aS)- |
| | 2-methoxytetrahydro-1H-pyrrolizin-7a(5H)-yl)methoxy)pyrido |
| | [4,3-d]pyrimidin-7-yl)-5-ethynyl-6-fluoronaphthalen-2-ol; |
| 199 | 2-(((3R,7aR)-7a-(((7-(8-ethynyl-7-fluoro-3-hydroxynaphthalen-1- |
| | yl)-8-fluoro-4-(1,4-oxazepan-4-yl)pyrido[4,3-d]pyrimidin-2-yl)oxy) |
| | methyl)hexahydro-1H-pyrrolizin-3-yl)methoxy)-N,N- |
| | dimethylacetamide; |
| 200 | 5-ethynyl-6-fluoro-4-(8-fluoro-2-(((2R,7aS)-2-fluoro-2- |
| | methyltetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 201 | 5-ethynyl-6-fluoro-4-(8-fluoro-2-(((2R,7aS)-2-fluoro-2- |
| | methoxytetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 202 | 5-ethynyl-4-(4-(6-ethynyl-1,4-oxazepan-4-yl)-8-fluoro-2-(((2R,7aS)- |
| | 2-fluorotetrahydro-1H-pyrrolizin-7a(5H)methoxy)pyrido[4,3-d] |
| | pyrimidin-7-yl)-6-fluoronaphthalen-2-ol; |
| 203 | 5-ethynyl-6-fluoro-4-(8-fluoro-4-((S)-6-fluoro-6-methyl-1,4- |
| | oxazepan-4-yl)-2-(((2R,7aS)-2-fluorotetrahydro-1H-pyrrolizin- |
| | 7a(5H)-yl)methoxy)pyrido[4,3-d]pyrimidin-7-yl)naphthalen-2-ol; |
| 204 | 5-ethynyl-6-fluoro-4-(8-fluoro-4-((R)-6-fluoro-6-methyl-1,4- |
| | oxazepan-4-yl)-2-(((2R,7aS)-2-fluorotetrahydro-1H-pyrrolizin- |
| | 7a(5H)-yl)methoxy)pyrido[4,3-d]pyrimidin-7-yl)naphthalen-2-ol; |
| 205 | 1-(4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepan-6-yl)-1,4-oxazepan- |
| | 6-yl)-2,2,2-trifluoroethan-1-one; |
| 206 | 5-ethynyl-6-fluoro-4-(8-fluoro-2-(((2R,7aS)-2-fluorotetrahydro- |
| | 1H-pyrrolizin-7a(5H)-yl)methoxy)-4-(6-(1-hydroxyethyl)-1,4- |
| | oxazepan-4-yl)pyrido[4,3-d]pyrimidin-7-yl)naphthalen-2-ol; |
| 207 | cyclopropyl(4-(7-(8-ethynyl-7-fluoro-3-hydroxynaphthalen-1-yl)- |
| | 8-fluoro-2-(((2R,7aS)-2-fluorotetrahydro-1H-pyrrolizin-7a(5H)-yl) |
| | methoxy)pyrido[4,3-d]pyrimidin-4-yl)-1,4-oxazepan-6-yl) |
| | methanone; |
| 208 | 1-(4-(7-(8-ethynyl-7-fluoro-3-hydroxynaphthalen-1-yl)-8-fluoro-2- |
| | (((2R,7aS)-2-fluorotetrahydro-1H-pyrrolizin-7a(5H)-yl)methoxy) |
| | pyrido[4,3-d]pyrimidin-4-yl)-1,4-oxazepan-6-yl)ethan-1-one; |
| 209 | 5-ethynyl-6-fluoro-4-(8-fluoro-2-(((2R,7aS)-2-fluorotetrahydro- |
| | 1H-pyrrolizin-7a(5H)-yl)methoxy)-4-(6-methoxy-3,6-dimethyl- |
| | 1,4-oxazepan-4-yl)pyrido[4,3-d]pyrimidin-7-yl)naphthalen-2-ol; |
| 210 | 5-ethynyl-6-fluoro-4-(8-fluoro-4-(6-fluoro-3-methyl-6-vinyl-1,4- |
| | oxazepan-4-yl)-2-(((2R,7aS)-2-fluorotetrahydro-2H-pyrrolizin- |
| | 7a(5H)-yl)methoxy)pyrido[4,3-d]pyrimidin-7-yl)naphthalen-2-ol; |
| 211 | 4-(4-(6-ethyl-3-methyl-1,4-oxazepan-4-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 212 | 4-(4-(6-cyclopropyl-3-methyl-1,4-oxazepan-4-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 213 | 5-ethynyl-6-fluoro-4-(8-fluoro-4-(6-(fluoromethyl)-3-methyl-1,4- |
| | oxazepan-4-yl)-2-(((2R,7aS)-2-fluorotetrahydro-1H-pyrrolizin- |
| | 7a(5H)-yl)methoxy)pyrido[4,3-d]pyrimidin-7-yl)naphthalen-2-ol; |
| 214 | (R)-4-(7-(8-ethynyl-7-fluoro-3-hydroxynaphthalen-1-yl)-8-fluoro- |
| | 2-(((2R,7aS)-2-fluorotetrahydro-1H-pyrrolizin-7a(5H)-yl) |
| | methoxy)pyrido[4,3-d]pyrimidin-4-yl)-6-methyl-1,4-oxazepan-6-ol; |
| 215 | (S)-4-(7-(8-ethynyl-7-fluoro-3-hydroxynaphthalen-1-yl)-8-fluoro- |
| | 2-(((2R,7aS)-2-fluorotetrahydro-1H-pyrrolizin-7a(5H)-yl)methoxy) |
| | pyrido[4,3-d]pyrimidin-4-yl)-6-methyl-1,4-oxazepan-6-ol; |
| 216 | 5-ethynyl-6-fluoro-4-(8-fluoro-2-(((2R,7aS)-2-fluorotetrahydro- |
| | 1H-pyrrolizin-7a(5H)-yl)methoxy)-4-(2-oxa-6-azaspiro[bicyclo |
| | [5.1.0]octane-4,1′-cyclopropan]-6-yl)pyrido[4,3-d]pyrimidin-7-yl) |
| | naphthalen-2-ol; |
| 217 | 5-ethynyl-6-fluoro-4-(8-fluoro-2-(((2R,7aS)-2-fluorotetrahydro-1H- |
| | pyrrolizin-7a(5H)-yl)methoxy)-4-(4-methyl-2-oxa-6-azabicyclo |
| | [5.1.0]octan-6-yl)pyrido[4,3-d]pyrimidin-7-yl)naphthalen-2-ol; |
| 218 | 5-ethynyl-6-fluoro-4-(8-fluoro-2-(((2R,7aS)-2-fluoro-2- |
| | methyltetrahydro-1H-pyrrolizin-7a(5H)-yl)methoxy)-4-(4-methyl- |
| | 2-oxa-6-azabicyclo[5.1.0]octan-6-yl)pyrido[4,3-d]pyrimidin-7-yl) |
| | naphthalen-2-ol; |
| 219 | 4-(4-(4-cyclopropyl-2-oxa-6-azabicyclo[5.1.0]octan-6-yl)-8-fluoro- |
| | 2-(((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 220 | 4-(4-(4-cyclopropyl-2-oxa-6-azabicyclo[5.1.0]octan-6-yl)-8- |
| | fluoro-2-(((2R,7aS)-2-fluoro-2-methyltetrahydro-1H-pyrrolizin- |
| | 7a(5H)-yl)methoxy)pyrido[4,3-d]pyrimidin-7-yl)-5-ethynyl-6- |
| | fluoronaphthalen-2-ol; |
| 221 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethyl-6-fluoronaphthalen-2-ol; |
| 222 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethyl-6-fluoronaphthalen-2-ol; |
| 223 | 4-(4-((1S,7R)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 224 | 4-(4-((1R,7S)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 225 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methyl) |
| | thio)pyrido[4,3-d]pyrimidin-7-yl)-5-ethynyl-6-fluoronaphthalen- |
| | 2-ol; |
| 226 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methyl) |
| | thio)pyrido[4,3-d]pyrimidin-7-yl)-5-ethynyl-6-fluoronaphthalen- |
| | 2-ol; |
| 227 | 4-(4-((1S,7R)-2-oxa-6-azabicyclo[5.1.0]octan-6-yl)-8-fluoro-2- |
| | (((2R,7aS)-2-fluoro-2-methyltetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 228 | 4-(4-((1R,7S)-2-oxa-6-azabicyclo[5.1.0]octan-6-yl)-8-fluoro-2- |
| | (((2R,7aS)-2-fluoro-2-methyltetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 229 | 4-(4-((1S,7R)-2-oxa-6-azabicyclo[5.1.0]octan-6-yl)-8-fluoro-2- |
| | (((2R,7aS)-2-methoxytetrahydro-1H-pyrrolizin-7a(5H)-yl)methoxy) |
| | pyrido[4,3-d]pyrimidin-7-yl)-5-ethynyl-6-fluoronaphthalen-2-ol; |
| 230 | 4-(4-((2R,7S)-2-oxa-6-azabicyclo[5.1.0]octan-6-yl)-8-fluoro-2- |
| | (((2R,7aS)-2-methoxytetrahydro-1H-pyrrolizin-7a(5H)-yl)methoxy) |
| | pyrido[4,3-d]pyrimidin-7-yl)-5-ethynyl-6-fluoronaphthalen-2-ol; |
| 231 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy- |
| | d2)pyrido[4,3-d]pyrimidin-7-yl)-5-ethynyl-6-fluoronaphthalen-2-ol; |
| 232 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy-d2) |
| | pyrido[4,3-d]pyrimidin-7-yl)-5-ethynyl-6-fluoronaphthalen-2-ol; |
| 233 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-5,5-d2) |
| | methoxy-d2)pyrido[4,3-d]pyrimidin-7-yl)-5-ethynyl-6- |
| | fluoronaphthalen-2-ol; |
| 234 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-5,5-d2) |
| | methoxy-d2)pyrido[4,3-d]pyrimidin-7-yl)-5-ethynyl-6- |
| | fluoronaphthalen-2-ol; |
| 235 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-2,5,5-d3) |
| | methoxy-d2)pyrido[4,3-d]pyrimidin-7-yl)-5-ethynyl-6- |
| | fluoronaphthalen-2-ol; |
| 236 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-2,5,5-d3) |
| | methoxy-d2)pyrido[4,3-d]pyrimidin-7-yl)-5-ethynyl-6- |
| | fluoronaphthalen-2-ol; |
| 237 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy)pyrido[4,3-d]pyrimidin-7-yl)-5-ethynyl-6- |
| | fluoronaphthalen-2-ol; |
| 238 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy)pyrido[4,3-d]pyrimidin-7-yl)-5-ethynyl-6-fluoronaphthalen- |
| | 2-ol; |
| 239 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-methoxypyrido[4,3-d]pyrimidin-7-yl)-5-ethynyl-6- |
| | fluoronaphthalen-2-ol; |
| 240 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-methoxypyrido[4,3-d]pyrimidin-7-yl)-5-ethynyl-6- |
| | fluoronaphthalen-2-ol; |
| 241 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-(methylamino)pyrido[4,3-d]pyrimidin-7-yl)-5-ethynyl-6- |
| | fluoronaphthalen-2-ol; |
| 242 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-(methylamino)pyrido[4,3-d]pyrimidin-7-yl)-5-ethynyl-6- |
| | fluoronaphthalen-2-ol; |
| 243 | 4-(4-((1S,7R)-2-oxa-6-azabicyclo[5.1.0]octan-6-yl)-5-ethoxy-8- |
| | fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6- |
| | fluoronaphthalen-2-ol; |
| 244 | 4-(4-((1R,7S)-2-oxa-6-azabicyclo[5.1.0]octan-6-yl)-5-ethoxy-8- |
| | fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6- |
| | fluoronaphthalen-2-ol; |
| 245 | 4-(4-((1S,7R)-2-oxa-6-azabicyclo[5.1.0]octan-6-yl)-5-cyclopropoxy- |
| | 8-fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 246 | 4-(4-((1R,7S)-2-oxa-6-azabicyclo[5.1.0]octan-6-yl)-5-cyclopropoxy- |
| | 8-fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 247 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-methylpyrido[4,3-d]pyrimidin-7-yl)-5-ethynyl-6- |
| | fluoronaphthalen-2-ol; |
| 248 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-methylpyrido[4,3-d]pyrimidin-7-yl)-5-ethynyl-6-fluoronaphthalen- |
| | 2-ol; |
| 249 | 4-(4-((1S,7R)-2-oxa-6-azabicyclo[5.1.0]octan-6-yl)-5-ethyl-8- |
| | fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 250 | 4-(4-((1R,7S)-2-oxa-6-azabicyclo[5.1.0]octan-6-yl)-5-ethyl-8- |
| | fluoro-2-(((2R,7S)-2-fluorotetrahydro-1H-pyrrolizin-7a(5H)-yl) |
| | methoxy)pyrido[4,3-d]pyrimidin-7-yl)-5-ethynyl-6- |
| | fluoronaphthalen-2-ol; |
| 251 | 4-(4-((1S,7R)-2-oxa-6-azabicyclo[5.1.0]octan-6-yl)-5-cyclopropyl- |
| | 8-fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 252 | 4-(4-((1R,7S)-2-oxa-6-azabicyclo[5.1.0]octan-6-yl)-5-cyclopropyl- |
| | 8-fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 253 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-(methylthio)pyrido[4,3-d]pyrimidin-7-yl)-5-ethynyl-6- |
| | fluoronaphthalen-2-ol; |
| 254 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-(methylthio)pyrido[4,3-d]pyrimidin-7-yl)-5-ethynyl-6- |
| | fluoronaphthalen-2-ol; |
| 255 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-isopropoxypyrido[4,3-d]pyrimidin-7-yl)-5-ethynyl-6- |
| | fluoronaphthalen-2-ol; |
| 256 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy)- |
| | 5-isopropoxypyrido[4,3-d]pyrimidin-7-yl)-5-ethynyl-6- |
| | fluoronaphthalen-2-ol; |
| 257 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6,7-difluoronaphthalen- |
| | 2-ol; |
| 258 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6,7-difluoronaphthalen- |
| | 2-ol; |
| 259 | 5-ethynyl-6-fluoro-4-(8-fluoro-2-(((2R,7aS)-2-fluorotetrahydro-1H- |
| | pyrrolizin-7a(5H)-yl)methoxy)-4-(7-methyl-5-oxa-8-azaspiro[2.6] |
| | nonan-8-yl)pyrido[4,3-d]pyrimidin-7-yl)naphthalen-2-ol; |
| 260 | 4-(4-(2,3-dihydropyrido[3,2-f][1,4]oxazepin-4(5H)-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 261 | 5-ethynyl-6-fluoro-4-(8-fluoro-4-(4-fluoro-2-oxa-6-azabicyclo |
| | [5.2.0]nonan-6-yl)-2-(((2R,7aS)-2-fluorotetrahydro-1H-pyrrolizin- |
| | 7a(5H)-yl)methoxy)pyrido[4,3-d]pyrimidin-7-yl)naphthalen-2-ol; |
| 262 | 5-ethynyl-6-fluoro-4-(8-fluoro-2-(((2R,7aS)-2-fluoro-2- |
| | methyltetrahydro-1H-pyrrolizin-7a(5H)-yl)methoxy)-4-(4-fluoro- |
| | 2-oxa-6-azabicyclo[5.2.0]nonan-6-yl)pyrido[4,3-d]pyrimidin-7-yl) |
| | naphthalen-2-ol; |
| 263 | 5-ethynyl-6-fluoro-4-(8-fluoro-2-(((2R,7aS)-2-fluorotetrahydro-1H- |
| | pyrrolizin-7a(5H)-yl)methoxy)-4-(4-methyl-2-oxa-6-azabicyclo |
| | [5.2.0]nonan-6-yl)pyrido[4,3-d]pyrimidin-7-yl)naphthalen-2-ol; |
| 264 | 5-ethynyl-6-fluoro-4-(8-fluoro-2-(((2R,7aS)-2-fluoro-2- |
| | methyltetrahydro-1H-pyrrolizin-7a(5H)-yl)methoxy)-4-(4-methyl- |
| | 2-oxa-6-azabicyclo[5.2.0]nonan-6-yl)pyrido[4,3-d]pyrimidin-7-yl) |
| | naphthalen-2-ol; |
| 265 | 4-(4-(2-oxa-5-azabicyclo[5.2.0]nonan-5-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 266 | 4-(4-(5-oxa-2-azabicyclo[5.2.0]nonan-2-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 267 | 5-ethyl-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 268 | 6-fluoro-4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)-5-(methylsulfinyl)naphthalen-2-ol; |
| 269 | 5-(cyclopropylethynyl)-6-fluoro-4-(8-fluoro-2-(((2R,7aS)-2- |
| | fluorotetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 270 | 5-(cyclopropylmethyl)-6-fluoro-4-(8-fluoro-2-(((2R,7aS)-2- |
| | fluorotetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 271 | 4-(4-(2-oxa-6-azabicyclo[5.2.0]nonan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 272 | 4-(4-(2-oxa-5-azabicyclo[5.1.0]octan-5-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6-fluoronaphthalen-2-ol; |
| 273 | 5-ethyl-6,7-difluoro-4-(8-fluoro-2-(((2R,7aS)-2-fluoro-2- |
| | methyltetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 274 | 5-ethyl-6-fluoro-4-(8-fluoro-2-(((2R,7aS)-2-fluoro-2- |
| | methyltetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 275 | 5-ethynyl-6,7-difluoro-4-(8-fluoro-2-(((2R,7aS)-2-fluoro-2- |
| | methyltetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 276 | 5,6-difluoro-4-(8-fluoro-2-(((2R,7aS)-2-fluoro-2-methyltetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 277 | 5-bromo-6-fluoro-4-(8-fluoro-2-(((2R,7aS)-2-fluoro-2- |
| | methyltetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 278 | 5-ethynyl-4-(8-fluoro-2-(((2R,7aS)-2-fluoro-2-methyltetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 279 | 5-chloro-6-fluoro-4-(8-fluoro-2-(((2R,7aS)-2-fluoro-2- |
| | methyltetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 280 | 4-(8-fluoro-2-(((2R,7aS)-2-fluoro-2-methyltetrahydro-1H- |
| | pyrrolizin-7a(5H)-yl)methoxy)-4-(1,4-oxazepan-4-yl)pyrido[4,3-d] |
| | pyrimidin-7-yl)-5-(methylthio)naphthalen-2-ol; |
| 281 | 5-ethyl-4-(8-fluoro-2-(((2R,7aS)-2-fluoro-2-methyltetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 282 | 5-ethyl-6,7-difluoro-4-(8-fluoro-2-(((2R,7aS)-2-fluorotetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 283 | 5-bromo-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 284 | 5-chloro-6-fluoro-4-(8-fluoro-2-(((2R,7aS)-2-fluorotetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 285 | 4-(8-fluoro-2-(((2R,7aS)-2-fluorotetrahydro-1H-pyrrolizin-7a(5H)- |
| | yl)methoxy)-4-(1,4-oxazepan-4-yl)pyrido[4,3-d]pyrimidin-7-yl)-5- |
| | (methylthio)naphthalen-2-ol; |
| 286 | 5-ethyl-4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)naphthalen-2-ol; |
| 287 | 5-ethyl-6,7-difluoro-4-(8-fluoro-2-(((2R,7aS)-2-methoxytetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 288 | 5-ethyl-6-fluoro-4-(8-fluoro-2-(((2R,7aS)-2-methoxytetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 289 | 5-ethynyl-6,7-difluoro-4-(8-fluoro-2-(((2R,7aS)-2- |
| | methoxytetrahydro-1H-pyrrolizin-7a(5H)-yl)methoxy)-4-(1,4- |
| | oxazepan-4-yl)pyrido[4,3-d]pyrimidin-7-yl)naphthalen-2-ol; |
| 290 | 5,6-difluoro-4-(8-fluoro-2-(((2R,7aS)-2-methoxytetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 291 | 5-bromo-6-fluoro-4-(8-fluoro-2-(((2R,7aS)-2-methoxytetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 292 | 5-ethynyl-4-(8-fluoro-2-(((2R,7aS)-2-methoxytetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 293 | 5-chloro-6-fluoro-4-(8-fluoro-2-(((2R,7aS)-2-methoxytetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 294 | 4-(8-fluoro-2-(((2R,7aS)-2-methoxytetrahydro-1H-pyrrolizin- |
| | 7a(5H)-yl)methoxy)-4-(1,4-oxazepan-4-yl)pyrido[4,3-d] |
| | pyrimidin-7-yl)-5-(methylthio)naphthalen-2-ol; |
| 295 | 5-ethyl-4-(8-fluoro-2-(((2R,7aS)-2-methoxytetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 296 | 5-ethyl-6,7-difluoro-4-(8-fluoro-2-(((2R,7aS)-2-(methylthio) |
| | tetrahydro-1H-pyrrolizin-7a(5H)-yl)methoxy)-4-(1,4-oxazepan- |
| | 4-yl)pyrido[4,3-d]pyrimidin-7-yl)naphthalen-2-ol; |
| 297 | 5-ethyl-6-fluoro-4-(8-fluoro-2-(((2R,7aS)-2-(methylthio)tetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 298 | 5-ethynyl-6,7-difluoro-4-(8-fluoro-2-(((2R,7aS)-2-(methylthio) |
| | tetrahydro-1H-pyrrolizin-7a(5H)-yl)methoxy)-4-(1,4-oxazepan-4- |
| | yl)pyrido[4,3-d]pyrimidin-7-yl)naphthalen-2-ol; |
| 299 | 5,6-difluoro-4-(8-fluoro-2-(((2R,7aS)-2-(methylthio)tetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 300 | 5-ethynyl-6-fluoro-4-(8-fluoro-2-(((2R,7aS)-2-(methylthio) |
| | tetrahydro-1H-pyrrolizin-7a(5H)-yl)methoxy)-4-(1,4-oxazepan-4- |
| | yl)pyrido[4,3-d]pyrimidin-7-yl)naphthalen-2-ol; |
| 301 | 5-bromo-6-fluoro-4-(8-fluoro-2-(((2R,7aS)-2-(methylthio)tetrahydro- |
| | 1H-pyrrolizin-7a(5H)-yl)methoxy)-4-(1,4-oxazepan-4-yl)pyrido |
| | [4,3-d]pyrimidin-7-yl)naphthalen-2-ol; |
| 302 | 5-ethynyl-4-(8-fluoro-2-(((2R,7aS)-2-(methylthio)tetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 303 | 5-chloro-6-fluoro-4-(8-fluoro-2-(((2R,7aS)-2-(methylthio) |
| | tetrahydro-1H-pyrrolizin-7a(5H)-yl)methoxy)-4-(1,4-oxazepan- |
| | 4-yl)pyrido[4,3-d]pyrimidin-7-yl)naphthalen-2-ol; |
| 304 | 4-(8-fluoro-2-(((2R,7aS)-2-(methylthio)tetrahydro-1H-pyrrolizin- |
| | 7a(5H)-yl)methoxy)-4-(1,4-oxazepan-4-yl)pyrido[4,3-d]pyrimidin- |
| | 7-yl)-5-(methylthio)naphthalen-2-ol; |
| 305 | 5-ethyl-4-(8-fluoro-2-(((2R,7aS)-2-(methylthio)tetrahydro-1H- |
| | pyrrolizin-7a(5H)-yl)methoxy)-4-(1,4-oxazepan-4-yl)pyrido[4,3-d] |
| | pyrimidin-7-yl)naphthalen-2-ol; |
| 306 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-ethyl-6,7-difluoronaphthalen-2-ol; |
| 307 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-ethyl-6-fluoronaphthalen-2-ol; |
| 308 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-ethynyl-6,7-difluoronaphthalen-2-ol; |
| 309 | 4-(4-(2-oxa-6-azabicyclo[5.1.0]octan-6-yl)-8-fluoro-2-(((2R,7aS)-2- |
| | fluoro-2-methyltetrahydro-1H-pyrrolizin-7a(5H)-yl)methoxy) |
| | pyrido[4,3-d]pyrimidin-7-yl)-5,6-difluoronaphthalen-2-ol; |
| 310 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-ethynyl-6-fluoronaphthalen-2-ol; |
| 311 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-bromo-6-fluoronaphthalen-2-ol; |
| 312 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-ethynylnaphthalen-2-ol; |
| 313 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-chloro-6-fluoronaphthalen-2-ol; |
| 314 | 4-(4-(2-oxa-6-azabicyclo[5.1.0]octan-6-yl)-8-fluoro-2-(((2R,7aS)-2- |
| | fluoro-2-methyltetrahydro-1H-pyrrolizin-7a(5H)-yl)methoxy)pyrido |
| | [4,3-d]pyrimidin-7-yl)-5-(methylthio)naphthalen-2-ol; |
| 315 | 4-(4-(2-oxa-6-azabicyclo[5.1.0]octan-6-yl)-8-fluoro-2-(((2R,7aS)- |
| | 2-fluoro-2-methyltetrahydro-1H-pyrrolizin-7a(5H)-yl)methoxy) |
| | pyrido[4,3-d]pyrimidin-7-yl)-5-ethylnaphthalen-2-ol; |
| 316 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethyl-6,7-difluoronaphthalen-2-ol; |
| 317 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethyl-6-fluoronaphthalen-2-ol; |
| 318 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethynyl-6,7-difluoronaphthalen-2-ol; |
| 319 | 4-(4-(2-oxa-6-azabicyclo[5.1.0]octan-6-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-5,6-difluoronaphthalen-2-ol; |
| 320 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-bromo-6-fluoronaphthalen-2-ol; |
| 321 | 4-(4-(2-oxa-6-azabicyclo[5.1.0]octan-6-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-5-ethynylnaphthalen-2-ol; |
| 322 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-chloro-6-fluoronaphthalen-2-ol; |
| 323 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-(methylthio)naphthalen-2-ol; |
| 324 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy)pyrido[4,3-d] |
| | pyrimidin-7-yl)-5-ethylnaphthalen-2-ol; |
| 325 | 4-(4-(2-oxa-6-azabicyclo[5.1.0]octan-6-yl)-8-fluoro-2-(((2R,7aS)-2- |
| | methoxytetrahydro-1H-pyrrolizin-7a(5H)-yl)methoxy)pyrido[4,3-d] |
| | pyrimidin-7-yl)-5-ethyl-6,7-difluoronaphthalen-2-ol; |
| 326 | 4-(4-(2-oxa-6-azabicyclo[5.1.0]octan-6-yl)-8-fluoro-2-(((2R,7aS)- |
| | 2-methoxytetrahydro-1H-pyrrolizin-7a(5H)-yl)methoxy)pyrido |
| | [4,3-d]pyrimidin-7-yl)-5-ethyl-6-fluoronaphthalen-2-ol; |
| 327 | 4-(4-(2-oxa-6-azabicyclo[5.1.0]octan-6-yl)-8-fluoro-2-(((2R,7aS)- |
| | 2-methoxytetrahydro-1H-pyrrolizin-7a(5H)-yl)methoxy)pyrido |
| | [4,3-d]pyrimidin-7-yl)-5-ethynyl-6,7-difluoronaphthalen-2-ol; |
| 328 | 4-(4-(2-oxa-6-azabicyclo[5.1.0]octan-6-yl)-8-fluoro-2-(((2R,7aS)-2- |
| | methoxytetrahydro-1H-pyrrolizin-7a(5H)-yl)methoxy)pyrido[4,3-d] |
| | pyrimidin-7-yl)-5,6-difluoronaphthalen-2-ol; |
| 329 | 4-(4-(2-oxa-6-azabicyclo[5.1.0]octan-6-yl)-8-fluoro-2-(((2R,7aS)- |
| | 2-methoxytetrahydro-1H-pyrrolizin-7a(5H)-yl)methoxy)pyrido |
| | [4,3-d]pyrimidin-7-yl)-5-bromo-6-fluoronaphthalen-2-ol; |
| 330 | 4-(4-(2-oxa-6-azabicyclo[5.1.0]octan-6-yl)-8-fluoro-2-(((2R,7aS)- |
| | 2-methoxytetrahydro-1H-pyrrolizin-7a(5H)-yl)methoxy)pyrido |
| | [4,3-d]pyrimidin-7-yl)-5-ethynylnaphthalen-2-ol; |
| 331 | 4-(4-(2-oxa-6-azabicyclo[5.1.0]octan-6-yl)-8-fluoro-2-(((2R,7aS)-2- |
| | methoxytetrahydro-1H-pyrrolizin-7a(5H)-yl)methoxy)pyrido[4,3-d] |
| | pyrimidin-7-yl)-5-chloro-6-fluoronaphthalen-2-ol; |
| 332 | 4-(4-(2-oxa-6-azabicyclo[5.1.0]octan-6-yl)-8-fluoro-2-(((2R,7aS)-2- |
| | methoxytetrahydro-1H-pyrrolizin-7a(5H)-yl)methoxy)pyrido[4,3-d] |
| | pyrimidin-7-yl)-5-(methylthio)naphthalen-2-ol; |
| 333 | 4-(4-(2-oxa-6-azabicyclo[5.1.0]octan-6-yl)-8-fluoro-2-(((2R,7aS)-2- |
| | methoxytetrahydro-1H-pyrrolizin-7a(5H)-yl)methoxy)pyrido[4,3-d] |
| | pyrimidin-7-yl)-5-ethylnaphthalen-2-ol; |
| 334 | 4-(4-(2-oxa-6-azabicyclo[5.1.0]octan-6-yl)-8-fluoro-2-(((2R,7aS)-2- |
| | (methylthio)tetrahydro-1H-pyrrolizin-7a(5H)-yl)methoxy)pyrido |
| | [4,3-d]pyrimidin-7-yl)-5-ethyl-6,7-difluoronaphthalen-2-ol; |
| 335 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-ethyl-6-fluoronaphthalen-2-ol; |
| 336 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-ethynyl-6,7-difluoronaphthalen-2-ol; |
| 337 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5,6-difluoronaphthalen-2-ol; |
| 338 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-bromo-6-difluoronaphthalen-2-ol; |
| 339 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-ethynylnaphthalen-2-ol; |
| 340 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-chloro-6-fluoronaphthalen-2-ol; |
| 341 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-(methylthio)naphthalen-2-ol; |
| 342 | 4-(4-(2-oxa-6-azabicyclo[5.1.0]octan-6-yl)-8-fluoro-2-(((2R,7aS)- |
| | 2-(methylthio)tetrahydro-1H-pyrrolizin-7a(5H)-yl)methoxy)pyrido |
| | [4,3-d]pyrimidin-7-yl)-5-ethylnaphthalen-2-ol; |
| 343 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-2,5,5-d3) |
| | methoxy-d2)pyrido[4,3-d]pyrimidin-7-yl)-5-ethyl-6- |
| | fluoronaphthalen-2-ol; |
| 344 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-2,5,5-d3) |
| | methoxy-d2)pyrido[4,3-d]pyrimidin-7-yl)-5-ethynyl-6- |
| | fluoronaphthalen-2-ol; |
| 345 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-2-d) |
| | pyrido[4,3-d]pyrimidin-7-yl)-5-ethyl-6-fluoronaphthalen-2-ol; |
| 346 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-2-d) |
| | methoxy-d2)pyrido[4,3-d]pyrimidin-7-yl)-5-ethynyl-6- |
| | fluoronaphthalen-2-ol; |
| 347 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy- |
| | d2)pyrido[4,3-d]pyrimidin-7-yl)-5-ethyl-6-fluoronaphthalen-2-ol; |
| 348 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy- |
| | d2)pyrido[4,3-d]pyrimidin-7-yl)-5-ethynyl-6-fluoronaphthalen-2-ol; |
| 349 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-2-d)methoxy) |
| | pyrido[4,3-d]pyrimidin-7-yl)-5-ethyl-6-fluoronaphthalen-2-ol; |
| 350 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-2-d)methoxy) |
| | pyrido[4,3-d]pyrimidin-7-yl)-5-ethynyl-6-fluoronaphthalen-2-ol; |
| 351 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-5,5-d2) |
| | methoxy-d2)pyrido[4,3-d]pyrimidin-7-yl)-5-ethyl-8- |
| | fluoronaphthalen-2-ol; |
| 352 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl-5,5-d2) |
| | methoxy-d2)pyrido[4,3-d]pyrimidin-7-yl)-5-ethynyl-6- |
| | fluoronaphthalen-2-ol; |
| 353 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-2,5,5-d3) |
| | methoxy-d2)pyrido[4,3-d]pyrimidin-7-yl)-5-ethyl-6- |
| | fluoronaphthalen-2-ol; |
| 354 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy-d2)pyrido[4,3-d]pyrimidin-7-yl)-5-ethyl-6- |
| | fluoronaphthalen-2-ol; |
| 355 | 4-(4-((1S,7R)-2-oxa-6-azabicyclo[5.1.0]octan-6-yl)-8-fluoro-2- |
| | (((2R,7aS)-2-fluorotetrahydor-1H-pyrrolizin-7a(5H)-yl-2-d) |
| | methoxy-d2)pyrido[4,3-d]pyrimidin-7-yl)-5-ethynyl-6- |
| | fluoronaphthalen-2-ol; |
| 356 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy- |
| | d2)pyrido[4,3-d]pyrimidin-7-yl)-5-ethyl-6-fluoronaphthalen-2-ol; |
| 357 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy)pyrido[4,3-d]pyrimidin-7-yl)-5-ethyl-6- |
| | fluoronaphthalen-2-ol; |
| 358 | 4-(4-(((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl-5,5-d2) |
| | methoxy-d2)pyrido[4,3-d]pyrimidin-7-yl)-5-ethyl-6- |
| | fluoronaphthalen-2-ol; |
| 359 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-2,5,5-d3) |
| | methoxy-d2)pyrido[4,3-d]pyrimidin-7-yl)-5-ethyl-6- |
| | fluoronaphthalen-2-ol; |
| 360 | 4-(4-((1R,7aS)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy-d2)pyrido[4,3-d]pyrimidin-7-yl)-5-ethyl-6- |
| | fluoronaphthalen-2-ol; |
| 361 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy-d2)pyrido[4,3-d]pyrimidin-7-yl)-5-ethynyl-6- |
| | fluoronaphthalen-2-ol; |
| 362 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy- |
| | d2)pyrido[4,3-d]pyrimidin-7-yl)-5-ethyl-6-fluoronaphthalen-2-ol |
| 363 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-2-d) |
| | methoxy)pyrido[4,3-d]pyrimidin-7-yl)-5-ethyl-6-fluoronaphthalen- |
| | 2-ol; |
| 364 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl-5,5-d2) |
| | methoxy-d2)pyrido[4,3-d]pyrimidin-7-yl)-5-ethyl-6- |
| | fluoronaphthalen-2-ol; |
| 365 | 5-ethyl-6-fluoro-4-(8-fluoro-2-(((2R,7aS)-2-fluorotetrahydro-1H- |
| | pyrrolizin-7a(5H)-yl-2,5,5-d3)methoxy-d2)-4-(1,4-oxazepan-4-yl) |
| | pyrido[4,3-d]pyrimidin-7-yl)naphthalen-2-ol; |
| 366 | 5-ethynyl-6-fluoro-4-(8-fluoro-2-(((2R,7aS)-2-fluorotetrahydro-1H- |
| | pyrrolizin-7a(5H)-yl-2,5,5-d3)methoxy-d2)-4-(1,4-oxazepan-4-yl) |
| | pyrido[4,3-d]pyrimidin-7-yl)naphthalen-2-ol; |
| 367 | 5-ethyl-6-fluoro-4-(8-fluoro-2-(((2R,7aS)-2-fluorotetrahydro-1H- |
| | pyrrolizin-7a(5H)-yl-2-d)methoxy-d2)-4-(1,4-oxazepan-4-yl)pyrido |
| | [4,3-d]pyrimidin-7-yl)naphthalen-2-ol; |
| 368 | 5-ethynyl-6-fluoro-4-(8-fluoro-2-(((2R,7aS)-2-fluorotetrahydro-1H- |
| | pyrrolizin-7a(5H)-yl-2-d)methoxy-d2)-4-(1,4-oxazepan-4-yl) |
| | pyrido[4,3-d]pyrimidin-7-yl)naphthalen-2-ol; |
| 369 | 5-ethyl-6-fluoro-4-(8-fluoro-2-(((2R,7aS)-2-fluorotetrahydro-1H- |
| | pyrrolizin-7a(5H)-yl)methoxy-d2)-4-(1,4-oxazepan-4-yl)pyrido |
| | [4,3-d]pyrimidin-7-yl)naphthalen-2-ol; |
| 370 | 5-ethynyl-6-fluoro-4-(8-fluoro-2-(((2R,7aS)-2-fluorotetrahydro-1H- |
| | pyrrolizin-7a(5H)-yl)methoxy-d2)-4-(1,4-oxazepan-4-yl)pyrido |
| | [4,3-d]pyrimidin-7-yl)naphthalen-2-ol; |
| 371 | 5-ethyl-6-fluoro-4-(8-fluoro-2-(((2R,7aS)-2-fluorotetrahydro-1H- |
| | pyrrolizin-7a(5H)-yl-2-d)methoxy)-4-(1,4-oxazepan-4-yl)pyrido |
| | [4,3-d]pyrimidin-7-yl)naphthalen-2-ol; |
| 372 | 5-ethynyl-6-fluoro-4-(8-fluoro-2-(((2R,7aS)-2-fluorotetrahydro-1H- |
| | pyrrolizin-7a(5H)-yl-2-d)methoxy)-4-(1,4-oxazepan-4-yl)pyrido |
| | [4,3-d]pyrimidin-7-yl)naphthalen-2-ol; |
| 373 | 5-ethyl-6-fluoro-4-(8-fluoro-2-(((2R,7aS)-2-fluorotetrahydro-1H- |
| | pyrrolizin-7a(5H)-yl-5,5-d2)methoxy-d2)-4-(1,4-oxazepan-4-yl) |
| | pyrido[4,3-d]pyrimidin-7-yl)naphthalen-2-ol; |
| 374 | 5-ethynyl-6-fluoro-4-(8-fluoro-2-(((2R,7aS)-2-fluorotetrahydro-1H- |
| | pyrrolizin-7a(5H)-yl-5,5-d2)methoxy-d2)-4-(1,4-oxazepan-4-yl) |
| | pyrido[4,3-d]pyrimidin-7-yl)naphthalen-2-ol; |
| 375 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-2,5,5-d3)methoxy-d2) |
| | pyrido[4,3-d]pyrimidin-7-yl)-5-ethyl-6-fluoronaphthalen-2-ol; |
| 376 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-2,5,5-d3)methoxy-d2) |
| | pyrido[4,3-d]pyrimidin-7-yl)-5-ethynyl-6-fluoronaphthalen-2-ol; |
| 377 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-2-d)methoxy-d2) |
| | pyrido[4,3-d]pyrimidin-7-yl)-5-ethyl-6-fluoronaphthalen-2-ol; |
| 378 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-2-d)methoxy-d2) |
| | pyrido[4,3-d]pyrimidin-7-yl)-5-ethynyl-6-fluoronaphthalen-2-ol; |
| 379 | 4-(4-2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy-d2)pyrido |
| | [4,3-d]pyrimidin-7-yl)-5-ethyl-6-fluoronaphthalen-2-ol; |
| 380 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl)methoxy-d2)pyrido |
| | [4,3-d]pyrimidin-7-yl)-5-ethynyl-6-fluoronaphthalen-2-ol; |
| 381 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-2-d)methoxy)pyrido |
| | [4,3-d]pyrimidin-7-yl)-5-ethyl-6-fluoronaphthalen-2-ol; |
| 382 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-2-d)methoxy)pyrido |
| | [4,3-d]pyrimidin-7-yl)-5-ethynyl-6-fluoronaphthalen-2-ol; |
| 383 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-5,5-d2)methoxy-d2) |
| | pyrido[4,3-d]pyrimidin-7-yl)-5-ethyl-6-fluoronaphthalen-2-ol; |
| 384 | 4-(4-(2-oxa-6-azabicyclo[5.1.0]octan-6-yl)-8-fluoro-2-(((2R,7aS)- |
| | 2-fluorotetrahydro-1H-pyrrolizin-7a(5H)-yl-5,5-d2)methoxy-d2) |
| | pyrido[4,3-d]pyrimidin-7-yl)-5-ethynyl-6-fluoronaphthalen-2-ol; |
| 385 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-(methylthio)naphthalen-2-ol; |
| 386 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethyl-6,7-difluoronaphthalen-2-ol; |
| 387 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethyl-6-fluoronaphthalen-2-ol; |
| 388 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6,7-difluoronaphthalen-2-ol; |
| 389 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethynyl-6-fluoronaphthalen-2-ol; |
| 390 | 4-(4-(2-oxa-6-azabicyclo[5.1.0]octan-6-yl-8,8-d2)-8-fluoro-2- |
| | ((2R,7aS)-2-fluorotetrahydro-1H-pyrrolizin-7a(5H)-yl)methoxy) |
| | pyrido[4,3-d]pyrimidin-7-yl)-5-bromo-6-fluoronaphthalen-2-ol; |
| 391 | 4-(4-(2-oxa-6-azabicyclo[5.1.0]octan-6-yl-8,8-d2)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-6-fluoro-5-(methylthio)naphthalen- |
| | 2-ol; |
| 392 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-(methylthio)naphthalen- |
| | 2-ol; |
| 393 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethyl-6,7- |
| | difluoronaphthalen-2-ol; |
| 394 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethyl-6-fluoronaphthalen- |
| | 2-ol; |
| 395 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethynyl-6,7- |
| | difluoronaphthalen-2-ol; |
| 396 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethynyl-6-fluoronaphthalen- |
| | 2-ol; |
| 397 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-bromo-6- |
| | fluoronaphthalen-2-ol; |
| 398 | 4-(4-(2-oxa-6-azabicyclo[5.1.0]octan-6-yl-8,8-d2)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-6-fluoro-5-(methylthio) |
| | naphthalen-2-ol; |
| 399 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-(methylthio)naphthalen-2-ol; |
| 400 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethyl-6,7-difluoronaphthalen-2-ol; |
| 401 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-bromo-6-fluoronaphthalen-2-ol; |
| 402 | 4-(4-((1S,7R)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-6-fluoro-5-(methylthio)naphthalen- |
| | 2-ol; |
| 403 | 4-(4-((1S,7R)-2-oxa-6-azabicyclo[5.1.0]octan-6-yl)-8-fluoro-2- |
| | (((2R,7aS)-2-methylthio)tetrahydro-1H-pyrrolizin-7a(5H)-yl) |
| | methoxy)pyrido[4,3-d]pyrimidin-7-yl)-5-(methylthio)naphthalen- |
| | 2-ol; |
| 404 | 4-(4-((1S,7R)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethyl-6,7- |
| | difluoronaphthalen-2-ol; |
| 405 | 4-(4-((1S,7R)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethyl-6- |
| | fluoronaphthalen-2-ol; |
| 406 | 4-(4-((1S,7R)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethynyl-6,7- |
| | difluoronaphthalen-2-ol; |
| 407 | 4-(4-((1S,7R)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-bromo-6-fluoronaphthalen- |
| | 2-ol; |
| 408 | 4-(4-((1S,7R)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-6-fluoro-5-(methylthio) |
| | naphthalen-2-ol; |
| 409 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-(methylthio)naphthalen-2-ol; |
| 410 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-ethyl-6,7-difluoronaphthalen-2-ol; |
| 411 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-5-bromo-6-fluoronaphthalen-2-ol; |
| 412 | 4-(4-((1R,7S)-2-oxa-6-azabicyclo[5.1.0]octan-6-yl)-8-fluoro-2- |
| | (((2R,7aS)-2-fluorotetrahydro-1H-pyrrolizin-7a(5H)-yl)methoxy) |
| | pyrido[4,3-d]pyrimidin-7-yl)-6-fluoro-5-(methylthio)naphthalen- |
| | 2-ol; |
| 413 | 4-(4-((1R,7S)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-(methylthio)naphthalen- |
| | 2-ol; |
| 414 | 4-(4-((1R,7S)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethyl-6,7- |
| | difluoronaphthalen-2-ol; |
| 415 | 4-(4-((1R,7S)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethyl-6-fluoronaphthalen- |
| | 2-ol; |
| 416 | 4-(4-((1R,7S)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-ethynyl-6,7- |
| | difluoronaphthalen-2-ol; |
| 417 | 4-(4-((1R,7S)-2-oxa-6-azabicyclo[5.1.0]octan-6-yl)-8-fluoro-2- |
| | (((2R,7aS)-2-(methylthio)tetrahydro-1H-pyrrolizin-7a(5H)-yl) |
| | methoxy)pyrido[4,3-d]pyrimidin-7-yl)-5-bromo-6-fluoronaphthalen- |
| | 2-ol; |
| 418 | 4-(4-((1R,7S)-2-oxa-6-azabicyclo[5.1.0]octan-6-yl)-8-fluoro-2- |
| | ((2R,7aS)-2-(methylthio)tetrahydro-1H-pyrrolizin-7a(5H)-yl) |
| | methoxy)pyrido[4,3-d]pyrimidin-7-yl)-6-fluoro-5-(methylthio) |
| | naphthalen-2-ol; |
| 419 | 6-fluoro-4-(8-fluoro-2-(((2R,7aS)-2-fluorotetrahydro-1H-pyrrolizin- |
| | 7a(5H)-yl)methoxy)-4-(1,4-oxazepan-4-yl)pyrido[4,3-d]pyrimidin- |
| | 7-yl)-5-(methylthio)naphthalen-2-ol; |
| 420 | 6-fluoro-4-(8-fluoro-2-(((2R,7aS)-2-(methylthio)tetrahydro-1H- |
| | pyrrolizin-7a(5H)-yl)methoxy)-4-(1,4-oxazepan-4-yl)pyrido[4,3-d] |
| | pyrimidin-7-yl)-5-(methylthio)naphthalen-2-ol; |
| 421 | 4-(4-(2-oxa-6-azabicyclo[5.1.0]octan-6-yl)-8-fluoro-2-(((2R,7aS)-2- |
| | fluorotetrahydro-1H-pyrrolizin-7a(5H)-yl)methoxy)pyrido[4,3-d] |
| | pyrimidin-7-yl)-6-fluoro-5-(methylthio)naphthalen-2-ol; |
| 422 | 4-(4-(2-oxa-6-azabicyclo[5.1.0]octan-6-yl)-8-fluoro-2-(((2R,7aS)-2- |
| | (methylthio)tetrahydro-1H-pyrrolizin-7a(5H)-yl)methoxy)pyrido |
| | [4,3-d]pyrimidin-7-yl)-6-fluoro-5-(methylthio)naphthalen-2-ol; |
| 423 | 4-(4-(2-oxa-6-azabicyclo[5.1.0]octan-6-yl-8,8-d2)-8-fluoro-2- |
| | (((2R,7aS)-2-methoxytetrahydro-1H-pyrrolizin-7a(5H)-yl)methoxy) |
| | pyrido[4,3-d]pyrimidin-7-yl)-5-ethyl-6-fluoronaphthalen-2-ol; |
| 424 | 4-(4-(2-oxa-6-azabicyclo[5.1.0]octan-6-yl-8,8-d2)-8-fluoro-2- |
| | (((2R,7aS)-2-methoxytetrahydro-1H-pyrrolizin-7a(5H)-yl)methoxy) |
| | pyrido[4,3-d]pyrimidin-7-yl)-5-ethynyl-6-fluoronaphthalen-2-ol; |
| 425 | 4-(4-(2-oxa-6-azabicyclo[5.1.0]octan-6-yl-8,8-d2)-8-fluoro-2- |
| | (((2R,7aS)-2-fluoro-2-methyltetrahydro-1H-pyrrolizin-7a(5H)-yl) |
| | methoxy)pyrido[4,3-d]pyrimidin-7-yl)-5-ethyl-6-fluoronaphthalen- |
| | 2-ol; |
| 426 | 4-(4-(2-oxa-6-azabicyclo[5.1.0]octan-6-yl-8,8-d2)-8-fluoro-2- |
| | (((2R,7aS)-2-fluoro-2-methyltetrahydro-1H-pyrrolizin-7a(5H)- |
| | yl)methoxy)pyrido[4,3-d]pyrimidin-7-yl)-5-ethynyl-6- |
| | fluoronaphthalen-2-ol; |
| 427 | 4-(4-((1S,7R)-2-oxa-6-azabicyclo[5.1.0]octan-6-yl)-8-fluoro-2- |
| | (((2R,7aS)-2-methoxytetrahydro-1H-pyrrolizin-7a(5H)-yl)methoxy) |
| | pyrido[4,3-d]pyrimidin-7-yl)-5-ethyl-6-fluoronaphthalen-2-ol; |
| 428 | 4-(4-((1S,7R)-2-oxa-6-azabicyclo[5.1.0]octan-6-yl)-8-fluoro-2- |
| | (((2R,7aS)-2-fluoro-2-methyltetrahydro-1H-pyrrolizin-7a(5H)-yl) |
| | methoxy)pyrido[4,3-d]pyrimidin-7-yl)-5-ethyl-6-fluoronaphthalen- |
| | 2-ol; |
| 429 | 4-(4-((1R,7S)-2-oxa-6-azabicyclo[5.1.0]octan-6-yl)-8-fluoro-2- |
| | (((2R,7aS)-2-methoxytetrahydro-1H-pyrrolizin-7a(5H)-yl)methoxy) |
| | pyrido[4,3-d]pyrimidin-7-yl)-5-ethyl-6-fluoronaphthalen-2-ol; or |
| 430 | 4-(4-((1R,7S)-2-oxa-6-azabicyclo[5.1.0]octan-6-yl)-8-fluoro-2- |
| | (((2R,7aS)-2-fluoro-2-methyltetrahydro-1H-pyrrolizin-7a(5H)-yl) |
| | methoxy)pyrido[4,3-d]pyrimidin-7-yl)-5-ethyl-6-fluoronaphthalen- |
| | 2-ol. |

[71]. A pharmaceutical composition, comprising a therapeutically effective amount of the compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], and a pharmaceutically acceptable excipient.

[72]. A method for treating cancer in a subject comprising administering a therapeutically effective amount of the compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] to a subject in need thereof.

[73]. A method for treating cancer in a subject in need thereof, the method comprising:
(a) determining whether the cancer is associated with K-Ras G12C, K-Ras G12D), K-Ras G12V, K-Ras G13D, K-Ras G12R, K-Ras G12S, K-Ras G12A, K-Ras Q61H mutation and/or K-Ras wild type amplification; and
(b) if so, administering a therapeutically effective amount of the compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] to the subject in need thereof.

[74]. The compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] for use in therapy.

[75]. The compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] for use as a medicament.

[76]. The compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] for use in a method for the treatment of cancer.

[77]. A use of the compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] for the treatment of cancer.

[78]. A use of the compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of any one of [1] to [70], or the pharmaceutical composition of [71] for the manufacture of a medicament for the treatment of cancer.

[79]. The method for treating cancer of [72], the use in a method for the treatment of cancer of [76], the use for the treatment of cancer of [77], or the use for the manufacture of a medicament for the treatment of cancer of [78], wherein, said cancer is selected from pancreatic carcinoma, colorectal carcinoma, lung carcinoma (such as non-small cell lung cancer), breast carcinoma, large intestine carcinoma, stomach carcinoma, endometrial carcinoma, esophageal carcinoma or gastroesophageal junction carcinoma.

[80]. The method for treating cancer of [72] or [79], the use in a method for the treatment of cancer of [76] or [79], the use for the treatment of cancer of [77] or [79], or the use for the manufacture of a medicament for the treatment of cancer of [78] or [79], wherein, the cancer is associated with at least one of K-Ras G12C, K-Ras G12D, K-Ras G12V, K-Ras G13D, K-Ras G12R, K-Ras G12S, K-Ras G12A, K-Ras Q61H mutation and/or K-Ras wild type amplification.

[81]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12C associated cancer.

[82]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12D associated cancer.

[83]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12V associated cancer.

[84]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G13D associated cancer.

[85]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12R associated cancer.

[86]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12S associated cancer.

[87]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12A associated cancer.

[88]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras Q61H associated cancer.

[89]. The method for treating cancer of [72], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras wild type cancer.

[90]. An intermediate selected from any compound in the Table 19:

TABLE 19

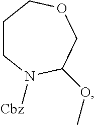 [SMILES: COC1COCCCN1C(=O)OCc1ccccc1]

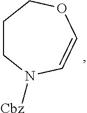 [SMILES: O=C(OCc1ccccc1)N1C=COCCC1]

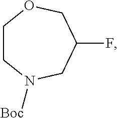 [SMILES: CC(C)(C)OC(=O)N1CCOCC(F)C1]

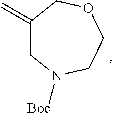

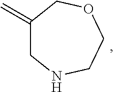

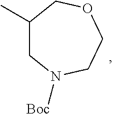

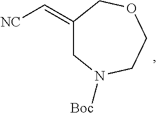 [SMILES: CC(C)(C)OC(=O)N1CCOC/C(=C/C#N)C1]

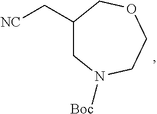 [SMILES: CC(C)(C)OC(=O)N1CCOCC(CC#N)C1]

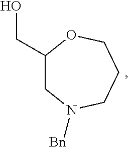 [SMILES: OCC1CN(Cc2ccccc2)CCCO1]

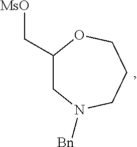 [SMILES: CS(=O)(=O)OCC1CN(Cc2ccccc2)CCCO1]

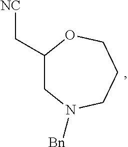 [SMILES: N#CCC1CN(Cc2ccccc2)CCCO1]

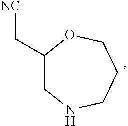

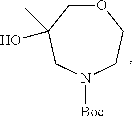 [SMILES: CC1(O)COCCN(C(=O)OC(C)(C)C)C1]

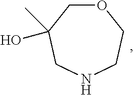

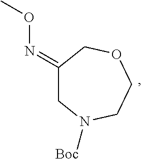 [SMILES: CO/N=C1\COCCN(C(=O)OC(C)(C)C)C1]

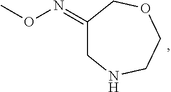

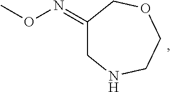

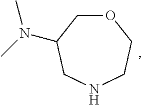

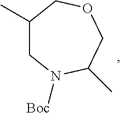

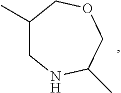

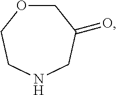

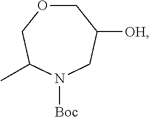 [SMILES: CC1COCC(O)CN1C(=O)OC(C)(C)C]

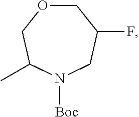 [SMILES: CC1COCC(F)CN1C(=O)OC(C)(C)C]

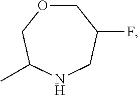

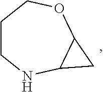

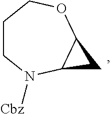 [SMILES: O=C(OCc1ccccc1)N1CCCO[C@H]2C[C@H]21]

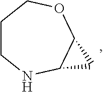

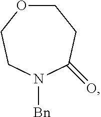 [SMILES: O=C1CCOCCN1Cc1ccccc1]

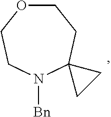 [SMILES: c1ccc(CN2CCOCCC23CC3)cc1]

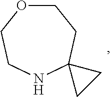

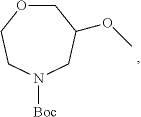 [SMILES: COC1COCCN(C(=O)OC(C)(C)C)C1]

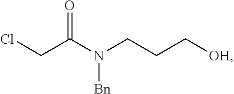 [SMILES: O=C(CCl)N(CCCO)Cc1ccccc1]

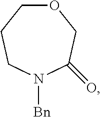 [SMILES: O=C1COCCCN1Cc1ccccc1]

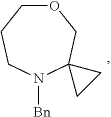 [SMILES: c1ccc(CN2CCCOCC23CC3)cc1]

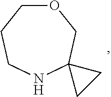

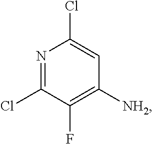

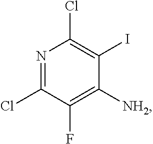 [SMILES: Nc1c(F)c(Cl)nc(Cl)c1I]

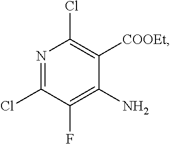 [SMILES: CCOC(=O)c1c(Cl)nc(Cl)c(F)c1N]

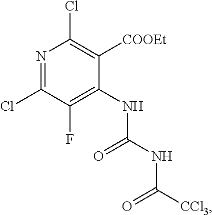

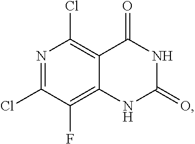 [SMILES: O=c1[nH]c(=O)c2c(Cl)nc(Cl)c(F)c2[nH]1]

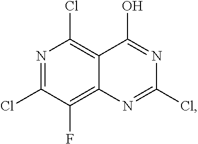 [SMILES: Oc1nc(Cl)nc2c(F)c(Cl)nc(Cl)c12]

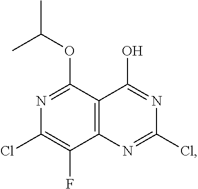 [SMILES: CC(C)Oc1nc(Cl)c(F)c2nc(Cl)nc(O)c12]

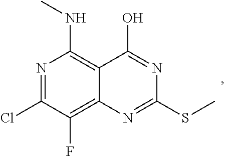 [SMILES: CNc1nc(Cl)c(F)c2nc(SC)nc(O)c12]

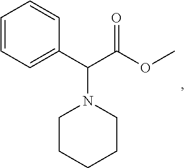 [SMILES: COC(=O)C(c1ccccc1)N1CCCCC1]

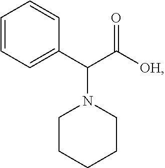 [SMILES: O=C(O)C(c1ccccc1)N1CCCCC1]

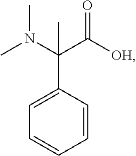

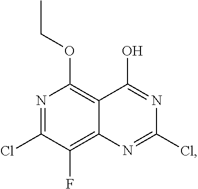 [SMILES: CCOc1nc(Cl)c(F)c2nc(Cl)nc(O)c12]

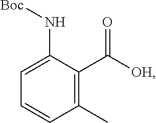 [SMILES: Cc1cccc(NC(=O)OC(C)(C)C)c1C(=O)O]

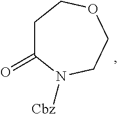 [SMILES: O=C1CCOCCN1C(=O)OCc1ccccc1]

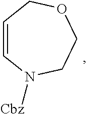 [SMILES: O=C(OCc1ccccc1)N1C=CCOCC1]

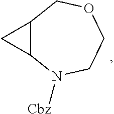 [SMILES: O=C(OCc1ccccc1)N1CCOCC2CC21]

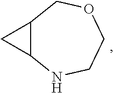

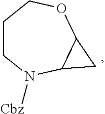 [SMILES: O=C(OCc1ccccc1)N1CCCOC2CC21]

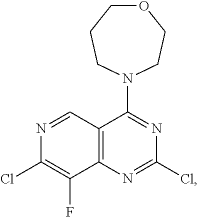 [SMILES: Fc1c(Cl)ncc2c(N3CCCOCC3)nc(Cl)nc12]

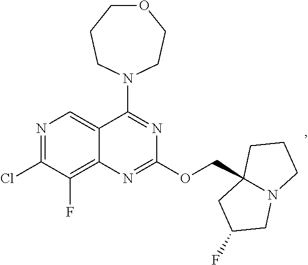 [SMILES: Fc1c(Cl)ncc2c(N3CCCOCC3)nc(OC[C@@]34CCCN3C[C@H](F)C4)nc12]

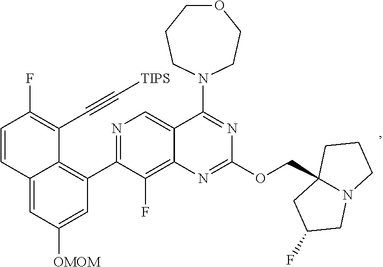

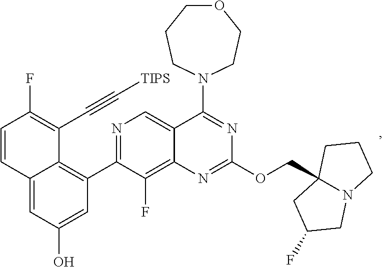

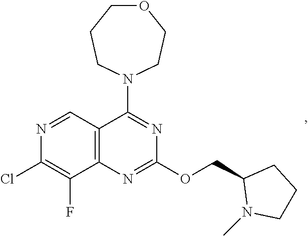 [SMILES: CN1CCC[C@@H]1COc1nc(N2CCCOCC2)c2cnc(Cl)c(F)c2n1]

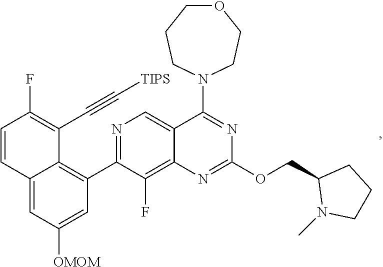

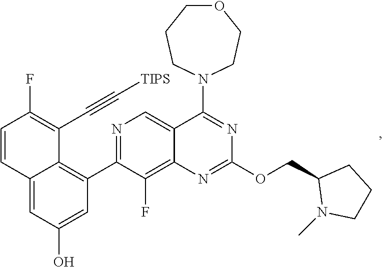

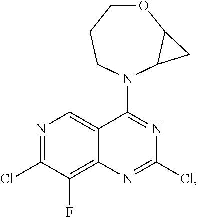 [SMILES: Fc1c(Cl)ncc2c(N3CCCOC4CC43)nc(Cl)nc12]

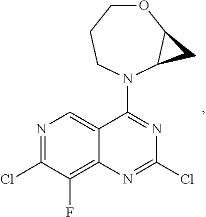 [SMILES: Fc1c(Cl)ncc2c(N3CCCO[C@H]4C[C@H]43)nc(Cl)nc12]

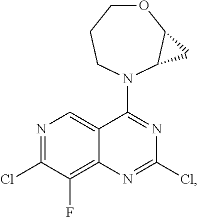 [SMILES: Fc1c(Cl)ncc2c(N3CCCO[C@@H]4C[C@@H]43)nc(Cl)nc12]

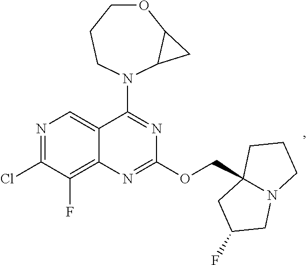 [SMILES: Fc1c(Cl)ncc2c(N3CCCOC4CC43)nc(OC[C@@]34CCCN3C[C@H](F)C4)nc12]

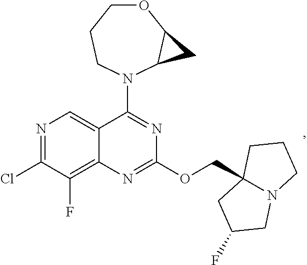 [SMILES: Fc1c(Cl)ncc2c(N3CCCO[C@H]4C[C@H]43)nc(OC[C@@]34CCCN3C[C@H](F)C4)nc12]

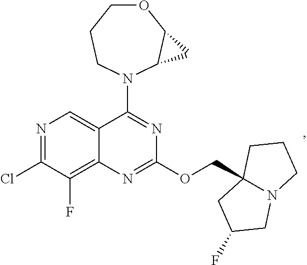 [SMILES: Fc1c(Cl)ncc2c(N3CCCO[C@@H]4C[C@@H]43)nc(OC[C@@]34CCCN3C[C@H](F)C4)nc12]

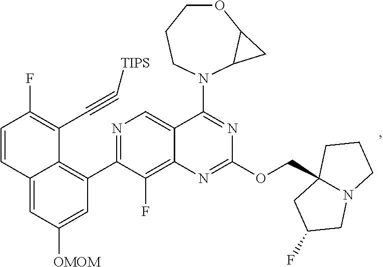

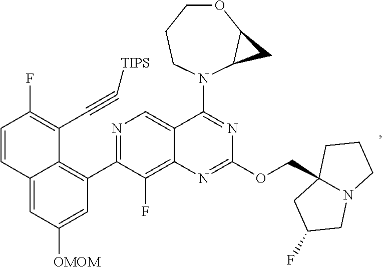

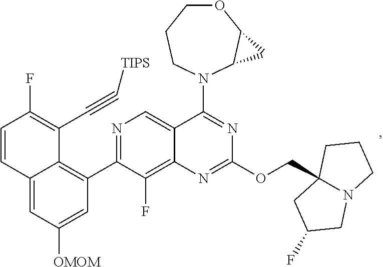

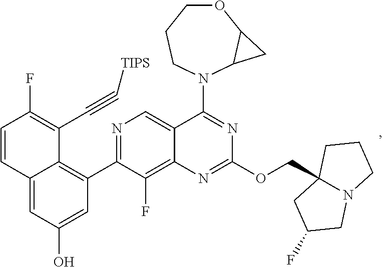

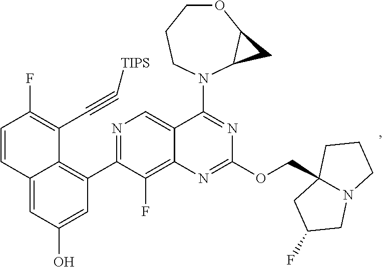

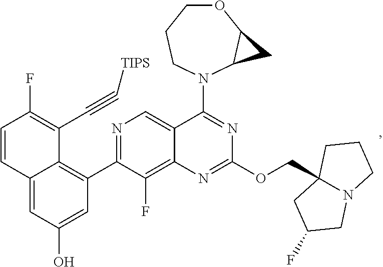

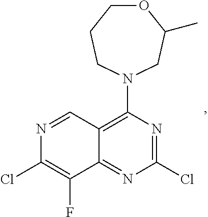 [SMILES: CC1CN(c2nc(Cl)nc3c(F)c(Cl)ncc23)CCCO1]

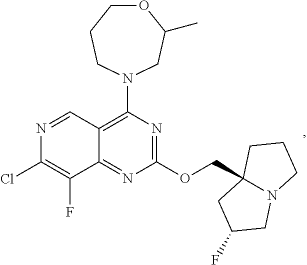 [SMILES: CC1CN(c2nc(OC[C@@]34CCCN3C[C@H](F)C4)nc3c(F)c(Cl)ncc23)CCCO1]

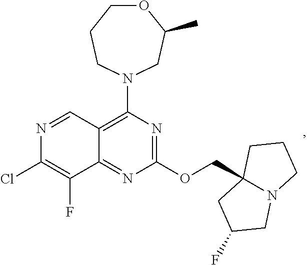 [SMILES: C[C@H]1CN(c2nc(OC[C@@]34CCCN3C[C@H](F)C4)nc3c(F)c(Cl)ncc23)CCCO1]

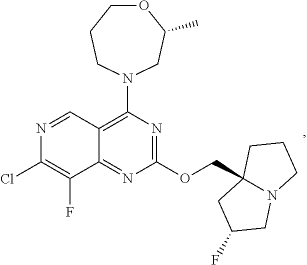 [SMILES: C[C@@H]1CN(c2nc(OC[C@@]34CCCN3C[C@H](F)C4)nc3c(F)c(Cl)ncc23)CCCO1]

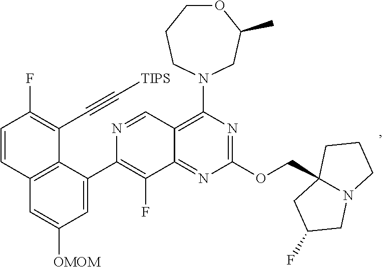

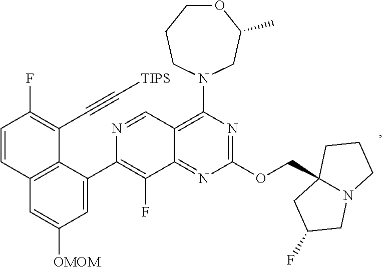

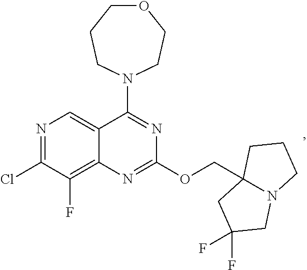 [SMILES: Fc1c(Cl)ncc2c(N3CCCOCC3)nc(OCC34CCCN3CC(F)(F)C4)nc12]

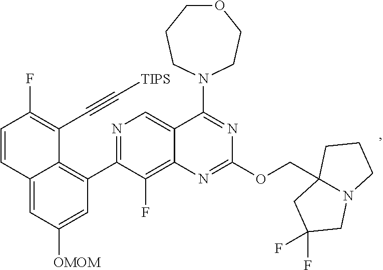

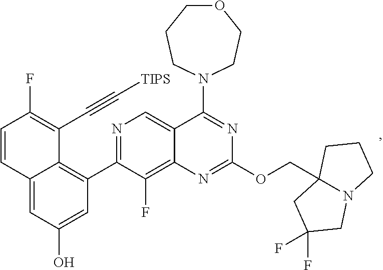

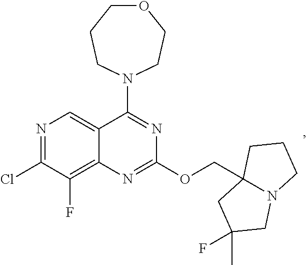 [SMILES: CC1(F)CN2CCCC2(COc2nc(N3CCCOCC3)c3cnc(Cl)c(F)c3n2)C1]

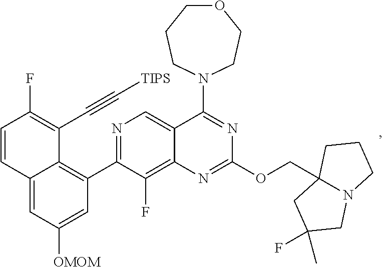

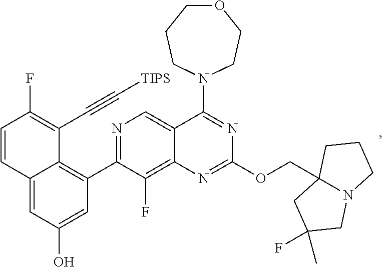

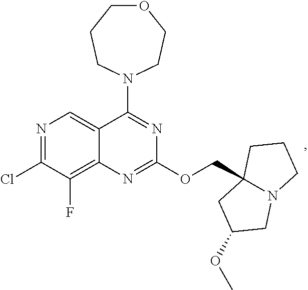 [SMILES: CO[C@H]1CN2CCC[C@@]2(COc2nc(N3CCCOCC3)c3cnc(Cl)c(F)c3n2)C1]

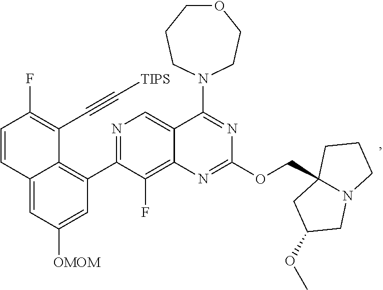

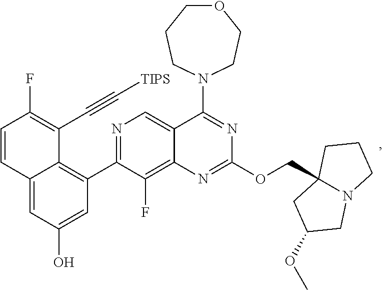

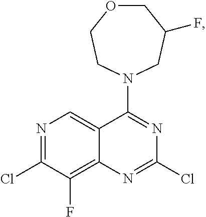 [SMILES: Fc1c(Cl)ncc2c(N3CCOCC(F)C3)nc(Cl)nc12]

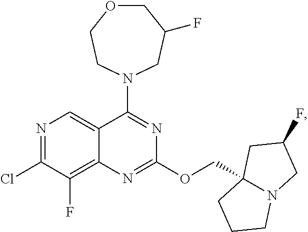 [SMILES: Fc1c(Cl)ncc2c(N3CCOCC(F)C3)nc(OC[C@@]34CCCN3C[C@H](F)C4)nc12]

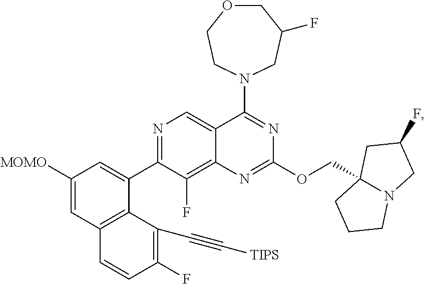

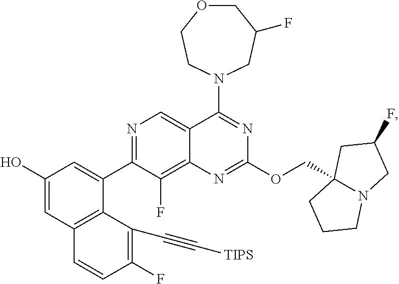

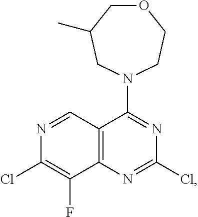 [SMILES: CC1COCCN(c2nc(Cl)nc3c(F)c(Cl)ncc23)C1]

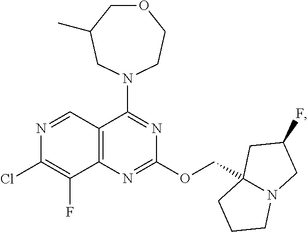

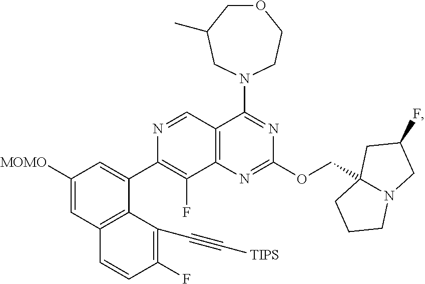

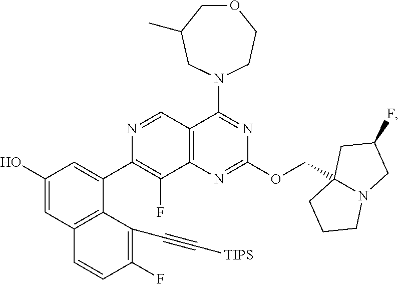

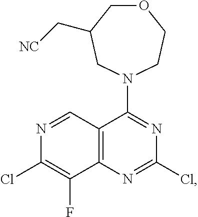 [SMILES: N#CCC1COCCN(c2nc(Cl)nc3c(F)c(Cl)ncc23)C1]

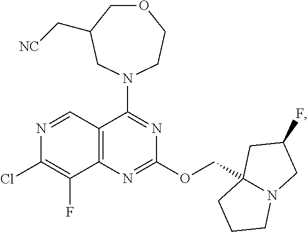

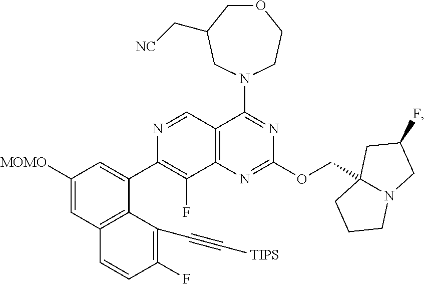

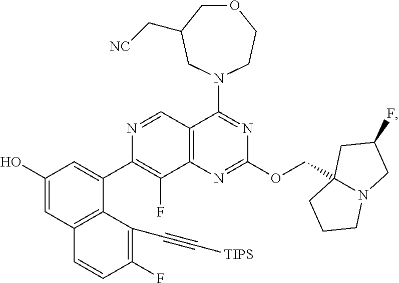

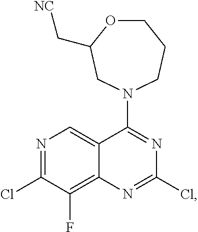 [SMILES: N#CCC1CN(c2nc(Cl)nc3c(F)c(Cl)ncc23)CCCO1]

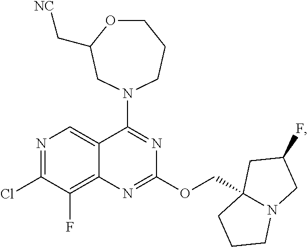 [SMILES: N#CCC1CN(c2nc(OC[C@@]34CCCN3C[C@H](F)C4)nc3c(F)c(Cl)ncc23)CCCO1]

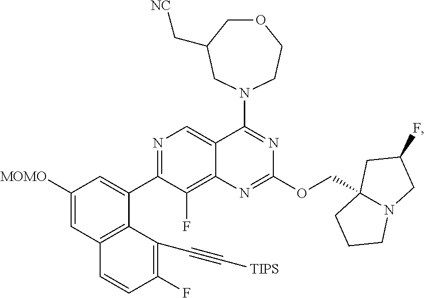

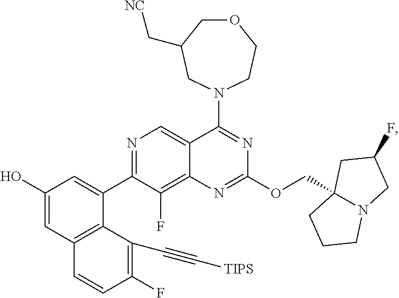

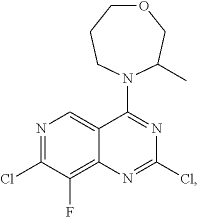 [SMILES: CC1COCCCN1c1nc(Cl)nc2c(F)c(Cl)ncc12]

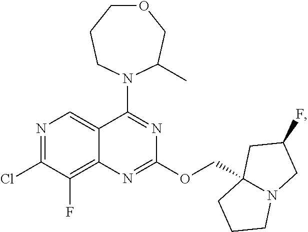 [SMILES: CC1COCCCN1c1nc(OC[C@@]23CCCN2C[C@H](F)C3)nc2c(F)c(Cl)ncc12]

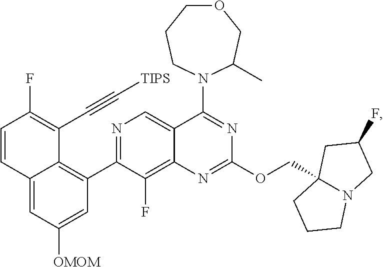

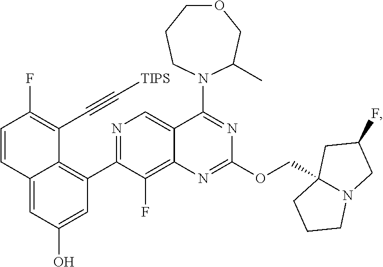

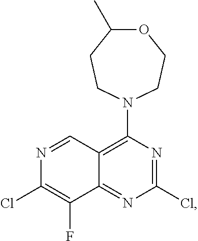 [SMILES: CC1CCN(c2nc(Cl)nc3c(F)c(Cl)ncc23)CCO1]

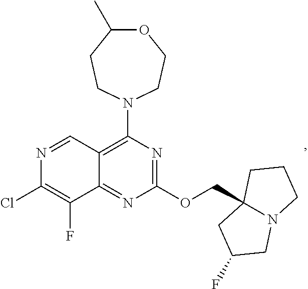 [SMILES: CC1CCN(c2nc(OC[C@@]34CCCN3C[C@H](F)C4)nc3c(F)c(Cl)ncc23)CCO1]

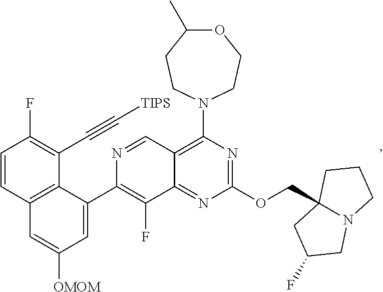

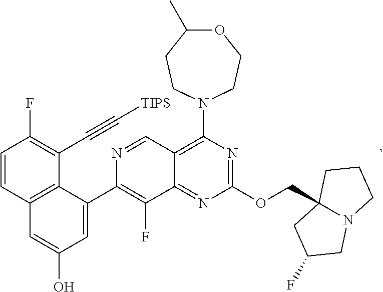

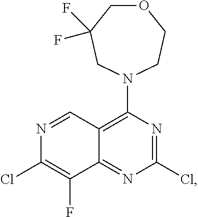 [SMILES: Fc1c(Cl)ncc2c(N3CCOCC(F)(F)C3)nc(Cl)nc12]

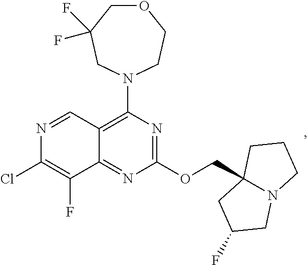 [SMILES: Fc1c(Cl)ncc2c(N3CCOCC(F)(F)C3)nc(OC[C@@]34CCCN3C[C@H](F)C4)nc12]

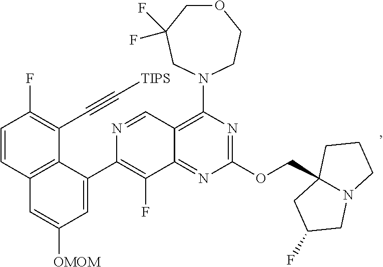

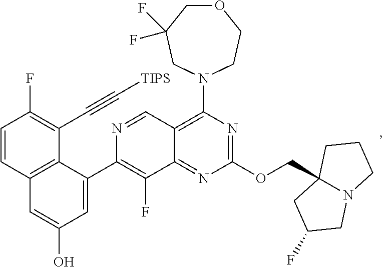

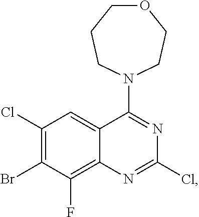 [SMILES: Fc1c(Br)c(Cl)cc2c(N3CCCOCC3)nc(Cl)nc12]

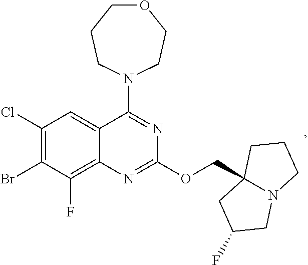 [SMILES: Fc1c(Br)c(Cl)cc2c(N3CCCOCC3)nc(OC[C@@]34CCCN3C[C@H](F)C4)nc12]

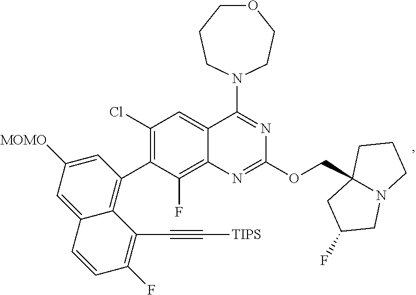

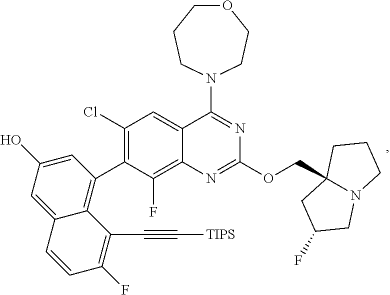

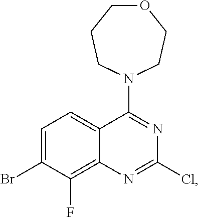 [SMILES: Fc1c(Br)ccc2c(N3CCCOCC3)nc(Cl)nc12]

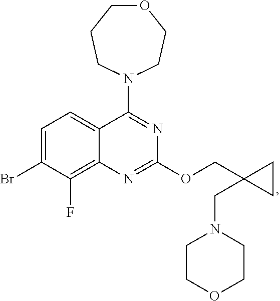 [SMILES: Fc1c(Br)ccc2c(N3CCCOCC3)nc(OCC3(CN4CCOCC4)CC3)nc12]

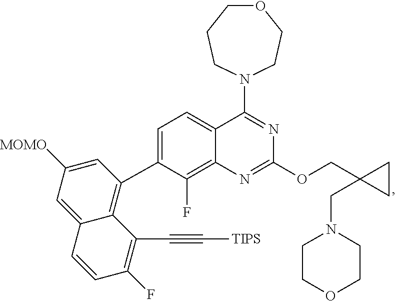

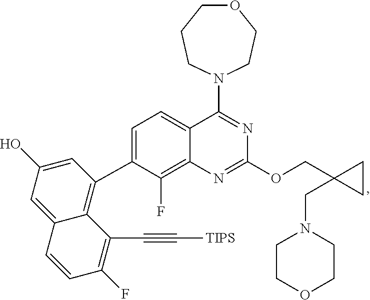

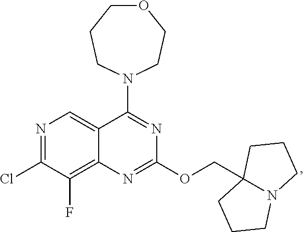 [SMILES: Fc1c(Cl)ncc2c(N3CCCOCC3)nc(OCC34CCCN3CCC4)nc12]

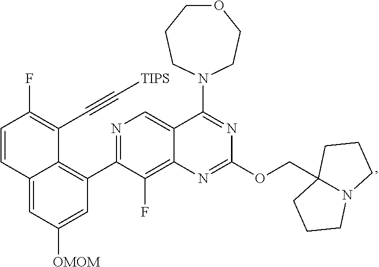

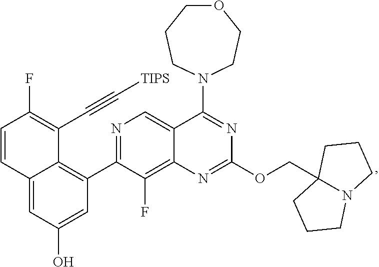

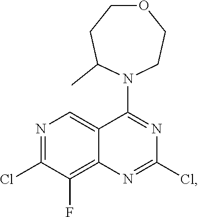 [SMILES: CC1CCOCCN1c1nc(Cl)nc2c(F)c(Cl)ncc12]

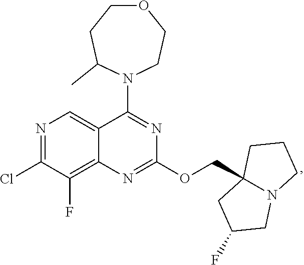 [SMILES: CC1CCOCCN1c1nc(OC[C@@]23CCCN2C[C@H](F)C3)nc2c(F)c(Cl)ncc12]

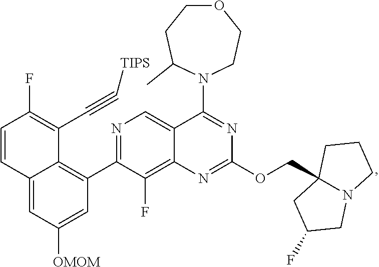

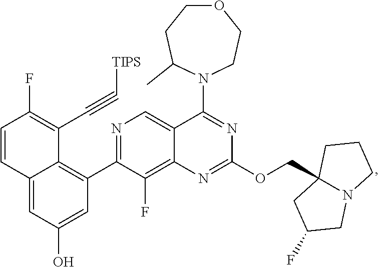

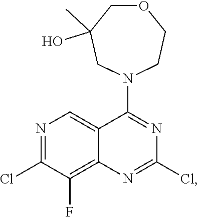 [SMILES: CC1(O)COCCN(c2nc(Cl)nc3c(F)c(Cl)ncc23)C1]

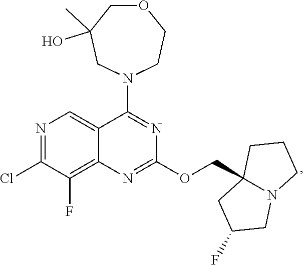 [SMILES: CC1(O)COCCN(c2nc(OC[C@@]34CCCN3C[C@H](F)C4)nc3c(F)c(Cl)ncc23)C1]

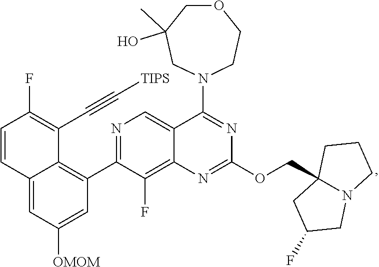

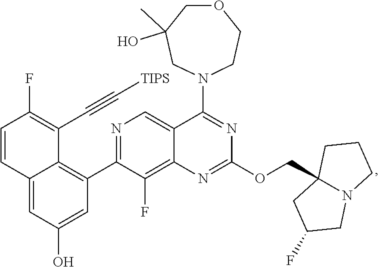

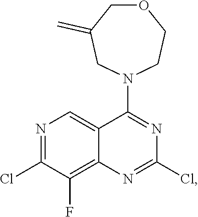 [SMILES: C=C1COCCN(c2nc(Cl)nc3c(F)c(Cl)ncc23)C1]

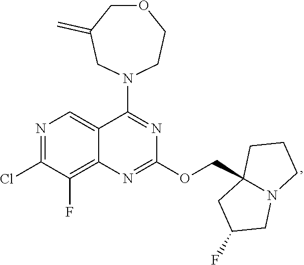 [SMILES: C=C1COCCN(c2nc(OC[C@@]34CCCN3C[C@H](F)C4)nc3c(F)c(Cl)ncc23)C1]

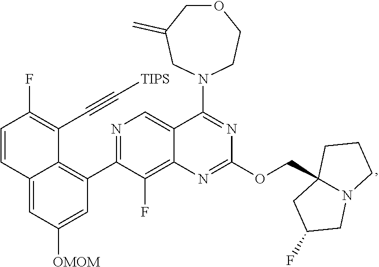

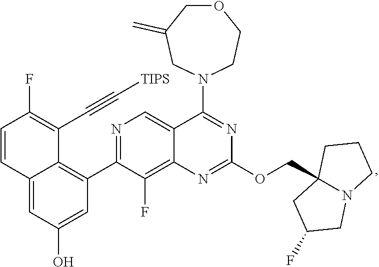

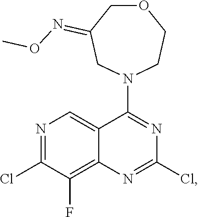 [SMILES: CO/N=C1/COCCN(c2nc(Cl)nc3c(F)c(Cl)ncc23)C1]

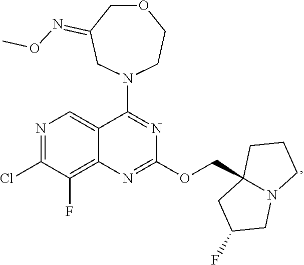 [SMILES: CO/N=C1/COCCN(c2nc(OC[C@@]34CCCN3C[C@H](F)C4)nc3c(F)c(Cl)ncc23)C1]

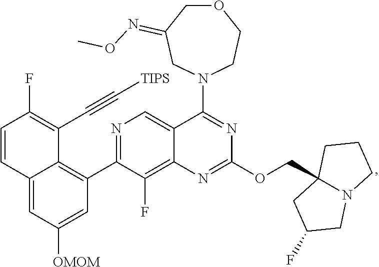

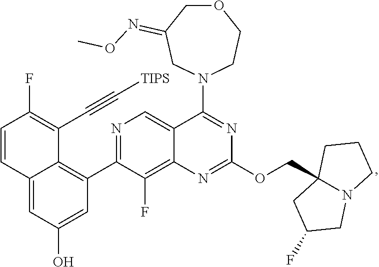

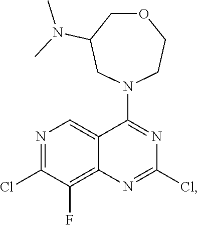 [SMILES: CN(C)C1COCCN(c2nc(Cl)nc3c(F)c(Cl)ncc23)C1]

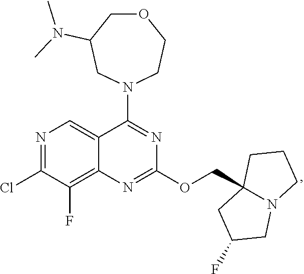 [SMILES: CN(C)C1COCCN(c2nc(OC[C@@]34CCCN3C[C@H](F)C4)nc3c(F)c(Cl)ncc23)C1]

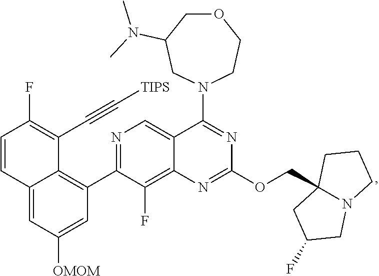

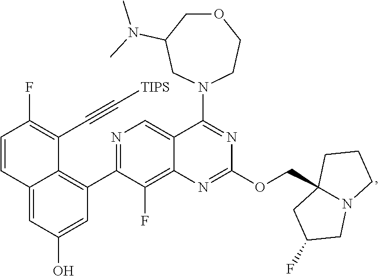

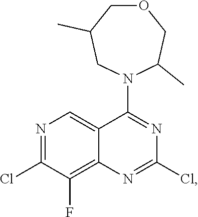 [SMILES: CC1COCC(C)N(c2nc(Cl)nc3c(F)c(Cl)ncc23)C1]

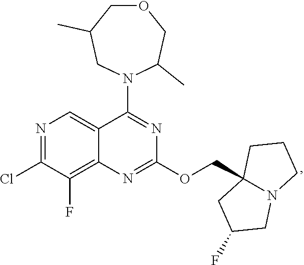 [SMILES: CC1COCC(C)N(c2nc(OC[C@@]34CCCN3C[C@H](F)C4)nc3c(F)c(Cl)ncc23)C1]

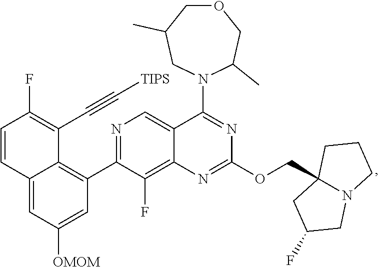

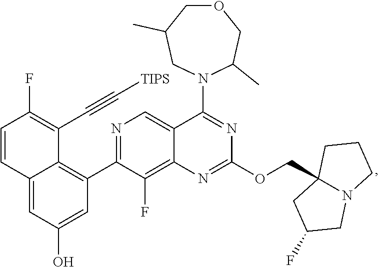

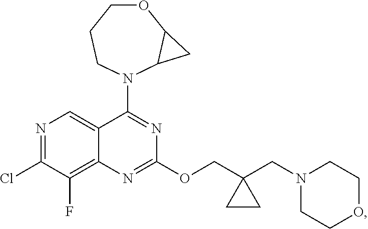 [SMILES: Fc1c(Cl)ncc2c(N3CCCOC4CC43)nc(OCC3(CN4CCOCC4)CC3)nc12]

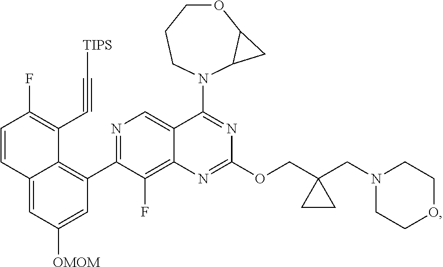

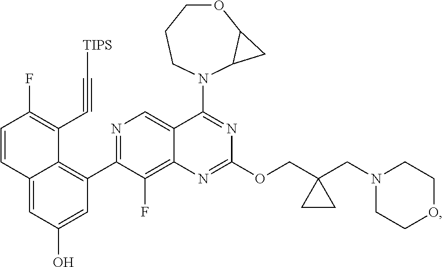

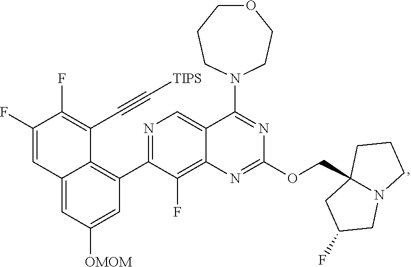

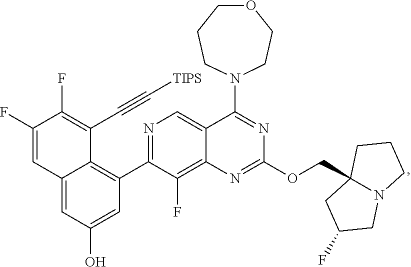

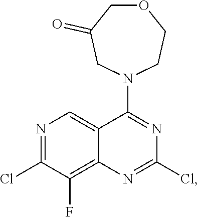 [SMILES: O=C1COCCN(c2nc(Cl)nc3c(F)c(Cl)ncc23)C1]

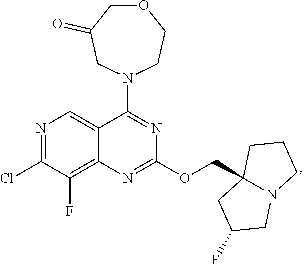 [SMILES: O=C1COCCN(c2nc(OC[C@@]34CCCN3C[C@H](F)C4)nc3c(F)c(Cl)ncc23)C1]

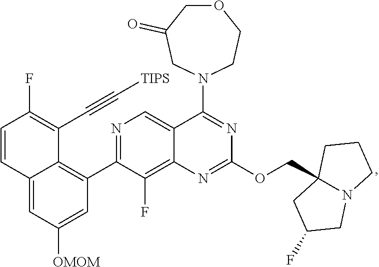

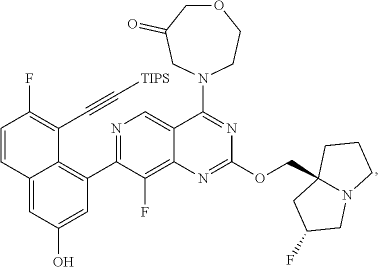

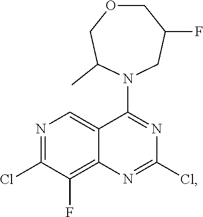 [SMILES: CC1COCC(F)CN1c1nc(Cl)nc2c(F)c(Cl)ncc12]

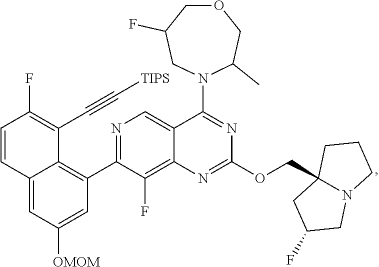

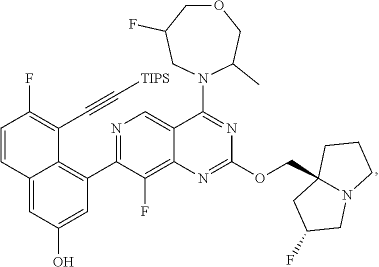

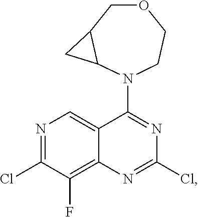 [SMILES: Fc1c(Cl)ncc2c(N3CCOCC4CC43)nc(Cl)nc12]

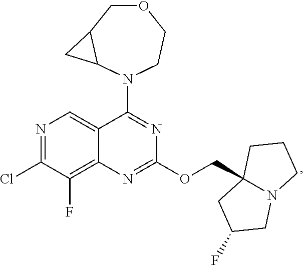 [SMILES: Fc1c(Cl)ncc2c(N3CCOCC4CC43)nc(OC[C@@]34CCCN3C[C@H](F)C4)nc12]

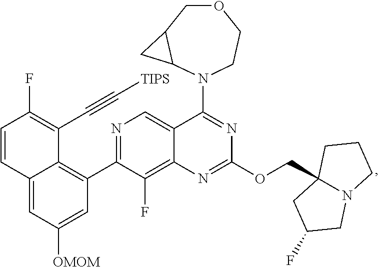

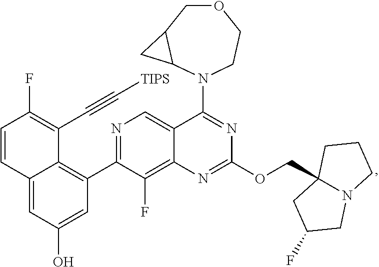

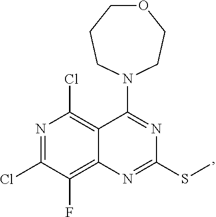

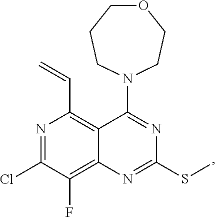

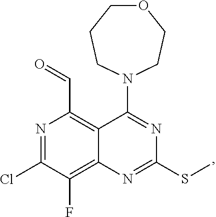

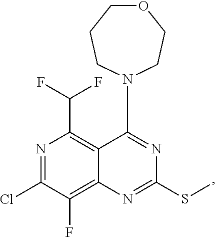

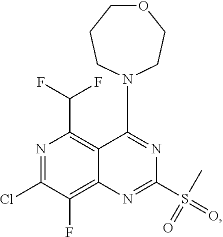 [SMILES: CS(=O)(=O)c1nc(N2CCCOCC2)c2c(C(F)F)nc(Cl)c(F)c2n1]

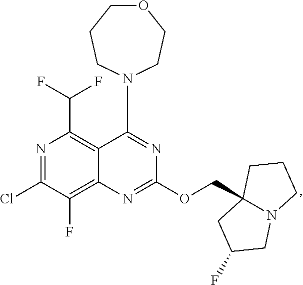 [SMILES: Fc1c(Cl)nc(C(F)F)c2c(N3CCCOCC3)nc(OC[C@@]34CCCN3C[C@H](F)C4)nc12]

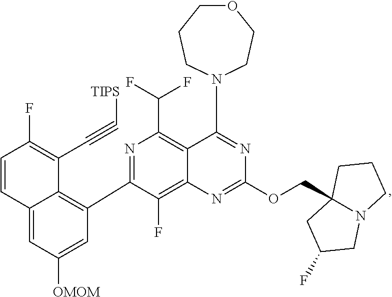

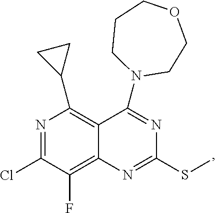

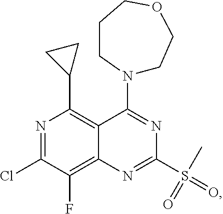 [SMILES: CS(=O)(=O)c1nc(N2CCCOCC2)c2c(C3CC3)nc(Cl)c(F)c2n1]

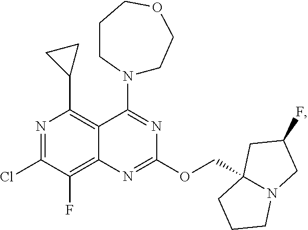 [SMILES: Fc1c(Cl)nc(C2CC2)c2c(N3CCCOCC3)nc(OC[C@@]34CCCN3C[C@H](F)C4)nc12]

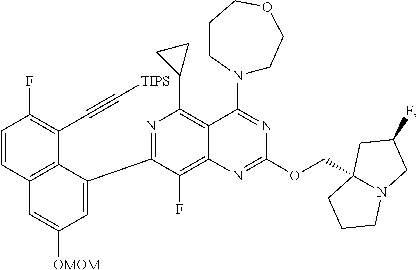

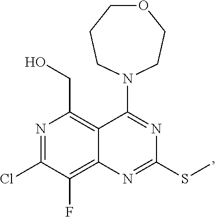

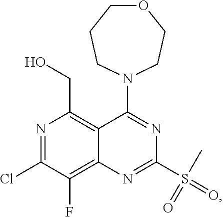 [SMILES: CS(=O)(=O)c1nc(N2CCCOCC2)c2c(CO)nc(Cl)c(F)c2n1]

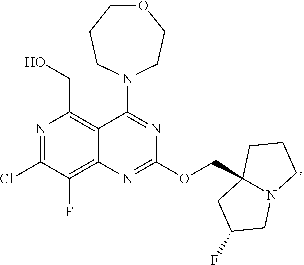 [SMILES: OCc1nc(Cl)c(F)c2nc(OC[C@@]34CCCN3C[C@H](F)C4)nc(N3CCCOCC3)c12]

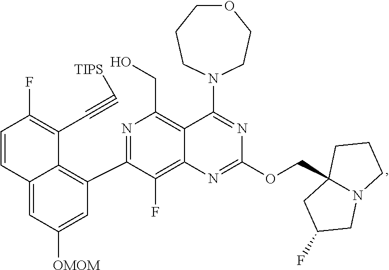

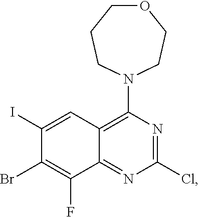 [SMILES: Fc1c(Br)c(I)cc2c(N3CCCOCC3)nc(Cl)nc12]

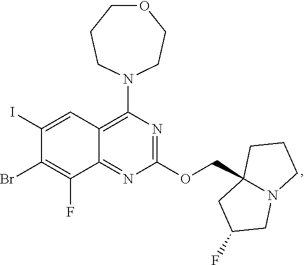 [SMILES: Fc1c(Br)c(I)cc2c(N3CCCOCC3)nc(OC[C@@]34CCCN3C[C@H](F)C4)nc12]

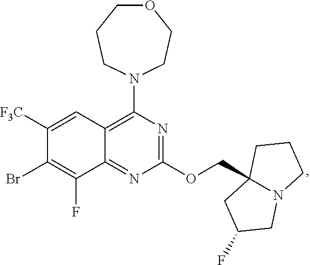 [SMILES: Fc1c(Br)c(C(F)(F)F)cc2c(N3CCCOCC3)nc(OC[C@@]34CCCN3C[C@H](F)C4)nc12]

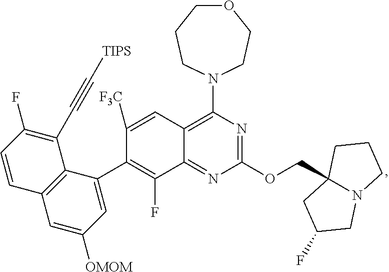

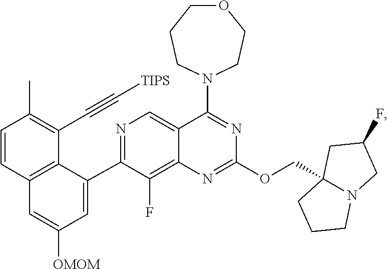

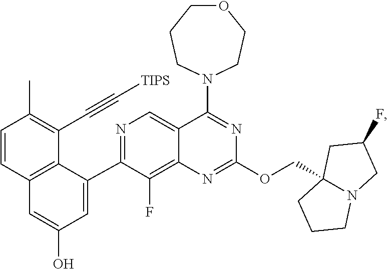

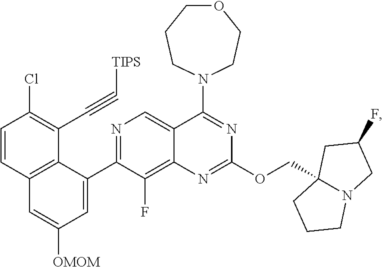

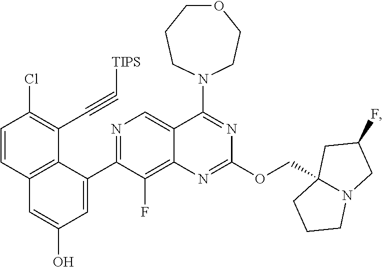

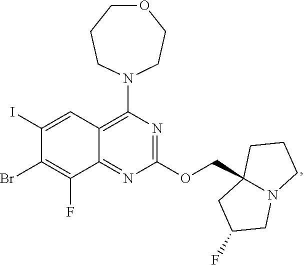 [SMILES: Fc1c(Br)c(I)cc2c(N3CCCOCC3)nc(OC[C@@]34CCCN3C[C@H](F)C4)nc12]

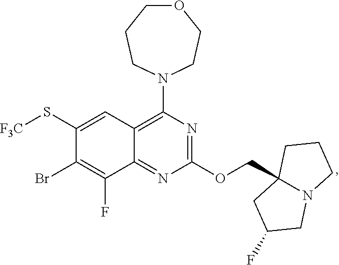 [SMILES: Fc1c(Br)c(SC(F)(F)F)cc2c(N3CCCOCC3)nc(OC[C@@]34CCCN3C[C@H](F)C4)nc12]

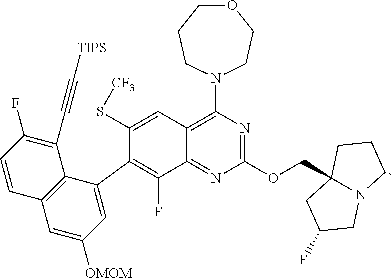

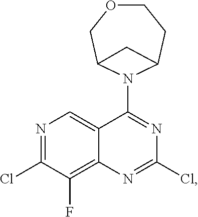 [SMILES: Fc1c(Cl)ncc2c(N3C4CCOCC3C4)nc(Cl)nc12]

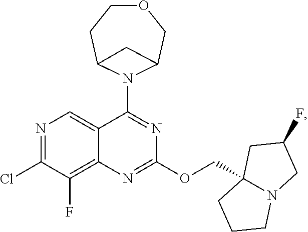 [SMILES: Fc1c(Cl)ncc2c(N3C4CCOCC3C4)nc(OC[C@@]34CCCN3C[C@H](F)C4)nc12]

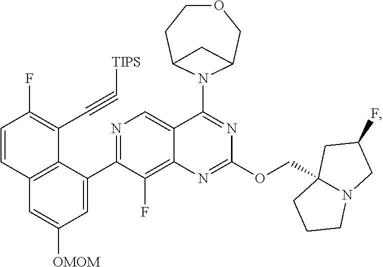

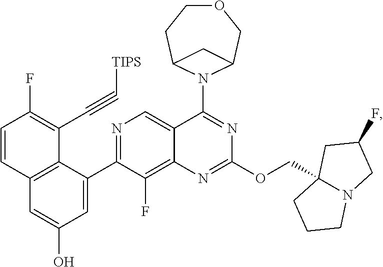

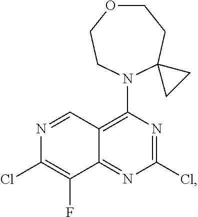 [SMILES: Fc1c(Cl)ncc2c(N3CCOCCC34CC4)nc(Cl)nc12]

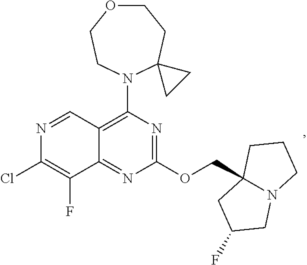 [SMILES: Fc1c(Cl)ncc2c(N3CCOCCC34CC4)nc(OC[C@@]34CCCN3C[C@H](F)C4)nc12]

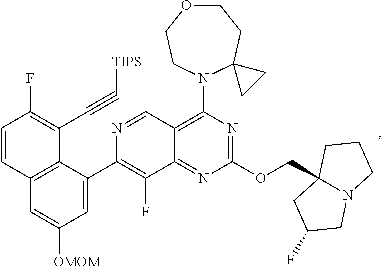

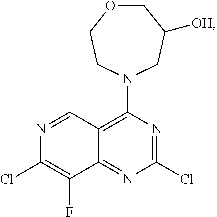 [SMILES: OC1COCCN(c2nc(Cl)nc3c(F)c(Cl)ncc23)C1]

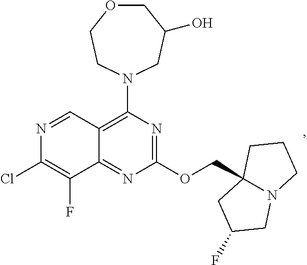 [SMILES: OC1COCCN(c2nc(OC[C@@]34CCCN3C[C@H](F)C4)nc3c(F)c(Cl)ncc23)C1]

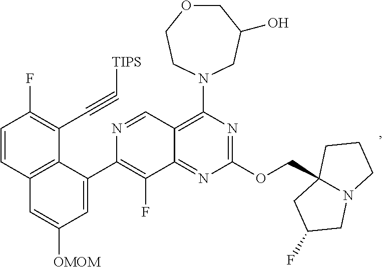

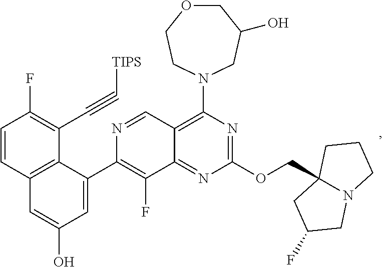

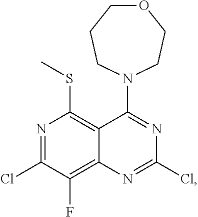 [SMILES: CSc1nc(Cl)c(F)c2nc(Cl)nc(N3CCCOCC3)c12]

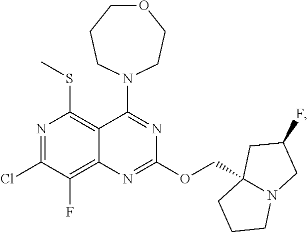 [SMILES: CSc1nc(Cl)c(F)c2nc(OC[C@@]34CCCN3C[C@H](F)C4)nc(N3CCCOCC3)c12]

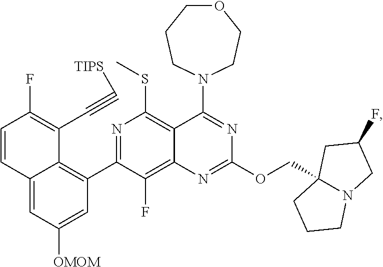

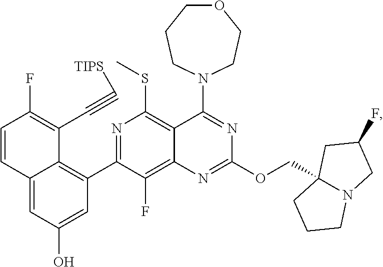

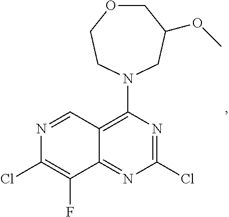 [SMILES: COC1COCCN(c2nc(Cl)nc3c(F)c(Cl)ncc23)C1]

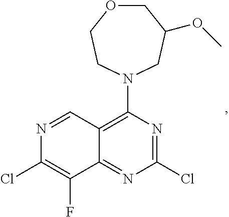 [SMILES: COC1COCCN(c2nc(Cl)nc3c(F)c(Cl)ncc23)C1]

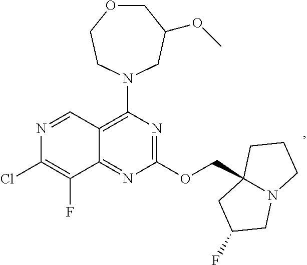 [SMILES: COC1COCCN(c2nc(OC[C@@]34CCCN3C[C@H](F)C4)nc3c(F)c(Cl)ncc23)C1]

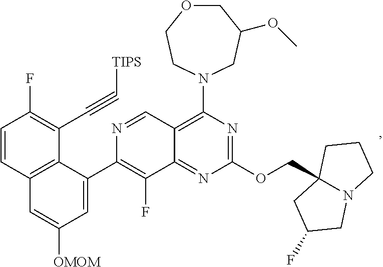

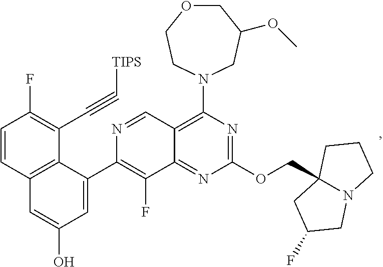

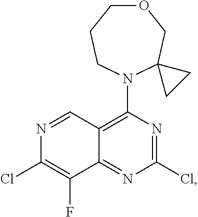 [SMILES: Fc1c(Cl)ncc2c(N3CCCOCC34CC4)nc(Cl)nc12]

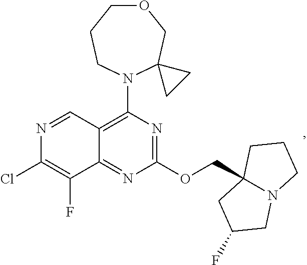 [SMILES: Fc1c(Cl)ncc2c(N3CCCOCC34CC4)nc(OC[C@@]34CCCN3C[C@H](F)C4)nc12]

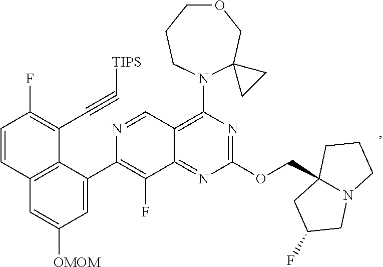

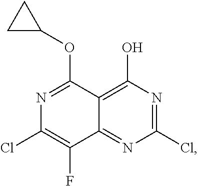 [SMILES: Oc1nc(Cl)nc2c(F)c(Cl)nc(OC3CC3)c12]

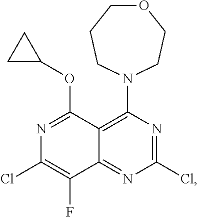 [SMILES: Fc1c(Cl)nc(OC2CC2)c2c(N3CCCOCC3)nc(Cl)nc12]

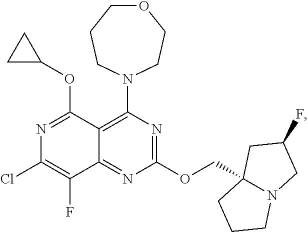 [SMILES: Fc1c(Cl)nc(OC2CC2)c2c(N3CCCOCC3)nc(OC[C@@]34CCCN3C[C@H](F)C4)nc12]

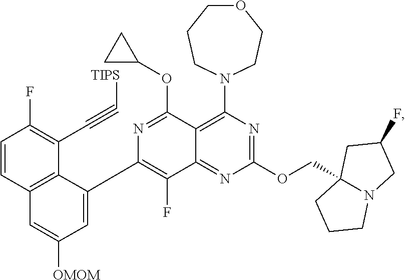

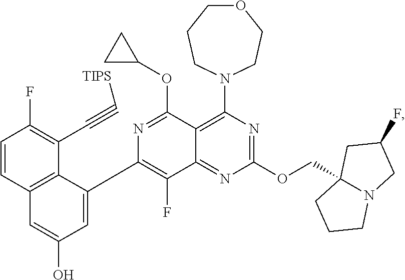

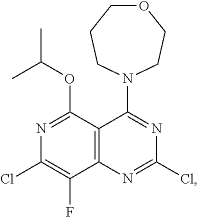 [SMILES: CC(C)Oc1nc(Cl)c(F)c2nc(Cl)nc(N3CCCOCC3)c12]

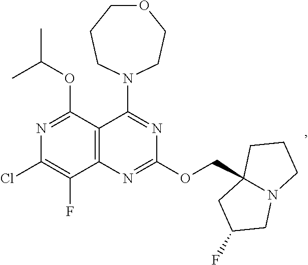 [SMILES: CC(C)Oc1nc(Cl)c(F)c2nc(OC[C@@]34CCCN3C[C@H](F)C4)nc(N3CCCOCC3)c12]

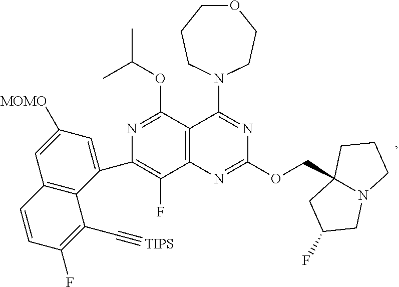

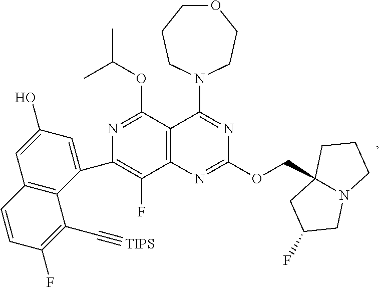

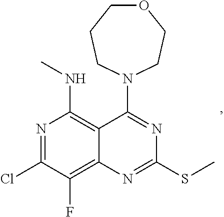 [SMILES: CNc1nc(Cl)c(F)c2nc(SC)nc(N3CCCOCC3)c12]

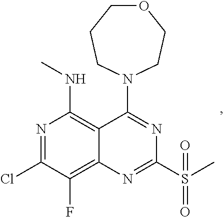 [SMILES: CNc1nc(Cl)c(F)c2nc(S(C)(=O)=O)nc(N3CCCOCC3)c12]

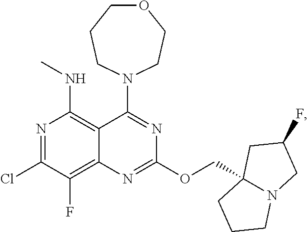 [SMILES: CNc1nc(Cl)c(F)c2nc(OC[C@@]34CCCN3C[C@H](F)C4)nc(N3CCCOCC3)c12]

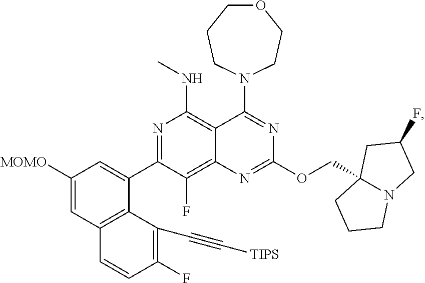

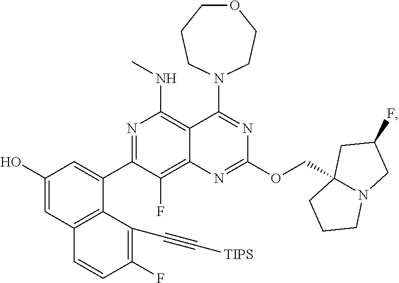

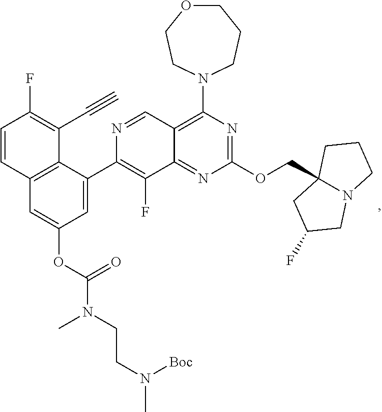 [SMILES: C#Cc1c(F)ccc2cc(OC(=O)N(C)CCN(C)C(=O)OC(C)(C)C)cc(-c3ncc4c(N5CCCOCC5)nc(OC[C@@]56CCCN5C[C@H](F)C6)nc4c3F)c12]

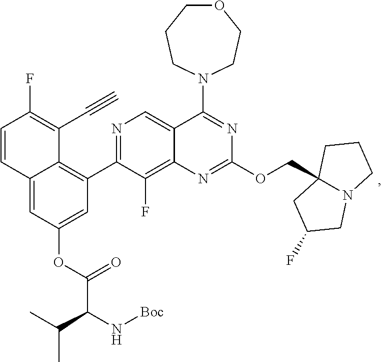 [SMILES: C#Cc1c(F)ccc2cc(OC(=O)[C@@H](NC(=O)OC(C)(C)C)C(C)C)cc(-c3ncc4c(N5CCCOCC5)nc(OC[C@@]56CCCN5C[C@H](F)C6)nc4c3F)c12]

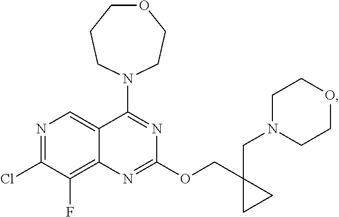 [SMILES: Fc1c(Cl)ncc2c(N3CCCOCC3)nc(OCC3(CN4CCOCC4)CC3)nc12]

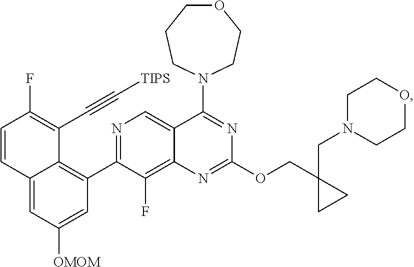

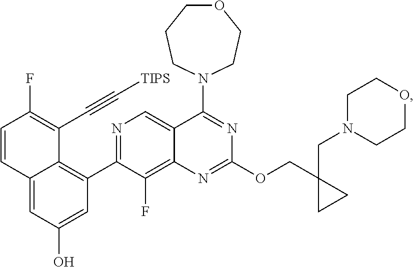

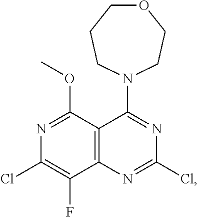 [SMILES: COc1nc(Cl)c(F)c2nc(Cl)nc(N3CCCOCC3)c12]

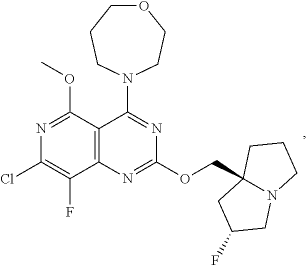 [SMILES: COc1nc(Cl)c(F)c2nc(OC[C@@]34CCCN3C[C@H](F)C4)nc(N3CCCOCC3)c12]

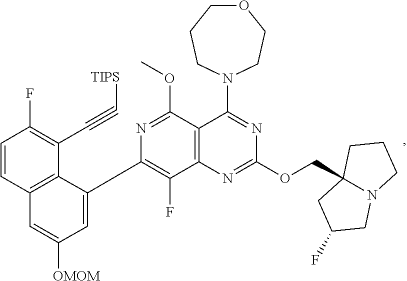

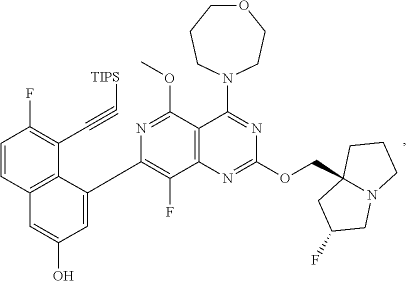

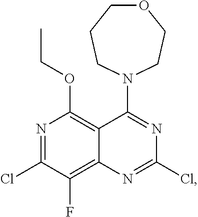 [SMILES: CCOc1nc(Cl)c(F)c2nc(Cl)nc(N3CCCOCC3)c12]

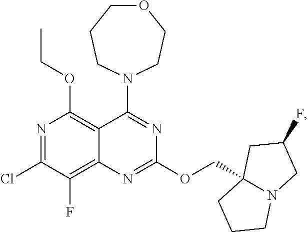 [SMILES: CCOc1nc(Cl)c(F)c2nc(OC[C@@]34CCCN3C[C@H](F)C4)nc(N3CCCOCC3)c12]

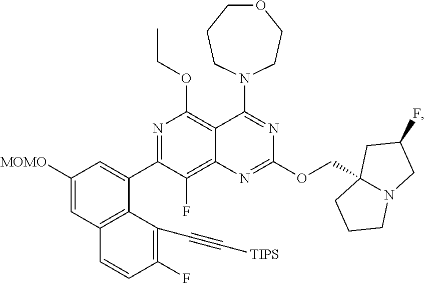

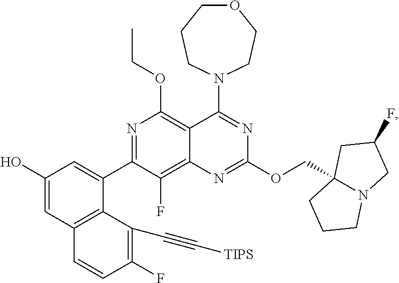

2745

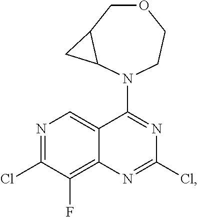 [SMILES: Fc1c(Cl)ncc2c(N3CCOCC4CC43)nc(Cl)nc12]

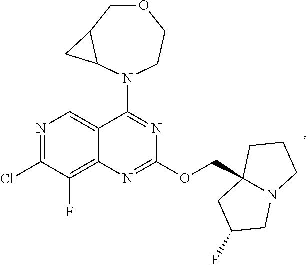 [SMILES: Fc1c(Cl)ncc2c(N3CCOCC4CC43)nc(OC[C@@]34CCCN3C[C@H](F)C4)nc12]

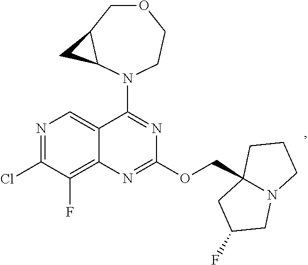 [SMILES: Fc1c(Cl)ncc2c(N3CCOC[C@@H]4C[C@@H]43)nc(OC[C@@]34CCCN3C[C@H](F)C4)nc12]

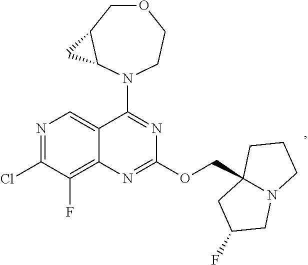 [SMILES: Fc1c(Cl)ncc2c(N3CCOC[C@H]4C[C@H]43)nc(OC[C@@]34CCCN3C[C@H](F)C4)nc12]

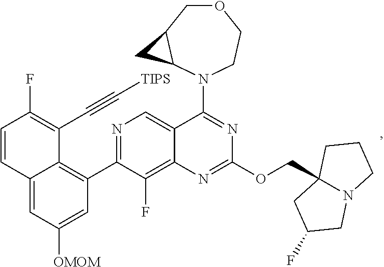

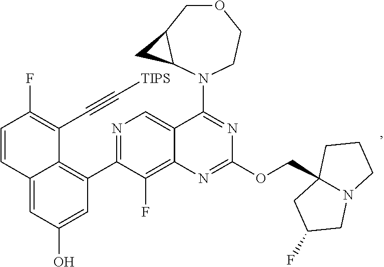

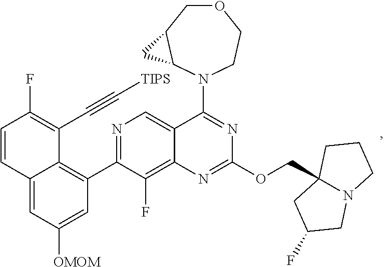

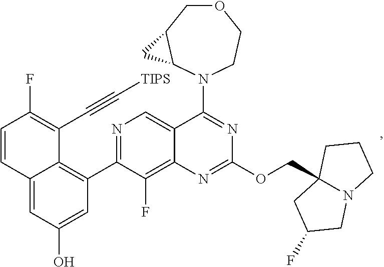

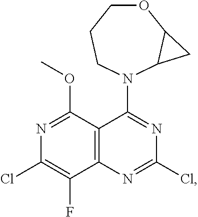 [SMILES: COc1nc(Cl)c(F)c2nc(Cl)nc(N3CCCOC4CC43)c12]

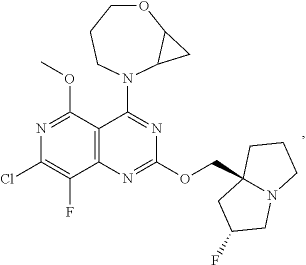 [SMILES: COc1nc(Cl)c(F)c2nc(OC[C@@]34CCCN3C[C@H](F)C4)nc(N3CCCOC4CC43)c12]

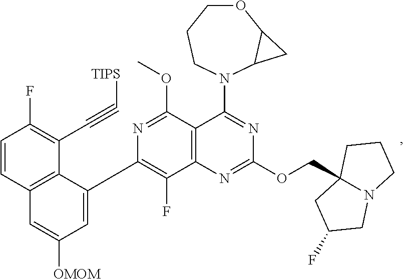

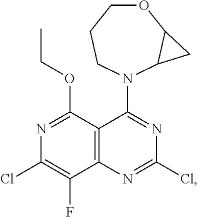 [SMILES: CCOc1nc(Cl)c(F)c2nc(Cl)nc(N3CCCOC4CC43)c12]

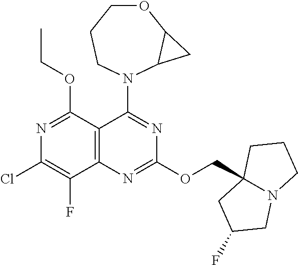 [SMILES: CCOc1nc(Cl)c(F)c2nc(OC[C@@]34CCCN3C[C@H](F)C4)nc(N3CCCOC4CC43)c12]

or

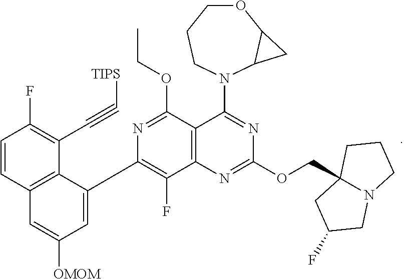

### Definitions

Unless defined otherwise, all technical and scientific terms used herein have the same meaning as is commonly understood by one of skill in the art to which this invention belongs. All patents, patent applications, and publications referred to herein are incorporated by reference.

The term “a”, “an”, “the” and similar terms, as used herein, unless otherwise indicated, are to be construed to cover both the singular and plural.

The term “halogen” or “halo”, as used interchangeably herein, unless otherwise indicated, refers to fluoro, chloro, bromo or iodo. The preferred halogen groups include —F, —Cl and —Br.

The term “alkyl”, as used herein, unless otherwise indicated, refers to saturated monovalent hydrocarbon radicals having straight or branched arrangement. C₁₋₁₀ in —C₁₋₁₀alkyl is defined to identify the group having 1, 2, 3, 4, 5, 6, 7, 8, 9 or 10 carbon atoms in a linear or branched arrangement. Non-limiting alkyl includes methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl.

The term “haloalkyl”, as used herein, unless otherwise indicated, refers to the above-mentioned alkyl substituted with one or more (for example 1, 2, 3, 4, 5, or 6) halogen (such as —F, —Cl or —Br). In some embodiments, the haloalkyl is interchangeable —C₁₋₁₀haloalkyl or haloC₁₋₁₀alkyl, wherein, C₁₋₁₀ in the —C₁₋₁₀haloalkyl or haloC₁₋₁₀alkyl indicates that the total carbon atoms of the alkyl are 1 to 10. In some embodiments, the —C₁₋₁₀haloalkyl is the —C₁₋₆haloalkyl. In some embodiments, the —C₁₋₆haloalkyl is the —C₁₋₃haloalkyl. In some embodiments, the —C₁₋₃haloalkyl is (methyl, ethyl, propyl or isopropyl) substituted with 1, 2, 3, 4, 5, or 6 —F; preferably, the —C₁₋₃haloalkyl is —CF₃.

The term “alkylene”, as used herein, unless otherwise indicated, refers to a divalent group obtained by removal of an additional hydrogen atom from an alkyl group defined above. In some embodiments, the alkylene is C₀₋₆alkylene. In some embodiments, the C₀₋₆alkylene is C₀₋₃alkylene. The C₀₋₆ in the front of the alkylene indicates the total carbon atoms in the alkylene are 0 to 6 and C₀ indicates the two ends of the alkylene are connected directly. Non-limiting alkylene includes methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl”, as used herein, unless otherwise indicated, refers to a straight or branch-chained hydrocarbon radical containing one or more double bonds and typically from 2 to 20 carbon atoms in length. In some embodiments, the alkenyl is —C₂₋₁₀alkenyl. In some embodiments, the —C₂₋₁₀alkenyl is —C₂₋₆alkenyl which contains from 2 to 6 carbon atoms. Non-limiting alkenyl includes ethenyl, propenyl, butenyl, 2-methyl-2-buten-1-yl, heptenyl, octenyl and the like.

The term “haloalkenyl”, as used herein, unless otherwise indicated, refers to the above-mentioned alkenyl substituted with one or more (for example 1, 2, 3, 4, 5, or 6) halogen (such as —F, —Cl or —Br). In some embodiments, the haloalkenyl is interchangeable —C₂₋₁₀haloalkenyl or haloC₂₋₁₀alkenyl, wherein, C₂₋₁₀ in the —C₂₋₁₀haloalkenyl or haloC₂₋₁₀alkenyl indicates that the total carbon atoms of the alkenyl are 2 to 10. In some embodiments, the —C₂₋₁₀haloalkenyl is the —C₂₋₆haloalkenyl. In some embodiments, the —C₂₋₆haloalkenyl is the —C₂₋₃haloalkenyl. In some embodiments, the —C₂₋₃haloalkenyl is (ethenyl or propenyl) substituted with 1, 2, 3, 4, 5, or 6 —F.

The term “alkynyl”, as used herein, unless otherwise indicated, refers to a straight or branch-chained hydrocarbon radical containing one or more triple bonds and typically from 2 to 20 carbon atoms in length. In some embodiments, the alkynyl is —C₂₋₁₀alkynyl. In some embodiments, the —C₂₋₁₀alkynyl is —C₂₋₆alkynyl which contains from 2 to 6 carbon atoms. Non-limiting alkynyl includes ethynyl, 1-propynyl, 1-butynyl, heptynyl, octynyl and the like.

The term “haloalkynyl”, as used herein, unless otherwise indicated, refers to the above-mentioned alkynyl substituted with one or more (for example 1, 2, 3, 4, 5, or 6) halogen (such as —F, —Cl or —Br). In some embodiments, the haloalkynyl is interchangeable —C₂₋₁₀haloalkynyl or haloC₂₋₁₀alkynyl, wherein, C₂₋₁₀ in the —C₂₋₁₀haloalkynyl or haloC₂₋₁₀alkynyl indicates that the total carbon atoms of the alkynyl are 2 to 10. In some embodiments, the —C₂₋₁₀haloalkynyl is the —C₂₋₆haloalkynyl. In some embodiments, the —C₂₋₆haloalkynyl is the —C₂₋₃haloalkynyl. In some embodiments, the —C₂₋₃haloalkynyl is (ethynyl or propynyl) substituted with 1, 2, 3, 4, 5, or 6 —F.

The term “alkoxy”, as used herein, unless otherwise indicated, refers to oxygen ethers formed from the previously described alkyl groups.

The term “haloalkoxy”, as used herein, unless otherwise indicated, refers to the above-mentioned alkoxy substituted with one or more (for 1, 2, 3, 4, 5, or 6) halogen (—F, —Cl or —Br). In some embodiment, the haloalkoxy is interchangeable —C₁₋₁₀haloalkoxy or haloC₁₋₁₀alkoxy. In some embodiments, the haloalkoxy is interchangeable —C₁₋₆haloalkoxy or haloC₁₋₆alkoxy, wherein, C₁₋₆ in the —C₁₋₆haloalkoxy or haloC₁₋₆alkoxy indicates that the total carbon atoms of the alkoxy are 1 to 6. In some embodiments, the —C₁₋₆haloalkoxy is the —C₁₋₃haloalkoxy. In some embodiments, the —C₁₋₃haloalkoxy is (methoxy, ethoxy, propoxy or isopropoxy) substituted with 1, 2, 3, 4, 5, or 6 —F; preferably, the —C₁₋₃haloalkoxy is —OCF₃.

The term “carbocyclic ring”, as used herein, unless otherwise indicated, refers to a totally saturated or partially saturated monocyclic, bicyclic, bridged, fused, or spiro non-aromatic ring only containing carbon atoms as ring members. The term “carbocyclyl” as used herein, unless otherwise indicated, means a monovalent group obtained by removal of a hydrogen atom on the ring carbon atom from the carbocyclic ring defined in the present invention. The carbocyclic ring is interchangeable with the carbocyclyl ring in the present invention. In some embodiments, the carbocyclic ring is a three to twenty membered (such as 3-, 4-, 5-, 6-, 7-, 8-, 9-, 10-, 11-, 12-, 13-, 14-, 15-, 16-, 17-, 18-, 19- or 20-membered) carbocyclic ring and is either fully saturated or has one or more degrees of unsaturation. Multiple degrees of substitution, for example, one, two, three, four, five or six, are included within the present definition. The carbocyclic ring includes a cycloalkyl ring in which all ring carbon atoms are saturated, a cycloalkenyl ring which contains at least one double bond (preferred contain one double bond), and a cycloalkynyl ring which contains at least one triple bond (preferred contain one triple bond). Cycloalkyl includes but not limited to cyclopropyl, cyclobutyl, cyclopentyl, cyclohexyl, cycloheptyl, cyclooctyl, cyclononyl, cyclodecyl and the like. Cycloalkenyl includes but not limited to cyclopentenyl, cyclohexenyl, cycloheptenyl, cyclooctenyl, cyclononenyl, cyclodecenyl and the like. The carbocyclyl ring includes a monocyclic carbocyclyl ring, and a bicyclic or polycyclic carbocyclyl ring in which one, two or three or more atoms are shared between the rings. The term “spirocyclic carbocyclic ring” refers to a carbocyclic ring in which each of the rings only shares one ring atom with the other ring. In some embodiments, the spirocyclic ring is bicyclic spirocyclic ring. The spirocyclic carbocyclic ring includes a spirocyclic cycloalkyl ring and a spirocyclic cycloalkenyl ring and a spirocyclic cycloalkynyl ring. The term “fused carbocyclic ring” refers to a carbocyclic ring in which each of the rings shares two adjacent ring atoms with the other ring. In some embodiments, the fused ring is a bicyclic fused ring. The fused carbocyclic ring includes a fused cycloalkyl ring and a fused cycloalkenyl ring and a fused cycloalkynyl ring. A monocyclic carbocyclic ring fused with an aromatic ring (such as phenyl) is included in the definition of the fused carbocyclic ring. The term “bridged carbocyclic ring” refers to a carbocyclic ring that includes at least two bridgehead carbon ring atoms and at least one bridging carbon atom. In some embodiments, the bridged ring is bicyclic bridged ring. The bridged carbocyclic ring includes a bicyclic bridged carbocyclic ring which includes two bridgehead carbon atoms and a polycyclic bridged carbocyclic ring which includes more than two bridgehead carbon atoms. The bridged carbocyclic ring includes a bridged cycloalkyl ring, a bridged cycloalkenyl ring and a bridged cycloalkynyl ring. Examples of monocyclic carbocyclyl and bicyclic carbocyclyl include but not limit to cyclopropyl, cyclobutyl, cyclopentyl, 1-cyclopent-1-enyl, 1-cyclopent-2-cnyl, 1-cyclopent-3-onyl, cyclohoxyl, 1-cyclohex-1-onyl, 1-cyclohex-2-cnyl, and 1-cyclohex-3-enyl.

The term “heterocyclic ring”, as used herein, unless otherwise indicated, refers to a totally saturated or partially saturated monocyclic, bicyclic, bridged, fused, or spiro non-aromatic ring containing not only carbon atoms as ring members and but also containing one or more (such as 1, 2, 3, 4, 5, or 6) heteroatoms as ring 55 members. Preferred heteroatoms include N, O, S, N-oxides, sulfur oxides, and sulfur dioxides. The term “heterocyclyl” as used herein, unless otherwise indicated, means a monovalent group obtained by removal of a hydrogen atom on the ring carbon atom or the ring heteroatom from the heterocyclic ring defined in the present invention. The heterocyclic ring is interchangeable with the heterocyclyl ring in the present invention. In some embodiments, the heterocyclic ring is a three to twenty membered (such as 3-, 4-, 5-, 6-, 7-, 8-, 9-, 10-, 11-, 12-, 13-, 14-, 15-, 16-, 17-, 18-, 19- or 20-membered) heterocyclic ring and is either fully saturated or has one or more degrees of unsaturation. Multiple degrees of substitution, for example, one, two, three, four, five or six, are included within the present definition. The heterocyclic ring includes a heterocycloalkyl ring in which all ring carbon atoms are saturated, a heterocycloalkenyl ring which contains at least one double bond (preferred contain one double bond), and a heterocycloalkynyl ring which contains at least one triple bond (preferred contain one triple bond). The heterocyclyl ring includes a monocyclic heterocyclyl ring, and a bicyclic or polycyclic heterocyclyl ring in which one, two or three or more atoms are shared between the rings. The term “spirocyclic heterocyclic ring” refers to a heterocyclic ring in which each of the rings only shares one ring atom with the other ring. In some embodiments, the spirocyclic ring is bicyclic spirocyclic ring. The spirocyclic heterocyclic ring includes a spirocyclic heterocycloalkyl ring and a spirocyclic heterocycloalkenyl ring and a spirocyclic heterocycloalkynyl ring. The term “fused heterocyclic ring” refers to a heterocyclic ring in which each of the rings shares two adjacent ring atoms with the other ring. In some embodiments, the fused ring is a bicyclic fused ring. The fused heterocyclic ring includes a fused heterocycloalkyl ring and a fused heterocycloalkenyl ring and a fused heterocycloalkynyl ring. A monocyclic heterocyclic ring fused with an aromatic ring (such as phenyl) is included in the definition of the fused heterocyclic ring. The term “bridged heterocyclic ring” refers to a heterocyclic ring that includes at least two bridgehead ring atoms and at least one bridging atom. In some embodiments, the bridged ring is bicyclic bridged ring. The bridged heterocyclic ring includes a bicyclic bridged heterocyclic ring which includes two bridgehead atoms and a polycyclic bridged heterocyclic ring which includes more than two bridgehead atoms. The bridged heterocyclic ring includes a bridged heterocycloalkyl ring, a bridged heterocycloalkenyl ring and a bridged heterocycloalkynyl ring. Examples of such heterocyclyl include but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “aryl”, as used herein, unless otherwise indicated, refers to a mono or polycyclic aromatic ring system only containing carbon ring atoms. The preferred aryls are monocyclic or bicyclic 6-10 membered aromatic rings. Phenyl and naphthyl are preferred aryls.

The term “heteroaryl”, as used herein, unless otherwise indicated, refers to an aromatic ring containing carbons and one or more (such as 1, 2, 3 or 4) heteroatoms selected from N, O or S. The heteroaryl may be monocyclic or polycyclic. A monocyclic heteroaryl group may have 1 to 4 heteroatoms in the ring, while a polycyclic heteroaryl may contain 1 to 10 heteroatoms. A polycyclic heteroaryl ring may contain fused ring junction, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms), preferred monocyclic heteroaryl is 5 membered heteroaryl including 1, 2, 3 or 4 heteroatoms selected from N, O or S, or 6 membered heteroaryl including 1 or 2 heteroatoms selected from N. Examples of heteroaryl groups include, but not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “one or more”, as used herein, unless otherwise indicated, refers to one or more than one. In some embodiments, “one or more” refers to 1, 2, 3, 4, 5 or 6. In some embodiments, “one or more” refers to 1, 2, 3 or 4. In some embodiments, “one or more” refers to 1, 2, or 3. In some embodiments, “one or more” refers to 1 or 2. In some embodiments, “one or more” refers to 1. In some embodiments, “one or more” refers to 2. In some embodiments, “one or more” refers to 3. In some embodiments, “one or more” refers to 4. In some embodiments, “one or more” refers to 5. In some embodiments, “one or more” refers to 6.

The term “substituted”, as used herein, unless otherwise indicated, refers to a hydrogen atom on the carbon atom or a hydrogen atom on the nitrogen atom is replaced by a substituent. When one or more substituents are substituted on a ring in the present invention, it means that each of substituents may be respectively independently substituted on every ring atom of the ring including but not limited to a ring carbon atom or a ring nitrogen atom. In addition, when the ring is a polycyclic ring, such as a fused ring, a bridged ring or a sprio ring, each of substituents may be respectively independently substituted on every ring atom of the polycyclic ring.

The term “oxo” refers to oxygen atom together with the attached carbon atom forms the group.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The term “pharmaceutically acceptable salt” refers to a salt prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrug of the compounds of this invention. In general, such prodrug will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers. The term “stereoisomer” as used in the present invention refers to an isomer in which atoms or groups of atoms in the molecule are connected to each other in the same order but differ in spatial arrangement, including conformational isomers and configuration isomers. The configuration isomers include geometric isomers and optical isomers, and optical isomers mainly include enantiomers and diastereomers. The invention includes all possible stereoisomers of the compound.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

The term “deuterated derivative”, used herein, unless otherwise indicated, refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivative described herein. Thus, unless otherwise stated, when a reference is made to a “deuterated derivative” of a compound of the disclosure, at least one hydrogen is replaced with deuterium at well above its natural isotopic abundance (which is typically about 0.015%) In some embodiments, the deuterated derivative of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at lease 6333.3 (95% deuterium incorporation, at least 6466.7 (97% deuterium incorporation, or at least 6600 (99% deuterium incorporation).

When a tautomer of the compound in the present invention exists, the present invention includes any possible tautomer and pharmaceutically acceptable salts thereof, and mixtures thereof, except where specifically stated otherwise.

The “conjugated form” refers to herein that the compound described herein is conjugated to another agent through a linker or not through a linker, wherein, the compound functions as a binder or a inhibitor of K-Ras protein (including K-Ras G12C, K-Ras G12D, K-Ras G12V, K-Ras G13D, K-Ras G12R, K-Ras G12S, K-Ras G12A, K-Ras Q61H mutant protein and K-Ras wild type protein) For example, the conjugated form is a PROTAC molecule, e.g. the compound is incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. Preferably, the portion of the PROTAC that engages the E3 ligase is connected to the portion of the PROTAC that engages the target protein via a linker which consists of a variable chain of atoms. Recruitment of K-Ras protein to the E3 ligase will thus result in the destruction of the K-Ras protein. The variable chain of atoms can include, for example, rings, heteroatoms, and/or repeating polymeric units. It can be rigid or flexible. It can be attached to the two portions described above using standard techniques in the art of organic synthesis.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, a compound of formula (I) or (II), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof as defined herein can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as oil-in-water emulsion or as a water-in-oil liquid emulsion. In addition to the common dosage forms set out above, the compound in the present invention or a pharmaceutically acceptable salt thereof may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds of the present invention or pharmaceutically acceptable salts thereof can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound in the present invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described herein or pharmaceutically acceptable salts thereof, may also be prepared in powder or liquid concentrate form.

Unless otherwise apparent from the context, when a value is expressed as “about” X or “approximately” X, the stated value of X will be understood to be accurate to +10%, preferably, +5%, +2%.

The term “subject” refers to an animal. In some embodiments, the animal is a mammal. A subject also refers to for example, primates (e.g., humans), cows, sheep, goats, horses, dogs, cats, rabbits, rats, mice, fish, birds and the like. In certain embodiments, the subject is a human. A “patient” as used herein refers to a human subject. As used herein, a subject is “in need of” a treatment if such subject would benefit biologically, medically or in quality of life from such treatment. In some embodiments, the subject has experienced and/or exhibited at least one symptom of cancer to be treated and/or prevented. In some embodiments, the subject has been identified or diagnosed as having a cancer having wild type K-Ras or a K-Ras G12A, K-Ras G12C, K-Ras G12D, K-Ras G12R, K-Ras G12S, K-Ras G12V, K-Ras G13D and/or K-Ras Q61H mutation

The term “inhibition”, “inhibiting” or “inhibit” refers to the reduction or suppression of a given condition, symptom, or disorder, or disease, or a significant decrease in the baseline activity of a biological activity or process.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, “K-Ras G12A” refers to a mutant form of a mammalian K-Ras protein that contains an amino acid substitution of an alanine for a glycine at amino acid position 12. A “K-Ras G12A inhibitor” refers to a compound is capable of negatively modulating or inhibiting all or a portion of the function of K-Ras G12A. A “K-Ras G12A associated cancer” as used herein refers to a cancer associated with or mediated by or having a K-Ras G12A mutation.

As used herein, “K-Ras G12C” refers to a mutant form of a mammalian K-Ras protein that contains an amino acid substitution of a cysteine for a glycine at amino acid position 12. A “K-Ras G12C inhibitor” refers to a compound is capable of negatively modulating or inhibiting all or a portion of the function of K-Ras G12C. A “K-Ras G12C associated cancer” as used herein refers to a cancer associated with or mediated by or having a K-Ras G12C mutation.

As used herein, “K-Ras G12D” refers to a mutant form of a mammalian K-Ras protein that contains an amino acid substitution of an aspartic acid for a glycine at amino acid position 12. A “K-Ras G12D inhibitor” refers to a compound is capable of negatively modulating or inhibiting all or a portion of the function of K-Ras G12D. A “K-Ras G12D associated cancer” as used herein refers to a cancer associated with or mediated by or having a K-Ras G12D mutation.

As used herein, “K-Ras G12R” refers to a mutant form of a mammalian K-Ras protein that contains an amino acid substitution of an arginine for a glycine at amino acid position 12. A “K-Ras G12R inhibitor” refers to a compound is capable of negatively modulating or inhibiting all or a portion of the function of K-Ras G12R. A “K-Ras G12R associated cancer” as used herein refers to a cancer associated with or mediated by or having a K-Ras G12R mutation.

As used herein, “K-Ras G12S” refers to a mutant form of a mammalian K-Ras protein that contains an amino acid substitution of a serine for a glycine at amino acid position 12. A “K-Ras G12S inhibitor” refers to a compound is capable of negatively modulating or inhibiting all or a portion of the function of K-Ras G12S. A “K-Ras G12S associated cancer” as used herein refers to a cancer associated with or mediated by or having a K-Ras G12S mutation.

As used herein, “K-Ras G12V” refers to a mutant form of a mammalian K-Ras protein that contains an amino acid substitution of a valine for a glycine at amino acid position 12. A “K-Ras G12V inhibitor” refers to a compound is capable of negatively modulating or inhibiting all or a portion of the function of K-Ras G12V. A “K-Ras G12V associated cancer” as used herein refers to a cancer associated with or mediated by or having a K-Ras G12V mutation.

As used herein, “K-Ras G13D” refers to a mutant form of a mammalian K-Ras protein that contains an amino acid substitution of an aspartic acid for a glycine at amino acid position 13. A “K-Ras G13D inhibitor” refers to a compound is capable of negatively modulating or inhibiting all or a portion of the function of K-Ras G13D. A “K-Ras G13D associated cancer” as used herein refers to a cancer associated with or mediated by or having a K-Ras G13D mutation.

As used herein, “K-Ras Q61H” refers to a mutant form of a mammalian K-Ras protein that contains an amino acid substitution of a histidine for a glutamine at amino acid position 61. A “K-Ras Q61H inhibitor” refers to a compound is capable of negatively modulating or inhibiting all or a portion of the function of K-Ras Q61H. A “K-Ras Q61H associated cancer” as used herein refers to a cancer associated with or mediated by or having a K-Ras Q61H mutation.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

These and other aspects will become apparent from the following written description of the invention.

### Methods of Preparation

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The following examples are provided to better illustrate the present invention. All parts and percentages are by weight and all temperatures are degrees Celsius, unless explicitly stated otherwise. The following abbreviations in the Table 20 have been used in the examples:

TABLE 20

| | |
|---|---|
| DMF | N,N-Dimethylformamide |
| EA/EtOAc | Ethyl acetate |
| Hex | Hexane |
| MeOH | Methanol |
| DCM | Dichloromethane |
| DCE | 1,2-Dichloroethane |
| EtOH | Ethanol |
| THF | Tetrahydrofuran |
| DIEA/DIPEA | N,N-Diisopropylethylamine |
| Pd(PPh₃)₄ | Tetrakis(triphenylphosphine)palladium |
| Pd(dppf)Cl₂ | [1,1′-Bis(diphenylphosphino)ferrocene]dichloropalladium(II) |
| TFA | Trifluoroacetic acid |
| ACN/MeCN/CH₃CN | Acetonitrile |
| Et₃N/TEA | Triethylamine |
| NIS | N-Iodosuccinimide |
| DMSO | Dimethyl sulfoxide |
| NCS | N-chlorosuccinimide |
| TBSCl | Tert-butyldimethylsilyl chloride\| |
| TMSCl | Trimethylsilyl chloride |
| MOMCl | Methoxymethyl chloride |
| MsCl | Methanesulfonyl chloride |
| LAH | Lithium aluminum hydride |
| LDA | Lithium diisopropylamide |
| LiHMDS | Lithium hexamethyldisilazide |
| B₂(Pin)₂ | Bis(pinacolato)diboron |
| NFSI | N-Fluorobenzenesulfonimide |
| MTBE | Methyl tert-butyl ether |
| DMAP | N,N-dimethylpyridin-4-amine |
| DABCO | Triethylenediamine |
| TABF | Tetrabutylammonium fluoride |
| m-CPBA | 3-Chloroperbenzoic acid |
| NMP | N-methylpyrrolidone |
| rt/RT/R.T | Room temperature |
| min(s) | minute(s) |
| h/hr(s) | hour(s) |
| aq | aqueous |
| Sat. | saturated |
| TLC | Thin layer chromatography |
| Prep - TLC | Preparative thin layer chromatography |
| MOMO | Methoxymethoxyl |
| TIPS | Triisopropylsilyl |
| IPA | Isopropyl alcohol |
| cataCXium A Pd G3 | Methanesulfonato(diadamantyl-n-butylphosphino)-2′-amino-1,1′-bipheny1-2- |
| | yl)palladium(II) |
| 4A MS | 4A Molecular sieve |

The intermediates

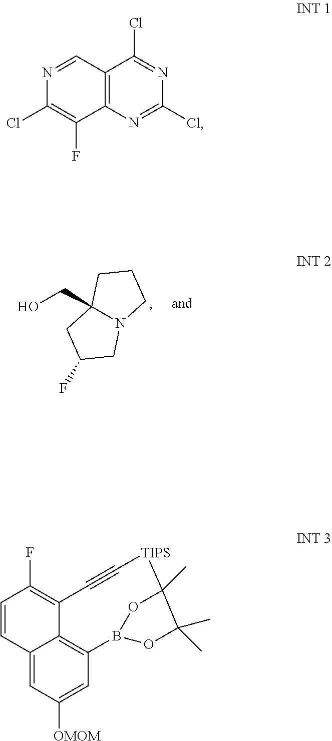

were synthesized using conventional preparation method.

### Example 1

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol trifluoroacetic acid (“Compound 1”)

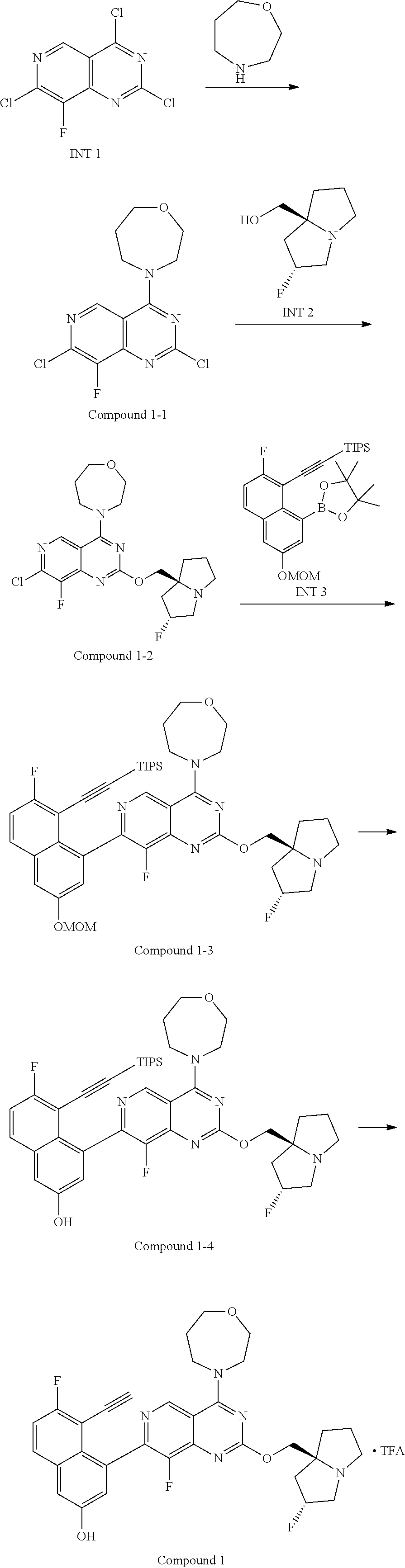

A solution of INT 1 (400 mg, 1.5844 mmol), 1,4-oxazepane (178 mg, 1.7598 mmol) and N,N-diisopropylethylamine (621 mg, 4.8049 mmol) in DCM (10 mL) was stirred at room temperature for 2 h. The solution was diluted with 10% aqueous NaHCO₃ solution (10 mL) and extracted with DCM (10 mL). The organic layer was washed with aqueous NaCl solution, dried over anhydrous Na₂SO₄ and concentrated in vacuum. The residue was purified by Pre-TLC to give a yellow solid as Compound 1-1 (258 mg, 813.5057 mol, 51.3445% yield). MS: m/z: 317 [M+H]⁺.

A solution of Compound 1-1 (127 mg, 400.4466 μmol), INT 2 (106 mg, 665.8262 μmol) and KF (76 mg, 1.3082 mmol) in DMSO (8 mL) was stirred at 85° C. for 20 h under nitrogen atmosphere. The mixture was allowed to cool to room temperature and diluted with saturated aqueous NaHCO₃ solution (20 mL) and extracted with EA (2×20 mL). The organic layer was washed with aqueous NaCl solution (20 mL) then dried over anhydrous Na₂SO₄ and concentrated in vacuum. The residue was purified by Pre-TLC to give Compound 1-2 (165 mg, 375.0974 μmol, 93.6698% yield). MS: m/z: 440 [M+H]⁺.

A solution of Compound 1-2 (0.165 g, 375.0972 μmol), toluene (5 mL), INT 3 (208 mg, 405.8263 μmol), cataCXium A Pd G₃ (31 mg, 42.5666 μmol), potassium phosphate (247 mg, 1.1636 mmol) and water (1 mL) was stirred at 105° C. for 3 h under nitrogen atmosphere. The mixture was allowed to cool to room temperature and diluted with saturated aqueous NaHCO₃ solution (10 mL) and extracted with EA (2×10 mL). The organic layer was washed with 10 mL aqueous NaCl solution then dried over anhydrous Na₂SO₄ and concentrated in vacuum. The residue was purified by Pre-TLC to give Compound 1-3 (189 mg, 239.2410 μmol, 63.7811% yield). MS: m/z: 790 [M+H]⁺.

A solution of Compound 1-3 (0.189 g, 239.2410 μmol) and HCl (4M in dioxane, 1 mL) in DCM (5 mL) was stirred at room temperature for 1 h. The solution was diluted with 10% aqueous NaHCO₃ solution (10 mL) and extracted with DCM (10 mL). The organic layer was washed with saturated aqueous NaCl solution, dried over anhydrous Na₂SO₄ and concentrated in vacuum to give crude Compound 1-4 (178.46 mg, 239.2398 mol, 99.9995% yield). MS: m/z: 746 [M+H]⁺.

A solution of Compound 1-4 (178.46 mg, 239.2398 μmol) and CsF (220 mg, 1.4483 mmol) in DMF (5 mL). The reaction mixture was stirred for 20 h at 40° C. under nitrogen atmosphere. The solution was diluted with saturated aqueous NaHCO₃ solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 45% B in 40 min at a flow rate of 60 mL/min, 240 nm) to afford the desired product Compound 1 (65 mg, TFA salt). MS: m/z: 590 [M+H]⁺.

### Example 2

### (R)-5-ethynyl-6-fluoro-4-(8-fluoro-2-((1-methylpyrrolidin-2-yl)methoxy)-4-(1,4-oxazepan-4-yl)pyrido[4,3-d]pyrimidin-7-yl)naphthalen-2-ol (“Compound 2”)

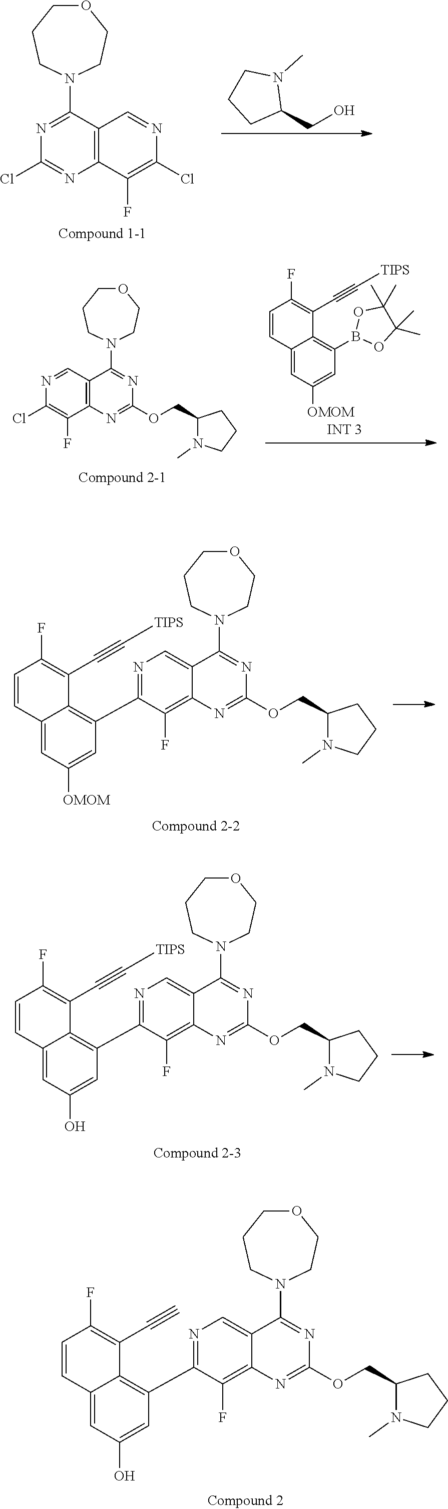

A solution of Compound 1-1 (0.34 g, 1.0720 mmol), (R)-(1-methylpyrrolidin-2-yl)methanol (0.21 g, 1.8232 mmol) and KF (0.36 g, 6.1577 mmol) in DMSO (5 mL) was stirred at 95° C. for 20 hours under nitrogen atmosphere. The mixture was cooled to room temperature and diluted with saturated NaHCO₃ aqueous solution (10 mL) and extracted with EA (30 mL×2). The organic layer was washed with NaCl aqueous solution (20 mL), dried over anhydrous Na₂SO₄, filtered and concentrated in vacuum. The residue was purified by Pre-TLC to give Compound 2-1 (0.14 g, 0.3537 mmol, 32.9907% yield). MS: m/z: 396 [M+H]⁺.

To a solution of Compound 2-1 (0.14 g, 0.3537 mmol) in toluene (5 mL) and water (1 mL), INT 3 (0.28 g, 0.5463 mmol), cataCXium A Pd G3 (126 mg, 173.0128 μmol) and cesium carbonate (450 mg, 1.2430 mmol) were added. The reaction mixture was stirred at 100° C. for 18 hours under nitrogen atmosphere. The mixture was cooled to room temperature, diluted with saturated NaHCO₃ aqueous solution (10 mL) and extracted with EA (30 mL×2). The organic layer was washed with NaCl aqueous solution (20 mL), dried over anhydrous Na₂SO₄, filtered and concentrated in vacuum. The residue was purified by Pre-TLC to give Compound 2-2 (0.14 g, 0.1823 mmol, 52.6788% yield). MS: m/z: 746 [M+H]⁺.

A solution of Compound 2-2 (0.14 g, 0.1823 mmol) and HCl (4M in 1,4-dioxane, 1 mL) in DCM (5 mL) was stirred at room temperature for 1 h. The solution was diluted with 10% NaHCO₃ aqueous solution (8 mL), and extracted with DCM (20 mL×2). The organic layer was washed with NaCl aqueous solution (20 mL), dried over anhydrous Na₂SO₄, filtered and concentrated in vacuum to give crude Compound 2-3 (142 mg, 202.3027 μmol). MS: m/z: 702 [M+H]⁺.

A solution of Compound 2-3 (142 mg, 202.3027 μmol) and CsF (381.4 mg, 2.518 mmol) in DMF (5 mL) was stirred for 20 hours at room temperature under nitrogen atmosphere. The solution was diluted with saturated NaHCO₃ aqueous solution (5 mL) and extracted with EA (20 mL×2). The organic layer was washed with NaCl aqueous solution (20 mL), dried over anhydrous Na₂SO₄, filtered and concentrated in vacuum. The residue was purified by Prep-HPLC (C₁₈ column, phase A: 0.05% NH₃·H₂O in water, phase B: CH₃CN, Gradient: 30% B to 75% B in 36 min at a flow rate of 70 mL/min, 230 nm) to give Compound 2 (15.1 mg). MS: m/z: 546 [M+H]⁺.

### Example 3

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl acetate (“Compound 3”)

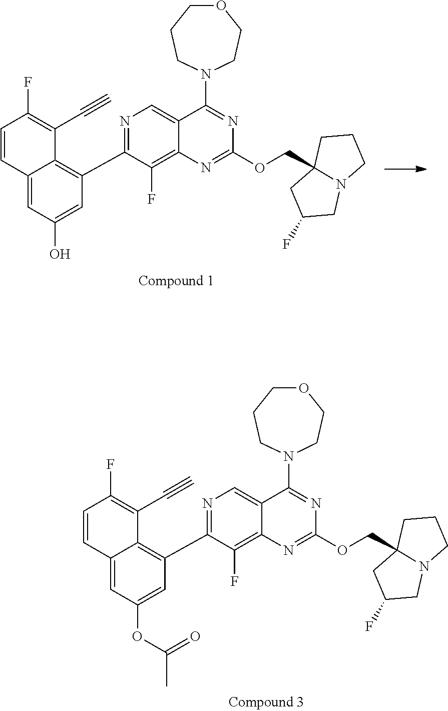 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(C)=O)cc(-c3ncc4c(N5CCCOCC5)nc(OC[C@@]56CCCN5C[C@H](F)C6)nc4c3F)c12]

A solution of Compound 1 (84 mg, 0.14 mmol), acetic anhydride (26 mg, 0.25 mmol) and pyridine (33 mg, 0.42 mmol) in DCM (5 mL) was stirred for 4 hours at room temperature. The reaction mixture was diluted with water (50 mL), extracted with DCM (20 mL), dried over anhydrous Na₂SO₄, filtered and concentrated under vacuum. The residue was purified by pre-HPLC (Daisogel C18, 50 mm×250 mm, 10 um; phase A: 10 mM NH₄HCO₃ in water, phase B: CH₃CN, Gradient: 20% B to 65% B in 45 min at a flow rate of 60 mL/min, 230 nm) to freeze-dried to give Compound 3 (18.9 mg, yield: 21.00%). MS: m/z: 632 [M+H]⁺.

### Example 4

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl dimethylcarbamate (“Compound 4”)

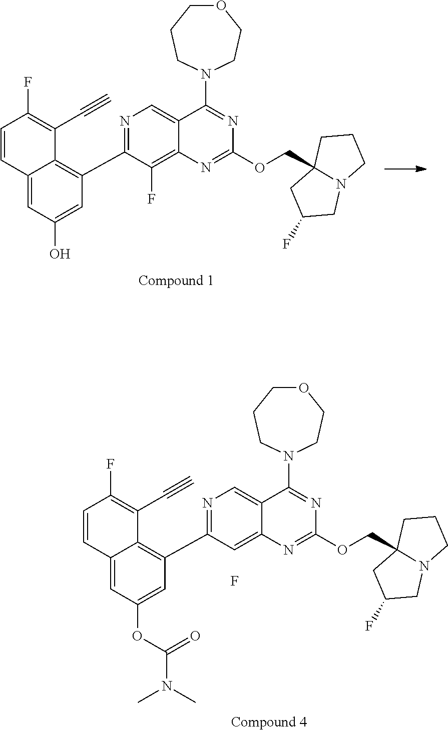

Dimethylcarbamoyl chloride (40 mg, 0.38 mmol) was added to a solution of Compound 1 (82 mg, 0.14 mmol), K₂CO₃ (84 mg, 0.60 mmol) and pyridine (1 mL) in acetonitrile (3 mL). The resulting mixture was stirred overnight at room temperature. The reaction mixture was diluted with water (30 mL) and extracted with DCM (2×30 mL). The organic layers were combined, dried over anhydrous Na₂SO₄, filtered and concentrated under vacuum. The residue was purified by pre-HPLC (Daisogel C18, 50 mm×250 mm, 10 um; phase A: 10 mM NH₄HCO₃ in water, phase B: CH₃CN, Gradient: 35% B to 65% B in 30 min at a flow rate of 60 mL/min, 230 nm) to freeze-dried to give Compound 4 (29.5 mg, yield: 32.10%). MS: m/z: 661 [M+H]⁺.

### Example 5

### ethyl (5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl) carbonate (“Compound 5”)

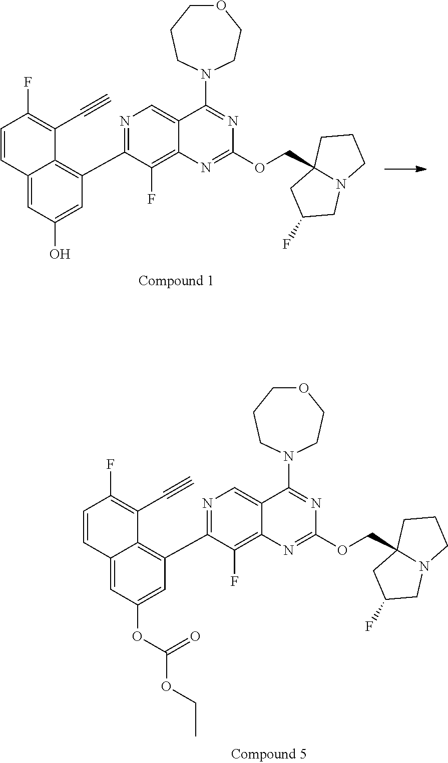 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)OCC)cc(-c3ncc4c(N5CCCOCC5)nc(OC[C@@]56CCCN5C[C@H](F)C6)nc4c3F)c12]

To a solution of ethyl chloroformate (13 mg, 0.12 mmol) in acetonitrile (3 mL) was added to a solution of Compound 1 (81 mg, 0.14 mmol), K₂CO₃ (37 mg, 0.27 mmol) and pyridine (42 mg, 0.53 mmol) in acetonitrile (5 mL) dropwise. The resulting mixture was stirred for 1 hour at room temperature. The reaction mixture was diluted with water (30 mL) and extracted with EtOAc (30 mL). The organic layers were combined, dried over anhydrous Na₂SO₄, filtered and concentrated under vacuum. The residue was purified by pre-HPLC (Daisogel C18, 50 mm×250 mm, 10 um; phase A: 10 mM NH₄HCO₃ in water, phase B: CH₃CN, Gradient: 35% B to 69% B in 35 min at a flow rate of 60 mL/min, 230 nm) to freeze-dried to give Compound 5 (31.9 mg, yield: 35.09%). MS: m/z: 662 [M+H]⁺.

### Example 6

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl palmitate trifluoroacetic acid (“Compound 6”)

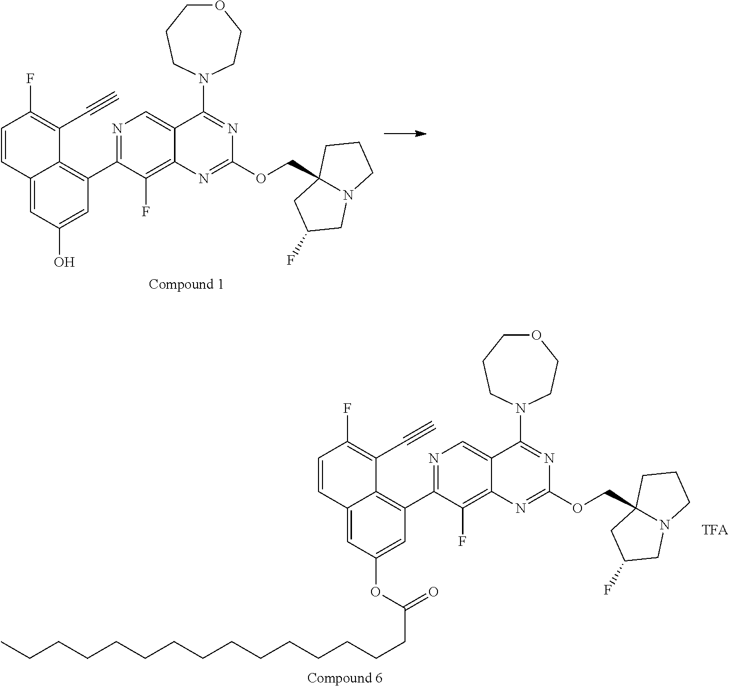 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)CCCCCCCCCCCCCCC)cc(-c3ncc4c(N5CCCOCC5)nc(OC[C@@]56CCCN5C[C@H](F)C6)nc4c3F)c12 ; "TFA"]

Palmitic acid (47 mg, 0.18 mmol) in SOCl₂ (3 mL) was stirred for 1 hour at 80° C. The resulting mixture was concentrated under vacuum. The residue was dissolved in DCM (1.5 mL), and then was added to a solution of Compound 1 (101 mg, 0.17 mmol) and DIEA (1 mL) in DCM (5 mL) dropwise. The resulting mixture was stirred for 1 hour at room temperature. The reaction mixture was diluted with brine (40 mL) and extracted with DCM (20 mL). The organic layers were combined, dried over anhydrous Na₂SO₄, filtered and concentrated under vacuum. The residue was purified by pre-HPLC (Daisogel C18, 50 mm×250 mm, 10 um; phase A: 0.1% TFA in water, phase B: CH₃CN, Gradient: 25% B to 86% B in 61 min at a flow rate of 60 mL/min, 230 nm) to freeze-dried to give Compound 6 (105.5 mg, TFA salt, yield: 65.38%). MS: m/z: 828 [M+H]⁺.

### Example 7

### 4-(4-(2-oxa-6-azabicyclo[5.1.0]octan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 7”)

### 4-(4-((1S,7R)-2-oxa-6-azabicyclo[5.1.0]octan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 7A or Compound 7B”)

### 4-(4-((1R,7S)-2-oxa-6-azabicyclo[5.1.0]octan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 7B or Compound 7A”)

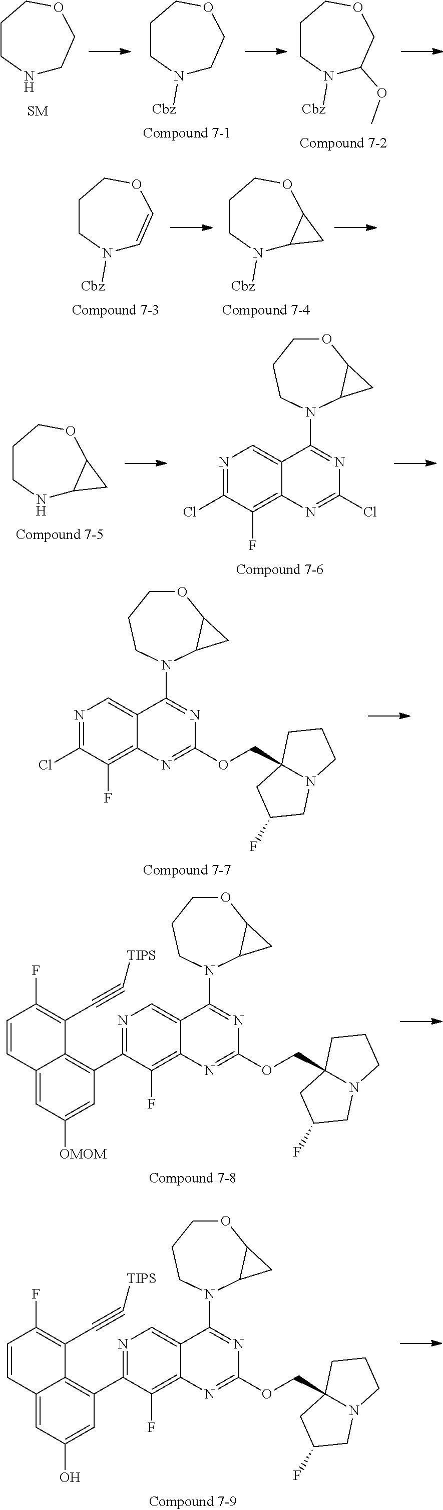

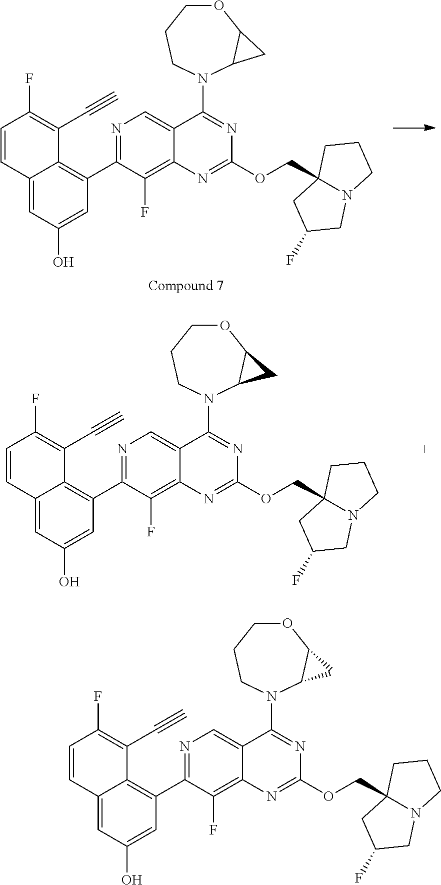

K₂CO₃ (18.0 g, 130 mmol) was added into THF (40.0 mL), and then SM (4.40 g, 43.5 mmol) and Cbz-Cl (8.91 g, 52.2 mmol) were added. The mixture was stirred at 20° C. for 16 hrs. After that, water (20.0 mL) was added, stirred at 20° C. for 1 hr and extracted with EtOAc (30.0 mL×3). The organic layer was collected, dried over with anhydrous Na₂SO₄ and concentrated in vacuum. The residue was purified by column chromatography (SiO₂, petroleum ether/ethyl acetate=30/1 to 2/1) to give Compound 7-1 (7.49 g, 31.8 mmol).

¹H NMR: (400 MHz, CDCl₃) δ 7.32-7.37 (m, 5H), 5.15 (s, 2H), 3.70-3.75 (m, 4H), 3.58-3.62 (m, 4H), 1.87-2.04 (m, 2H).

Compound 7-1 (2.30 g, 9.78 mmol) was dissolved in a 0.025 M solution of Et₄NOTs (1.47 g, 4.89 mmol) in MeOH (40 mL). The reaction mixture was electrolysis with C (+)|C (−) electrodes at constant current 28 mA (10 F/mol, 10 mA/cm²). The mixture was stirred at 25° C. for 16 h and concentrated in vacuum. The residue was purified by column chromatography (SiO₂, petroleum ether/ethyl acetate=30/1 to 5/1) to give Compound 7-2 (4.01 g, 15.1 mmol).

Compound 7-2 (2.60 g, 9.80 mmol) was added into DCM (18.0 mL) and then TMSOTf (2.61 g, 11.7 mmol, 2.13 mL) and DIEA (1.52 g, 11.7 mmol, 2.05 mL) were added. The mixture was stirred at 20° C. for 2 h. After that, the mixture was washed with NaHCO₃ aqueous solution (20.0 mL×3) and washed with brine (20 mL×3). The organic layer was collected, dried over with anhydrous Na₂SO₄ and concentrated in vacuum. The residue was purified by column chromatography (SiO₂, Petroleum ether/Ethyl acetate=30/1 to 2/1) to give 1-3 (0.376 g, 1.61 mmol).

¹H NMR: (400 MHz, CDCl₃) δ 7.32-7.38 (m, 5H), 5.75-5.96 (m, 2H), 5.19 (s, 2H), 4.08-4.11 (m, 2H), 3.85-3.88 (m, 2H), 1.98-2.02 (m, 2H).

Compound 7-3 (0.376 g, 1.61 mmol) was added in DCM (5.00 mL). ZnEt₂ (1 M, 4.03 mL) was added to the solution at 20° C. and stirred at 20° C. for 0.5 h. A solution of MeI₂ (1.73 g, 6.45 mmol, 520 μL) in DCM (2.00 mL) was added to the mixture dropwise and then stirred at 20° C. for 12 h. The reaction mixture was poured into saturated NH₄Cl aqueous solution (15.0 mL). The organic layer was separated, washed with brine (15.0 mL×3), dried with anhydrous Na₂SO₄ and concentrated in vacuum. The residue was purified by column chromatography (petroleum ether/ethyl acetate=30/1 to 2/1) to give Compound 7-4 (0.36 g, 1.45 mmol). MS: m/z: 248 [M+1]⁺.

¹H NMR: (400 MHz, CDCl₃) δ 7.33-7.37 (m, 5H), 5.11-5.21 (m, 2H), 3.94-4.19 (m, 2H), 3.64-3.69 (m, 1H), 3.42 (s, 1H), 3.11-3.16 (m, 1H), 2.44 (s, 1H), 1.75-1.92 (m, 2H), 1.14-1.22 (m, 2H).

A solution of Compound 7-4 (0.315 g, 1.2738 mmol) and Pd/C (101 mg) in MeOH (10 mL) was stirred in H₂ at room temperature for 2.5 h. The mixture was filtered and concentrated in vacuum to Compound 7-5 (45 mg, 397.6776 μmol). MS: m/z: 114 [M+1]⁺.

Compound 7-5 (0.045 g, 397.6776 μmol) was added into a solution of 2,4,7-trichloro-8-fluoropyrido[4,3-d]pyrimidine (101 mg, 400.0629 mmol) and DIEA (150 mg, 1.1606 mmol) in DCM (10 mL). The mixture was stirred at rt for 2 h. The solution was diluted with 10% citric solution (10 mL) and extracted with DCM (10 mL). The organic layer was washed with NaCl aqueous solution, dried over anhydrous Na₂SO₄ and concentrated in vacuum to give Compound 7-6 (142 mg, 431.4056 μmol). MS: m/z: 329 [M+1]⁺.

A solution of Compound 7-6 (0.142 g, 431.4056 μmol), INT 2 (111 mg, 697.2331 μmol) and KF (86 mg, 1.4803 mmol) in DMSO (10 mL) was stirred at 100° C. for 20 h under nitrogen atmosphere. The mixture was cooled to room temperature, diluted with saturated NaCl aqueous solution (15 mL) and extracted with EA (15 mL). The organic layer was washed with NaCl aqueous solution (15 mL) then dried over anhydrous Na₂SO₄ and concentrated in vacuum. The residue was purified by Pre-TLC to give Compound 7-7 (89 mg, 196.9478 μmol). MS: m/z: 452 [M+1]⁺.

A solution of Compound 7-7 (0.089 g, 196.9480 μmol), INT 3 (155 mg, 302.4186 μmol), cataCXium A Pd G3 (17 mg, 23.3430 μmol), cesium carbonate, (213 mg, 653.7374 μmol), toluene (10 mL) and water (2 mL) was stirred at 100° C. overnight under nitrogen atmosphere. The mixture was allowed to cool to room temperature, diluted with saturated NaCl aqueous solution (15 mL) and extracted with EA (15 mL). The organic layer was washed with 15 mL NaCl aqueous solution, then dried over anhydrous Na₂SO₄ and concentrated in vacuum. The residue was purified by Pre-TLC to give Compound 7-8 (146 mg, 182.0428 μmol). MS: m/z: 802 [M+1]⁺.

A solution of Compound 7-8 (0.146 g, 182.0428 μmol) and HCl (4M in dioxane, 1 mL) in DCM (9 mL) was stirred at room temperature for 1 h. The solution was diluted with 10% NaHCO₃ aqueous solution (20 mL). The organic layer was washed with saturated NaCl aqueous solution, dried over anhydrous Na₂SO₄ and concentrated in vacuum to give crude Compound 7-9 (129 mg, 170.1944 μmol). MS: m/z: 758 [M+1]⁺.

A solution of Compound 7-9 (0.129 g, 170.1944 μmol) and CsF (130 mg, 855.8070 μmol) in DMF (8 mL) was stirred for 20 hours at 40° C. under nitrogen atmosphere. The solution was diluted with H₂O (10 mL) and extracted with EA (10 mL×2). The organic layer was dried over anhydrous Na₂SO₄ and concentrated under vacuum. The residue was purified by Prep-HPLC (C18 column, A: 0.1% TFA in water, B: CH₃CN, Gradient: 15% B to 35% B in 34 min at a flow rate of 60 mL/min, 235 nm). The eluent was adjusted to pH 8 and the acetonitrile in the eluent was concentrated. The resulting aqueous phase was extracted with DCM (50 mL), and the organic phase was dried and concentrated then freeze-dried to afford Compound 7 (36 mg, 59.8387 μmol).

The Compound 7 was separated by Prep-HPLC-Gilson with the following conditions: Column, CHIRAL ART Cellulose-SA column (2 cm×25 cm, 5 um); Mobile phase, Hex/EtOH (60:40); Flowing rate, 20 ml/min. This results in Compound 7A (the first eluting isomer, Retention Time 4.535 min) and Compound 7B (the second eluting isomer, Retention Time 5.347 min) MS: m/z: 602 [M+1]⁺.

### Example 8

### 5-ethynyl-6-fluoro-4-(8-fluoro-2-(((2R,7aS)-2-fluorotetrahydro-1H-pyrrolizin-7a(5H)-yl)methoxy)-4-((S)-2-methyl-1,4-oxazepan-4-yl)pyrido[4,3-d]pyrimidin-7-yl)naphthalen-2-ol trifluoroacetic acid (“Compound 8A or Compound 8B”)

### 5-ethynyl-6-fluoro-4-(8-fluoro-2-(((2R,7aS)-2-fluorotetrahydro-1H-pyrrolizin-7a(5H)-yl)methoxy)-4-((R)-2-methyl-1,4-oxazepan-4-yl)pyrido[4,3-d]pyrimidin-7-yl)naphthalen-2-ol trifluoroacetic acid (“Compound 8B or Compound 8A”)

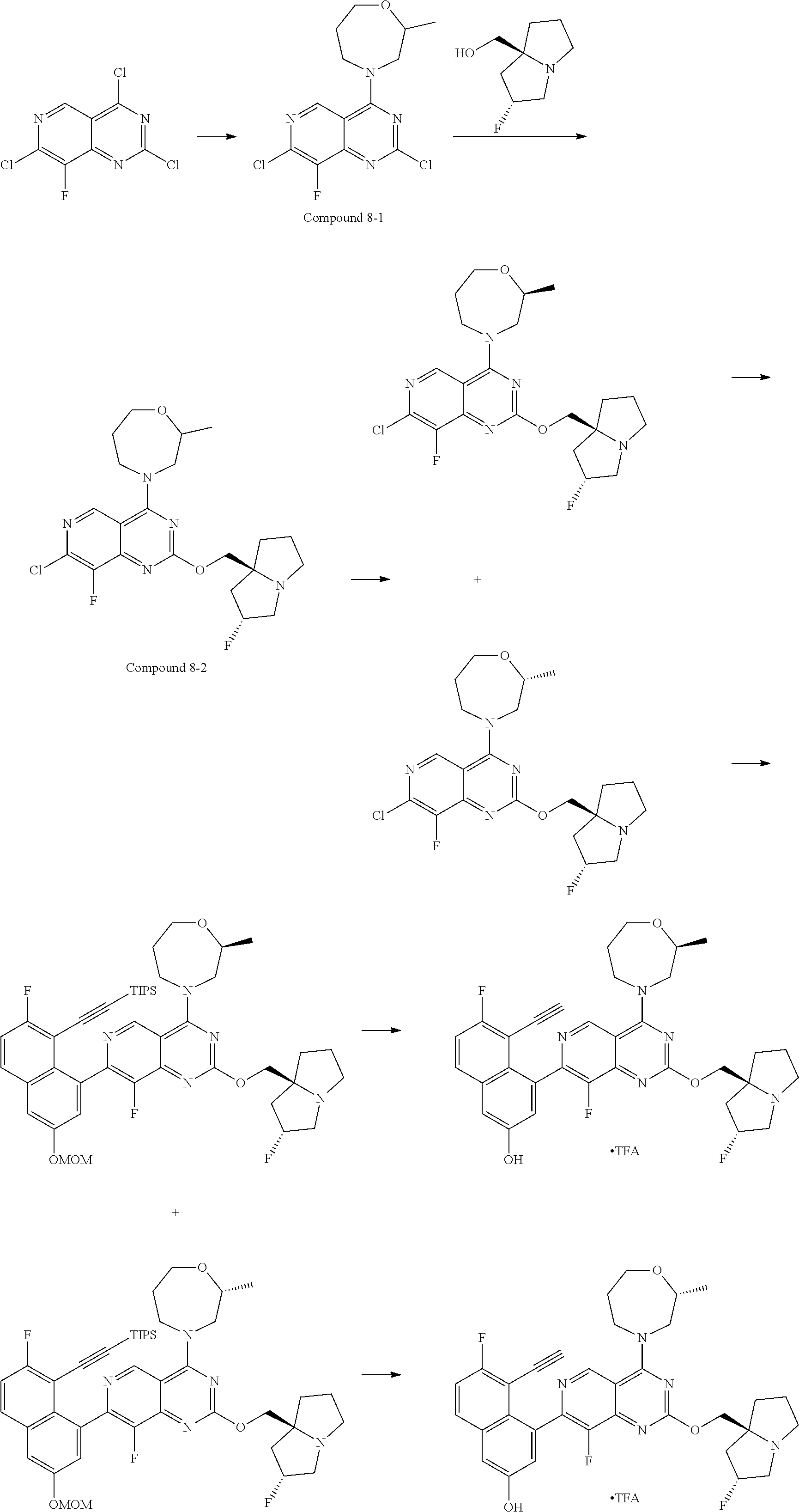

A solution of INT 1 (469 mg, 1.8577 mmol), 2-methyl-1,4-oxazepane hydrochloride (253 mg, 1.6685 mmol) and DIEA (704 mg, 5.4456 mmol) in DCM (8 mL) was stirred at room temperature for 2 h. The solution was diluted with 10% NaHCO₃ aqueous solution (10 mL) and extracted with DCM (10 mL). The organic layer was washed with NaCl aqueous solution, dried over anhydrous Na₂SO₄ and concentrated in vacuum to give Compound 8-1 (581 mg, 1.6140 mmol, 86.8821% yield). MS: m/z: 331 [M+H]⁺.

A solution of Compound 8-1 (581 mg, 1.6140 mmol), INT 2 (322 mg, 2.0226 mmol) and KF (327 mg, 5.6285 mmol) in DMSO (10 mL) was stirred at 85° C. for 20 h under nitrogen atmosphere. The mixture was allowed to cool to room temperature, diluted with saturated NaHCO₃ aqueous solution (20 mL) and extracted with EA (2×20 mL). The organic layer was washed with NaCl aqueous solution (20 mL) then dried over anhydrous Na₂SO₄ and concentrated in vacuum. The residue was purified by Pre-TLC to give Compound 8-2 (267 mg, 482.3400 μmol, 27.4936% yield). MS: m/z: 454 [M+H]f.

Compound 8-2 was separated by Prep-HPLC-Gilson with the following conditions: Column, CHIRAL ART Cellulose-SC column (2 cm×25 cm, 5 um); Mobile phase, Hex (0.1% isopropylamine)/EtOH (50:50); Flowing rate: 20 ml/min. This results in the first eluting isomer Compound 8-3A (59 mg, Retention Time 12.548 min) and the second eluting isomer Compound 8-3B (71 mg, Retention Time 14.11 min) respectively.

A solution of Compound 8-3A (59 mg, 129.9811 μmol), toluene (5 mL), INT 3 (102 mg, 199.0110 μmol), cataCXium A Pd G3 (14 mg, 19.2236 μmol), Cs₂CO₃ (131 mg, 402.0638 μmol) and water (1 mL) was stirred at 100° C. overnight under nitrogen atmosphere. The mixture was allowed to cool to room temperature, diluted with saturated NaHCO₃ aqueous solution (10 mL) and extracted with EA (2×10 mL). The organic layer was washed with NaCl aqueous solution (10 mL), then dried over anhydrous Na₂SO₄ and concentrated in vacuum. The residue was purified by Pre-TLC to give Compound 8-4A (20 mg, 24.8748 μmol, 19.14% yield). MS: m/z: 804 [M+H]⁺.

A solution of Compound 8-4A (20 mg, 24.8748 μmol) and HCl (4M in 1,4-dioxane, 0.8 mL) in ACN (3 mL) was stirred at room temperature for 1 h. The solution was diluted with 10% NaHCO₃ aqueous solution (10 mL) and extracted with EA (10 mL). The organic layer was washed with saturated NaCl aqueous solution, dried over anhydrous Na₂SO₄ and concentrated in vacuum. The residue in DMF (4 mL) was added CsF (0.13 g, 855.8070 μmol). The reaction mixture was stirred for 2 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 35% B in 35 min at a flow rate of 40 mL/min, 230 nm) to give Compound 8A (13.5 mg, TFA salt). MS: m/z: 604 [M+H]⁺.

A solution of Compound 8-3B (71 mg, 156.4179 μmol), toluene (5 mL), INT 3 (120 mg, 234.1305 μmol), cataCXium A Pd G3 (15 mg, 20.5968 μmol), Cs₂CO₃ (158 mg, 484.9320 μmol) and water (1 mL) was stirred at 100° C. overnight under nitrogen atmosphere. The mixture was allowed to cool to room temperature, diluted with saturated NaHCO₃ aqueous solution (10 mL) and extracted with EA (2×10 mL). The organic layer was washed with NaCl aqueous solution (10 mL), dried over anhydrous Na₂SO₄ and concentrated in vacuum. The residue was purified by Pre-TLC to give Compound 8-4B (71 mg, 88.3057 μmol, 56.46% yield). MS: m/z: 804 [M+H]⁺.

A solution of Compound 8-4B (71 mg, 88.3057 μmol) and HCl (4M in 1,4-dioxane, 0.9 mL) in ACN (3 mL) was stirred at room temperature for 1 h. The solution was diluted with 10% NaHCO₃ aqueous solution (10 mL) and extracted with EA (10 mL). The organic layer was washed with saturated NaCl aqueous solution, dried over anhydrous Na₂SO₄ and concentrated in vacuum. The residue in DMF (4 mL) was added CsF (0.23 g, 1.5141 mmol). The reaction mixture was stirred for 2 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 40 min at a flow rate of 60 mL/min, 230 nm) to give Compound 8B (19.9 mg, TFA salt). MS: m/z: 604 [M+H]⁺.

### Example 9

### 4-(2-((2,2-difluorotetrahydro-1H-pyrrolizin-7a(5H)-yl)methoxy)-8-fluoro-4-(1,4-oxazepan-4-yl)pyrido[4,3-d]pyrimidin-7-yl)-5-ethynyl-6-fluoronaphthalen-2-ol (“Compound 9”)

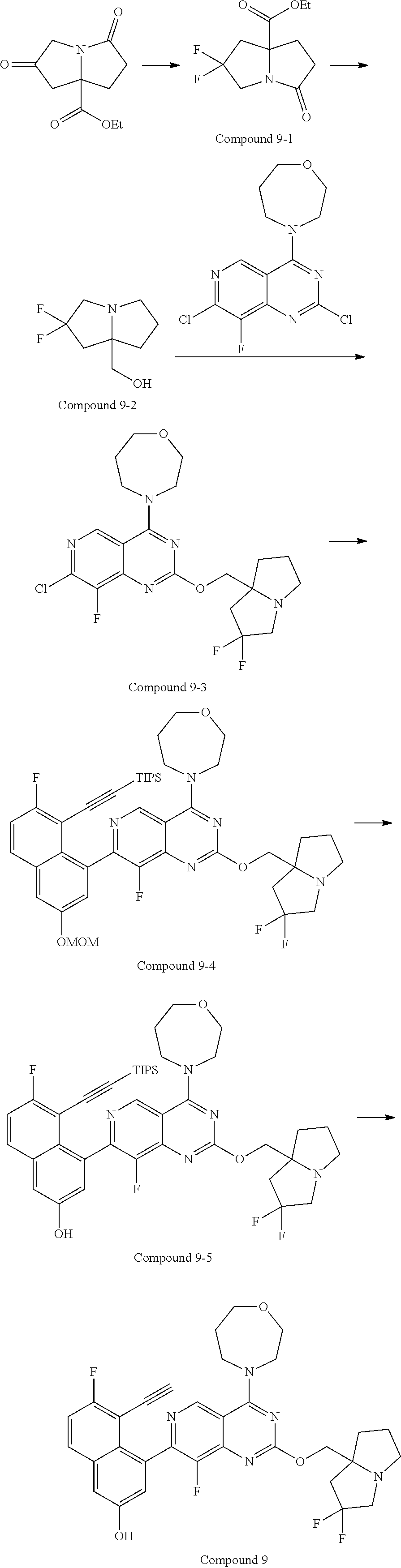

To a solution of ethyl 2,5-dioxotetrahydro-1H-pyrrolizine-7a(5H)-carboxylate (861 mg, 4.0764 mmol) in DCM (20 mL) was added diethylaminosulfur trifluoride (1988 mg, 12.3334 mmol) at 0° C. under nitrogen atmosphere. The reaction mixture was stirred at room temperature for 20 hours. The solution was diluted with saturated NaHCO₃ aqueous solution (20 mL) and extracted with DCM (50 mL). The organic layer was washed with NaCl aqueous solution, dried over anhydrous Na₂SO₄ and concentrated in vacuum. The residue was purified by Pre-TLC to give Compound 9-1 (728 mg, 3.1216 mmol, 76.5775% yield). MS: m/z: 234 [M+H]⁺.

To a solution of Compound 9-1 (720 mg, 3.0873 mmol) in THF (15 mL) was added LiAlH₄ (299 mg, 7.8788 mmol) at 0° C. under nitrogen atmosphere. The reaction mixture was stirred at 70° C. for 2 hours. The solution was diluted with EA (60 mL) and quenched by sodium sulfate decahydrate. The resulting suspension was filtered through celite and concentrated in vacuum to give Compound 9-2 (494 mg, 2.7879 mmol, 90.3030% yield). MS: m/z: 178 [M+H]⁺.

A solution of Compound 9-2 (131 mg, 739.3152 μmol), Compound 1-1 (201 mg, 633.7777 μmol) and KF (426 mg, 7.3326 mmol) in DMSO (6 mL) was stirred at 90° C. for 16 h under nitrogen atmosphere. The mixture was allowed to cool to room temperature, diluted with saturated NaHCO₃ aqueous solution (30 mL) and extracted with EA (50 mL). The organic layer was washed with NaCl aqueous solution, then dried over anhydrous Na₂SO₄ and concentrated in vacuum. The residue was purified by Pre-TLC to give Compound 9-3 (108 mg, 235.8716 μmol, 37.2168% yield). MS: m/z: 458 [M+H]⁺.

A solution of Compound 9-3 (107 mg, 233.6876 μmol), toluene (5 mL), INT 3 (164 mg, 319.9784 μmol), cataCXium A Pd G3 (25 mg, 34.3279 μmol), cesium carbonate (228 mg, 699.7752 μmol) and water (1 mL) was stirred at 100° C. for 16 hours under nitrogen atmosphere. The mixture was allowed to cool to room temperature, diluted with saturated NaHCO₃ aqueous solution (20 mL) and extracted with EA (2×30 mL). The organic layer was washed with NaCl aqueous solution, then dried over anhydrous Na₂SO₄ and concentrated in vacuum. The residue was purified by Pre-TLC to give Compound 9-4 (88 mg, 108.9124 μmol, 46.6060% yield). MS: m/z: 808 [M+H]⁺.

A solution of Compound 9-4 (88 mg, 108.9124 μmol) and HCl (4M in 1,4-dioxane, 0.8 mL) in DCM (5 mL) was stirred at room temperature for 0.5 h. The solution was diluted with 10% NaHCO₃ aqueous solution (20 mL) and extracted with DCM (50 mL). The organic layer was washed with NaCl aqueous solution, dried over anhydrous Na₂SO₄ and concentrated in vacuum to give crude Compound 9-5 (77 mg, 100.7937 μmol, 92.5457% yield). MS: m/z: 764 [M+H]⁺.

A solution of Compound 9-5 (77 mg, 100.7937 μmol) and CsF (88 mg, 579.3315 μmol) in DMF (5 mL) was stirred for 15 hours at 35° C. under nitrogen atmosphere. The solution was diluted with saturated NaHCO₃ aqueous solution (20 mL) and extracted with EA (60 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 36 min at a flow rate of 60 mL/min, 230 nm). The eluent was adjusted to PH 8, acetonitrile was concentrated, aqueous phase was extracted with DCM (60 mL), and the organic phase was dried and concentrated then to freeze-dried to give Compound 9 (23 mg). MS: m/z: 608 [M+H]⁺.

### Example 10

### 5-ethynyl-6-fluoro-4-(8-fluoro-2-((2-fluoro-2-methyltetrahydro-1H-pyrrolizin-7a(5H)-yl)methoxy)-4-(1,4-oxazepan-4-yl)pyrido[4,3-d]pyrimidin-7-yl)naphthalen-2-ol (“Compound 10”)

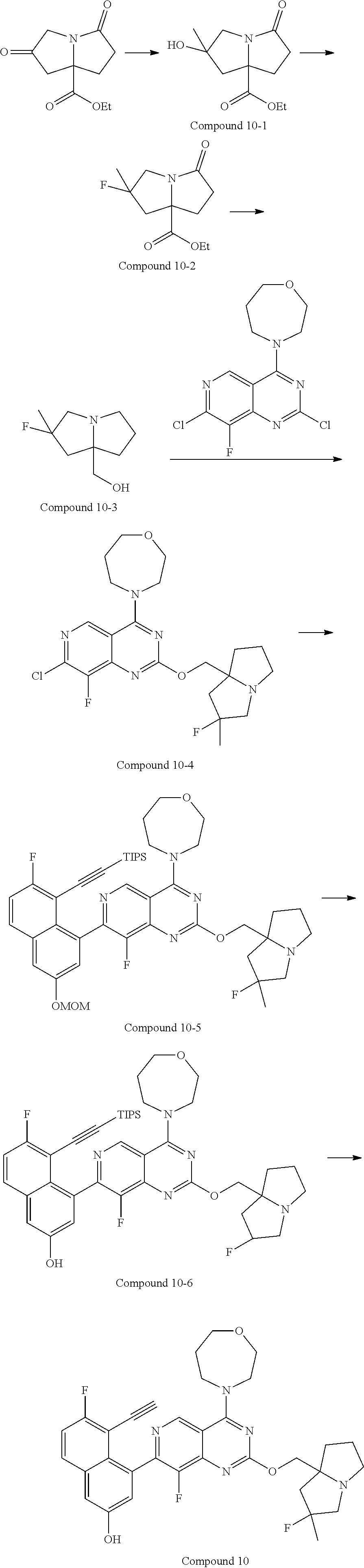

To a solution of ethyl 2,5-dioxotetrahydro-1H-pyrrolizine-7a(5H)-carboxylate (1509 mg, 7.1444 mmol) in THF (40 mL) was added methylenemagnesium bromide (5 mL, 3M in dimethyl ether) at −78° C. under nitrogen atmosphere. The reaction mixture was stirred at −78° C. for 3 hours. The solution was diluted with saturated NH₄Cl aqueous solution (30 mL) and extracted with DCM (2×50 mL). The organic layer was washed with NaCl aqueous solution, dried over anhydrous Na₂SO₄ and concentrated in vacuum. The residue was purified by Pre-TLC to give Compound 10-1 (718 mg, 3.1594 mmol, 44.2224% yield). MS: m/z: 228 [M+H]⁺.

To a solution of Compound 10-1 (672 mg, 2.9570 mmol) in DCM (20 mL) was added diethylaminosulfur trifluoride (493 mg, 3.0585 mmol) at 0° C. under nitrogen atmosphere. The reaction mixture was stirred at room temperature for 18 hours. The solution was diluted with saturated NaHCO₃ aqueous solution (20 mL) and extracted with DCM (2×40 mL). The organic layer was washed with NaCl aqueous solution, dried over anhydrous Na₂SO₄ and concentrated in vacuum. The residue was purified by Pre-TLC to give Compound 10-2 (197 mg, 859.3336 μmol, 29.0609% yield). MS: m/z: 230 [M+H]⁺.

To a solution of Compound 10-2 (197 mg, 859.3336 mol) in THF (8 mL) was added LiAlH₄ (82 mg, 2.1607 mmol) at 0° C. under nitrogen atmosphere. The reaction mixture was stirred at 70° C. for 2 hours. The solution was diluted with EA (20 mL) and quenched by sodium sulfate decahydrate. The resulting suspension was filtered through celite and concentrated in vacuum to give Compound 10-3 (173 mg, crude product). MS: m/z: 174 [M+H]⁺.

A solution of Compound 10-3 (171 mg, 987.1429 μmol), Compound 1-1 (252 mg, 794.5870 μmol) and KF (263 mg, 4.5269 mmol) in DMSO (5 mL) was stirred at 90° C. for 16 h under nitrogen atmosphere. The mixture was allowed to cool to room temperature, diluted with saturated NaHCO₃ aqueous solution (40 mL) and extracted with EA (2×40 mL). The organic layer was washed with NaCl aqueous solution, then dried over anhydrous Na₂SO₄ and concentrated in vacuum. The residue was purified by Pre-TLC to give Compound 10-4 (87 mg, 191.6670 μmol, 24.1216% yield). MS: m/z: 454 [M+H]⁺.

A solution of Compound 10-4 (87 mg, 191.6670 μmol), toluene (5 mL), INT 3 (150 mg, 292.6632 μmol), cataCXium A Pd G3 (19 mg, 26.0892 μmol), cesium carbonate (198 mg, 607.6996 μmol) and water (1 mL) was stirred at 100° C. for 20 hours under nitrogen atmosphere. The mixture was allowed to cool to room temperature, diluted with saturated NaHCO₃ aqueous solution (20 mL) and extracted with EA (50 mL). The organic layer was washed with NaCl aqueous solution, then dried over anhydrous Na₂SO₄ and concentrated in vacuum. The residue was purified by Pre-TLC to give Compound 10-5 (42 mg, 52.2372 μmol, 27.2541% yield). MS: m/z: 804 [M+H]⁺.

A solution of Compound 10-5 (42 mg, 52.2372 μmol) and HCl (4M in 1,4-dioxane, 0.5 mL) in DCM (3 mL) was stirred at room temperature for 0.5 h. The solution was diluted with 10% NaHCO₃ aqueous solution (20 mL) and extracted with DCM (50 mL). The organic layer was washed with NaCl aqueous solution, dried over anhydrous Na₂SO₄ and concentrated in vacuum to give crude Compound 10-6 (24 mg, 31.5801 mol, 60.4552% yield). MS: m/z: 760 [M+H]⁺.

A solution of Compound 10-6 (24 mg, 31.5801 μmol) and CsF (104 mg, 684.6456 μmol) in DMF (3 mL) was stirred for 4 hours at 35° C. under nitrogen atmosphere. The solution was diluted with saturated NaHCO₃ aqueous solution (20 mL) and extracted with EA (2×20 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 34% B in 24 min at a flow rate of 40 mL/min, 230 nm). The eluent was adjusted to pH 8 and the acetonitrile in the eluent was concentrated. The resulting aqueous phase was extracted with DCM (60 mL), and the organic phase was dried and concentrated then freeze-dried to give Compound 10 (11 mg). MS: m/z: 604 [M+H]⁺.

### Example 11

### 5-ethynyl-6-fluoro-4-(8-fluoro-2-(((2R,7aS)-2-methoxytetrahydro-1H-pyrrolizin-7a(5H)-yl)methoxy)-4-(1,4-oxazepan-4-yl)pyrido[4,3-d]pyrimidin-7-yl)naphthalen-2-ol (“Compound 11”)

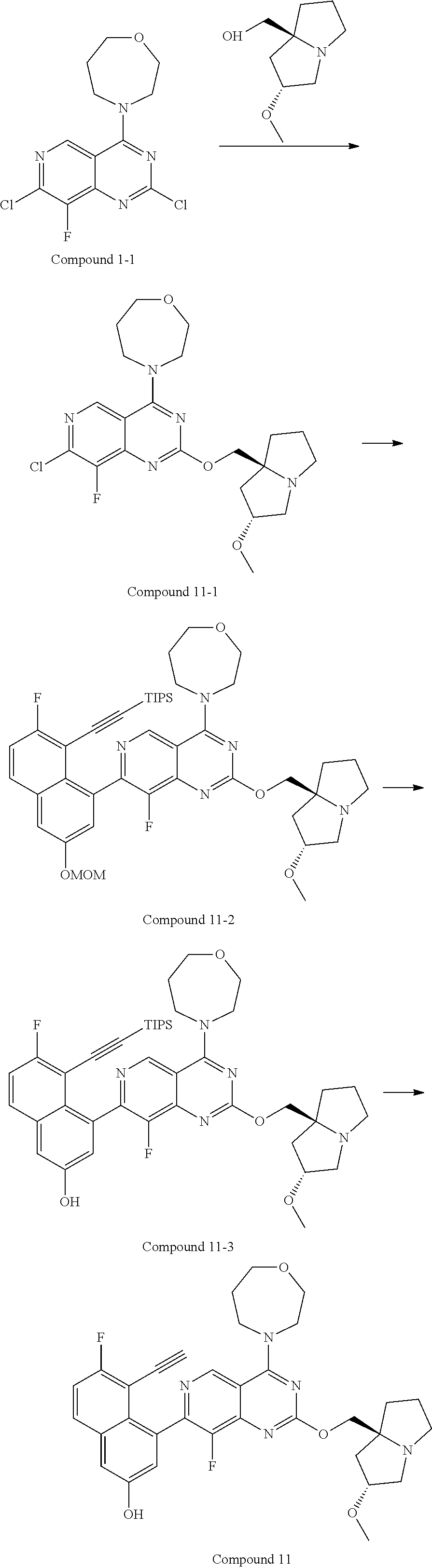

A solution of Compound 1-1 (160 mg, 504.4997 μmol), ((2R,7aS)-2-methoxytetrahydro-1H-pyrrolizin-7a(5H)-yl)methanol (105 mg, 613.1885 μmol) and KF (190 mg, 3.2704 mmol) in DMSO (5 mL) was stirred at 90° C. for 16 h under nitrogen atmosphere. The mixture was allowed to cool to room temperature, diluted with saturated NaHCO₃ aqueous solution (30 mL) and extracted with EA (2×30 mL). The organic layer was washed with NaCl aqueous solution, then dried over anhydrous Na₂SO₄ and concentrated in vacuum. The residue was purified by Pre-TLC to give Compound 11-2 (97 mg, 214.6392 μmol, 42.5450% yield). MS: m/z: 452 [M+H]⁺.

A solution of Compound 11-2 (96 mg, 212.4265 μmol), toluene (5 mL), INT 3 (174 mg, 339.4890 μmol), cataCXium A Pd G3 (18 mg, 24.7161 gmol), cesium carbonate (218 mg, 669.0833 gmol) and water (1 mL) was stirred at 100° C. for 15 hours under nitrogen atmosphere. The mixture was allowed to cool to room temperature, diluted with saturated NaHCO₃ aqueous solution (20 mL) and extracted with EA (50 mL). The organic layer was washed with NaCl aqueous solution, then dried over anhydrous Na₂SO₄ and concentrated in vacuum. The residue was purified by Pre-TLC to give Compound 11-3 (67 mg, 83.5376 μmol, 39.3254% yield). MS: m/z: 802 [M+H]⁺.

A solution of Compound 11-3 (67 mg, 83.5376 μmol) and HCl (4M in 1,4-dioxane, 1 mL) in DCM (5 mL) was stirred at room temperature for 0.5 h. The solution was diluted with 10% NaHCO₃ aqueous solution (20 mL) and extracted with DCM (30 mL). The organic layer was washed with NaCl aqueous solution, dried over anhydrous Na₂SO₄ and concentrated in vacuum to give crude Compound 11-4 (66 mg, 87.0734 μmol, 104.2325% yield). MS: m/z: 758 [M+H]⁺.

A solution of Compound 11-4 (66 mg, 87.0734 μmol) and CsF (76 mg, 500.3180 μmol) in DMF (5 mL) was stirred for 15 hours at 35° C. under nitrogen atmosphere. The solution was diluted with saturated NaHCO₃ aqueous solution (20 mL) and extracted with EA (2×30 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 39% B in 34 min at a flow rate of 60 mL/min, 230 nm). The eluent was adjusted to pH 8 and the acetonitrile in the eluent was concentrated. The resulting aqueous phase was extracted with DCM (60 mL), and the organic phase was dried and concentrated then freeze-dried to afford Compound 11 (21 mg). MS: m/z: 602 [M+H]⁺.

### Example 12

### 5-ethynyl-6-fluoro-4-(8-fluoro-4-(6-fluoro-1,4-oxazepan-4-yl)-2-(((2R,7aS)-2-fluorotetrahydro-1H-pyrrolizin-7a(5H)-yl)methoxy)pyrido[4,3-d]pyrimidin-7-yl)naphthalen-2-ol trifluoroacetic acid (“Compound 12”)

### 5-ethynyl-6-fluoro-4-(8-fluoro-4-((S)-6-fluoro-1,4-oxazepan-4-yl)-2-(((2R,7aS)-2-fluorotetrahydro-1H-pyrrolizin-7a(5H)-yl)methoxy)pyrido[4,3-d]pyrimidin-7-yl)naphthalen-2-ol (“Compound 12A” or “Compound 12B”)

### 5-ethynyl-6-fluoro-4-(8-fluoro-4-((R)-6-fluoro-1,4-oxazepan-4-yl)-2-(((2R,7aS)-2-fluorotetrahydro-1H-pyrrolizin-7a(5H)-yl)methoxy)pyrido[4,3-d]pyrimidin-7-yl)naphthalen-2-ol (“Compound 12B” or “Compound 12A”)

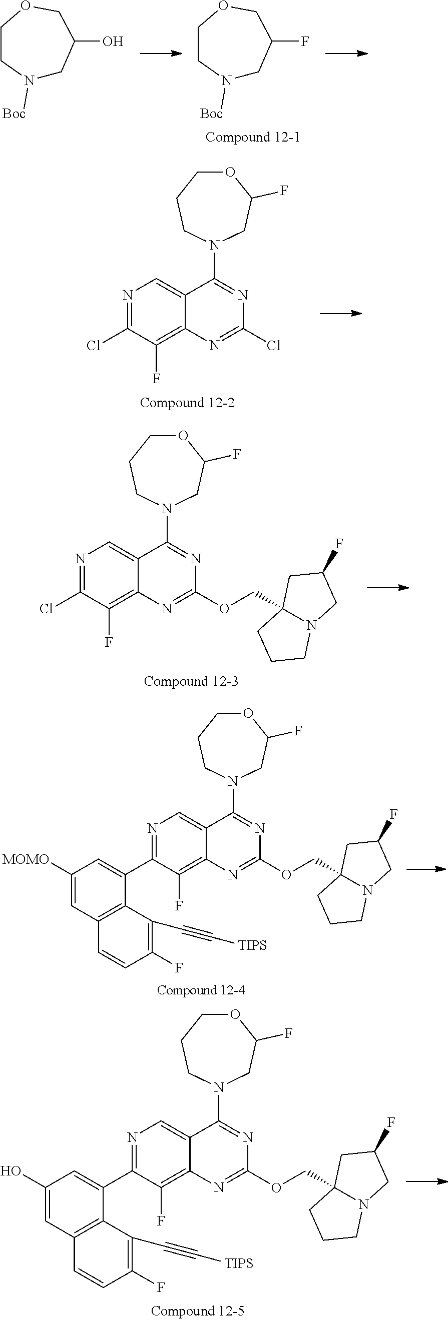

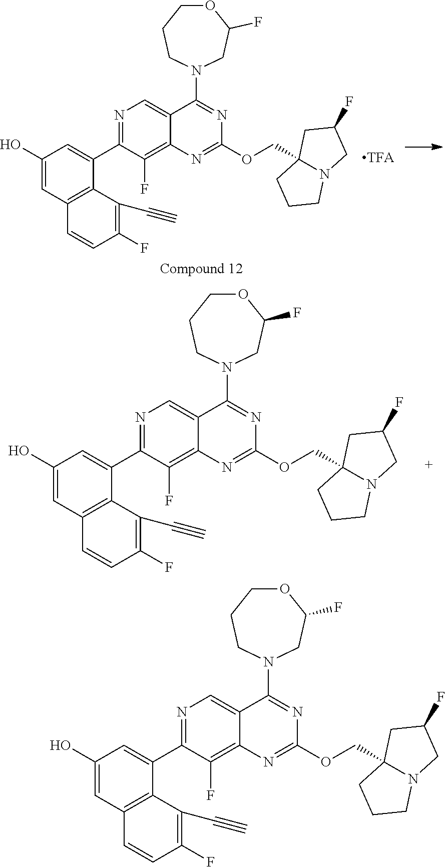 [SMILES: C#Cc1c(F)ccc2cc(O)cc(-c3ncc4c(N5CCCOC(F)C5)nc(OC[C@@]56CCCN5C[C@H](F)C6)nc4c3F)c12 ; C#Cc1c(F)ccc2cc(O)cc(-c3ncc4c(N5CCCO[C@@H](F)C5)nc(OC[C@@]56CCCN5C[C@H](F)C6)nc4c3F)c12 ; C#Cc1c(F)ccc2cc(O)cc(-c3ncc4c(N5CCCO[C@H](F)C5)nc(OC[C@@]56CCCN5C[C@H](F)C6)nc4c3F)c12]

To a solution of tert-butyl 6-hydroxy-1,4-oxazepane-4-carboxylate (803 mg, 3.70 mmol) in DCM (15 mL) was slowly added DAST (1.20 g, 7.44 mmol) at −50° C. The reaction solution was stirred at room temperature for 22 h. Then the resulting solution was quenched by saturated NaHCO₃ aqueous solution (30 mL) and extracted with DCM (40 mL, 20 mL). The organic layer was dried over anhydrous Na₂SO₄ and concentrated under vacuum. The residue was purified by silica gel chromatography (eluting with Hex:EA=15:1˜5:1) to give Compound 12-1 (683 mg, 3.12 mmol). MS m/z: 220 [M+H]⁺.

To a solution of Compound 12-1 (292 mg, 1.33 mmol) in acetone (3 mL) was added HCl (1 mL, 4M in 1,4-dioxane). The reaction solution was stirred at room temperature for 1 h. The solution was concentrated under vacuum. To the residue in DCM (15 mL) was added DIEA (669 mg, 5.18 mmol) and INT 1 (349 mg, 1.38 mmol). The reaction solution was stirred at room temperature for 3 h. The solution was concentrated under vacuum. The residue was dissolved in EA (50 mL), washed with brine (50 mL), dried over anhydrous Na₂SO₄ and concentrated in vacuum. The residue was washed with Hex:EA=10:1 (20 mL), filtered and the filtrate was concentrated under vacuum to give Compound 12-2 (405 mg, 1.21 mmol). MS m/z: 335 [M+H]⁺.

A solution of Compound 12-2 (405 mg, 1.21 mmol), INT 2 (286 mg, 1.80 mmol) and KF (204 mg, 3.51 mmol) in DMSO (15 mL) was stirred at 95° C. for 19 h under nitrogen atmosphere. The mixture was allowed to cool to room temperature, diluted with water (40 mL) and extracted with EA (2×30 mL). The organic layer was washed with NaCl aqueous solution (40 mL), then dried over anhydrous Na₂SO₄ and concentrated in vacuum. The residue was purified by Pre-TLC to give Compound 12-3 (254 mg, 0.55 mmol). MS m/z: 458 [M+H]⁺.

To a solution of Compound 12-3 (123 mg, 0.27 mmol), INT 3 (200 mg, 0.39 mmol) in toluene (4 mL) and water (1 mL) was added Cs₂CO₃ (187 mg, 0.57 mmol) and cataCXium A Pd G3 (27 mg, 0.037 mmol). The reaction mixture was stirred at 100° C. for 21 hours under nitrogen atmosphere. The reaction was diluted with EA (40 mL) and washed with brine (40 mL). The organic layer was dried over anhydrous Na₂SO₄ and concentrated under vacuum. The residue was purified by Pre-TLC to give Compound 12-4 (131 mg, 0.16 mmol). MS m/z: 808 [M+H]⁺.

To a solution of Compound 12-4 (131 mg, 0.16 mmol) in ACN (3 mL) was added HCl (1 mL, 4 mol/L in 1,4-dioxane) and stirred at room temperature for 1 h. The residue was diluted with saturated NaHCO₃ aqueous solution (20 mL) and extracted with EA (2×20 mL). The organic layer was dried over anhydrous Na₂SO₄ and concentrated under vacuum to give Compound 12-5 (103 mg, 0.13 mmol). MS m/z: 764 [M+H]⁺

To a solution of Compound 12-5 (103 mg, 0.13 mmol) in DMF (4 mL) was added CsF (0.34 g, 2.24 mmol). The reaction mixture was stirred at 40° C. overnight. The mixture was diluted with saturated NaHCO₃ aqueous solution (20 mL) and extracted with EA (2×20 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 35 min at a flow rate of 60 mL/min, 230 nm) to give Compound 12 (TFA salt). The product was separated by Prep-HPLC-Gilson with the following conditions: Column, CHIRAL ART Cellulose-SA column (2 cm×25 cm, 5 um); Mobile phase, (Hex:DCM=3:1) (0.1% isopropylamine)/MeOH(93:7); Flowing rate: 20 ml/min. This results in Compound 12A (13.4 mg, 0.022 mmol, the first eluting isomer, Retention Time 7.421 min) and Compound 12B (18.9 mg, 0.031 mmol, the second eluting isomer, Retention Time 8.085 min). MS m/z: 608 [M+H]⁺.

### Example 13

### 5-ethynyl-6-fluoro-4-(8-fluoro-2-(((2R,7aS)-2-fluorotetrahydro-1H-pyrrolizin-7a(5H)-yl)methoxy)-4-(6-methyl-1,4-oxazepan-4-yl)pyrido[4,3-d]pyrimidin-7-yl)naphthalen-2-ol trifluoroacetic acid (“Compound 13”)

### 5-ethynyl-6-fluoro-4-(8-fluoro-2-(((2R,7aS)-2-fluorotetrahydro-1H-pyrrolizin-7a(5H)-yl)methoxy)-4-((S)-6-methyl-1,4-oxazepan-4-yl)pyrido[4,3-d]pyrimidin-7-yl)naphthalen-2-ol (“Compound 13A” or “Compound 13B”)

### 5-ethynyl-6-fluoro-4-(8-fluoro-2-(((2R,7aS)-2-fluorotetrahydro-1H-pyrrolizin-7a(5H)-yl)methoxy)-4-((R)-6-methyl-1,4-oxazepan-4-yl)pyrido[4,3-d]pyrimidin-7-yl)naphthalen-2-ol (“Compound 13B or Compound 13A”)

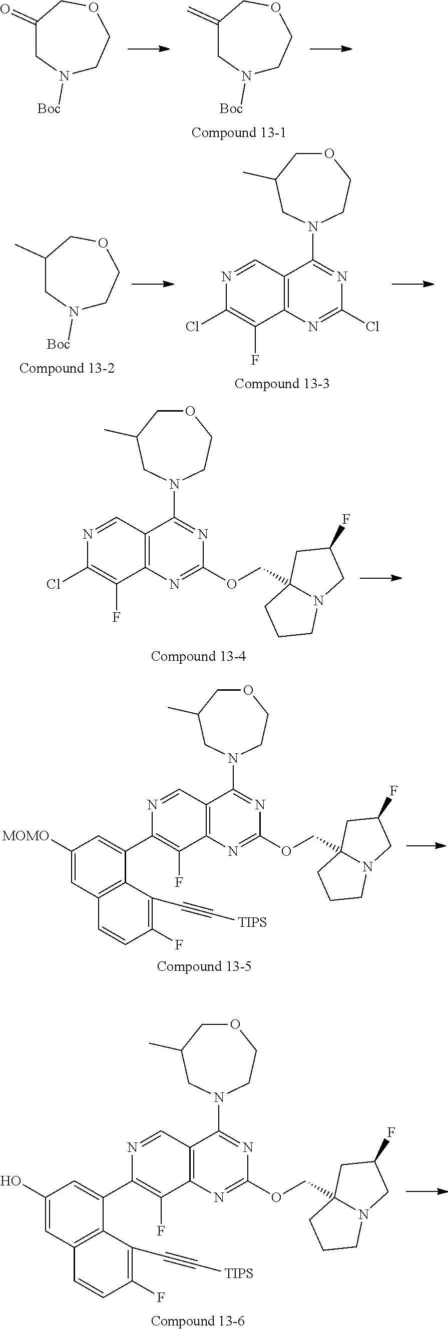

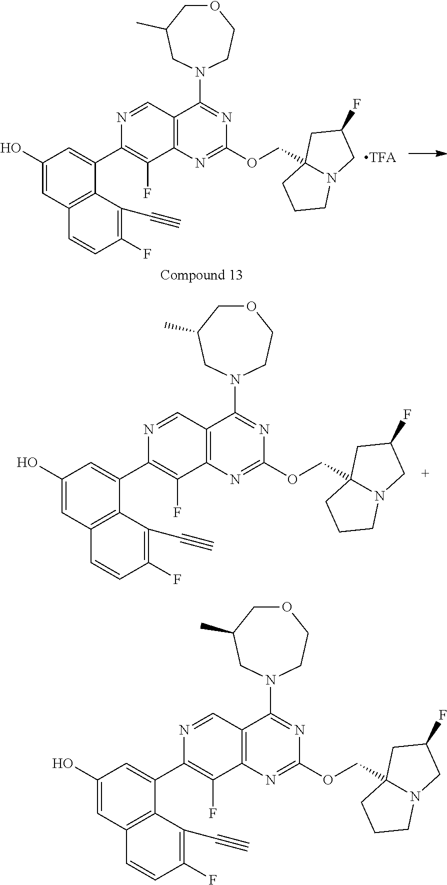

To a solution of methyltriphenylphosphonium bromide (746 mg, 2.09 mmol) in THF (10 mL) was added t-BuOK (235 mg, 2.09 mmol) at 0° C. The reaction solution was stirred at 0° C. for 0.5 h. To the solution was added tert-butyl 6-oxo-1,4-oxazepane-4-carboxylate (301 mg, 1.40 mmol) in THF (1 mL). The reaction solution was stirred at room temperature for 3 h. The solution was filtered and the filtrate was concentrated under vacuum. The residue was purified by silica gel chromatography (eluting with Hex:EA=15:1˜8:1) to give Compound 13-1 (288 mg, 1.35 mmol). MS m/z: 114 [M+H−100]⁺.

To a solution of Compound 13-1 (288 mg, 1.35 mmol) in MeOH (15 mL) was added Pd(OH)₂/C (135 mg, 25% Pd content). The reaction solution was stirred at 25° C. for 2 h under hydrogen atmosphere. The solution was filtered and the filtrate was concentrated under vacuum to give Compound 13-2 (227 mg, 1.05 mmol). MS m/z: 116 [M+H−100]⁺.

To a solution of Compound 13-2 (227 mg, 1.05 mmol) in acetone (4.5 mL) was added HCl (1.5 mL, 4 mol/L in 1,4-dioxane). The reaction solution was stirred at room temperature for 1.5 h. The solution was concentrated under vacuum. To the residue in DCM (15 mL) was added DIEA (491 mg, 3.80 mmol) and INT 1 (269 mg, 1.07 mmol). The reaction solution was stirred at room temperature for 1 h. The solution was diluted with water (30 mL) and extracted with DCM (2×30 mL). The organic layer was washed with NaCl aqueous solution (40 mL), then dried over anhydrous Na₂SO₄ and concentrated in vacuum. The residue was purified by Pre-TLC to give Compound 13-3 (279 mg, 0.84 mmol). MS m/z: 331 [M+H]⁺.

A solution of Compound 13-3 (279 mg, 0.84 mmol), INT 2 (229 mg, 1.44 mmol) and KF (154 mg, 2.65 mmol) in DMSO (10 mL) was stirred at 95° C. for 16 h under nitrogen atmosphere. The mixture was allowed to cool to room temperature, diluted with water (40 mL) and extracted with EA (2×30 mL). The organic layer was washed with NaCl aqueous solution (40 mL), then dried over anhydrous Na₂SO₄ and concentrated in vacuum. The residue was purified by Pre-TLC to give Compound 13-4 (416 mg, 0.92 mmol). MS m/z: 454 [M+H]⁺.

To a solution of Compound 13-4 (200 mg, 0.44 mmol), INT 3 (309 mg, 0.60 mmol) in toluene (6 mL) and water (1.5 mL) were added Cs₂CO₃ (312 mg, 0.96 mmol) and cataCXium A Pd G3 (45 mg, 0.062 mmol). The reaction mixture was stirred at 100° C. for 19 hours under nitrogen atmosphere. The reaction was diluted with EA (40 mL) and washed with brine (40 mL). The organic layer was dried over anhydrous Na₂SO₄ and concentrated under vacuum. The residue was purified by Pre-TLC to give Compound 13-5 (197 mg, 0.25 mmol). MS m/z: 804 [M+H]⁺.

To a solution of Compound 13-5 (197 mg, 0.25 mmol) in ACN (4.5 mL) was added HCl (1.5 mL, 4 mol/L in dioxane) and stirred at room temperature for 1 h. The residue was diluted with saturated NaHCO₃ aqueous solution (20 mL) and extracted with EA (2×20 mL). The organic layer was dried over anhydrous Na₂SO₄ and concentrated under vacuum to give Compound 13-6 (180 mg, 0.24 mmol). MS m/z: 760 [M+H]⁺.

To a solution of Compound 13-6 (180 mg, 0.24 mmol) in DMF (6 mL) was added CsF (0.83 g, 5.46 mmol). The reaction mixture was stirred at 40° C. overnight. The mixture was diluted with saturated NaHCO₃ aqueous solution (20 mL) and extracted with EA (2×20 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 33% B in 35 min at a flow rate of 60 mL/min, 235 nm) to give Compound 13 (TFA salt). The product was separated by Prep-HPLC-Gilson with the following conditions: Column, CHIRAL ART Cellulose-SA column (2 cm×25 cm, 5 um); Mobile phase, (Hex:DCM=3:1) (0.1% isopropylamine)/MeOH (91:9); Flowing rate: 20 ml/min. This results in Compound 13A (32.3 mg, 0.053 mmol, the first eluting isomer, Retention Time 5.202 min) and Compound 13B (33.1 mg, 0.055 mmol, the second eluting isomer, Retention Time 5.743 min) MS m/z: 604 [M+H]⁺.

### Example 14

### 2-(4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)acetonitrile trifluoroacetic acid (“Compound 14”)

### 2-((S)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)acetonitrile

### 2-((R)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)acetonitrile

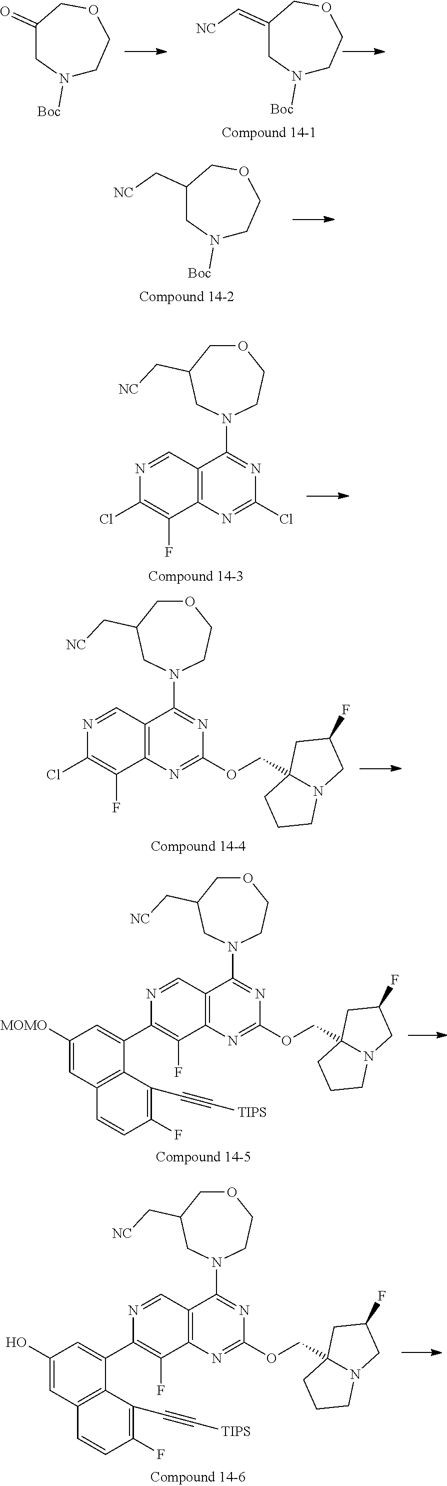

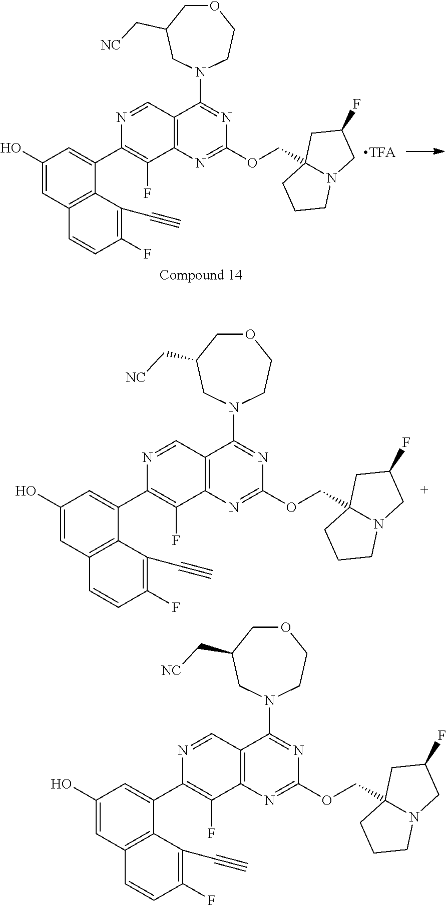

To a solution of diethyl cyanomethylphosphonate (306 mg, 1.73 mmol) in THF (10 mL) was added NaH (71 mg, 1.78 mmol, 60% content) at 0° C. The reaction solution was stirred at 0° C. for 0.5 h. To the solution was added tert-butyl 6-oxo-1,4-oxazepane-4-carboxylate (306 mg, 1.42 mmol) in THF (2 mL). The reaction solution was stirred at room temperature for 1.5 h. The solution was diluted with water (30 mL) and extracted with EA (2×30 mL). The organic layer was washed with NaCl aqueous solution (40 mL), then dried over anhydrous Na₂SO₄ and concentrated in vacuum. The residue was purified by silica gel chromatography to give Compound 14-1 (305 mg, 1.28 mmol). MS m/z: 139 [M+H−100]*.

To a solution of Compound 14-1 (305 mg, 1.28 mmol) in MeOH (15 mL) was added Pd/C (282 mg, 10% Pd content). The reaction solution was stirred at 25° C. for 1.5 h under hydrogen atmosphere. The solution was filtered and the filtrate was concentrated under vacuum to give Compound 14-2 (286 mg, 1.19 mmol). MS m/z: 141 [M+H−100]⁺.

To a solution of Compound 14-2 (227 mg, 1.05 mmol) in acetone (6 mL) was added HCl (2 mL, 4 mol/L in 1,4-dioxane). The reaction solution was stirred at room temperature for 1 h. The solution was concentrated under vacuum. To the residue in DCM (10 mL) was added DIEA (541 mg, 4.19 mmol) and INT 3 (335 mg, 1.33 mmol). The reaction solution was stirred at room temperature for 0.5 h. The solution was diluted with water (20 mL) and extracted with DCM (2×20 mL). The organic layer was washed with NaCl aqueous solution (40 mL), then dried over anhydrous Na₂SO₄ and concentrated in vacuum. The residue was purified by Pre-TLC to give Compound 14-3 (356 mg, 1.00 mmol). MS m/z: 356 [M+H]⁺.

A solution of Compound 14-3 (356 mg, 1.00 mmol), INT 2 (254 mg, 1.60 mmol) and KF (195 mg, 3.36 mmol) in DMSO (10 mL) was stirred at 95° C. for 18 h under nitrogen atmosphere. The mixture was allowed to cool to room temperature, diluted with water (40 mL) and extracted with EA (2×30 mL). The organic layer was washed with NaCl aqueous solution (40 mL), then dried over anhydrous Na₂SO₄ and concentrated in vacuum. The residue was purified by Pre-TLC to give Compound 14-4 (286 mg, 0.60 mmol). MS m/z: 479 [M+H]⁺.

To a solution of Compound 14-4 (153 mg, 0.32 mmol), INT 3 (212 mg, 0.41 mmol) in toluene (6 mL) and water (1.5 mL) were added Cs₂CO₃ (228 mg, 0.70 mmol) and cataCXium A Pd G3 (34 mg, 0.047 mmol). The reaction mixture was stirred at 100° C. for 19 hours under nitrogen atmosphere. The reaction was diluted with EA (40 mL) and washed with brine (40 mL). The organic layer was dried over anhydrous Na₂SO₄ and concentrated under vacuum. The residue was purified by Pre-TLC to give Compound 14-5 (182 mg, 0.22 mmol). MS m/z: 829 [M+H]⁺.

To a solution of Compound 14-5 (182 mg, 0.22 mmol) in ACN (4.5 mL) was added HCl (1.5 mL, 4 mol/L in dioxane) and stirred at room temperature for 1 h. The residue was diluted with saturated NaHCO₃ aqueous solution (20 mL) and extracted with EA (2×20 mL). The organic layer was dried over anhydrous Na₂SO₄ and concentrated under vacuum to give Compound 14-6 (167 mg, 0.21 mmol). MS m/z: 785 [M+H]⁺.

To a solution of Compound 14-6 (167 mg, 0.21 mmol) in DMF (5 mL) was added CsF (0.59 g, 3.88 mmol). The reaction mixture was stirred at 40° C. overnight. The mixture was diluted with saturated NaHCO₃ aqueous solution (20 mL) and extracted with EA (2×20 mL). The organic layers were combined, dried over anhydrous Na₂SO₄ and concentrated under vacuum. The residue was purified by Prep-HPLC (C18 column, A: 0.1% TFA in water, B: CH₃CN, Gradient: 10% B to 40% B in 60 min at a flow rate of 70 mL/min, 237 nm) to give Compound 14 (TFA salt). The product was separated by Prep-HPLC-Gilson with the following conditions: Column, CHIRAL ART Cellulose-SA column (2 cm×25 cm, 5 um); Mobile phase, (Hex:DCM=3: 1) (0.1% isopropylamine)/MeOH(93:7); Flowing rate: 20 ml/min. This results in Compound 14A (32.2 mg, 0.051 mmol, the first eluting isomer, Retention Time 6.792 min) and Compound 14B (35.2 mg, 0.056 mmol, the second eluting isomer, Retention Time 7.476 min) MS m/z: 629 [M+H]⁺.

### Example 15

### 2-(4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-2-yl)acetonitrile trifluoroacetic acid (“Compound 15”)

### 2-((R)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-2-yl)acetonitrile (“Compound 15A” or “Compound 15B”)

### 2-((S)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-2-yl)acetonitrile (“Compound 15B” or “Compound 15A”)

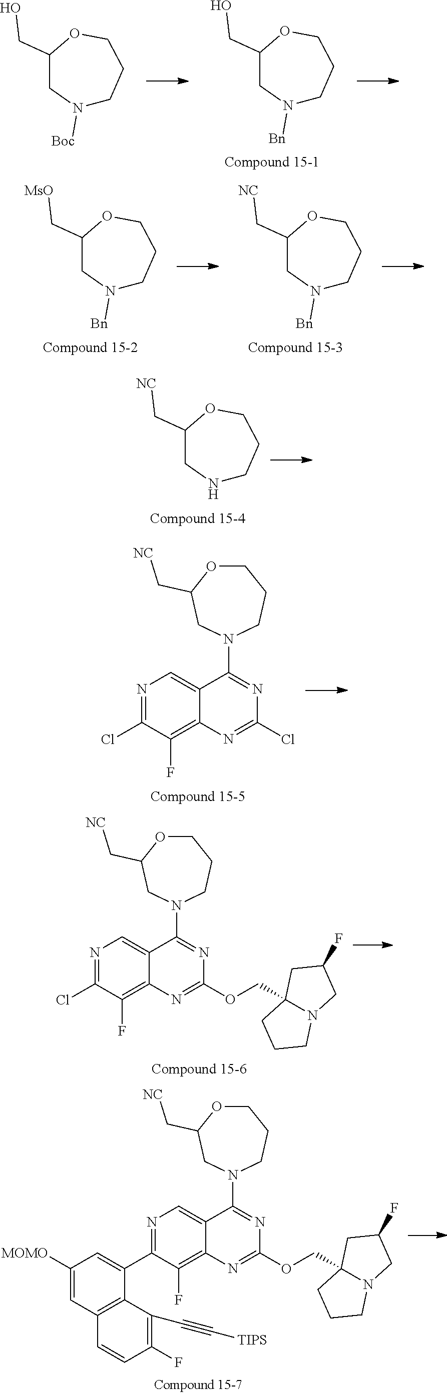

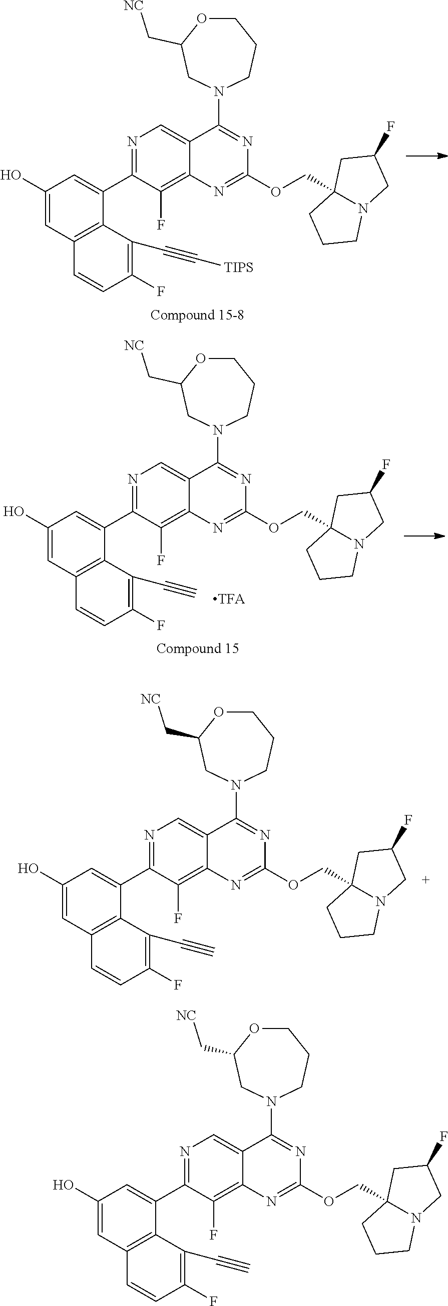

To a solution of tert-butyl 2-(hydroxymethyl)-1,4-oxazepane-4-carboxylate (197 mg, 0.85 mmol) in acetone (4.5 mL) was added HCl (1.5 mL, 4 mol/L in 1,4-dioxane). The reaction solution was stirred at room temperature for 2 h. The solution was concentrated under vacuum. To the residue in DCM (10 mL) was added DIEA (356 mg, 2.75 mmol) and (bromomethyl)benzene (167 mg, 0.98 mmol). The reaction solution was stirred at room temperature for 2.5 h. The solution was diluted with water (30 mL) and extracted with EA (2×30 mL). The organic layer was washed with aqueous NaCl solution (40 mL), then dried over anhydrous Na₂SO₄ and concentrated in vacuum. The residue was purified by silica gel chromatography (eluting with DCM:MeOH=50:1˜30:1) to give Compound 15-1 (136 mg, 0.61 mmol). MS m/z: 222 [M+H]⁺.

To a solution of Compound 15-1 (136 mg, 0.61 mmol) in THF (10 mL) was added TEA (193 mg, 1.91 mmol) and methanesulfonyl chloride (114 mg, 1.00 mmol) at 0° C. The reaction solution was stirred at 0° C. for 15 min. The solution was diluted with water (30 mL) and extracted with EA (2×30 mL). The organic layer was washed with NaCl aqueous solution (40 mL), then dried over anhydrous Na₂SO₄ and concentrated in vacuum to give Compound 15-2 (200 mg, 0.67 mmol). MS m/z: 300 [M+H]⁺.

To a solution of Compound 15-2 (200 mg, 0.67 mmol) in DMF (10 mL) was added CsF (516 mg, 3.40 mmol) and cyanotrimethylsilaneat (306 mg, 3.08 mmol). The reaction solution was stirred at 80° C. for 5 h. The solution was diluted with water (30 mL) and extracted with EA (2×30 mL). The organic layer was washed with NaCl aqueous solution (40 mL) then dried over anhydrous Na₂SO₄ and concentrated in vacuum. The residue was purified by silica gel chromatography (eluting with Hex:EA=10:1˜3:1) to give Compound 15-3 (120 mg, 0.52 mmol). MS m/z: 231 [M+H]⁺.

To a solution of Compound 15-3 (292 mg, 1.33 mmol) in ethylene chloride (10 mL) was added 1-chloroethyl carbonochloridate (238 mg, 1.66 mmol). The reaction solution was stirred at room temperature for 2 h. The solution was concentrated under vacuum. To the residue was added MeOH (10 mL). The reaction solution was stirred at 60° C. for 1.5 h. The solution was concentrated under vacuum. The residue was purified by silica gel chromatography (eluting with Hex:EA=10:1-DCM:MeOH=8:1) to give Compound 15-4 (101 mg, 0.72 mmol). MS m/z: 141 [M+H]⁺.

To a solution of Compound 15-4 in DCM (10 mL) was added DIEA (240 mg, 1.86 mmol) and INT 1 (129 mg, 0.51 mmol). The reaction solution was stirred at room temperature for 1 h. The solution was diluted with water (30 mL) and extracted with DCM (2×30 mL). The organic layer was washed with NaCl aqueous solution (40 mL), then dried over anhydrous Na₂SO₄ and concentrated in vacuum. The residue was purified by Pre-TLC to give Compound 15-5 (117 mg, 0.33 mmol). MS m/z: 356 [M+H]⁺.

A solution of Compound 15-5 (117 mg, 0.33 mmol), INT 2 (84 mg, 0.53 mmol) and KF (57 mg, 0.98 mmol) in DMSO (5 mL) was stirred at 95° C. for 2 h under nitrogen atmosphere. The mixture was allowed to cool to room temperature, diluted with water (40 mL) and extracted with EA (2×30 mL). The organic layer was washed with NaCl aqueous solution (40 mL) then dried over anhydrous Na₂SO₄ and concentrated in vacuum. The residue was purified by Pre-TLC to give Compound 15-6 (54 mg, 0.11 mmol). MS m/z: 479 [M+H]⁺.

To a solution of Compound 15-6 (54 mg, 0.11 mmol), INT 3 (94 mg, 0.18 mmol) in toluene (4 mL) and water (1 mL) were added Cs₂CO₃ (89 mg, 0.27 mmol) and cataCXium A Pd G3 (24 mg, 0.033 mmol). The reaction mixture was stirred at 100° C. for 20 hours under nitrogen atmosphere. The reaction was diluted with EA (30 mL) and washed with brine (40 mL). The organic layer was dried over anhydrous Na₂SO₄ and concentrated under vacuum. The residue was purified by Pre-TLC to give Compound 15-7 (64 mg, 0.077 mmol). MS m/z: 829 [M+H]⁺.

To a solution of Compound 15-7 (64 mg, 0.077 mmol) in ACN (3 mL) was added HCl (1 mL, 4 mol/L in 1,4-dioxane) and stirred at room temperature for 1 h. The residue was diluted with saturated NaHCO₃ aqueous solution (20 mL) and extracted with EA (2×20 mL). The organic layers were combined, dried over anhydrous Na₂SO₄ and concentrated under vacuum to give Compound 15-8 (62 mg, 0.079 mmol). MS m/z: 785 [M+H]⁺.

To a solution of Compound 15-8 (62 mg, 0.079 mmol) in DMF (4 mL) was added CsF (0.48 g, 3.16 mmol). The reaction mixture was stirred at 40° C. overnight. The mixture was diluted with saturated NaHCO₃ aqueous solution (20 mL) and extracted with EA (2×20 mL). The organic layers were combined, dried over anhydrous Na₂SO₄ and concentrated under vacuum. The residue was purified by Prep-HPLC (C18 column, A: 0.1% TFA in water, B: CH₃CN, Gradient: 30% B to 60% B in 60 min at a flow rate of 40 mL/min, 236 nm) to give Compound 15 (TFA salt). The Compound 15 was separated by Prep-HPLC-Gilson with the following conditions: Column, CHIRAL ART Cellulose-SC column (2 cm×25 cm, 5 m); Mobile phase, (Hex:DCM=3:1)(0.1% isopropylamine)/EtOH(75:25); Flowing rate: 20 ml/min. This results in Compound 15A (8.2 mg, 0.013 mmol, the first eluting isomer, Retention Time 6.535 min) and Compound 15B (9.6 mg, 0.015 mmol, the second eluting isomer, Retention Time 7.594 min). MS m/z: 629 [M+H]⁺.

### Example 16

### 5-ethynyl-6-fluoro-4-(8-fluoro-2-(((2R,7aS)-2-fluorotetrahydro-1H-pyrrolizin-7a(5H)-yl)methoxy)-4-(3-methyl-1,4-oxazepan-4-yl)pyrido[4,3-d]pyrimidin-7-yl)naphthalen-2-ol trifluoroacetic acid (“Compound 16”)

### 5-ethynyl-6-fluoro-4-(8-fluoro-2-(((2R,7aS)-2-fluorotetrahydro-1H-pyrrolizin-7a(5H)-yl)methoxy)-4-((S)-3-methyl-1,4-oxazepan-4-yl)pyrido[4,3-d]pyrimidin-7-yl)naphthalen-2-ol

### 5-ethynyl-6-fluoro-4-(8-fluoro-2-(((2R,7aS)-2-fluorotetrahydro-1H-pyrrolizin-7a(5H)-yl)methoxy)-4-((R)-3-methyl-1,4-oxazepan-4-yl)pyrido[4,3-d]pyrimidin-7-yl)naphthalen-2-ol

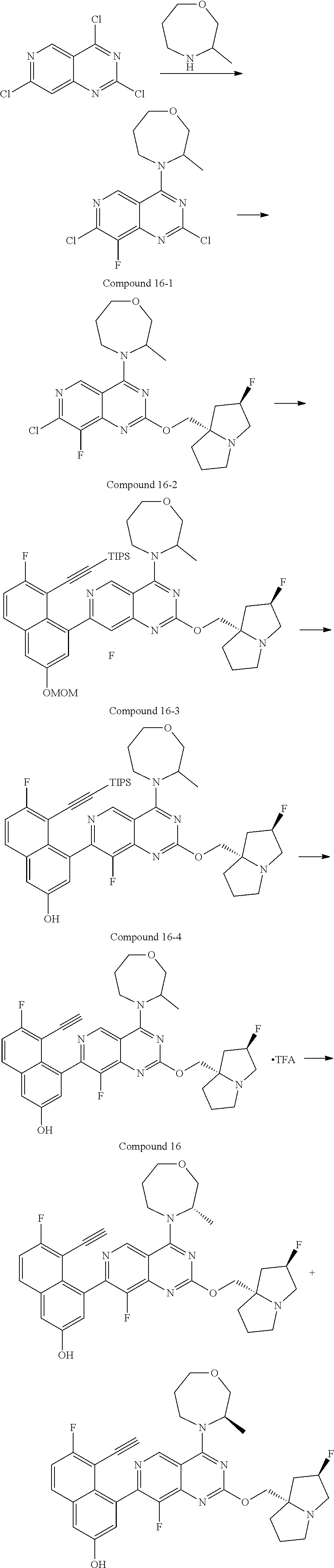

To a solution of INT 1 (335 mg, 1.3269 mmol) and DIEA (0.3 mL) in DCM (10.0 mL) was added 3-methyl-1,4-oxazepane (119 mg, 1.0332 mmol) at 0° C. The reaction mixture was stirred for 1 hour at room temperature. Then the mixture was concentrated in vacuum, diluted with EA (50 mL) and washed with water (2×30 mL) and brine (30 mL). The organic layer was dried over anhydrous Na₂SO₄ and concentrated in vacuum to give Compound 16-1 (415 mg, 1.2531 mmol, 94.4369% yield). MS (ESI, m/z): 331 [M+H]⁺.

To a solution of Compound 16-1 (415 mg, 1.2531 mmol) and INT 2 (400 mg, 2.5125 mmol) in DMSO (8 mL) was added KF (232 mg, 3.9933 mmol) at room temperature. The reaction mixture was stirred at 100° C. for 16 hours. The reaction mixture was quenched by water (100 mL) and extracted with EA (2×50 mL). The organic layer was washed with brine (50 mL), dried over anhydrous Na₂SO₄ and concentrated in vacuum. The residue was purified by Pre-TLC (DCM:MeOH=15:1, v/v) to give Compound 16-2 (414 mg, 912.0707 μmol, 72.7837% yield). MS (ESI, m/z): 454 [M+H]⁺.

A solution of Compound 16-2 (71 mg, 156.4179 μmol), INT 3 (121 mg, 236.0816 μmol), cataCXium A Pd G3 (19 mg, 26.0892 μmol), Cs₂CO₃ (183 mg, 561.6617 μmol) in toluene (4 mL) and water (1 mL) was stirred at 100° C. for 16 hours under nitrogen atmosphere. The reaction was diluted with EA (50 mL) and washed with water (3×30 mL). The organic layer was dried over anhydrous Na₂SO₄ and concentrated under vacuum. The residue was purified by Pre-TLC (DCM:MeOH=15:1, v/v) to give Compound 16-3 (80 mg, 99.4994 μmol, 63.6113% yield). MS (ESI, m/z): 804 [M+H]⁺.

To a solution of Compound 16-3 (80 mg, 99.4994 μmol) in CH₃CN (6 mL) was added HCl (4 M in 1,4-dioxane, 2 mL). The reaction mixture was stirred at room temperature for 1 hour. The resulting mixture was concentrated under reduced pressure. The residue was dissolved in EA (50 mL) and washed with saturated NaHCO₃ aqueous solution (3×30 mL). The organic layer was dried over anhydrous Na₂SO₄ and concentrated under reduced pressure to give Compound 16-4 (71 mg, 93.4244 μmol, 93.8945% yield). MS (ESI, m/z): 760 [M+H]⁺.

To a solution of Compound 16-4 (71 mg, 93.4244 μmol) in DMF (3 mL) was added CsF (0.37 g, 2.4358 mmol) and the reaction mixture was stirred at 40° C. for 16 hours. The resulting mixture was concentrated under reduced pressure and the residue was purified by Pre-HPLC (YMC-Triart C18-S12 nm column, 50×250 mm, 10 μm, A: 0.1% TFA in water, B: CH₃CN, Gradient: 15% B to 60% B in 35 min at a flow rate of 70 mL/min, 240 nm) to give Compound 16 (36.5 mg, 50.8600 μmol, 54.4398% yield, TFA salt). MS (ESI, m/z): 604 [M+H]⁺.

Compound 16 (30 mg, 41.8028 μmol) was separated by Prep-HPLC-Gilson with the following conditions: Column, CHIRAL ART Cellulose-SC column (2 cm×25 cm, 5 μm); Mobile phase, Hex (0.1% isopropylamine)/EtOH (60:40); Flowing rate: 20 mL/min to give Compound 16A (8 mg, the first eluting isomer, Retention Time 6.643 min) and Compound 16B (7 mg, the second eluting isomer, Retention Time 8.188 min) respectively.

### Example 17

### 5-ethynyl-6-fluoro-4-(8-fluoro-2-(((2R,7aS)-2-fluorotetrahydro-1H-pyrrolizin-7a(5H)-yl)methoxy)-4-(7-methyl-1,4-oxazepan-4-yl)pyrido[4,3-d]pyrimidin-7-yl)naphthalen-2-ol trifluoroacetic acid (“Compound 17”)

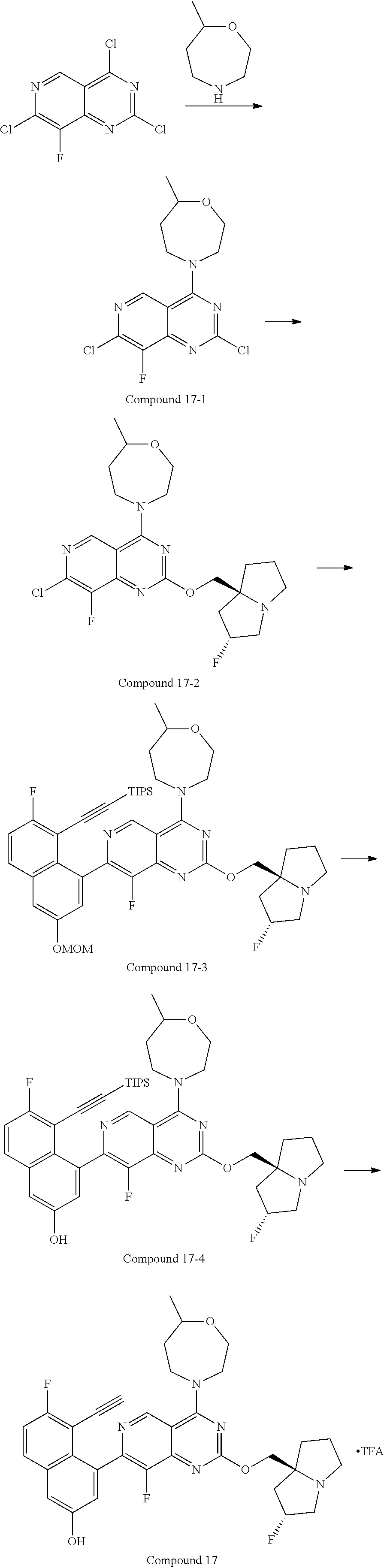

A solution of INT 1 (350 mg, 1.39 mmol), 7-methyl-1,4-oxazepane hydrochloride (199 mg, 1.31 mmol) and DIEA (551 mg, 4.26 mmol) in DCM (10 mL) was stirred at 0° C. for 1 h. The solution was diluted with water (30 mL) and extracted with DCM (40 mL). The organic layer was washed with brine, dried over anhydrous Na₂SO₄ and concentrated in vacuum. The residue was purified by Pre-TLC to give Compound 17-1 (421 mg, 1.27 mmol, 91.70% yield). MS: m/z 331 [M+H]⁺.

A solution of Compound 17-1 (421 mg, 1.27 mmol), INT 2 (302 mg, 1.90 mmol) and KF (296 mg, 5.09 mmol) in DMSO (8 mL) was stirred at 85° C. for 20 h under nitrogen atmosphere. The mixture was diluted with EA (40 mL) and water (30 mL). The organic layer was washed with brine (30 mL), dried over anhydrous Na₂SO₄, filtered and concentrated under reduced pressure. The residue was purified by Pre-TLC (DCM:MeOH=15:1, v/v) to give Compound 17-2 (434 mg, 0.96 mmol, 75.60% yield). MS: m/z 454 [M+H]⁺.

A solution of Compound 17-2 (96 mg, 0.21 mmol), toluene (5 mL), INT 3 (174 mg, 0.34 mmol), cataCXium A Pd G3 (15 mg, 20.60 μmol), Cs₂CO₃ (205 mg, 0.63 mmol) and water (1 mL) was stirred at 105° C. for 16 hours under nitrogen atmosphere. The mixture was allowed to cool to room temperature and diluted with water (30 mL) and extracted with EA (40 mL). The organic layer was washed with brine then dried over anhydrous Na₂SO₄ and concentrated in vacuum. The residue was purified by Pre-TLC (DCM:MeOH=15:1, v/v) to give Compound 17-3 (107 mg, 131.44 μmol, 62.59% yield). MS: m/z 804 [M+H]⁺.

To a solution of Compound 17-3 (107 mg, 131.44 μmol) in CH₃CN (5 mL) was added HCl (4 M in 1,4-dioxane, 2 mL). The reaction mixture was stirred at room temperature for 1 h. After completion, the reaction mixture was concentrated under reduced pressure. The residue was diluted with EA (40 mL) and water (30 mL) and the mixture was adjusted to pH 8-9 with saturated NaHCO₃ aqueous solution. The organic layer was separated and concentrated under reduced pressure to give Compound 17-4 (104 mg, crude). MS: m/z 760 [M+H]⁺.

To a mixture of Compound 17-4 (104 mg, crude) in DMF (5 mL) was added CsF (242 mg, 1.59 mmol). The mixture was stirred at room temperature for 20 hours. After completion, the mixture was diluted with EA (40 mL) and water (30 mL) and the mixture was adjusted to pH 8-9 with saturated NaHCO₃ aqueous solution. The organic layer was washed with brine, dried over anhydrous Na₂SO₄ and concentrated in vacuum. The residue was purified by Prep-HPLC (C18 column, A: 0.1% TFA in water, B: CH₃CN, Gradient: 12% B to 40% B in 60 min at a flow rate of 70 mL/min, 240 nm) to give Compound 17 (68.4 mg, 95.31 μmol, TFA salt). MS: m/z 604 [M+H]⁺.

### Example 18

### 4-(4-(6,6-difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol trifluoroacetic acid (“Compound 18”)

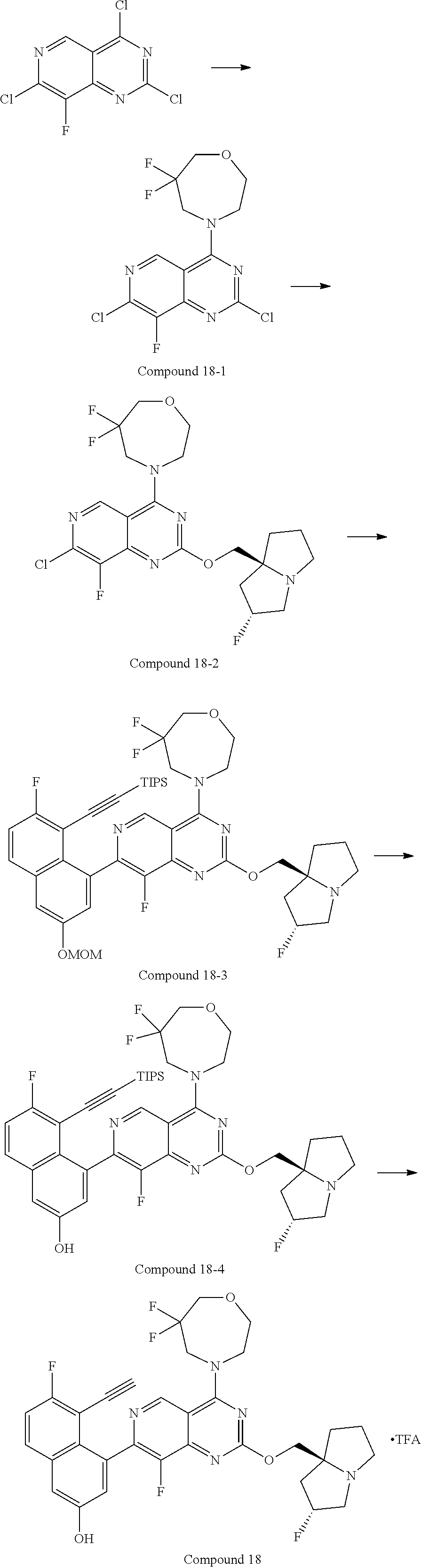

A solution of INT 1 (204 mg, 0.81 mmol), 6,6-difluoro-1,4-oxazepane hydrochloride (116 mg, 0.67 mmol) and DIEA (310 mg, 2.40 mmol) in DCM (10 mL) was stirred at 0° C. for 1 h. The solution was diluted with water (30 mL) and extracted with DCM (40 mL). The organic layer was washed with 5b citric acid (30 mL), brine (30 mL), dried over anhydrous Na₂SO₄ and concentrated in vacuum to give Compound 18-1 (270 mg, 0.76 mmol, 94.62% yield). MS: m/z 353 [M+H]⁺.

A solution of Compound 18-1 (270 mg, 0.76 mmol), NT 2 (183 mg, 1.15 mmol) and KF (180 mg, 3.10 mmol) in DMSO (8 mL) was stirred at 85° C. for 18 h under nitrogen atmosphere. The mixture was diluted with EA (40 mL) and water (40 mL). The organic layer was washed with brine (40 mL), dried over anhydrous Na₂SO₄, filtered and concentrated under reduced pressure. The residue was purified by Pre-TLC (DCM:MeOH=20:1, v/v) to give Compound 18-2 (245 mg, 0.51 mmol, 67.34% yield). MS: m/z 476 [M+H]⁺.

A solution of Compound 18-2 (115 mg, 0.24 mmol), toluene (8 mL), CNT 3 (204 mg, 0.40 mmol), cataCXium A Pd G3 (19 mg, 26.09 μmol), Cs₂CO₃ (205 mg, 0.63 mmol) and water (2 mL) was stirred at 105° C. for 17 hours under nitrogen atmosphere. The mixture was allowed to cool to room temperature, diluted with water (30 mL) and extracted with EA (40 mL). The organic layer was washed with brine then dried over anhydrous Na₂SO₄ and concentrated in vacuum. The residue was purified by Pre-TLC (DCM:MecOH=15:1, v/v) to give Compound 18-3 (140 mg, 169.50 μmol, 70.14% yield). MS: m/z 826 [M+H]⁺.

To a solution of Compound 18-3 (140 mg, 169.50 μmol) in CH₃CN (5 mL) was added HCl (4 M in 1,4-dioxane, 2 mL). The reaction mixture was stirred at room temperature for 1 h. After completion, the reaction mixture was concentrated under reduced pressure. The residue was diluted with EA (40 mL) and water (30 mL) and the mixture was adjusted to pH 8-9 with saturated NaHCO₃ aqueous solution. The organic layer was separated and concentrated under reduced pressure to give Compound 18-4 (143 mg, crude). MS: m/z 782 [M+H]⁺.

To a mixture of Compound 18-4 (140 mg) in DMF (5 mL) was added CsF (273 mg, 1.80 mmol). The mixture was stirred at room temperature for 20 hours. After completion, the mixture was diluted with EA (40 mL) and water (30 mL), and the mixture was adjusted to pH 8-9 with saturated NaHCO₃ aqueous solution. The organic layer was washed with brine then dried over anhydrous Na₂SO₄ and concentrated in vacuum. The residue was purified by Prep-HPLC (C18 column, A: 0.1% TFA in water, B: CH₃CN, Gradient: 15% B to 45% B in 45 min at a flow rate of 60 mL/min, 240 nm) to give Compound 18 (85.1 mg, 115.06 μmol, TFA salt). MS: m/z 626 [M+H]⁺.

### Example 19

### 4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-5-ethynyl-6-fluoronaphthalen-2-ol trifluoroacetic acid (“Compound 19”)

### (P)-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-5-ethynyl-6-fluoronaphthalen-2-ol trifluoroacetic acid (“Compound 19A or Compound 19B”)

### (M)-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-5-ethynyl-6-fluoronaphthalen-2-ol trifluoroacetic acid (“Compound 19B or Compound 19A”)

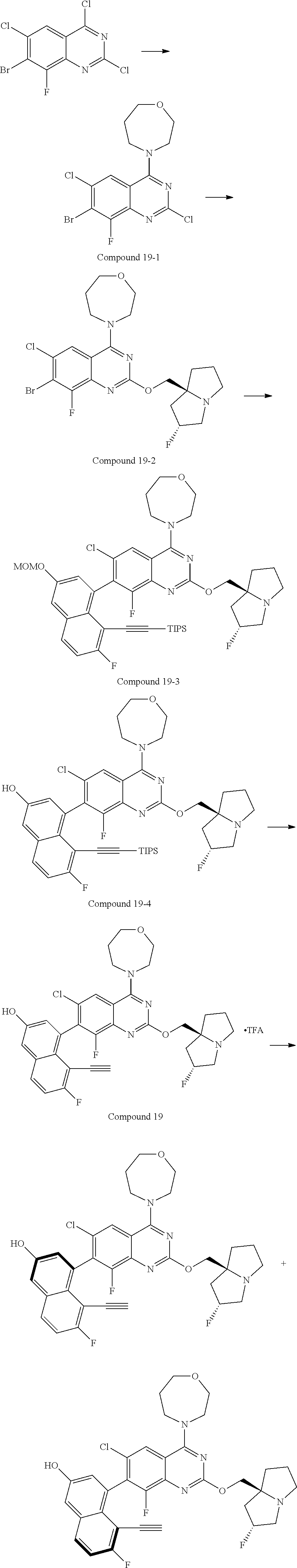

A solution of 7-bromo-2,4,6-trichloro-8-fluoroquinazoline (199 mg, 602.3580 μmol), 1,4-oxazepane (66 mg, 652.5189 μmol) and DIEA (243 mg, 1.8802 mmol) in DCM (8 mL) was stirred 3 h at room temperature. The solution was diluted with saturated NaHCO₃ aqueous solution (10 mL). The organic layer was washed with saturated NaCl aqueous solution, dried over anhydrous Na₂SO₄ and concentrated in vacuum to give Compound 19-1 (292 mg, 739.1396 mol, 122.7077% yield). MS m/z: 394 [M+H]F.

A solution of Compound 19-1 (292 mg, 739.1396 μmol), INT 2 (103 mg, 646.9821 μmol), triethylene diamine (38 mg, 338.7655 μmol), cesium carbonate (270 mg, 828.6812 μmol) in THF (4 mL) and N,N-dimethylformamide (4 mL) was stirred at room temperature for 18 h under nitrogen atmosphere. The mixture was diluted with saturated NaHCO₃ aqueous solution (10 mL) and extracted with EA (10 mL). The organic layer was washed with 10 mL saturated NaCl aqueous solution, then dried over anhydrous Na₂SO₄ and concentrated in vacuum. The residue was purified by Pre-TLC to give Compound 19-2 (225 mg, 434.5360 mol, 58.7894% yield). MS m/z: 517 [M+H]⁺.

A solution of Compound 19-2 (0.225 g, 434.5357 mol), INT 3 (260 mg, 507.2828 mol), cataCXium A Pd G3 (35 mg, 48.0591 μmol), potassium phosphate (310 mg, 1.4604 mmol) in toluene (5 mL) and water (1 mL) was stirred at 100° C. for 16 hours under nitrogen atmosphere. The mixture was allowed to cool to room temperature, diluted with saturated NaHCO₃ aqueous solution (10 mL) and extracted with EA (10 mL). The organic layer was washed with 10 mL saturated NaCl aqueous solution, then dried over anhydrous Na₂SO₄ and concentrated in vacuum. The residue was purified by Pre-TLC to give Compound 19-3 (292 mg, 354.60331 mol, 81.6051% yield). MS m/z: 823 [M+H].

A solution of Compound 19-3 (292 mg, 354.6033 μmol) and HCl (4 M in dioxane, 1 mL) in DCM (10 mL) was stirred at room temperature for 2 h. The solution was diluted with DCM (20 mL) and 10% NaHCO₃ solution (20 mL). The organic layer was washed with saturated NaCl aqueous solution, dried over anhydrous Na₂SO₄ and concentrated in vacuum to give Compound 19-4 (302 mg, 387.4760 mol, 109.2704% yield). MS m/z: 779 [M+H]⁺.

A solution of Compound 19-4 (0.302 g, 387.4760 μmol) and CsF (340 mg, 2.2383 mmol) in DMF (8 mL) was stirred for 20 hours at 40° C. under nitrogen atmosphere. The solution was diluted with water (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 50% B in 47 min at a flow rate of 60 mL/min, 260 nm) and freeze-dried to give Compound 19 (105 mg, 168.5222 mol, 43.4923% yield, TFA salt). MS m/z: 622 [M+H]⁺. The Compound 19 was separated by Prep-HPLC-Gilson with the following conditions: Column, CHIRAL ART Cellulose-SA column (2 cm×25 cm, 5 um); mobile phase, Hex(0.2% IPA.M):EtOH=65:35; Flowing rate: 20 ml/min. This results in Compound 19A (the first eluting isomer, Retention Time 4.983 min) and Compound 19B (the second eluting isomer, Retention Time 6.238 min).

### Example 20

### 5-ethynyl-6-fluoro-4-(8-fluoro-2-((1-(morpholinomethyl)cyclopropyl)methoxy)-4-(1,4-oxazepan-4-yl)quinazolin-7-yl)naphthalen-2-ol trifluoroacetic acid (“Compound 20”)

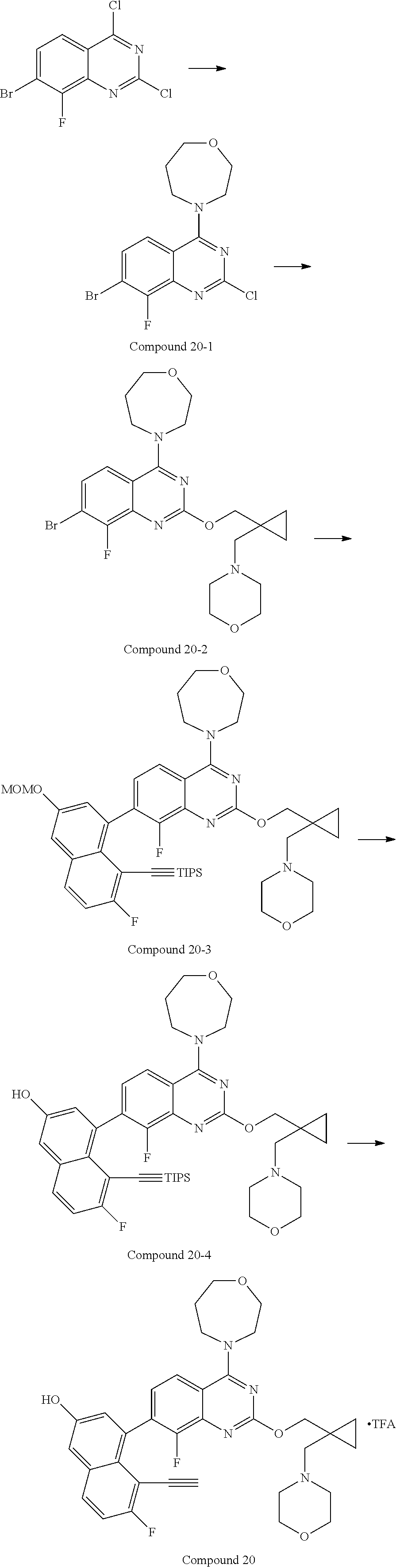

A solution of 7-bromo-2,4-dichloro-8-fluoroquinazoline (408 mg, 1.3787 mmol), 1,4-oxazepane (156 mg, 1.5423 mmol) and DIEA (543 mg, 4.2014 mmol) in DCM (10 mL) was stirred 5 h at room temperature. More 1,4-oxazepane (156 mg, 1.5423 mmol) were added and then the mixture was stirred at room temperature for 16 h. The solution was diluted with saturated NaHCO₃ aqueous solution (10 mL). The organic layer was washed with saturated NaCl aqueous solution, dried over anhydrous Na₂SO₄ and concentrated in vacuum to give Compound 20-1 (436 mg, 1.2091 mmol, 87.6938% yield). MS m/z: 360 [M+H]⁺.

A solution of Compound 20-1 (0.211 g, 585.1216 μmol), (1-(morpholinomethyl)cyclopropyl)methanol (121 mg, 706.6267 μmol), triethylene diamine (40 mg, 356.5952 μmol), cesium carbonate (241 mg, 739.6747 μmol) in THF (4 mL) and N,N-dimethylformamide (4 mL) was stirred at room temperature for 18 h under nitrogen atmosphere. The mixture was diluted with saturated NaHCO₃ aqueous solution (10 mL) and extracted with EA (10 mL). The organic layer was washed with 10 mL saturated NaCl aqueous solution, then dried over anhydrous Na₂SO₄ and concentrated in vacuum. The residue was purified by Pre-TLC to give Compound 20-2 (294 mg, 593.4790 μmol, 101.4283% yield). MS m/z: 495 [M+H]⁺.

A solution of Compound 20-2 (294 mg, 593.4790 μmol), INT 3 (276 mg, 538.5002 μmol), cataCXium A Pd G3 (45 mg, 61.7903 μmol), potassium phosphate (386 mg, 1.8185 mmol) in toluene (5 mL) and water (1 mL) was stirred at 100° C. for 16 hours under nitrogen atmosphere. The mixture was allowed to cool to room temperature, diluted with saturated NaHCO₃ aqueous solution (10 mL) and extracted with EA (10 mL). The organic layer was washed with 10 mL saturated NaCl aqueous solution, then dried over anhydrous Na₂SO₄ and concentrated in vacuum. The residue was purified by Pre-TLC to give Compound 20-3 (221 mg, 275.8893 mol, 46.4868% yield). MS m/z: 801 [M+H]⁺.

A solution of Compound 20-3 (0.221 g, 275.8893 μmol) and HCl (4 M in dioxane, 1 mL) in DCM (10 mL) was stirred at room temperature for 1 h. The solution was diluted with DCM (20 mL) and 10% NaHCO₃ solution (20 mL). The organic layer was washed with saturated NaCl aqueous solution, dried over anhydrous Na₂SO₄ and concentrated in vacuum to give Compound 20-4 (213 mg, 281.3763 μmol, 101.9888% yield). MS m/z: 757 [M+H]⁺.

A solution of Compound 20-4 (0.213 g, 281.3764 μmol) and CsF (323 mg, 2.1264 mmol) in DMF (8 mL) was stirred for 20 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 45% B in 37 min at a flow rate of 60 mL/min, 240 nm) and freeze-dried to give Compound 20 (105 mg, 168.5222 μmol, 43.4923% yield, TFA salt). MS m/z: 601 [M+H]⁺.

### Example 21

### 5-ethynyl-6-fluoro-4-(8-fluoro-4-(1,4-oxazepan-4-yl)-2-((tetrahydro-1H-pyrrolizin-7a(5H)-yl)methoxy)pyrido[4,3-d]pyrimidin-7-yl)naphthalen-2-ol trifluoroacetic acid (“Compound 21”)

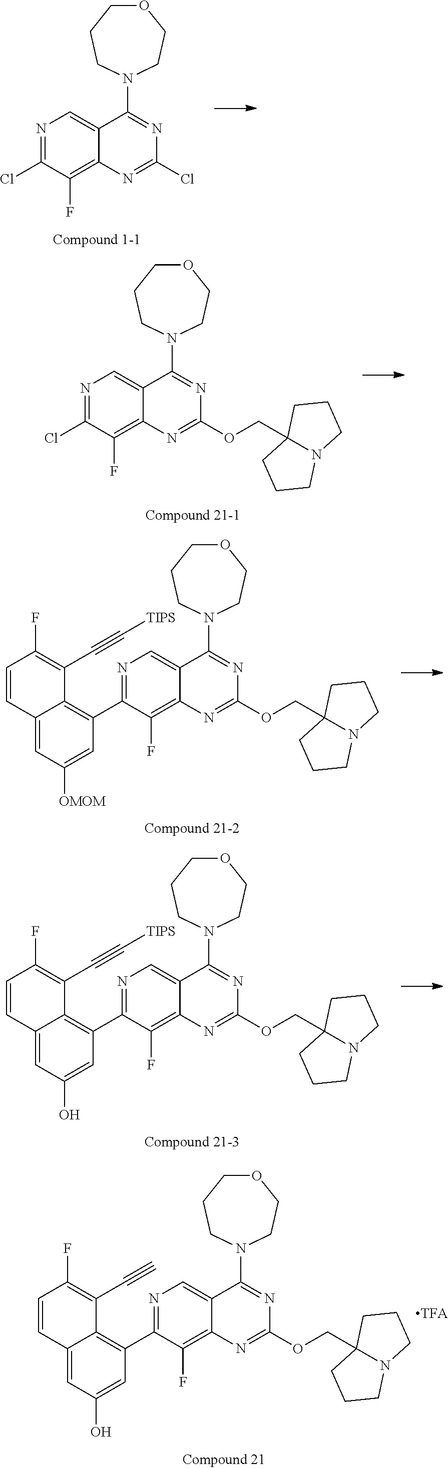

A solution of Compound 1-1 (0.232 g, 731.5245 μmol), (tetrahydro-1H-pyrrolizin-7a(5H)-yl)methanol (119 mg, 842.7153 μmol) and KF (134 mg, 2.3065 mmol) in DMSO (8 mL) was stirred at 85° C. for 20 h under nitrogen atmosphere. The mixture was allowed to cool to room temperature, diluted with saturated NaHCO₃ aqueous solution (10 mL) and extracted with EA (10 mL). The organic layer was washed with saturated NaCl aqueous solution, then dried over anhydrous Na₂SO₄ and concentrated in vacuum. The residue was purified by pre-TLC to give Compound 21-1 (121 mg, 286.8011 μmol, 39.2059% yield). MS m/z: 422 [M+H]⁺.

A solution of Compound 21-1 (58 mg, 137.4749 μmol), INT 3 (82 mg, 159.9892 μmol), cataCXium A Pd G3 (21 mg, 28.8355 μmol), potassium phosphate (146 mg, 448.1017 μmol) in toluene (2.5 mL) and water (0.5 mL) was stirred at 100° C. for 4 hours under nitrogen atmosphere. The mixture was allowed to cool to room temperature, diluted with saturated NaHCO₃ aqueous solution (10 mL) and extracted with EA (10 mL). The organic layer was washed with 10 mL saturated NaCl aqueous solution, then dried over anhydrous Na₂SO₄ and concentrated in vacuum. The residue was purified by Pre-TLC to give Compound 21-2 (78 mg, 101.0352 μmol, 73.4936% yield). MS m/z: 772 [M+H]⁺.

A solution of Compound 21-2 (0.078 g, 101.0352 μmol) and HCl (4 M in dioxane, 1 mL) in DCM (10 mL) was stirred at room temperature for 1 h. The solution was diluted with DCM (10 mL) and 10% NaHCO₃ solution (10 mL). The organic layer was washed with saturated NaCl aqueous solution, dried over anhydrous Na₂SO₄ and concentrated in vacuum to give Compound 21-3 (66 mg, 90.6649 μmol, 89.7359% yield). MS m/z: 728 [M+H]⁺.

A solution of Compound 21-3 (0.066 g, 90.6649 μmol) and CsF (107 mg, 704.3950 μmol) in DMF (8 mL) was stirred for 20 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 33 min at a flow rate of 70 mL/min, 230 nm) and freeze-dried to give Compound 21 (24 mg, 41.9862 mol, 46.3093% yield, TFA salt). MS m/z: 572 [M+H]⁺.

### Example 22

### 5-ethynyl-6-fluoro-4-(8-fluoro-2-(((2R,7aS)-2-fluorotetrahydro-1H-pyrrolizin-7a(5H)-yl)methoxy)-4-(5-methyl-1,4-oxazepan-4-yl)pyrido[4,3-d]pyrimidin-7-yl)naphthalen-2-ol trifluoroacetic acid (“Compound 22”)

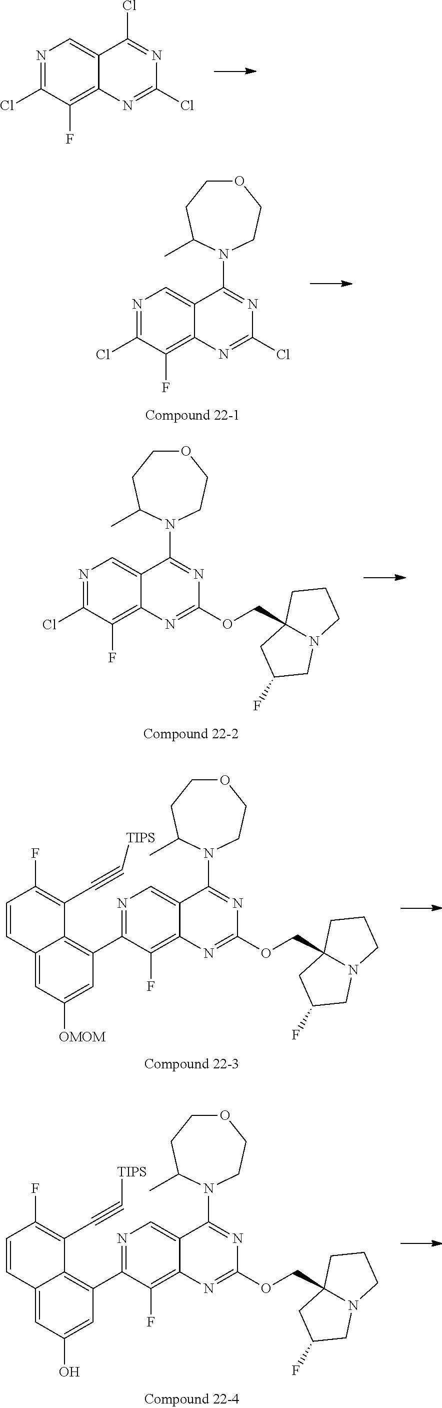

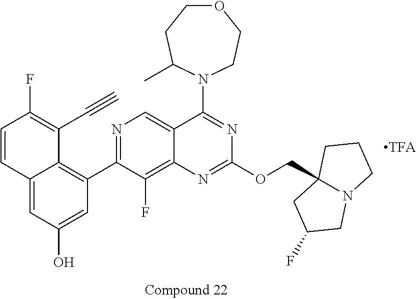 [SMILES: C#Cc1c(F)ccc2cc(O)cc(-c3ncc4c(N5CCOCCC5C)nc(OC[C@@]56CCCN5C[C@H](F)C6)nc4c3F)c12]

5-methyl-1,4-oxazepane hydrochloride (365 mg, 2.4071 mmol) was added in several batches into the solution of INT 1 (305 mg, 1.2081 mmol) and DIEA (553 mg, 4.2788 mmol) in DCM (10 mL), and then the mixture was stirred at 0° C. for 2 h. The solution was diluted with 10% NaHCO₃ solution. The organic layer was washed with saturated NaCl aqueous solution, dried over anhydrous Na₂SO₄ and concentrated in vacuum. The residue was slurried with EA (2 mL), Hex (20 mL) to give Compound 22-1 (354 mg, 1.0689 mmol, 88.4794% yield). MS m/z: 331 [M+H]⁺.

A solution of Compound 22-1 (0.354 g, 1.0689 mmol), INT 2 (195 mg, 1.2249 mmol) and potassium fluoride (193 mg, 3.3220 mmol) in DMSO (10 mL) was stirred at 100° C. for 18 h under nitrogen atmosphere. The mixture was diluted with saturated NaHCO₃ aqueous solution (15 mL) and extracted with EA (15 mL). The organic layer was washed with saturated NaCl aqueous solution, then dried over anhydrous Na₂SO₄ and concentrated in vacuum. The residue was purified by Pre-TLC to give Compound 22-2 (387 mg, 852.5878 μmol, 79.7609% yield). MS m/z: 454 [M+H]⁺.

A solution of Compound 22-2 (98 mg, 215.9008 μmol), INT 3 (150 mg, 292.6632 μmol), cataCXium A Pd G3, (30 mg, 41.1935 μmol), potassium phosphate (224 mg, 687.4985 μmol) in toluene (5 mL) and water (1 mL) was stirred at 100° C. for 16 hours under nitrogen atmosphere. The mixture was allowed to cool to room temperature, diluted with saturated NaHCO₃ aqueous solution (15 mL) and extracted with EA (15 mL). The organic layer was washed with 10 mL saturated NaCl aqueous solution, then dried over anhydrous Na₂SO₄ and concentrated in vacuum. The residue was purified by Pre-TLC to give Compound 22-3 (122 mg, 151.7366 μmol, 70.2807% yield). MS m/z: 804 [M+H]⁺.

A solution of Compound 22-3 (0.122 g, 151.7366 μmol) and HCl (4 M in dioxane, 1 mL) in DCM (10 mL) was stirred at room temperature for 2 h. The solution was diluted with 10% NaHCO₃ solution (20 mL). The organic layer was washed with saturated NaCl aqueous solution, dried over anhydrous Na₂SO₄ and concentrated in vacuum to give Compound 22-4 (128 mg, 168.4271 μmol, 110.9997% yield). MS m/z: 760 [M+H]⁺.

A solution of Compound 22-4 (0.128 g, 168.4271 μmol) and CsF (142 mg, 934.8046 μmol) in DMF (10 mL) was stirred for 20 hours at 40° C. under nitrogen atmosphere. The solution was diluted with water (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 42 min at a flow rate of 60 mL/min, 235 nm) and freeze-dried to give Compound 22 (20 mg, 33.1327 μmol, 19.6718% yield, TFA salt). MS m/z: 604 [M+H]⁺.

### Example 23

### 4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol trifluoroacetic acid (“Compound 23”)

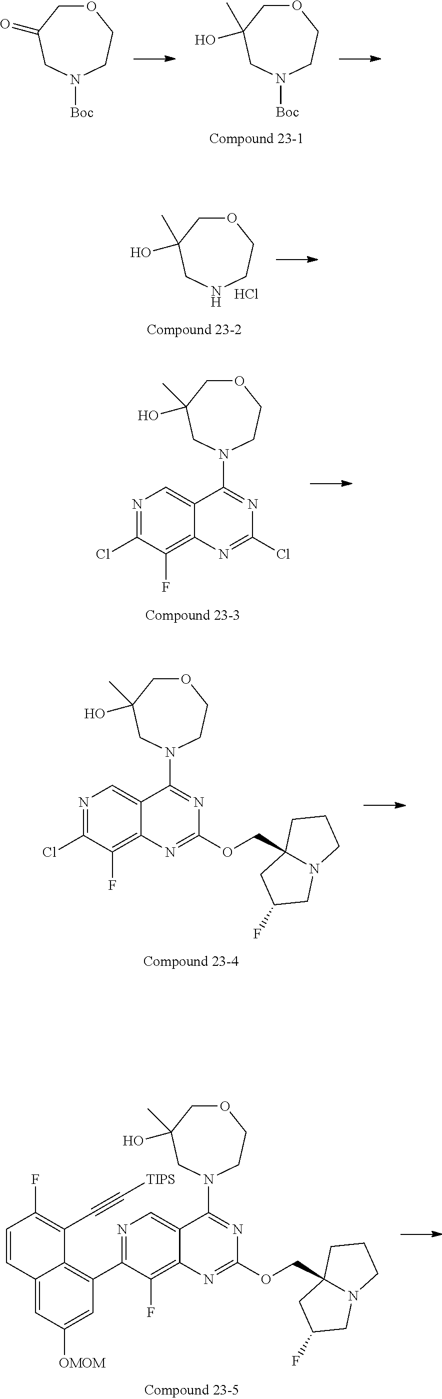

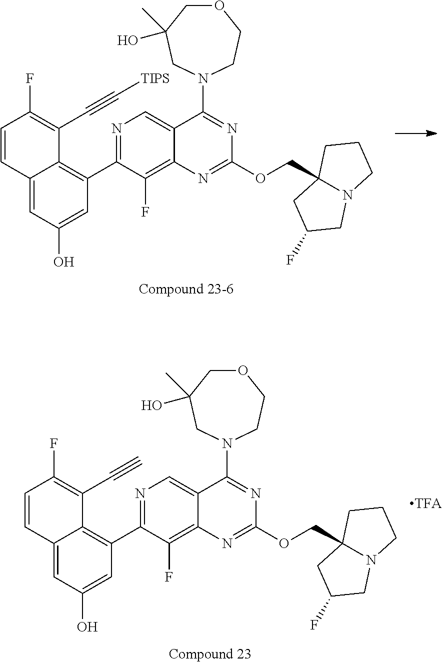

Methyl magnesium bromide (20 mL, 1.0 M in THF) was dropped into a solution of tert-butyl 6-oxo-1,4-oxazepane-4-carboxylate (2.88 g, 13.3801 mmol) in THF (50 mL), and then the mixture was stirred at 0° C. for 4 h. The solution was quenched with saturated NH₄Cl aqueous solution (50 mL). The organic layer was washed with saturated NaCl aqueous solution (50 mL), dried over anhydrous Na₂SO₄ and concentrated in vacuum to give Compound 23-1 (2726 mg, 11.7862 mmol, 88.0876% yield). MS m/z: 216 [M+H]⁺.

A solution of Compound 23-1 (0.074 g, 99.4717 μmol) and hydrogen chloride (10 mL) in DCM (10 mL) was stirred for 20 hours at room temperature. The system was filtered and the filter cake was dried to give Compound 23-2 (476 mg, 3.6288 mmol, 110.5798% yield). MS m/z: 116 [M+H]⁺.

DIEA (317 mg, 2.4528 mmol) was dropped into the solution of INT 1 (201 mg, 796.1657 μmol) and Compound 23-2 (132 mg, 1.0063 mmol) in DCM (10 mL), and the mixture was stirred at 0° C. for 1 h. The solution was diluted with 10% citric acid (10 mL). The organic layer was washed with saturated NaCl aqueous solution, dried over anhydrous Na₂SO₄ and concentrated in vacuum to give Compound 23-3 (276 mg, 794.9954 μmol, 99.8530% yield). MS m/z: 347 [M+H]⁺.

A solution of Compound 23-3 (0.276 g, 794.9950 μmol), INT 2 (167 mg, 1.0490 mmol) and potassium fluoride (154 mg, 2.6508 mmol) in DMSO (10 mL) was stirred at 100° C. for 18 h under nitrogen atmosphere. The mixture was diluted with saturated NaHCO₃ aqueous solution (15 mL) and extracted with EA (15 mL). The organic layer was washed with saturated NaCl aqueous solution, then dried over anhydrous Na₂SO₄ and concentrated in vacuum. The residue was purified by Pre-TLC to give Compound 23-4 (150 mg, 319.2090 μmol, 40.1523% yield). MS m/z: 470 [M+H]⁺.

A solution of Compound 23-4 (0.15 g, 319.2090 μmol), INT 3 (241 mg, 470.2122 μmol), cataCXium A Pd G3 (67 mg, 91.9988 μmol), potassium phosphate (329 mg, 1.0098 mmol) in toluene (10 mL) and water (2 mL) was stirred at 100° C. for 16 hours under nitrogen atmosphere. The mixture was allowed to cool to room temperature, diluted with water (15 mL) and extracted with EA (15 mL). The organic layer was washed with 10 mL saturated NaCl aqueous solution, then dried over anhydrous Na₂SO₄ and concentrated in vacuum. The residue was purified by Pre-TLC to give Compound 23-5 (116 mg, 141.4592 μmol, 44.3155% yield). MS m/z: 820 [M+H]⁺.

A solution of Compound 23-5 (0.116 g, 141.4593 μmol) and HCl (4 M in dioxane, 1 mL) in DCM (10 mL) was stirred at room temperature for 1 h. The solution was diluted with 10% NaHCO₃ solution (20 mL). The organic layer was washed with saturated NaCl aqueous solution, dried over anhydrous Na₂SO₄ and concentrated in vacuum to give Compound 23-6 (120 mg, 154.6448 μmol, 109.3210% yield). MS m/z: 776 [M+H]⁺.

A solution of Compound 23-6 (0.12 g, 154.6448 μmol) and CsF (150 mg, 987.4697 μmol) in DMF (10 mL) was stirred for 20 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 10% B to 27% B in 26 min at a flow rate of 60 mL/min, 230 nm) and freeze-dried to give Compound 23 (18 mg, 29.0495 μmol, 18.7847% yield, TFA salt). MS m/z: 620 [M+H]⁺.

### Example 24

### 5-ethynyl-6-fluoro-4-(8-fluoro-2-(((2R,7aS)-2-fluorotetrahydro-1H-pyrrolizin-7a(5H)-yl)methoxy)-4-(6-methylene-1,4-oxazepan-4-yl)pyrido[4,3-d]pyrimidin-7-yl)naphthalen-2-ol (“Compound 24”)

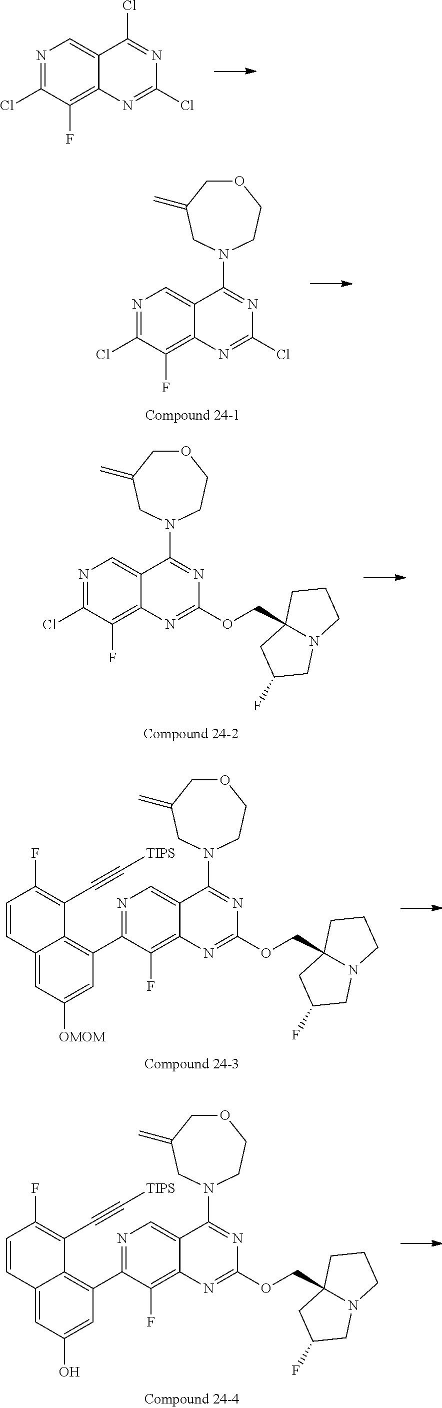

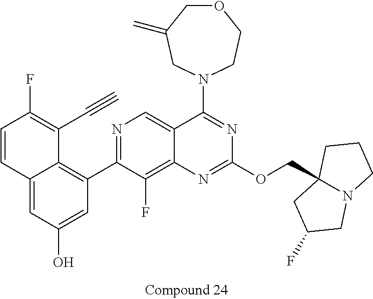 [SMILES: C#Cc1c(F)ccc2cc(O)cc(-c3ncc4c(N5CCOCC(=C)C5)nc(OC[C@@]56CCCN5C[C@H](F)C6)nc4c3F)c12]

6-Methylene-[1,4]oxazepane hydrochloride (86 mg, 574.7968 μmol) was added in several batches into the solution of INT 1 (146 mg, 578.3087 μmol) and DIEA (271 mg, 2.0968 mmol) in DCM (10 mL), and the mixture was stirred at 0° C. for 1 h. The solution was diluted with 10% citric acid (10 mL). The organic layer was washed with saturated NaCl aqueous solution, dried over anhydrousvNa₂SO₄ and concentrated in vacuum to give Compound 24-1 (211 mg, 641.0323 μmol, 110.8460% yield). MS m/z: 329 [M+H]⁺.

A solution of Compound 24-1 (0.211 g, 641.0323 μmol), INT 2 (144 mg, 904.5186 μmol) and potassium fluoride (110 mg, 1.8934 mmol) in DMSO (10 mL) was stirred at 100° C. for 18 h under nitrogen atmosphere. The mixture was diluted with saturated NaHCO₃ aqueous solution (15 mL) and extracted with EA (15 mL). The organic layer was washed with saturated NaCl aqueous solution, then dried over anhydrous Na₂SO₄ and concentrated in vacuum. The residue was purified by Pre-TLC to give Compound 24-2 (171 mg, 378.4053 μmol, 59.0306% yield). MS m/z: 452 [M+H]⁺.

A solution of Compound 24-2 (0.171 g, 378.4053 μmol), INT 3 (251 mg, 489.7230 μmol), cataCXium A Pd G3 (50 mg, 68.6559 μmol), potassium phosphate (371 mg, 1.1387 mmol) in toluene (10 mL) and water (2 mL) was stirred at 100° C. for 16 hours under nitrogen atmosphere. The mixture was allowed to cool to room temperature, diluted with water (15 mL) and extracted with EA (15 mL). The organic layer was washed with 10 mL saturated NaCl aqueous solution, then dried over anhydrous Na₂SO₄ and concentrated in vacuum. The residue was purified by Pre-TLC to give Compound 24-3 (228 mg, 284.2860 μmol, 75.1274% yield). MS m/z: 802 [M+H]⁺.

A solution of Compound 24-3 (0.228 g, 284.2861 μmol) and HCl (4 M in dioxane, 2 mL) in DCM (10 mL) was stirred at room temperature for 1 h. The solution was diluted with 10% NaHCO₃ solution (15 mL). The organic layer was washed with saturated NaCl aqueous solution, dried over anhydrous Na₂SO₄ and concentrated in vacuum to give Compound 24-4 (276 mg, 364.1368 μmol, 128.0882% yield). MS m/z: 758 [M+H]⁺.

A solution of Compound 24-4 (0.276 g, 364.1368 μmol) and CsF (276.5681 mg, 1.8207 mmol) in DMF (10 mL) was stirred for 20 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 10% B to 30% B in 29 min at a flow rate of 60 mL/min, 235 nm). The eluent was adjusted to pH=8 and the acetonitrile in the eluent was concentrated. The resulting aqueous phase was extracted with DCM and the organic phase was dried, concentrated and freeze-dried to give Compound 24 (56 mg, 93.0825 μmol, 25.5625% yield). MS m/z: 602 [M+H]⁺.

### Example 25

### (E)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one O-methyl oxime (“Compound 25”)

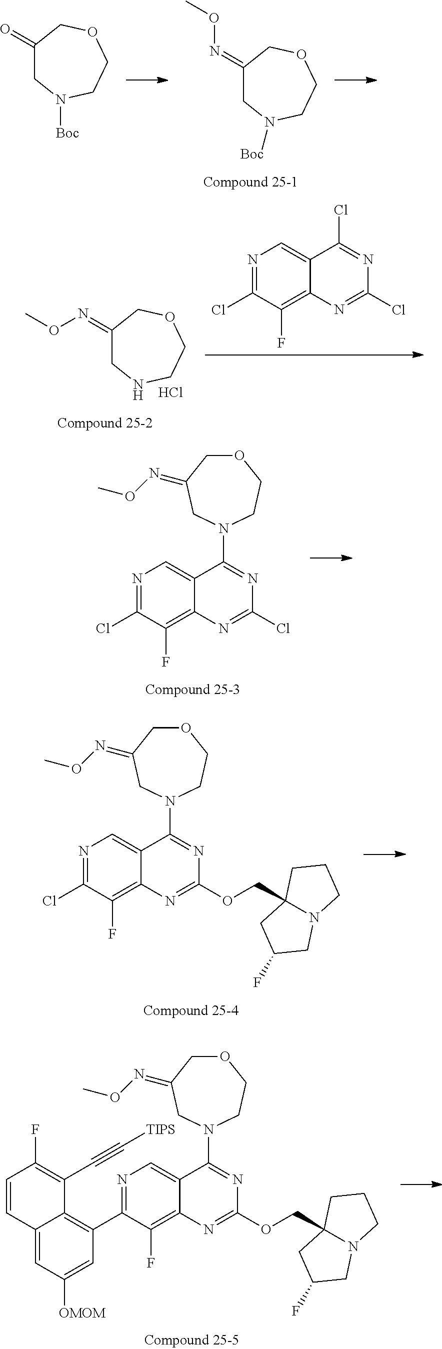

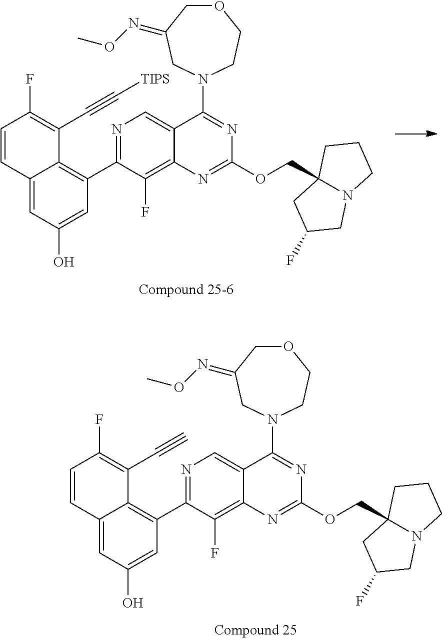

A solution of tert-butyl 6-oxo-1,4-oxazepane-4-carboxylate (600 mg, 2.7875 mmol), methoxamine hydrochloride (349.2080 mg, 4.1813 mmol) and pyridine (330.7372 mg, 4.1813 mmol) in methanol (10 mL) was stirred at room temperature for 3 h. The solution was concentrated in vacuum, then diluted with water (20 mL) and extracted with EA (20 mL). The organic layer was washed with saturated NaCl aqueous solution, dried over anhydrous Na₂SO₄ and concentrated in vacuum to give Compound 25-1 (664 mg, 2.7181 mmol, 97.5105% yield). MS m/z: 245 [M+H]⁺.

A solution of Compound 25-1 (0.664 g, 2.7181 mmol) and HCl (4M in 1,4-dioxane, 1 mL) in DCM (10 mL) was stirred for 20 hours at room temperature, then the mixture was filtered and the filter cake was dried to give Compound 25-2 (311 mg, 2.1572 mmol, 79.3623% yield). MS m/z: 145 [M+H]⁺.

Compound 25-2 (147 mg, 813.8084 μmol) was added in several batches into the solution of INT 1 (212 mg, 839.7360 μmol) and DIEA (322 mg, 2.4914 mmol) in DCM (10 mL), and the mixture was stirred at 0° C. for 3 h. The solution was diluted with 10% citric acid (20 mL). The organic layer was washed with saturated NaCl aqueous solution (20 mL), dried over anhydrous Na₂SO₄ and concentrated in vacuum to give Compound 25-3 (305 mg, 846.8209 μmol, 100.8437% yield). MS m/z: 360 [M+H]⁺.

A solution of Compound 25-3 (0.305 g, 846.8209 μmol), INT 2 (180 mg, 1.1306 mmol) and potassium fluoride (163 mg, 2.8057 mmol) in DMSO (10 mL) was stirred at 100° C. for 18 h under nitrogen atmosphere. The mixture was diluted with saturated NaHCO₃ aqueous solution (20 mL) and extracted with EA (20 mL). The organic layer was washed with saturated NaCl aqueous solution, then dried over anhydrous Na₂SO₄ and concentrated in vacuum. The residue was purified by Pre-TLC to give Compound 25-4 (168 mg, 347.8906 μmol, 41.0820% yield). MS m/z: 483 [M+H]⁺.

A solution of Compound 25-4 (0.168 g, 347.8909 μmol), INT 3 (267.4592 mg, 521.8364 μmol), cataCXium A Pd G3 (50.6717 mg, 69.5782 μmol), potassium phosphate (340.0483 mg, 1.0437 mmol) in toluene (10 mL) and water (2 mL) was stirred at 100° C. for 16 hours under nitrogen atmosphere. The mixture was allowed to cool to room temperature, diluted with water (15 mL) and extracted with EA (15 mL). The organic layer was washed with saturated NaCl aqueous solution, then dried over anhydrous Na₂SO₄ and concentrated in vacuum. The residue was purified by Pre-TLC to give Compound 25-5 (118 mg, 141.6527 mol, 40.7176% yield). MS m/z: 833 [M+H]⁺.

A solution of Compound 25-5 (0.118 g, 141.6528 μmol) and HCl (4 M in dioxane, 1 mL) in DCM (10 mL) was stirred at room temperature for 1 h. The solution was diluted with 10% NaHCO₃ solution (10 mL). The organic layer was washed with saturated NaCl aqueous solution, dried over anhydrous Na₂SO₄ and concentrated in vacuum to give Compound 25-6 (130 mg, 164.7716 μmol, 116.3208% yield). MS m/z: 789 [M+H]⁺.

A solution of Compound 25-6 (0.13 g, 164.7716 μmol) and CsF (123 mg, 809.7251 μmol) in DMF (10 mL) was stirred for 20 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 46% B in 47 min at a flow rate of 60 mL/min, 230 nm). The eluent was adjusted to pH=8 and the acetonitrile in the eluent was concentrated. The resulting aqueous phase was extracted with DCM and the organic phase was dried, concentrated and freeze-dried to give Compound 25 (30 mg, 47.4210 μmol, 28.7798% yield). MS m/z: 633 [M+H]⁺.

### Example 26

### 4-(4-(6-(dimethylamino)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 26”)

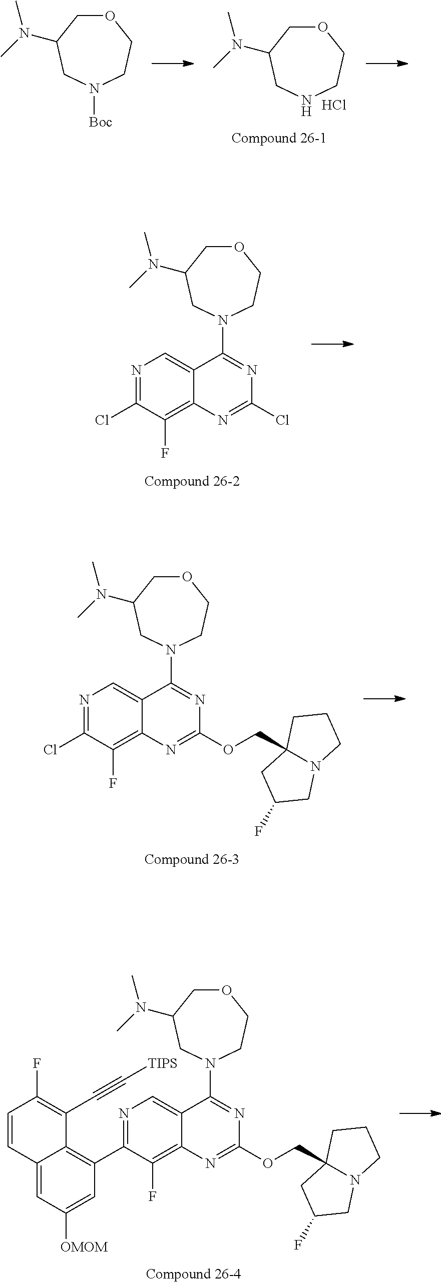

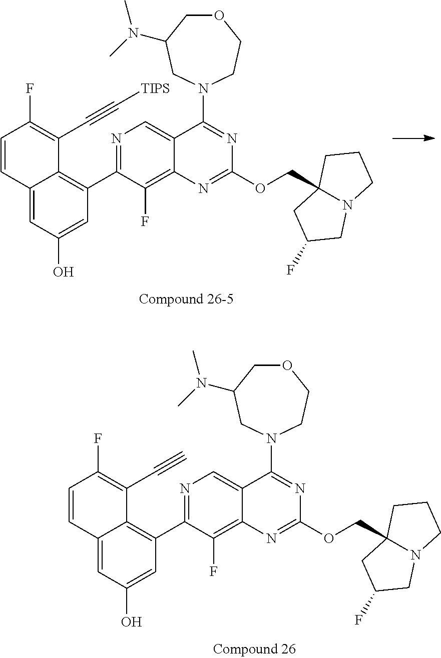

A solution of tert-butyl 6-(dimethylamino)-1,4-oxazepane-4-carboxylate (158 mg, 646.6664 mmol), HCl (4 M in dioxane, 0.5 mL) in DCM (3 mL) was stirred for 20 hours at room temperature and was concentrated in vacuum to give crude Compound 26-1 (93 mg, 644.8747 mmol, 99.7229% yield). MS m/z: 145 [M+H]⁺.

Compound 26-1 (93 mg, 644.8747 mmol) was dropped into the solution of 2,4,7-trichloro-8-fluoropyrido[4,3-d]pyrimidine (182 mg, 720.9063 μmol), DIEA (286 mg, 2.2129 mmol) in DCM (10 mL), and was stirred at 0° C. for 1 h. The solution was diluted with 10% citric acid (10 mL), the organic layer was washed with saturated NaCl aqueous solution, dried over anhydrous Na₂SO₄ and concentrated in vacuum. The residue was purified by pre-TLC to give Compound 26-2 (68 mg, 188.7769 μmol, 26.1861% yield). MS m/z: 360 [M+H]⁺.

A solution of Compound 26-2 (68 mg, 188.7769 μmol), INT 2 (152 mg, 954.7679 μmol), potassium fluoride (46 mg, 791.7834 mmol) in DMSO (2 mL) was stirred at 100° C. for 18 h under nitrogen atmosphere. The mixture was diluted with saturated NaCl aqueous solution (100 mL) and extracted with EA (50 mL). The organic layer was dried over anhydrous Na₂SO₄ and concentrated in vacuum. The residue was purified by Pre-TLC to give Compound 26-3 (69 mg, 142.8710 μmol, 75.6824% yield). MS m/z: 483 [M+H]⁺.

A solution of Compound 26-3 (69 mg, 142.8710 μmol), INT 3 (96 mg, 187.3044 μmol), cataCXium A Pd G3 (20 mg, 27.4623 μmol), potassium phosphate (152 mg, 466.5168 μmol) in toluene (3 mL) and water (0.6 mL) was stirred at 100° C. for 16 hours under nitrogen atmosphere. The mixture was allowed to cool to room temperature and diluted with water (10 mL) and extracted with DCM (20 mL). The organic layer was dried over anhydrous Na₂SO₄ and concentrated in vacuum. The residue was purified by Pre-TLC to give Compound 26-4 (77 mg, 92.4296 μmol, 64.6945% yield). MS m/z: 833 [M+H]⁺.

A solution of Compound 26-4 (0.077 g, 92.4297 μmol), HCl (4 M in dioxane, 1 mL) in DCM (10 mL) was stirred at RT for 1 h. The solution was diluted with 10% NaHCO₃ solution (20 mL), the organic layer was washed with saturated NaCl aqueous solution, dried over anhydrous Na₂SO₄ and concentrated in vacuum to give Compound 26-5 (58 mg, 73.5095 μmol, 79.5302% yield). MS m/z: 789 [M+H]⁺.

A solution of Compound 26-5 (0.058 g, 73.5095 μmol), CsF (56 mg, 368.6553 μmol) in DMF (10 mL) was stirred for 20 hours at 40° C. under nitrogen atmosphere. The solution was diluted with water (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 30% B in 30 min at a flow rate of 60 mL/min, 235 nm). The eluent was adjusted to pH=8 and the acetonitrile in the eluent was concentrated. The resulting aqueous phase was extracted with DCM and the organic phase was dried, concentrated and freeze-dried to give Compound 26 (23 mg, 36.3536 μmol, 49.4544% yield). MS m/z: 633 [M+H]⁺.

### Example 27

### 4-(4-(3,6-dimethyl-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 27”)

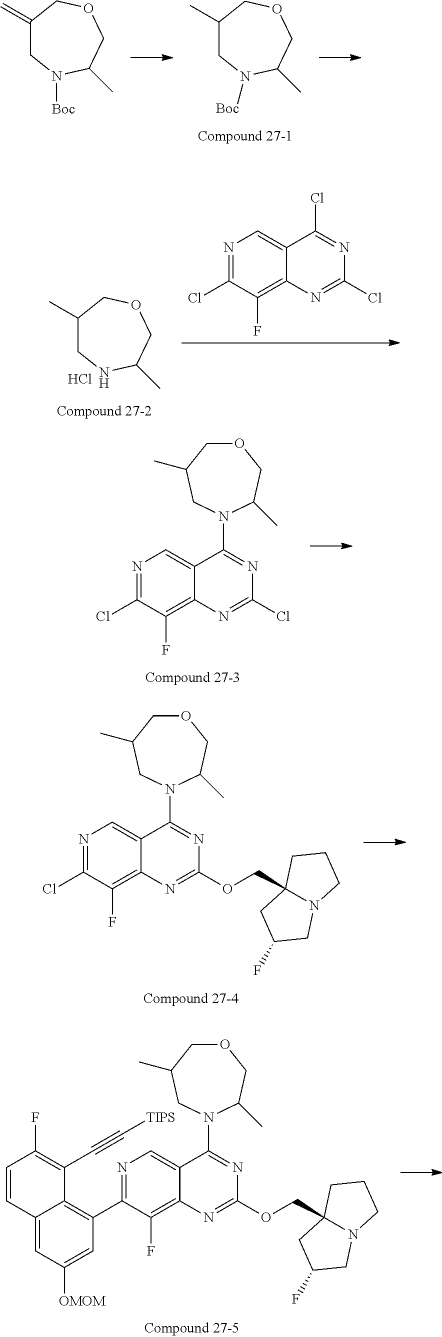

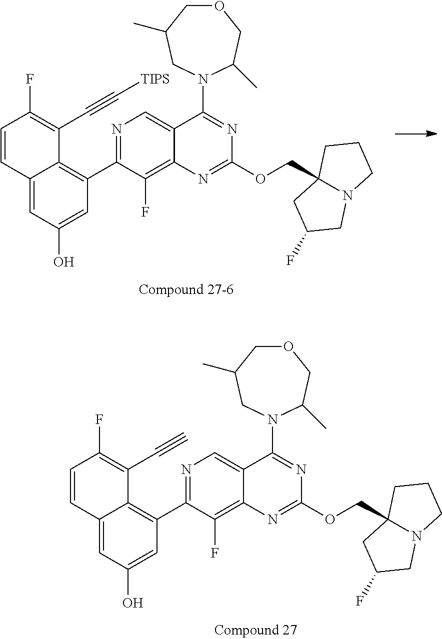

A solution of tert-butyl 3-methyl-6-methylene-1,4-oxazepane-4-carboxylate (0.207 g, 910.6947 μmol) and Pd/C (43 mg) in methanol (8 mL) was stirred for 4 hours at room temperature under H₂ atmosphere and the mixture was filtered, then the filtrate was concentrated in vacuum to give Compound 27-1 (205 mg, 893.9668 μmol). MS m/z: 230 [M+H]⁺.

A solution of Compound 27-1 (0.205 g, 893.9668 μmol) and HCl (4 M in dioxane, 0.5 mL) in DCM (10 mL) was stirred for 20 hours at room temperature and the mixture was concentrated in vacuum to give crude Compound 27-2 (169 mg, 1.0202 mmol, 114.1159% yield). MS m/z: 130 [M+H]⁺.

A solution of INT 1 (255 mg, 1.0101 mmol), Compound 27-2 (0.169 g, 1.0202 mmol) and DIEA (405 mg, 3.1336 mmol) in DCM (10 mL) was stirred at 0° C. for 5 h. The solution was diluted with 5% citric acid (10 mL). The organic layer was washed with saturated NaCl aqueous solution, dried over anhydrous Na₂SO₄ and concentrated in vacuum. The residue was purified by pre-TLC to give Compound 27-3 (276 mg, 799.5390 μmol, 78.3742% yield). MS m/z: 345 [M+H]⁺.

A solution of Compound 27-3 (0.276 g, 799.5390 μmol), INT 2 (194 mg, 1.2186 mmol) and potassium fluoride (139 mg, 2.3926 mmol) in DMSO (10 mL) was stirred at 100° C. for 18 h under nitrogen atmosphere. The mixture was diluted with water (10 mL) and extracted with EA (10 mL). The organic layer was washed with saturated NaCl aqueous solution, then dried over anhydrous Na₂SO₄ and concentrated in vacuum. The residue was purified by Pre-TLC to give Compound 27-4 (232 mg, 495.7914 μmol, 62.0097% yield). MS m/z: 468 [M+H]⁺.

A solution of Compound 27-4 (232 mg, 495.7914 μmol), INT 3 (364 mg, 710.1960 μmol), cataCXium A Pd G3, (70 mg, 96.1182 μmol), potassium phosphate (494 mg, 1.5162 mmol) in toluene (7.5 mL) and water (1.5 mL) was stirred at 100° C. for 16 hours under nitrogen atmosphere. The mixture was allowed to cool to room temperature, diluted with water (10 mL) and extracted with EA (10 mL). The organic layer was dried over anhydrous Na₂SO₄ and concentrated in vacuum. The residue was purified by Pre-TLC to give Compound 27-5 (287 mg, 350.8337 μmol, 70.7623% yield). MS m/z: 818 [M+H]⁺.

A solution of Compound 27-5 (0.287 g, 350.8334 μmol) and HCl (4 M in dioxane, 1 mL) in DCM (10 mL) was stirred at room temperature for 1 h. The solution was diluted with 10% NaHCO₃ solution (20 mL) and extracted with DCM (1 mL). The organic layer was washed with saturated NaCl aqueous solution (20 mL), dried over anhydrous Na₂SO₄ and concentrated in vacuum to give Compound 27-6 (325 mg, 419.8971 μmol, 119.6856% yield). MS m/z: 774 [M+H]⁺.

A solution of Compound 27-6 (325 mg, 419.8971 μmol) and CsF (299 mg, 1.9684 mmol) in DMF (10 mL) was stirred for 20 hours at 40° C. under nitrogen atmosphere. The solution was diluted with water (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 48% B in 37 min at a flow rate of 60 mL/min, 230 nm). The eluent was adjusted to pH=8 and the acetonitrile in the eluent was concentrated. The resulting aqueous phase was extracted with DCM and the organic phase was dried, concentrated and freeze-dried to give Compound 27 (40 mg, 64.7606 μmol, 15.4230% yield). MS m/z: 618 [M+H]⁺.

### Example 28

### 4-(4-(2-oxa-6-azabicyclo[5.1.0]octan-6-yl)-8-fluoro-2-((1-(morpholinomethyl)cyclopropyl)methoxy)pyrido[4,3-d]pyrimidin-7-yl)-5-ethynyl-6-fluoronaphthalen-2-ol trifluoroacetic acid (“Compound 28”)

### 4-(4-((1S,7R)-2-oxa-6-azabicyclo[5.1.0]octan-6-yl)-8-fluoro-2-((1-(morpholinomethyl)cyclopropyl)m ethoxy)pyrido[4,3-d]pyrimidin-7-yl)-5-ethynyl-6-fluoronaphthalen-2-ol (“Compound 28A or Compound 28B”)

### 4-(4-((1R,7S)-2-oxa-6-azabicyclo[5.1.0]octan-6-yl)-8-fluoro-2-((1-(morpholinomethyl)cyclopropyl)methoxy)pyrido[4,3-d]pyrimidin-7-yl)-5-ethynyl-6-fluoronaphthalen-2-ol (“Compound 28B” or “Compound 28A”)

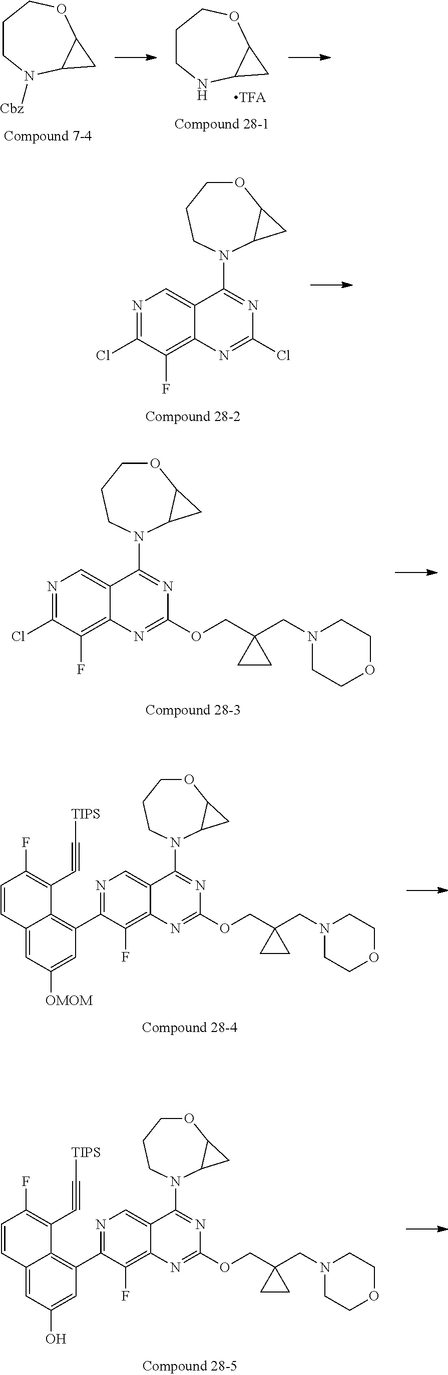

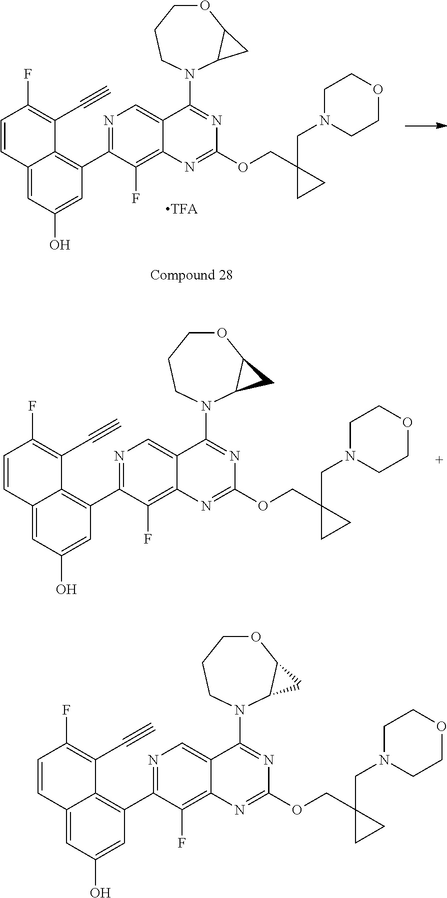 [SMILES: C#Cc1c(F)ccc2cc(O)cc(-c3ncc4c(N5CCCOC6CC65)nc(OCC5(CN6CCOCC6)CC5)nc4c3F)c12 ; C#Cc1c(F)ccc2cc(O)cc(-c3ncc4c(N5CCCO[C@H]6C[C@H]65)nc(OCC5(CN6CCOCC6)CC5)nc4c3F)c12 ; C#Cc1c(F)ccc2cc(O)cc(-c3ncc4c(N5CCCO[C@@H]6C[C@@H]65)nc(OCC5(CN6CCOCC6)CC5)nc4c3F)c12]

A solution of Compound 7-4 (1004 mg, 4.0600 mmol), di-tert-butyl dicarbonate (1043 mg, 4.7790 mmol) and Pd/C (370 mg) in EA (10 mL) was stirred for 20 hours at room temperature under H₂ atmosphere. The system was filtered and the filtrate was concentrated in vacuum. The residue was added into the solution of TFA (2 mL) in DCM (10 mL), stirred for 20 hours at room temperature and concentrated in vacuum to give crude Compound 28-1 (1667 mg, 7.3378 mmol, 180.7325% yield). MS m/z: 114 [M+H]⁺.

Compound 28-1 (1.66 g, 7.3070 mmol) was dropped into a solution of INT 1 (1179 mg, 4.6700 mmol) and DIEA (1613 mg, 12.4804 mmol) in DCM (20 mL), and the mixture was stirred at 0° C. for 1 h. The solution was diluted with 10% citric acid (20 mL) aqueous solution. The organic layer was washed with saturated NaCl aqueous solution, dried over anhydrous Na₂SO₄ and concentrated in vacuum. The residue was purified by slurrying in EA/Hex=1/6 to give Compound 28-2 (1571 mg, 4.7728 mmol, 102.2005% yield). MS m/z: 329 [M+H]⁺.

A solution of Compound 28-2 (203 mg, 616.7277 μmol), (1-(morpholinomethyl)cyclopropyl)methanol (157 mg, 916.8627 μmol) and potassium fluoride (128 mg, 2.2032 mmol) in DMSO (10 mL) was stirred at 100° C. for 18 h under nitrogen atmosphere. The mixture was diluted with water (10 mL) and extracted with EA (10 mL). The organic layer was washed with saturated NaCl aqueous solution, dried over anhydrous Na₂SO₄ and concentrated in vacuum. The residue was purified by Pre-TLC to give Compound 28-3 (121 mg, 260.8142 μmol, 42.2900% yield). MS m/z: 464 [M+H]⁺.

A solution of Compound 28-3 (0.121 g, 260.8142 μmol), INT 3 (208 mg, 405.8263 μmol), cataCXium A Pd G3, (22 mg, 30.2086 μmol), potassium phosphate (258 mg, 791.8509 μmol) in toluene (7.5 mL) and water (1.5 mL) was stirred at 100° C. for 16 hours under nitrogen atmosphere. The mixture was allowed to cool to room temperature, diluted with water (15 mL) and extracted with EA (15 mL). The organic layer was dried over anhydrous Na₂SO₄ and concentrated in vacuum. The residue was purified by Pre-TLC to give Compound 28-4 (119 mg, 146.1836 μmol, 56.0490% yield). MS m/z: 814 [M+H]⁺.

A solution of Compound 28-4 (0.119 g, 146.1836 μmol) and HCl (4 M in dioxane, 1.5 mL) in DCM (10 mL) was stirred at room temperature for 1 h. The solution was diluted with 10% NaHCO₃ aqueous solution (20 mL). The organic layer was washed with saturated NaCl aqueous solution, dried over anhydrous Na₂SO₄ and concentrated in vacuum to give Compound 28-5 (133 mg, 172.7290 μmol, 118.1590% yield). MS m/z: 770 [M+H]⁺.

A solution of Compound 28-5 (0.133 g, 172.7290 μmol) and CsF (103 mg, 678.0625 μmol) in DMF (7 mL) was stirred for 20 hours at 35° C. under nitrogen atmosphere. The solution was diluted with water (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 35% B to 74% B in 32 min at a flow rate of 70 mL/min, 235 nm). The eluent was concentrated in vacuum and freeze-dried to give the title Compound 28 (34 mg, TFA salt). MS m/z: 614 [M+H]⁺.

The Compound Compound 28 was separated by Prep-HPLC-Gilson with the following conditions: Column, CHIRAL ART Cellulose-SB column (2 cm×25 cm, 5 um), mobile phase, Hex (0.1% diethylamine):EtOH=65:35; Flowing rate: 20 ml/min. This results in Compound 28A (the first eluting isomer, Retention Time 7.435 min) and Compound 28B (the second eluting isomer, Retention Time 9.299 min).

### Example 29

5-ethynyl-6,7-difluoro-4-(8-fluoro-2-(((2R,7aS)-2-fluorotetrahydro-1H-pyrrolizin-7a(5H-yl ethoxy)-4-(1,4-oxazepan-4-yl)pyrido[4,3-d]pyrimidin-7-yl)naphthalen-2-ol (“Compound 29”)

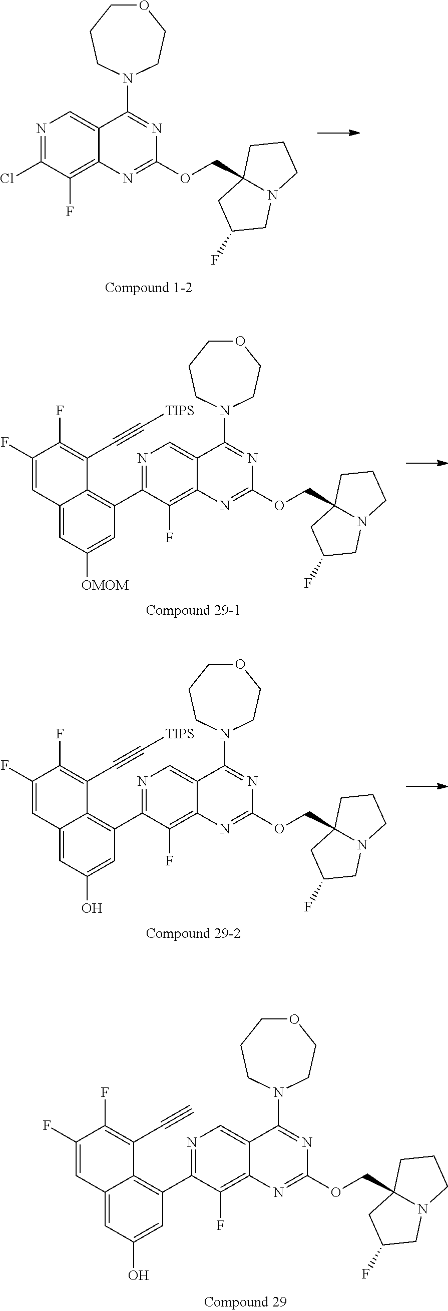

A solution of Compound 1-2 (138 mg, 313.7179 μmol), ((2,3-difluoro-6-(methoxymethoxy)-8-(4,4,5,5-tetramethyl-1,3,2-dioxaborolan-2-yl)naphthalen-1-yl)ethynyl)tri isopropylsilane (221 mg, 416.5684 μmol), cataCXium A Pd G3 (41 mg, 56.2978 μmol), potassium phosphate (314 mg, 963.7256 μmol) in toluene (10 mL) and water (2 mL) was stirred at 100° C. for 16 hours under nitrogen atmosphere. The mixture was allowed to cool to room temperature, diluted with saturated NaHCO₃ aqueous solution (15 mL) and extracted with EA (15 mL). The organic layer was washed with 10 mL NaCl aqueous solution, then dried over anhydrous Na₂SO₄ and concentrated in vacuum. The residue was purified by Pre-TLC to give Compound 29-1 (127 mg, 157.1804 μmol, 50.1025% yield). MS m/z: 808 [M+H]⁺.

A solution of Compound 29-1 (0.127 g, 157.1804 μmol) and HCl (4 M in dioxane 1 mL) in DCM (10 mL) was stirred at room temperature for 4 h. The solution was diluted with DCM (10 mL) and saturated NaHCO₃ aqueous solution solution (20 mL). The organic layer was washed with saturated NaCl aqueous solution, dried over anhydrous Na₂SO₄ and concentrated in vacuum to give Compound 29-2 (126 mg, 164.9352 μmol, 104.9337% yield). MS m/z: 764 [M+H]⁺.

A solution of Compound 29-2 (0.126 g, 164.9352 μmol) and CsF (135 mg, 888.7227 μmol) in DMF (5 mL) was stirred for 16 hours at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 42% B in 40 min at a flow rate of 60 mL/min, 230 nm).

The eluent was adjusted to pH=8 with NaHCO₃ aqueous solution and the acetonitrile in the eluent was concentrated. The resulting aqueous phase was extracted with DCM and the organic phase was dried, concentrated and freeze-dried to give Compound 29 (30 mg, 49.3748 μmol, 29.9359% yield). MS m/z: 608 [M+H]⁺.

### Example 30

### 4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one trifluoroacetic acid (“Compound 30”)

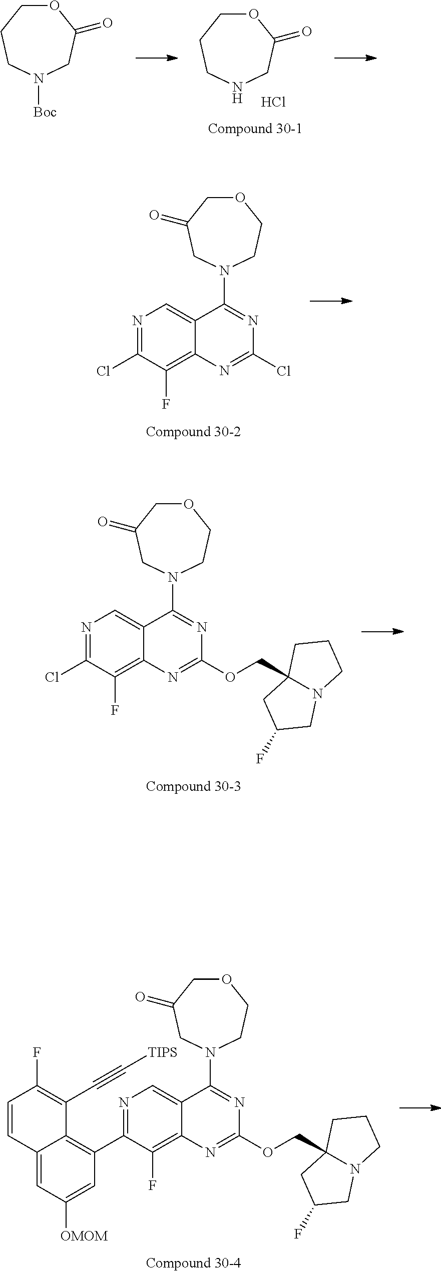

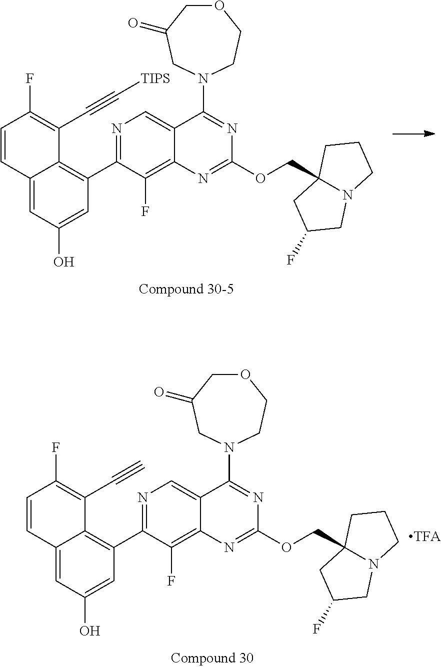

To a solution of tert-butyl 6-oxo-1,4-oxazepane-4-carboxylate (4065 mg, 18.8854 mmol) in DCM (40 mL) was added HCl (4 M in dioxane, 20 mL) and the mixture was stirred at room temperature for 4 h. The mixture was filtered to give Compound 30-1 (2419 mg, 21.0110 mmol, HCl salt). MS: m/z: 116 [M+H]⁺.

A solution of Compound 30-1 (2200 mg, 19.1088 mmol), INT 1 (4600 mg, 18.2207 mmol) and N, N-diisopropylethylamine (5862 mg, 45.3566 mmol) in DCM (100 mL) was stirred at room temperature for 2 h. The solution was diluted with 10% NaHCO₃ solution (100 mL) and extracted with DCM (2×100 mL). The organic layer was washed with NaCl aqueous solution, dried over anhydrous Na₂SO₄ and concentrated in vacuum. The residue was purified by Pre-TLC to give Compound 30-2 (5.64 g, 17.0326 mmol, 93.4795% yield). MS: m/z: 331 [M+H]⁺.

A solution of Compound 30-2 (2830 mg, 8.5465 mmol), INT 2 (1.772 g, 11.1306 mmol) and N,N-diisopropylethylamine (3.282 g, 25.3941 mmol) in 1,4-dioxane (60 mL) was stirred at 80° C. for 16 h under nitrogen atmosphere. The mixture was allowed to cool to room temperature, diluted with saturated NaHCO₃ aqueous solution (80 mL) and extracted with EA (2×100 mL). The organic layer was washed with NaCl aqueous solution, then dried over anhydrous Na₂SO₄ and concentrated in vacuum. The residue was purified by Pre-TLC to give Compound 30-3 (814 mg, 1.7935 mmol, 20.9848% yield). MS: m/z: 454 [M+H]⁺.

To a solution of Compound 30-3 (795 mg, 1.7516 mmol) in toluene (40 mL) was added INT 3 (1.392 g, 2.7159 mmol), cataCXium A Pd G3 (247 mg, 339.1599 μmol), potassium phosphate (1.178 g, 5.5496 mmol) and water (8 mL). The mixture was stirred at 100° C. for 4 hours under nitrogen atmosphere. The mixture was allowed to cool to room temperature, diluted with saturated NaHCO₃ aqueous solution (80 mL) and extracted with EA (2×100 mL). The organic layer was washed with NaCl aqueous solution, then dried over anhydrous Na₂SO₄ and concentrated in vacuum. The residue was purified by Pre-TLC to give Compound 30-4 (615 mg, 764.9424 μmol, 43.6709% yield). MS: m/z: 804 [M+H]⁺.

A solution of Compound 30-4 (101 mg, 125.6247 μmol) and HCl (4 M in dioxane, 1 mL) in DCM (5 mL) was stirred at room temperature for 0.5 h. The solution was diluted with 10% NaHCO₃ solution and extracted with DCM (2×20 mL). The organic layer was washed with NaCl aqueous solution, dried over anhydrous Na₂SO₄ and concentrated in vacuum to give Compound 30-5 (95 mg, 125.0116 μmol, 99.5119% yield). MS: m/z: 760 [M+H]⁺.

To a solution of Compound 30-5 (68 mg, 89.4820 μmol) in DMF (5 mL) was added CsF (20 mg, 131.6626 μmol). The reaction mixture was stirred for 2 hours at room temperature under nitrogen atmosphere. The solution was diluted with saturated NaHCO₃ aqueous solution (20 mL) and extracted with EA (2×30 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 49% B in 38 min at a flow rate of 60 mL/min, 240 nm). The organic phase was concentrated and freeze-dried to give Compound 30 (4 mg, TFA salt). MS: m/z: 604 [M+H]⁺.

### Example 31

### 5-ethynyl-6-fluoro-4-(8-fluoro-4-(6-fluoro-3-methyl-1,4-oxazepan-4-yl)-2-(((2R,7aS)-2-fluorotetrahydro-1H-pyrrolizin-7a(5H)-yl)methoxy)pyrido[4,3-d]pyrimidin-7-yl)naphthalen-2-ol (“Compound 31”)

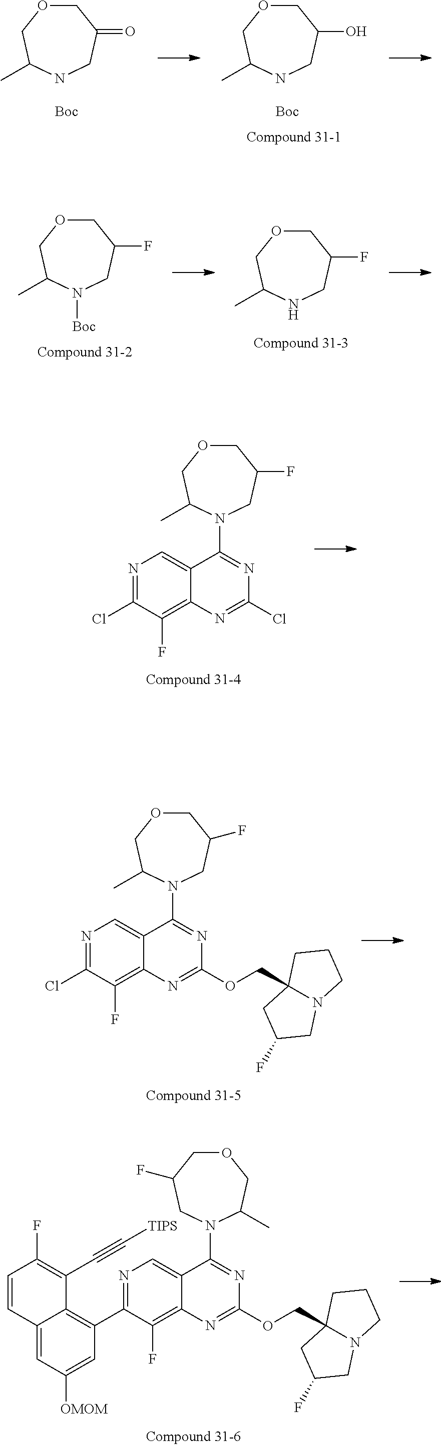

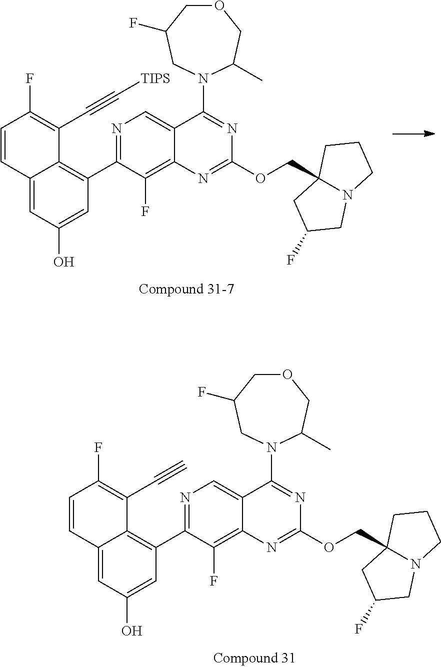

A solution of tert-butyl 3-methyl-6-oxo-1,4-oxazepane-4-carboxylate (300 mg, 1.3085 mmol) in MeOH (6 mL) was added sodium borohydride (64 mg, 1.6917 mmol) at 5° C. The mixture was stirred at room temperature for 2 h. The solution was diluted with saturated solution of ammonium chloride and extracted with EA. The organic layer was washed with NaCl aqueous solution, dried over anhydrous Na₂SO₄ and concentrated in vacuum to give Compound 31-1 (330.5 mg, crude). MS: m/z: 232 [M+H]⁺.

A solution of Compound 31-1 (298 mg, 1.2884 mmol) in DCM (5 mL) was added diethylaminosulfur trifluoride (330 mg, 2.0473 mmol) dropwise at 5° C. and stirred at room temperature for 2 h. The solution was diluted with saturated solution of ammonium chloride and extracted with DCM. The organic layer was washed with NaCl aqueous solution, dried over anhydrous Na₂SO₄ and concentrated in vacuum to give Compound 31-2 (242 mg, 1.0374 mmol, 80.5150% yield). MS: m/z: 234 [M+H]⁺.

To a solution of Compound 31-2 (242 mg, 1.0374 μmol) in DCM (10 mL) was added HCl (4 M in dioxane, 3 mL). The mixture was stirred at room temperature for 2 h. The solution was concentrated in vacuum to give Compound 31-3 which was used for next step directly.

A solution of Compound 31-3, INT 1 (243 mg, 962.5287 μmol) and N,N-diisopropylethylamine (0.4 mL) in DCM (10 mL) was stirred at room temperature for 1 h. The solution was diluted with 10% NaHCO₃ solution (20 mL) and extracted with DCM (2×20 mL). The organic layer was washed with NaCl aqueous solution, dried over anhydrous Na₂SO₄ and concentrated in vacuum. The residue was purified by Pre-TLC to give Compound 31-4 (259.7 mg, 743.7789 μmol, 71.6975% yield). MS: m/z: 349 [M+H]⁺.

A solution of Compound 31-4 (259 mg, 743.7739 μmol), INT 2 (147 mg, 923.3610 μmol) and potassium fluoride (167 mg, 2.8745 mmol) in DMSO (5 mL) was stirred at 94° C. for 16 h under nitrogen atmosphere. The mixture was allowed to cool to room temperature, diluted with saturated NaHCO₃ aqueous solution (20 mL) and extracted with EA (2×20 mL). The organic layer was washed with NaCl aqueous solution, then dried over anhydrous Na₂SO₄ and concentrated in vacuum. The residue was purified by Pre-TLC to give Compound 31-5 (168.3 mg, 356.6413 μmol, 48.0795% yield). MS: m/z: 472 [M+H]⁺.

To a solution of Compound 31-5 (168 mg, 356.0054 μmol) in toluene (5 mL) was added INT 3 (273 mg, 532.6470 μmol), cataCXium A Pd G3 (45 mg, 61.7903 mol), cesium carbonate (389 mg, 1.1939 mmol) and water (1 mL). The reaction mixture was stirred at 100° C. for 16 hours under nitrogen atmosphere. The mixture was allowed to cool to room temperature, diluted with saturated NaHCO₃ aqueous solution (10 mL) and extracted with EA (2×20 mL). The organic layer was washed with NaCl aqueous solution, then dried over anhydrous Na₂SO₄ and concentrated in vacuum. The residue was purified by Pre-TLC to give Compound 31-6 (116.2 mg, 141.3599 μmol, 39.7072% yield). MS: m/z: 822 [M+H]⁺.

A solution of Compound 31-6 (112 mg, 136.2504 μmol) and HCl (4 M in dioxane, 1 mL) in DCM (5 mL) was stirred at room temperature for 0.5 h. The solution was diluted with 10% NaHCO₃ solution and extracted with DCM (2×20 mL). The organic layer was washed with NaCl aqueous solution, dried over anhydrous Na₂SO₄ and concentrated in vacuum to give Compound 31-7 (153 mg, crude). MS: m/z: 778 [M+H]⁺.

To a solution of Compound 31-7 (153 mg, 196.6675 μmol) in DMF (5 mL) was added CsF (177 mg, 1.1652 mmol). The reaction mixture was stirred for 2 hours at 35° C. The solution was diluted with saturated NaHCO₃ aqueous solution (10 mL) and extracted with EA (2×20 mL). The organic layer was dried over anhydrous Na₂SO₄ and concentrated under vacuum. The residue was purified by Prep-HPLC (C18 column, A: 0.1% TFA in water, B: CH₃CN, Gradient: 20% B to 65% B in 60 min at a flow rate of 70 mL/min, 240 nm). The eluent was adjusted to pH=8 and the acetonitrile in the eluent was concentrated. The resulting aqueous phase was extracted with EA and the organic layer was dried, concentrated and freeze-dried to give Compound 31 (31 mg). MS: m/z: 622 [M+H]⁺.

### Example 32

### 5-ethynyl-6-fluoro-4-(8-fluoro-2-(((2S)-2-fluoro-2-methyltetrahydro-1H-pyrrolizin-7a(5H)-yl)methoxy)-4-(1,4-oxazepan-4-yl)pyrido[4,3-d]pyrimidin-7-yl)naphthalen-2-ol (“Compound 32A or Compound 32B”)

### 5-ethynyl-6-fluoro-4-(8-fluoro-2-(((2R)-2-fluoro-2-methyltetrahydro-1H-pyrrolizin-7a(5H)-yl)methoxy)-4-(1,4-oxazepan-4-yl)pyrido[4,3-d]pyrimidin-7-yl)naphthalen-2-ol (“Compound 32B” or “Compound 32A”)

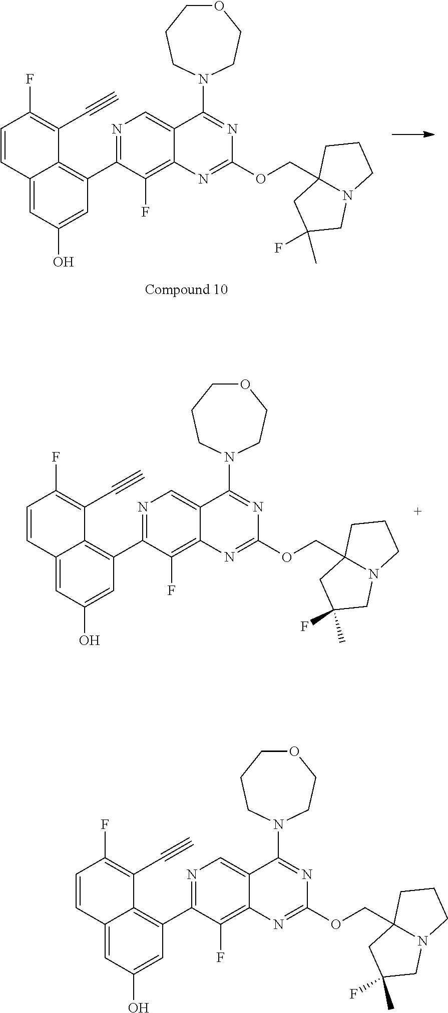 [SMILES: C#Cc1c(F)ccc2cc(O)cc(-c3ncc4c(N5CCCOCC5)nc(OCC56CCCN5CC(C)(F)C6)nc4c3F)c12 ; C#Cc1c(F)ccc2cc(O)cc(-c3ncc4c(N5CCCOCC5)nc(OCC56CCCN5C[C@@](C)(F)C6)nc4c3F)c12 ; C#Cc1c(F)ccc2cc(O)cc(-c3ncc4c(N5CCCOCC5)nc(OCC56CCCN5C[C@](C)(F)C6)nc4c3F)c12]

Compound 10 (8 mg, 13.2531 mol) was separated by Prep-HPLC-Gilson with the following conditions: Column, CHIRALPAK-IG column (2 cm×25 cm, 5 jm); Mobile phase, (hex:DCM=3:1) (0.2% isopropylamine):EtOH=55:45; Flowing rate: 20 ml/min to give Compound 32A (1 mg, the first eluting isomer) and Compound 32B (1 mg, the second eluting isomer) respectively.

### Example 33

### 4-(4-(5-oxa-2-azabicyclo[5.1.0]octan-2-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 33”)

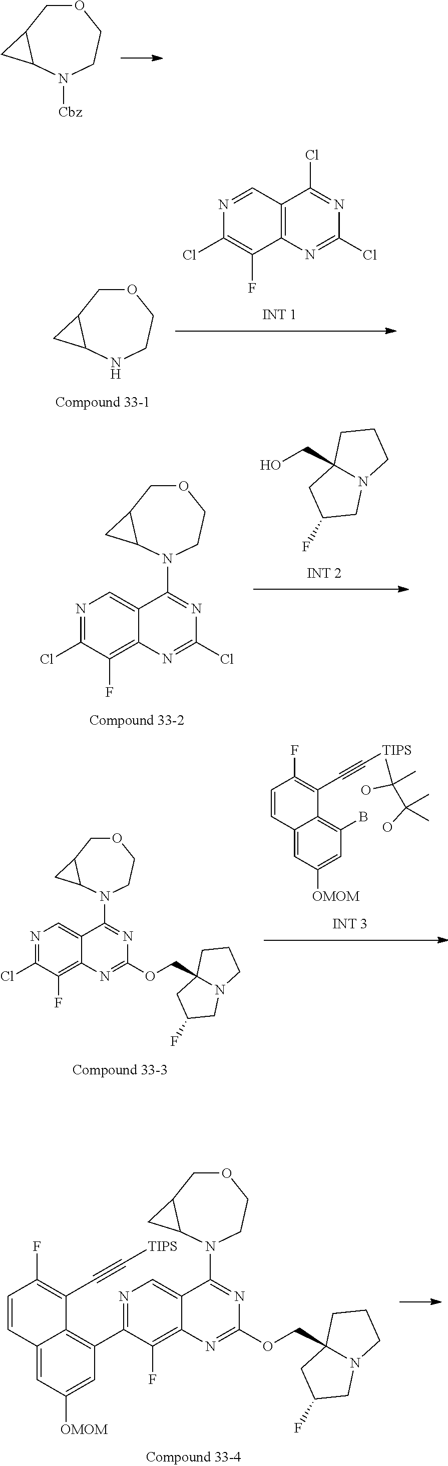

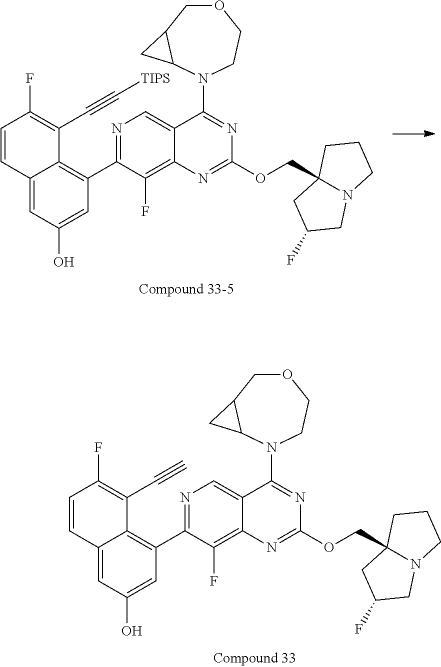

A solution of benzyl 5-oxa-2-azabicyclo[5.1.0]octane-2-carboxylate (118 mg, 477.1745 μmol), di-tert-butyl dicarbonate (303 mg, 1.3883 mmol) and Pd/C (170 mg, 10% Pd content) in EA (5 mL) was stirred at room temperature for 3 h under hydrogen atmosphere. The solution was filtered and concentrated in vacuum. The residue and TFA (1 mL) in DCM (5 mL) was stirred at 0° C. for 3 h. The solution was concentrated in vacuum to give crude Compound 33-1 (54 mg, 477.2122 μmol, 100.0079% yield). MS: m/z: 114 [M+H]⁺.

A solution of INT 1 (153 mg, 606.0366 μmol), Compound 33-1 (54 mg, 477.2122 μmol) and N,N-Diisopropylethylamine (150 mg, 1.1606 mmol) in DCM (10 mL) was stirred at 0° C. for 1 h. The solution was diluted with water (50 mL) and extracted with DCM (2×30 mL). The organic layer was dried over anhydrous Na₂SO₄ and concentrated in vacuum to give crude Compound 33-2 (233 mg, 707.8697 μmol, 116.8031% yield). MS: m/z: 329 [M+H]⁺.

A solution of Compound 33-2 (233 mg, 707.8697 μmol), INT 2 (171 mg, 1.0741 mmol) and KF (132 mg, 2.2721 mmol) in DMSO (15 mL) was stirred at 85° C. for 16 h under nitrogen atmosphere. The mixture was allowed to cool to room temperature, diluted with EA (30 mL), washed with NaCl aqueous solution (20 mL×3), dried over anhydrous Na₂SO₄ and concentrated in vacuum. The residue was purified by Pre-TLC (DCM/MeOH=15:1) to give Compound 33-3 (120 mg, 265.5476 μmol, 37.5137% yield). MS: m/z: 452 [M+H]⁺.

A solution of Compound 33-3 (120 mg, 265.5476 μmol), toluene (10 mL), INT 3 (166 mg, 323.8806 mol), cataCXium A Pd G3 (45 mg, 61.7903 μmol), cesium carbonate (326 mg, 1.0006 mmol) and water (2 mL) was stirred at 100° C. for 16 hours under nitrogen atmosphere. The mixture was allowed to cool to room temperature, diluted with water (50 mL), extracted with DCM (2×30 mL), dried over anhydrous Na₂SO₄ and concentrated in vacuum. The residue was purified by Pre-TLC (DCM/MeOH=15:1) to give Compound 33-4 (153 mg, 190.7709 μmol, 71.8406% yield). MS: m/z: 802 [M+H]⁺.

A solution of Compound 33-4 (153 mg, 190.7709 μmol) and HCl (4 M in dioxane, 1 mL) in DCM (5 mL) was stirred at room temperature for 1 h. The solution was diluted with 10% Na₂CO₃ solution (50 mL), extracted with DCM (2×30 mL), dried over anhydrous Na₂SO₄ and concentrated in vacuum to give crude Compound 33-5 (172 mg, 226.9259 μmol, 118.9520% yield). MS: m/z: 758 [M+H]⁺.

A solution of Compound 33-5 (172 mg, 226.9259 μmol) and CsF (866 mg, 5.7010 mmol) in DMF (10 mL) was stirred for 16 hours at 35° C. under nitrogen atmosphere. The solution was diluted with EA (50 mL), washed with saturated NaCl aqueous solution (50 mL×3), dried over anhydrous Na₂SO₄ and concentrated under vacuum. The residue was purified by Prep-HPLC (C18 column, A: 0.1% TFA in water, B: CH₃CN, Gradient: 15% B to 45% B in 40 min at a flow rate of 60 mL/min, 240 n). The eluent was adjusted to pH=8 and the acetonitrile in the eluent was concentrated. The resulting aqueous phase was extracted with EA and the organic phase was dried, concentrated and freeze-dried to give Compound 33 (46 mg, 76.4606 μmol, 33.6941% yield). MS: m/z: 602 [M+H]⁺.

### Example 34

### 4-(5-(difluoromethyl)-8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)-5-ethynyl-6-fluoronaphthalen-2-ol trifluoroacetic acid (“Compound 34”)

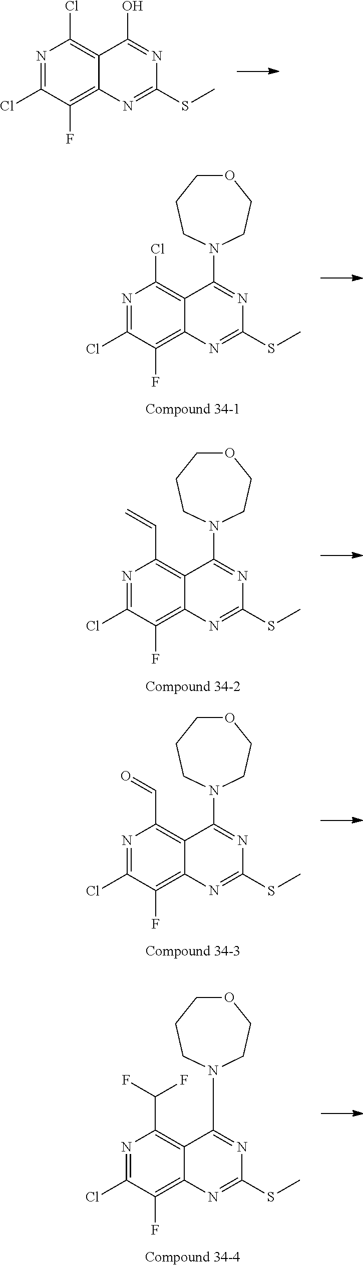 [SMILES: CSc1nc(O)c2c(Cl)nc(Cl)c(F)c2n1 ; CSc1nc(N2CCCOCC2)c2c(Cl)nc(Cl)c(F)c2n1 ; C=Cc1nc(Cl)c(F)c2nc(SC)nc(N3CCCOCC3)c12 ; CSc1nc(N2CCCOCC2)c2c(C=O)nc(Cl)c(F)c2n1 ; CSc1nc(N2CCCOCC2)c2c(C(F)F)nc(Cl)c(F)c2n1]

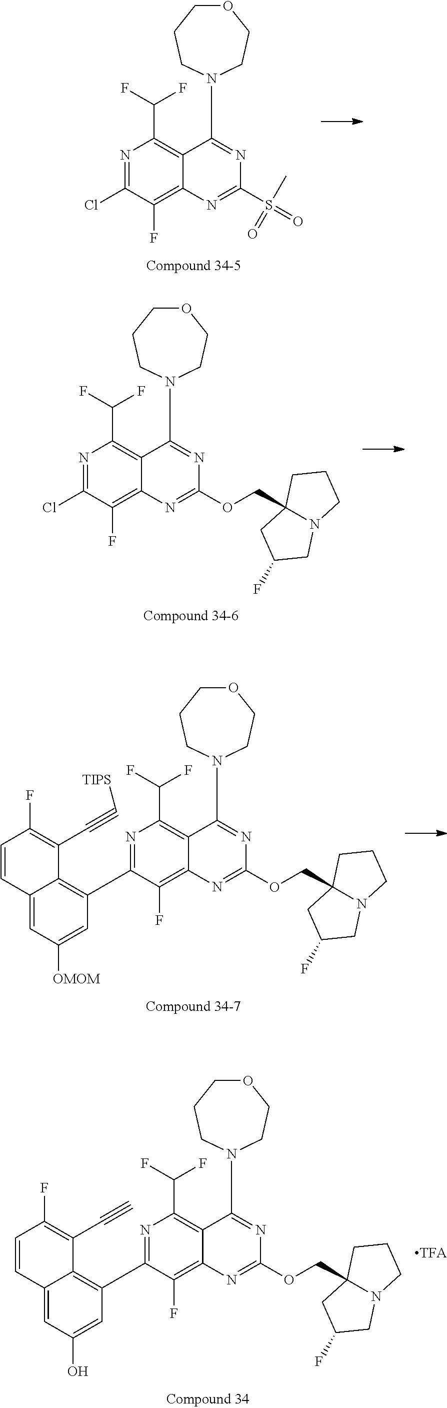

A solution of 5,7-dichloro-8-fluoro-2-(methylthio)pyrido[4,3-d]pyrimidin-4-ol (2505 mg, 8.9430 mmol), phosphorus oxychloride (3461 mg, 22.5719 mmol) and DIEA (1520 mg, 11.7608 mmol) in MeCN (50 mL) was stirred at 85° C. for 3 h. The reaction was concentrated under reduced pressure to give a mixture. To a 0° C. solution of the mixture and DIEA (1.1558 g, 8.9430 mmol) in MeCN (50 mL) was added 1,4-oxazepane (898 mg, 8.8782 mmol). The mixture was stirred at 0° C. for 1 h. The mixture was added water (200 mL), filtered and the solid was collected. The solid was dried to give Compound 34-1 (2669 mg, 7.3478 mmol, 82.1623% yield) MS: m/z 363 [M+H].

A solution of 2-ethenyl-4,4,5,5-tetramethyl-1,3,2-dioxaborolane (525 mg, 3.4088 mmol), Compound 34-1 (1636 mg, 4.5039 mmol), Pd(dppf)₂Cl₂ (1225 mg, 1.6742 mmol) and K₃PO₄ (1882 mg, 8.8662 mmol) in toluene (20 mL) and water (2 mL) was stirred at 100° C. overnight under protected nitrogen. The mixture was added water (10 mL) and extracted with EA (10 mL×3). The organic phases were combined, washed with saturated sodium chloride solution (10 mL×3) and dried over anhydrous sodium sulfate. The organic phase was filtrated, concentrated and purified by pre-TLC to give Compound 34-2 (982 mg, 2.7675 mmol, 61.4468% yield) MS: m/z 355 [M+H].

A solution of Compound 34-2 (358 mg, 1.0089 mmol), potassium osmate(VI) dehydrate (168 mg, 455.9585 μmol), sodium periodate, (1575 mg, 7.3636 mmol) and 2,6-lutidine (405 mg, 3.7797 mmol) in 1,4-dioxane (15 mL) and water (5 mL) was stirred at room temperature overnight. The mixture was added water (10 mL) and extracted with EA (10 mL×3). The organic phases were combined, washed with saturated sodium chloride solution (10 mL×3) and dried over anhydrous sodium sulfate. The organic phase was filtrated, concentrated and purified by pre-TLC to give Compound 34-3 (261 mg, 731.4973 μmol, 72.5018% yield) MS: m/z 357 [M+H].

A solution of Compound 34-3 (52 mg, 145.7391 μmol) and DAST (104 mg, 645.2065 μmol) in DCM (10 mL) was stirred at room temperature overnight. The mixture was quenched with saturated NaHCO₃ aqueous solution (20 mL) and extracted with DCM (10 mL×2). The combined organic extracts were washed with brine (10 mL×3) and dried over anhydrous Na₂SO₄. The mixture was concentrated under reduced pressure. The mixture was purified by pre-TLC to give Compound 34-4 (26 mg, 68.6378 μmol, 47.0964% yield) MS: m/z 379 [M+H].

A solution of Compound 34-4 (26 mg, 68.6378 μmol) and m-CPBA (32 mg, 185.4363 μmol) in DCM (10 mL) was stirred at room temperature for 2 h. The mixture was quenched with saturated Na₂S₂O₃ aqueous solution (10 mL) and extracted with DCM (10 mL×2). The combined organic extracts were washed with brine (10 mL×3) and dried over anhydrous Na₂SO₄. The mixture was concentrated under reduced pressure to give Compound 34-5 (95 mg, 231.2569 μmol, 336.9235% yield) MS: m/z 411 [M+H]⁺.

A solution of INT 2 (126 mg, 791.4538 μmol), Compound 34-5 (95 mg, 231.2569 μmol) and DIEA (217 mg, 1.6790 mmol) in 1,4-dioxane was stirred at 100° C. overnight. The reaction mixture was concentrated and purified by pre-TLC to give Compound 34-6 (43 mg, 87.7742 μmol, 37.9553% yield) MS: m/z 490 [M+H]⁺.

A solution of INT 3 (124 mg, 241.9349 μmol), Compound 34-6 (43 mg, 87.7743 μmol), cataCXium A Pd G3 (36 mg, 49.4322 μmol) and Cs₂CO₃ (190 mg, 583.1460 μmol) in toluene (5 mL) and water (1 mL) was stirred at 100° C. overnight under protected nitrogen. The mixture was added water (10 mL) and extracted with EA (10 mL×3). The organic phases were combined, washed with saturated sodium chloride solution (10 mL×3) and dried over anhydrous sodium sulfate. The organic phase was filtrated, concentrated and purified by pre-HPLC to give Compound 34-7 (147 mg, 174.9988 μmol, 199.3736% yield) MS: m/z 840 [M+H].

To a solution of Compound 34-7 (147 mg, 174.9988 mol) in MeCN (5 mL) was added hydrochloric acid solutions (1 mL). The mixture was stirred at room temperature for 2 h. The residue was added TEA to adjust pH to about 8. The mixture was filtered and the organic phase was collected. The organic phase was concentrated under reduced pressure to give a mixture. To a solution of the mixture in DMF (5 mL) was added CsF (331 mg, 2.1790 mmol). The reaction mixture was stirred at 45° C. for 2 h. The mixture was filtered and the organic phase was collected. The residue was purified by pre-HPLC (C18 column, phase A: 0.1% TFA in water, phase B: CH₃CN, Gradient: 15% B to 30% B in 40 min at a flow rate of 60 mL/min, 230 nm) and lyophilized to give Compound 34 (0.0031 g, 4.8467 μmol, 2.7695% yield, TFA salt) MS: m/z 640 [M+H]⁺.

### Example 35

### 4-(5-cyclopropyl-8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)-5-ethynyl-6-fluoronaphthalen-2-ol trifluoroacetic acid

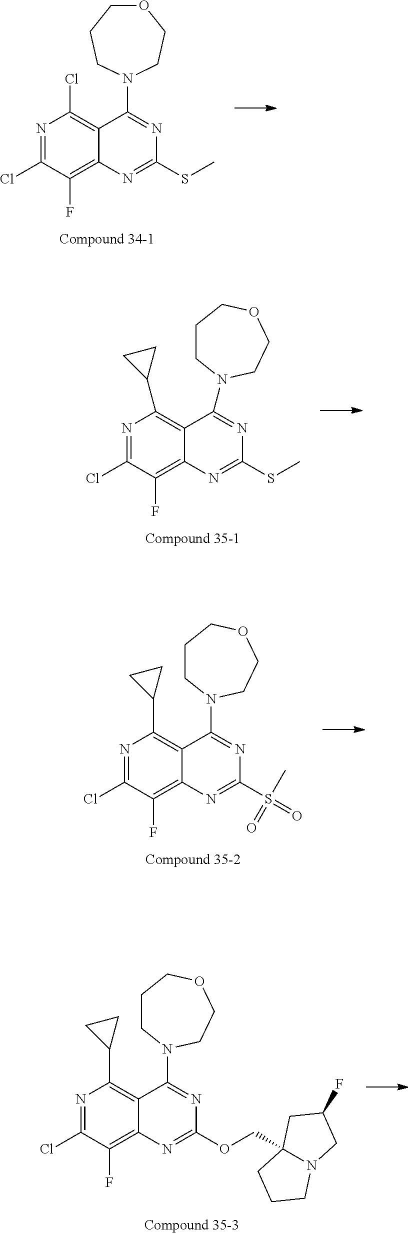 [SMILES: CSc1nc(N2CCCOCC2)c2c(Cl)nc(Cl)c(F)c2n1 ; CSc1nc(N2CCCOCC2)c2c(C3CC3)nc(Cl)c(F)c2n1 ; CS(=O)(=O)c1nc(N2CCCOCC2)c2c(C3CC3)nc(Cl)c(F)c2n1 ; Fc1c(Cl)nc(C2CC2)c2c(N3CCCOCC3)nc(OC[C@@]34CCCN3C[C@H](F)C4)nc12]

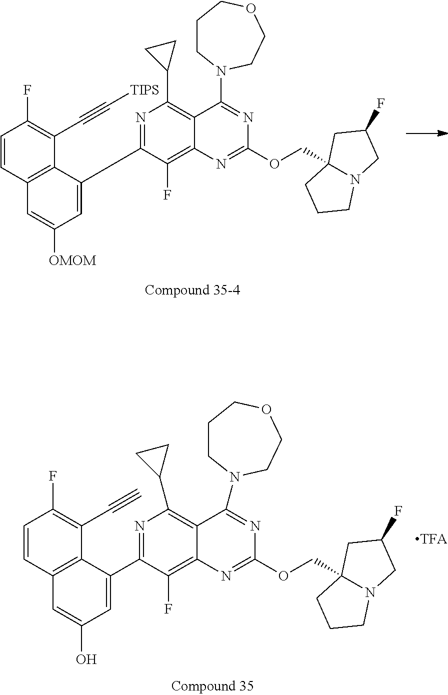

A solution of Compound 34-1 (504 mg, 1.3875 mmol), cyclopropylboronic acid (188 mg, 2.1887 mmol), potassium phosphate (882 mg, 4.1552 mmol), PdCl₂(dppf)₂ (234 mg, 319.8016 μmol) in toluene (10 mL) and water (1 mL) was stirred at 100° C. overnight under protected nitrogen. The mixture was added water (10 mL) and extracted with EA (10 mL×3). The organic phases were combined, washed with saturated sodium chloride solution (10 mL×3) and dried over anhydrous sodium sulfate. The organic phase was filtrated, concentrated and purified by pre-TLC to give Compound 35-1 (191 mg, 517.8173 μmol, 37.3196% yield) MS: m/z 369 [M+H]1.

A solution of Compound 35-1 (148 mg, 401.2406 μmol) and m-CPBA (192 mg, 1.1126 mmol) in DCM (10 mL) was stirred at room temperature for 2 h. The mixture was quenched with saturated Na₂S₂O₃ aqueous solution (10 mL) and extracted with DCM (10 mL×2). The combined organic extracts were washed with brine (10 mL×3) and dried over anhydrous Na₂SO₄. The mixture was concentrated under reduced pressure to give Compound 35-2 (435 mg, 1.0852 mmol, 270.4564% yield). MS: m/z 401 [M+H]⁺.

A solution of Compound 35-2 (404 mg, 1.0953 mmol), INT 2 (652 mg, 4.0955) and DIEA (778 mg, 6.0197 mmol) in 1,4-dioxane (5 mL) was stirred at 100° C. overnight. The residue was purified by pre-HPLC to give Compound 35-3 (134 mg, 279.1961 μmol, 25.4909% yield). MS: m/z 480 [M+H]⁺.

A solution of INT 3 (349 mg, 680.9296 μmol), Compound 35-3 (134 mg, 279.1963 μmol), cataCXium A Pd G3 (92 mg, 126.3268 μmol) and Cs₂CO₃ (275 mg, 844.0272 μmol) in toluene (10 mL) and water (2 mL) was stirred at 100° C. overnight under protected nitrogen. The mixture was added water (10 mL) and extracted with EA (10 mL×3). The organic phases were combined, washed with saturated sodium chloride solution (10 mL×3) and dried over anhydrous sodium sulfate. The organic phase was filtrated, concentrated and purified by pre-HPLC to give Compound 35-4 (282 mg, 339.7336 μmol, 121.6827% yield). MS: m/z 830[M+H]⁺.

To a solution of Compound 35-4 (282 mg, 339.7337 μmol) in MeCN (5 mL) was added hydrochloric acid solutions (1 mL). The mixture was stirred at room temperature for 2 h. The residue was added TEA to adjust pH to about 8. The mixture was filtered and the organic phase was collected. The organic phase was concentrated under reduced pressure to give a mixture. To a solution of the mixture in DMF (5 mL) was added CsF (1372 mg, 9.0321 mmol). The reaction mixture was stirred at 45° C. for 2 h. The mixture was filtered and the organic phase was collected. The residue was purified by pre-HPLC (C18 column, phase A: 0.1% TFA in water, phase B: CH₃CN, Gradient: 15% B to 30% B in 40 min at a flow rate of 60 mL/min, 230 nm) and lyophilized to give Compound 35 (0.0437 g, 69.4014 μmol, 20.4282% yield, TFA salt). MS: m/z 630[M+H]⁺.

### Example 36

### 5-ethynyl-6-fluoro-4-(8-fluoro-2-(((2R,7aS)-2-fluorotetrahydro-1H-pyrrolizin-7a(5H)-yl)methoxy)-5-(hydroxymethyl)-4-(1,4-oxazepan-4-yl)pyrido[4,3-d]pyrimidin-7-yl)naphthalen-2-ol trifluoroacetic acid (“Compound 36”)

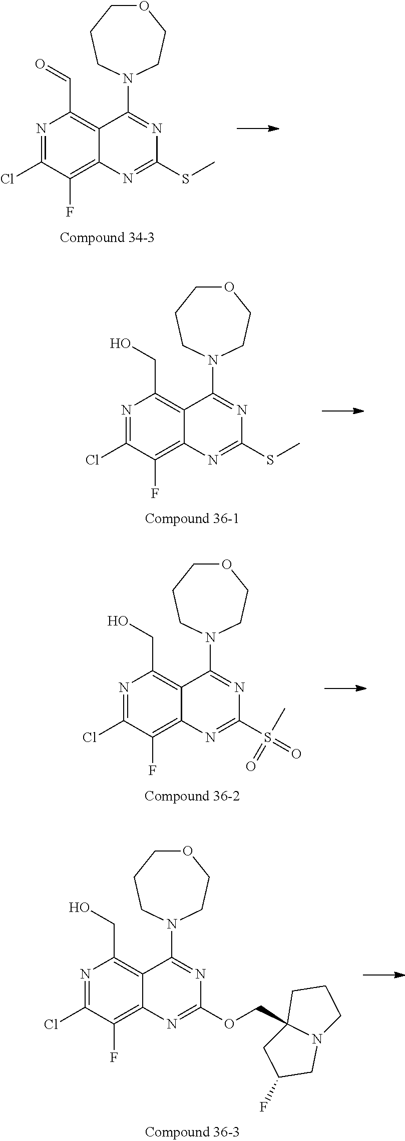 [SMILES: CSc1nc(N2CCCOCC2)c2c(C=O)nc(Cl)c(F)c2n1 ; CSc1nc(N2CCCOCC2)c2c(CO)nc(Cl)c(F)c2n1 ; CS(=O)(=O)c1nc(N2CCCOCC2)c2c(CO)nc(Cl)c(F)c2n1 ; OCc1nc(Cl)c(F)c2nc(OC[C@@]34CCCN3C[C@H](F)C4)nc(N3CCCOCC3)c12]

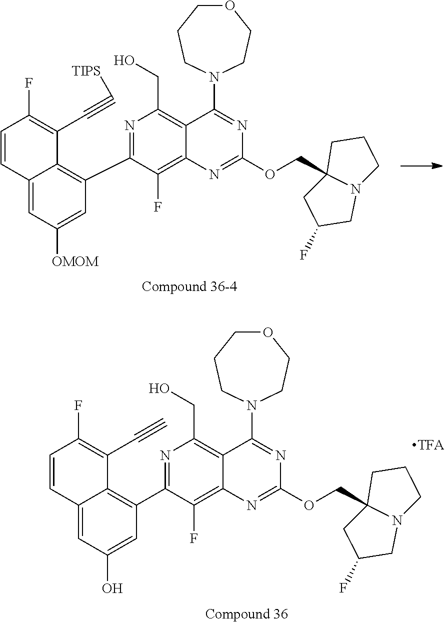

To a 0° C. solution of Compound 34-3 (73 mg, 204.5953 μmol) in MeOH (5 mL) was added NaBH₄ (46 mg, 1.2159 mmol). The mixture was stirred at 0° C. for 2 h. The mixture was quenched with ice water (5 mL) and extracted with EA (10 mL×3). the organic phases were combined, washed with saturated sodium chloride solution (10 mL×3) and dried over anhydrous sodium sulfate. The organic phase was filtrated, concentrated and purified by pre-TLC to give Compound 36-1 (43 mg, 119.8378 μmol, 58.5731% yield). MS: m/z 359 [M+H]⁺.

A solution of Compound 36-1 (43 mg, 119.8379 μmol) and m-CPBA (51 mg, 295.5391 μmol) in DCM (5 mL) was stirred room temperature for 2 h. The mixture was quenched with saturated Na₂S₂O₃ aqueous solution (10 mL) and extracted with DCM (10 mL×2). The combined organic extracts were washed with brine (10 mL×3) and dried over anhydrous Na₂SO₄. The mixture was concentrated under reduced pressure to give Compound 36-2 (62 mg, 158.6420 μmol, 132.3805% yield). MS: m/z 391 [M+H].

A solution of INT 2 (108 mg, 678.3877 μmol), Compound 36-2 (62 mg, 158.6420 μmol) and DIEA (173 mg, 1.3386 mmol) in 1,4-dioxane (3 mL) was stirred at 100° C. overnight. The mixture was concentrated and purified by pre-HPLC to give Compound 36-3 (46 mg, 97.8908 μmol, 61.7054% yield). MS: m/z 470 [M+H]⁺.

A solution of Compound 36-3 (46 mg, 97.8907 μmol), INT 3 (134 mg, 261.4456 μmol), cataCXium A Pd G3 (43 mg, 59.0440 μmol) and Cs₂CO₃ (128 mg, 392.8563 μmol) in toluene (8 mL) and water (2 mL) was stirred at 100° C. overnight under protected nitrogen. The mixture was added water (10 mL) and extracted with EA (10 mL×3). The organic phases were combined, washed with saturated sodium chloride solution (10 mL×3) and dried over anhydrous sodium sulfate. The organic phase was filtrated, concentrated and purified by pre-HPLC to give Compound 36-4 (79 mg, 96.3386 μmol, 98.4145% yield). MS: m/z 820 [M+H].

To a solution of Compound 36-4 (0.079 g, 96.3386 μmol) in MeCN (5 mL) was added hydrochloric acid solutions (1 mL). The mixture was stirred at room temperature for 2 h. The residue was added TEA to adjust pH to about 8. The mixture was filtered and the organic phase was collected. The organic phase was concentrated under reduced pressure to give a mixture. To a solution of the mixture in DMF (5 mL) was added CsF (558 mg, 3.6734 mmol). The reaction mixture was stirred at 45° C. for 2 h. The mixture was filtered and the organic phase was collected. The residue was purified by pre-HPLC (C18 column, phase A: 0.1% TFA in water, phase B: CH₃CN, Gradient: 15% B to 30% B in 40 min at a flow rate of 60 mL/min, 230 nm) and lyophilized to give Compound 36 (0.0027 g, 4.3574 μmol, 4.5230% yield, TFA salt). MS: m/z 620 [M+H]⁺.

### Example 37

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)naphthalen-2-ol (“Compound 37”)

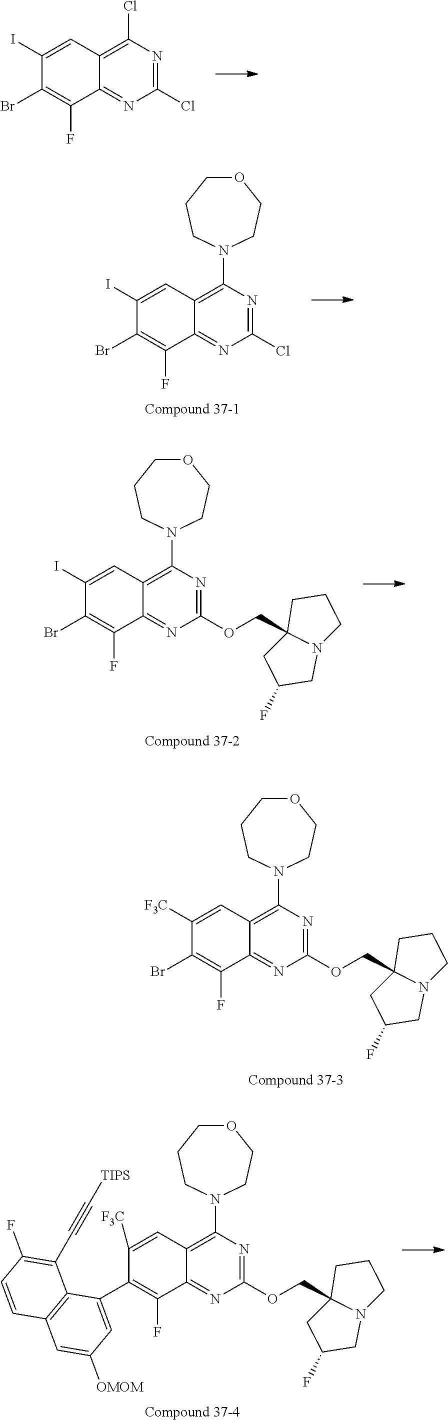

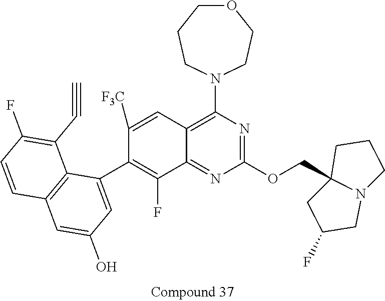 [SMILES: C#Cc1c(F)ccc2cc(O)cc(-c3c(C(F)(F)F)cc4c(N5CCCOCC5)nc(OC[C@@]56CCCN5C[C@H](F)C6)nc4c3F)c12]

To a solution of 7-bromo-2,4-dichloro-8-fluoro-6-iodoquinazoline (5.10 g, 12.09 mmol) and DIEA (4.62 g, 35.75 mmol) in DCM (50 mL) was added 1,4-oxazepane (1.06 g, 10.48 mmol) portion-wise at 0° C. The mixture was stirred at room temperature for 1 hour. The reaction mixture was diluted with DCM (20 mL), washed with water (2×20 mL), dried over anhydrous Na₂SO₄ and concentrated under reduced pressure. The residue was triturated with Hex:EtOAc (15:1, v/v, 200 mL) to give Compound 37-1 (4.79 g, 9.85 mmol, 81.43% yield). MS m/z: 486/488 [M+H]⁺.

To a solution of Compound 37-1 (2.56 g, 5.26 mmol) and INT 2 (967 mg, 6.07 mmol) in DMF/THF (40 mL, v/v=1:1) was added triethylenediamine (356 mg, 3.17 mmol) and Cs₂CO₃ (2.59 g, 7.95 mmol). The mixture was stirred at room temperature overnight under nitrogen atmosphere. The reaction mixture was extracted with EtOAc (50 mL) and washed with brine (2×30 mL), then dried over anhydrous Na₂SO₄ and concentrated under reduced pressure. The residue was purified by silica gel chromatography (eluted with DCM:MeOH=50:1 to 30:1, v/v) to give Compound 37-2 (2.70 g, 4.08 mmol, 77.48% yield) as. MS m/z: 609/611 [M+H]⁺.

To a solution of Compound 37-2 (1.02 g, 1.68 mmol), diphenyl-(trifluoromethyl)-sulfonium trifluoromethanesulfonate (0.96 g, 2.37 mmol) in NMP (15 mL) was added copper powder (325 mg, 5.11 mmol). The reaction mixture was stirred at 58° C. overnight under nitrogen atmosphere. The mixture was extracted with EtOAc (50 mL), washed with brine (2×50 mL), dried over anhydrous Na₂SO₄ and concentrated under reduced pressure. The residue was purified by reversed phase flash (0.1% TFA in water:CH₃CN=15% to 45%) to give Compound 37-3 (335 mg, 0.608 mmol, 36.19% yield). MS m/z: 551/553 [M+H]⁺.

To a solution of Compound 37-3 (106 mg, 0.174 mmol), INT 3 (152 mg, 0.297 mmol) in toluene (4 mL) and water (1 mL) were added Cs₂CO₃ (191 mg, 0.586 mmol) and cataCXium A Pd G₃ (22 mg, 0.030 mmol). The reaction mixture was stirred at 100° C. overnight under nitrogen atmosphere. The mixture was filtered and the filtrate was concentrated under reduced pressure. The residue was purified by Pre-TLC (eluted with DCM:MeOH=20:1, v/v) to give Compound 37-4 (50 mg, 0.058 mmol, 33.53% yield). MS m/z: 857 [M+H]⁺.

A solution of Compound 37-4 (50 mg, 0.058 mmol) and HCl (1 mL, 4 M in dioxane) in MeCN (3 mL) was stirred at room temperature for 1 hour. The mixture was concentrated under reduced pressure. The residue was diluted with saturated NaHCO₃ aqueous solution (20 mL) and extracted with EtOAc (2×20 mL). The organic layers were combined, dried over anhydrous Na₂SO₄ and concentrated under reduced pressure. To the residue in DMF (4 mL) was added CsF (180 mg, 1.19 mmol). The reaction mixture was stirred at 45° C. for 2 hours under nitrogen atmosphere. The reaction mixture was purified by Prep-HPLC (C18 column, A: 0.1% NH₄OH in water, B: CH₃CN, Gradient: 30% B to 60% B in 60 min at a flow rate of 40 mL/min, 236 nm) and freeze-dried to give Compound 37 (7.8 mg, 0.012 mmol). MS m/z: 657 [M+H]⁺.

### Example 38

### 5-ethynyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)-6-methylnaphthalen-2-ol trifluoroacetic acid (“Compound 38”)

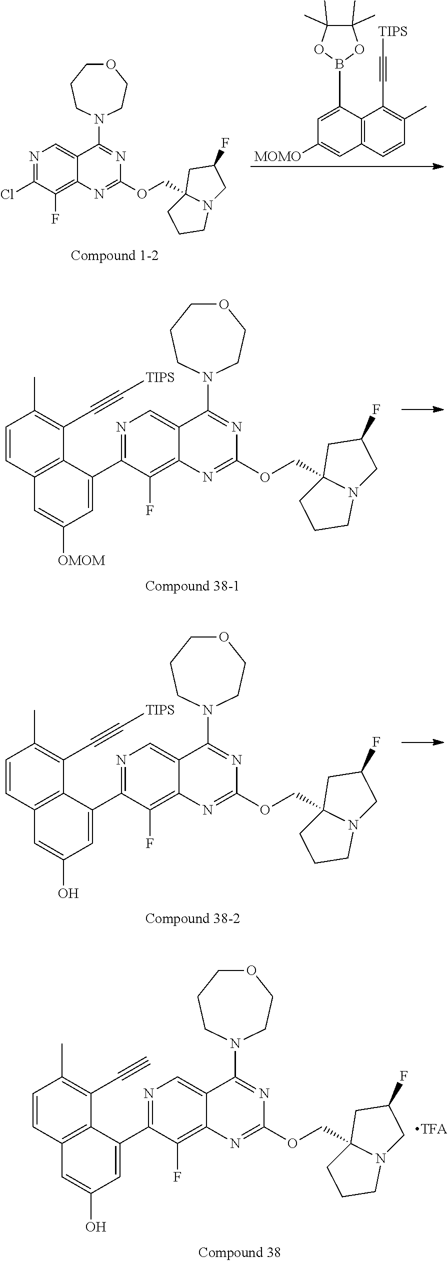

To a solution of Compound 1-2 (74 mg, 168.22 μmol) and triisopropyl((6-(methoxymethoxy)-2-methyl-8-(4,4,5,5-tetramethyl-1,3,2-dioxaborolan-2-yl)naphthalen-1-yl)ethynyl)silane (100 mg, 196.62 gmol) in toluene (4 mL) and water (1 mL) were added Cs₂CO₃ (139 mg, 426.61 gmol) and cataCXium A Pd G₃ (21 mg, 28.83 gmol). The reaction mixture was purged with nitrogen for three times and stirred at 100° C. for 16 hours. After cooling to room temperature, the mixture was diluted with EtOAc (50 mL) and washed with water (3×30 mL). The organic layer was dried over anhydrous Na₂SO₄ and concentrated under reduced pressure. The residue was purified by Pre-TLC (eluted with DCM:MeOH=15:1, v/v) to give Compound 38-1 (73 mg, 92.87 μmol, 55.2% yield). MS (ESI, m/z): 786 [M+H]⁺.

To a solution of Compound 38-1 (92 mg, 117.04 μmol) in CH₃CN (6 mL) was added HCl (4 M in 1,4-dioxane, 2 mL). The reaction mixture was stirred at room temperature for 1 hour. The resulting mixture was concentrated under reduced pressure. The residue was dissolved in EtOAc (50 mL), washed with saturated NaHCO₃ aqueous solution (3×30 mL). The organic layer was dried over anhydrous Na₂SO₄ and concentrated under reduced pressure to give Compound 38-2 (92 mg, 123.99 μmol, 105.9% yield). MS (ESI, m/z): 742 [M+H]⁺.

To a solution of Compound 38-2 (92 mg, 123.99 μmol) in DMF (3 mL) was added CsF (0.53 g, 3.48 mmol). The reaction mixture was stirred at room temperature for 3 hours then filtered to collect the filtrate. The filtrate was concentrated under reduced pressure. The residue was purified by Prep-HPLC (Agela Venusil PrepG C18, phase A: 0.1% TFA in water, phase B: CH₃CN, Gradient: 15% B to 45% B in 37 min at a flow rate of 60 mL/min, 235 nm) and freeze-dried to give Compound 38 (52.5 mg, 75.03 μmol, TFA salt, 60.5% yield). MS (ESI, m/z): 586 [M+H]⁺.

### Example 39

### 6-chloro-5-ethynyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol trifluoroacetic acid (“Compound 39”)

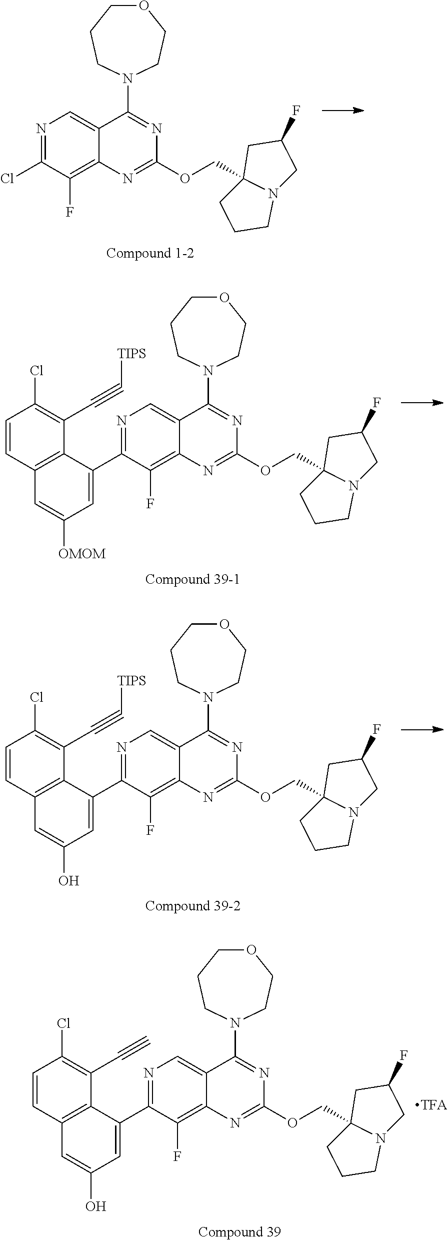

To a solution of Compound 1-2 (101 mg, 0.23 mmol), ((2-chloro-6-(methoxymethoxy)-8-(4,4,5,5-tetramethyl-1,3,2-dioxaborolan-2-yl)naphthalen-1-yl)ethynyl)triisopropylsilane (151 mg, 0.29 mmol) in toluene (4 mL) and water (1 mL) were added Cs₂CO₃ (156 mg, 0.48 mmol) and cataCXium A Pd G₃ (34 mg, 0.047 mmol). The reaction mixture was stirred at 100° C. overnight under nitrogen atmosphere. The mixture was allowed to cool to room temperature, then diluted with water (30 mL) and extracted with EtOAc (20 mL). The organic layer was washed with NaCl aqueous solution (30 mL), dried over anhydrous Na₂SO₄ and concentrated under reduced pressure. The residue was purified by Pre-TLC to give Compound 39-1 (60 mg, 74.40 μmol). MS m/z: 806 [M+H]⁺.

A solution of Compound 39-1 (60 mg, 74.40 μmol) and HCl (4 M in dioxane, 1 mL) in CH₃CN (3 mL) was stirred at room temperature for 1 hour. The solution was concentrated under reduced pressure, diluted with saturated NaHCO₃ aqueous solution (20 mL) and extracted with EtOAc (30 mL×2). The collected organic layer was washed with brine (30 mL), dried over anhydrous Na₂SO₄ and concentrated under reduced pressure to give Compound 39-2 (crude, 54 mg, 70.83 μmol). MS m/z: 762 [M+H]⁺.

A solution of Compound 39-2 (54 mg, 70.83 μmol) and CsF (0.55 g, 3.62 mmol) in DMF (4 mL) was stirred at 45° C. for 3 hours. The mixture was diluted with water (30 mL) and extracted with EtOAc (30 mL). The organic layer was dried over anhydrous Na₂SO₄ and concentrated under reduced pressure. The residue was purified by Prep-HPLC (C18 column, A: 0.1% TFA in water, B: CH₃CN, Gradient: 15% B to 45% B in 42 min at a flow rate of 60 mL/min, 235 nm) and freeze-dried to give Compound 39 (20.4 mg, 28.33 μmol, TFA salt). MS m/z: 606 [M+H]⁺.

### Example 40

### 5,6-difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol trifluoroacetic acid (“Compound 40”)

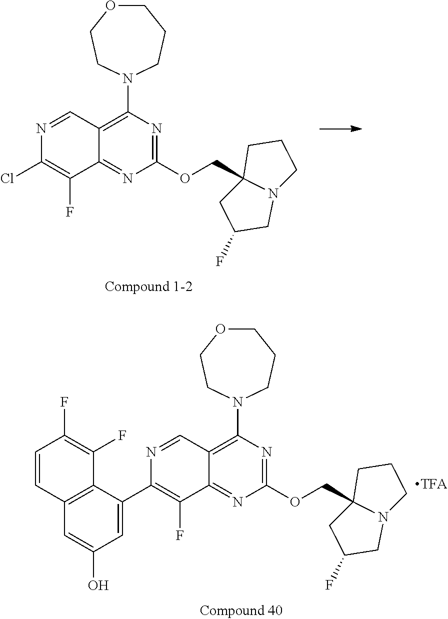 [SMILES: Fc1c(Cl)ncc2c(N3CCCOCC3)nc(OC[C@@]34CCCN3C[C@H](F)C4)nc12 ; Oc1cc(-c2ncc3c(N4CCCOCC4)nc(OC[C@@]45CCCN4C[C@H](F)C5)nc3c2F)c2c(F)c(F)ccc2c1]

To a solution of Compound 1-2 (162 mg, 0.37 mmol), 5,6-difluoro-4-(4,4,5,5-tetramethyl-1,3,2-dioxaborolan-2-yl)naphthalen-2-ol (138 mg, 0.45 mmol) in toluene (6 mL) and water (1.5 mL) were added Cs₂CO₃ (358 mg, 1.10 mmol) and cataCXium A Pd G₃ (38 mg, 0.052 mmol). The reaction mixture was stirred at 100° C. overnight under nitrogen atmosphere. The reaction mixture was diluted with water (30 mL) and extracted EtOAc (2×30 mL). The combined organic layer was dried over anhydrous Na₂SO₄, filtered and concentrated under reduced pressure. The residue was purified by Pre-HPLC (Agela Venusil Prep G C18, 50 mm×250 mm, 10 μm column, A: 0.1% TFA in water, B: CH₃CN, Gradient: 15% B to 45% B in 35 min at a flow rate of 60 mL/min, 240 nm) to give Compound 40 (87.3 mg, 0.13 mmol, 33.98% yield, TFA salt). MS m/z: 584 [M+H]⁺.

### Example 41

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)-6-((trifluoromethyl)thio)quinazolin-7-yl)naphthalen-2-ol trifluoroacetic acid (“Compound 41”)

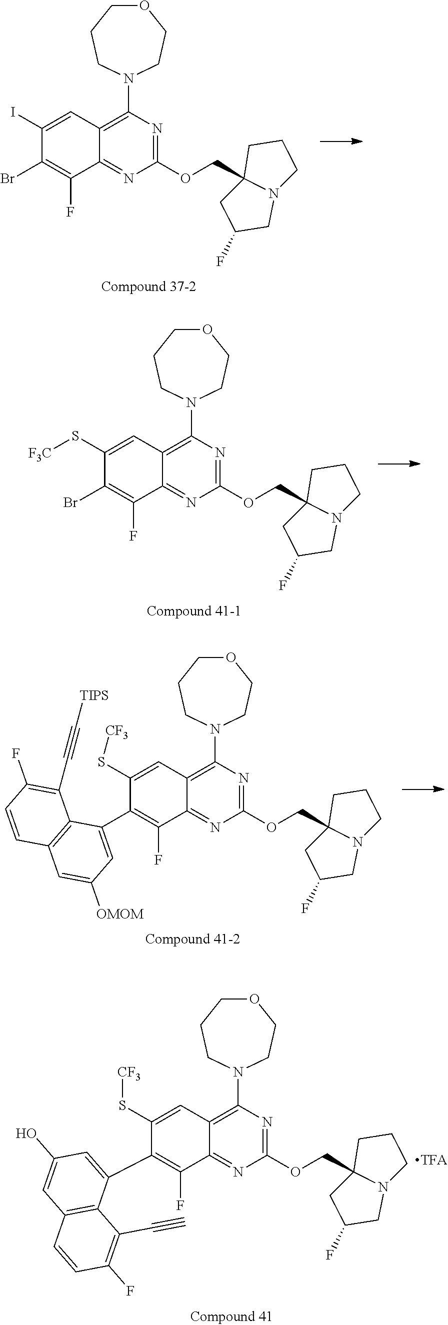

To a solution of Compound 37-2 (504 mg, 0.827 mmol) and (trifluoromethylthio) silver (I) (534 mg, 2.56 mmol) in DMF (10 mL) was added CuI (164 mg, 0.861 mmol). The reaction mixture was stirred at 96° C. overnight under nitrogen atmosphere. The mixture was allowed to cool to room temperature, then diluted with water and extracted with EtOAc (30 mL). The organic layer was washed with brine (2×30 mL), dried over anhydrous Na₂SO₄ and concentrated under reduced pressure. The residue was purified by Pre-TLC (eluted with DCM:MeOH=14:1, v/v) to give Compound 41-1 (120 mg, 0.206 mmol, 24.86% yield). MS m/z: 583/585 [M+H]⁺.

To a solution of Compound 41-1 (84 mg, 0.144 mmol), INT 3 (109 mg, 0.213 mmol) in toluene (4 mL) and water (0.8 mL) were added Cs₂CO₃ (136 mg, 0.417 mmol) and cataCXium A Pd G₃ (11 mg, 0.015 mmol). The reaction mixture was stirred at 100° C. overnight under nitrogen atmosphere. The reaction mixture was filtered and the filtrate was concentrated under reduced pressure. The residue was purified by Pre-TLC (eluted with DCM:MeOH=15:1, v/v) to give Compound 41-2 (80 mg, 0.090 mmol, 62.50% yield). MS m/z: 889 [M+H]⁺.

A solution of Compound 41-2 (80 mg, 0.090 mmol) and HCl (0.8 mL, 4 M in dioxane) in MeCN (3 mL) was stirred at room temperature for 1 hour. The mixture was concentrated under reduced pressure. The residue was diluted with saturated NaHCO₃ aqueous solution (20 mL) and extracted with EtOAc (2×20 mL). The organic layers were combined, dried over anhydrous Na₂SO₄ and concentrated under reduced pressure. The residue in DMF (4 mL) was added CsF (250 mg, 1.65 mmol). The reaction mixture was stirred at 45° C. for 2 hours under nitrogen atmosphere. The reaction mixture was purified by Prep-HPLC (C18 column, A: 0.1% TFA in water, B: CH₃CN, Gradient: 15% B to 60% B in 60 min at a flow rate of 70 mL/min, 236 nm) and freeze-dried to give Compound 41 (30.2 mg, 0.038 mmol, TFA salt). MS m/z: 689 [M+H]⁺.

### Example 42

### 4-(4-(3-oxa-7-azabicyclo[4.1.1]octan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol trifluoroacetic acid (“Compound 42”)

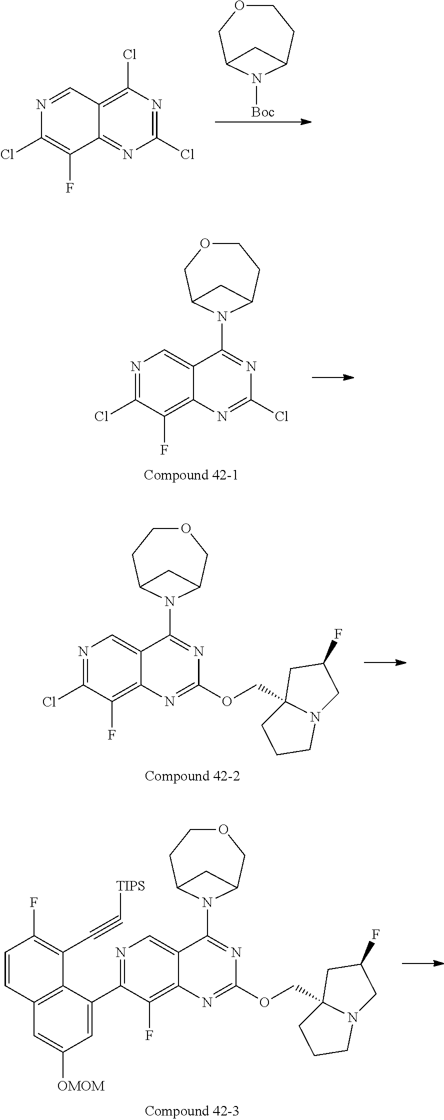

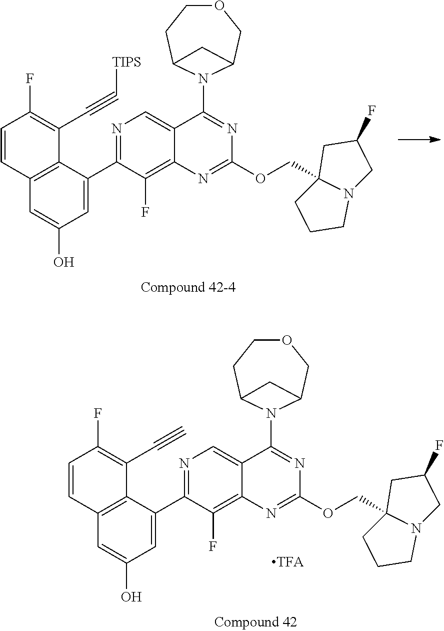

To a solution of tert-butyl 3-oxa-7-azabicyclo[4.1.1]octane-7-carboxylate (140 mg, 656.43 μmol) in CH₃CN (5 mL) was added HCl (4 M in 1,4-dioxane, 1 mL). The reaction mixture was stirred at room temperature for 1 hour. The resulting mixture was concentrated under reduced pressure. The residue was dissolved in DCM (10 mL), N,N-diisopropylethylamine (0.2 mL, 1.21 mmol) and INT 1 (65 mg, 257.46 gmol) were added. The reaction mixture was stirred for 2 hours at room temperature. The mixture was concentrated under reduced pressure and the residue was diluted with EtOAc (50 mL) and washed with water (2×30 mL) and brine (30 mL). The organic layer was dried over anhydrous Na₂SO₄ and concentrated under reduced pressure. The residue was purified by Pre-TLC (Hex:EA=2:1, v/v) to give Compound 42-1 (60 mg, 182.28 μmol, 70.8% yield). MS (ESI, m/z): 329 [M+H]⁺.

To a solution of Compound 42-1 (60 mg, 182.28 μmol) and INT 2 (71 mg, 445.97 μmol) in DMSO (4 mL) was added KF (36 mg, 619.65 μmol) at room temperature. The reaction mixture was stirred at 100° C. for 16 hours. The reaction mixture was quenched by water (50 mL) and extracted with EA (2×50 mL). The organic layer was washed with brine (50 mL), dried over anhydrous Na₂SO₄ and concentrated under reduced pressure. The residue was purified by Pre-TLC (eluted with DCM:MeOH=15:1, v/v) to give Compound 42-2 (62 mg, 137.19 μmol, 75.2% yield). MS (ESI, m/z): 452 [M+H]⁺.

A solution of Compound 42-2 (62 mg, 137.19 μmol), INT 3 (110 mg, 214.61 μmol), cataCXium A Pd G₃ (19 mg, 26.08 μmol), Cs₂CO₃ (134 mg, 411.27 μmol) in toluene (4 mL) and water (1 mL) was stirred at 100° C. for 16 hours under nitrogen atmosphere. The reaction was diluted with EtOAc (50 mL) and washed with water (3×30 mL). The organic layer was dried over anhydrous Na₂SO₄ and concentrated under reduced pressure. The residue was purified by Pre-TLC (eluted with DCM:MeOH=15:1, v/v) to give Compound 42-3 (74 mg, 92.26 μmol, 67.3% yield). MS (ESI, m/z): 802 [M+H]⁺.

To a solution of Compound 42-3 (74 mg, 92.26 μmol) in CH₃CN (5 mL) was added HCl (4 M in 1,4-dioxane, 1 mL). The reaction mixture was stirred at room temperature for 1 hour. The resulting mixture was concentrated under reduced pressure. The residue was dissolved in EtOAc (50 mL), washed with saturated NaHCO₃ aqueous solution (3×30 mL). The organic layer was dried over anhydrous Na₂SO₄ and concentrated under reduced pressure to give Compound 42-4 (72 mg, 94.99 μmol, 102.9% yield). MS (ESI, m/z): 758 [M+H]⁺.

To a solution of Compound 42-4 (72 mg, 94.99 μmol) in DMF (3 mL) was added CsF (0.30 g, 1.97 mmol). The reaction mixture was stirred at 40° C. for 3 hours then filtered to collect the filtrate. The filtrate was concentrated under reduced pressure. The residue was purified by Prep-HPLC (YMC-Triart C18-S12 nm, phase A: 0.1% TFA in water, phase B: CH₃CN, Gradient: 15% B to 60% B in 31 min at a flow rate of 70 mL/min, 236 nm) and freeze-dried to give Compound 42 (41.7 mg, 58.26 μmol, TFA salt, 61.3% yield). MS (ESI, m/z): 602 [M+H]⁺.

### Example 43

### 5-ethynyl-6-fluoro-4-(8-fluoro-2-(((2R,7aS)-2-fluorotetrahydro-1H-pyrrolizin-7a(5H)-yl)methoxy)-4-(7-oxa-4-azaspiro[2.6]nonan-4-yl)pyrido[4,3-d]pyrimidin-7-yl)naphthalen-2-ol trifluoroacetic acid (“Compound 43”)

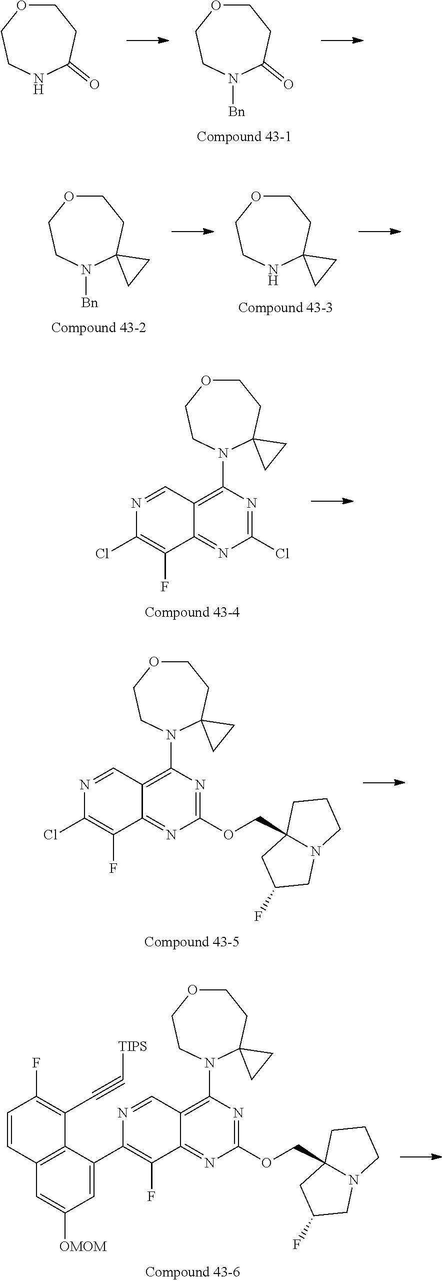

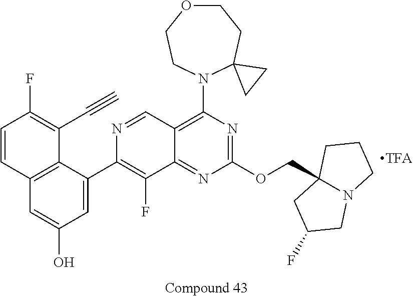 [SMILES: C#Cc1c(F)ccc2cc(O)cc(-c3ncc4c(N5CCOCCC56CC6)nc(OC[C@@]56CCCN5C[C@H](F)C6)nc4c3F)c12]

To a solution of 1,4-oxazepan-5-one (0.99 g, 8.60 mmol) and BnBr (3.77 g, 22.04 mmol) in THF (8 mL) was added sodium hydride (0.96 g, 24.00 mmol, 60% content) at 0° C. The mixture was stirred at room temperature for 1 hour. The reaction mixture was diluted with water (30 mL). The organic layer was separated and concentrated under reduced pressure. The residue was purified by silica gel chromatography (eluted with Hex:EtOAc=1:10˜ 1:2, v/v) to give Compound 43-1 (1.74 g, 8.48 mmol, 98.58% yield). MS m/z: 206 [M+H]⁺.

To a solution of Compound 43-1 (1.65 g, 8.04 mmol) in THF (20 mL) was added Ti(OiPr)₄ (4.75 g, 16.71 mmol) dropwise. Then ethylmagnesium bromide (2 M, 16 mL) was added dropwise. The reaction mixture was stirred overnight at room temperature. The reaction mixture was diluted with water (30 mL), filtered and the filtrate was extracted with EtOAc (2×30 mL). The organic layers were combined, dried over anhydrous Na₂SO₄ and concentrated under reduced pressure. The residue was purified by silica gel chromatography (eluted with DCM:MeOH=20:1, v/v) to give Compound 43-2 (527 mg, 2.43 mmol, 30.17% yield). MS m/z: 218 [M+H]⁺.

To a solution of Compound 43-2 (527 mg, 2.43 mmol) in methanol (10 mL) was added Pd/C (0.10 g, 20% content). The reaction mixture was stirred at room temperature for 10 hours under hydrogen atmosphere. The reaction mixture was filtered and the filter cake was washed with methanol. The filtrate was concentrated under reduced pressure to give Compound 43-3 (0.27 g, 2.12 mmol, 87.54% yield). MS m/z: 128 [M+H]⁺.

A solution of INT 1 (212 mg, 0.84 mmol), DIEA (238 mg, 1.84 mmol) and Compound 43-3 (0.14 g, 1.10 mmol) in DCM (10 mL) was stirred at room temperature for 3 hours. The reaction mixture was concentrated under reduced pressure. The residue was purified by Pre-TLC (eluted with Hex:EtOAc=2:1, v/v) to give Compound 43-4 (40 mg, 0.12 mmol, 13.88% yield). MS m/z: 343 [M+H]⁺.

To a solution of Compound 43-4 (40 mg, 0.12 mmol) and INT 2 (75 mg, 0.47 mmol) in DMSO (3 mL) was added KF (66 mg, 1.14 mmol). The reaction mixture was stirred at 100° C. for 3 hours under nitrogen atmosphere. The mixture was allowed to cool to room temperature, diluted with water (20 mL) and extracted with EtOAc (2×20 mL). The organic layers were combined, dried over anhydrous Na₂SO₄ and concentrated under reduced pressure. The residue was purified by Pre-TLC (eluted with DCM:MeOH=10:1, v/v) to give Compound 43-5 (77 mg, crude). MS m/z: 466 [M+H]⁺.

To a solution of Compound 43-5 (77 mg, crude), INT 3 (129 mg, 0.25 mmol) in toluene (4 mL) and water (1 mL) were added Cs₂CO₃ (203 mg, 0.62 mmol) and cataCXium A Pd G₃ (24 mg, 0.033 mmol). The reaction mixture was stirred at 100° C. overnight under nitrogen atmosphere. The reaction mixture was concentrated under reduced pressure. The residue was purified by Pre-TLC (eluted with DCM:MeOH=15:1, v/v) to give Compound 43-6 (31 mg). MS m/z: 816 [M+H]⁺.

A solution of Compound 43-6 (31 mg, 0.038 mmol) and HCl (2 mL, 4 M in dioxane) in acetonitrile (8 mL) was stirred at room temperature for 1 hour. The solution was concentrated under reduced pressure. The residue was dissolved in DMF (5 mL) and CsF (1.28 g, 8.42 mmol) was added. The reaction mixture was stirred at room temperature overnight. The mixture was diluted with water (30 mL) and extracted with EtOAc (2×30 mL). The combined organic layer was washed with brine, dried over anhydrous Na₂SO₄ and concentrated under reduced pressure. The residue was purified by Prep-HPLC (Agela Venusil Prep G C18, 30 mm×250 mm, 10 m column, A: 0.1% TFA in water, B: CH₃CN, Gradient: 15% B to 36% B in 26 min at a flow rate of 40 mL/min, 230 inm) and freeze-dried to give Compound 43 (7.8 mg, 28.14% yield, TFA salt). MS m/z: 616 [M+H]⁺.

### Example 44

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl acetyl-L-tryptophanate trifluoroacetic acid (“Compound 44”)

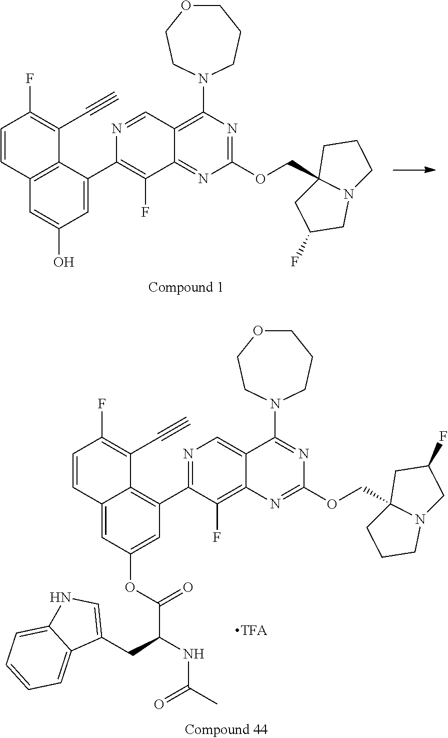 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)[C@H](Cc3c[nH]c4ccccc34)NC(C)=O)cc(-c3ncc4c(N5CCCOCC5)nc(OC[C@@]56CCCN5C[C@H](F)C6)nc4c3F)c12]

A mixture of Compound 1 (50 mg, 0.085 mmol), acetyl-L-tryptophan (37 mg, 0.15 mmol), HOBT (27 mg, 0.20 mmol), EDCI (34 mg, 0.18 mmol) and DIEA (123 mg, 0.95 mmol) in DCM (5 mL) was stirred for 2 hours at room temperature. Another batch of HOBT (37 mg) and EDCI (81 mg) were added. The mixture was stirred for 4 hours at room temperature, then diluted with water (20 mL) and extracted with DCM (2×20 mL). The combined organic layers were dried over anhydrous Na₂SO₄, filtered and concentrated under reduced pressure. The residue was purified by Prep-HPLC (Agela Venusil Prep G C18, 50 mm×250 mm, 10 m column, A: 0.05% TFA in water, B: CH₃CN, Gradient: 15% B to 45% B in 28 min to 65% B in 30 min at a flow rate of 60 mL/min, 240 nm) and freeze-dried to give Compound 44 (49.9 mg, 63.14% yield, TFA salt). MS m/z: 818 [M+H]⁺.

### Example 45

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl isobutyrate trifluoroacetic acid (“Compound 45”)

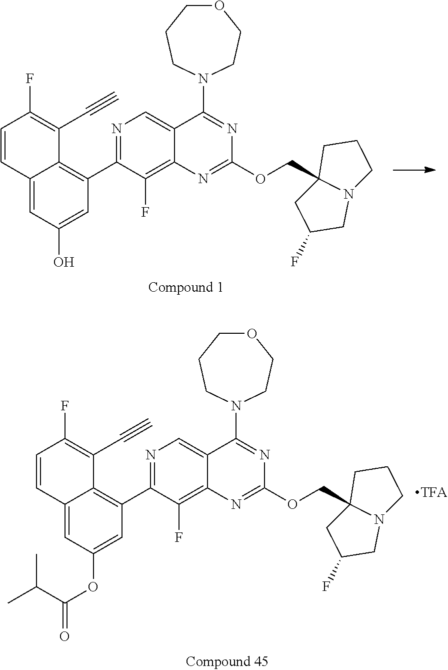 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)C(C)C)cc(-c3ncc4c(N5CCCOCC5)nc(OC[C@@]56CCCN5C[C@H](F)C6)nc4c3F)c12]

To a solution of isobutyric acid (0.51 g, 5.79 mmol) and oxalyl chloride (1.01 g, 7.96 mmol) in DCM (10 mL) was added DMF (1 drop), then the mixture was stirred at room temperature for 2 hours. The mixture was concentrated under reduced pressure at 10° C. The residue was dissolved with DCM (1 mL) then 3 drops of the mixture was added to a solution of Compound 1 (44 mg, 0.075 mmol) and triethylamine (0.5 mL) in DCM (10 mL). The reaction mixture was stirred at room temperature for 0.5 h. After completion, the reaction mixture was concentrated under reduced pressure. The residue was purified by Pre-HPLC (C18 column, A: 0.05% TFA in water, B: CH₃CN, Gradient: 25% B to 70% B in 60 min at a flow rate of 70 mL/min, 240 nm) and freeze-dried to give Compound 45 (47.1 mg, TFA salt, 0.061 mmol). MS: m/z 660 [M+H]⁺.

### Example 46

### (R)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (“Compound 46A” or “Compound 46B”)

(S)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (“Compound 46B” or “Compound 46A”)

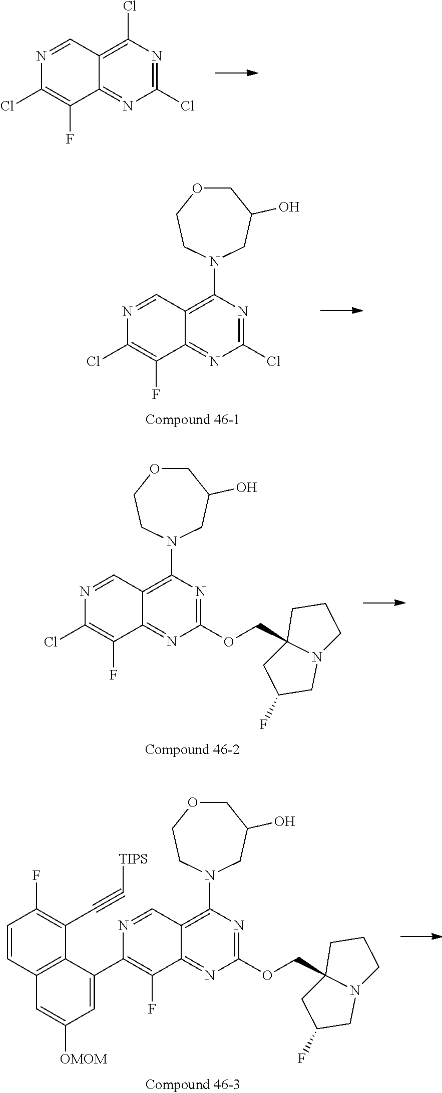

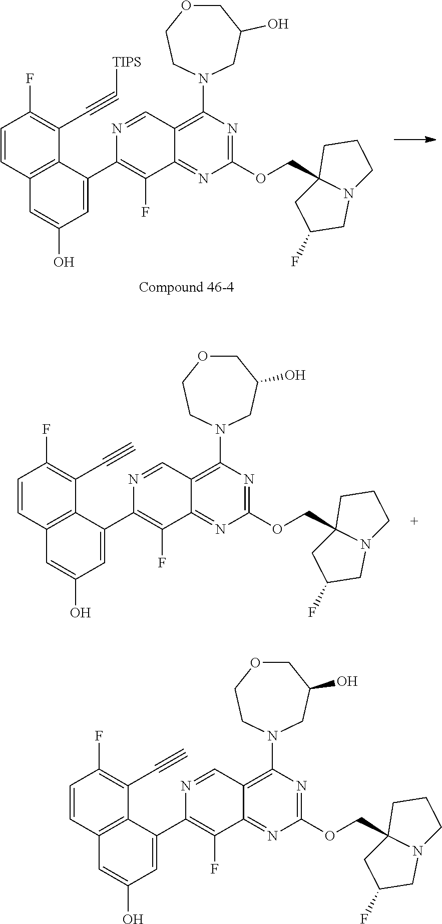

To a solution of tert-butyl 6-hydroxy-1,4-oxazepane-4-carboxylate (142 mg, 0.65 mmol) in DCM (10 mL) was added HCl (4M in 1,4-dioxane, 2 mL). The reaction mixture was stirred at room temperature for 1 hour. After completion, the reaction mixture was concentrated under reduced pressure. The residue was diluted with DCM (10 mL) and 2,4,7-trichloro-8-fluoropyrido[4,3-d]pyrimidine (162 mg, 0.64 mmol) and DIEA (0.5 mL) were added. The reaction mixture was stirred at room temperature. Upon completion, the solution was diluted with water (30 mL) and extracted with DCM (40 mL). The organic layer was washed with 5% citric acid (30 mL), brine (30 mL), dried over anhydrous Na₂SO₄ and concentrated under reduced pressure to give Compound 46-1 (239 mg, 0.72 mmol). MS: m/z 333[M+H]⁺.

A solution of Compound 46-1 (239 mg, 0.72 mmol), INT 2 (159 mg, 1.00 mmol) and KF (165 mg, 2.84 mmol) in DMSO (8 mL) was stirred at 85° C. for 16 hours under nitrogen atmosphere. The mixture was diluted with EtOAc (50 mL) and water (40 mL) and the organic layer was separated. The organic layer was washed with brine (40 mL), dried over anhydrous Na₂SO₄, filtered and concentrated under reduced pressure. The residue was purified by Pre-TLC (eluted with DCM:MeOH=15:1, v/v) to give Compound 46-2 (122 mg, 0.27 mmol). MS: m/z 456 [M+H]⁺.

To a solution of Compound 46-2 (122 mg, 0.27 mmol), INT 3 (177 mg, 0.35 mmol) and Cs₂CO₃ (272 mg, 0.83 mmol) in toluene (8 mL) and water (2 mL) was added cataCXium A Pd G₃ (24 mg, 32.95 μmol). The mixture was purged with nitrogen followed by stirring at 100° C. for 16 hours. The mixture was allowed to cool to room temperature, then diluted with water (30 mL) and extracted with EtOAc (40 mL). The organic layer was washed with brine, dried over anhydrous Na₂SO₄ and concentrated under reduced pressure. The residue was purified by Pre-TLC (eluted with DCM:MeOH=15:1, v/v) to give Compound 46-3 (74 mg, 0.092 mmol). MS: m/z 806 [M+H]⁺.

To a solution of Compound 46-3 (74 mg, 0.092 mmol) in CH₃CN (5 mL) was added HCl/1,4-dioxnae (4 M, 2 mL). The reaction mixture was stirred at room temperature for 1 hour. After completion, the reaction mixture was concentrated under reduced pressure. The residue was diluted with EtOAc (40 mL) and water (30 mL) then the mixture was adjusted to pH 8-9 with saturated NaHCO₃ aqueous solution. The organic layer was separated and concentrated under reduced pressure to give crude Compound 46-4 (72 mg, crude). MS: m/z 762 [M+H]⁺.

To a mixture of Compound 46-4 (72 mg, crude) in DMF (5 mL) was added CsF (242 mg, 1.59 mmol). The mixture was stirred at room temperature for 16 hours. After completion, the mixture was diluted with EtOAc (40 mL) and water (30 mL). The mixture was adjusted to pH 8-9 with saturated NaHCO₃ aqueous solution. The organic layer was washed with brine then dried over anhydrous Na₂SO₄ and concentrated under reduced pressure. The residue was purified by Prep-HPLC (C18 column, A: 0.1% TFA in water, B: CH₃CN, Gradient: 15% B to 35% B in 60 min at a flow rate of 70 mL/min, 240 nm). Then the two isomers were separated by Prep-HPLC-Gilson with the following conditions: Column, CHIRAL ART Cellulose-SA column (2 cm×25 cm, 5 um), mobile phase, (Hex:DCM=3:1) (0.1% isopropylamine)/EtOH (93:7); Flowing rate: 20 mL/min. This results in Compound 46A (6.5 mg, the first eluting isomer, Retention Time 10.059 min) and Compound 46B (5.6 mg, the second eluting isomer, Retention Time 11.455 min). MS: m/z 606[M+H]⁺.

### Example 47

### 5-ethynyl-6-fluoro-4-(8-fluoro-2-(((2R,7aS)-2-fluorotetrahydro-1H-pyrrolizin-7a(5H)-yl)methoxy)-5-(methylthio)-4-(1,4-oxazepan-4-yl)pyrido[4,3-d]pyrimidin-7-yl)naphthalen-2-ol trifluoroacetic acid (“Compound 47”)

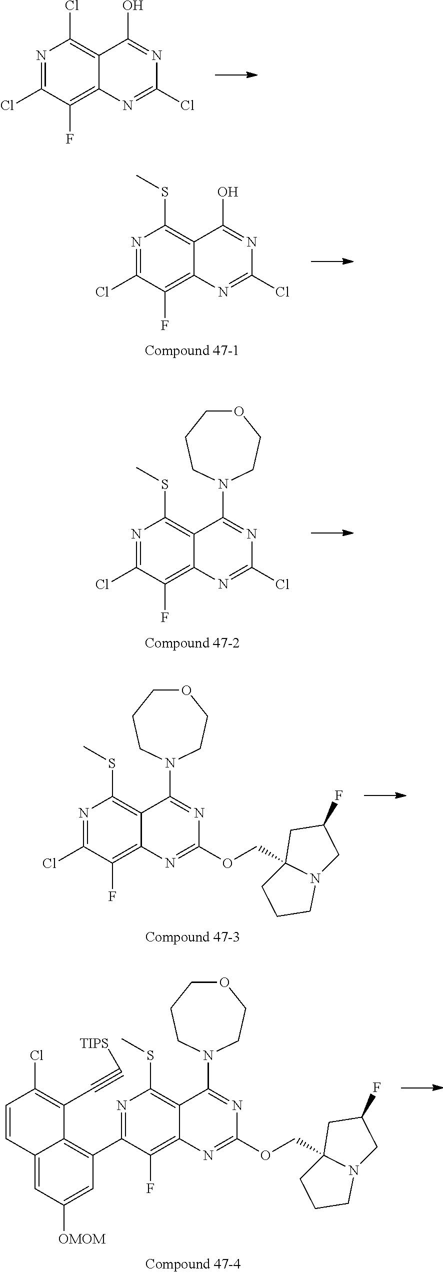

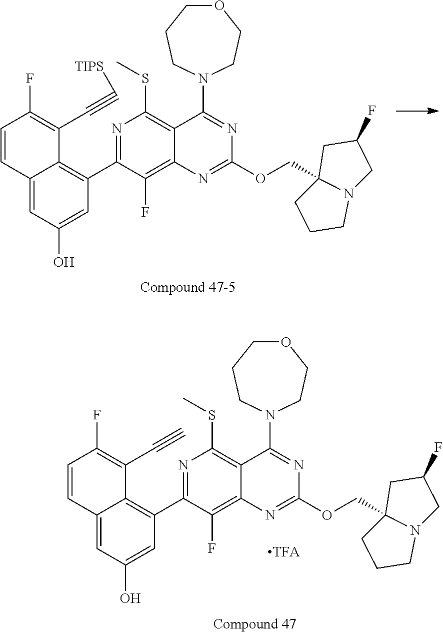

To a solution of 2,5,7-trichloro-8-fluoropyrido[4,3-d]pyrimidin-4-ol (509 mg, 1.89 mmol) in DMAc (10 mL) was added sodium thiomethoxide (263 mg, 3.75 mmol). The reaction mixture was stirred at 100° C. for 2 hours. After cooling to room temperature, the mixture was diluted with EA (50 mL) and washed with HCl aqueous solution (1 N, 3×30 mL). The organic layer was dried over anhydrous Na₂SO₄ and concentrated under vacuum to give Compound 47-1 (0.5 g, 1.78 mmol, 94.1% yield). MS (ESI, m/z): 278 [M−H]—.

To a solution of Compound 47-1 (309 mg, 1.10 mmol) in CH₃CN (6 mL) were added POCl₃ (563 mg, 3.67 mmol) and N,N-diisopropylethylamine (432 mg, 3.34 mmol). The reaction mixture was stirred at 80° C. for 2 hours. After cooling to 0° C., N,N-diisopropylethylamine was added and the pH was adjusted to8, then 1,4-oxazepane (218 mg, 2.15 mmol) was added and the mixture was stirred at room temperature for 2 hours. The mixture was concentrated under vacuum and the residue was purified by RP-flash, eluting with 5-70% CH₃CN in H₂O (0.1% TFA) to give Compound 47-2 (169 mg, 465.26 μmol, 42.1% yield). MS (ESI, m/z): 363 [M+H]⁺.

To a solution of Compound 47-2 (169 mg, 465.26 μmol) and INT 2 (97 mg, 609.29 μmol) in THF (6 mL) was added sodium tert-butoxide (104 mg, 1123.79 μmol) at room temperature and then the mixture was stirred at room temperature for 2 hours. The mixture was quenched with saturated NH₄Cl aqueous solution (30 mL) and extracted with EA (50 mL). The organic layer was dried over anhydrous Na₂SO₄ and concentrated under vacuum. The residue was purified by Pre-TLC (DCM:MecOH=15:1, v/v) to give Compound 47-3 (156 mg, 321.00 μmol, 69.0% yield). MS (ESI, m/z): 486 [M+H]⁺.

A solution of Compound 47-3 (156 mg, 321.00 μmol), INT 3 (340 mg, 663.36 μmol), cataCXium A Pd G₃ (45 mg, 61.79 μmol), Cs₂CO₃ (316 mg, 969.86 μmol) in toluene (6 mL) and water (1.5 mL) was stirred at 100° C. for 16 hours under nitrogen atmosphere. The reaction was diluted with EA (50 mL) and washed with water (3×30 mL). The organic layer was dried over anhydrous Na₂SO₄ and concentrated under vacuum. The residue was purified by Pre-TLC (DCM:MeOH=15:1, v/v) to give Compound 47-4 (206 mg, 246.38 μmol, 76.7% yield). MS (ESI, m/z): 836 [M+H]⁺.

To a solution of Compound 47-4 (49 mg, 58.60 μmol) in CH₃CN (5 mL) was added HCl (4 M in 1,4-dioxane, 1 mL). The reaction mixture was stirred at room temperature for 1 hour. The resulting mixture was concentrated under reduced pressure. The residue was dissolved in EA (50 mL) and washed with saturated NaHCO₃ aqueous solution (3×30 mL). The organic layer was dried over anhydrous Na₂SO₄ and concentrated under reduced pressure to give Compound 47-5 (51 mg, 64.39 μmol, 109.8% yield). MS (ESI, m/z): 792 [M+H]⁺.

To a solution of Compound 47-5 (51 mg, 64.39 μmol) in DMF (3 mL) was added CsF (0.26 g, 1.71 mmol). The reaction mixture was stirred at 40° C. for 16 h then filtered to collect the filtrate. The filtrate was concentrated under reduced pressure. The residue was purified by Prep-HPLC (Daisogel-C18, phase A: 0.1% TFA in water, phase B: CH₃CN, Gradient: 15% B to 45% B in 45 min at a flow rate of 60 mL/min, 240 nm) and freeze-dried to give Compound 47 (20.9 mg, 27.87 μmol, TFA salt, 43.2% yield). MS (ESI, m/z): 636 [M+H]⁺.

### Example 48

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl cinnamate trifluoroacetic acid (“Compound 48”)

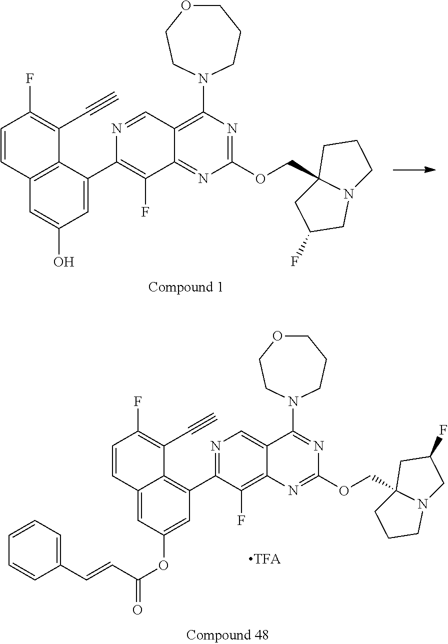 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)/C=C/c3ccccc3)cc(-c3ncc4c(N5CCCOCC5)nc(OC[C@@]56CCCN5C[C@H](F)C6)nc4c3F)c12]

A mixture of Compound 1 (50 mg, 0.085 mmol), cinnamic acid (49 mg, 0.33 mmol), HOBT (32 mg, 0.24 mmol), EDCI (36 mg, 0.19 mmol) and DIEA (140 mg, 1.08 mmol) in DMF (5 mL) was stirred for 3 hours at room temperature. The mixture was diluted with water (30 mL) and extracted with EtOAc (30 mL). The organic layer was dried over anhydrous Na₂SO₄, filtered and concentrated under reduced pressure. The residue was purified by Prep-HPLC (Daisogel C18 column, 50 mm×250 mm, 10 m; A: 0.1% TFA in water, B: CH₃CN, Gradient: 15% B to 60% B in 50 min at a flow rate of 60 mL/min, 230 nm) and freeze-dried to give Compound 48 (51.8 mg, 73.26% yield, TFA salt). MS m/z: 720 [M+H]⁺.

### Example 49

### 5-ethynyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol (“Compound 49A” or “Compound 49B”)

### 5-ethynyl-6-fluoro-4-(8-fluoro-2-(((2R,7aS)-2-fluorotetrahydro-1H-pyrrolizin-7a(5H)-yl)methoxy)-4-((R)-6-methoxy-1,4-oxazepan-4-yl)pyrido[4,3-d]pyrimidin-7-yl)naphthalen-2-ol (“Compound 49B” or “Compound 49A”)

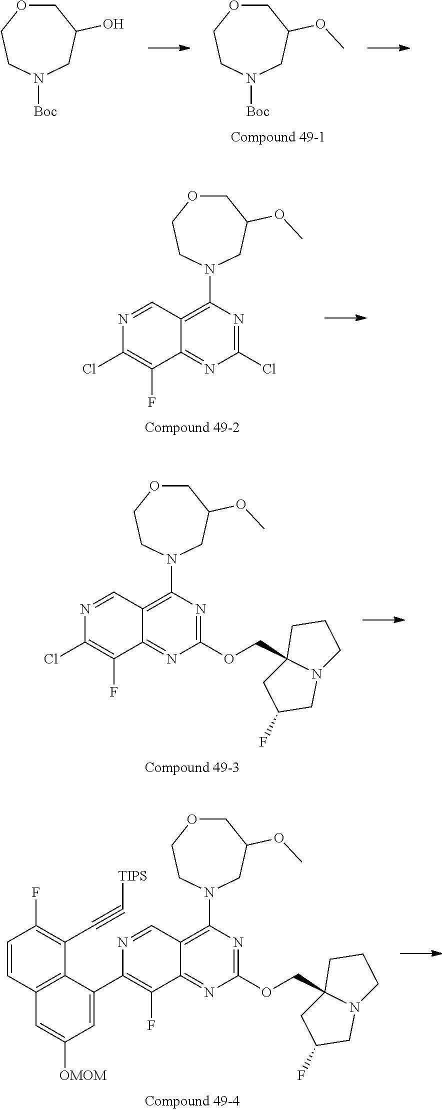

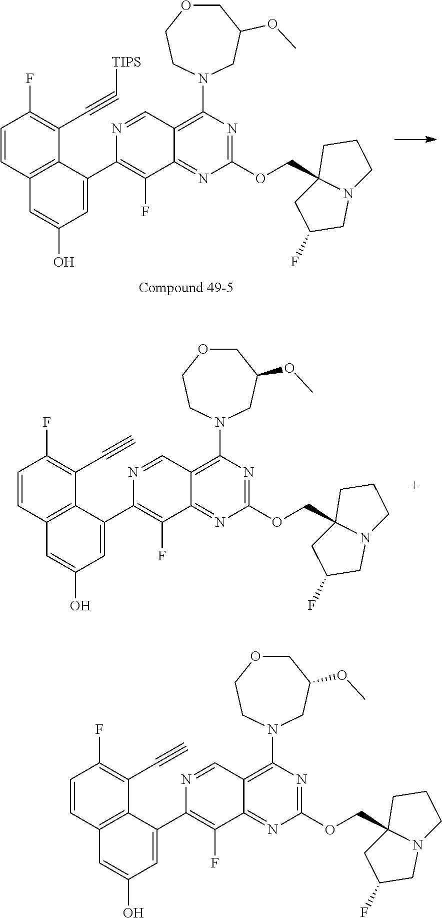

To solution of tert-butyl 6-hydroxy-1,4-oxazepane-4-carboxylate (112 mg, 0.44 mmol) in THF (10 mL) was added NaH (98 mg, 2.45 mmol) at 0° C. and the mixture was stirred for 1 hour. The mixture was allowed to cool to room temperature and CH₃I (228 mg, 1.61 mmol) was added. Upon completion, the mixture was diluted with water (30 mL) and extracted with EtOAc (40 mL). The organic layer was washed with brine (30 mL), dried over anhydrous Na₂SO₄ and concentrated under reduced pressure to give Compound 49-1 (208 mg, 0.90 mmol). MS: m/z 232 [M+H]⁺.

To solution of Compound 49-1 (208 mg, 0.90 mmol) in DCM (10 mL) was added HCl (4 M in 1, 4-dioxane, 2 mL). The reaction mixture was stirred at room temperature for 1 hour. After completion, the reaction mixture was concentrated under reduced pressure. The residue was diluted with DCM (10 mL), and INT 1 (209 mg, 0.83 mmol) and DIEA (0.5 mL) were added. Upon completion, the solution was diluted with water (30 mL) and extracted with EtOAc (40 mL). The organic layer was washed with brine (30 mL), dried over anhydrous Na₂SO₄ and concentrated under reduced pressure to give Compound 49-2 (323 mg, 0.93 mmol). MS: m/z 347 [M+H]⁺.

To a solution of Compound 49-2 (323 mg, 0.93 mmol), INT 2 (205 mg, 1.29 mmol) and KF (225 mg, 3.87 mmol) in DMSO (8 mL) was stirred at 85° C. for 16 hours under nitrogen atmosphere. The mixture was diluted with EtOAc (50 mL) and water (40 mL), and the organic layer was separated. The organic layer was washed with brine (40 mL), dried over anhydrous Na₂SO₄, filtered and concentrated under reduced pressure. The residue was purified by Pre-TLC (eluted with DCM:MeOH=20:1, v/v) to give Compound 49-3 (228 mg, 0.49 mmol). MS: m/z 470 [M+H]⁺.

To a solution of Compound 49-3 (104 mg, 0.22 mmol), INT 3 (157 mg, 0.31 mmol) and Cs₂CO₃ (222 mg, 0.68 mmol) in toluene (8 mL) and water (2 mL) was added cataCXium A Pd G₃ (21 mg, 28.84 μmol). The mixture was purged with nitrogen followed by stirring at 100° C. for 16 hours. The mixture was allowed to cool to room temperature, then diluted with water (30 mL) and extracted with EtOAc (40 mL). The organic layer was washed with brine, dried over anhydrous Na₂SO₄ and concentrated under reduced pressure. The residue was purified by Pre-TLC (eluted with DCM:MeOH=20:1, v/v) to give Compound 49-4 (94 mg, 0.11 mmol). MS: m/z 820 [M+H]⁺.

To a solution of Compound 49-4 (94 mg, 0.11 mmol) in CH₃CN (5 mL) was added HCl (4 M in 1,4-dioxane, 2 mL). The reaction mixture was stirred at room temperature for 1 hour. After completion, the reaction mixture was concentrated under reduced pressure. The residue was diluted with EtOAc (40 mL) and water (30 mL). The mixture was adjusted to pH 8-9 with saturated NaHCO₃ aqueous solution. The organic layer was separated and concentrated under reduced pressure to give Compound 49-5 (108 mg, crude). MS: m/z 776 [M+H]⁺.

To a mixture of Compound 49-5 (108 mg, crude) in DMF (5 mL) was added CsF (247 mg, 1.63 mmol). The mixture was stirred at 40° C. for 18 hours. After completion, the mixture was diluted with EtOAc (40 mL) and water (30 mL). The mixture was adjusted to pH 8-9 with saturated NaHCO₃ aqueous solution. The organic layer was washed with brine, dried over anhydrous Na₂SO₄ and concentrated under reduced pressure. The residue was purified by Prep-HPLC (C18 column, A: 0.1% FA in water, B: CH₃CN, Gradient: 10% B to 40% B in 60 min at a flow rate of 70 mL/min, 240 nm). Then the two isomers were separated by Prep-HPLC-Gilson with the following conditions: Column, CHIRAL ART Cellulose-SA column (2 cm×25 cm, 5 um); mobile phase, (Hex:DCM=3:1) (0.1% isopropylamine)/EtOH (90:10); Flowing rate: 20 mL/min to give Compound 49A (12.1 mg, the first eluting isomer, Retention Time 6.132 min) and Compound 49B (15.9 mg, the second eluting isomer, Retention Time 6.693 min). MS: m/z 620[M+H]⁺.

### Example 50

### 5-ethynyl-6-fluoro-4-(8-fluoro-2-(((2R,7aS)-2-fluorotetrahydro-1H-pyrrolizin-7a(5H)-yl)methoxy)-4-(8-oxa-4-azaspiro[2.6]nonan-4-yl)pyrido[4,3-d]pyrimidin-7-yl)naphthalen-2-ol trifluoroacetic acid (“Compound 50”)

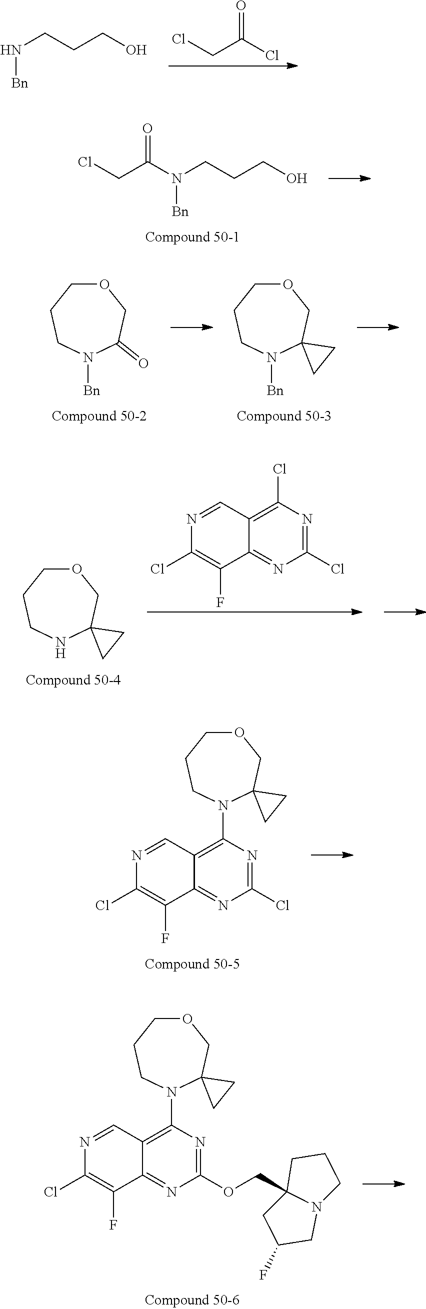

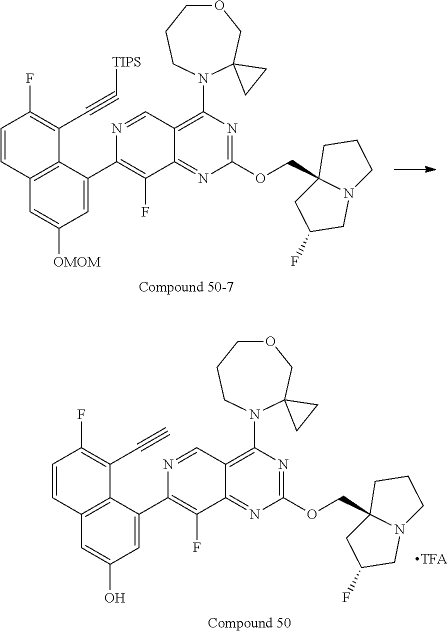

To a solution of N-benzyl-3-aminopropan-1-ol (1.03 g, 6.23 mmol) and Na₂CO₃ (1.28 g, 12.08 mmol) in DCM (5 mL) was added monochloroacetyl chloride (0.69 g, 6.11 mmol). The reaction mixture was stirred at room temperature for 1.5 hours under nitrogen atmosphere. The mixture was concentrated under reduced pressure. The residue was purified by silica gel chromatography (eluted with DCM:MeOH=100:1 to 50:1, v/v) to give Compound 50-1 (776 mg, 3.21 mmol, 54.50% yield). MS m/z: 242 [M+H]⁺.

To a solution of Compound 50-1 (723 mg, 2.99 mmol) in THF (8 mL) was added NaH (365 mg, 9.13 mmol, 60% wt) portion-wise. The reaction mixture was stirred at room temperature for 3 hours under nitrogen atmosphere. The mixture was concentrated under reduced pressure. The residue was purified by silica gel chromatography (eluted with DCM:MeOH=100:1 to 50:1, v/v) to give Compound 50-2 (465 mg, 2.27 mmol, 75.74% yield). MS m/z: 206 [M+H]⁺.

To a solution of Compound 50-2 (465 mg, 2.27 mmol) and titanium tetraisopropanolate (1.34 g, 4.71 mmol) in THF (10 mL) was added ethylmagnesium bromide (4.5 mL, 2M) dropwise. The reaction mixture was stirred at room temperature overnight under nitrogen atmosphere. The mixture was quenched with water (2 mL) then filtered. The filtrate was concentrated under reduced pressure. The residue was purified by silica gel chromatography (DCM:MeOH=100:1, v/v) to give Compound 50-3 (192 mg, 0.884 mmol, 39.00% yield). MS m/z: 218 [M+H]⁺.

To a solution of Compound 50-3 (192 mg, 0.884 mmol) in MeOH (8 mL) was added Pd(OH)₂/C (754 mg, 1.07 mmol, 20% wt). The reaction mixture was stirred at room temperature for 3 h under hydrogen atmosphere. The mixture was filtered and the filtrate was concentrated under reduced pressure to give Compound 50-4 (72 mg, 0.566 mmol, 64.07% yield). MS m/z: 128 [M+H]⁺.

To a solution of Compound 50-4 (72 mg, 0.566 mmol) and DIEA (0.3 mL) in DCM (4 mL) was added INT 1 (143 mg, 0.566 mmol) at 0° C. The mixture was stirred at room temperature for 1 hour. The reaction mixture was diluted with DCM (20 mL). The collected organic layer was washed with water (2×20 mL), dried over anhydrous Na₂SO₄ and concentrated under reduced pressure. The residue was purified by Pre-TLC (eluted with DCM:MeOH=40:1, v/v) to give Compound 50-5 (102 mg, 0.297 mmol, 52.50% yield). MS m/z: 343/345 [M+H]⁺.

To a solution of Compound 50-5 (102 mg, 0.297 mmol) and INT 2 (83 mg, 0.521 mmol) in DMSO (4 mL) was added KF (56 mg, 0.964 mmol). The reaction mixture was stirred at 88° C. overnight under nitrogen atmosphere. The mixture was allowed to cool to room temperature and extracted with EtOAc (30 mL). The organic layer was washed with brine (2×30 mL), dried over anhydrous Na₂SO₄ and concentrated under reduced pressure. The residue was purified by Pre-TLC (eluted with DCM:MeOH=15:1, v/v) to give Compound 50-6 (91 mg, 0.195 mmol, 65.71% yield). MS m/z: 466 [M+H]⁺.

To a solution of Compound 50-6 (91 mg, 0.195 mmol) and INT 3 (141 mg, 0.275 mmol) in toluene (5 mL) and water (1 mL) were added Cs₂CO₃ (193 mg, 0.592 mmol) and cataCXium A Pd G₃ (30 mg, 0.041 mmol). The reaction mixture was stirred at 100° C. overnight under nitrogen atmosphere. The reaction mixture was filtered and the filtrate was concentrated under reduced pressure. The residue was purified by Pre-TLC (eluted with DCM:MeOH=20:1, v/v) to give Compound 50-7 (103 mg, 0.126 mmol, 64.62% yield). MS m/z: 816 [M+H]⁺.

A solution of Compound 50-7 (103 mg, 0.126 mmol) and HCl (0.8 mL, 4 M in dioxane) in MeCN (3 mL) was stirred at room temperature for 1 hour. The mixture was concentrated under reduced pressure. The residue was diluted with saturated NaHCO₃ aqueous solution (20 mL) and extracted with EtOAc (2×20 mL). The collected organic layers were combined, dried over anhydrous Na₂SO₄ and concentrated under reduced pressure. To the residue in DMF (4 mL) was added CsF (300 mg, 1.97 mmol). The reaction mixture was stirred at 45° C. for 2 hours under nitrogen atmosphere. The reaction mixture was purified by Prep-HPLC (C18 column, A: 0.05% TFA in water, B: CH₃CN, Gradient: 15% B to 50% B in 60 min at a flow rate of 70 mL/min, 240 nm) and freeze-dried to give Compound 50 (37.4 mg, 0.051 mmol, TFA salt). MS m/z: 616 [M+H]⁺.

### Example 51

### 4-(5-cyclopropoxy-8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)-5-ethynyl-6-fluoronaphthalen-2-ol trifluoroacetic acid (“Compound 51”)

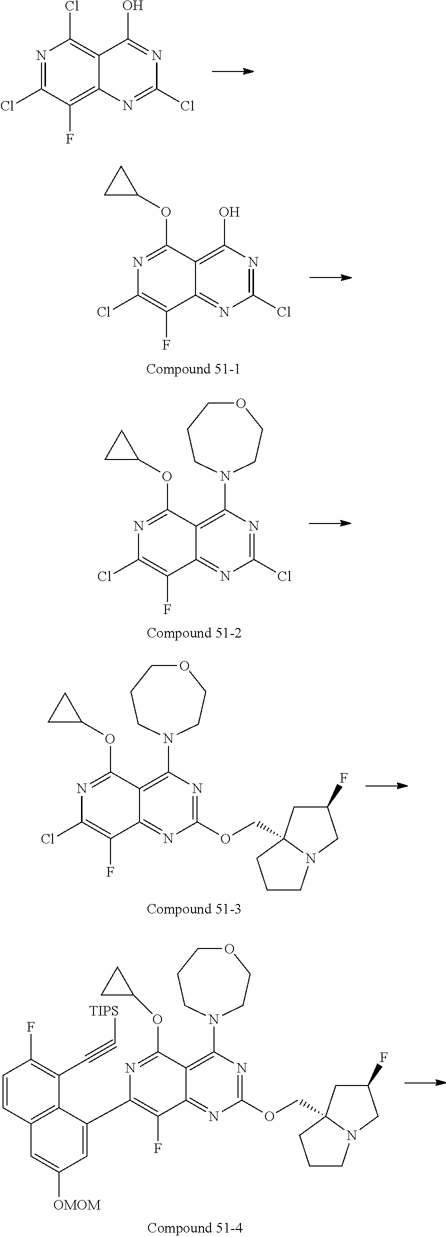

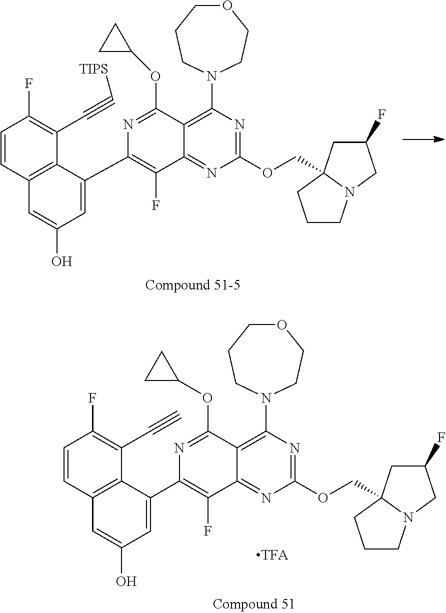

To a solution of cyclopropanol (98 mg, 1.68 mmol) in THF (10 mL) was added NaH (84 mg, 2.10 mmol) at 0° C. The mixture was stirred at room temperature for 30 min. After the mixture was cooled to 0° C., 2,5,7-trichloro-8-fluoropyrido[4,3-d]pyrimidin-4-ol (307 mg, 1.14 mmol) was added and the mixture was stirred for 3 hours. The mixture was quenched with saturated NH₄Cl aqueous solution (30 mL) and extracted with EtOAc (50 mL). The organic layer was dried over anhydrous Na₂SO₄ and concentrated under reduced pressure to give crude Compound 51-1 which was used in next step directly without further purification.

To a solution of Compound 51-1 (crude, 1.14 mmol) in toluene (10 mL) was added POCl₃ (0.5 mL) and N,N-diisopropylethylamine (0.2 mL). The reaction mixture was stirred at 100° C. for 2 hours. Then the mixture was concentrated under reduced pressure and the crude was dissolved in DCM (10 mL). N,N-diisopropylethylamine (0.2 mL) and 1,4-oxazepane (213 mg, 2.10 mmol) were added. The mixture was stirred at room temperature for 2 hours. The mixture was quenched with saturated NH₄Cl aqueous solution (30 mL) then extracted with EtOAc (50 mL). The organic layer was dried over anhydrous Na₂SO₄ and concentrated under reduced pressure. The residue was purified by Pre-TLC (Hex:EtOAc=2:1, v/v) to give Compound 51-2 (329 mg, 881.54 μmol, 77.0% yield). MS (ESI, m/z): 373 [M+H]f.

To a solution of Compound 51-2 (329 mg, 881.54 μmol) and INT 2 (284 mg, 1.78 mmol) in DMSO (10 mL) was added KF (153 mg, 2.63 mmol) at room temperature. The reaction mixture was stirred at 90° C. for 16 hours. The reaction mixture was quenched by water (50 mL) and extracted with EtOAc (2×50 mL). The organic layer was washed with brine (50 mL), dried over anhydrous Na₂SO₄ and concentrated under reduced pressure. The residue was purified by Pre-TLC (eluted with DCM:MeOH=15:1, v/v) to give Compound 51-3 (335 mg, 675.47 μmol, 76.6% yield). MS (ESI, m/z): 496 [M+H]⁺.

A solution of Compound 51-3 (97 mg, 195.58 μmol), INT 3 (153 mg, 298.51 μmol), cataCXium A Pd G₃ (29 mg, 39.82 μmol), Cs₂CO₃ (130 mg, 398.99 μmol) in toluene (6 mL) and water (1.5 mL) was stirred at 100° C. for 16 hours under nitrogen atmosphere. The reaction mixture was diluted with EtOAc (50 mL) and washed with water (3×30 mL). The organic layer was dried over anhydrous Na₂SO₄ and concentrated under reduced pressure. The residue was purified by Pre-TLC (eluted with DCM:MeOH=15:1, v/v) to give Compound 51-4 (107 mg, 126.46 μmol, 64.6% yield). MS (ESI, m/z): 846 [M+H]⁺.

To a solution of Compound 51-4 (107 mg, 126.46 μmol) in CH₃CN (5 mL) was added HCl (4 M in 1,4-dioxane, 1 mL). The reaction mixture was stirred at room temperature for 1 hour. The resulting mixture was concentrated under reduced pressure. The residue was dissolved in EtOAc (50 mL) and washed with saturated NaHCO₃ aqueous solution (3×30 mL). The organic layer was dried over anhydrous Na₂SO₄ and concentrated under reduced pressure to give Compound 51-5 (153 mg, 190.77 μmol, 150.8% yield). MS (ESI, m/z): 802 [M+H]⁺.

To a solution of Compound 51-5 (153 mg, 190.77 μmol) in DMF (3 mL) was added CsF (0.32 g, 2.10 mmol). The reaction mixture was stirred at 40° C. for 5 hours then filtered to collect the filtrate. The filtrate was concentrated under reduced pressure. The residue was purified by Prep-HPLC (Daisogel-C18, phase A: 0.1% TFA in water, phase B: CH₃CN, Gradient: 15% B to 45% B in 40 min at a flow rate of 60 mL/min, 240 nm) and freeze-dried to give Compound 51 (50.9 mg, 67.00 μmol, TFA salt, 35.1% yield). MS (ESI, m/z): 646 [M+H]⁺.

### Example 52

### 5-ethynyl-6-fluoro-4-(8-fluoro-2-(((2R,7aS)-2-fluorotetrahydro-1H-pyrrolizin-7a(5H)-yl)methoxy)-5-isopropoxy-4-(1,4-oxazepan-4-yl)pyrido[4,3-d]pyrimidin-7-yl)naphthalen-2-ol trifluoroacetic acid (“Compound 52”)

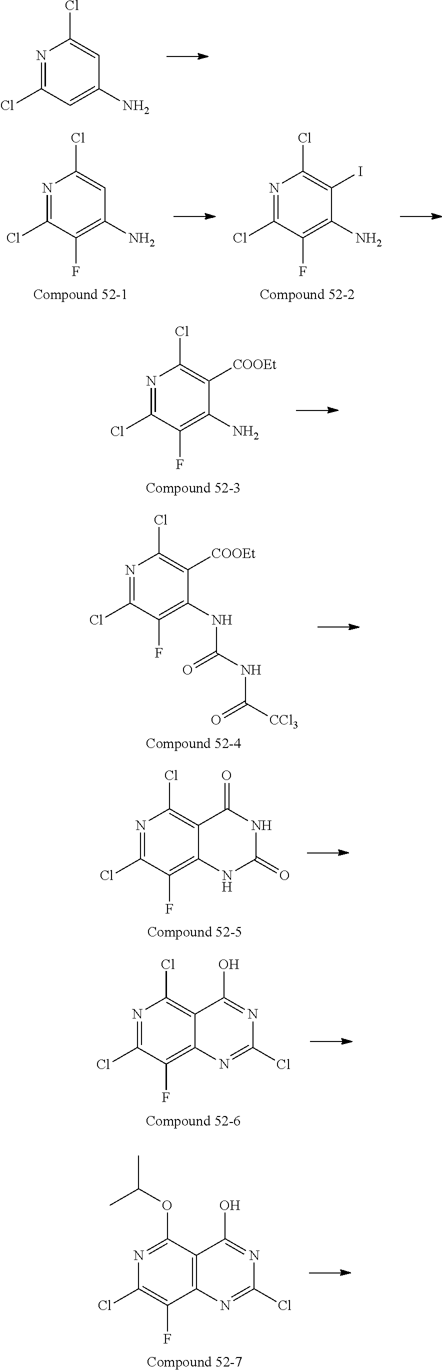

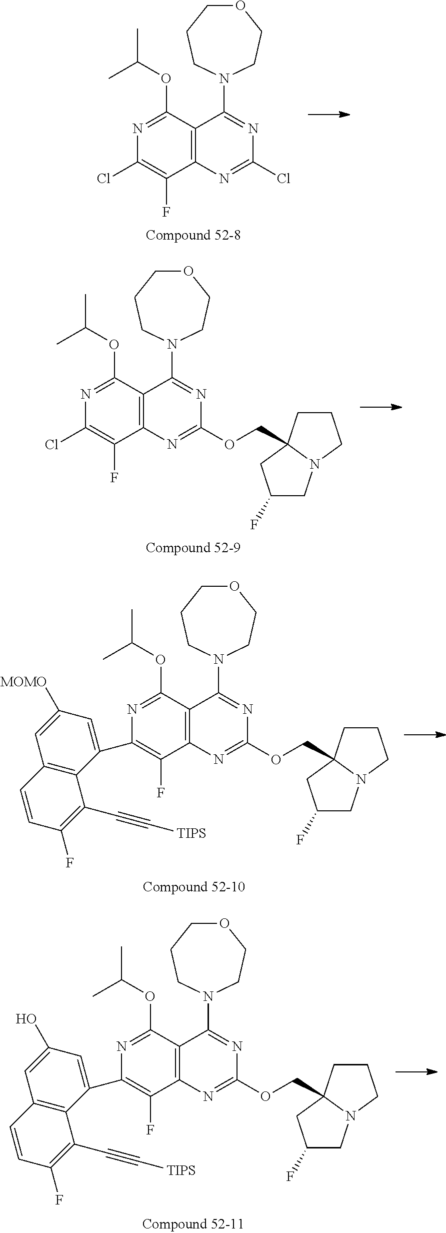

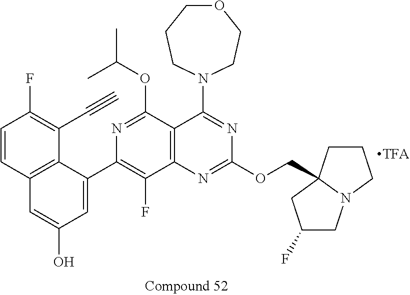 [SMILES: C#Cc1c(F)ccc2cc(O)cc(-c3nc(OC(C)C)c4c(N5CCCOCC5)nc(OC[C@@]56CCCN5C[C@H](F)C6)nc4c3F)c12]

A mixture of 2,6-dichloropyridin-4-amine (35.7 g, 219.0 mmol), 1-(chloromethyl)-4-fluoro-1,4-diazoniabicyclo[2.2.2]octane ditetrafluoroborate (93.1 g, 262.8 mmol) in DMF (357 mL) and CH₃CN (357 mL) was stirred at 80° C. for 6 hours. The reaction mixture was quenched by water (400 mL) and extracted with DCM (400 mL×3). The organic layers were combined, dried over anhydrous Na₂SO₄, filtered and concentrated under reduced pressure. The residue was purified by silica gel column (eluting with petroleum ether:EtOAc=30:1, v/v) to give Compound 52-1 (12.6 g, purity: about 50%). MS (ESI, m/z): 181 [M+H]⁺.

A mixture of Compound 52-1 (2.0 g, 11.05 mmol), NIS (2.98 g, 13.26 mmol) and p-toluenesulfonic acid monohydrate (105 mg, 0.55 mmol) in CH₃CN (8.4 mL) was stirred at 70° C. for 4 hours under nitrogen atmosphere. The reaction mixture was quenched by water (20 mL) and extracted with EtOAc (20 mL×3). The organic layers were combined, dried over anhydrous Na₂SO₄, filtered and concentrated under reduced pressure. The residue was purified by silica gel column (eluting with petroleum ether:EtOAc=50: 1-20:1, v/v) to give Compound 52-2 (3.6 g). MS (ESI, m/z): 307 [M+H]⁺.

A mixture of Compound 52-2 (1.0 g, 3.26 mmol), Pd(PPh₃)₂Cl₂ (229 mg, 0.33 mmol) and Et₃N (1.19 g, 11.77 mmol) in EtOH (17.0 mL) was stirred at 80° C. for 20 hours under carbon monoxide atmosphere (1.5 MPa) in a sealed tube. The reaction mixture was filtered and the filtrate was concentrated under reduced pressure. The residue was purified by silica gel column to give Compound 52-3 (1.2 g). MS (ESI, m/z): 253 [M+H]⁺.

A mixture of Compound 52-3 (800 mg, 3.16 mmol), trichloroacetyl isocyanate (714 mg, 3.79 mmol) in THF (8 mL) was stirred at room temperature for 1 h. The reaction mixture was concentrated under reduced pressure. The residue was triturated with MTBE to give Compound 52-4 (880 mg). MS (ESI, m/z): 442 [M+H]A mixture of Compound 52-4 (780 mg, 1.77 mmol), NH₃/MeOH (1.26 mL, 7M, 8.85 mmol) and MeOH (7.8 mL) was stirred at room temperature for 1 h. The reaction mixture was concentrated under reduced pressure. The residue was triturated with MTBE to give Compound 52-5 (550 mg). MS (ESI, m/z): 250 [M+H]+.

A mixture of Compound 52-5 (375 mg, 1.50 mmol), DIPEA (595 mg, 4.60 mmol) and POCl₃ (15 mL) was stirred at 105° C. for 17 hours. The reaction mixture was concentrated under reduced pressure. The residue was diluted with 1,4-dioxane (5 mL) and the resulting solution was added dropwise to aq. K₂CO₃ (20%, 30 mL). The mixture was stirred for 2 hours at RT and the pH of the mixture was adjusted to 2˜3. Then, the mixture was filtered and the filter cake was collected and dried to give Compound 52-6 (344 mg). MS (ESI, m/z): 268 [M+H]⁺.

To a solution of isopropyl alcohol (28 mg, 0.47 mmol) in dry THF (5 mL) was added sodium hydride (43 mg, 1.07 mmol) under nitrogen atmosphere at 0° C. The resulting mixture was stirred at room temperature for 30 minutes. A solution of 2,5,7-trichloro-8-fluoropyrido[4,3-d]pyrimidin-4-ol (148 mg, 0.55 mmol) in dry THF (1 mL) was added and stirred at 40° C. for 24 hours. The mixture was adjusted to pH 5-6 with 5% citric acid and extracted with EtOAc twice. The combined organic phase was washed with brine, dried over anhydrous Na₂SO₄, filtered and concentrated under reduced pressure. The residue was purified by Pre-TLC (eluted with DCM:MeOH=10:1, v/v) to give Compound 52-7 (99.8 mg, 0.34 mmol). MS: m/z 292 [M+1]⁺.

To a solution of Compound 52-7 (99.8 mg, 0.34 mmol) and DIEA (0.2 mL) in toluene (5 mL) was added phosphorus oxychloride (0.3 mL) under nitrogen atmosphere, then the mixture was stirred at 100° C. for 3 hours. Upon completion, the mixture was concentrated under reduced pressure. The residue was diluted with DCM (15 mL) then DIEA (0.2 mL) and 1,4-oxazepane (96 mg, 0.45 mmol) were added. The reaction mixture was stirred at room temperature for 1 hour. Upon completion, the residue was diluted with DCM (30 mL) and water (30 mL) and the organic layer was separated. The organic layer was concentrated under reduced pressure. The residue was purified by silica gel chromatography (eluted with Hex:EtOAc=3:1, v/v) to give Compound 52-8 (124 mg, 0.33 mmol). MS: m/z 375 [M+H]⁺.

To a solution of Compound 52-8 (124 mg, 0.33 mmol) and INT 2 (79 mg, 0.50 mmol) in DMSO (5 mL) was added KF (80 mg, 1.38 mmol). The reaction mixture was purged with nitrogen followed by stirring at 85° C. for 17 hours. The mixture was diluted with EtOAc (30 mL) and water (30 mL), and the organic layer 50 was separated. The organic layer was washed with brine (30 mL), dried over anhydrous Na₂SO₄, filtered and concentrated under reduced pressure. The residue was purified by Pre-TLC (eluted with DCM:MeOH=20:1, v/v) to give Compound 52-9 (82 mg, 0.16 mmol). MS: m/z 498 [M+H]⁺.

To a solution of Compound 52-9 (82 mg, 0.16 mmol), INT 3 (86 mg, 0.16 mmol) and Cs₂CO₃ (191 mg, 0.59 mmol) in toluene (8 mL) and water (2 mL) was added cataCXium A Pd G₃ (13 mg, 0.018 mmol) and the 55 mixture was purged with nitrogen followed by stirring at 100° C. for 18 hours. Upon completion, the reaction mixture was concentrated under reduced pressure. The residue was diluted with EtOAc (30 mL) and water (20 mL) and the organic layer was separated. The combined organic layer was concentrated under reduced pressure. The residue was purified by Pre-TLC (eluted with DCM:MeOH=20:1, v/v) to give Compound 52-10 (59 mg, 0.070 mmol). MS: m/z 848 [M+1].

To a solution of Compound 52-10 (59 mg, 0.070 mmol) in CH₃CN (5 mL) was added HCl/1,4-dioxnae (4 M, 2 mL). The reaction mixture was stirred at room temperature for 1 hour. After completion, the residue was diluted with EtOAc (30 mL) and saturated NaHCO₃ aqueous solution. The organic layer was separated and concentrated under reduced pressure to give Compound 52-11 (69 mg, crude). MS: m/z 804 [M+H]⁺.

To a mixture of Compound 52-11 (69 mg, crude) in DMF (5 mL) was added CsF (185 mg, 1.22 mmol). The mixture was stirred at room temperature for 17 hours. After completion, the mixture was diluted with EtOAc (30 mL) and water (20 mL). The mixture was adjusted to pH 8-9 with saturated NaHCO₃ aqueous solution. The organic layer was separated and concentrated under reduced pressure. The residue was purified by Pre-HPLC (C18 column, A: 0.1% TFA in water, B: CH₃CN, Gradient: 15% B to 45% B in 40 min at a flow rate of 60 mL/min, 230 nm) and freeze-dried to give Compound 52 (20.0 mg, TFA salt, 0.026 mmol). MS: m/z 648 [M+H]⁺.

### Example 53

### 5-ethynyl-6-fluoro-4-(8-fluoro-2-(((2R,7aS)-2-fluorotetrahydro-1H-pyrrolizin-7a(5H)-yl)methoxy)-5-(methylamino)-4-(1,4-oxazepan-4-yl)pyrido[4,3-d]pyrimidin-7-yl)naphthalen-2-ol trifluoroacetic acid (“Compound 53”)

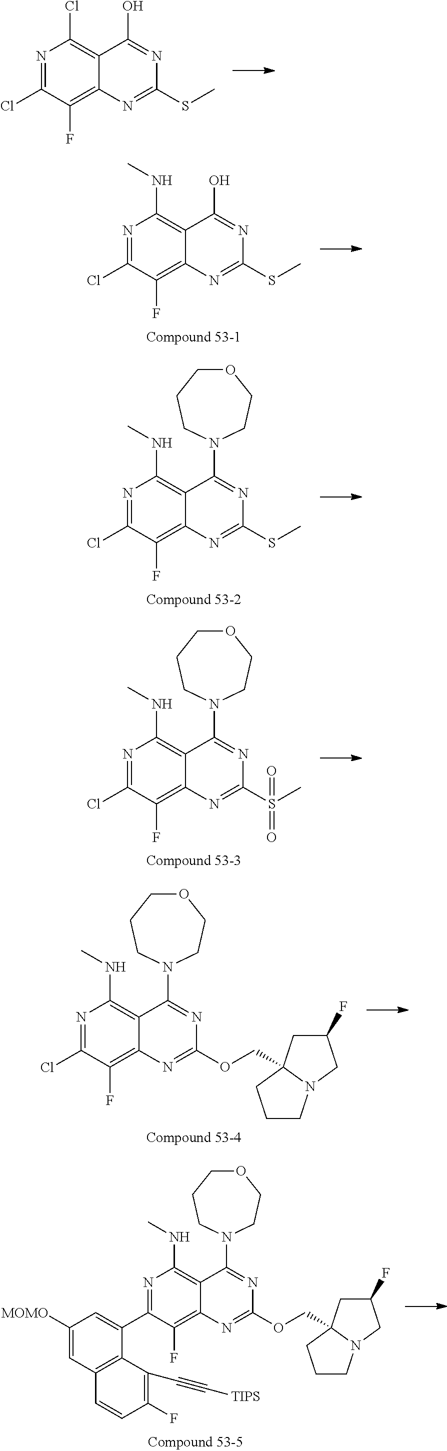

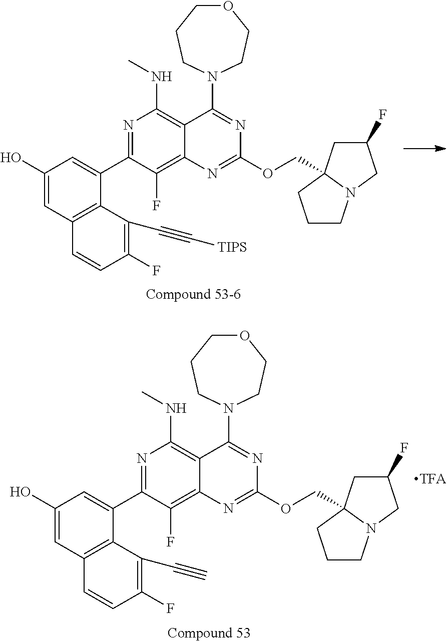

To a mixture of 5,7-dichloro-8-fluoro-2-(methylthio)pyrido[4,3-d]pyrimidin-4-ol (596 mg, 2.13 mmol), Cs₂CO₃ (1117 mg, 3.43 mmol) and DMAc (10 mL) in a 20 mL sealed tube was added methanamine (431 mg, 33% in ethanol (wt. %)). The reaction mixture was stirred at 80° C. for 9 hours. The mixture was diluted with water (15 mL) and the pH value was adjusted to 3 by aqueous hydrochloric acid solution then extracted with EtOAc (15 mL×2). The collected organic layer was washed with brine (20 mL), dried over anhydrous Na₂SO₄ and concentrated under reduced pressure to give Compound 53-1 (536 mg, 1.95 mmol). MS m/z: 275 [M+H]⁺.

To a solution of Compound 53-1 (373 mg, 1.36 mmol) and DIEA (319 mg, 2.47 mmol) in CH₃CN (20 mL) was added POCl₃ (299 mg, 1.95 mmol). The reaction mixture was stirred at 80° C. for 1 hour then concentrated under reduced pressure to give a brown oil. To a solution of 1,4-oxazepane (116 mg, 1.15 mmol) and DIEA (244 mg, 1.89 mmol) in CH₃CN (10 mL) was added a solution of the brown oil in CH₃CN (5 mL). The reaction mixture was stirred at room temperature for 0.5 hour, then diluted with water (30 mL) and extracted with EtOAc (30 mL×2). The collected organic layer was washed with brine (30 mL), dried over anhydrous Na₂SO₄ and concentrated under reduced pressure. The residue was purified by Pre-TLC to give Compound 53-2 (87 mg, 0.24 mmol). MS m/z: 358 [M+H]⁺.

To a solution of Compound 53-2 (56 mg, 0.16 mmol) in DCM (10 mL) was added m-CPBA (99 mg, 0.49 mmol, 85% content). The reaction mixture was stirred at room temperature for 2 hours, quenched with Na₂S₂O₃ aqueous solution (30 mL) and extracted with DCM (30 mL). The organic layer was washed with saturated NaHCO₃ aqueous solution (30 mL), dried over anhydrous Na₂SO₄ and concentrated under reduced pressure to give Compound 53-3 (51 mg, 0.13 mmol). MS m/z: 390 [M+H]⁺.

To a solution of INT 2 (53 mg, 0.33 mmol) in THF (1.5 mL) was added t-BuONa (55 mg, 0.57 mmol) at −30° C. After stirring for 15 min, a solution of Compound 53-3 (51 mg, 0.13 mmol) in THF (1.5 mL) was added to the mixture. The mixture was stirred at room temperature for 0.5 h, then quenched with water (20 mL) and extracted with EtOAc (30 mL). The organic layer was washed with brine (30 mL), dried over anhydrous Na₂SO₄ and concentrated under reduced pressure. The residue was purified by Pre-TLC to give Compound 53-4 (27 mg, 57.58 μmol). MS m/z: 469 [M+H]⁺.

To a solution of Compound 53-4 (27 mg, 57.58 μmol), INT 3 (44 mg, 85.85 μmol) in toluene (4 mL) and water (1 mL) were added Cs₂CO₃ (49 mg, 150.39 μmol) and cataCXium A Pd G₃ (14 mg, 19.22 μmol). The reaction mixture was stirred at 100° C. overnight under nitrogen atmosphere. The mixture was allowed to cool to room temperature, then diluted with water (30 mL) and extracted with EtOAc (2×30 mL). The collected organic layer was washed with NaCl aqueous solution (30 mL), dried over anhydrous Na₂SO₄ and concentrated under reduced pressure. The residue was purified by Pre-TLC to give Compound 53-5 (31 mg, 38.70 μmol). MS m/z: 819 [M+H]⁺.

A solution of Compound 53-5 (31 mg, 38.70 μmol) and HCl (4 M in dioxane, 1 mL) in CH₃CN (3 mL) was stirred at room temperature for 1 hour. The solution was concentrated under reduced pressure. The residue was diluted with saturated NaHCO₃ aqueous solution (50 mL) and extracted with EtOAc (30 mL×2). The collected organic layer was washed with brine (30 mL), dried over anhydrous Na₂SO₄ and concentrated under reduced pressure to give crude Compound 53-6 (crude, 32 mg, 41.29 μmol). MS m/z: 775 [M+H]⁺.

A mixture of crude Compound 53-6 (2 mg, 41.29 μmol) and CsF (0.38 g, 2.50 mmol) in DMF (4 mL) was stirred at 40° C. for 4 hours. The mixture was diluted with water (30 mL) and extracted with EtOAc (30 mL). The organic layer was dried over anhydrous Na₂SO₄ and concentrated under reduced pressure. The residue was purified by Prep-HPLC (C18 column, A: 0.1% TFA in water, B: CH₃CN, Gradient: 15% B to 38% B in 23 min at a flow rate of 40 mL/min, 232 nm) and freeze-dried to give Compound 53 (12.2 mg, 16.65 μmol, TFA salt). MS m/z: 619 [M+H]⁺.

### Example 54

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl methyl(2-(methylamino)ethyl)carbamate trifluoroacetic acid (“Compound 54”)

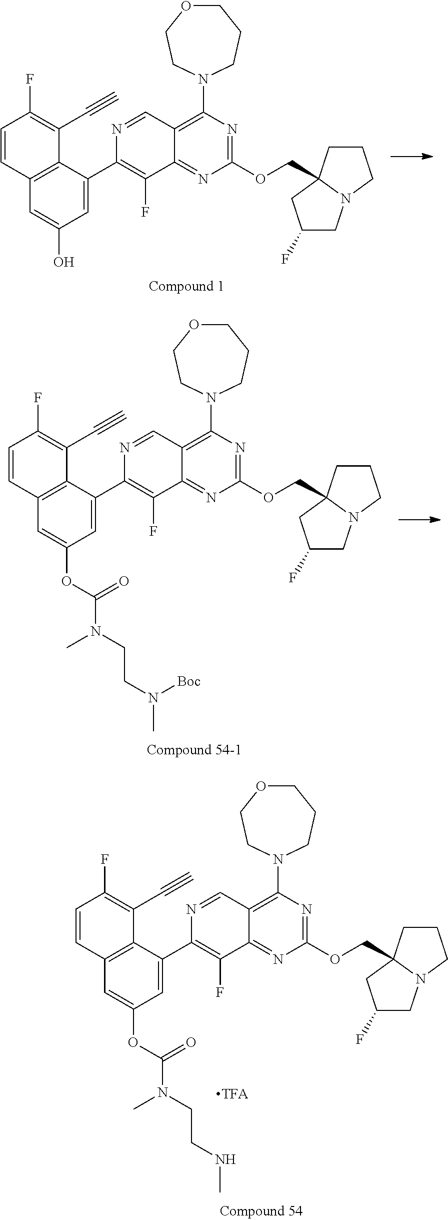 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)N(C)CCN(C)C(=O)OC(C)(C)C)cc(-c3ncc4c(N5CCCOCC5)nc(OC[C@@]56CCCN5C[C@H](F)C6)nc4c3F)c12 ; C#Cc1c(F)ccc2cc(OC(=O)N(C)CCNC)cc(-c3ncc4c(N5CCCOCC5)nc(OC[C@@]56CCCN5C[C@H](F)C6)nc4c3F)c12]

To a solution of tert-butyl methyl(2-(methylamino)ethyl)carbamate (237 mg, 1.26 mmol) and pyridine (138 mg, 1.74 mmol) in DCM (15 mL) was added triphosgene (290 mg, 1.80 mmol) at 0° C. The reaction mixture was stirred at room temperature for 3 hours. The mixture was concentrated under reduced pressure. A solution of the residue in acetonitrile (5 mL) was added dropwise to a mixture of Compound 1 (407 mg, 0.69 mmol) and K₂CO₃ (575 mg, 4.16 mmol) in acetonitrile (15 mL). The reaction mixture was stirred at 80° C. overnight. After being cooled to room temperature, the reaction mixture was diluted with brine (30 mL) and extracted with EtOAc (2×30 mL). The combined organic layer was dried over anhydrous Na₂SO₄ and concentrated under reduced pressure. The residue was purified by Prep-TLC (eluted with MeOH:DCM=1:10, v/v) to give Compound 54-1 (0.73 g, 72.14% yield). MS m/z: 804 [M+H]⁺.

A solution of Compound 54-1 (730 mg, 0.91 mmol) and TFA (3 mL) in DCM (10 mL) was stirred for 2 hours at room temperature. The mixture was concentrated under reduced pressure. The residue was purified by Prep-HPLC (Daisogel C18 column, 50 mm×250 mm, 10 m; A: 0.1% TFA in water, B: CH₃CN, Gradient: 15% B to 32% B in 24 min at a flow rate of 60 mL/min, 230 nm) and freeze-dried to give Compound 54 (0.4101 g, 27.61% yield, TFA salt). MS m/z: 704 [M+H]⁺.

### Example 55

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl 2-phenyl-2-(piperidin-1-yl)acetate (“Compound 55”)

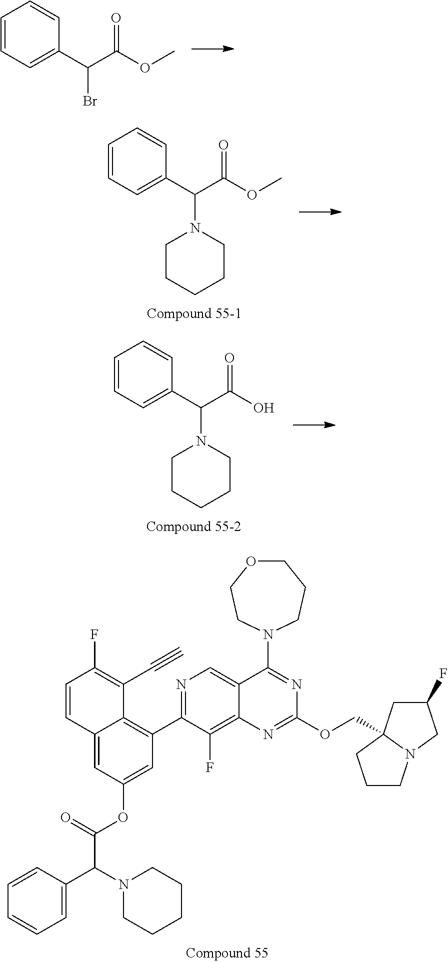 [SMILES: COC(=O)C(Br)c1ccccc1 ; COC(=O)C(c1ccccc1)N1CCCCC1 ; O=C(O)C(c1ccccc1)N1CCCCC1 ; C#Cc1c(F)ccc2cc(OC(=O)C(c3ccccc3)N3CCCCC3)cc(-c3ncc4c(N5CCCOCC5)nc(OC[C@@]56CCCN5C[C@H](F)C6)nc4c3F)c12]

To a solution of methyl 2-bromo-2-phenylacetate (0.36 mL, 2.29 mmol) in acetonitrile (7 mL) were added N,N-diisopropylethylamine (0.43 mL, 2.60 mmol) and piperidine (0.25 mL, 2.29 mmol). The mixture was stirred at room temperature for 2 hours. The reaction mixture was concentrated under reduced pressure and the residue was purified by silica gel column chromatography (eluted with 0-5% EtOAc in hex to give Compound 55-1 (634 mg, 2.71 mmol, 118.43% yield). MS (ESI, m/z): 234 [M+H]⁺.

To a solution of Compound 55-1 (634 mg, 2.71 mmol) in 1,4-dioxane (10 mL) was added concentrated hydrochloric acid (5 mL). The mixture was stirred at 105° C. for 36 hours, then concentrated under reduced pressure to give Compound 55-2 (668 mg, 2.61 mmol, 96.1% yield). MS (ESI, m/z): 220 [M+H]⁺.

A solution of Compound 55-2 (36 mg, 140.76 μmol), Compound 1 (52 mg, 88.19 μmol), DCC (34 mg, 164.78 μmol) and DMAP (15 mg, 122.78 μmol) in DCM (2 mL) was stirred at room temperature for 16 hours. The mixture was quenched with saturated NH₄Cl aqueous solution (30 mL) and extracted with DCM (50 mL). The organic layer was dried over anhydrous Na₂SO₄ and concentrated under reduced pressure. The residue was purified by chiral-HPLC separation using a CHIRALPAK IG column (2 cm×25 cm, 5 um) on a Prep-HPLC-Gilson eluting with (Hex:DCM=3:1)/EtOH=1:1 at a flow rate of 20 mL/min and freeze-dried to give Compound 55 (26.9 mg, 34.01 μmol, 38.5% yield). MS (ESI, m/z): 791 [M+H]⁺.

### Example 56

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl 4-nitrobenzenesulfonate trifluoroacetic acid (“Compound 56”)

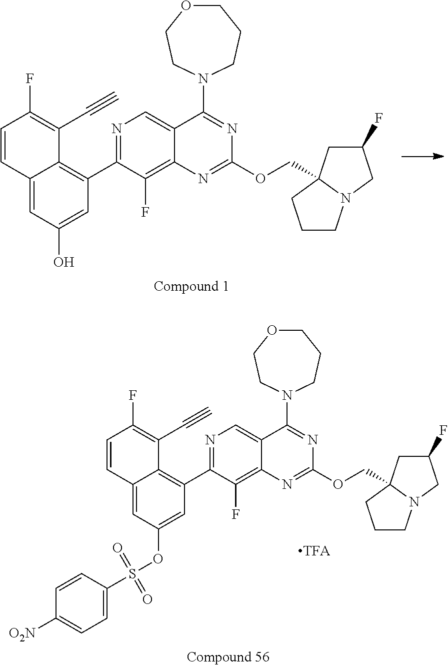 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OS(=O)(=O)c3ccc([N+](=O)[O-])cc3)cc(-c3ncc4c(N5CCCOCC5)nc(OC[C@@]56CCCN5C[C@H](F)C6)nc4c3F)c12]

To a solution of Compound 1 (54 mg, 91.58 μmol) in dichloromethane (3 mL) were added N,N-diisopropylethylamine (24 mg, 185.69 μmol) and 4-nitrobenzenesulfonyl chloride (29 mg, 130.85 μmol). The mixture was stirred at room temperature for 30 min. The mixture was quenched with saturated NH₄Cl aqueous solution (10 mL) and extracted with DCM (2×30 mL). The organic layer was dried over anhydrous Na₂SO₄ and concentrated under reduced pressure. The residue was purified by Prep-HPLC (Daisogel-C18, phase A: 0.1% TFA in water, phase B: CH₃CN, Gradient: 15% B to 50% B in 40 min at a flow rate of 60 mL/min, 235 nm) and freeze-dried to give Compound 56 (61.1 mg, 68.74 μmol, TFA salt, 75.1% yield). MS (ESI, m/z): 775 [M+H]⁺.

### Example 57

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl ethylcarbamate trifluoroacetic acid (“Compound 57”)

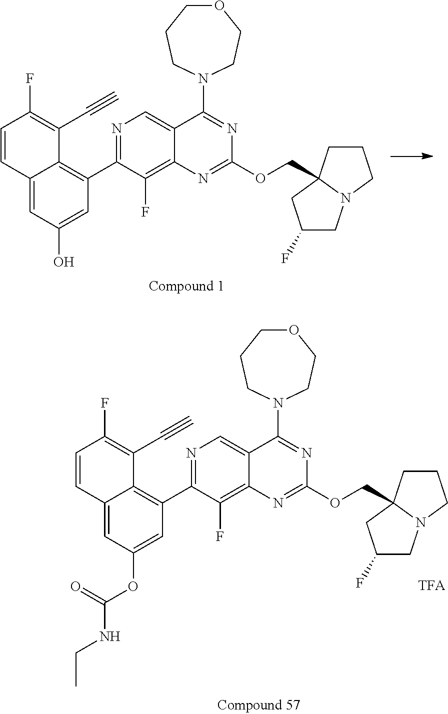 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)NCC)cc(-c3ncc4c(N5CCCOCC5)nc(OC[C@@]56CCCN5C[C@H](F)C6)nc4c3F)c12]

To a solution of Compound 1 (321 mg, 0.54 mmol) in THF (4 mL) was added isocyanatoethane (268 mg, 3.77 mmol). The mixture was stirred at 80° C. for 16 hours. The mixture was concentrated under reduced pressure. The residue was purified by Pre-HPLC (C18 column, A: 0.05% TFA in water, B: CH₃CN, Gradient: 15% B to 45% B in 60 min at a flow rate of 60 mL/min, 230 nm) and freeze-dried to give Compound 57 (301.2 mg, TFA salt, 0.34 mmol). MS: m/z 661 [M+H]⁺.

### Example 58

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl 2-(dimethylamino)-2-phenylpropanoate trifluoroacetic acid (“Compound 58”)

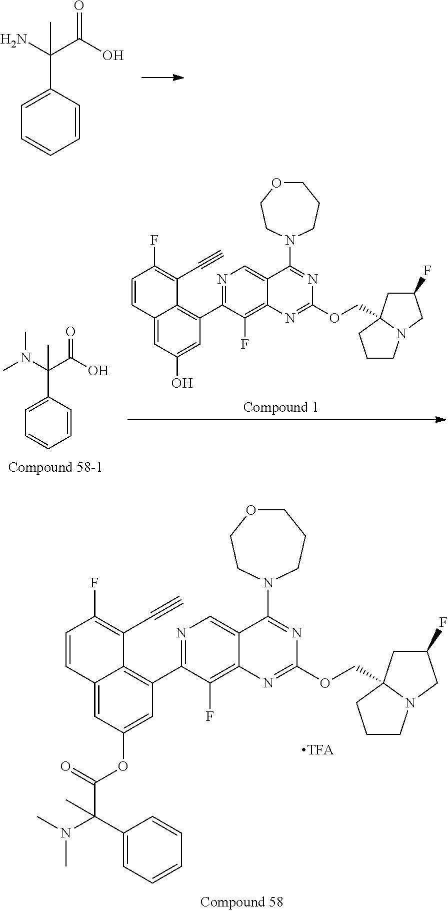 [SMILES: CC(N)(C(=O)O)c1ccccc1 ; CN(C)C(C)(C(=O)O)c1ccccc1 ; C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)C(C)(c3ccccc3)N(C)C)cc(-c3ncc4c(N5CCCOCC5)nc(OC[C@@]56CCCN5C[C@H](F)C6)nc4c3F)c12]

To a solution of 2-amino-2-phenylpropanoic acid (501 mg, 3.03 mmol) in methanol (10 mL) were added formaldehyde (1.5 mL), HCl (1 N, 1.5 mL) and Pd/C (0.36 g). The reaction mixture was stirred at room temperature for 16 hours under hydrogen atmosphere. The resulting mixture was filtered and the filtrate was concentrated under reduced pressure. The residue was purified by reverse phase flash (eluted with 5-20% CH₃CN in H₂O (0.1% TFA)) to give Compound 58-1 (844 mg, 2.74 mmol, 90.5% yield). MS (ESI, m/z): 194 [M+H]⁺.

A solution of Compound 58-1 (50 mg, 162.72 μmol), Compound 1 (52 mg, 88.19 μmol), EDCI (33 mg, 172.14 μmol), HOBT (26 mg, 192.41 μmol), DMAP (9 mg, 73.66 μmol) and N,N-Diisopropylethylamine (63 mg, 487.45 μmol) in DCM (2 mL) was stirred at room temperature for 16 hours. The mixture was quenched with saturated NH₄Cl aqueous solution (30 mL) and extracted with DCM (50 mL). The organic layer was dried over anhydrous Na₂SO₄ and concentrated under reduced pressure. The residue was purified by Prep-HPLC (C18 column, phase A: 0.1% TFA in water, phase B: CH₃CN, Gradient: 15% B to 30% B in 18 min at a flow rate of 60 mL/min, 230 nm) and freeze-dried to give Compound 58 (15 mg, 17.06 μmol, TFA salt, 19.3% yield). MS (ESI, m/z): 765 [M+H]⁺.

### Example 59

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl 2-(dimethylamino)-2-phenylacetate trifluoroacetic acid (“Compound 59”)

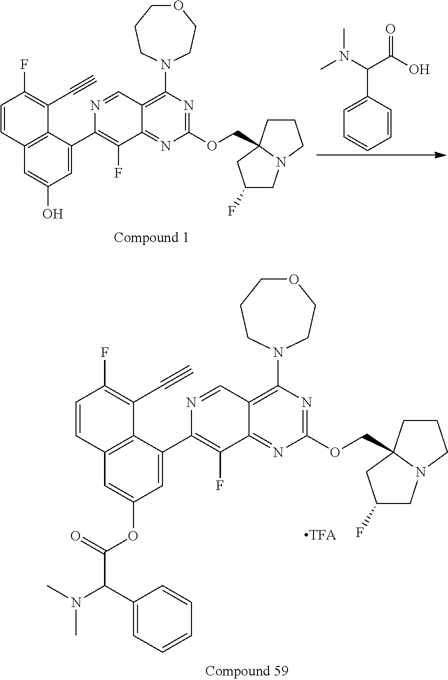

To a solution of 2-(dimethylamino)-2-phenylacetic acid (18 mg, 100.44 μmol), DCC (29 mg, 140.55 μmol) and DMAP (16 mg, 130.97 μmol) in DCM (2 mL) was added Compound 1 (49 mg, 83.11 μmol). The reaction mixture was stirred at 60° C. overnight. The reaction mixture was diluted with water (10 mL) and extracted with DCM (2×10 mL). The organic layers were combined, dried over anhydrous Na₂SO₄ and concentrated under reduced pressure. The residue was purified by Prep-HPLC (C18 column, A: 0.1% TFA in water, B: CH₃CN, Gradient: 15% B to 34% B in 35 min at a flow rate of 40 mL/min, 230 nm) and freeze-dried to give Compound 59 (25.2 mg, 29.14 μmol, TFA salt). MS m/z: 751 [M+H]⁺.

### Example 60

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl L-valinate trifluoroacetic acid (“Compound 60”)

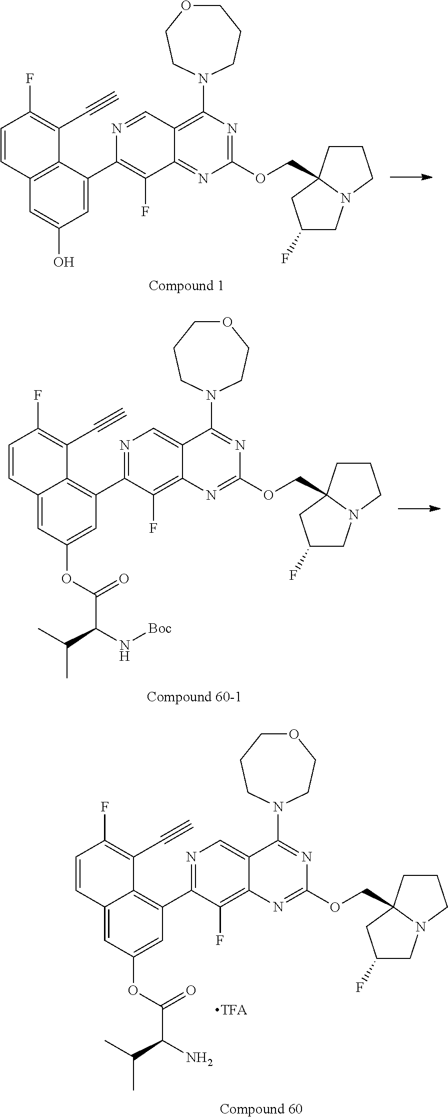 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)[C@@H](NC(=O)OC(C)(C)C)C(C)C)cc(-c3ncc4c(N5CCCOCC5)nc(OC[C@@]56CCCN5C[C@H](F)C6)nc4c3F)c12 ; C#Cc1c(F)ccc2cc(OC(=O)[C@@H](N)C(C)C)cc(-c3ncc4c(N5CCCOCC5)nc(OC[C@@]56CCCN5C[C@H](F)C6)nc4c3F)c12]

A mixture of (tert-butoxycarbonyl)-L-valine (45 mg, 0.21 mmol), Compound 1 (78 mg, 0.13 mmol), DMAP (26 mg, 0.21 mmol) and DCC (58 mg, 0.28 mmol) in DCM (10 mL) was stirred at room temperature for 3 hours. The mixture was filtered and the filtrate was diluted with water (20 mL). The separated organic layer was dried over anhydrous Na₂SO₄ and concentrated under reduced pressure to give Compound 60-1 (187 g, crude). MS m/z: 789 [M+H]⁺.

A solution of Compound 60-1 (730 mg, 0.91 mmol) and TFA (2 mL) in DCM (5 mL) was stirred for 2.5 hours at room temperature. The mixture was concentrated under reduced pressure. The residue was purified by Prep-HPLC (YMC-Triart C18-S12 nm column, 50 mm×250 mm, 7 m; A: 0.05% TFA in water, B: CH₃CN, Gradient: 10% B to 40% B in 28 min to 60% B in 30 min at a flow rate of 70 mL/min, 250 n) and freeze-dried to give Compound 60 (76.4 mg, 20.07% yield, TFA salt). MS m/z: 689 [M+H]⁺.

### Example 61

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl dimethylglycinate trifluoroacetic acid (“Compound 61”)

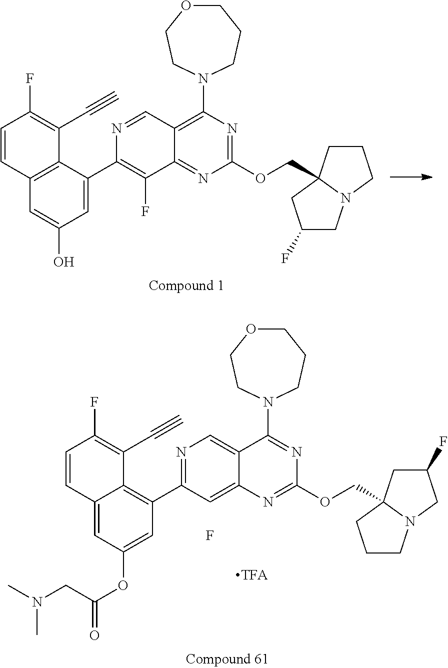

A mixture of Compound 1 (49 mg, 0.083 mmol), dimethylglycine (25 mg, 0.24 mmol), HOBT (13 mg, 0.096 mmol), EDCI (19 mg, 0.099 mmol) and DIEA (110 mg, 0.85 mmol) in DMF (5 mL) was stirred for 3 hours at room temperature. The mixture was diluted with water (20 mL) and extracted with EtOAc (2×20 mL). The combined organic layer was dried over anhydrous Na₂SO₄, filtered and concentrated under reduced pressure. The residue was purified by Prep-HPLC (Daisogel C18 column, 50 mm×250 mm, 10 m; A: 0.1% TFA in water, B: CH₃CN, Gradient: 15% B to 35% B in 30 min at a flow rate of 60 mL/min, 230 nm) and freeze-dried to give Compound 61 (44.8 mg, 34.17% yield, TFA salt). MS m/z: 675 [M+H]⁺.

### Example 62

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl (R)-4-((3R,5S,7R,8R,9S,10S,12S,13R,14S,17R)-3,7,12-trihydroxy-10,13-dimethylhexadecahydro-1H-cyclopenta[a]phenanthren-17-yl)pentanoate trifluoroacetic acid (“Compound 62”)

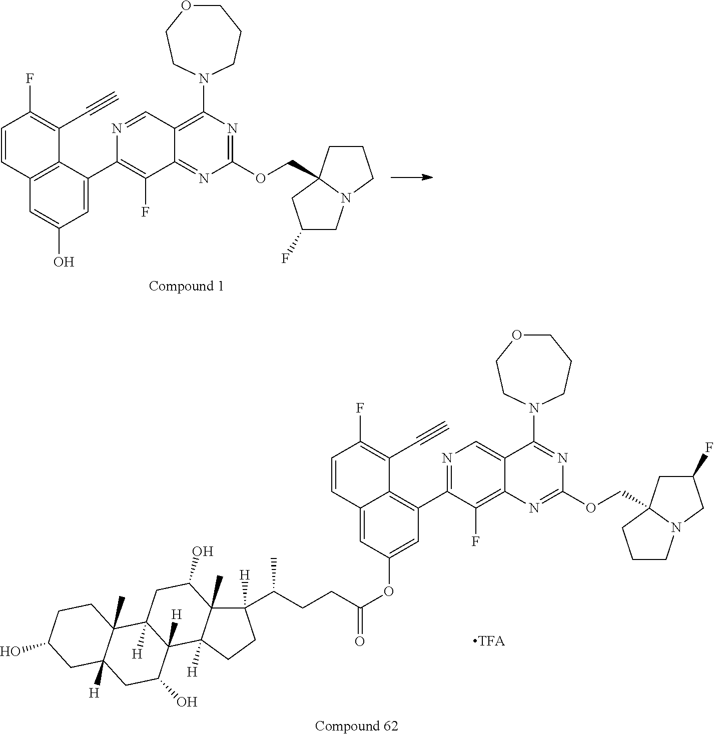

A mixture of Compound 1 (50 mg, 0.085 mmol), cholic acid (49 mg, 0.012 mmol), HOBT (24 mg, 0.17 mmol), EDCI (41 mg, 0.21 mmol) and DIEA (61 mg, 0.047 mmol) in DMF (5 mL) was stirred for 7 hours at room temperature. The mixture was diluted with water (20 mL) and extracted with EtOAc (2×20 mL). The combined organic layer was dried over anhydrous Na₂SO₄, filtered and concentrated under reduced pressure. The residue was purified by Prep-HPLC (Daisogel C18 column, 50 mm×250 mm, 10 m; A: 0.1% TFA in water, B: CH₃CN, Gradient: 15% B to 64% B in 47 min to 64% B in 4 min at a flow rate of 60 mL/min, 230 nm) and freeze-dried to give Compound 62 (63.1 mg, 68.00% yield, TFA salt). MS m/z: 981 [M+H]⁺.

### Example 63

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl 5-((3aS,4S,6aR)-2-oxohexahydro-1H-thieno[3,4-d]imidazol-4-yl)pentanoate trifluoroacetic acid (“Compound 63”)

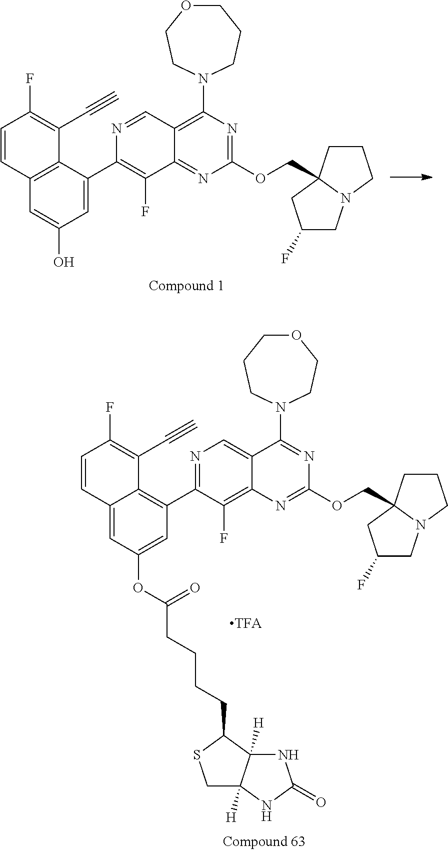 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)CCCC[C@@H]3SC[C@@H]4NC(=O)N[C@@H]43)cc(-c3ncc4c(N5CCCOCC5)nc(OC[C@@]56CCCN5C[C@H](F)C6)nc4c3F)c12]

A mixture of Compound 1 (54 mg, 0.092 mmol), D-biotin (58 mg, 0.24 mmol), HOBT (24 mg, 0.17 mmol), EDCI (34 mg, 0.18 mmol) and DIEA (78 mg, 0.60 mmol) in DMF (5 mL) was stirred for 7 hours at room temperature. The mixture was diluted with water (20 mL) and extracted with EtOAc (2×20 mL). The combined organic layer was dried over anhydrous Na₂SO₄, filtered and concentrated under reduced pressure. The residue was purified by Prep-HPLC (YMC-Triart C18-S12 nm column, 50 mm×250 mm, 7 m; A: 0.05% TFA in water, B: CH₃CN, Gradient: 20% B to 45% B in 28 min to 65% B in 30 min at a flow rate of 70 mL/min, 238 nm) and freeze-dried to give Compound 63 (56.6 mg, 66.46% yield, TFA salt). MS m/z: 816 [M+H]⁺.

### Example 64

### 5-ethynyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol trifluoroacetic acid (“Compound 64”)

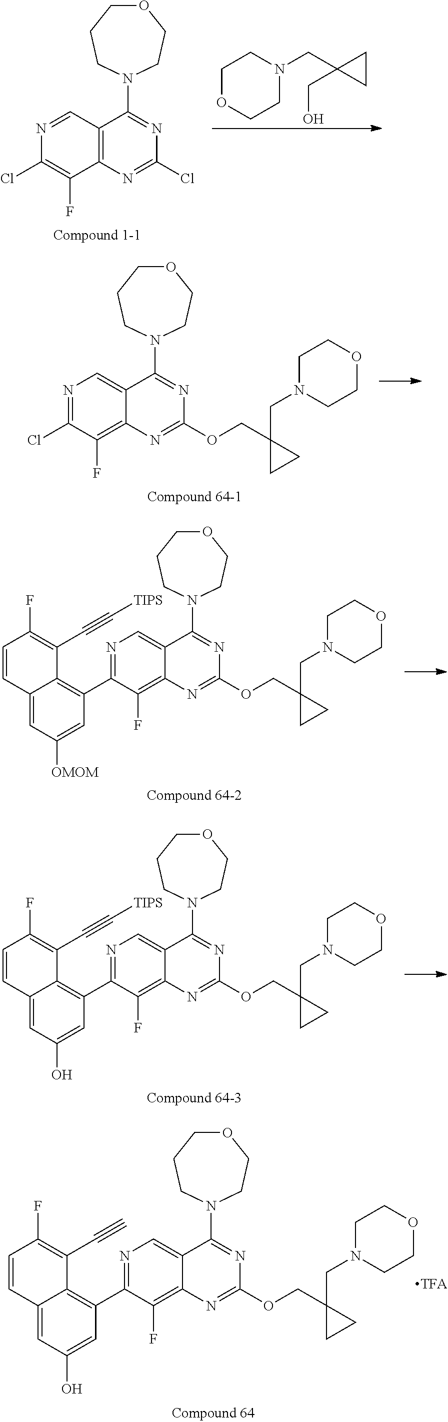

A solution of Compound 1-1 (131 mg, 413.0591 μmol), (1-(morpholinomethyl)cyclopropyl)methanol (95 mg, 554.7896 μmol) and KF (79 mg, 1.3598 mmol) in DMSO (8 mL) was stirred at 100° C. for 20 h under nitrogen atmosphere. The mixture was allowed to cool to room temperature, diluted with saturated NaHCO₃ aqueous solution (20 mL) and extracted with EA (2×20 mL). The organic layer was washed with 20 mL aqueous NaCl solution, then dried over anhydrous Na₂SO₄ and concentrated in vacuum. The residue was purified by Pre-TLC to give Compound 64-1 (160 mg, 354.0441 μmol, 85.7127% yield). MS: m/z: 452 [M+H]⁺.

Compound 64-2 and Compound 64-3 are prepared successively by similar steps in the Example 1.

A solution of Compound 64-3 (0.19941 g, 263.0801 μmol) and CsF (224 mg, 1.4746 mmol) in DMF (5 mL) was stirred for 20 h at 40° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 35 min at a flow rate of 60 mL/min, 240 nm) to afford the desired product Compound 64 (31 mg, 51.5257 μmol, TFA salt, 19.5855% yield). MS: m/z: 602 [M+H]⁺.

### Example 65

### 5-ethynyl-6-fluoro-4-(8-fluoro-2-(((2R,7aS)-2-fluorotetrahydro-1H-pyrrolizin-7a(5H)-yl)methoxy)-5-methoxy-4-(1,4-oxazepan-4-yl)pyrido[4,3-d]pyrimidin-7-yl)naphthalen-2-ol trifluoroacetic acid (“Compound 65”)

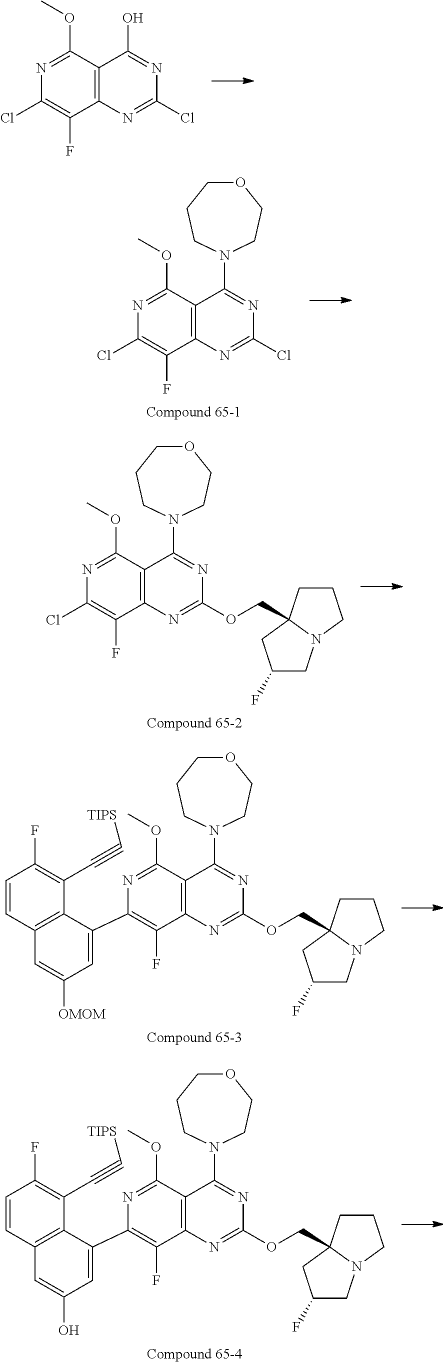

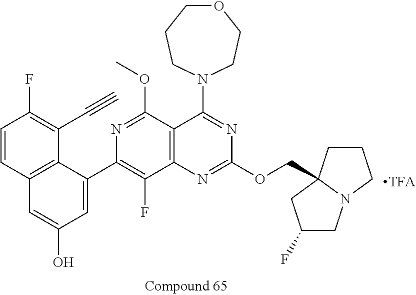 [SMILES: C#Cc1c(F)ccc2cc(O)cc(-c3nc(OC)c4c(N5CCCOCC5)nc(OC[C@@]56CCCN5C[C@H](F)C6)nc4c3F)c12]

To a solution of 2,7-dichloro-8-fluoro-5-methoxypyrido[4,3-d]pyrimidin-4-ol (76.1 mg, 0.29 mmol) and DIEA (0.2 mL) in toluene (5 mL) was added phosphorus oxychloride (0.5 mL) under nitrogen and then the mixture was stirred at 100° C. for 3 h. Upon completion, the mixture was concentrated under reduced pressure. The residue was diluted with DCM (15 mL), then DIEA (0.1 mL) and 1,4-oxazepane (41 mg, 0.40 mmol) was added and the reaction mixture was stirred at room temperature for 1 h. Upon completion, the residue was diluted with DCM (30 mL) and water (30 mL). The organic layer was separated and concentrated under reduced pressure. The residue was purified by silica gel chromatography (eluting with Hex:EA=3:1, v/v) to give Compound 65-1 (158 mg, 0.46 mmol). MS: m/z 347 [M+H]⁺.

To a solution of Compound 65-1 (158 mg, 0.46 mmol) and INT 2 (106 mg, 0.67 mmol) in DMSO (6 mL) was added KF (106 mg, 1.82 mmol), then the mixture was purged with N₂ followed by stirring at 85° C. for 24 h. The mixture was diluted with EA (30 mL) and water (30 mL) and the organic layer was separated. The organic layer was washed with brine (20 mL), dried over anhydrous Na₂SO₄, filtered and concentrated under reduced pressure. The residue was purified by Pre-TLC (DCM:MeOH=15:1) to give Compound 65-2 (47 mg, 0.21 mmol). MS: m/z 470 [M+H]⁺.

To a solution of Compound 65-2 (47 mg, 0.10 mmol), INT 3 (79 mg, 0.15 mmol) and Cs₂CO₃ (102 mg, 0.31 mmol) in toluene (10 mL) and water (2 mL) was added cataCXium A Pd G3 (7 mg, 0.0096 mmol) and the mixture was purged with N₂ followed by stirring at 100° C. for 16 h. Upon completion, the mixture was concentrated under reduced pressure and the residue was diluted with EA (30 mL) and water (20 mL). The organic layer was separated. The combined organic layer was concentrated under reduced pressure. The residue was purified by Pre-TLC (DCM:MeOH=15:1) to give Compound 65-3 (78 mg, 0.95 mmol). MS: m/z 820 [M+1]⁺.

To a solution of Compound 65-3 (78 mg, 0.95 mmol) in CH₃CN (5 mL) was added HCl (4 M in 1,4-dioxane, 1.5 mL). The reaction mixture was stirred at 0° C. for 1 h. After completion, the residue was diluted with EA (30 mL) and saturated NaHCO₃ aqueous solution. The organic layer was separated and concentrated under reduced pressure to give Compound 65-4 (85 mg, 0.11 mmol). MS: m/z 776 [M+H]⁺.

To a mixture of Compound 65-4 (85 mg, 0.11 mmol) in DMF (5 mL) was added CsF (234 mg, 1.54 mmol). The mixture was stirred at room temperature for 16 h. After completion, the mixture was diluted with EA (30 mL) and water (20 mL) and the mixture was adjusted to pH=8-9 with saturated NaHCO₃ aqueous solution. The organic layer was separated and concentrated under reduced pressure. The residue was purified by Pre-HPLC (C18 column, A: 0.1% TFA in water, B: CH₃CN, Gradient: 15% B to 40% B in 30 min at a flow rate of 60 mL/min, 230 nm) to give Compound 65 (52.6 mg, 0.072 mmol, TFA salt). MS: m/z 620 [M+H]⁺.

### Example 66

### 4-(5-ethoxy-8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)-5-ethynyl-6-fluoronaphthalen-2-ol (“Compound 66”)

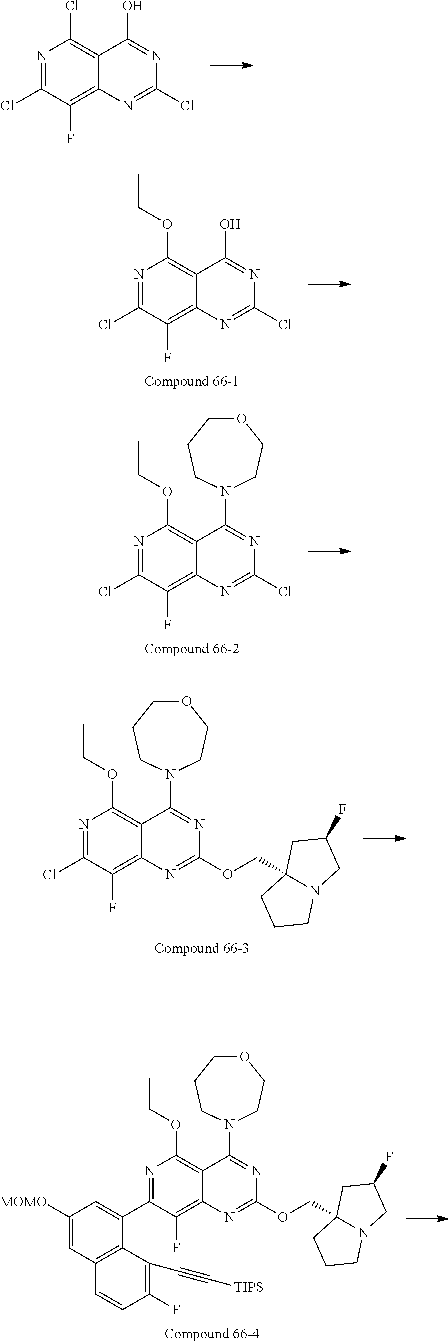

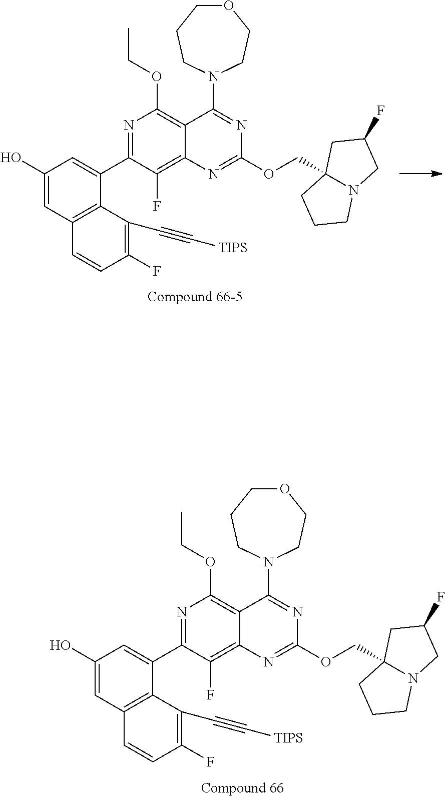

To a solution of ethanol (85 mg, 1.85 mmol) in THF (15 mL) was added NaH (196 mg, 4.90 mmol, 60%) at 0° C. and the mixture was stirred for 10 min, then 2,5,7-trichloro-8-fluoropyrido[4,3-d]pyrimidin-4-ol (485 mg, 1.85 mmol) was added. The mixture was stirred at 0° C. for 17 h. The resulting mixture was quenched by water (5 mL) and the pH of the mixture was adjusted to 3. The mixture was extracted with EA (30 mL×2). The organic layer was washed with brine (30 mL), dried over anhydrous Na₂SO₄ and concentrated in vacuum to give the desired product Compound 66-1 (575 mg, 2.07 mmol). MS (ESI, m/z): 278 [M+H]⁺.

To a solution of Compound 66-1 (382 mg, 1.37 mmol) in toluene (10 mL) were added DIEA (1 mL) and POCl₃ (1.5 mL) and the mixture was stirred for 3 h at 100° C. The reaction mixture was concentrated under vacuum. The residue was dissolved in DCM (10 mL) and added to a solution of 1,4-oxazepane (128 mg, 1.27 mmol) and DIEA (680 mg, 5.26 mmol) in DCM (10 mL) at −5° C. The mixture was stirred at room temperature for 0.5 h. The reaction was diluted with H₂O (30 mL) and extracted with DCM (30 mL). The organic layer was dried over anhydrous Na₂SO₄ and concentrated under vacuum. The residue was purified by Pre-TLC to give the desired product Compound 66-2 (231 mg, 0.64 mmol). MS (ESI, m/z): 361 [M+H]⁺.

To a solution of Compound 66-2 (231 mg, 0.64 mmol) and INT 2 (109 mg, 0.68 mmol) in DMSO (10 mL) was added KF (117 mg, 2.01 mmol). The reaction mixture was stirred at 95° C. for 17 h under nitrogen atmosphere. The resulting mixture was quenched by water (30 mL) and extracted with EA (2×30 mL). The organic layer was washed with brine (30 mL), dried over anhydrous Na₂SO₄ and concentrated in vacuum. The residue was purified by Pre-TLC to afford Compound 66-3 (95 mg, 0.20 mmol). MS (ESI, m/z): 484 [M+H]⁺.

A solution of Compound 66-3 (95 mg, 0.20 mmol), INT 3, (126 mg, 0.25 mmol), cataCXium A Pd G₃ (22 mg, 30.21 μmol), Cs₂CO₃ (136 mg, 0.42 mmol) in toluene (6 mL) and water (1.5 mL) was stirred at 100° C. for 20 h under nitrogen atmosphere. The reaction was diluted with EA (30 mL) and washed with water (2×20 mL). The organic layer was dried over anhydrous Na₂SO₄ and concentrated under vacuum. The residue was purified by Pre-TLC to afford Compound 66-4 (144 mg, 172.65 μmol). MS (ESI, m/z): 834 [M+H]⁺.

To a solution of Compound 66-4 (144 mg, 172.65 μmol) in CH₃CN (3 mL) was added HCl (1 mL, 4 M in 1,4-dioxane). The reaction mixture was stirred at room temperature for 1 h. The resulting mixture was quenched by saturated NaHCO₃ aqueous solution (20 mL) and extracted with EA (2×30 mL). The organic layer was dried over anhydrous Na₂SO₄ and concentrated under reduced pressure to afford Compound 66-5 (134 mg, 169.62 μmol). MS (ESI, m/z): 790 [M+H]⁺.

To a solution of Compound 66-5 (134 mg, 169.62 μmol) in DMF (5 mL) was added CsF (0.36 g, 2.37 mmol) and the reaction mixture was stirred at 40° C. for 17 h. The mixture was diluted with saturated aqueous NaHCO₃ solution (20 mL) and extracted with EA (2×20 mL). The organic layers were combined, dried over anhydrous Na₂SO₄ and concentrated under vacuum. The residue was purified by Prep-HPLC (C18 column, A: 0.1% TFA in water, B: CH₃CN, Gradient: 15% B to 35% B in 37 min at a flow rate of 60 mL/min, 230 nm). The eluent was adjusted to pH 8 and the acetonitrile in the eluent was concentrated. The resulting aqueous phase was extracted with EA (30 mL×2), and the organic phase was dried and concentrated then freeze-dried to afford Compound 66 (25.7 mg, 40.56 μmol). MS (ESI, m/z): 634 [M+H]⁺.

### Example 67

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl methylcarbamate trifluoroacetic acid (“Compound 67”)

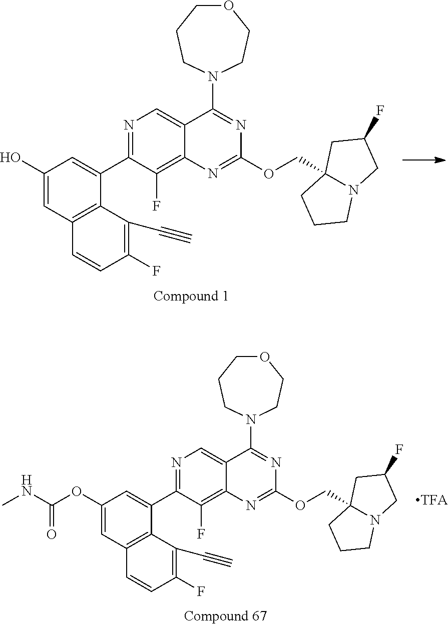 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)NC)cc(-c3ncc4c(N5CCCOCC5)nc(OC[C@@]56CCCN5C[C@H](F)C6)nc4c3F)c12]

To a suspension of Compound 1 (48 mg, 81.41 gmol) and DIEA (36 mg, 0.28 mmol) in DCM (10 mL) was added methylcarbamic chloride (17 mg, 0.18 mmol). The reaction mixture was stirred at room temperature for 5 h. The reaction mixture was concentrated under vacuum. The residue was purified by Pre-HPLC (C18 column, A: 0.1% TFA in water, B: CH₃CN, Gradient: 15% B to 40% B in 37 min at a flow rate of 60 mL/min, 230 nm) to give Compound 67 (12.4 mg, 16.30 gmol, TFA salt). MS m/z: 647 [M+H]⁺.

### Example 68

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl benzenesulfonate trifluoroacetic acid (“Compound 68”)

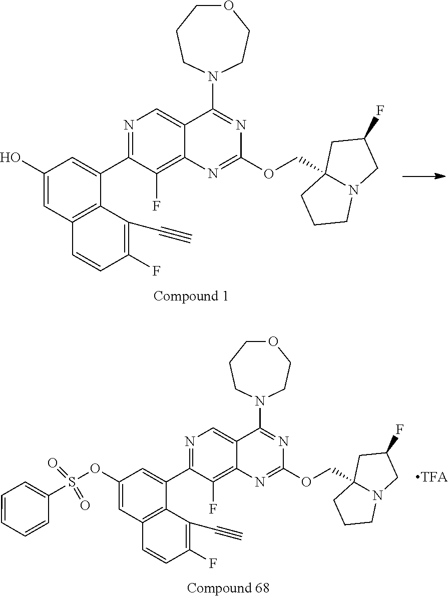 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OS(=O)(=O)c3ccccc3)cc(-c3ncc4c(N5CCCOCC5)nc(OC[C@@]56CCCN5C[C@H](F)C6)nc4c3F)c12]

To a solution of Compound 1 (50 mg, 84.80 gmol) and DIEA (4 drops) in DCM (10 mL) was added benzenesulfonyl chloride (2 drops). The reaction mixture was stirred at room temperature for 1 h. The reaction mixture was concentrated under vacuum. The residue was purified by Pre-HPLC (C18 column, A: 0.1% TFA in water, B: CH₃CN, Gradient: 15% B to 54% B in 52 min at a flow rate of 60 mL/min, 230 nm) to give Compound 68 (61.8 mg, 73.24 gmol, TFA salt). MS m/z: 730 [M+H]⁺.

### Example 69

### (S)-4-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methyl-1,4-oxazepane trifluoroacetic acid (“Compound 69”)

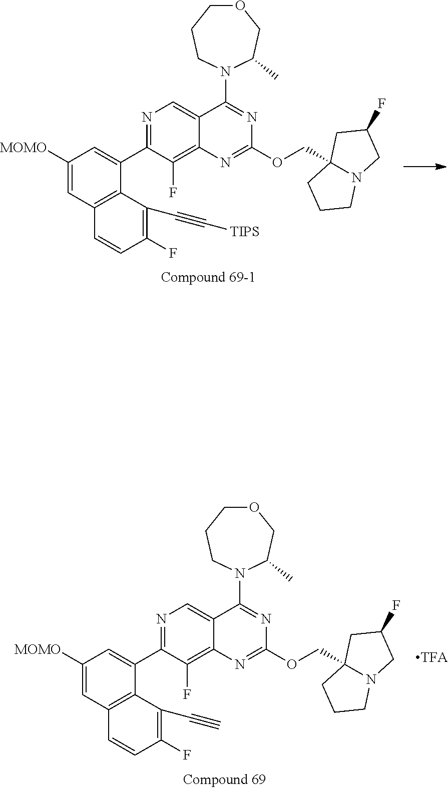

To a solution of Compound 69-1 (0.25 g, 0.31 mmol) in DMF (5 mL) was added CsF (0.40 g, 2.63 mmol). The reaction mixture was stirred overnight at 40° C. The mixture was diluted with saturated aqueous NaHCO₃ solution (20 mL) and extracted with EA (2×20 mL). The organic layers were combined, dried over anhydrous Na₂SO₄ and concentrated under vacuum. The residue was purified by Prep-HPLC (C18 column, A: 0.1% TFA in water, B: CH₃CN, Gradient: 15% B to 47% B in 36 min at a flow rate of 60 mL/min, 235 nm) to afford the desired product Compound 69 (193.7 mg, 0.25 mmol, TFA salt). MS m/z: 648 [M+H]⁺.

### Example 70

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl 2-amino-6-methylbenzoate trifluoroacetic acid (“Compound 70”)

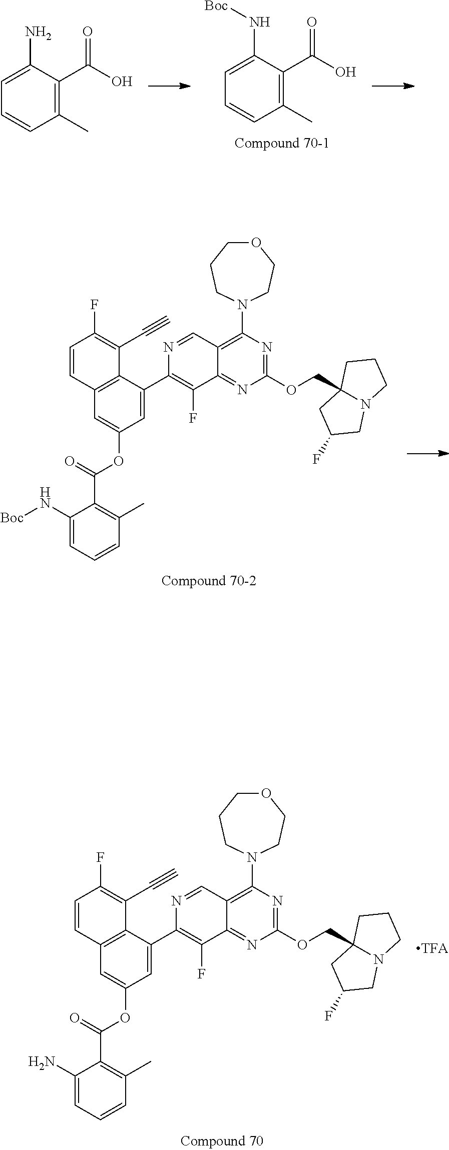 [SMILES: Cc1cccc(N)c1C(=O)O ; Cc1cccc(NC(=O)OC(C)(C)C)c1C(=O)O ; C#Cc1c(F)ccc2cc(OC(=O)c3c(C)cccc3NC(=O)OC(C)(C)C)cc(-c3ncc4c(N5CCCOCC5)nc(OC[C@@]56CCCN5C[C@H](F)C6)nc4c3F)c12 ; C#Cc1c(F)ccc2cc(OC(=O)c3c(C)cccc3N)cc(-c3ncc4c(N5CCCOCC5)nc(OC[C@@]56CCCN5C[C@H](F)C6)nc4c3F)c12]

To a solution of 2-amino-6-methylbenzoic acid (1.01 g, 6.68 mmol) and TEA (0.75 g, 7.41 mmol) in ACN (12 mL) was added dropwise di(tert-butyl) carbonate (1.53 g, 7.01 mmol). The reaction mixture was stirred at room temperature for 2 h. The reaction was diluted with water (50 mL) and extracted with EA (2×50 mL). The organic layers were combined and washed with 50 mL 1N HCl. The organic layer was dried over anhydrous Na₂SO₄ and concentrated under vacuum to give Compound 70-1 (1.52 g, 6.05 mmol). MS m/z: 250 [M−H]⁻.

To a solution of Compound 70-1 (32 mg, 127.35 μmol), DCC (34 mg, 164.79 μmol), DMAP (8 mg, 65.48 μmol) in DCM (2 mL) was added Compound 1 (72 mg, 122.12 μmol). The reaction mixture was stirred overnight at 60° C. The reaction was diluted with water (10 mL) and extracted with DCM (2×10 mL). The organic layers were combined and dried over anhydrous Na₂SO₄ and concentrated under vacuum. The residue was purified by Pre-TLC (DCM:MeOH=10:1, v/v) to give the desired product Compound 70-2 (111 mg, 134.89 μmol). MS m/z: 823 [M+H]⁺.

A solution of Compound 70-2 (111 mg, 134.89 μmol) and TFA (1 mL) in DCM (3 mL) was stirred at room temperature for 1 h. The mixture was concentrated in vacuum. The residue was diluted with saturated aqueous NaHCO₃ solution (10 mL) and extracted with EA (2×10 mL). The organic layers were combined, dried over anhydrous Na₂SO₄ and concentrated under vacuum. The residue was purified by Prep-HPLC (C18 column, A: 0.1% TFA in water, B: CH₃CN, Gradient: 15% B to 55% B in 58 min at a flow rate of 60 mL/min, 230 nm) to afford the desired product Compound 70 (65.6 mg, 78.40 μmol, TFA salt). MS m/z: 723 [M+H]⁺.

### Example 71

### ethyl N-(((5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl)oxy)carbonyl)-N-methylglycinate trifluoroacetic acid (“Compound 71”)

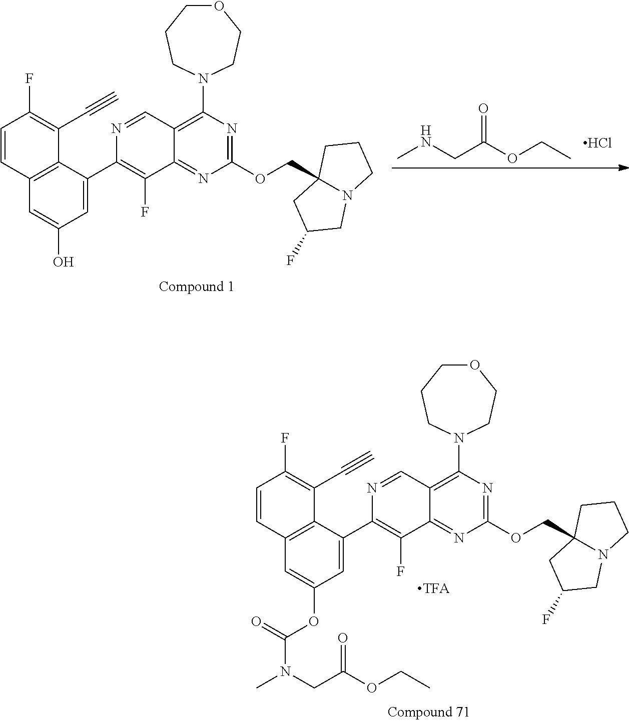

To a solution of pyridine (84 mg, 1.06 mmol) in DCM (3 mL) was added portionwise triphosgene (79 mg, 266.22 μmol) at 0° C. The reaction mixture was stirred at room temperature for 20 minutes. Then ethyl methylglycinate hydrochloride (43 mg, 279.94 μmol) was added and the mixture was stirred at room temperature for 2 h. The reaction was concentrated under vacuum and the residue was dissolved in pyridine (2 mL). Compound 1 (102 mg, 173.00 μmol) was added to this mixture mol. The reaction mixture was stirred at 106° C. overnight under nitrogen atmosphere. The reaction was quenched with water (10 mL) and extracted with EA (2×20 mL). The organic layers were combined and washed with 10 mL 1N HCl, 10 mL 1N NaHCO₃, 20 mL brine successively, dried over anhydrous Na₂SO₄ and concentrated under vacuum. The residue was purified by Prep-HPLC (C18 column, A: 0.05% TFA in water, B: CH₃CN, Gradient: 15% B to 75% B in 60 min at a flow rate of 60 mL/min, 240 nm) to afford the desired product Compound 71 (20.1 mg, 23.74 μmol, TFA salt). MS m/z: 733 [M+H]⁺.

### Example 72

### 1-(5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl) 2-methyl piperidine-1,2-dicarboxylate trifluoroacetic acid (“Compound 72”)

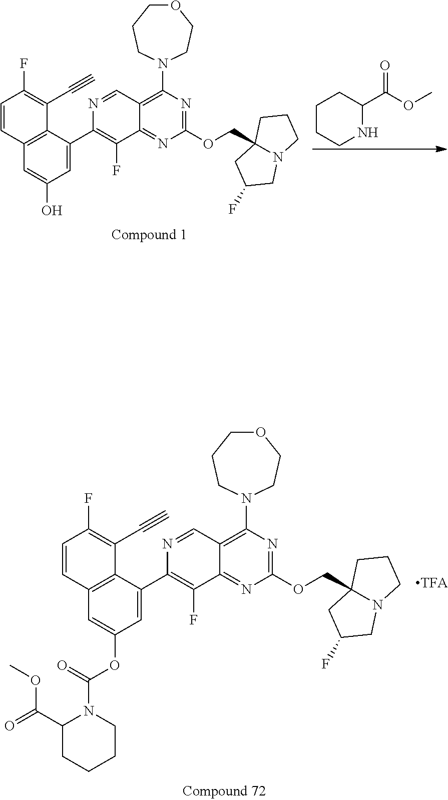

To a solution of pyridine (269 mg, 3.40 mmol) in DCM (6 mL) was added portionwise triphosgene (101 mg, 340.36 μmol) at 0° C. The reaction mixture was stirred at room temperature for 20 minutes. Then methyl 2-piperidinecarboxylate (62 mg, 433.01 μmol) was added and the mixture was stirred at room temperature for 2 h. The reaction was concentrated under vacuum and the residue was dissolved in pyridine (2.5 mL). Then Compound 1 (205 mg, 347.69 μmol) was added to the mixture. The reaction mixture was stirred at 106° C. overnight under nitrogen atmosphere. The reaction was quenched with water (10 mL) and extracted with EA (2×20 mL). The organic layers were combined and washed with 10 mL 1N HCl, 10 mL 1N NaHCO₃ and 20 mL brine successively, and then dried over anhydrous Na₂SO₄ and concentrated under vacuum. The residue was purified by Prep-HPLC (C18 column, A: 0.1% TFA in water, B: CH₃CN, Gradient: 15% B to 55% B in 60 min at a flow rate of 60 mL/min, 230 nm) to afford the desired product Compound 72 (21.7 mg, 24.86 μmol, TFA salt). MS m/z: 759 [M+H]⁺.

### Example 73

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl pivalate trifluoroacetic acid (“Compound 73”)

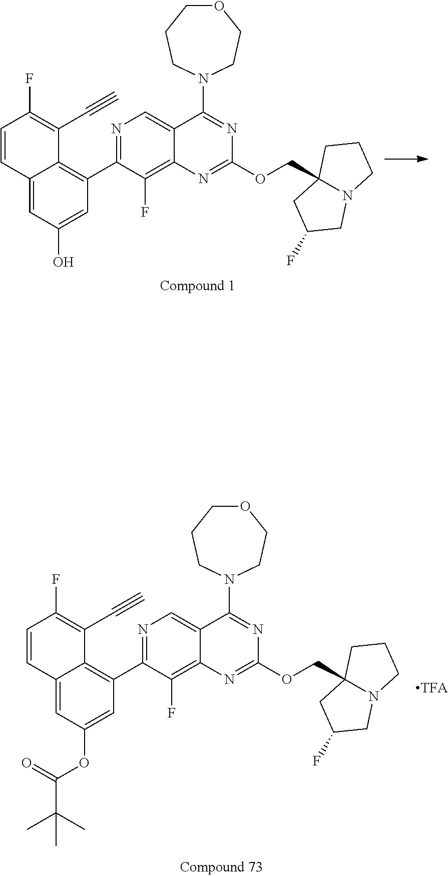 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)C(C)(C)C)cc(-c3ncc4c(N5CCCOCC5)nc(OC[C@@]56CCCN5C[C@H](F)C6)nc4c3F)c12]

To a solution of Compound 1 (40.7 mg, 0.069 mmol) and triethylamine (0.2 mL) in DCM (20 mL) was added pivaloyl chloride (1 drop), then the mixture was stirred at room temperature for 1 h. After completion, the reaction mixture was concentrated under reduced pressure. The residue was purified by Pre-HPLC (C18 column, A: 0.1% TFA in water, B: CH₃CN, Gradient: 15% B to 65% B in 60 min at a flow rate of 60 mL/min, 230 nm) to give Compound 73 (47.3 mg, 0.060 mmol, TFA salt). MS: m/z 674 [M+H]⁺.

### Example 74

### 5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl adamantane-1-carboxylate trifluoroacetic acid (“Compound 74”)

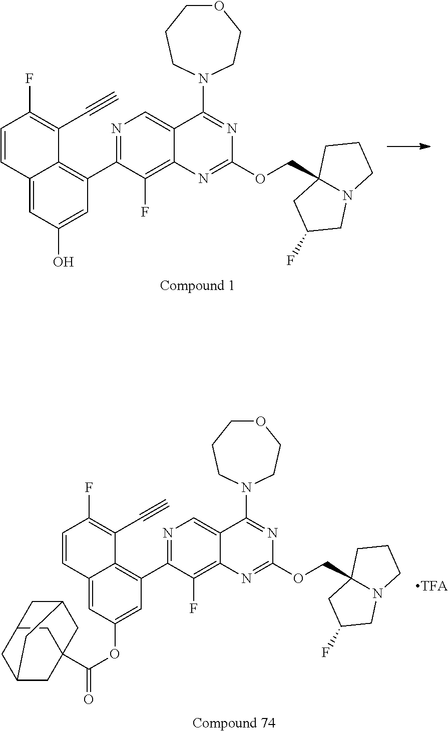 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OC(=O)C34CC5CC(CC(C5)C3)C4)cc(-c3ncc4c(N5CCCOCC5)nc(OC[C@@]56CCCN5C[C@H](F)C6)nc4c3F)c12]

To a solution of Compound 1 (46.2 mg, 0.078 mmol), 1,3-dicyclohexylcarbodiimide (41.0 mg, 0.199 mmol) and 4-dimethylaminopyridin (2.4 mg, 0.020 mmol) in DCM (3 mL) was added 1-adamantanecarboxylic acid (30.8 mg, 0.17 mmol). The reaction mixture was stirred at room temperature for 20 h. After completion, the reaction mixture was concentrated under reduced pressure. The residue was purified by Pre-HPLC (C18 column, A: 0.1% TFA in water, B: CH₃CN, Gradient: 15% B to 90% B in 60 min at a flow rate of 60 mL/min, 230 nm) to give Compound 74 (55.0 mg, 0.064 mmol, TFA salt). MS: m/z 752 [M+H]⁺.

### Example 75

### diethyl (5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl) phosphate trifluoroacetic acid (“Compound 75”)

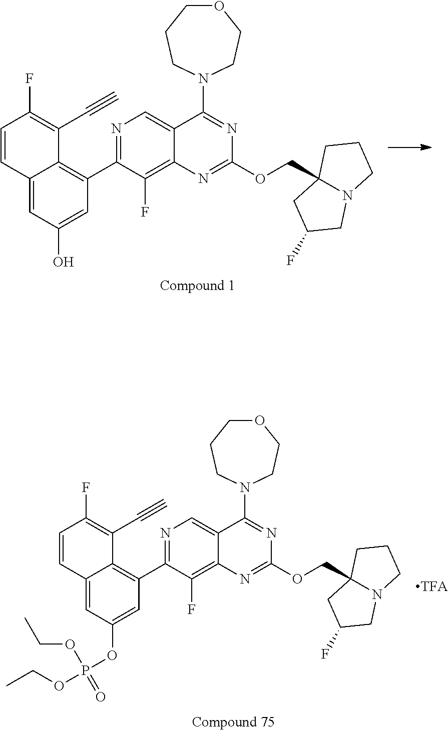 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OP(=O)(OCC)OCC)cc(-c3ncc4c(N5CCCOCC5)nc(OC[C@@]56CCCN5C[C@H](F)C6)nc4c3F)c12]

To a solution of triethyl phosphate (76 mg, 0.42 mmol) and trifluoromethanesulfonic anhydride (178 mg, 0.63 mmol) in DCM (8 mL) was added pyridine (74 mg, 0.94 mmol). The mixture was stirred at room temperature for 10 min. Then Compound 1 (118 mg, 0.20 mmol) was added. The reaction mixture was stirred at room temperature for 16 h. After completion, the reaction mixture was diluted with DCM (50 mL), water (30 mL) and the organic layer was separated. The organic layer was washed with brine (30 mL) and dried over anhydrous Na₂SO₄, filtered and concentrated under reduced pressure. The residue was purified by Pre-HPLC (C18 column, A: 0.1% TFA in water, B: CH₃CN, Gradient: 15% B to 65% B in 60 min at a flow rate of 60 mL/min, 240 nm) to give Compound 75 (73.3 mg, 0.087 mmol, TFA salt). MS: m/z 726 [M+H]⁺.

### Example 76

### ((5-ethynyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-yl)oxy)methyl pivalate (“Compound 76”)

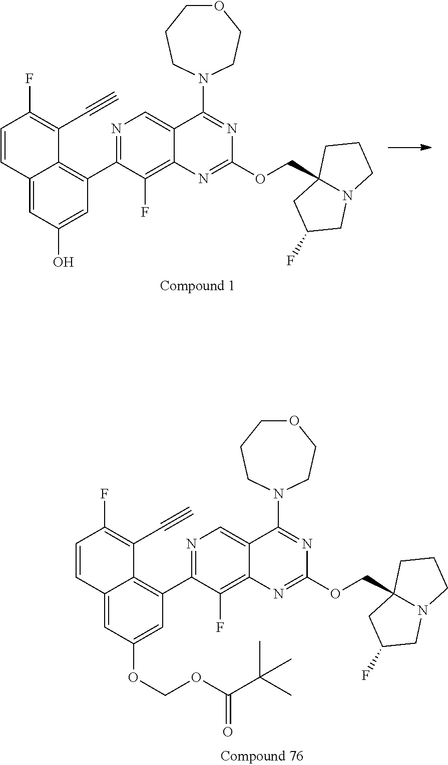 [SMILES: C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(OCOC(=O)C(C)(C)C)cc(-c3ncc4c(N5CCCOCC5)nc(OC[C@@]56CCCN5C[C@H](F)C6)nc4c3F)c12]

To a solution of Compound 1 (99.5 mg, 0.17 mmol) in DMF (10 mL) was added sodium hydride (21.6 mg, 0.54 mmol, 60% content) under nitrogen at 0° C. and then stirred at room temperature for 30 minutes. Then the solution of iodomethyl pivalate (86 mg, 0.36 mmol) in DMF (1 mL) was added to the reaction mixture and stirred at room temperature for 30 minutes. After completion, the reaction mixture was added to 1% citric acid aqueous solution (40 mL) and extracted with EA (50 mL). The organic layer was separated and concentrated under reduced pressure. The residue was purified by Pre-HPLC (C18 column, A: 0.1% TFA in water, B: CH₃CN, Gradient: 15% B to 35% B in 35 min at a flow rate of 60 mL/min, 230 nm), the crude was separated by Prep-HPLC-Gilson with the following conditions: Column, CHIRAL ART Cellulose-SC column (2 cm×25 cm, 5 um), mobile phase, Hex(0.1% DEA):EtOH=50:50; Flowing rate: 20 mL/min to give Compound 76 (70.5 mg, 0.12 mmol). MS: m/z 704 [M+H]⁺.

### Example 77

### 4-(4-((1S,7R)-5-oxa-2-azabicyclo[5.1.0]octan-2-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 77A or Compound 77B”)

### 4-(4-((1R,7S)-5-oxa-2-azabicyclo[5.1.0]octan-2-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 77B or Compound 77A”)

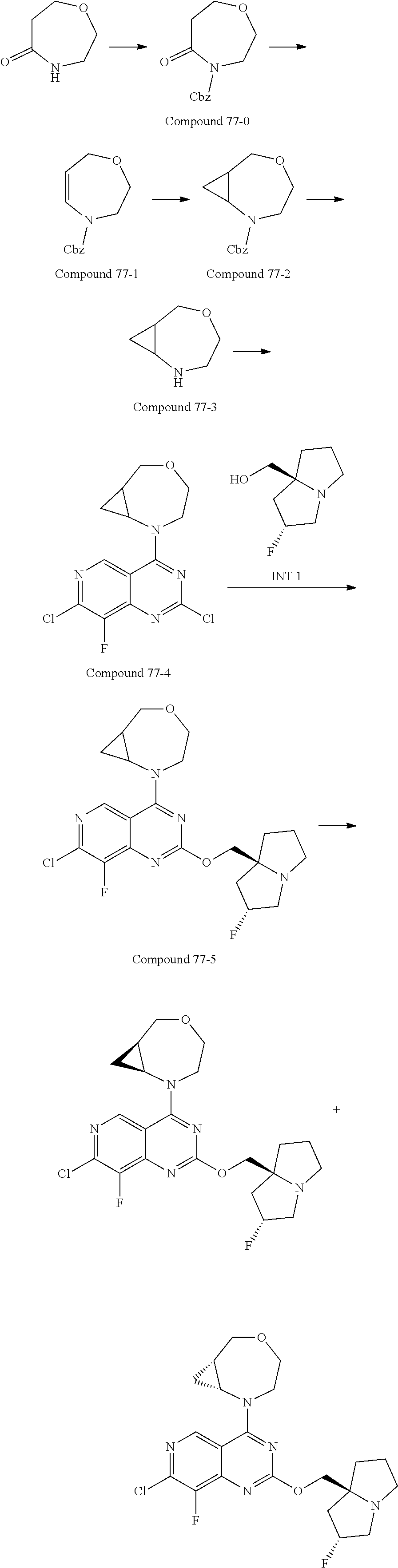

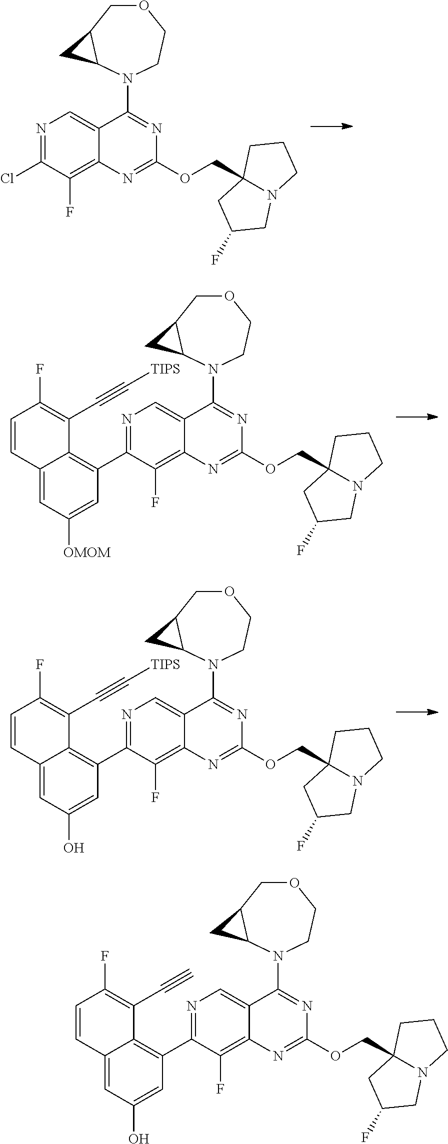

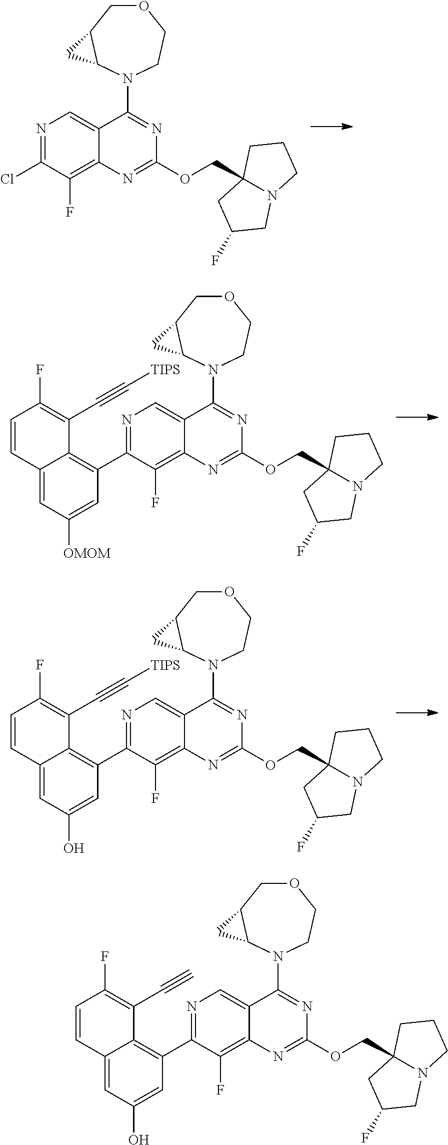

To a solution of 1,4-oxazepan-5-one (1.05 g, 9.1201 mmol) in THF (15 mL) was added n-BuLi (3.8 mL) at −78° C. The mixture was stirred at −78° C. for 30 min. CbzCl (1.56 g, 9.1446 mmol) was added to the solution at −78° C. and stirred 1 h. The solution was quenched by saturated NH₄Cl aqueous solution, extracted with EA, dried over Na₂SO₄ and concentrated in vacuum. The residue was purified by C18 column eluted with H₂O/ACN (10%-100%, 30 min) to afford Compound 77-0 (0.88 g, 3.5304 mmol). MS: m/z: 250 [M+1]⁺.

Lithium triethylborohydride (3 mL) was added to a solution of benzyl 5-oxo-1,4-oxazepane-4-carboxylate (0.50 g, 2.0059 mmol) in toluene (10 mL) at −78° C. and the mixture was stirred at −78° C. for 2 h. DMAP (0.30 g, 2.4557 mmol), DIEA (1.49 g, 11.5287 mmol), trifluoroacetic anhydride (3.2 g, 15.2358 mmol) were added at −78° C. The solution was stirred at 25° C. for 12 h. The reaction mixture was adjusted to pH=7 with saturated NaHCO₃ aqueous solution and extracted with EA (2×15 mL). The combined organic layer was washed with saturated NaCl (2×15 mL) aqueous solution, dried over Na₂SO₄ and concentrated in vacuum. The residue was purified by C18 column eluted with H₂O/ACN (10%-100%, 30 min) to afford Compound 77-1 (0.39 g, 1.6719 mmol). MS: m/z: 234 [M+1]⁺.

To a solution of Compound 77-1 (0.71 g, 3.0438 mmol) in DCM (15 mL) was add ZnEt₂ (1 M in hex, 13 mL) at 20° C. and the mixture was stirred at 20° C. for 0.5 h. A solution of MeI₂ (4.10 g, 15.3080 mmol) in DCM (15 mL) was added at 0° C. and the solution was stirred at 25° C. for 3 h. The reaction was quenched with saturated NH₄Cl (15 mL) aqueous solution and extracted with EA (2×15 mL). The combined organic layer was washed with brine (3×15 mL), dried over Na₂SO₄ and concentrated in vacuum. The residue was purified by column chromatography (SiO₂, EA/Hex=0%-100%) to give Compound 77-2 (656 mg, 2.6528 mmol). MS: m/z: 248 [M+1]⁺.

A solution of 77-2 (0.40 g, 1.6175 mmol), Pd/C (0.76 g) and di-tert-butyl dicarbonate (1.01 g, 4.6278 mmol) in EA (10 mL) was stirred at room temperature in H₂ atmosphere for 12 h. The mixture was filtered and washed with EA (2×10 mL). The filtrate was concentrated under vacuum. The residue was added to a solution of TFA (1 mL) and DCM (5 mL), stirred for 1 h at room temperature and concentrated under reduced pressure. The crude product Compound 77-3 (183 mg, crude) is not purified and used for the next step directly. MS: m/z: 114 [M+1]⁺.

A solution of 2,4,7-trichloro-8-fluoropyrido[4,3-d]pyrimidine (0.40 g, 1.5844 mmol), Compound 77-3 (183 mg, crude) and DIEA (0.47 g, 3.64 mmol) in DCM (10 mL) was stirred at 0° C. for 1.5 h. The solution was extracted with DCM (2×10 mL). The combined organic layer was washed with saturated NaCl (10 mL) aqueous solution, dried over Na₂SO₄ and concentrated in vacuum. The residue was purified by column chromatography (EA/Hex=0%-100%) to give Compound 77-4 (513 mg, 1.5585 mmol). MS: m/z: 329 [M+1]⁺.

A solution of Compound 77-4 (513 mg, 1.5585 mmol), INT 2 (0.42 g, 2.6384 mmol) and KF (0.46 g, 7.9178 mmol) in DMSO (10 mL) was stirred at 100° C. for 20 h. The mixture was cooled to room temperature and diluted with saturated NaCl (15 mL) aqueous solution and extracted with EA (2×15 mL). The combined organic layer was washed with aqueous NaCl (15 mL) solution, dried over Na₂SO₄ and concentrated in vacuum. The residue was purified by Pre-TLC to give Compound 77-5 (0.33 g, 730.2559 μmol). MS: m/z: 452 [M+1]⁺.

The Compound 77-5 was separated by Prep-HPLC-Gilson with the following conditions: Column, CHIRALPAK-IG column (2 cm×25 cm, 5 um); mobile phase (Hex:DCM=1:1) (0.1% diethylamine)/EtOH(50:50); Flowing rate: 20 mL/min. This results in Compound 77-6A (102 mg, the first eluting isomer, Retention Time 5.731 min) and Compound 77-6B (113 mg, the second eluting isomer, Retention Time 6.693 min).

A solution of Compound 77-6A (102 mg, 225.7155 μmol), INT 3 (152 mg, 296.5653 μmol), cataCXium A Pd G3 (87 mg, 119.4612 μmol) and cesium carbonate (223 mg, 684.4293 μmol) in toluene (10 mL) and water (2 mL) was stirred at 100° C. overnight under nitrogen atmosphere. The mixture was cooled to room temperature, diluted with saturated NaCl (15 mL) aqueous solution and extracted with DCM (2×15 mL). The combined organic layer was washed with saturated NaCl aqueous solution (15 mL), dried over Na₂SO₄ and concentrated in vacuum. The residue was purified by Pre-TLC to give Compound 77-7A (151 mg, 188.2771 mol). MS: m/z: 802 [M+1]⁺.

HCl (4 M in 1,4-dioxane, 1 mL) was added to a solution of Compound 77-7A (151 mg, 188.2771 μmol) in DCM (9 mL) and the mixture was stirred at room temperature for 1 h. The mixture was diluted with 10% NaHCO₃ solution (20 mL) and extracted with DCM (2×15 mL). The combined organic layer was washed with saturated NaCl aqueous solution (15 mL), dried over Na₂SO₄ and concentrated in vacuum to give crude Compound 77-8A (153 mg, crude). MS: m/z: 758 [M+1]⁺.

A solution of Compound 77-8A (153 mg, 201.8585 μmol) and CsF (814 mg, 5.36 mmol) in DMF (10 mL) was stirred for 20 h at 40° C. under nitrogen atmosphere. The solution was diluted with H₂O (10 mL) and extracted with EA (2×10 mL). The combined organic layer was dried over Na₂SO₄ and concentrated under vacuum. The residue was purified by Prep-HPLC (C18 column, phase A: 0.05% NH₄OH in water, phase B: CH₃CN, Gradient: 30% B to 70% B in 34 min at a flow rate of 70 mL/min, 240 nm) to give Compound 77A (65.2 mg, 108.3746 μmol). MS: m/z: 602 [M+1]⁺.

A solution of Compound 77-6B (113 mg, 250.0573 μmol), INT 3 (200 mg, 390.2176 μmol), cataCXium A Pd G3 (96 mg, 131.8192 μmol) and cesium carbonate (264 mg, 810.2661 μmol) in toluene (10 mL) and water (2 mL) was stirred at 100° C. overnight under nitrogen atmosphere. The mixture was cooled to room temperature, diluted with saturated NaCl aqueous solution (15 mL) and extracted with EA (2×15 mL). The combined organic layer was washed with saturated NaCl aqueous solution (15 mL), dried over Na₂SO₄ and concentrated in vacuum. The residue was purified by Pre-TLC to give Compound 77-7B (64 mg, 79.7996 mol). MS: m/z: 802 [M+1]⁺.

HCl (4 M in 1,4-dioxane, 1 mL) was added to a solution of Compound 77-7B (64 mg, 79.7996 μmol) in DCM (9 mL) and the mixture was stirred at room temperature for 1 h. The mixture was diluted with 10% NaHCO₃ solution (20 mL) and extracted with DCM (2×15 mL). The combined organic layer was washed with saturated NaCl aqueous solution (15 mL), dried over Na₂SO₄ and concentrated in vacuum to give crude Compound 77-8B (75 mg, crude). MS: m/z: 758 [M+1]⁺.

A solution of Compound 77-8B (75 mg, crude) and CsF (382 mg, 2.5148 mmol) in DMF (10 mL) was stirred for 20 h at 40° C. under nitrogen atmosphere. The solution was diluted with H₂O (10 mL) and extracted with EA (2×10 mL). The combined organic layer was dried over Na₂SO₄ and concentrated under vacuum. The residue was purified by Prep-HPLC (C18 column, phase A: 0.05% NH₄OH in water, phase B: CH₃CN, Gradient: 30% B to 70% B in 34 min at a flow rate of 70 mL/min, 240 nm) to give Compound 77B (22.4 mg, 37.2330 μmol). MS: m/z: 602 [M+1]⁺.

### Example 78

### 4-(4-(2-oxa-6-azabicyclo[5.1.0]octan-6-yl)-8-fluoro-2-(((2R,7aS)-2-fluorotetrahydro-1H-pyrrolizin-7a(5H)-yl)methoxy)-5-methoxypyrido[4,3-d]pyrimidin-7-yl)-5-ethynyl-6-fluoronaphthalen-2-ol (“Compound 78”)

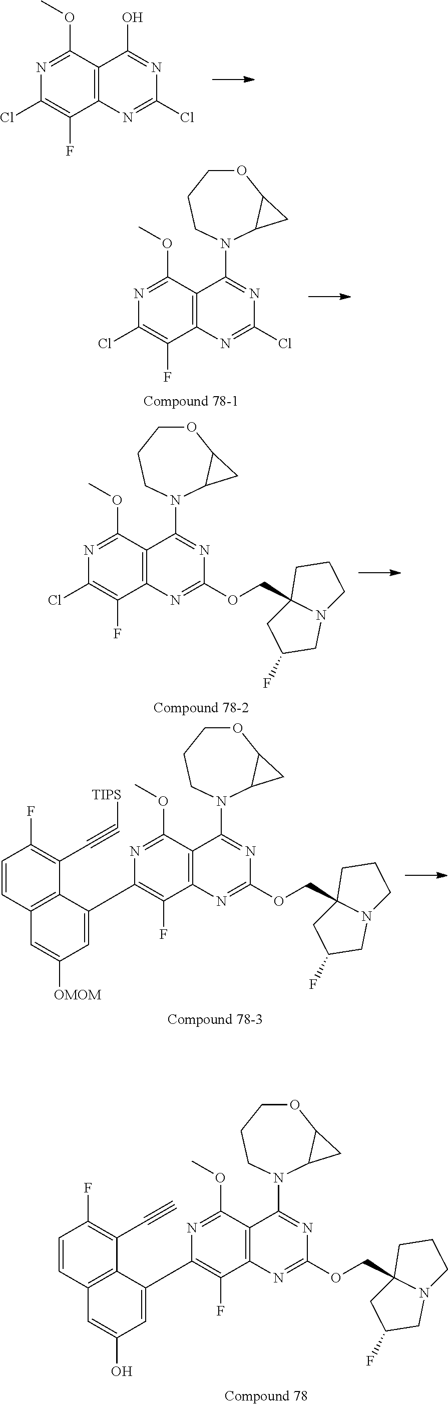

To a solution of 2,7-dichloro-8-fluoro-5-methoxypyrido[4,3-d]pyrimidin-4-ol (130 mg, 0.49 mmol) and DIEA (3 mL) in toluene (10 mL) was added phosphorus oxychloride (3 mL), then the mixture was stirred at 100° C. for 0.5 hour. Upon completion, the mixture was concentrated under reduced pressure. The residue was diluted with DCM (20 mL), then DIEA (2 mL) and Compound 7-5 (158 mg, 1.40 mmol) were added and the reaction mixture was stirred at room temperature for 16 hours. The mixture was concentrated under reduced pressure. The residue was diluted with saturated aqueous NaHCO₃ and extracted with EtOAc (2×30 mL). The organic layers were combined, dried over anhydrous Na₂SO₄, filtered and concentrated under reduced pressure. The residue was purified by silica gel chromatography to give Compound 78-1 (72 mg, 40.71% yield). MS: m/z 359 [M+H]⁺.

To a solution of Compound 78-1 (72 mg, 0.20 mmol) and INT 2 (52 mg, 0.33 mmol) in DMSO (5 mL) was added KF (108 mg, 1.86 mmol). The mixture was stirred at 90° C. for 4 hours. After being cooled to room temperature, the mixture was diluted with water (20 mL) and extracted with EtOAc (2×30 mL). The organic layers were combined, dried over anhydrous Na₂SO₄, filtered and concentrated under reduced pressure. The residue was purified by Pre-TLC (eluted with DCM:MeOH=15:1, v/v) to obtain Compound 78-2 (62 mg, 64.18% yield). MS: m/z 482 [M+H]⁺.

To a solution of Compound 78-2 (62 mg, 0.13 mmol), INT3 (86 mg, 0.17 mmol) and Cs₂CO₃ (150 mg, 0.46 mmol) in toluene (4 mL) and water (1 mL) was added cataCXium A Pd G₃ (20 mg, 0.027 mmol). The mixture was purged with nitrogen followed by stirring at 100° C. for 16 hours. Upon completion, the reaction mixture was diluted with water (20 mL) and extracted with EtOAc (2×20 mL). The organic layers were combined, dried over anhydrous Na₂SO₄, filtered and concentrated under reduced pressure. The residue was purified by Pre-TLC (eluted with DCM:MeOH=10:1, v/v) to obtain Compound 78-3 (20.7 mg, 19.34% yield). MS: m/z 832 [M+1]⁺.

To a solution of Compound 78-3 (30 mg, 0.036 mmol) in acetonitrile (5 mL) was added HCl (4 M in 1,4-dioxane, 1.5 mL). The reaction mixture was stirred at 0° C. for 1 hour. After completion, the mixture was concentrated under reduced pressure. The residue was dissolved in DMF (5 mL) and CsF (284 mg, 1.87 mmol) was added. The mixture was stirred at room temperature for 16 hours. After completion, the mixture was diluted with water (30 mL) and extracted with EtOAc (2×30 mL). The organic layers were combined, dried over anhydrous Na₂SO₄, filtered and concentrated under reduced pressure. The residue was purified by Pre-HPLC (Agela Durashell C18, 30 mm×250 mm, 10 um; A: 0.1% TFA in water, B: CH₃CN, Gradient: 15% B to 50% B in 33 min at a flow rate of 40 mL/min, 285 nm) to give Compound 78 (1.1 mg, 4.09 yield, TFA salt). MS: m/z 632 [M+H]⁺.

### Example 79

### 4-(4-(2-oxa-6-azabicyclo[5.1.0]octan-6-yl)-5-ethoxy-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 79”)

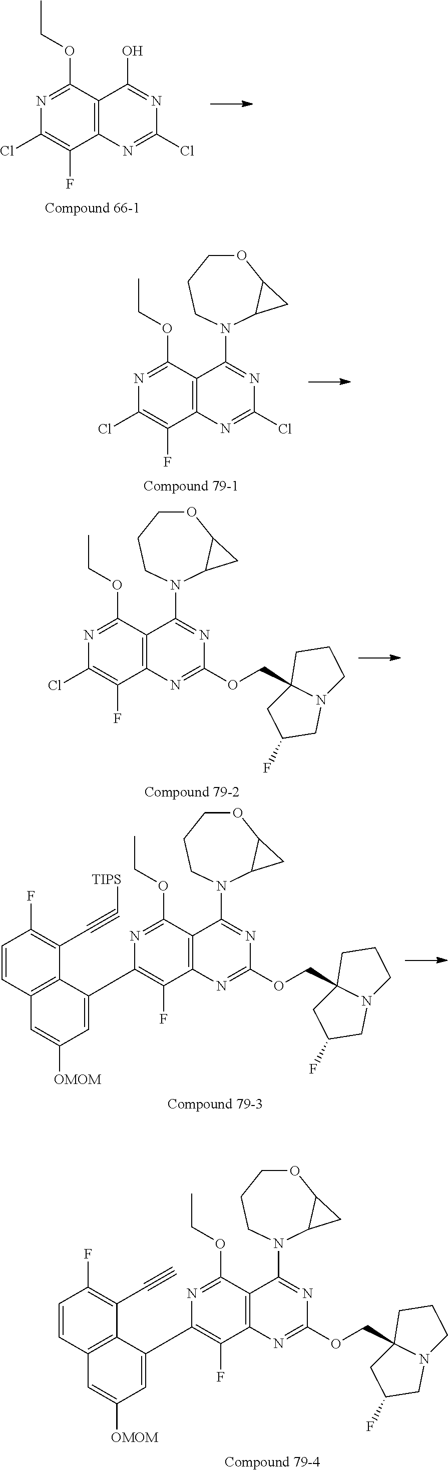

To a solution of Compound 66-1 (92 mg, 0.33 mmol) and DIEA (1 mL) in toluene (5 mL) was added phosphorus oxychloride (1 mL) then the mixture was stirred at 100° C. for 1.5 hours. Upon completion, the mixture was concentrated under reduced pressure. The residue was diluted with DCM (10 mL), then DIEA (2 mL) and Compound 7-5 (119 mg, 1.05 mmol) were added and the reaction mixture was stirred at room temperature for 1 hour. Another batch of Compound 7-5 (42 mg, 0.37 mmol) was added and the reaction mixture was stirred at room temperature for 16 hours. The mixture was diluted with water (30 mL), extracted with DCM (2×30 mL), the organic layers were combined, dried over anhydrous Na₂SO₄, filtered and concentrated under reduced pressure. The residue was purified by Prep-TLC to obtain Compound 79-1 (39 mg, 31.58% yield). MS: m/z 373 [M+H]⁺.

To a solution of Compound 79-1 (39 mg, 0.10 mmol) and INT 2 (41 mg, 0.26 mmol) in DMSO (5 mL) was added KF (32 mg, 0.55 mmol), then the mixture was stirred at 90° C. for 16 hours. After being cooled to room temperature, the mixture was diluted with water (30 mL) and extracted with EtOAc (2×30 mL). The organic layers were combined, dried over anhydrous Na₂SO₄, filtered and concentrated under reduced pressure. The residue was purified by Pre-TLC to obtain Compound 79-2 (69 mg, 133.14% yield, crude). MS: m/z 496 [M+H]⁺.

To a solution of Compound 79-2 (69 mg, 0.14 mmol), INT 3 (97 mg, 0.19 mmol) and Cs₂CO₃ (144 mg, 0.44 mmol) in toluene (4 mL) and water (1 mL) was added cataCXium A Pd G₃ (11 mg, 0.015 mmol). The mixture was purged with nitrogen followed by stirring at 100° C. for 4 hours. The reaction mixture was diluted with water (30 mL) and extracted with EtOAc (2×30 mL). The organic layers were combined, dried over anhydrous Na₂SO₄, filtered and concentrated under reduced pressure. The residue was purified by Pre-TLC (eluted with DCM:MeOH=10:1, v/v) to obtain Compound 79-3 (21 mg, 17.84% yield). MS: m/z 846 [M+1]⁺.

To a solution of Compound 79-3 (30 mg, 0.036 mmol) in acetonitrile (5 mL) was added HCl (4 M in 1,4-dioxane, 2 mL). The reaction mixture was stirred at 0° C. for 2 hours. After completion, the mixture was concentrated under reduced pressure. The residue was dissolved in DMF (5 mL) and CsF (766 mg, 5.04 mmol) was added. The mixture was stirred at room temperature for 16 hours. After completion, the mixture was diluted with water (20 mL) and extracted with EtOAc (2×20 mL). The organic layers were combined, dried over anhydrous Na₂SO₄, filtered and concentrated under reduced pressure. The residue was purified by Pre-HPLC (Agela Durashell C18, 30 mm×250 mm, 10 um; A: 0.1% TFA in water, B: CH₃CN, Gradient: 20% B to 59% B in 39 min at a flow rate of 40 mL/min, 240 nm) to give Compound 79 (5.5 mg, 34.32 yield, TFA salt). MS: m/z 646 [M+H]⁺.

The following compounds in the Table 21 were synthesized using the above procedures or modification procedures:

TABLE 21

| Example | Compound | Structure | IUNPAC Name |
|---|---|---|---|
| 80 | Compound 80 | 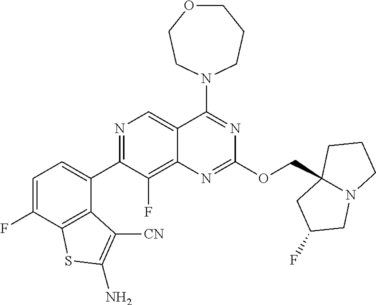 [SMILES: N#Cc1c(N)sc2c(F)ccc(-c3ncc4c(N5CCCOCC5)nc(OC[C@@]56CCCN5C[C@H](F)C6)nc4c3F)c12] | 2-amino-7-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)benzo[b]thiophene- 3-carbonitrile |
| 81 | Compound 81 | 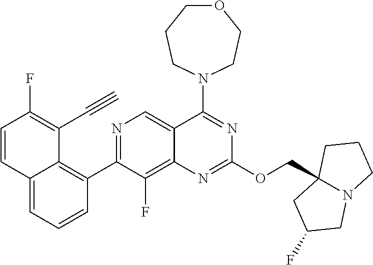 [SMILES: C#Cc1c(F)ccc2cccc(-c3ncc4c(N5CCCOCC5)nc(OC[C@@]56CCCN5C[C@H](F)C6)nc4c3F)c12] | 4-(7-(8-ethynyl-7-fluoronaphthalen-1-yl)- 8-fluoro-2-(((2R,7aS)-2-fluorotetrahydro- 1H-pyrrolizin-7a(5H)-yl)methoxy)pyrido [4,3-d]pyrimidin-4-yl)-1,4-oxazepane |
| 82 | Compound 82 | 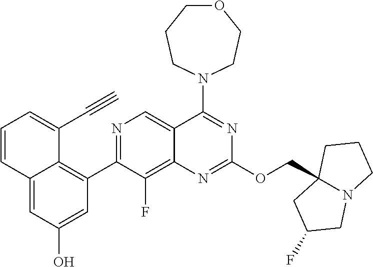 [SMILES: C#Cc1cccc2cc(O)cc(-c3ncc4c(N5CCCOCC5)nc(OC[C@@]56CCCN5C[C@H](F)C6)nc4c3F)c12] | 5-ethynyl-4-(8-fluoro-2-(((2R,7aS)-2-fluoro- tetrahydro-1H-pyrrolizin-7a(5H)-yl)methoxy)- 4-(1,4-oxazepan-4-yl)pyrido[4,3-d] pyrimidin-7-yl)naphthalen-2-ol |
| 83 | Compound 83 | 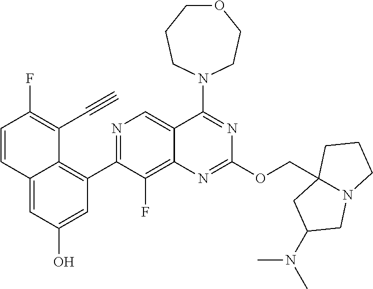 [SMILES: C#Cc1c(F)ccc2cc(O)cc(-c3ncc4c(N5CCCOCC5)nc(OCC56CCCN5CC(N(C)C)C6)nc4c3F)c12] | 4-(2-((2-(dimethylamino)tetrahydro-1H- pyrrolizin-7a(5H)-yl)methoxy)-8-fluoro-4- (1,4-oxazepan-4-yl)pyrido[4,3-d]pyrimidin- 7-yl)-5-ethynyl-6-fluoronaphthalen-2-ol |
| 84 | Compound 84 | 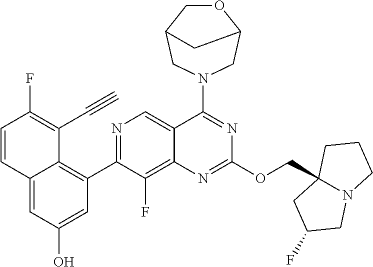 [SMILES: C#Cc1c(F)ccc2cc(O)cc(-c3ncc4c(N5CC6COC(C6)C5)nc(OC[C@@]56CCCN5C[C@H](F)C6)nc4c3F)c12] | 4-(4-(6-oxa-3-azabicyclo[3.2.1]octan-3-yl)- 8-fluoro-2-(((2R,7aS)-2-fluorotetrahydro- 1H-pyrrolizin-7a(5H)-yl)methoxy)pyrido [4,3-d]pyrimidin-7-yl)-5-ethynyl-6-fluoro naphthalen-2-ol |
| | Compound 84A | 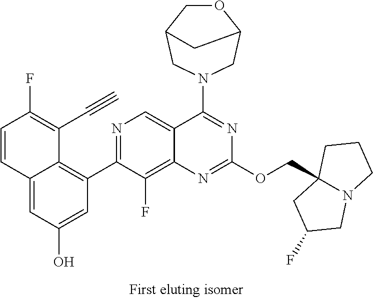 [SMILES: C#Cc1c(F)ccc2cc(O)cc(-c3ncc4c(N5CC6COC(C6)C5)nc(OC[C@@]56CCCN5C[C@H](F)C6)nc4c3F)c12] | 4-(4-((1R,5S)-6-oxa-3-azabicyclo[3.2.1]octan- 3-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol or 4-(4-((1S,5R)-6-oxa-3-azabicyclo[3.2.1]octan- 3-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol |
| | Compound 84B | 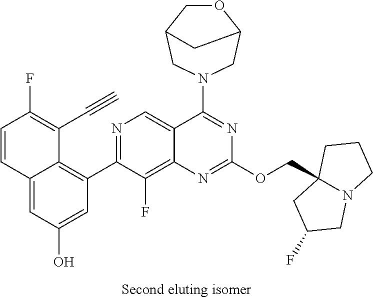 [SMILES: C#Cc1c(F)ccc2cc(O)cc(-c3ncc4c(N5CC6COC(C6)C5)nc(OC[C@@]56CCCN5C[C@H](F)C6)nc4c3F)c12] | 4-(4-((1S,5R)-6-oxa-3-azabicyclo[3.2.1]octan- 3-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol or 4-(4-((1R,5S)-6-oxa-3-azabicyclo[3.2.1]octan- 3-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol |
| 85 | Compound 85 | 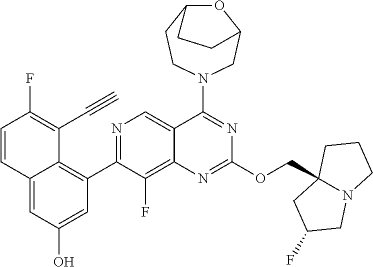 [SMILES: C#Cc1c(F)ccc2cc(O)cc(-c3ncc4c(N5CCC6CCC(C5)O6)nc(OC[C@@]56CCCN5C[C@H](F)C6)nc4c3F)c12] | 4-(4-(9-oxa-3-azabicyclo[4.2.1]nonan-3-yl)- 8-fluoro-2-(((2R,7aS)-2-fluorotetrahydro- 1H-pyrrolizin-7a(5H)-yl)methoxy)pyrido [4,3-d]pyrimidin-7-yl)-5-ethynyl-6-fluoro- naphthalen-2-ol |
| 86 | Compound 86 | 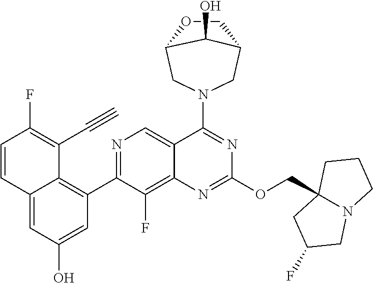 [SMILES: C#Cc1c(F)ccc2cc(O)cc(-c3ncc4c(N5C[C@H]6CO[C@@H](C5)[C@H]6O)nc(OC[C@@]56CCCN5C[C@H](F)C6)nc4c3F)c12] | (1S,5S,8S)-3-(7-(8-ethynyl-7-fluoro-3-hydroxy- naphthalen-1-yl)-8-fluoro-2-(((2R,7aS)- 2-fluorotetrahydro-1H-pyrrolizin-7a(5H)- yl)methoxy)pyrido[4,3-d]pyrimidin-4- yl)-6-oxa-3-azabicyclo[3.2.1]octan-8-ol |
| 87 | Compound 87 | 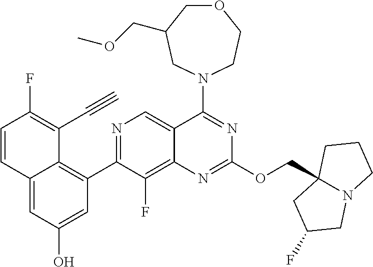 [SMILES: C#Cc1c(F)ccc2cc(O)cc(-c3ncc4c(N5CCOCC(COC)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(6-(methoxymethyl)-1,4- oxazepan-4-yl)pyrido[4,3-d]pyrimidin-7- yl)naphthalen-2-ol |
| 88 | Compound 88 | 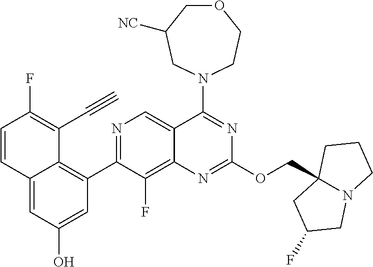 [SMILES: C#Cc1c(F)ccc2cc(O)cc(-c3ncc4c(N5CCOCC(C#N)C5)nc(OC[C@@]56CCCN5C[C@H](F)C6)nc4c3F)c12] | 4-(7-(8-ethynyl-7-fluoro-3-hydroxynaph- thalen-1-yl)-8-fluoro-2-(((2R,7aS)-2-fluoro tetrahydro-1H-pyrrolizin-7a(5H)-yl)methoxy) pyrido[4,3-d]pyrimidin-4-yl)-1,4-oxa zepane-6-carbonitrile |
| 89 | Compound 89 | 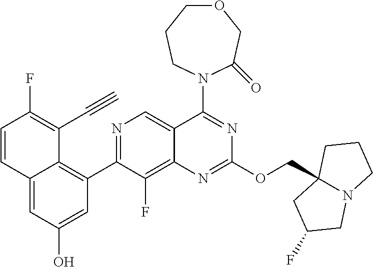 [SMILES: C#Cc1c(F)ccc2cc(O)cc(-c3ncc4c(N5CCCOCC5=O)nc(OC[C@@]56CCCN5C[C@H](F)C6)nc4c3F)c12] | 4-(7-(8-ethynyl-7-fluoro-3-hydroxynaph- thalen-1-yl)-8-fluoro-2-(((2R,7aS)-2-fluoro tetrahydro-1H-pyrrolizin-7a(5H)-yl)meth oxy)pyrido[4,3-d]pyrimidin-4-yl)-1,4-oxa zepan-3-one |
| 90 | Compound 90 | 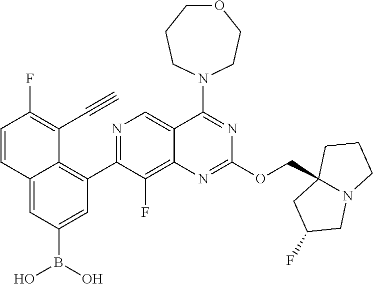 [SMILES: C#Cc1c(F)ccc2cc(B(O)O)cc(-c3ncc4c(N5CCCOCC5)nc(OC[C@@]56CCCN5C[C@H](F)C6)nc4c3F)c12] | (5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl) boronic acid |
| 91 | Compound 91 | 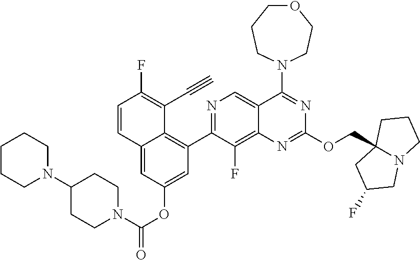 [SMILES: C#Cc1c(F)ccc2cc(OC(=O)N3CCC(N4CCCCC4)CC3)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl [1,4′-bipiperidine]-1′-carboxylate |
| 93 | Compound 93 | 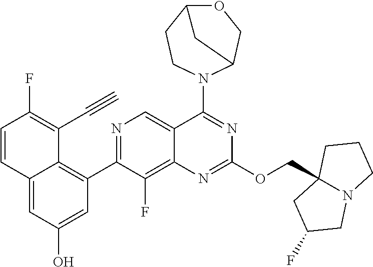 [SMILES: C#Cc1c(F)ccc2cc(O)cc(-c3ncc4c(N5CCC6CC5CO6)nc(OC[C@@]56CCCN5C[C@H](F)C6)nc4c3F)c12] | 4-(4-(6-oxa-2-azabicyclo[3.2.1]octan-2-yl)- 8-fluoro-2-(((2R,7aS)-2-fluorotetrahydro- 1H-pyrrolizin-7a(5H)-yl)methoxy)pyrido [4,3-d]pyrimidin-7-yl)-5-ethynyl-6-fluoro naphthalen-2-ol |
| | Compound 93A | 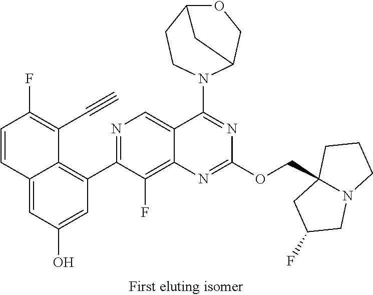 [SMILES: C#Cc1c(F)ccc2cc(O)cc(-c3ncc4c(N5CCC6CC5CO6)nc(OC[C@@]56CCCN5C[C@H](F)C6)nc4c3F)c12] | 4-(4-((1R,5S)-6-oxa-2-azabicyclo[3.2.1]octan- 2-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol or 4-(4-((1S,5R)-6-oxa-2-azabicyclo[3.2.1]octan- 2-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol |
| | Compound 93B | 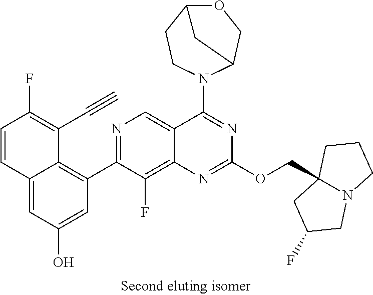 [SMILES: C#Cc1c(F)ccc2cc(O)cc(-c3ncc4c(N5CCC6CC5CO6)nc(OC[C@@]56CCCN5C[C@H](F)C6)nc4c3F)c12] | 4-(4-((1S,5R)-6-oxa-2-azabicyclo[3.2.1]octan- 2-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol or 4-(4-((1R,5S)-6-oxa-2-azabicyclo[3.2.1]octan- 2-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl- 6-fluoronaphthalen-2-ol |
| 94 | Compound 94 | 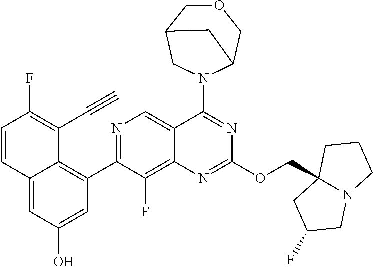 [SMILES: C#Cc1c(F)ccc2cc(O)cc(-c3ncc4c(N5CC6COCC5C6)nc(OC[C@@]56CCCN5C[C@H](F)C6)nc4c3F)c12] | 4-(4-(3-oxa-6-azabicyclo[3.2.1]octan-6-yl)- 8-fluoro-2-(((2R,7aS)-2-fluorotetrahydro- 1H-pyrrolizin-7a(5H)-yl)methoxy)pyrido [4,3-d]pyrimidin-7-yl)-5-ethynyl-6-fluoro naphthalen-2-ol |
| | Compound 94A | 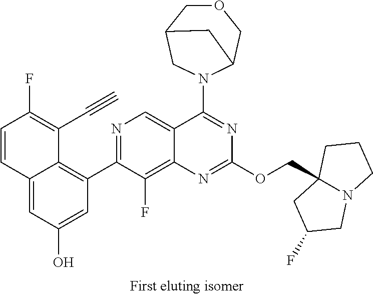 [SMILES: C#Cc1c(F)ccc2cc(O)cc(-c3ncc4c(N5CC6COCC5C6)nc(OC[C@@]56CCCN5C[C@H](F)C6)nc4c3F)c12] | 4-(4-((1R,5R)-3-oxa-6-azabicyclo[3.2.1] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-5-ethynyl- 6-fluoronaphthalen-2-ol or 4-(4-((1S,5S)-3-oxa-6-azabicyclo[3.2.1] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-5-ethynyl- 6-fluoronaphthalen-2-ol |
| | Compound 94B | 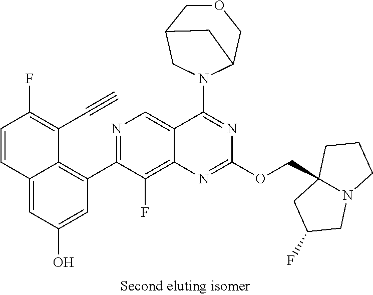 [SMILES: C#Cc1c(F)ccc2cc(O)cc(-c3ncc4c(N5CC6COCC5C6)nc(OC[C@@]56CCCN5C[C@H](F)C6)nc4c3F)c12] | 4-(4-((1S,5S)-3-oxa-6-azabicyclo[3.2.1] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-5-ethynyl- 6-fluoronaphthalen-2-ol or 4-(4-((1R,5R)-3-oxa-6-azabicyclo[3.2.1] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-5-ethynyl- 6-fluoronaphthalen-2-ol |
| 95 | Compound 95 | 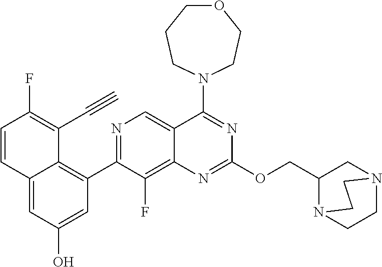 [SMILES: C#Cc1c(F)ccc2cc(O)cc(-c3ncc4c(N5CCCOCC5)nc(OCC5CN6CCN5CC6)nc4c3F)c12] | 4-(2-((1,4-diazabicyclo[2.2.2]octan-2-yl) methoxy)-8-fluoro-4-(1,4-oxazepan-4-yl) pyrido[4,3-d]pyrimidin-7-yl)-5-ethynyl-6- fluoronaphthalen-2-ol |
| 96 | Compound 96 | 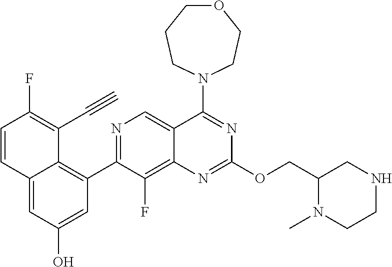 [SMILES: C#Cc1c(F)ccc2cc(O)cc(-c3ncc4c(N5CCCOCC5)nc(OCC5CNCCN5C)nc4c3F)c12] | 5-ethynyl-6-fluoro-4-(8-fluoro-2-((1-methyl- piperazin-2-yl)methoxy)-4-(1,4-oxazepan- 4-yl)pyrido[4,3-d]pyrimidin-7-yl)naph- thalen-2-ol |
| 97 | Compound 97 | 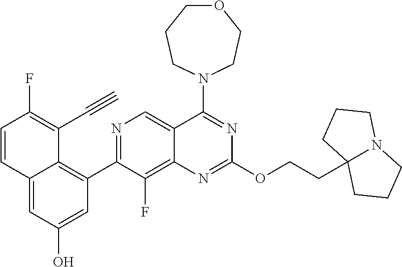 [SMILES: C#Cc1c(F)ccc2cc(O)cc(-c3ncc4c(N5CCCOCC5)nc(OCCC56CCCN5CCC6)nc4c3F)c12] | 5-ethynyl-6-fluoro-4-(8-fluoro-4-(1,4-oxa- zepan-4-yl)-2-(2-(tetrahydro-1H-pyrrolizin- 7a(5H)-yl)ethoxy)pyrido[4,3-d]pyrimidin- 7-yl)naphthalen-2-ol |
| 98 | Compound 98 | 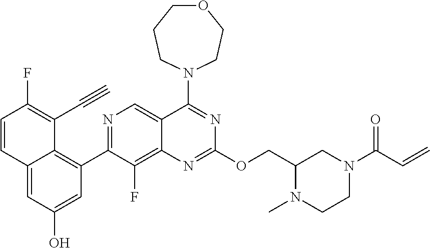 [SMILES: C#Cc1c(F)ccc2cc(O)cc(-c3ncc4c(N5CCCOCC5)nc(OCC5CN(C(=O)C=C)CCN5C)nc4c3F)c12] | 1-(3-(((7-(8-ethynyl-7-fluoro-3-hydroxy- naphthalen-1-yl)-8-fluoro-4-(1,4-oxazepan-4- yl)pyrido[4,3-d]pyrimidin-2-yl)oxy)methyl)- 4-methylpiperazin-1-yl)prop-2-en-1-one |
| 99 | Compound 99 | 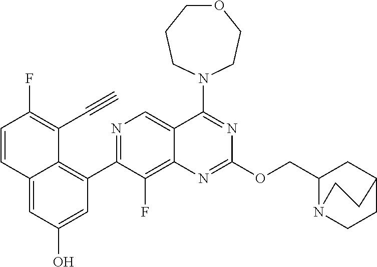 [SMILES: C#Cc1c(F)ccc2cc(O)cc(-c3ncc4c(N5CCCOCC5)nc(OCC5CC6CCN5CC6)nc4c3F)c12] | 5-ethynyl-6-fluoro-4-(8-fluoro-4-(1,4-oxa- zepan-4-yl)-2-(quinuclidin-2-ylmethoxy) pyrido[4,3-d]pyrimidin-7-yl)naphthalen-2-ol |
| 100 | Compound 100 | 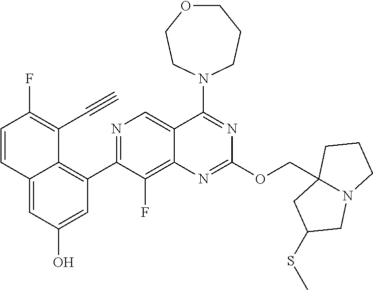 [SMILES: C#Cc1c(F)ccc2cc(O)cc(-c3ncc4c(N5CCCOCC5)nc(OCC56CCCN5CC(SC)C6)nc4c3F)c12] | 5-ethynyl-6-fluoro-4-(8-fluoro-2-((2-(methyl- thio)tetrahydro-1H-pyrrolizin-7a(5H)- yl)methoxy)-4-(1,4-oxazepan-4-yl)pyrido [4,3-d]pyrimidin-7-yl)naphthalen-2-ol |
| 101 | Compound 101 | 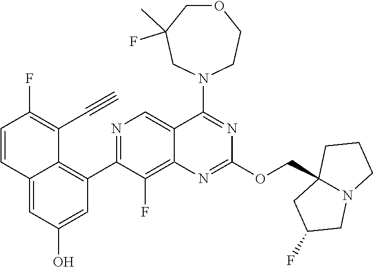 [SMILES: C#Cc1c(F)ccc2cc(O)cc(-c3ncc4c(N5CCOCC(C)(F)C5)nc(OC[C@@]56CCCN5C[C@H](F)C6)nc4c3F)c12] | 5-ethynyl-6-fluoro-4-(8-fluoro-4-(6-fluoro- 6-methyl-1,4-oxazepan-4-yl)-2-(((2R,7aS)- 2-fluorotetrahydro-1H-pyrrolizin-7a(5H)- yl)methoxy)pyrido[4,3-d]pyrimidin-7- yl)naphthalen-2-ol |
| 102 | Compound 102 | 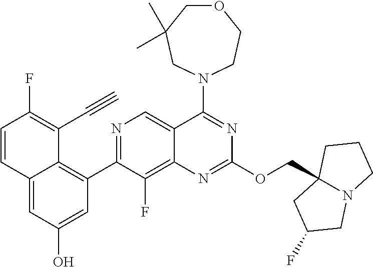 [SMILES: C#Cc1c(F)ccc2cc(O)cc(-c3ncc4c(N5CCOCC(C)(C)C5)nc(OC[C@@]56CCCN5C[C@H](F)C6)nc4c3F)c12] | 4-(4-(6,6-dimethyl-1,4-oxazepan-4-yl)-8- fluoro-2-(((2R,7aS)-2-fluorotetrahydro-1H- pyrrolizin-7a(5H)-yl)methoxy)pyrido[4,3- d]pyrimidin-7-yl)-5-ethynyl-6-fluoro- naphthalen-2-ol |
| 103 | Compound 103 | 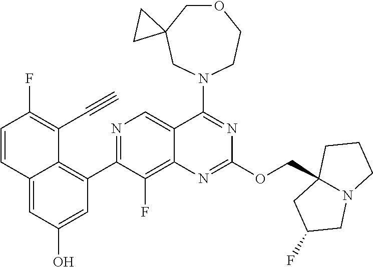 [SMILES: C#Cc1c(F)ccc2cc(O)cc(-c3ncc4c(N5CCOCC6(CC6)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(5-oxa-8-azaspiro[2.6] nonan-8-yl)pyrido[4,3-d]pyrimidin-7-yl) naphthalen-2-ol |
| 104 | Compound 104 | 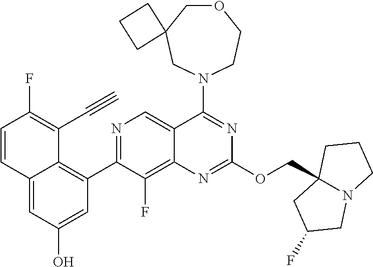 [SMILES: C#Cc1c(F)ccc2cc(O)cc(-c3ncc4c(N5CCOCC6(CCC6)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(6-oxa-9-azaspiro[3.6] decan-9-yl)pyrido[4,3-d]pyrimidin-7-yl) naphthalen-2-ol |
| 105 | Compound 105 | 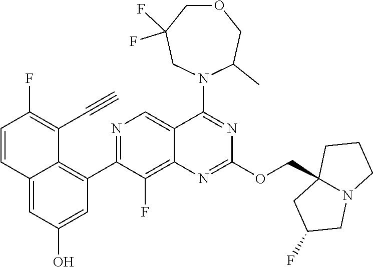 [SMILES: C#Cc1c(F)ccc2cc(O)cc(-c3ncc4c(N5CC(F)(F)COCC5C)nc(OC[C@@]56CCCN5C[C@H](F)C6)nc4c3F)c12] | 4-(4-(6,6-difluoro-3-methyl-1,4-oxazepan- 4-yl)-8-fluoro-2-(((2R,7aS)-2-fluorotetra- hydro-1H-pyrrolizin-7a(5H)-yl)methoxy) pyrido[4,3-d]pyrimidin-7-yl)-5-ethynyl-6- fluoronaphthalen-2-ol |
| 106 | Compound 106 | 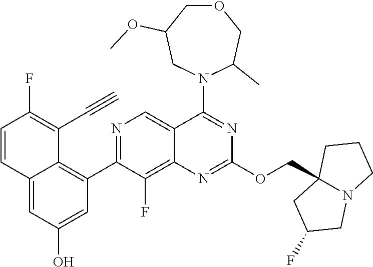 [SMILES: C#Cc1c(F)ccc2cc(O)cc(-c3ncc4c(N5CC(OC)COCC5C)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(6-methoxy-3-methyl-1,4- oxazepan-4-yl)pyrido[4,3-d]pyrimidin- 7-yl)naphthalen-2-ol |
| 107 | Compound 107 | 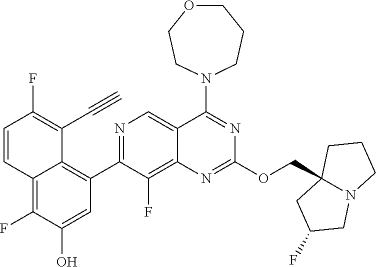 [SMILES: C#Cc1c(F)ccc2c(F)c(O)cc(-c3ncc4c(N5CCCOCC5)nc(OC[C@@]56CCCN5C[C@H](F)C6)nc4c3F)c12] | 5-ethynyl-1,6-difluoro-4-(8-fluoro-2-(((2R,7aS)- 2-fluorotetrahydro-1H-pyrrolizin-7a(5H)- yl)methoxy)-4-(1,4-oxazepan-4-yl) pyrido[4,3-d]pyrimidin-7-yl)naphthalen- 2-ol |
| 108 | Compound 108 | 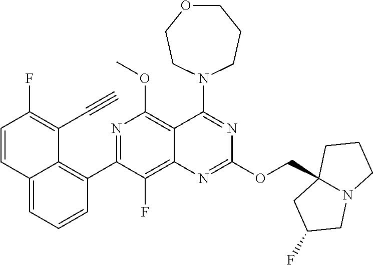 [SMILES: C#Cc1c(F)ccc2cccc(-c3nc(OC)c4c(N5CCCOCC5)nc(OC[C@@]56CCCN5C[C@H](F)C6)nc4c3F)c12] | 4-(7-(8-ethynyl-7-fluoronaphthalen-1-yl)- 8-fluoro-2-(((2R,7aS)-2-fluorotetrahydro- 1H-pyrrolizin-7a(5H)-yl)methoxy)-5- methoxypyrido[4,3-d]pyrimidin-4-yl)- 1,4-oxazepane |
| 109 | Compound 109 | 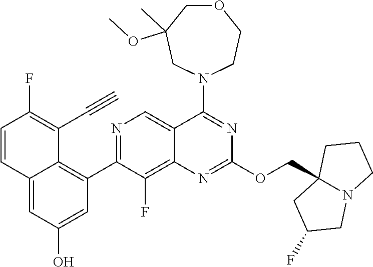 [SMILES: C#Cc1c(F)ccc2cc(O)cc(-c3ncc4c(N5CCOCC(C)(OC)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(6-methoxy-6-methyl-1,4- oxazepan-4-yl)pyrido[4,3-d]pyrimidin- 7-yl)naphthalen-2-ol |
| | Compound 109A | 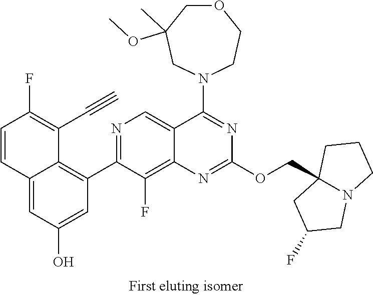 [SMILES: C#Cc1c(F)ccc2cc(O)cc(-c3ncc4c(N5CCOCC(C)(OC)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((S)-6-methoxy-6-methyl- 1,4-oxazepan-4-yl)pyrido[4,3-d]pyrimidin- 7-yl)naphthalen-2-ol or 5-ethynyl-6-fluoro-4-(8-fluoro-2-(((2R,7aS)- 2-fluorotetrahydro-1H-pyrrolizin-7a(5H)- yl)methoxy)-4-((R)-6-methoxy-6-methyl- 1,4-oxazepan-4-yl)pyrido[4,3-d]pyrimidin- 7-yl)naphthalen-2-ol |
| | Compound 109B | 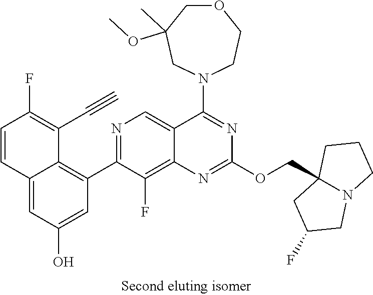 [SMILES: C#Cc1c(F)ccc2cc(O)cc(-c3ncc4c(N5CCOCC(C)(OC)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((R)-6-methoxy-6-methyl- 1,4-oxazepan-4-yl)pyrido[4,3-d]pyrimidin- 7-yl)naphthalen-2-ol or 5-ethynyl-6-fluoro-4-(8-fluoro-2-(((2R,7aS)- 2-fluorotetrahydro-1H-pyrrolizin-7a(5H)- yl)methoxy)-4-((S)-6-methoxy-6-methyl- 1,4-oxazepan-4-yl)pyrido[4,3-d]pyrimidin- 7-yl)naphthalen-2-ol |
| 110 | Compound 110 | 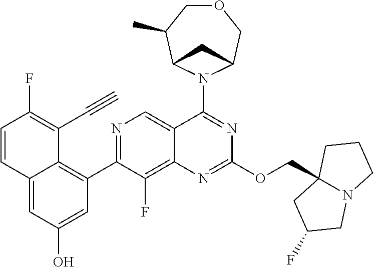 [SMILES: C#Cc1c(F)ccc2cc(O)cc(-c3ncc4c(N5[C@H]6COC[C@H](C)[C@@H]5C6)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((1R,5R,6S)-5-methyl-3- oxa-7-azabicyclo[4.1.1]octan-7-yl)pyrido [4,3-d]pyrimidin-7-yl)naphthalen-2-ol |
| 111 | Compound 111 | 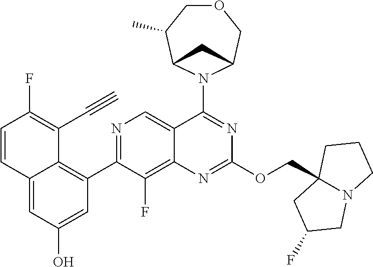 [SMILES: C#Cc1c(F)ccc2cc(O)cc(-c3ncc4c(N5[C@H]6COC[C@@H](C)[C@@H]5C6)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((1R,5S,6S)-5-methyl-3- oxa-7-azabicyclo[4.1.1]octan-7-yl)pyrido [4,3-d]pyrimidin-7-yl)naphthalen-2-ol |
| 112 | Compound 112 | 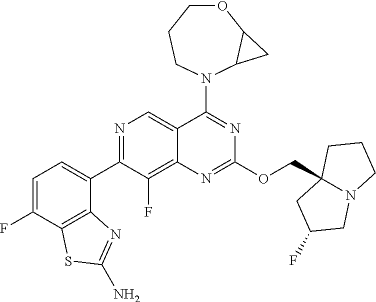 [SMILES: Nc1nc2c(-c3ncc4c(N5CCCOC6CC65)nc(OC[C@@]56CCCN5C[C@H](F)C6)nc4c3F)ccc(F)c2s1] | 4-(4-(2-oxa-6-azabicyclo[5.1.0]octan-6-yl)- 8-fluoro-2-(((2R,7aS)-2-fluorotetrahydro- 1H-pyrrolizin-7a(5H)-yl)methoxy)pyrido [4,3-d]pyrimidin-7-yl)-7-fluorobenzo[d] thiazol-2-amine |
| | Compound 112A | 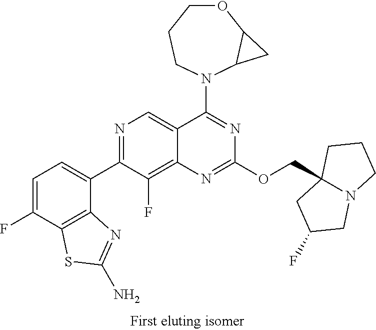 [SMILES: Nc1nc2c(-c3ncc4c(N5CCCOC6CC65)nc(OC[C@@]56CCCN5C[C@H](F)C6)nc4c3F)ccc(F)c2s1] | 4-(4-((1S,7R)-2-oxa-6-azabicyclo[5.1.0]octan- 6-yl)-8-fluoro-2-(((2R,7aS)-2-fluoro- tetrahydro-1H-pyrrolizin-7a(5H)-yl)methoxy) pyrido[4,3-d]pyrimidin-7-yl)-7-fluoro- benzo[d]thiazol-2-amine or 4-(4-((1R,7S)-2-oxa-6-azabicyclo[5.1.0] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-7-fluoro- benzo[d]thiazol-2-amine |
| | Compound 112B | 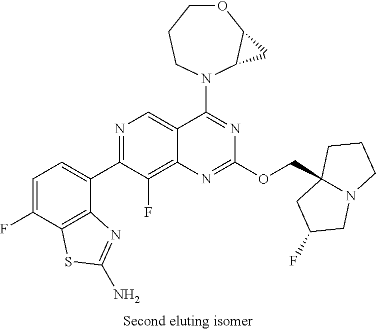 [SMILES: Nc1nc2c(-c3ncc4c(N5CCCO[C@@H]6C[C@@H]65)nc(OC[C@@]56CCCN5C[C@H](F)C6)nc4c3F)ccc(F)c2s1] | 4-(4-((1R,7S)-2-oxa-6-azabicyclo[5.1.0] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-7-fluoro- benzo[d]thiazol-2-amine or 4-(4-((1S,7R)-2-oxa-6-azabicyclo[5.1.0] octan-6-yl)-8-fluoro-2-(((2R,7aS)-2-fluoro- tetrahydro-1H-pyrrolizin-7a(5H)-yl)methoxy) pyrido[4,3-d]pyrimidin-7-yl)-7-fluoro- benzo[d]thiazol-2-amine |
| 113 | Compound 113 | 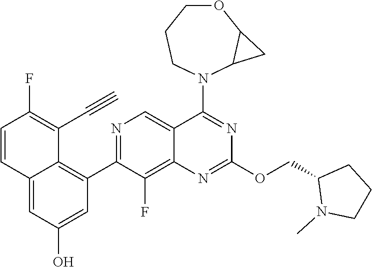 [SMILES: C#Cc1c(F)ccc2cc(O)cc(-c3ncc4c(N5CCCOC6CC65)nc(OC[C@@H]5CCCN5C)nc4c3F)c12] | 4-(4-(2-oxa-6-azabicyclo[5.1.0]octan-6-yl)- 8-fluoro-2-(((S)-1-methylpyrrolidin-2-yl) methoxy)pyrido[4,3-d]pyrimidin-7-yl)-5- ethynyl-6-fluoronaphthalen-2-ol |
| | Compound 113A | 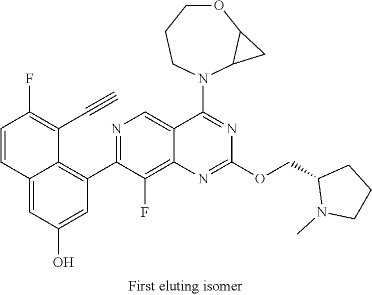 [SMILES: C#Cc1c(F)ccc2cc(O)cc(-c3ncc4c(N5CCCOC6CC65)nc(OC[C@@H]5CCCN5C)nc4c3F)c12] | 4-(4-((1S,7R)-2-oxa-6-azabicyclo[5.1.0] octan-6-yl)-8-fluoro-2-(((S)-1-methyl- pyrrolidin-2-yl)methoxy)pyrido[4,3-d] pyrimidin-7-yl)-5-ethynyl-6- fluoronaphthalen-2-ol or 4-(4-((1R,7S)-2-oxa-6-azabicyclo[5.1.0] octan-6-yl)-8-fluoro-2-(((S)-1-methyl- pyrrolidin-2-yl)methoxy)pyrido[4,3-d] pyrimidin-7-yl)-5-ethynyl-6- fluoronaphthalen-2-ol |
| | Compound 113B | 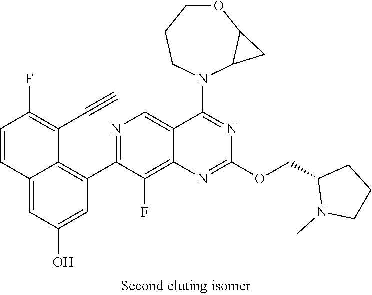 [SMILES: C#Cc1c(F)ccc2cc(O)cc(-c3ncc4c(N5CCCOC6CC65)nc(OC[C@@H]5CCCN5C)nc4c3F)c12] | 4-(4-((1R,7S)-2-oxa-6-azabicyclo[5.1.0] octan-6-yl)-8-fluoro-2-(((S)-1-methyl- pyrrolidin-2-yl)methoxy)pyrido[4,3-d] pyrimidin-7-yl)-5-ethynyl-6- fluoronaphthalen-2-ol or 4-(4-((1S,7R)-2-oxa-6-azabicyclo[5.1.0] octan-6-yl)-8-fluoro-2-(((S)-1-methyl- pyrrolidin-2-yl)methoxy)pyrido[4,3-d] pyrimidin-7-yl)-5-ethynyl-6- fluoronaphthalen-2-ol |
| 114 | Compound 114 | 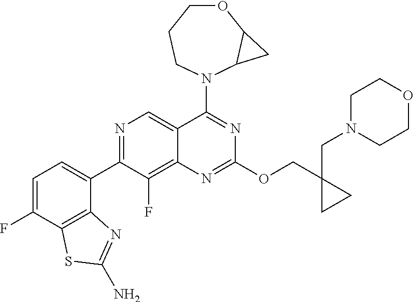 [SMILES: Nc1nc2c(-c3ncc4c(N5CCCOC6CC65)nc(OCC5(CN6CCOCC6)CC5)nc4c3F)ccc(F)c2s1] | 4-(4-(2-oxa-6-azabicyclo[5.1.0]octan-6-yl)- 8-fluoro-2-((1-(morpholinomethyl)cyclo- propyl)methoxy)pyrido[4,3-d]pyrimidin-7- yl)-7-fluorobenzo[d]thiazol-2-amine |
| 115 | Compound 115 | 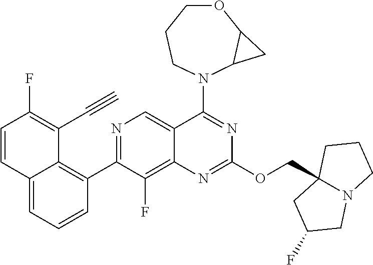 [SMILES: C#Cc1c(F)ccc2cccc(-c3ncc4c(N5CCCOC6CC65)nc(OC[C@@]56CCCN5C[C@H](F)C6)nc4c3F)c12] | 6-(7-(8-ethynyl-7-fluoronaphthalen-1-yl)- 8-fluoro-2-(((2R,7aS)-2-fluorotetrahydro- 1H-pyrrolizin-7a(5H)-yl)methoxy)pyrido [4,3-d]pyrimidin-4-yl)-2-oxa-6-azabicyclo [5.1.0]octane |
| 116 | Compound 116 | 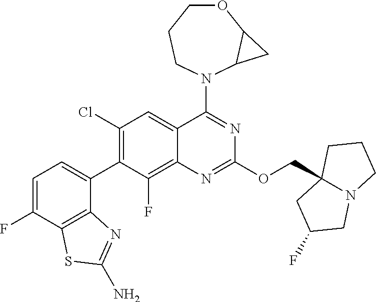 [SMILES: Nc1nc2c(-c3c(Cl)cc4c(N5CCCOC6CC65)nc(OC[C@@]56CCCN5C[C@H](F)C6)nc4c3F)ccc(F)c2s1] | 4-(4-(2-oxa-6-azabicyclo[5.1.0]octan-6-yl)- 6-chloro-8-fluoro-2-(((2R,7aS)-2-fluoro- tetrahydro-1H-pyrrolizin-7a(5H)-yl)methoxy) quinazolin-7-yl)-7-fluorobenzo[d]thiazol- 2-amine |
| 117 | Compound 117 | 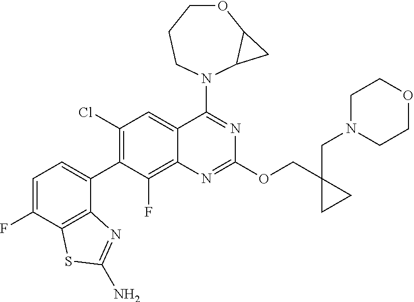 [SMILES: Nc1nc2c(-c3c(Cl)cc4c(N5CCCOC6CC65)nc(OCC5(CN6CCOCC6)CC5)nc4c3F)ccc(F)c2s1] | 4-(4-(2-oxa-6-azabicyclo[5.1.0]octan-6-yl)- 6-chloro-8-fluoro-2-((1-(morpholinomethyl) cyclopropyl)methoxy)quinazolin-7-yl)- 7-fluorobenzo[d]thiazol-2-amine |
| 118 | Compound 118 | 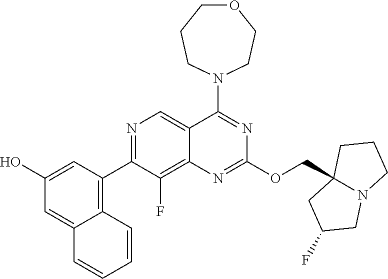 [SMILES: Oc1cc(-c2ncc3c(N4CCCOCC4)nc(OC[C@@]45CCCN4C[C@H](F)C5)nc3c2F)c2ccccc2c1] | 4-(8-fluoro-2-(((2R,7aS)-2-fluorotetrahydro- 1H-pyrrolizin-7a(5H)-yl)methoxy)-4- (1,4-oxazepan-4-yl)pyrido[4,3-d]pyrimidin- 7-yl)naphthalen-2-ol |
| 119 | Compound 119 | 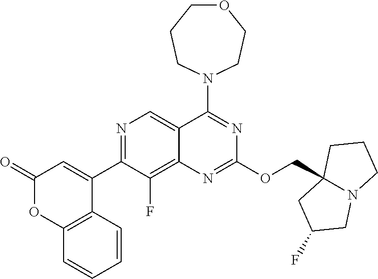 [SMILES: O=c1cc(-c2ncc3c(N4CCCOCC4)nc(OC[C@@]45CCCN4C[C@H](F)C5)nc3c2F)c2ccccc2o1] | 4-(8-fluoro-2-(((2R,7aS)-2-fluorotetrahydro- 1H-pyrrolizin-7a(5H)-yl)methoxy)-4- (1,4-oxazepan-4-yl)pyrido[4,3-d]pyrimidin- 7-yl)-2H-chromen-2-one |
| 120 | Compound 120 | 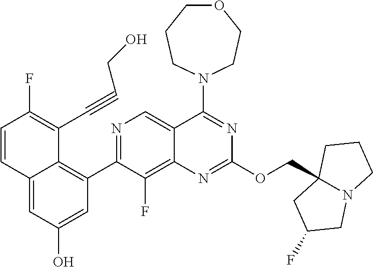 [SMILES: OCC#Cc1c(F)ccc2cc(O)cc(-c3ncc4c(N5CCCOCC5)nc(OC[C@@]56CCCN5C[C@H](F)C6)nc4c3F)c12] | 6-fluoro-4-(8-fluoro-2-(((2R,7aS)-2-fluoro tetrahydro-1H-pyrrolizin-7a(5H)-yl) methoxy)-4-(1,4-oxazepan-4-yl)pyrido [4,3-d]pyrimidin-7-yl)-5-(3-hydroxyprop- 1-yn-1-yl)naphthalen-2-ol |
| 121 | Compound 121 | 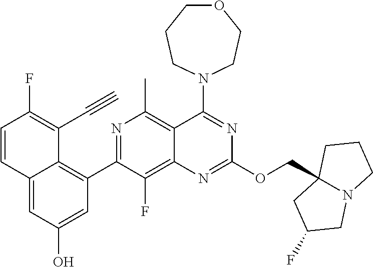 [SMILES: C#Cc1c(F)ccc2cc(O)cc(-c3nc(C)c4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-5-methyl-4-(1,4-oxazepan- 4-yl)pyrido[4,3-d]pyrimidin-7-yl) naphthalen-2-ol |
| 122 | Compound 122 | 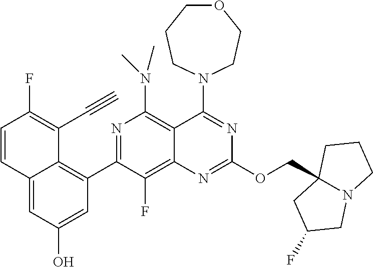 [SMILES: C#Cc1c(F)ccc2cc(O)cc(-c3nc(N(C)C)c4c(N5CCCOCC5)nc(OC[C@@]56CCCN5C[C@H](F)C6)nc4c3F)c12] | 4-(5-(dimethylamino)-8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)-5-ethynyl-6-fluoro- naphthalen-2-ol |
| 123 | Compound 123 | 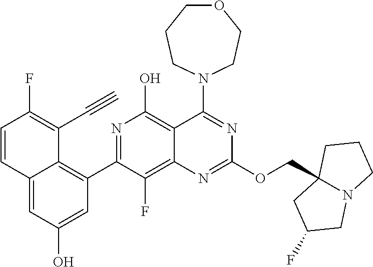 [SMILES: C#Cc1c(F)ccc2cc(O)cc(-c3nc(O)c4c(N5CCCOCC5)nc(OC[C@@]56CCCN5C[C@H](F)C6)nc4c3F)c12] | 7-(8-ethynyl-7-fluoro-3-hydroxynaphthalen- 1-yl)-8-fluoro-2-(((2R,7aS)-2-fluoro- tetrahydro-1H-pyrrolizin-7a(5H)-yl)methoxy)- 4-(1,4-oxazepan-4-yl)pyrido[4,3-d] pyrimidin-5-ol |
| 124 | Compound 124 | 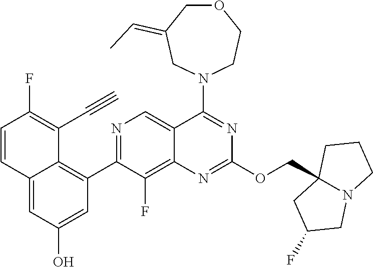 [SMILES: C#Cc1c(F)ccc2cc(O)cc(-c3ncc4c(N5CCOC/C(=C/C)C5)nc(OC[C@@]56CCCN5C[C@H](F)C6)nc4c3F)c12] | 4-(4-((E)-6-ethylidene-1,4-oxazepan-4-yl)- 8-fluoro-2-(((2R,7aS)-2-fluorotetrahydro- 1H-pyrrolizin-7a(5H)-yl)methoxy)pyrido [4,3-d]pyrimidin-7-yl)-5-ethynyl-6-fluoro- naphthalen-2-ol |
| 125 | Compound 125 | 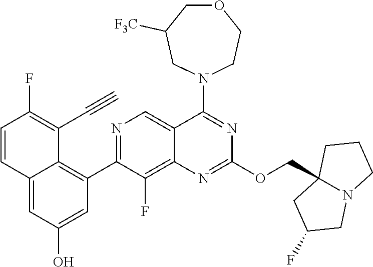 [SMILES: C#Cc1c(F)ccc2cc(O)cc(-c3ncc4c(N5CCOCC(C(F)(F)F)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(6-(trifluoromethyl)-1,4- oxazepan-4-yl)pyrido[4,3-d]pyrimidin-7- yl)naphthalen-2-ol |
| 126 | Compound 126 | 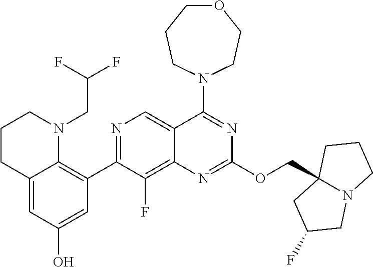 [SMILES: Oc1cc2c(c(-c3ncc4c(N5CCCOCC5)nc(OC[C@@]56CCCN5C[C@H](F)C6)nc4c3F)c1)N(CC(F)F)CCC2] | 1-(2,2-difluoroethyl)-8-(8-fluoro-2-(((2R,7aS)- 2-fluorotetrahydro-1H-pyrrolizin-7a(5H)- yl)methoxy)-4-(1,4-oxazepan-4-yl) pyrido[4,3-d]pyrimidin-7-yl)-1,2,3,4- tetrahydroquinolin-6-ol |
| 127 | Compound 127 | 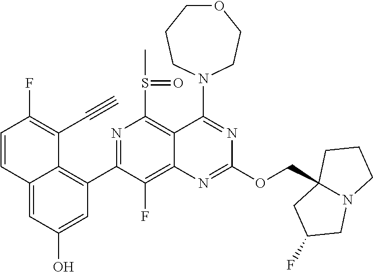 [SMILES: C#Cc1c(F)ccc2cc(O)cc(-c3nc(S(C)=O)c4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-5-(methylsulfinyl)-4-(1,4- oxazepan-4-yl)pyrido[4,3-d]pyrimidin-7- yl)naphthalen-2-ol |
| 128 | Compound 128 | 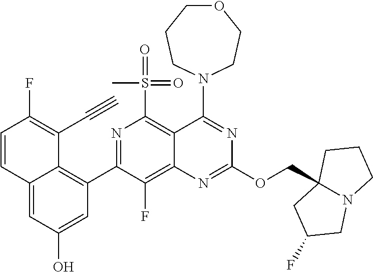 [SMILES: C#Cc1c(F)ccc2cc(O)cc(-c3nc(S(C)(=O)=O)c4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-5-(methylsulfonyl)-4-(1,4- oxazepan-4-yl)pyrido[4,3-d]pyrimidin-7- yl)naphthalen-2-ol |
| 129 | Compound 129 | 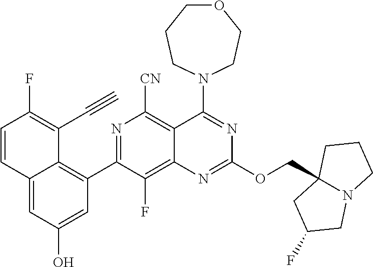 [SMILES: C#Cc1c(F)ccc2cc(O)cc(-c3nc(C#N)c4c(N5CCCOCC5)nc(OC[C@@]56CCCN5C[C@H](F)C6)nc4c3F)c12] | 7-(8-ethynyl-7-fluoro-3-hydroxynaphthalen- 1-yl)-8-fluoro-2-(((2R,7aS)-2-fluorotetra- hydro-1H-pyrrolizin-7a(5H)-yl)methoxy)- 4-(1,4-oxazepan-4-yl)pyrido[4,3-d] pyrimidine-5-carbonitrile |
| 130 | Compound 130 | 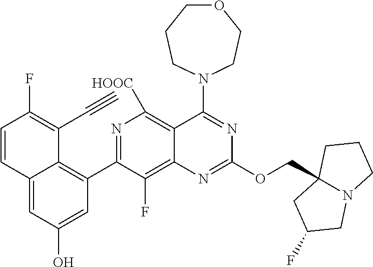 [SMILES: C#Cc1c(F)ccc2cc(O)cc(-c3nc(C(=O)O)c4c(N5CCCOCC5)nc(OC[C@@]56CCCN5C[C@H](F)C6)nc4c3F)c12] | 7-(8-ethynyl-7-fluoro-3-hydroxynaphthalen- 1-yl)-8-fluoro-2-(((2R,7aS)-2-fluorotetra- hydro-1H-pyrrolizin-7a(5H)-yl)methoxy)- 4-(1,4-oxazepan-4-yl)pyrido[4,3-d] pyrimidine-5-carboxylic acid |
| 131 | Compound 131 | 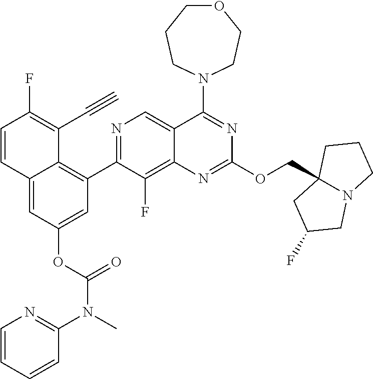 [SMILES: C#Cc1c(F)ccc2cc(OC(=O)N(C)c3ccccn3)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl methyl(pyridin-2-yl)carbamate |
| 132 | Compound 132 | 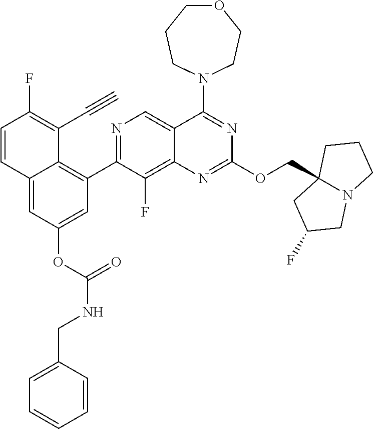 [SMILES: C#Cc1c(F)ccc2cc(OC(=O)NCc3ccccc3)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl benzylcarbamate |
| 133 | Compound 133 | 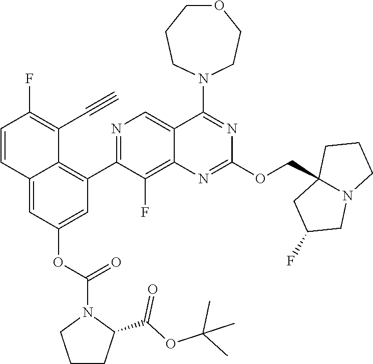 [SMILES: C#Cc1c(F)ccc2cc(OC(=O)N3CCC[C@H]3C(=O)OC(C)(C)C)cc(-c3ncc4c(N5CCCOCC5)nc(OC[C@@]56CCCN5C[C@H](F)C6)nc4c3F)c12] | 2-(tert-butyl) 1-(5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl) pyrido[4,3-d]pyrimidin-7-yl)naphthalen- 2-yl)(S)-pyrrolidine-1,2-dicarboxylate |
| 134 | Compound 134 | 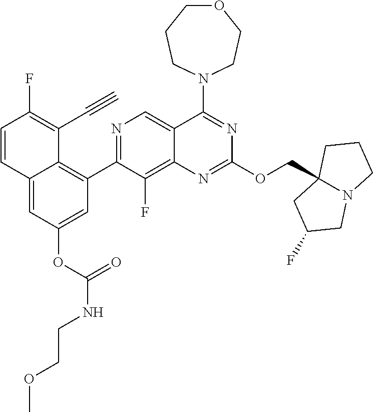 [SMILES: C#Cc1c(F)ccc2cc(OC(=O)NCCOC)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl (2-methoxyethyl)carbamate |
| 135 | Compound 134 | 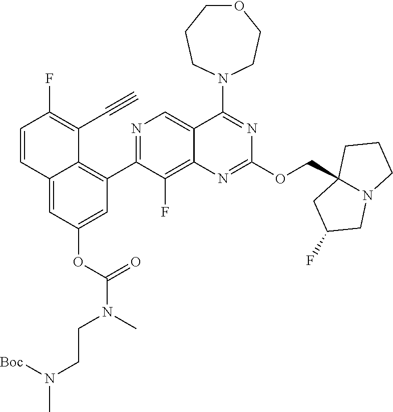 [SMILES: C#Cc1c(F)ccc2cc(OC(=O)N(C)CCN(C)C(=O)OC(C)(C)C)cc(-c3ncc4c(N5CCCOCC5)nc(OC[C@@]56CCCN5C[C@H](F)C6)nc4c3F)c12] | tert-butyl (5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl) ethane-1,2-diylbis(methylcarbamate) |
| 136 | Compound 136 | 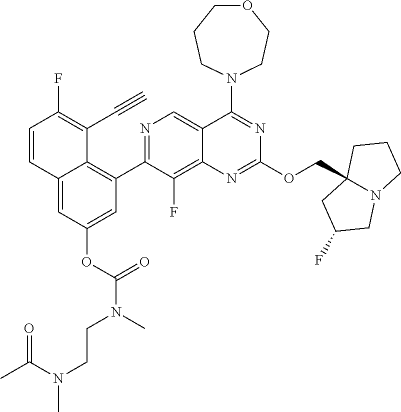 [SMILES: C#Cc1c(F)ccc2cc(OC(=O)N(C)CCN(C)C(C)=O)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl methyl(2-(N-methylacetamido)ethyl) carbamate |
| 137 | Compound 137 | 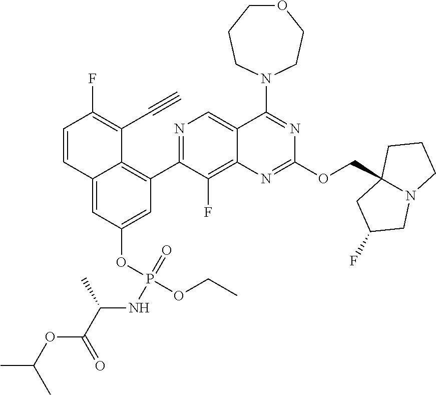 [SMILES: C#Cc1c(F)ccc2cc(OP(=O)(N[C@@H](C)C(=O)OC(C)C)OCC)cc(-c3ncc4c(N5CCCOCC5)nc(OC[C@@]56CCCN5C[C@H](F)C6)nc4c3F)c12] | isopropyl (ethoxy((5-ethynyl-6-fluoro-4-(8-fluoro-2- (((2R,7aS)-2-fluorotetrahydro-1H-pyrrolizin- 7a(5H)-yl)methoxy)-4-(1,4-oxazepan- 4-yl)pyrido[4,3-d]pyrimidin-7-yl)naphthalen- 2-yl)oxy)phosphoryl)-L-alaninate |
| 138 | Compound 138 | 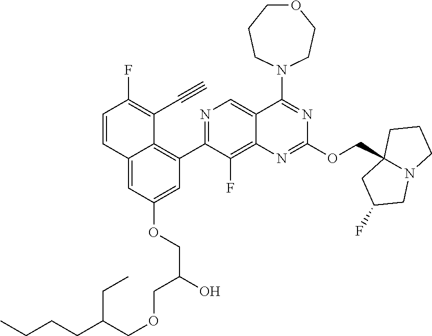 [SMILES: C#Cc1c(F)ccc2cc(OCC(O)COCC(CC)CCCC)cc(-c3ncc4c(N5CCCOCC5)nc(OC[C@@]56CCCN5C[C@H](F)C6)nc4c3F)c12] | 1-((2-ethylhexyl)oxy)-3-((5-ethynyl-6-fluoro- 4-(8-fluoro-2-(((2R,7aS)-2-fluorotetra- hydro-1H-pyrrolizin-7a(5H)-yl)methoxy)- 4-(1,4-oxazepan-4-yl)pyrido[4,3-d]pyrimidin- 7-yl)naphthalen-2-yl)oxy)propan-2-ol |
| 139 | Compound 139 | 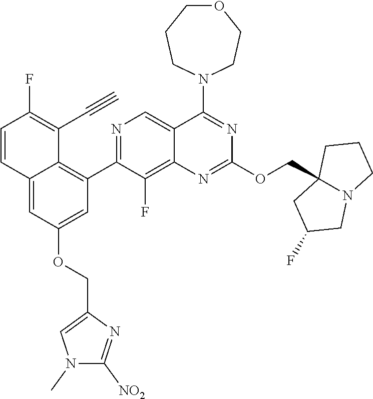 [SMILES: C#Cc1c(F)ccc2cc(OCc3cn(C)c([N+](=O)[O-])n3)cc(-c3ncc4c(N5CCCOCC5)nc(OC[C@@]56CCCN5C[C@H](F)C6)nc4c3F)c12] | 4-(7-(8-ethynyl-7-fluoro-3-((1-methyl-2- nitro-1H-imidazol-4-yl)methoxy)naphthalen- 1-yl)-8-fluoro-2-(((2R,7aS)-2-fluorotetra- hydro-1H-pyrrolizin-7a(5H)-yl)methoxy) pyrido[4,3-d]pyrimidin-4-yl)-1,4-oxazepane |
| 140 | Compound 140 | 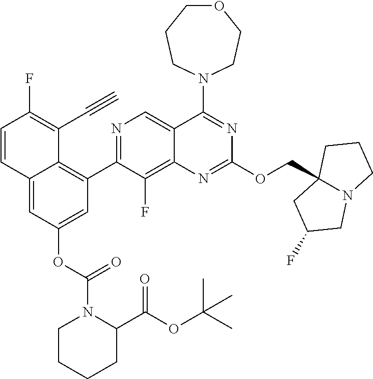 [SMILES: C#Cc1c(F)ccc2cc(OC(=O)N3CCCCC3C(=O)OC(C)(C)C)cc(-c3ncc4c(N5CCCOCC5)nc(OC[C@@]56CCCN5C[C@H](F)C6)nc4c3F)c12] | 2-(tert-butyl) 1-(5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl) pyrido[4,3-d]pyrimidin-7-yl)naphthalen- 2-yl)piperidine-1,2-dicarboxylate |
| 141 | Compound 141 | 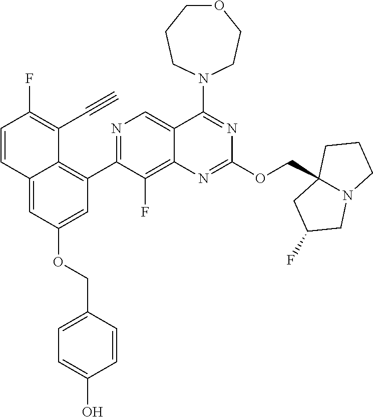 [SMILES: C#Cc1c(F)ccc2cc(OCc3ccc(O)cc3)cc(-c3ncc4c(N5CCCOCC5)nc(OC[C@@]56CCCN5C[C@H](F)C6)nc4c3F)c12] | 4-(((5-ethynyl-6-fluoro-4-(8-fluoro-2-(((2R,7aS)- 2-fluorotetrahydro-1H-pyrrolizin- 7a(5H)-yl)methoxy)-4-(1,4-oxazepan-4-yl) pyrido[4,3-d]pyrimidin-7-yl)naphthalen- 2-yl)oxy)methyl)phenol |
| 142 | Compound 142 | 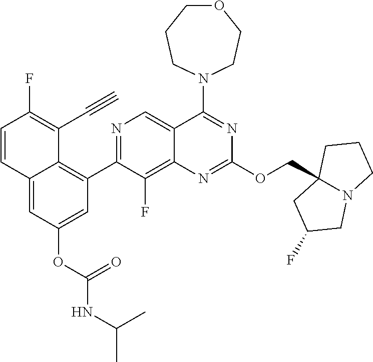 [SMILES: C#Cc1c(F)ccc2cc(OC(=O)NC(C)C)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl isopropylcarbamate |
| 143 | Compound 143 | 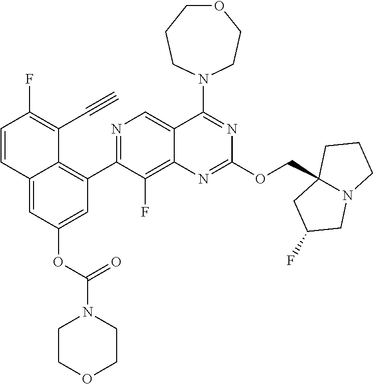 [SMILES: C#Cc1c(F)ccc2cc(OC(=O)N3CCOCC3)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl morpholine-4-carboxylate |
| 144 | Compound 144 | 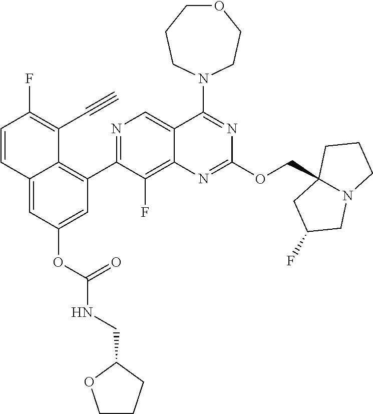 [SMILES: C#Cc1c(F)ccc2cc(OC(=O)NC[C@@H]3CCCO3)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl (((S)-tetrahydrofuran-2-yl)methyl) carbamate |
| 145 | Compound 145 | 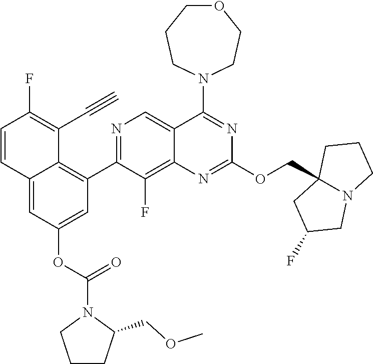 [SMILES: C#Cc1c(F)ccc2cc(OC(=O)N3CCC[C@H]3COC)cc(-c3ncc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl)pyrido [4,3-d]pyrimidin-7-yl)naphthalen-2-yl (S)-2-(methoxymethyl)pyrrolidine-1- carboxylate |
| 146 | Compound 146 | 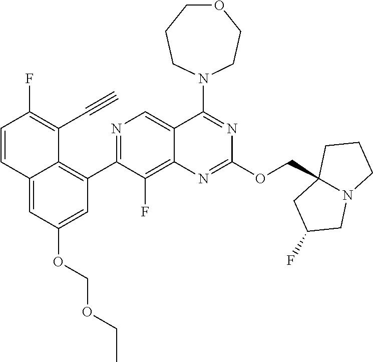 [SMILES: C#Cc1c(F)ccc2cc(OCOCC)cc(-c3ncc4c(N5CCCOCC5)nc(OC[C@@]56CCCN5C[C@H](F)C6)nc4c3F)c12] | 4-(7-(3-(ethoxymethoxy)-8-ethynyl-7-fluoro- naphthalen-1-yl)-8-fluoro-2-(((2R,7aS)- 2-fluorotetrahydro-1H-pyrrolizin-7a(5H)- yl)methoxy)pyrido[4,3-d]pyrimidin-4-yl)- 1,4-oxazepane |
| 147 | Compound 147 | 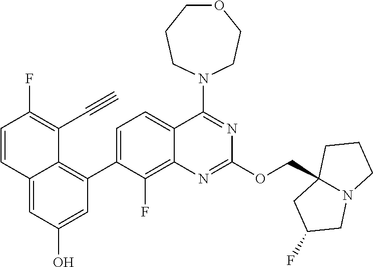 [SMILES: C#Cc1c(F)ccc2cc(O)cc(-c3ccc4c(N5CCCOCC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(1,4-oxazepan-4-yl) quinazolin-7-yl)naphthalen-2-ol |
| 148 | Compound 148 | 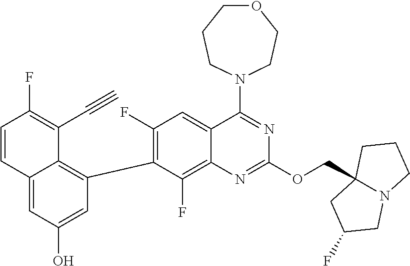 [SMILES: C#Cc1c(F)ccc2cc(O)cc(-c3c(F)cc4c(N5CCCOCC5)nc(OC[C@@]56CCCN5C[C@H](F)C6)nc4c3F)c12] | 4-(6,8-difluoro-2-(((2R,7aS)-2-fluorotetra- hydro-1H-pyrrolizin-7a(5H)-yl)methoxy)- 4-(1,4-oxazepan-4-yl)quinazolin-7-yl)-5- ethynyl-6-fluoronaphthalen-2-ol |
| | Compound 148A | 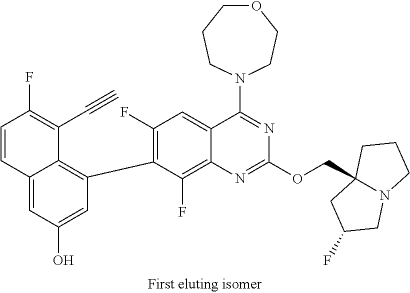 [SMILES: C#Cc1c(F)ccc2cc(O)cc(-c3c(F)cc4c(N5CCCOCC5)nc(OC[C@@]56CCCN5C[C@H](F)C6)nc4c3F)c12] | (P)-4-(6,8-difluoro-2-(((2R,7aS)-2-fluoro- tetrahydro-1H-pyrrolizin-7a(5H)-yl) methoxy)-4-(1,4-oxazepan-4-yl)quinazolin- 7-yl)-5-ethynyl-6-fluoronaphthalen-2-ol or (M)-4-(6,8-difluoro-2-(((2R,7aS)-2-fluoro- tetrahydro-1H-pyrrolizin-7a(5H)-yl) methoxy)-4-(1,4-oxazepan-4-yl) quinazolin-7-yl)-5-ethynyl-6- fluoronaphthalen-2-ol |
| | Compound 148B | 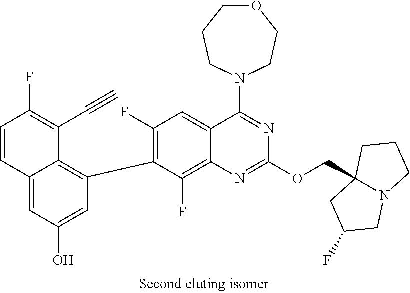 [SMILES: C#Cc1c(F)ccc2cc(O)cc(-c3c(F)cc4c(N5CCCOCC5)nc(OC[C@@]56CCCN5C[C@H](F)C6)nc4c3F)c12] | (M)-4-(6,8-difluoro-2-(((2R,7aS)-2-fluoro- tetrahydro-1H-pyrrolizin-7a(5H)-yl)methoxy)- 4-(1,4-oxazepan-4-yl)quinazolin-7-yl)- 5-ethynyl-6-fluoronaphthalen-2-ol or (P)-4-(6,8-difluoro-2-(((2R,7aS)-2-fluoro- tetrahydro-1H-pyrrolizin-7a(5H)-yl)methoxy)- 4-(1,4-oxazepan-4-yl)quinazolin-7-yl)- 5-ethynyl-6-fluoronaphthalen-2-ol |
| 149 | Compound 149 | 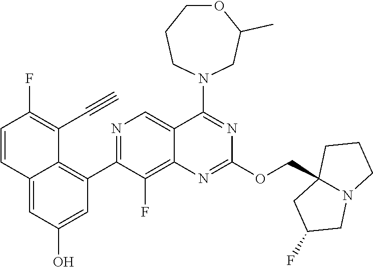 [SMILES: C#Cc1c(F)ccc2cc(O)cc(-c3ncc4c(N5CCCOC(C)C5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(2-methyl-1,4-oxazepan- 4-yl)pyrido[4,3-d]pyrimidin-7-yl) naphthalen-2-ol |
| 150 | Compound 150 | 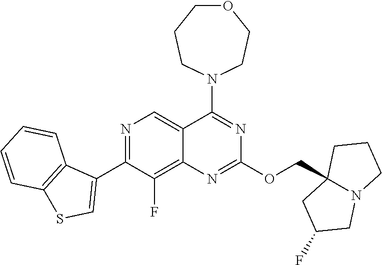 [SMILES: Fc1c(-c2csc3ccccc23)ncc2c(N3CCCOCC3)nc(OC[C@@]34CCCN3C[C@H](F)C4)nc12] | 4-(7-(benzo[b]thiophen-3-yl)-8-fluoro-2- (((2R,7aS)-2-fluorotetrahydro-1H-pyrrolizin- 7a(5H)-yl)methoxy)pyrido[4,3-d]pyrimidin- 4-yl)-1,4-oxazepane |
| 151 | Compound 151 | 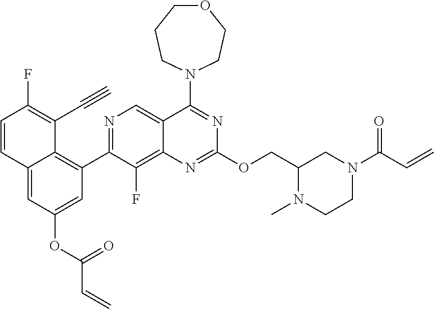 [SMILES: C#Cc1c(F)ccc2cc(OC(=O)C=C)cc(-c3ncc4c(N5CCCOCC5)nc(OCC5CN(C(=O)C=C)CCN5C)nc4c3F)c12] | 4-(2-((4-acryloyl-1-methylpiperazin-2-yl) methoxy)-8-fluoro-4-(1,4-oxazepan-4-yl) pyrido[4,3-d]pyrimidin-7-yl)-5-ethynyl-6- fluoronaphthalen-2-yl acrylate |
| 152 | Compound 152 | 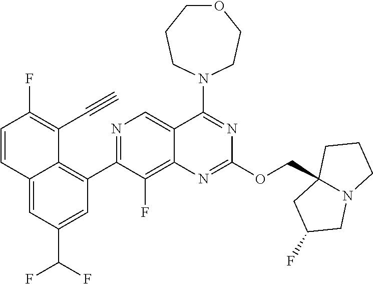 [SMILES: C#Cc1c(F)ccc2cc(C(F)F)cc(-c3ncc4c(N5CCCOCC5)nc(OC[C@@]56CCCN5C[C@H](F)C6)nc4c3F)c12] | 4-(7-(3-(difluoromethyl)-8-ethynyl-7-fluoro- naphthalen-1-yl)-8-fluoro-2-(((2R,7aS)- 2-fluorotetrahydro-1H-pyrrolizin-7a(5H)- yl)methoxy)pyrido[4,3-d]pyrimidin-4-yl)- 1,4-oxazepane |
| 153 | Compound 153 | 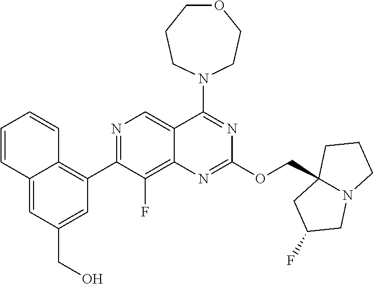 [SMILES: OCc1cc(-c2ncc3c(N4CCCOCC4)nc(OC[C@@]45CCCN4C[C@H](F)C5)nc3c2F)c2ccccc2c1] | (4-(8-fluoro-2-(((2R,7aS)-2-fluorotetra- hydro-1H-pyrrolizin-7a(5H)-yl)methoxy)-4- (1,4-oxazepan-4-yl)pyrido[4,3-d]pyrimidin- 7-yl)naphthalen-2-yl)methanol |
| 154 | Compound 154 | 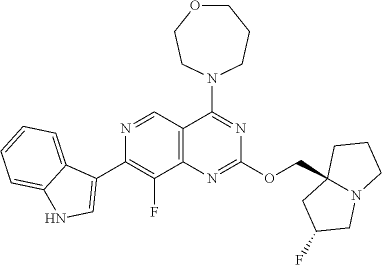 [SMILES: Fc1c(-c2c[nH]c3ccccc23)ncc2c(N3CCCOCC3)nc(OC[C@@]34CCCN3C[C@H](F)C4)nc12] | 4-(8-fluoro-2-(((2R,7aS)-2-fluorotetrahydro- 1H-pyrrolizin-7a(5H)-yl)methoxy)-7- (1H-indol-3-yl)pyrido[4,3-d]pyrimidin-4- yl)-1,4-oxazepane |

Note:
Compound 84 was separated by Prep-HPLC-Gilson with the following conditions: Column, CHIRAL ART Cellulose-SA column(2 cm × 25 cm, 5 μm); mobile phase, Hex(0.2% isopropylamine): EtOH = 70:30; Flowing rate: 20 ml/min. This results in Compound 84A (the first eluting isomer, Retention Time 8.038 min) and Compound 84B (the second eluting isomer, Retention Time 9.341 min).
Compound 93 was separated by Prep-HPLC-Gilson with the following conditions: Column, CHIRAL ART Cellulose-SB column (2 cm × 25 cm, 5 μm; mobile phase, (Hex:DCM = 3:1)(0.2% isopropylamine): EtOH = 50:50; Flowing rate: 20 ml/min. This results in Compound 93A (the first eluting isomer, Retention Time 4.456 min) and Compound 93B (the second eluting isomer, Retention Time 6.759 min).
Compound 94 was separated by Prep-HPLC-Gilson with the following conditions: Column, CHIRAL ART Cellulose-SB column (2 cm × 25 cm, 5 μm); mobile phase, (Hex:DCM = 3:1)(0.2% isopropylamine): EtOH = 50:50; Flowing rate: 20 ml/min. This results in Compound 94A (the first eluting isomer, Retention Time 4.355 min) and Compound 94B (the second eluting isomer, Retention Time 6.364 min).
Compound 109 was separated by Prep-HPLC-Gilson with the following conditions: Column, CHIRAL ART Cellulose-IG column (2 cm × 25 cm, 5 μm); mobile phase, (Hex:DCM = 3:1)(0.2% isopropylamine): EtOH = 80:20; Flowing rate: 20 ml/min. This results in Compound 109A (the first eluting isomer, Retention Time 4.995 min) and Compound 109B (the second eluting isomer, Retention Time 5.87 min).
Compound 148 was separated by Prep-HPLC-Gilson with the following conditions: Column, CHIRAL ART Cellulose-ID column (2 cm × 25 cm, 5 μm); mobile phase, Hex (0.2% isopropylamine): EtOH = 70:30; Flowing rate: 16 ml/min. This results in Compound 148A (the first eluting isomer, Retention Time 7.192 min) and Compound 148B (the second eluting isomer, Retention Time 8.042 min).

### Pharmacological Experiments

### 1. SOS1 Catalyzed Nucleotide Exchange Assay

The inhibition activity of each of compounds on GDP form K-Ras was evaluated by SOS1 catalyzed nucleotide exchange assays. K-Ras G12D, K-Ras G12V, K-Ras G12C, K-Ras G13D, K-Ras G12A, K-Ras G12R, K-Ras Q61H and K-Ras WT proteins were used in this assay.

Briefly, K-Ras (His tag, aa 1-169) pre-loaded with GDP was pre-incubated with each of compounds in the presence of 10 nM GDP in a 384-well plate (Greiner) for 15-60 mins, then purified SOS1 ExD (Flag tag, aa 564-1049), BODIPY™ FL GTP (Invitrogen) and monoclonal antibody anti 6HIS—Tb cryptate Gold (Cisbio) were added to the assay wells and incubated for 4 hours at 25° C. (Specially, we did not add SOS1 in the K-Ras G13D assay). Wells containing same percent of DMSO served as vehicle control, and wells without K-Ras served as low control. TR-FRET signals were read on Tecan Spark multimode microplate reader. The parameters were F486: Excitation 340 nm, Emission 486 nm, Lag time 100 μs, Integration time 200 s; F515: Excitation 340 nm, Emission 515 nm, Lag time 100 μs, Integration time 200 μs. TR-FRET ratios for each individual wells were calculated by equation: TR-FRET ratio=(Signal F515/Signal F486)*10000. The percent of activation of compounds treated wells were normalized between vehicle control and low control (% Activation=(TR-FRET ratio_{Compound treated}−TR-FRET ratio_{Low control})/(TR-FRET ratio_{Vehicle control}−TR-FRET ratio_{Low control})*100). Then the data were analyzed either by fitting a 4-parameter logistic model or by Excel to calculate IC₅₀ values. The results are showed in the following Table 22.

2. GTP-K-Ras and cRAF Interaction Assay

The inhibition activity of each of compounds on GTP form K-Ras was evaluated by GppNp-K-Ras and cRAF interaction assays. GppNp is an analog of GTP. K-Ras G12D, K-Ras G12V, K-Ras G12C, K-Ras G13D, K-Ras G12A, K-Ras G12R, K-Ras Q61H and K-Ras WT proteins were used in this assay.

Briefly, K-Ras (His tag, aa 1-169) pre-loaded with GppNp was pre-incubated with each of compounds in the presence of 200 μM GTP in a 384-well plate (Greiner) for 15-60 mins, then cRAF RBD (GST tag, aa 50-132, CreativeBioMart), monoclonal antibody anti GST-d2 (Cisbio) and monoclonal antibody anti 6HIS—Tb cryptate Gold (Cisbio) were added to the assay wells and incubated for 2 hours at 25° C. Wells containing same percent of DMSO served as vehicle control, and wells without K-Ras served as low control. HTRF signals were read on Tecan Spark multimode microplate reader and HTRF ratios were calculated under manufacturer's instructions. The percent of activation of compounds treated wells were normalized between vehicle control and low control (% Activation=(HTRF ratio_{Compound treated}−HTRF ratio_{Low control})/(HTRF ratio_{Vehicle control}−HTRF ratio_{Low control})*100%). Then the data were analyzed either by fitting a 4-parameter logistic model or by Excel to calculate IC₅₀ values. The results are showed in the following Table 22.

TABLE 22

| | Biochemical Activity (IC₅₀, nM) | | | | | | | |
|---|---|---|---|---|---|---|---|---|
| | K-Ras G12D | | K-Ras G12V | | K-Ras G12C | | K-Ras G13D | |
| Compound | GDP | GppNp | GDP | GppNp | GDP | GppNp | GDP | GppNp |
| Compound 1 | 0.924 | 6.00 | 1.21 | 11.4 | 0.274 | 12.9 | 0.339 | 0.975 |
| Compound 2 | 3.66 | 152 | 4.99 | 207 | | | | |
| Compound 3 | 0.724 | | 1.31 | | | | | |
| Compound 4 | 127 | | 1002 | | | | | |
| Compound 5 | 2.93 | | 4.18 | | | | | |
| Compound 6 | 21.2 | | 25.0 | | | | | |
| Compound 7 | 0.665 | 2.42 | 1.23 | 4.74 | 1.20 | 43.6 | 1.35 | 2.13 |
| Compound 7A | 105 | 775 | 88.4 | 1762 | | | | |
| Compound 7B | 0.554 | 1.36 | 0.367 | 3.47 | 0.293 | 20.5 | 0.920 | 1.49 |
| Compound 8A | 3.05 | 151 | 2.49 | 170 | | | 8.64 | 21.4 |
| Compound 8B | 0.81 | 10.2 | 1.53 | 16.3 | | | 2.08 | 2.60 |
| Compound 9 | 2.67 | 302 | 4.67 | 576 | | | 10.0 | 34.6 |
| Compound 10 | 1.18 | 23.1 | 1.28 | 40.6 | | | 4.69 | 7.00 |
| Compound 11 | 0.724 | 10.5 | 1.42 | 16.2 | 0.855 | 69.4 | 1.35 | 2.18 |
| Compound 12A | 1.81 | 63.2 | 1.48 | 184 | | | 7.83 | 8.85 |
| Compound 12B | 0.748 | 8.67 | 0.972 | 18.2 | 0.345 | 58.9 | 1.36 | 2.33 |
| Compound 13A | 4.92 | 380 | 4.08 | 1207 | | | 18.3 | 59.2 |
| Compound 13B | 0.67 | 5.25 | 0.658 | 35.7 | | | 2.13 | 1.57 |
| Compound 14A | 6.25 | 210 | 9.61 | 1484 | | | 35.2 | 26.8 |
| Compound 14B | 0.739 | 7.31 | 0.515 | 158 | | | 2.76 | 1.87 |
| Compound 15A | 38.2 | 890 | 23.7 | 2069 | | | >200 | >100 |
| Compound 15B | 1.08 | 69.7 | 0.873 | 331 | | | 2.87 | 10.8 |
| Compound 16 | 1.65 | 21.2 | 1.47 | 35.7 | | | 4.96 | 3.54 |
| Compound 16A | 3.78 | 315 | 6.05 | 469 | | | 14.2 | 37.2 |
| Compound 16B | 0.576 | 7.46 | 0.890 | 10.4 | 1.12 | 58.3 | 2.22 | 2.30 |
| Compound 17 | 65.7 | 322 | 26.8 | 796 | | | 100 | 61.2 |
| Compound 18 | 1.11 | 31.0 | 1.41 | 107 | | | 1.42 | 2.30 |
| Compound 19 | 11.1 | 89.5 | 9.69 | 221 | | | 24.9 | 18.6 |
| Compound 19B | 5.62 | 57.0 | 5.27 | 136 | 3.69 | 302 | 14.4 | 7.88 |
| Compound 20 | 6.03 | 298 | 8.81 | 1634 | | | 7.93 | 18.2 |
| Compound 21 | 0.985 | 6.72 | 0.813 | 13.4 | | | 0.416 | 4.81 |
| Compound 22 | 3.43 | 506 | 3.20 | 1439 | | | 4.02 | 23.9 |
| Compound 23 | 1.99 | ~100 | 2.75 | 239 | | | >100 | >100 |
| Compound 24 | 1.28 | 114 | 0.708 | 181 | | | 1.91 | 6.28 |
| Compound 25 | 0.739 | 210 | 2.35 | 435 | 3.27 | >1000 | 2.51 | 10.6 |
| Compound 26 | 15.5 | 440 | 5.77 | 4150 | | | | |
| Compound 27 | 2.78 | 197 | 8.13 | 720 | | | | |
| Compound 28B | 1.06 | 4.83 | 2.31 | 29.2 | | | | |
| Compound 29 | 0.741 | 4.38 | 0.503 | 8.51 | 0.539 | 45.6 | | |
| Compound 30 | 1.37 | 18.6 | 1.91 | 39.8 | | | | |
| Compound 31 | 1.59 | 25.0 | 2.47 | 49.1 | | | | |
| Compound 32A | 1.13 | 27.3 | 1.37 | 62.6 | | | 5.13 | 9.88 |
| Compound 32B | 1.93 | 39.2 | 1.97 | 69.7 | | | 4.41 | 6.59 |
| Compound 33 | 2.13 | 26.9 | 3.74 | 54.8 | 1.20 | 136 | 1.31 | 2.79 |
| Compound 34 | 2.43 | 246 | 2.12 | 483 | | | | |
| Compound 35 | 0.984 | 7.18 | 5.81 | 22.1 | | | | |
| Compound 36 | 2.79 | 324 | 7.96 | 267 | | | | |
| Compound 37 | 6.09 | 75.5 | 6.71 | 147 | | | 100 | >100 |
| Compound 38 | 2.34 | 8.79 | 1.30 | 16.3 | 1.41 | 103 | 2.29 | 3.42 |
| Compound 39 | 0.992 | 4.75 | 1.10 | 7.04 | 1.14 | 68.3 | 1.95 | 2.29 |
| Compound 40 | 1.74 | 58 | 1.94 | 124 | | | 2.60 | 10.3 |
| Compound 41 | 2.92 | 12.7 | 6.88 | 32.4 | | | 70.7 | 1.30 |
| Compound 42 | 1.14 | 114 | 0.99 | 247 | 1.46 | 556 | 2.25 | 4.55 |
| Compound 43 | 4.30 | 219 | 3.11 | 375 | | | 9.26 | 18.7 |
| Compound 44 | 1.46 | | 0.862 | | | | | |
| Compound 45 | 2.37 | | 4.96 | | | | | |
| Compound 46A | 5.20 | 172 | 3.12 | 343 | | | 20.4 | 61.6 |
| Compound 46B | 0.96 | 9.12 | 1.26 | 50.9 | | | 1.83 | 3.39 |
| Compound 47 | 2.77 | 86.8 | 4.38 | 152 | | | | |
| Compound 48 | 5.86 | | 4.32 | | | | | |
| Compound 49A | 34.0 | 2172 | 11.0 | 5618 | | | 24.7 | >100 |
| Compound 49B | 2.16 | 115 | 0.770 | 552 | | | >100 | >100 |
| Compound 50 | 15.7 | 1995 | 7.99 | 4186 | | | 17.8 | >100 |
| Compound 51 | 1.12 | 10.1 | 1.69 | 22.0 | | | | |
| Compound 52 | 1.48 | 9.69 | 1.88 | 16.6 | | | | |
| Compound 53 | 1.16 | 18.3 | 1.66 | 50.5 | 1.11 | 137 | 1.36 | 4.73 |
| Compound 54 | 1.01 | | | | | | | |
| Compound 55 | 1.35 | | 2.12 | | | | | |
| Compound 56 | 4.69 | | 3.91 | | | | | |
| Compound 57 | 5.39 | | 9.28 | | | | | |
| Compound 59 | 0.866 | | 1.34 | | | | | |
| Compound 60 | 0.664 | | 1.32 | | | | | |
| Compound 61 | 0.653 | | 1.28 | | | | | |
| Compound 62 | 4.13 | | 6.51 | | | | | |
| Compound 63 | 1.22 | | 1.63 | | | | | |
| Compound 64 | 1.61 | 48.9 | 2.16 | 200 | 0.965 | 274 | 1.49 | 2.61 |
| Compound 65 | 0.749 | 2.77 | 1.42 | 6.11 | 0.452 | 37.0 | 1.93 | 2.38 |
| Compound 66 | 1.03 | 3.69 | 0.117 | 7.84 | 0.416 | 38.5 | 0.965 | 2.77 |
| Compound 67 | 5.50 | | 7.32 | | | | | |
| Compound 68 | 438 | | 1133 | | | | | |
| Compound 69 | 119 | | 202 | | | | | |
| Compound 70 | 30.0 | | 27.1 | | | | | |
| Compound 71 | 163 | | 180 | | | | | |
| Compound 73 | 20.7 | | 18.7 | | | | | |
| Compound 74 | 136 | | 69.1 | | | | | |
| Compound 75 | 38.4 | | 51.4 | | | | | |
| Compound 80 | 120 | 2763 | 53.4 | 7354 | | | >100 | >100 |
| Compound 81 | 30.7 | 2239 | 42.3 | 3931 | | | 132 | 213 |
| Compound 82 | 1.85 | 36.8 | 1.66 | 63.1 | | | 2.18 | 3.24 |
| Compound 83 | 4.91 | 235 | 3.11 | 343 | | | | |
| Compound 84 | 1.33 | 9.77 | 1.92 | 42.5 | | | 1.92 | 4.49 |
| Compound 85 | 9.92 | 144 | 4.44 | 369 | | | 11.9 | 24.9 |
| Compound 86 | 20.5 | 91.4 | 2.66 | 275 | | | | |
| Compound 87 | 1.33 | 12.5 | 2.25 | 374 | | | | |
| Compound 88 | 47.6 | 1255 | 13.6 | 2849 | | | 100 | >100 |
| Compound 89 | 218 | 3944 | 62.9 | 9803 | | | | |
| Compound 90 | 27.2 | | 26.5 | | | | | |
| Compound 84A | 7.76 | 1019 | 3.43 | 3723 | | | 7.44 | 56.7 |
| Compound 84B | 0.652 | 6.75 | 0.983 | 21.6 | 3.19 | 76.8 | 2.72 | 1.76 |
| Compound 93 | 157 | 1309 | 19.9 | 2880 | | | >200 | >100 |
| Compound 93 | 31.5 | 552 | 4.42 | 2031 | | | 22.7 | 21.1 |
| Compound 94 | 30.4 | 141 | 11.8 | 416 | | | >200 | 100 |
| Compound 96 | 27.8 | 1695 | 18.6 | 2771 | | | 114 | >100 |
| Compound 98 | 205 | 5975 | 54.5 | 8191 | | | >200 | >100 |
| Compound 99 | 22.4 | 1394 | 22.6 | 2911 | | | >100 | >100 |
| Compound 100 | 1.45 | 8.06 | 1.20 | 15.1 | 0.802 | 64.9 | 2.49 | 3.80 |
| Compound 101 | 1.19 | 125 | 2.70 | 298 | | | 2.18 | 100 |
| Compound 102 | 10.6 | 358 | 10.2 | 2108 | | | | 39.2 |
| Compound 103 | 14.6 | 377 | 10.9 | 1892 | | | | |
| Compound 104 | 83.6 | 2421 | 146 | >10000 | | | | |
| Compound 105 | 4.85 | 232 | 2.08 | 378 | | | | |
| Compound 106 | 3.40 | 159 | 3.95 | 831 | | | 5.84 | 6.08 |
| Compound 107 | 5.18 | 256 | 11.9 | 331 | | | 12.8 | 67.7 |
| Compound 108 | 30.1 | 666 | 23.7 | 1708 | | | | |
| Compound 109 | 8.44 | 916 | 4.95 | 2225 | | | 29.3 | >100 |
| Compound 109 | 251 | 8234 | 39.0 | >10000 | | | 100 | >100 |
| Compound 110 | 14.5 | 336 | 6.63 | >1000 | | | | |
| Compound 111 | 4.40 | 192 | 4.21 | >1000 | | | | |
| Compound 113 | 0.970 | 6.15 | 1.66 | 29.0 | | | | |
| Compound 115 | 7.04 | 298 | 7.80 | ~1000 | | | 22.8 | 53.2 |
| Compound 118 | 14.3 | 1754 | 12.3 | 2601 | | | 100 | >100 |
| Compound 120 | 29.6 | 1297 | 12.9 | 4125 | | | 100 | >100 |
| Compound 121 | 1.44 | 90.9 | 3.01 | 140 | | | | |
| Compound 123 | 3.61 | 74.0 | 2.49 | 156 | | | | |
| Compound 124 | 6.15 | 390 | 2.57 | 805 | | | | |
| Compound 125 | 3.22 | 27.2 | 3.35 | 286 | | | | |
| Compound 126 | 76.7 | >1000 | 84.6 | >1000 | | | | |
| Compound 127 | 93.1 | >1000 | 88.5 | >1000 | | | | |
| Compound 129 | 9.63 | >1000 | 19.8 | >1000 | | | | |
| Compound 139 | 112 | | 92.7 | | | | | |
| Compound 147 | 1.64 | 31.2 | 1.68 | 70.0 | | | 1.16 | 1.68 |
| Compound 148 | 3.61 | 13.9 | 1.66 | 22.8 | 0.709 | 58.5 | 1.57 | 1.45 |
| Compound 149 | 2.08 | 16.0 | 2.71 | 23.5 | | | 4.75 | 5.59 |

| | Biochemical Activity (IC₅₀, nM) | | | | | | | |
|---|---|---|---|---|---|---|---|---|
| | K-Ras WT | | K-Ras G12A | | K-Ras G12R | | K-Ras Q61H | |
| Compound | GDP | GppNp | GDP | GppNp | GDP | GppNp | GDP | GppNp |
| Compound 1 | 0.675 | 18.6 | 3.25 | 62.8 | 0.570 | 3.53 | 0.683 | 11.1 |
| Compound 2 | | | | | | | | |
| Compound 3 | 0.741 | | | | | | | |
| Compound 4 | 479 | | | | | | | |
| Compound 5 | 2.71 | | | | | | | |
| Compound 6 | 39.7 | | | | | | | |
| Compound 7 | 0.342 | 9.87 | 0.674 | 23.0 | 1.04 | 3.14 | 1.04 | 8.91 |
| Compound 7A | 143 | | | | | | | |
| Compound 7B | 0.587 | 4.21 | 0.353 | 6.90 | 0.438 | 1.71 | 0.619 | 2.59 |
| Compound 8A | 4.70 | 255 | | | | | | |
| Compound 8B | 1.71 | 36.4 | | | | | | |
| Compound 9 | 11.5 | 505 | | | | | | |
| Compound 10 | 2.05 | 62.1 | | | | | | |
| Compound 11 | 1.03 | 21.0 | 1.34 | 86.8 | 0.750 | 22.2 | 0.893 | 48.3 |
| Compound 12A | 2.26 | 105 | | | | | | |
| Compound 12B | 0.66 | 24.1 | 0.387 | 66.4 | 0.472 | 4.83 | 0.706 | 15.9 |
| Compound 13A | 5.99 | 794 | | | | | | |
| Compound 13B | 0.76 | 14.2 | | | | | | |
| Compound 14A | 12.9 | 755 | | | | | | |
| Compound 14B | 0.882 | 15.8 | | | | | | |
| Compound 15A | 60.2 | | | | | | | |
| Compound 15B | 1.40 | 173 | | | | | | |
| Compound 16 | | | | | | | | |
| Compound 16A | 3.88 | 741 | | | | | | |
| Compound 16B | 1.72 | 21.5 | 1.64 | 98.7 | 0.994 | 15.7 | 1.15 | 38.5 |
| Compound 17 | | | | | | | | |
| Compound 18 | | | | | | | | |
| Compound 19 | 16.30 | | | | | | | |
| Compound 19B | 8.72 | 183 | 15.7 | 493 | 3.17 | 95.9 | 6.65 | 212 |
| Compound 20 | 12.7 | | | | | | | |
| Compound 21 | 0.790 | | | | | | | |
| Compound 22 | 4.05 | | | | | | | |
| Compound 23 | 3.18 | 134 | | | | | | |
| Compound 24 | 1.33 | 79.9 | | | | | | |
| Compound 25 | 2.70 | 781 | 3.96 | >1000 | 2.70 | 574 | 3.37 | 964 |
| Compound 26 | 4.72 | | | | | | | |
| Compound 27 | 9.51 | 235 | | | | | | |
| Compound 28B | | | | | | | | |
| Compound 29 | 0.777 | 29.5 | | | | | | |
| Compound 30 | 1.99 | | | | | | | |
| Compound 31 | 2.61 | | | | | | | |
| Compound 32A | 2.66 | 70.4 | | | | | | |
| Compound 32B | 3.12 | 77.4 | | | | | | |
| Compound 33 | 0.934 | 58.7 | 0.685 | 142 | 0.938 | 37.9 | 1.01 | 53.0 |
| Compound 34 | | | | | | | | |
| Compound 35 | | | | | | | | |
| Compound 36 | | | | | | | | |
| Compound 37 | 9.93 | 228 | | | | | | |
| Compound 38 | 1.52 | 29.0 | 1.57 | 86.7 | 1.03 | 48.5 | 1.21 | 60.9 |
| Compound 39 | 1.14 | 12.0 | 0.996 | 151 | 0.825 | 12.5 | 1.24 | 32.9 |
| Compound 40 | | | | | | | | |
| Compound 41 | 14.9 | 82.4 | | | | | | |
| Compound 42 | 1.26 | 171 | 1.65 | 834 | 1.05 | 253 | 1.51 | 294 |
| Compound 43 | 6.93 | 255 | | | | | | |
| Compound 44 | | | | | | | | |
| Compound 45 | 2.52 | | | | | | | |
| Compound 46A | 7.95 | 394 | | | | | | |
| Compound 46B | 0.964 | 31.7 | | | | | | |
| Compound 47 | | | | | | | | |
| Compound 48 | | | | | | | | |
| Compound 49A | 40.2 | | | | | | | |
| Compound 49B | 2.00 | 144 | | | | | | |
| Compound 50 | 17.2 | 3414 | | | | | | |
| Compound 51 | | | | | | | | |
| Compound 52 | | | | | | | | |
| Compound 53 | 2.10 | 99.8 | 1.13 | 152 | 0.747 | 65.3 | 0.959 | 73.0 |
| Compound 54 | | | | | | | | |
| Compound 55 | | | | | | | | |
| Compound 56 | | | | | | | | |
| Compound 57 | | | | | | | | |
| Compound 59 | | | | | | | | |
| Compound 60 | | | | | | | | |
| Compound 61 | | | | | | | | |
| Compound 62 | | | | | | | | |
| Compound 63 | | | | | | | | |
| Compound 64 | 1.83 | | | | | | | |
| Compound 65 | 1.07 | 14.5 | 0.797 | 16.3 | 1.72 | 4.90 | 1.20 | 5.43 |
| Compound 66 | 1.19 | 18.2 | 1.05 | 18.9 | 1.19 | 5.58 | 0.948 | 7.12 |
| Compound 67 | | | | | | | | |
| Compound 68 | | | | | | | | |
| Compound 69 | | | | | | | | |
| Compound 70 | | | | | | | | |
| Compound 71 | | | | | | | | |
| Compound 73 | 31.6 | | | | | | | |
| Compound 74 | 119 | | | | | | | |
| Compound 75 | | | | | | | | |
| Compound 80 | 126 | | | | | | | |
| Compound 81 | 94.1 | | | | | | | |
| Compound 82 | 2.49 | | | | | | | |
| Compound 83 | 8.04 | | | | | | | |
| Compound 84 | 2.11 | 57.0 | | | | | | |
| Compound 85 | | | | | | | | |
| Compound 86 | 13.2 | | | | | | | |
| Compound 87 | 1.79 | | | | | | | |
| Compound 88 | 28.5 | | | | | | | |
| Compound 89 | 188 | | | | | | | |
| Compound 90 | 19.8 | | | | | | | |
| Compound 84A | 11.4 | 2991 | | | | | | |
| Compound 84B | 2.10 | 20.1 | 1.78 | 113 | 0.630 | 36.4 | 1.26 | 56.4 |
| Compound 93 | 56.0 | | | | | | | |
| Compound 93 | 13.5 | 680 | | | | | | |
| Compound 94 | 105 | | | | | | | |
| Compound 96 | 65.9 | | | | | | | |
| Compound 98 | 318 | >5000 | | | | | | |
| Compound 99 | 34.3 | | | | | | | |
| Compound 100 | 1.23 | 33.9 | 1.05 | 43.3 | 1.41 | 9.25 | 0.844 | 16.7 |
| Compound 101 | 2.56 | 194 | | | | | | |
| Compound 102 | 6.59 | | | | | | | |
| Compound 103 | 8.17 | | | | | | | |
| Compound 104 | 25.2 | | | | | | | |
| Compound 105 | | | | | | | | |
| Compound 106 | 2.62 | | | | | | | |
| Compound 107 | 11.5 | 768 | | | | | | |
| Compound 108 | 119 | | | | | | | |
| Compound 109 | 14.4 | 1259 | | | | | | |
| Compound 109 | 67.9 | | | | | | | |
| Compound 110 | | | | | | | | |
| Compound 111 | | | | | | | | |
| Compound 113 | | | | | | | | |
| Compound 115 | 24.2 | >1000 | 20.4 | >1000 | 7.68 | 445 | 14.8 | >1000 |
| Compound 118 | 29.3 | | | | | | | |
| Compound 120 | 46.7 | | | | | | | |
| Compound 121 | | | | | | | | |
| Compound 123 | | | | | | | | |
| Compound 124 | 5.02 | | | | | | | |
| Compound 125 | 2.16 | | | | | | | |
| Compound 126 | | | | | | | | |
| Compound 127 | | | | | | | | |
| Compound 129 | | | | | | | | |
| Compound 139 | | | | | | | | |
| Compound 147 | 1.87 | | | | | | | |
| Compound 148 | 1.74 | | | | | | | |
| Compound 149 | | | | | | | | |

### 3. Phospho-ERK1/2(THR202/TYR204) HTRF Assay

p-ERK (MAPK pathway) inhibition activity of each of compounds in a variety of K-Ras mutant and K-Ras WT cell lines indicated in Table 23 was evaluated. MKN-1 with K-Ras WT amplification is also a K-Ras dependent cell line.

TABLE 23

| | | Cell | | | |
|---|---|---|---|---|---|
| | | seeding | | | |
| Cell | K-Ras | density, | Culture | Assay | Cell |
| lines | mutation | cells/well | medium | medium | incubator |
| AGS | G12D | 60000 | F-12K, | F-12K, | 37° C., |
| | | | 10% FBS | 0.1% FBS | 5% CO₂ |
| SW620 | G12V | 60000 | L-15, | L-15, | 37° C., |
| | | | 10% FBS | 0.1% FBS | 100% Air |
| NCI- | G12C | 40000 | RPMI 1640, | RPMI 1640, | 37° C., |
| H358 | | | 10% FBS | 0.1% FBS | 5% CO₂ |
| NCI- | G13D | 40000 | RPMI 1640, | RPMI 1640, | 37° C., |
| H747 | | | 10% FBS | 0.1% FBS | 5% CO₂ |
| RERF- | G12A | 30000 | RPMI 1640, | RPMI 1640, | 37° C., |
| LC-Ad1 | | | 10% FBS | 0.1% FBS | 5% CO₂ |
| TCC- | G12R | 20000 | RPMI 1640, | RPMI 1640, | 37° C., |
| PAN2 | | | 10% FBS | 0.1% FBS | 5% CO₂ |
| NCI- | Q61H | 30000 | RPMI 1640, | PMI 1640, | 37° C., |
| H460 | | | 10% FBS | 0.1% FBS | 5% CO₂ |
| MKN-1 | WT | 30000 | RPMI 1640, | RPMI 1640, | 37° C., |
| | | | 10% FBS | 0.1% FBS | 5% CO₂ |

Each of cells in culture medium was seeded in 96-well plates at density indicated in Table 23 and then put in a cell incubator to incubate overnight. The next day, the culture medium was removed and the compound diluted in assay medium was added in each well. After 2 hours incubation in a cell incubator, the assay medium in 96-well plates was removed, then 50 μL of 1× blocking reagent-supplemented lysis buffer (Cisbio) was added and the plates were incubated at 25° C. for 45 min with shaking. 10 μL of cell lysates from the 96-well plates were transferred to a 384-well plate (Greiner) containing 2.5 μL/well HTRF® pre-mixed antibodies (Cisbio 64AERPEH). The plate was incubated 4 hours at 25° C. and then read HTRF signals on Tecan Spark multimode microplate reader. The data were analyzed using a 4-parameter logistic model to calculate IC₅₀ values. The results are shown in the following Table 24:

TABLE 24

| | p-ERK (IC₅₀, nM) | | | | | | | |
|---|---|---|---|---|---|---|---|---|
| Compound | AGS | SW620 | NCI-H358 | NCI-H747 | RERF-LC-Ad1 | TCC-PAN2 | NCI-H460 | MKN-1 |
| Compound 1 | 0.974 | 2.74 | 2.20 | 1.64 | 1.64 | 207 | 7.04 | 1.34 |
| Compound 7 | 0.820 | 2.21 | 1.67 | 0.919 | 1.24 | 31.0 | 2.95 | 3.44 |
| Compound 7B | 0.255 | 1.01 | 0.881 | 0.701 | 0.622 | 13.5 | 1.66 | 1.00 |
| Compound 8A | 21.7 | 37.6 | | | | | | |
| Compound 8B | 5.94 | 8.91 | | | | | | |
| Compound 9 | 66.2 | 15.4 | | | | | | |
| Compound 10 | 6.71 | 10.6 | | | | | | |
| Compound 11 | 14.9 | 18.7 | 2.53 | 17.6 | 8.96 | 560 | 63.8 | 19.5 |
| Compound 12A | 13.0 | 14.0 | | | | | | |
| Compound 12B | 2.00 | 2.08 | 1.19 | 1.96 | 1.01 | 67.8 | 5.43 | 1.76 |
| Compound 13A | 44.4 | 42.3 | | | | | | |
| Compound 13B | 1.10 | 5.82 | | | | | | |
| Compound 14A | 537 | 890 | | | | | | |
| Compound 14B | 22.9 | 112 | | | | | | |
| Compound 15B | 13.9 | 30.1 | | | | | | |
| Compound 16 | 2.31 | 19.4 | | | | | | |
| Compound 16A | 42.6 | 40.5 | | | | | | |
| Compound 16B | 1.44 | 5.07 | 2.10 | 3.56 | 1.75 | 97.6 | 18.1 | 2.97 |
| Compound 18 | 5.99 | 2.10 | | | | | | |
| Compound 19 | 47.9 | 52.5 | | | | | | |
| Compound 20 | 53.0 | 29.3 | | 28.2 | | | | |
| Compound 21 | 10.2 | 135 | | 60.7 | | | | |
| Compound 22 | 70.7 | 13.2 | | | | | | |
| Compound 23 | 33.8 | 138 | | | | | | |
| Compound 24 | 23.1 | 4.39 | | | | | | |
| Compound 25 | 7.43 | 3.41 | 4.33 | 9.12 | 4.01 | 601 | 18.4 | 6.63 |
| Compound 27 | 65.1 | 144 | | | | | | |
| Compound 28B | 1.33 | 3.36 | | | | | | |
| Compound 29 | 3.30 | 7.74 | | | | | | |
| Compound 30 | 2.04 | 3.97 | | | | | | |
| Compound 31 | 3.62 | 12.8 | | | | | | |
| Compound 32A | 10.4 | 30.0 | | | | | | |
| Compound 32B | 12.1 | 17.1 | | | | | | |
| Compound 33 | 1.36 | 4.00 | 1.73 | 0.902 | 1.66 | 94.0 | 2.26 | 1.44 |
| Compound 34 | 19.4 | 40.0 | | | | | | |
| Compound 35 | 1.42 | 11.9 | | | | | | |
| Compound 36 | 12.4 | 48.8 | | | | | | |
| Compound 37 | 49.2 | 92.0 | | | | | | |
| Compound 38 | 2.16 | 7.72 | 2.23 | 3.17 | 1.97 | 218 | 14.9 | 10.6 |
| Compound 39 | 1.15 | 3.36 | 1.38 | 2.70 | 1.67 | 156 | 9.92 | 9.92 |
| Compound 40 | 5.68 | 14.1 | | | | | | |
| Compound 42 | 10.1 | 3.02 | 7.37 | 2.56 | 2.20 | ~1000 | 10.0 | 7.55 |
| Compound 43 | 101 | 21.8 | | | | | | |
| Compound 46A | 83.9 | 321 | | | | | | |
| Compound 46B | 15.0 | 45.7 | | | | | | |
| Compound 47 | 17.7 | 40.9 | | | | | | |
| Compound 49A | | 98.6 | | | | | | |
| Compound 49B | 62.2 | 19.9 | | | | | | |
| Compound 50 | | 121 | | | | | | |
| Compound 51 | 2.27 | 8.50 | | | | | | |
| Compound 52 | 9.66 | 2.43 | | | | | | |
| Compound 53 | 2.66 | 6.18 | 3.86 | 6.93 | 3.12 | 456 | 17.5 | 9.74 |
| Compound 64 | 4.32 | 10.9 | | | | | | |
| Compound 65 | 0.810 | 4.31 | 1.24 | 2.94 | 1.22 | 74.9 | 7.82 | 3.71 |
| Compound 66 | 1.15 | 2.74 | 3.48 | 4.41 | 1.32 | 121 | 12.8 | 7.81 |
| Compound 82 | 2.93 | 13.8 | | | | | | |
| Compound 83 | 640 | 819 | | | | | | |
| Compound 84 | 5.45 | 9.17 | | | | | | |
| Compound 85 | 140 | 43.6 | | | | | | |
| Compound 87 | 1.50 | 5.98 | | | | | | |
| Compound 84A | 29.1 | 19.4 | | | | | | |
| Compound 84B | 2.76 | 5.74 | 3.78 | 4.68 | 2.23 | 133 | 21.8 | 2.15 |
| Compound 93 | | 51.6 | | | | | | |
| Compound 94 | | 275 | | | | | | |
| Compound 100 | 2.63 | 14.3 | 2.04 | 6.66 | 1.61 | 104 | 17.7 | 3.57 |
| Compound 101 | 7.70 | 95.6 | | | | | | |
| Compound 102 | 192 | 47.1 | | | | | | |
| Compound 103 | | 187 | | | | | | |
| Compound 105 | 23.0 | 34.8 | | | | | | |
| Compound 106 | 39.4 | 21.5 | | | | | | |
| Compound 107 | 66.1 | 40.2 | | | | | | |
| Compound 109 | 203 | 238 | | | | | | |
| Compound 110 | 44.3 | 62.2 | | | | | | |
| Compound 111 | 17.4 | 84.0 | | | | | | |
| Compound 113 | 1.83 | 10.4 | | | | | | |
| Compound 115 | 46.5 | 27.9 | | | | | | |
| Compound 118 | 113 | 110 | | | | | | |
| Compound 121 | 5.47 | 34.8 | | | | | | |
| Compound 123 | 517 | 803 | | | | | | |
| Compound 124 | 34.2 | 21.8 | | | | | | |
| Compound 125 | 9.51 | 22.5 | | | | | | |
| Compound 129 | 83.1 | | | | | | | |
| Compound 147 | 4.01 | 11.2 | | 21.4 | | | | |
| Compound 148 | 4.57 | 13.0 | | 12.9 | | | | |
| Compound 149 | 6.85 | 14.9 | | | | | | |

### 4. Cell Growth Inhibition Assay

The cell growth inhibition activity of each of compounds was tested by performing cell growth inhibition assays on a variety of K-Ras mutant and K-Ras WT cell lines indicated in Table 25.

TABLE 25

| | | Cell | | | |
|---|---|---|---|---|---|
| | | seeding | | | |
| Cell | K-Ras | density, | Culture | Assay | Cell |
| lines | mutation | cells/well | medium | format | incubator |
| AGS | G12D | 500 | F-12K, | 2D | 37° C., |
| | | | 10% FBS | | 5% CO₂ |
| AsPC-1 | G12D | 1000 | RPMI 1640, | 2D | 37° C., |
| | | | 10% FBS | | 5% CO₂ |
| SW620 | G12V | 1500 | L-15, | 2D | 37° C., |
| | | | 10% FBS | | 100% Air |
| Capan-2 | G12V | 1500 | McCoy′5A, | 3D | 37° C., |
| | | | 10% FBS | | 5% CO₂ |
| NCI- | G12C | 1500 | RPMI 1640, | 3D | 37° C., |
| H358 | | | 10% FBS | | 5% CO₂ |
| NCI- | G13D | 1500 | RPMI 1640, | 3D | 37° C., |
| H747 | | | 10% FBS | | 5% CO₂ |
| RERF- | G12A | 3000 | RPMI 1640, | 3D | 37° C., |
| LC-Ad1 | | | 10% FBS | | 5% CO₂ |
| TCC- | G12R | 500 | RPMI 1640, | 3D | 37° C., |
| PAN2 | | | 10% FBS | | 5% CO₂ |
| NCI- | Q61H | 300 | RPMI 1640, | 3D | 37° C., |
| H460 | | | 10% FBS | | 5% CO₂ |
| MKN-1 | WT | 1500 | RPMI 1640, | 3D | 37° C., |
| | | | 10% FBS | | 5% CO₂ |

### 2D Cell Growth Inhibition Assays

Each of cells in culture medium was plated in TC-treated 96-well plates at a density indicated in Table 25 and incubated in a cell incubator overnight. The next day, each of compounds was diluted in culture medium and added to the plates. After 6 days incubation in cell incubator, the cell viability was detected by CellTiter-Glo® Cell Viability Assay kit (Promega). Luminescent signals were read on Tecan Spark multimode microplate reader and analyzed using a 4-parameter logistic model to calculate absolute IC₅₀ values. The results are shown in the following Table 26.

### 3D Cell Growth Inhibition Assays

Each of cells in culture medium was plated in ultra-low attachment-coated 96-well plates at a density indicated in Table 25 and incubated in a cell incubator overnight. The next day, each of compounds was diluted in culture medium and added to the plates. After 6 days incubation in cell incubator, the cell viability was detected by CellTiter-Glo® 3D Cell Viability Assay kit (Promega). Luminescent signals were read on Tecan Spark multimode microplate reader and analyzed using a 4-parameter logistic model to calculate absolute IC₅₀ values. The results are shown in the following Table 26.

TABLE 26

| | Cell Growth Inhibition, IC₅₀(nM) | | | | | | | | | |
|---|---|---|---|---|---|---|---|---|---|---|
| Compound | AGS | AsPC-1 | SW620 | Capan-2 | NCI-H358 | NCI-H747 | RERF-LC-Ad1 | TCC-PAN2 | NCI-H460 | MKN-1 |
| Compound 1 | 2.45 | 43.8 | 4.68 | 28.7 | 18.6 | 5.65 | 20.6 | 189 | 75.1 | 8.23 |
| Compound 2 | 178 | 293 | 182 | | | | | | | |
| Compound 7 | 0.778 | 12.8 | 1.33 | 7.34 | 2.32 | 3.17 | 5.20 | 165 | 39.8 | 3.46 |
| Compound 7A | 175 | 1508 | 846 | | | | | | | |
| Compound 7B | 0.423 | 2.73 | 0.858 | 2.91 | 2.57 | 0.807 | 2.52 | 41.0 | 17.9 | 3.70 |
| Compound 8A | 98.1 | 608 | 133 | | | | | | | |
| Compound 8B | 27.4 | 153 | 19.6 | | | | | | | |
| Compound 9 | 186 | 586 | 171 | | | | | | | |
| Compound 10 | 26.2 | 228 | 10.5 | | | | | | | |
| Compound 11 | 40.6 | 380 | 34.1 | 311 | 62.6 | 56.0 | 150 | >1000 | 866 | 28.1 |
| Compound 12A | 24.2 | 301 | 25.2 | | | | | | | |
| Compound 12B | 2.17 | 37.7 | 3.11 | 52.3 | 3.47 | 3.84 | 7.16 | 184 | 83.0 | 9.09 |
| Compound 13A | 122 | 680 | 93.6 | | | | | | | |
| Compound 13B | 6.35 | 43.0 | 5.48 | | | | | | | |
| Compound 14A | 610 | 6194 | >1000 | | | | | | | |
| Compound 14B | 49.7 | 708 | 207 | | | | | | | |
| Compound 15B | 30.4 | 465 | 59.6 | | | | | | | |
| Compound 16 | 18.6 | 133 | 39.5 | | | | | | | |
| Compound 16A | 242 | 1384 | 191 | | | | | | | |
| Compound 16B | 5.82 | 53.0 | 8.77 | 121 | 13.7 | 21.6 | 30.9 | 319 | 296 | 20.9 |
| Compound 18 | 7.96 | 95.7 | 5.68 | | | | | | | |
| Compound 19 | 5514 | 2372 | 119 | | | | | | | |
| Compound 20 | 203 | 538 | 139 | | | 339 | | | | |
| Compound 21 | 51.9 | 1352 | 61.4 | | | 126 | | | | |
| Compound 22 | 175 | 782 | 64.3 | | | | | | | |
| Compound 23 | 40.1 | 693 | 196 | | | | | | | |
| Compound 24 | 42.4 | 381 | 15.3 | | | | | | | |
| Compound 25 | 24.2 | 129 | 18.9 | 272 | 38.7 | 28.5 | 64.0 | >1000 | 278 | 41.7 |
| Compound 27 | 216 | 904 | 90.7 | | | | | | | |
| Compound 28B | 2.23 | 18.2 | 6.63 | | | | | | | |
| Compound 29 | 8.22 | 53.6 | 4.62 | | | | | | | |
| Compound 31 | 5.92 | 60.9 | 22.6 | | | | | | | |
| Compound 32A | 20.6 | 150 | 28.6 | | | | | | | |
| Compound 32B | 18.6 | 135 | 27.9 | | | | | | | |
| Compound 33 | 4.19 | 33.6 | 2.05 | 27.4 | 3.81 | 1.34 | 3.67 | 117 | 24.6 | 3.11 |
| Compound 34 | 75.4 | 323 | 205 | | | | | | | |
| Compound 35 | 5.63 | 88.5 | 9.20 | | | | | | | |
| Compound 36 | 37.3 | 734 | 112 | | | | | | | |
| Compound 37 | 278 | 629 | 330 | | | | | | | |
| Compound 38 | 2.73 | 77.3 | 13.1 | 57.8 | 13.6 | 10.4 | 60.9 | 1000 | 159 | 10.0 |
| Compound 39 | 2.09 | 61.2 | 9.66 | 75.4 | 13.9 | 7.27 | 31.9 | 949 | 125 | 7.34 |
| Compound 40 | 9.24 | 91.3 | 24.8 | | | | | | | |
| Compound 41 | 62.2 | 238 | 88.8 | | | | | | | |
| Compound 42 | 39.1 | 135 | 4.18 | 49.2 | 12.6 | 12.4 | 21.2 | 428 | 113 | 8.69 |
| Compound 43 | 248 | 1849 | 76.3 | | | | | | | |
| Compound 46A | 187 | 2866 | 630 | | | | | | | |
| Compound 46B | 20.6 | 446 | 50.1 | | | | | | | |
| Compound 47 | 51.7 | 106 | 41.6 | | | | | | | |
| Compound 49B | 41.2 | 428 | 54.5 | | | | | | | |
| Compound 51 | 9.66 | 74.2 | 10.3 | | | | | | | |
| Compound 52 | 8.11 | 40.4 | 5.66 | | | | | | | |
| Compound 53 | 9.17 | 88.2 | 19.4 | 153 | 47.2 | 34.0 | 166 | 1000 | 24 | 25.7 |
| Compound 64 | 24.5 | 130 | 41.3 | | 61.6 | | | | | |
| Compound 65 | 1.61 | 29.2 | 3.03 | 7.15 | 14.8 | 9.70 | 25.9 | 275 | 37.2 | 11.0 |
| Compound 66 | 3.86 | 50.3 | 7.33 | 12.3 | 26.0 | 10.4 | 26.6 | 621 | 53.2 | 23.9 |
| Compound 82 | 15.5 | 226 | 116 | | | | | | | |
| Compound 84 | 14.3 | 120 | 15.7 | | | | | | | |
| Compound 85 | 143 | 860 | 130 | | | | | | | |
| Compound 87 | 3.41 | 32.8 | 27.2 | | | | | | | |
| Compound 84A | 203 | 1101 | 60.6 | | | | | | | |
| Compound 84B | 10.2 | 136 | 10.9 | 175 | 18.2 | 20.2 | 80.4 | 999 | 461 | 28.8 |
| Compound 93 | | | 182 | | | | | | | |
| Compound 100 | 5.40 | 67.2 | 8.64 | 76.9 | 12.1 | 16.3 | 35.5 | 720 | 139 | 21.1 |
| Compound 101 | 25.5 | 218 | 120 | | | | | | | |
| Compound 103 | | | 109 | | | | | | | |
| Compound 105 | 53.6 | 326 | 104 | | | | | | | |
| Compound 106 | 98.1 | 681 | 66.4 | | | | | | | |
| Compound 107 | 504 | 885 | 185 | | | | | | | |
| Compound 110 | | | 81.0 | | | | | | | |
| Compound 111 | 86.0 | 390 | 73.8 | | | | | | | |
| Compound 113 | 9.76 | 121 | 22.4 | | | | | | | |
| Compound 115 | 133 | 237 | 163 | | | | | | | |
| Compound 118 | 184 | 1416 | 343 | | | | | | | |
| Compound 121 | 25.4 | 320 | 91.0 | | | | | | | |
| Compound 123 | 8.51 | 2801 | 525 | | | | | | | |
| Compound 124 | 80.8 | 784 | 45.7 | | | | | | | |
| Compound 125 | 18.4 | 124 | 31.6 | | | | | | | |
| Compound 129 | 521 | 1074 | | | | | | | | |
| Compound 147 | 23.7 | 142 | 17.5 | | | 26.6 | | | | |
| Compound 148 | 9.03 | 72.5 | 13.9 | | 11.0 | 10.7 | | | | |
| Compound 149 | 22.9 | 187 | 26.2 | | | | | | | |

What is claimed is:

1-90. (canceled)

91. A compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof: 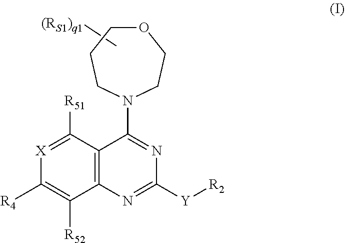
Wherein,
X is selected from N or CR₅₃;
R_{S1} at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein, said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S1} together with the carbon atom to which they are both attached form 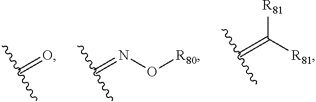
a 3-20 membered carbocyclic ring or a 3-20 heterocyclic ring; wherein, said 3-20 membered carbocylic ring or 3-20 heterocyclic ring is optionally substituted with one or more R₁₆ₐ;
Optionally, two adjacent R_{S1} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R_{16b};
Optionally, two nonadjacent R_{S1} are connected together to form a C₀₋₆ alkylene bridge, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R_{16c};
q₁ is independently selected from 0, 1, 2, 3, 4, 5, 6, 7, 8, 9 or 10;
R₂ is selected from -L₅-(3-12 membered heterocyclyl), -L₅-(3-12 membered cycloalkyl), -L₅-(6-12 member aryl), -L₅-(5-12 membered heteroaryl), -L₅-N(R₇₅)₂, 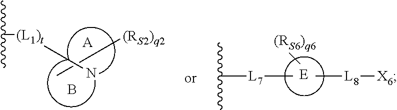
Each of L₅ at each occurrence is independently selected from a bond or C₁₋₁₀ alkylene optionally substituted with one or more R₁₆ₙ;
Said 3-12 membered heterocyclyl in -L₅-(3-12 membered heterocyclyl) is optionally substituted with one or more R₁₆ₒ;
Said 3-12 membered cycloalkyl in -L₅-(3-12 membered cycloalkyl) is optionally substituted with one or more R₁₆ₒ;
Said 6-12 member aryl in -L₅-(6-12 member aryl) is optionally substituted with one or more R₁₆ₒ;
Said 5-12 membered heteroaryl in -L₅-(5-12 membered heteroaryl) is optionally substituted with one or more R₁₆ₒ;
L₇ is selected from a bond or C₁₋₁₀ alkylene optionally substituted with one or more R_{16q};
L₈ is selected from a bond or C₁₋₁₀ alkylene optionally substituted with one or more R₁₆ᵣ;
Ring E is selected from a 3-10 membered carbocyclic ring or a 3-10 membered heterocyclic ring; wherein the moiety of -L₇- and -L₅-X₆ are attached to the same atom or different atoms of the ring E;
X₆ is selected from —N(R₆₅)₂, —OR₆₅, —SR₆₅, 3-10 membered heterocyclyl, or 5-10 membered heteroaryl, wherein said 3-10 membered heterocyclyl or 5-10 membered heteroaryl is optionally independently substituted with one or more R₁₆ₐ;
Y is selected from a bond, —C(R₆₃)₂—, —R₆₃C═CR₆₃—, —C≡C—, —C(═O)—, —O—, —NR₆₃—, —S—, —S(═O)—, —S(═O)₂—, —PR₆₃—, —P(═O)R₆₄—, —C(═O)O—, —OC(═O)—, —C(═O)NR₆₃—, —NR₆₃C(═O)—, —S(═O)O—, —OS(═O)—, —S(═O)₂O—, —OS(═O)₂—, —S(═O)NR₆₃—, —NR₆₃S(═O)—, —S(═O)₂NR₆₃—, —NR₆₃S(═O)₂—, —OC(═O)O—, —NR₆₃C(═O)O—, —OC(═O)NR₆₃— or —NR₆₃C(═O)NR₆₃—;
L₁ is selected from a bond, —C(R₆₅)₂—, —R₆₅C═CR₆₅—, —C≡C—, —C(═O)—, —O—, —NR₆₅—, —S—, —S(═O)—, —S(═O)₂—, —PR₆₅—, —P(═O)R₆₆—, —C(═O)O—, —OC(═O)—, —C(═O)NR₆₅—, —NR₆₅C(═O)—, —S(═O)O—, —OS(═O)—, —S(═O)₂O—, —OS(═O)₂—, —S(═O)NR₆₅—, —NR₆₅S(═O)—, —S(═O)₂NR₆₅—, —NR₆₅S(═O)₂—, —OC(═O)O—, —NR₆₅C(═O)O—, —OC(═O)NR₆₅— or —NR₆₅C(═O)NR₆₅—;
t is selected from 0, 1, 2, 3, 4, 5, 6, 7, 8, 9 or 10;
Ring A or ring B is a 3-20 membered heterocyclic ring which is optionally further contains 1, 2, 3, 4, 5, 6, 7, 8, 9, or 10 ring members selected from —N—, —O—, —S—, —C(═O)—, —S(═O)—, —S(═O)₂—, —S(═O)O—, —OS(═O)—, —S(═O)₂O—, —OS(═O)₂—, —S(═O)NH—, —NHS(═O)—, —S(═O)₂NH—, —NHS(═O)₂—; or a 3-20 membered heteroaryl ring which is optionally further contains 1, 2, 3, 4, 5, 6, 7, 8, 9, or 10 ring members, selected from —N—, —O— or —S—;
Each of R_{S2} at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₇)₂, —OR₆₇, —SR₆₇, —S(═O)R₆₈, —S(═O)₂R₆₈, —C(═O)R₆₈, —C(═O)OR₆₇, —OC(═O)R₆₈, —C(═O)N(R₆₇)₂, —NR₆₇C(═O)R₆₈, —OC(═O)OR₆₇, —NR₆₇C(═O)OR₆₇, —NR₆₇C(═S)OR₆₇, —OC(═O)N(R₆₇)₂, —NR₆₇C(═O)N(R₆₇)₂, —S(═O)OR₆₇, —OS(═O)R₆₇, —S(═O)N(R₆₇)₂, —NR₆₇S(═O)R₆₈, —S(═O)₂OR₆₇, —OS(═O)₂R₆₈, —S(═O)₂N(R₆₇)₂, —NR₆₇S(═O)₂R₆₈, —OS(═O)₂OR₆₇, —NR₆₇S(═O)₂OR₆₇, —OS(═O)₂N(R₆₇)₂, —NR₆₇S(═O)₂N(R₆₇)₂, —P(R₆₇)₂, —P(═O)(R₆₈)₂, 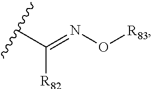
3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₇)₂, —OR₆₂, —SR₆₇, —S(═O)R₆₈, —S(═O)₂R₆₈, —C(═O)R₆₈, —C(═O)OR₆₇, —OC(═O)R₆₈, —C(═O)N(R₆₇)₂, —NR₆₇C(═O)R₆₈, —OC(═O)OR₆₇, —NR₆₇C(═O)OR₆₇, —NR₆₇C(═S)OR₆₇, —OC(═O)N(R₆₇)₂, —NR₆₇C(═O)N(R₆₇)₂, —S(═O)OR₆₇, —OS(═O)R₆₇, —S(═O)N(R₆₇)₂, —NR₆₇S(═O)R₆₈, —S(═O)₂OR₆₇, —OS(═O)₂R₆₈, —S(═O)₂N(R₆₇)₂, —NR₆₇S(═O)₂R₆₈, —OS(═O)₂OR₆₇, —NR₆₇S(═O)₂OR₆₇, —OS(═O)₂N(R₆₇)₂, —NR₆₇S(═O)₂N(R₆₇)₂, —P(R₆₇)₂, —P(═O)(R₆₈)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S2} together with the carbon atom to which they are both attached form 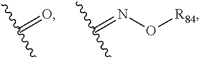
a 3-10 membered carbocyclic ring or a 3-10 heterocyclic ring; wherein, sais 3-10 membered carbocyclic ring or 3-10 heterocyclic ring is optionally substituted with one or more R_{16d};
Optionally, two adjacent R_{S2} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₁₆ₑ;
Optionally, two nonadjacent R_{S2} are connected together to form a C₀₋₆ alkylene bridge, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R_{16f};
q₂ is selected from 0, 1, 2, 3, 4, 5, 6, 7, 8, 9 or 10;
Each of R_{S6} at each occurrence is independently selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₃)₂, —OR₇₃, —SR₇₃, —S(═O)R₇₄, —S(═O)₂R₇₄, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₃, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₃)₂, —OR₇₃, —SR₇₃, —S(═O)R₇₄, —S(═O)₂R₇₃, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₄, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
q₆ is selected from 0, 1, 2, 3, 4, 5 or 6;
R₄ is selected from 6-20 membered aryl, 5-20 membered heteroaryl 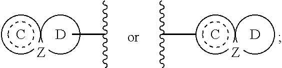
wherein said 6-20 membered aryl, 5-20 membered heteroaryl, 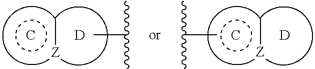
is optionally independently substituted with one or more R₄₁;
Z at each occurrence is independently selected from C or N;
Ring C at each occurrence is independently selected from a 6 membered aryl ring or a 5-6 membered heteroaryl ring and ring D at each occurrence is a 3-10 membered carbocyclic ring or a 3-10 membered heterocyclic ring when Z is selected from C;
Ring C at each occurrence is selected from a 5-6 membered heteroaryl ring and ring D at each occurrence is a 3-10 membered heterocyclic ring when Z is selected from N;
R₄₁ at each occurrence is independently selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₉)₂, —OR₆₉, —SR₆₉, —S(═O)R₇₀, —S(═O)₂R₇₀, —C(═O)R₇₀, —C(═O)OR₆₉, —OC(═O)R₇₀, —C(═O)N(R₆₉)₂, —NR₆₉C(═O)R₇₀, —OC(═O)OR₆₉, —NR₆₉C(═O)OR₆₉, —OC(═O)N(R₆₉)₂, —NR₆₉C(═O)N(R₆₉)₂, —S(═O)OR₆₉, —OS(═O)R₇₀, —S(═O)N(R₆₉)₂, —NR₆₉S(═O)R₇₀, —S(═O)₂OR₆₉, —OS(═O)₂R₇₀, —S(═O)₂N(R₆₉)₂, —NR₆₉S(═O)₂R₇₀, —OS(═O)₂OR₆₉, —NR₆₉S(═O)₂OR₆₉, —OS(═O)₂N(R₆₉)₂, —NR₆₉S(═O)₂N(R₆₉)₂, —P(R₆₉)₂, —P(═O)(R₇₀)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₆₉)₂, —OR₆₉, —SR₆₉, —S(═O)R₇₀, —S(═O)₂R₇₀, —C(═O)R₇₀, —C(═O)OR₆₉, —OC(═O)R₇₀, —C(═O)N(R₆₉)₂, —NR₆₉C(═O)R₇₀, —OC(═O)OR₆₉, —NR₆₉C(═O)OR₆₉, —OC(═O)N(R₆₉)₂, —NR₆₉C(═O)N(R₆₉)₂, —S(═O)OR₆₉, —OS(═O)R₇₀, —S(═O)N(R₆₉)₂, —NR₆₉S(═O)R₇₀, —S(═O)₂OR₆₉, —OS(═O)₂R₇₀, —S(═O)₂N(R₆₉)₂, —NR₆₉S(═O)₂R₇₀, —OS(═O)₂OR₆₉, —NR₆₉S(═O)₂OR₆₉, —OS(═O)₂N(R₆₉)₂, —NR₆₉S(═O)₂N(R₆₉)₂, —P(R₆₉)₂, —P(═O)(R₇₀)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Each of (R₅₁, R₅₂ and R₅₃) is independently selected from hydrogen, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, —N(R₇₁)₂, —OR₇₁, —SR₇₁, —S(═O)R₇₂, —S(═O)₂R₇₂, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₁, —OS(═O)₂N(R₇₁)₂, —NR₇₁S(═O)₂N(R₇₁)₂, —P(R₇₁)₂, —P(═O)(R₇₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₇₁)₂, —OR₇₁, —SR₇₁, —S(═O)R₇₂, —S(═O)₂R₇₂, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₁, —OS(═O)₂N(R₇₁)₂, —NR₇₁S(═O)₂N(R₇₁)₂, —P(R₇₁)₂, —P(═O)(R₇₂)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₆₁, R₆₂, R₆₃, R₆₄, R₆₅, R₆₆, R₆₇, R₆₈, R₆₉, R₇₀, R₇₁, R₇₂, R₇₃, R₇₄, R₇₅, R₈₀, R₈₁, R₈₂, R₈₃, and R₈₄) at each occurrence is independently selected from hydrogen, —C₁₋₁₀alkyl, haloC₁₋₁₀alkyl, haloC₁₋₁₀alkoxy, —C₂₋₁₀alkenyl, —C₂₋₁₀alkynyl, —CN, —NO₂, —N₃, oxo, —N(Rₐ)₂, —ORₐ, —SRₐ, —S(═O)R_{b}, —S(═O)₂R_{b}, —C(═O)R_{b}, —C(═O)ORₐ, —OC(═O)R_{b}, —C(═O)N(Rₐ)₂, —NRₐC(═O)R_{b}, —OC(═O)ORₐ, —NR_{c}C(═O)ORₐ, —OC(═O)N(Rₐ)₂, —NRₐC(═O)N(Rₐ)₂, —S(═O)ORₐ, —OS(═O)R_{b}, —S(═O)N(Rₐ)₂, —NRₐS(═O)R_{b}, —S(═O)₂ORₐ, —OS(═O)₂R_{b}, —S(═O)₂N(Rₐ)₂, —NRₐS(═O)₂R_{b}, —OS(═O)₂ORₐ, —NRₐS(═O)₂ORₐ, —OS(═O)₂NRₐ, —NRₐS(═O)₂N(Rₐ)₂, —P(Rₐ)₂, —P(═O)(R_{b})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR_{c}, —SR, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, each of (two R₆₁, two R₆₂, two R₆₉, two R₇₁, two R₇₃, two R₇₅, two Rₐ and two R_{c}) independently together with the nitrogen atom to which they are both attached forms a 3-20 membered heterocyclic ring or a 5-10 membered heteroaryl ring, wherein, said 3-20 membered heterocyclic ring or 5-10 membered heteroaryl ring is optionally independently substituted with one or more R_{16g};
Optionally, each of (two R₆₃ and two R₆₅) independently together with the carbon atom to which they are both or independently attached forms a 3-20 membered carbocyclic ring or 3-20 membered heterocyclic ring, wherein, said 3-20 membered carbocyclic ring or 3-20 membered heterocyclic ring is optionally independently substituted with one or more R₁₆ₕ;
Each of (Rₐ, R_{b}, R_{c} and R_{d}) at each occurrence is independently selected from hydrogen, —C₁₋₆alkyl, haloC₁₋₆ alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl;
wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₁₆ᵢ;
Each of (R₁₆ₐ, R_{16b}, R_{16c}, R_{16d}, R₁₆ₑ, R_{16f}, R_{16g}, R₁₆ₕ, R₁₆ᵢ, R₁₆ₙ, R₁₆ₒ, R_{16q}, R₁₆ᵣ and R₁₆ₛ) at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆ alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃ alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃ alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

92. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the compound is of formula (I-1): 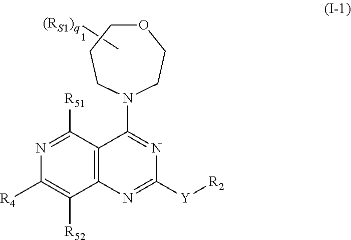
Wherein,
R₂ is selected from

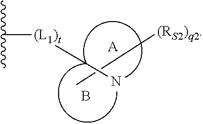

93. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein,
R_{S1} at each occurrence is independently selected from —Cl, —F, —Br, —CH₃, —CH₂CH₃, —CH(CH₃)₂, —C(CH₃)₃, 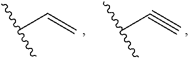
—CH₂F, —CHF₂, —CF₃, —CHFCH₃, —CF₂CH₃, —CN, —NH₂, —NH(CH₃), —N(CH₃)₂, —OH, —O—CH₃, —O—CH₂CH₃, —O—CH(CH₃)₂, —C(═O)CH₃, —C(═O)(CH₂CH₃), —C(═O)—CH(CH₃)₂, —C(═O)(CF₃), —C(═O)NH₂, —C(═O)NH(CH₃), —CH₂—OH, —CH₂CH₂—OH, —CH(CH₃)—OH, —CH₂—CN, —CH₂CH₂—CN, —CH(CH₃)—CN, —O—CH₃—O—CH₃, —O—CH₂CH₃—O—CH₃, —N(CH₃)—O—CH₃, —N(CH₂CH₃)—O—CH₃, 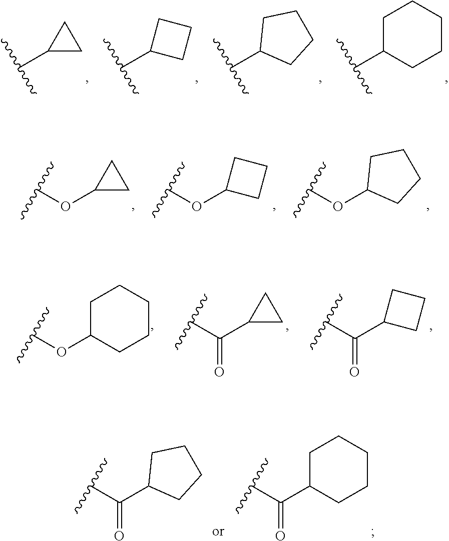
Optionally, two R_{S1} together with the carbon atom to which they are both attached form 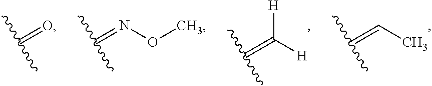
a 3 membered carbocyclic ring, or a 4 membered carbocyclic ring.

94. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein,
R_{S1} at each occurrence is independently selected from —Cl, —F, —Br, —CH₃, —CH₂CH₃, —CH(CH₃)₂, —C(CH₃)₃, —CN, —CH₂—CN, —CH₂CH₂—CN or —CH(CH₃)—CN.

95. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, two adjacent R_{S1} together with the carbon atoms to which they are respectively attached form a 3 membered carbocyclic ring; 4 membered carbocyclic ring or 6 membered heterocyclic ring containing 2 ring members selected from O.

96. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the moiety of 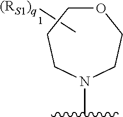
is selected from

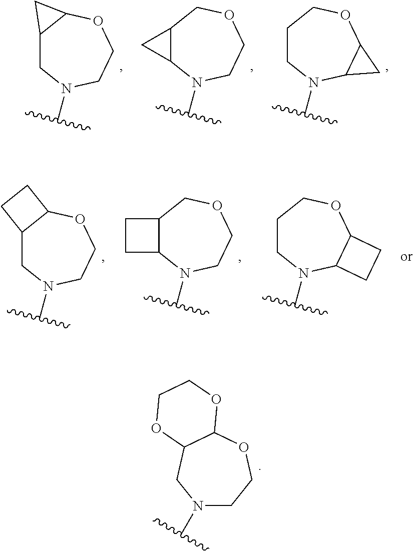

97. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the moiety of 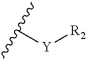
is selected from

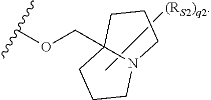

98. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein the compound is selected from any one of the following formulas: 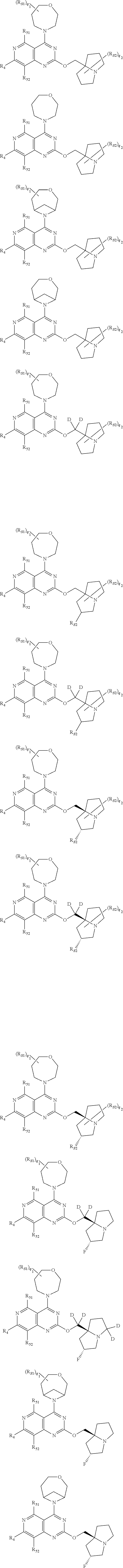
p is selected from 0, 1, 2 or 3.

99. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein,
R_{S2} at each occurrence is independently selected from halogen, —C₁₋₆alkyl, haloC₁₋₆alkyl, haloC₁₋₆alkoxy, —C₂₋₃alkenyl, —CN, —N(R₆₇)₂, —OR₆₇, —SR₆₇, —C(═O)R₆₈, —C(═O)OR₆₇, —OC(═O)R₆₈, —C(═O)N(R₆₇)₂, —NR₆₇C(═O)R₆₈, —OC(═O)OR₆₇, —NR₆₇C(═O)OR₆₇, —OC(═O)N(R₆₇)₂, —NR₆₇C(═O)N(R₆₇)₂, 3-8 membered cycloalkyl, 4-8 membered heterocyclyl containing 1, 2 or 3 heteroatoms selected from N, O or S or 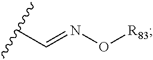
wherein, said —C₁₋₆alkyl is substituted with 1, 2 or 3 substituents selected from halogen, —C₁₋₆alkyl, haloC₁₋₆alkyl, haloC₁₋₆alkoxy, —CN, oxo, —N(R₆₇)₂, —OR₆₇, —C(═O)R₆₈, —C(═O)OR₆₇, —OC(═O)R₆₇, —C(═O)N(R₆₇)₂, —NR₆₇C(═O)R₆₈, —OC(═O)OR₆₇, —NR₆₇C(═O)OR₆₇, —OC(═O)N(R₆₇)₂, —OC(═S)N(R₆₇)₂, —NR₆₇C(═O)N(R₆₇)₂, —NR₆₇S(═O)₂R₆₈, 3-6 membered cycloalkyl or 4-6 membered heterocyclyl; said 4-8 membered heterocyclyl is substituted with 1, 2 or 3 substituents selected from —OR₆₇; said haloC₁₋₆alkyl is substituted with 1, 2 or 3 substituents selected from —OR₆₇ or —C(═O)OR₆₇; said —C₂₋₃alkenyl is substituted with 1 substituents selected from —C(═O)(R₆₇)₂;
Optionally, two R_{S2} together with the carbon atom to which they are both attached form 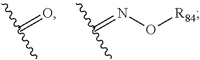
Each of (R₆₇ or R₆₈) is independently selected from hydrogen; —C₁₋₆alkyl; haloC₁₋₆alkyl; 5 membered heteroaryl; cyclopropyl; cyclopentyl; cyclohexyl; 5 membered heterocyclyl; 6 membered heterocyclyl; 5-membered heteroaryl; 6 membered heteroaryl; or —C₁₋₆alkyl substituted with 1, or 2 substituents selected from —OC₁₋₆alkyl, —NHC₁₋₆alkyl, —N(C₁₋₆alkyl)₂ or —C(═O)N(C₁₋₆alkyl)₂; wherein said 5 membered heteroaryl, cyclopropyl, cyclopentyl, cyclohexyl, 5 membered heterocyclyl, 6 membered heterocyclyl, 5-membered heteroaryl or 6 membered heteroaryl is optionally substituted with 1 or 2 substitutents selected from —C₁₋₃alkyl, —OH, —CN, —NH₂, —NH(C₁₋₃alkyl), —N(C₁₋₃alkyl)₂, —OC₁₋₃alkyl or cyclopropyl;
Optionally, two R₆₇ together with the nitrogen atom to which they are both attached form a 3-6 membered heterocyclic ring;
q₂ is selected from 0, 1, 2, 3, 4, 5 or 6; preferably, q₂ is selected from 0, 1, 2, or 3; more preferably, q₂ is selected from 1.

100. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the moiety of 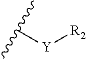
is selected from any moiety in the Table 3 as shown in the description.

101. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the moiety of 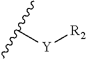
is selected from any moiety in the Table 4 as shown in the description.

102. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, R₄ is selected from any moiety in the Table 6 as shown in the description, wherein, each moiety in the Table 6 is independently optionally substituted with 1, 2, 3, 4, 5 or 6 R₄₁.

103. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the compound is selected from any one of the following formulas: [chemical structure]
R₁₆ is selected from hydrogen or deuterium;
s is selected from 0, 1, 2, 3, 4, 5 or 6;
t is selected from 0, 1, 2, 3 or 4.

104. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, R₄₁ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)H, —S(═O)(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, —NH₂, —SH, 3-6 membered cycloalkyl or 3-6 membered heterocyclyl is independently optionally substituted with 1, 2 or 3 R₄₂;
Each of R₄₂ is independently selected from —F; —C₁₋₃alkyl; haloC₁₋₃alkyl; —CN; —OH; —NH₂; —NH(C₁₋₃alkyl); —N(C₁₋₃alkyl)₂; —OC₁₋₃alkyl; 3-6 membered cycloalkyl; or —C₁₋₃alkyl substituted with 1, 2 or 3 substituents selected from —F, haloC₁₋₃alkyl, —CN, —OH, —NH₂, —NH(C₁₋₃alkyl), —N(C₁₋₃alkyl)₂ or —OC₁₋₃alkyl.

105. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, R₅₁ is selected from hydrogen.

106. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, R₅₂ is selected from —F.

107. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the prodrug is selected from any one of the following formulas: 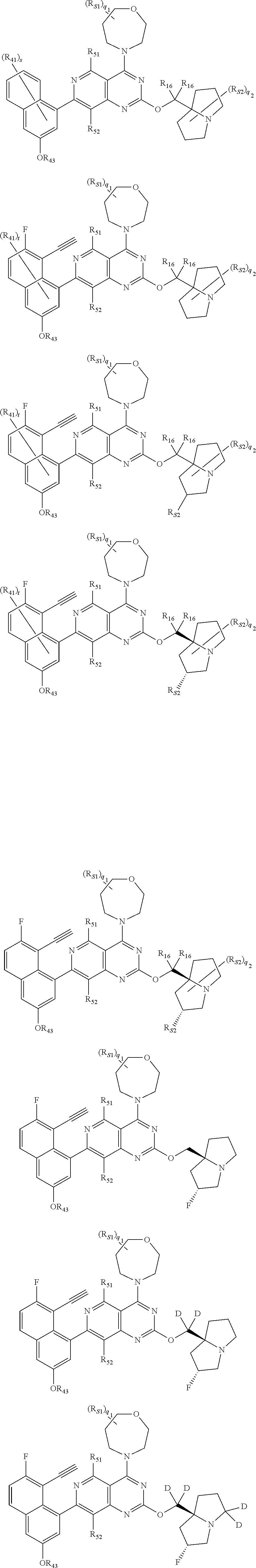
R₄₃ at each occurrence is independently selected from 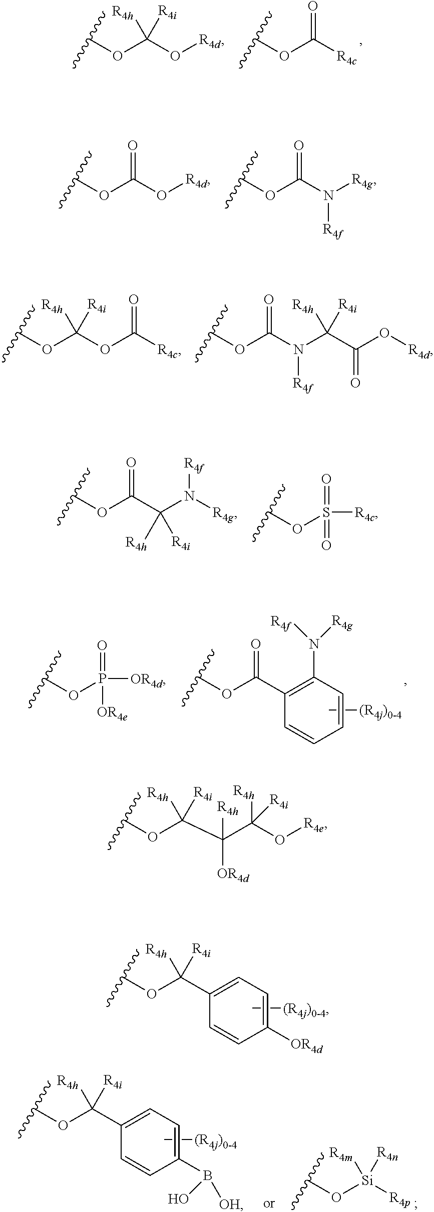
R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆ alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆ alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄ⱼ;
Optionally, R_{4f} and R₄ₕ together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄ⱼ;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆ alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆ alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂ or —OC₁₋₆alkyl;
Each of (heterocyclyl and heteroaryl) at each occurrence is independently contain 1, 2, 3 or 4 heteroatoms selected from N, O, S, S(═O) or S(═O)₂.

108. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 91, wherein, the moiety of 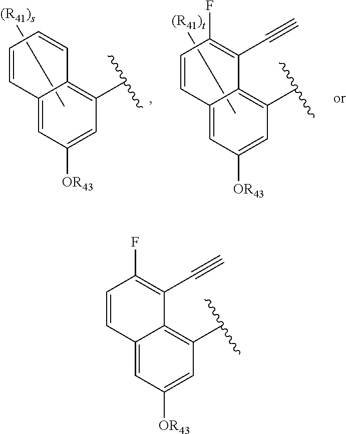
is selected from any one in the Table 12 as shown in the description.

109. The compound of formula (I) or (II), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of claim 91 selected from any compound in the Table 18 as shown in the description.

110. A pharmaceutical composition, comprising a therapeutically effective amount of the compound of formula (I) or (II), a stereoisomer thereof, a pharmaceutically acceptable salt thereof, a pharmaceutically acceptable salt of the stereoisomer, a pharmaceutical acceptable salt of the atropisomer, a prodrug thereof, a deuterated molecule thereof or a conjugated form thereof of claim 91, and a pharmaceutically acceptable excipient.
